(19) United States
(12) Patent Application Publication
(10) Pub. No.: US 2024/0352073 A1
(43) Pub. Date: Oct. 24, 2024

(54) BICYCLIC PEPTIDE INHIBITORS OF INTERLEUKIN-23 RECEPTOR

(71) Applicants: Janssen Biotech, Inc., Horsham, PA (US); Protagonist Therapeutics, Inc., Newark, CA (US)

(72) Inventors: Stephanie A. Barros, Chalfont, PA (US); Charles Hendrick, North Wales, PA (US); Santhosh Neelamkavil, Edison, NJ (US); Raymond J. Patch, Yardley, PA (US); Sandeep Somani, Princeton, NJ (US); Chengzao Sun, Warrington, PA (US); Jing Zhang, Wilmington, DE (US); Elisabetta Bianchi, Pomezia (Rome) (IT); Danila Branca, Pomezia (Rome) (IT); Roberto Costante, Pomezia (Rome) (IT); Raffaele Ingenito, Pomezia (Rome) (IT); Ashok Bhandari, Pleasanton, CA (US); Brian Frederick, Ben Lomond, CA (US); Tran Trung Tran, Newark, CA (US); Jie Zhang, Newark, CA (US)

(21) Appl. No.: 18/579,033

(22) PCT Filed: Jul. 14, 2022
(86) PCT No.: PCT/US2022/037202

§ 371 Date: Jan. 12, 2024

Related U.S. Application Data
(60) Provisional application No. 63/221,854, filed on Jul. 14, 2021.

(51) Int. Cl. C07K 7/56; A61K 38/00

(52) U.S. Cl. CPC C07K 7/56; A61K 38/00

(57) ABSTRACT

The present invention relates to novel bicyclic peptide inhibitors of the interleukin-23 receptor (IL-23R) or pharmaceutically acceptable salt thereof, corresponding pharmaceutical compositions, methods and/or uses for treatment of autoimmune inflammation and related diseases and disorders.

### CROSS-REFERENCE TO RELATED APPLICATION

This application claims the benefit under 35 U.S.C. § 119 of U.S. Provisional Application No. 63/221,854, filed Jul. 14, 2021 (pending), which is herein incorporated by reference in its entirety, including its respective sequence listing.

### PARTIES TO A JOINT RESEARCH AGREEMENT

The present disclosure was made by, or on behalf of, the below listed parties to a joint research agreement. The joint research agreement was in effect on or before the date the claimed invention was made, and the claimed invention was part of the joint research agreement and made as a result of activities undertaken within the scope of the joint research agreement. The parties to the joint research agreement are JANSSEN BIOTECH, INC. and PROTAGONIST THERAPEUTICS, INC.

### INCORPORATION OF SEQUENCE LISTING

The sequence listing in ST.26 XML format entitled 2948-15_ST26.xml, created on Jul. 13, 2022, comprising 1,941,221 bytes, prepared according to 37 CFR 1.822 to 1.824, submitted concurrently with the filing of this application, is incorporated herein by reference in its entirety.

### FIELD OF THE INVENTION

The present invention relates to novel bicyclic peptide inhibitors of the interleukin-23 receptor (IL-23R) or pharmaceutically acceptable salts, solvates and/or other forms thereof, invention relates to corresponding pharmaceutical compositions, methods and/or uses of the IL-23R inhibitors for treatment of autoimmune inflammation diseases and/or related disorders.

### BACKGROUND

The interleukin-23 (IL-23) cytokine has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs), for example, ulcerative colitis and Crohn's disease. Studies in acute and chronic mouse models of IBD revealed a primary role of interleukin-23 receptor (IL-23R) and downstream effector cytokines in disease pathogenesis. IL-23R is expressed on various adaptive and innate immune cells including Th17 cells, γδ T cells, natural killer (NK) cells, dendritic cells, macrophages, and innate lymphoid cells, which are found abundantly in the intestine. At the intestine mucosal surface, the gene expression and protein levels of IL-23R are found to be elevated in IBD patients. It is believed that IL-23 mediates this effect by promoting the development of a pathogenic CD4⁺ T cell population that produces IL-6, IL-17, and tumor necrosis factor (TNF).

Production of IL-23 is enriched in the intestine, where it is believed to play a key role in regulating the balance between tolerance and immunity through T-cell-dependent and T-cell-independent pathways of intestinal inflammation through effects on T-helper 1 (Th1) and Th17-associated cytokines, as well as restraining regulatory T-cell responses in the gut, favoring inflammation. In addition, polymorphisms in the IL-23 receptor (IL-23R) have been associated with susceptibility to inflammatory bowel diseases (IBDs), further establishing the critical role of the IL-23 pathway in intestinal homeostasis.

Psoriasis, a chronic skin disease affecting about 2%-3% of the general population has been shown to be mediated by the body's T cell inflammatory response mechanisms. IL-23 has one of several interleukins implicated as a key player in the pathogenesis of psoriasis, purportedly by maintaining chronic autoimmune inflammation via the induction of interleukin-17, regulation of T memory cells, and activation of macrophages. Expression of IL-23 and IL-23R has been shown to be increased in tissues of patients with psoriasis, and antibodies that neutralize IL-23 showed IL-23-dependent inhibition of psoriasis development in animal models of psoriasis.

IL-23 is a heterodimer composed of a unique p19 subunit and the p40 subunit shared with IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals deficient in IL-12 are susceptible to inflammatory autoimmune diseases, whereas IL-23 deficient animals are resistant, presumably due to a reduced number of CD4⁺ T cells producing IL-6, IL-17, and TNF in the CNS of IL-23-deficient animals. IL-23 binds to IL-23R, which is a heterodimeric receptor composed of IL-12R$1 and IL-23R subunits. Binding of IL-23 to IL-23R activates the Jak-Stat signaling molecules, Jak2, Tyk2, and Stat1, Stat 3, Stat 4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naïve CD4(+) T cells, IL-23 preferentially acts on memory CD4(+) T cells.

Therapeutic moieties that inhibit the IL-23 pathway have been developed for use in treating IL-23-related diseases and disorders. A number of antibodies that bind to IL-23 or IL-23R have been identified, including ustekinumab, which has been approved for the treatment of moderate to severe plaque psoriasis (PSO), active psoriatic arthritis (PSA), moderately to severely active Crohn's disease (CD) and moderately to severely active ulcerative colitis (UC). Examples of such identified antibodies, include: Tildrakizumab, an anti-IL23 antibody approved for treatment of plaque psoriasis, Guselkumab, an anti-IL23 antibody approved for treatment of psoriatic arthritis and Risankizumab, an anti-IL23 antibody approved for the treatment of plaque psoriasis in the US, and generalized pustular psoriasis, erythrodermic psoriasis and psoriatic arthritis in Japan.

Although targeted IL-23 antibody therapeutics are used clinically, there are no small-molecule therapeutics that selectively inhibit IL-23 signaling. There are some identified polypeptide inhibitors that bind to IL-23R and inhibit binding of IL-23 to IL-23R (see, e.g., US Patent Application Publication No. US2013/0029907).

Thus, there remains a significant need in the art for effective small-molecule and/or polypeptide therapeutic agents to treat and/or prevent IL-23-associated and/or IL23R-associated diseases and disorders, which include, but are not limited to psoriasis, psoriatic arthritis, inflammatory bowel diseases, ulcerative colitis, and Crohn's disease. In particular:
compounds and methods for specific targeting of IL-23R from the luminal side of the gut may provide therapeutic benefit to IBD patients suffering from local inflammation of the intestinal tissue; and/or
orally bioavailable small molecule and/or polypeptide inhibitors of IL-23 may provide both a non-steroidal treatment option for patients with mild to moderate psoriasis and treatment for moderate to severe psoriasis that does not require delivery by infusion.

Compounds and methods for specific targeting of the IL-23R from the luminal side of the gut may provide therapeutic benefit to IBD patients suffering from local inflammation of the intestinal tissue. In addition, orally bioavailable small molecule and/or polypeptide inhibitors of IL-23 may provide both a non-steroidal treatment option for patients with mild to moderate psoriasis and treatment for moderate to severe psoriasis that does not require delivery by infusion.

The present invention is directed to addressing these needs by providing bicyclic peptide inhibitors or pharmaceutically acceptable salts, solvates and/or other forms thereof, that bind IL-23R to inhibit IL-23 binding and signaling, via different suitable routes of administration, which may include but is not limited to oral administration.

### BRIEF SUMMARY

In general, the present invention relates to novel bicyclic peptide inhibitors of the interleukin-23 receptor (IL-23R) or pharmaceutically acceptable salts, solvates and/or other forms thereof, corresponding pharmaceutical compositions, methods and/or uses of the IL-23R inhibitors for treatment of autoimmune inflammation diseases and/or related disorders.

In particular, the present invention invention relates to a compound of Formulas (I′), (I) to (III)), or pharmaceutically acceptable salts, solvates and/or other forms thereof. corresponding pharmaceutical compositions, methods and/or uses for treatment of autoimmune inflammation diseases and related disorders.

The bicyclic peptide inhibitor(s) of the IL-23R of the present invention is represented by linear form structure of Formula (I′):

(I′)
R1-X3-X4-X5-X6-X7-X8-X9-X10-X11-
X12-X13-X14-X15-X16-X17-R2

The linear form structure of Formula (I′) is intended for exemplary and non-limiting purposes, which will be apparent from examples set forth and exemplified throughout the instant specification, i.e., e.g., where each such structure may be longer or shorter than the length of eighteen amino acids and/or other corresponding chemical moieties or functional group substituents as defined herein.

Specifically in Formula (I′):
X3-X17, respectively and individually, represent individual amino acid (aa) residues or other corresponding chemical moieties or functional group substituents as described below and in the instant invention;
R1 represents the N-terminal end, which may be, for example a hydrogen or a chemical moiety or functional group substituted on the amino group;
Similarly, R2 represents the carboxyl end, which may be, for example the OH of the carboxyl or a chemical moiety or functional group attached thereto or substituted for the OH group (e.g., an amino group to give a terminal amide e.g., —C(O)HN₂).
Any of the residues as shown in the linear form structure may be present or absent, e.g., X3 and/or X16-X18 may be absent;
The peptide inhibitors may have a bond between positions X4 and X9 (e.g., a pair of Pen residues or Abu and Cys residues) forming a disulfide bond or thioether bond resulting in formation of a first ring of the bicyclic ring structures, however the bond forming the first ring of the bicyclic structure may, be located between other amino acids or chemical moieties besides X4 and X9; and/or
The bond forming the second ring of the bicyclic ring structure may result in a ring that bridges the first ring structure or a separate ring structure connected by an intervening portion of the molecule.

In other aspects, a second ring of the bicyclic structure may be formed by a bond between X3 and one of X10, X13, X15, X16, or X17. In further aspects, peptides may have a second ring of the bicyclic structure provided by a bond between X5 and X10. A second ring of the bicyclic structure may also be provided by a bond between X8 and X12. Also included are bicyclic peptides having a second ring of the bicyclic structure provided by a bond between X10 and one of X13, X15, X16, R2, or R3. In aspects, a bond between X13 and either X15 or X16 forms a second ring of the bicyclic structure. In a further aspect, a second ring of the bicyclic structure provided by a bond between R1 and R2. Further details are provided below.

The present invention relates to compounds of Formulas (I′), (I) to (XX), their salts, solvates, or forms thereof, corresponding pharmaceutical compositions, and methods and/or uses for treatment of autoimmune inflammation diseases and related disorders.

In particular, the present invention relates to peptide inhibitor of the IL-23R or a pharmaceutically acceptable salt(s), solvate(s) and/or other form(s) thereof, corresponding pharmaceutical compositions, methods and/or uses for treatment of disease including autoimmune inflammation diseases and related disorders; where:
the inhibitor of the IL-23R of the present invention is identified by Formulas (I′), (I) to (XX); or
in Table 1A, Table 1B, Table 1C, Table 1D, Table 1E, Table 1F, Table 1G and Table 1H, respectively, in the present specification.

The present invention relates to compounds which are bicyclic inhibitors of an IL-23 receptor comprising an amino acid sequence of Formula XIX

(XIX)
R1-X3-X4-X5-T-X7-X8-X9-X10-X11-
X12-X13-X14-X15-X16-X17-R2

wherein:
R1 is 7Ahp, 6Ahx, 5Ava, PEG2, AEEP, AEEP(Ns), GABA, pFS, bAla, PEG2PEGE2gEC160H, C₁ to C₄ alkyl C(O)—, or C₁ to C₄ alkyl C(O)— substituted with Cl, F, or cyano, 5cpaCO, cPEG3aCO, or —H
X3 is dR, R, G, R5H, R6H, R7H, S5H, S6H, S7H, K, dK, Orn, dOrn, Dap, dDap, Dab, dDab, Dab(COCH2), dDab(COCH2), hE, dhE, hK, dhK, dK(Me)3, K(Me)3, dK(PEG2PEG2gEC180H), K(PEG2PEG2gEC180H), or absent;
X4 is Pen, Abu, or C;
X5 is N, Q, N(N(Me)2), or K(PEG2PEG2gEC180H);
X7 is W, or 7MeW,
X8 is K(Ac), dK(Ac), Q, dQ, K(NMeAc), dK(NMeAc), K(PEG2PEG2gEC180H), or dK(PEG2PEG2gEC180H);
X9 is Pen, Abu, or C;
X10 is AEF, or TMAPF;
X11 is 2Nal;
X12 is THP, Acpx, or aMeK;
X13 is E, dE, hE, dhE, aMeE, d-aMeE, D, dD, Aad, dAad, K(Ac), dK(Ac), K, dK, hSer, dhSer, Dap(pF), R5H, R6H, R7H, S5H, S6H, S7H, C, dC, K(NMe), or dK(NMe),
X14 is N;
X15 is 3Pal, H, dH, 3MeH, 3MedH, F, dF, aMeF, aMedF, THP, bAla, NMeTyr, NMedY, K, dK;
X16 is meG, NMedY, NMeK(PEG2PEG2gECI80H), NMedK(PEG2PEG2gEC180H), or absent
X17 is absent or K(PEG2PEG2gEC180H);
R2 is —NH₂, N(H)(C₁-C₄ alkyl), —HN(C₁-C₄ alkyl), —N(C₁-C₄ alkyl)₂, each alkyl optionally substituted with Cl, F, or cyano;
wherein the bicyclic peptide inhibitor of an interleukin-23 receptor is cyclized by a first disulfide or thioether bond between X4 and X9, and a second amide, aliphatic (generated from a Ring Closing Metathesis “RCM” reaction), alkyl amine, or thioether linkage between R1 and X13 or between X3 and X13.

The present invention also relates to compounds of Formula XIX, their salts, solvates, or forms thereof, corresponding pharmaceutical compositions, and methods and/or uses for treatment of autoimmune inflammation diseases and related disorders.

The present invention relates to compounds which are bicyclic inhibitors of an IL-23 receptor comprising an amino acid sequence of Formula XX

(XX)
R1-X3-X4-X5-X6-X7-X8-X9-X10-X11-
X12-X13-X14-X15-X16-R2

wherein:
R1 is selected from CF3CO, 5cpaCO, cPEG3aCO, —H, C1 to C4 alkyl C(O)—, or C1 to C4 alkyl C(O)— substituted with cyano, Cl, or F;
X3 is R, dR, K, dK, K(Me)3, dK(Me)3, hK(Me)3, dhK(Me)3, or absent
X4 is Pen, Abu, or C;
X5 is selected from E, D, K, K(Ac), Dap, or K(NMe), K(NNs);
X6 is selected from T, L;
X7 is selected from W, 7MeW, 7PhW;
X8 is selected from K(Ac), dK(Ac), hK(Me)3, dhK(Me)3, K(Me)3, dK(Me)3, K(NMeAc), dK(NMeAc), Q(N(Me)2), KPeg12, dKPeg12, KAcMor, A, Q, dKacMor, dQ(N(Me)2), K(mPEG12), dA, dQ, or dK(mPEG12);
X9 is Pen, Abu, or C;
X10 is selected from AEF, AEF(NMe), F4CONH2, or F40Me;
X11 is 2Nal;
X12 is selected from THP, aMeLeu, or A;
X13 is selected from E, dE, K(Ac), dK(Ac), K(Me)3, dK(Me)3, K(NMeAc), dK(NMeAc), Q(N(Me)2), dQ(N(Me)2), A, dA, L, or dL;
X14 is selected from L, N or S;
X15 is selected from 3Pal, L, dL, or Aib
X16 is selected from meG; and
R2 is —NH₂, N(H)(C₁-C₄ alkyl), —HN(C₁-C₄ alkyl), —N(C₁-C₄ alkyl)₂, each alkyl optionally substituted with Cl, F, or cyano; and
   wherein the bicyclic peptide inhibitor of an interleukin-23 receptor is cyclized by a first disulfide or thioether bond between X4 and X9, and a second amide or alkyl amine linkage between X5 and X10.

The present invention also relates to compounds of Formula XX, their salts, solvates, or forms thereof, corresponding pharmaceutical compositions, and methods and/or uses for treatment of autoimmune inflammation diseases and related disorders.

The present invention relates to compounds which are bicyclic inhibitors of an IL-23 receptor comprising an amino acid sequence of Formula I

(I)
R1-X4-X5-T-X7-X8-X9-AEF-X11-X12-
X13-N-X15-meG-R2

wherein:
   R1 is 7Ahp, 6Ahx, 5Ava, PEG2, AEEP, or AEEP(Ns);
   X4 is Pen, Abu, aMeC, hC, or C;
   X5 is N or K(PEG2PEG2gEC180H);
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is K(Ac), Q, K(NMeAc), K(PEG2PEG2gEC180H), dK(Ac), dQ, dK(NMeAc), or dK(PEG2PEG2gEC180H);
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is THP or aMeK;
   X13 is E, dE, hE, dhR, D, dD, hSer, or dhSer;
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
   R2 is —NH₂, N(H)C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond or thioether bond (between pFS and Dap(pF)) between R1 and X13.

The present invention also relates to compounds of Formula I, their salts, solvates, or forms thereof, corresponding pharmaceutical compositions, and methods and/or uses for treatment of autoimmune inflammation diseases and related disorders.

The present invention relates to compounds which are bicyclic inhibitors of an IL-23 receptor comprising an amino acid sequence of Formulas II-XVIII.

The present invention also relates to compounds of Formula II-XVIII, their salts, solvates, or forms thereof, corresponding pharmaceutical compositions, and methods and/or uses for treatment of autoimmune inflammation diseases and related disorders.

The present invention relates to compounds which are bicyclic inhibitors of an IL-23 receptor comprising an amino acid sequence of Formula III.

In addition to the foregoing, the present invention relates to methods or processes of making compound of Formulas (I) to (XX) or Tables 1A to 1H).

The present invention also relates to pharmaceutical composition(s), which comprises a herein-described bicyclic peptide inhibitor compound of the IL-23R or a pharmaceutically acceptable salt, solvate, or form thereof as described herein, and a pharmaceutically acceptable carrier, excipient, or diluent. The pharmaceutical compositions may comprise or may exclude an absorption enhancer depending on the intended route of delivery or use thereof for treatment of specific indications. The absorption enhancer may be permeation enhancer or intestinal permeation enhancer. In an aspect the absorption enhancer improves oral bioavailability.

The present invention relates to method(s) for treating and/or uses(s) for inflammatory disease(s) in a subject, which comprises administering a therapeutically effective amount of one or more herein-described bicyclic peptide inhibitor compounds of the IL-23R or pharmaceutically acceptable salts, or solvates thereof, or a corresponding pharmaceutical composition as described herein, respectively to a subject in need thereof. Such inflammatory diseases and related disorders may include, but are not limited to, inflammatory bowel disease (IBD), Crohn's disease (CD), ulcerative colitis (UC), psoriasis (PsO), or psoriatic arthritis (PsA) and the like.

The present invention provides for the use of one or more herein-described compounds (e.g., compounds of Formulas (I) to (XX) or Tables 1A to 1H)) for the preparation of pharmaceutical compositions for use in the treatment of inflammatory diseases and related disorders including, but not limited to, inflammatory bowel disease (IBD), Crohn's disease (CD), ulcerative colitis (UC), psoriasis (PsO), and psoriatic arthritis (PsA).

The present invention provides for the use of one or more herein-described compounds of Formulas (I) to (XX) in the treatment of inflammatory diseases and related disorders including, but not limited to, inflammatory bowel disease (IBD), Crohn's disease (CD), ulcerative colitis (UC), psoriasis (PsO), and psoriatic arthritis (PsA).

The present invention provides for kits comprising one or more herein-described compounds of Formulas (I) to (XX) and instructions for use in treating a disease in a patient. The disease may be an inflammatory diseases or related disorder including, but not limited to, inflammatory bowel disease (IBD), Crohn's disease (CD), ulcerative colitis (UC), psoriasis (PsO), and psoriatic arthritis (PsA).

### DETAILED DESCRIPTION

### I. General

The present invention relates to novel bicyclic peptide inhibitors of the interleukin-23 receptor (IL-23R) or pharmaceutically acceptable salts, solvates and/or other forms thereof, corresponding pharmaceutical compositions, methods and/or uses of the IL-23R inhibitors for treatment of autoimmune inflammation diseases and/or related disorders.

The present invention to relates to bicyclic cyclic peptide inhibitors of an IL-23R. The bicyclic peptide inhibitors of the present invention may exhibit enhanced properties, such as longer in vivo half-life, compared to the corresponding monocyclic peptide inhibitor of an IL-23R.

### II. Definitions

Unless otherwise defined herein, scientific and technical terms used in this application shall have the meanings that are commonly understood by those of ordinary skill in the art.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Patient” or “subject”, which are used interchangeably, refer to a living organism, which includes, but is not limited to a human subject suffering from or prone to a disease or condition that can be treated by administration of a pharmaceutical composition as provided herein. Further non-limiting examples may include, but is not limited to humans, other mammals, bovines, rats, mice, dogs, monkeys, goat, sheep, cows, deer, horse, and other mammalian animals and the like. In some aspects, the patient is human.

Unless indicated otherwise, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. In sequences of amino acids that represent IL-23 inhibitors the individual amino acids are separated by a hyphen “-” or brackets e.g, lysine is shown as [K].

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). Unless otherwise indicated, three-letter and single-letter abbreviations of amino acids refer to the L-isomeric form of the amino acid in question. The term “L-amino acid,” as used herein, refers to the “L” isomeric form of a peptide, and conversely the term “D-amino acid” refers to the “D” isomeric form of a peptide (e.g., (D)Asp or D-Asp; (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. For example, L-arginine can be represented as “Arg” or “R,” while D-arginine can be represented as “arg” or “r.” Similarly, L-lysine can be represented as “Lys” or “K,” while D-lysine can be represented as “lys” or “k.” Alternatively, a lower case “d” in front of an amino acid can be used to indicate that it is of the D isomeric form, for example D-lysine can be represented by dK.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and Abu (2-amino butyric acid).

Amino acids of the D-isomeric form may be located at any of the positions in the IL-23R inhibitors set forth herein (any of X1-X18 appearing in the molecule). In an aspects, amino acids of the D-isomeric form may be located only at any one or more of X3, X5, X6, X8, X13, X16, and optionally one additional position. In other aspects, amino acids of the D-isomeric form may be located only at any one or more of X3, X8, X13, X16, and optionally one additional position. In other aspects, amino acids of the D-isomeric form may be located only at any one or more of X8, X13 (e.g., X8 is dK(Ac) and x13 is dE), and optionally one additional position. In other aspects, amino acids of the D-isomeric form may be located only at X3, and optionally one additional position. In other aspects, amino acids of the D-isomeric form may be located only at X3, and optionally two or three additional positions. In other aspects, amino acids of the D-isomeric form may be located at only one or two of positions X1 to X18 appearing in the IL-23R inhibitors set forth herein. In other aspects, amino acids of the D-isomeric form may be located at only three or four of positions X1 to X18 appearing in the IL-23R inhibitors set forth herein. For example, an IL-23R inhibitors set forth herein having only positions X3 to X15 present may have amino acids of the D-form present in 3 or four of those positions. In other aspects, amino acids of the D-isomeric form may be located at only five or six of positions X1 to X18 appearing in the IL-23R inhibitors set forth herein.

As conventionally understood in the art or to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the invention, a C-terminal “—OH” moiety may be substituted for a C-terminal “—NH₂” moiety, and vice-versa.

One of skill in the art will appreciate that certain amino acids and other chemical moieties are modified when bound to another molecule. For example, an amino acid side chain may be modified when it forms an intramolecular bridge with another amino acid side chain, e.g., one or more hydrogen may be removed or replaced by the bond.

A “compound of the invention”, an “inhibitor of the present invention”, an “IL-23R inhibitor of the present invention”, a “compound described herein”, and a “herein-described compound” include the novel compounds disclosed herein, for example the compounds of any of the Examples, including compounds of Formulas (I) to (XX) such as those found in Table 1A, Table 1B, Table 1C, Table 1D, Table 1E, Table 1F, Table 1G or Table 1H.

“Pharmaceutically effective amount” refers to an amount of a compound of the invention in a composition or combination thereof that provides the desired therapeutic or pharmaceutical result.

By “pharmaceutically acceptable” it is meant the carrier(s), diluent(s), salts, or excipient(s) must be compatible with the other components or ingredients of the compositions of the present invention, i.e., that which is useful, safe, non-toxic acceptable for pharmaceutical use. In accordance with the present invention pharmaceutically acceptable means approved or approvable as is listed in the U.S. Pharmacopoeia or other generally recognized pharmacopoeia for use in animals, and more particularly, in humans.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Absorption enhancer” refers to a component that improves or facilitates the mucosal absorption of a drug in the gastrointestinal tract, such as a permeation enhancer or intestinal permeation enhancer. As conventionally understood in the art, permeation enhancers (PEs) are agents aimed to improve oral delivery of therapeutic drugs with poor bioavailability. PEs are capable of increasing the paracellular and/or transcellular passage of drugs.

Pharmaceutical excipients that can increase permeation have been termed “absorption modifying excipients” (AMEs). AMEs may be used in oral compositions, for example, as wetting agents (sodium dodecyl sulfate), antioxidants (e.g. EDTA), and emulsifiers (e.g. macrogol glycerides), and may be specifically included in compositions as PEs to improve bioavailability. PEs can be categorized as to how they alter barrier integrity via paracellular or transcellular routes.

“Intestinal permeation enhancer (IPE)” refers to a component that improves the bioavailability of a component. Suitable representative IPEs for use in the present invention, include, but are not limited to, various surfactants, fatty acids, medium chain glycerides, steroidal detergents, acyl carnitine and alkanoylcholines, N-acetylated alpha-amino acids and N-acetylated non-alpha-amino acids, and chitosans, other mucoadhesive polymers and the like. For example, a suitable IPE for use in the present invention may be sodium caprate.

“Composition” or “Pharmaceutical Composition” as used herein is intended to encompass an invention or product comprising the specified active product ingredient (API), which may include pharmaceutically acceptable excipients, carriers or diluents as described herein, such as in specified amounts defined throughout the invention. Compositions or Pharmaceutical Compositions result from combination of specific components, such as specified ingredients in the specified amounts as described herein.

Compositions or pharmaceutical compositions of the present invention may be in different pharmaceutically acceptable forms, which may include, but are not limited to a liquid composition, a tablet or matrix composition, a capsule composition, etc. and the like. When the composition is a tablet composition, the tablet may include, but is not limited to different layers two or more different phases, including an internal phase and an external phase that can comprise a core. The tablet composition can also include, but is not limited to, one or more coatings.

“Solvate” as used herein, means a physical association of the compound of the present invention with one or more solvent molecules. This physical association involves varying degrees bonding, including hydrogen bonding. In certain instances, the solvate will be capable of isolation. The term “solvate” is intended to encompass both solution-phase and isolatable solvates. Non-limiting examples of suitable solvates include hydrates.

Provided are also pharmaceutically acceptable salts and tautomeric forms of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The IL-23R inhibitors of the present invention, or their pharmaceutically acceptable salts or solvates may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms of the IL-23R inhibitors of the present invention. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the aspect encompasses, but is not limited to, the specific diastereomerically or enantiomerically enriched form. Where chirality is not specified but is present, it is understood that the aspect is directed to either the specific diastereomerically or enantiomerically enriched form; or a racemic or scalemic mixture of such compound(s). As used herein, “scalemic mixture” is a mixture of stereoisomers enantiomers at a ratio other than 1:1.

“Racemates” refers to a mixture of enantiomers. The mixture can include equal or unequal amounts of each enantiomer.

“Stereoisomer” and “stereoisomers” refer to compounds that differ in the chirality of one or more stereo centers. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

Unless defined otherwise, all technical and scientific terms used herein have the same meaning as commonly or conventionally understood by one of ordinary skill in the art. In the chemical arts a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

“Treatment” or “treat” or “treating” as used herein refers to an approach for obtaining beneficial or desired results. For purposes of the present invention, beneficial or desired results include, but are not limited to, alleviation of a symptom and/or diminishment of the extent of a symptom and/or preventing a worsening of a symptom associated with a disease or condition. In one aspect, “treatment” or “treating” includes one or more of the following: (a) inhibiting the disease or condition (e.g., decreasing one or more symptoms resulting from the disease or condition, and/or diminishing the extent of the disease or condition); (b) slowing or arresting the development of one or more symptoms associated with the disease or condition (e.g., stabilizing the disease or condition, delaying the worsening or progression of the disease or condition); and (c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of a compound that, when administered to a subject for treating a disease is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some aspects, a unit dose of a compound of the invention is administered first, followed within seconds or minutes by administration of a unit dose of one or more additional therapeutic agents. Alternatively, in other aspects, a unit dose of one or more additional therapeutic agents is administered first, followed by administration of a unit dose of a compound of the invention within seconds or minutes. In some aspects, a unit dose of a compound of the invention is administered first, followed, after a period of hours (e.g., 1-12 hours), by administration of a unit dose of one or more additional therapeutic agents. In other aspects, a unit dose of one or more additional therapeutic agents is administered first, followed, after a period of hours (e.g., 1-12 hours), by administration of a unit dose of a compound of the invention. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

Abbreviation, “(V/V)” refers to the phrase “volume for volume”, i.e., the proportion of a particular substance within a mixture, as measured by volume or a volume amount of a component of the composition disclosed herein relative to the total volume amount of the composition. Accordingly, the quantity is unit less and represents a volume percentage amount of a component relative to the total volume of the composition. For example, a 2% (V/V) solvent mixture can indicate 2 mL of one solvent is present in 100 mL of the solvent mixture.

Abbreviation, “(w/w)” refers to the phrase “weight for weight”, i.e., the proportion of a particular substance within a mixture, as measured by weight or mass or a weight amount of a component of the composition disclosed herein relative to the total weight amount of the composition. Accordingly, the quantity is unit less and represents a weight percentage amount of a component relative to the total weight of the composition. For example, a 2% (w/w) solution can indicate 2 grams of solute is dissolved in 100 grams of solution.

Systemic routes of administration as conventionally understood in the medicinal or pharmaceutical arts, refer to or are defined as a route of administration of drug, a pharmaceutical composition or formulation, or other substance into the circulatory system so that various body tissues and organs are exposed to the drug, formulation or other substance. As conventionally understood in the art, administration can take place orally (where drug or oral preparations are taken by mouth, and absorbed via the gastrointestinal tract), via enteral administration (absorption of the drug also occurs through the gastrointestinal tract) or parenteral administration (generally injection, infusion, or implantation, etc.

“Systemically active” peptide drug therapy as it relates to the present invention generally refers to treatment by means of a pharmaceutical composition comprising a peptide active ingredient, wherein said peptide resists immediate metabolism and/or excretion resulting in its exposure in various body tissues and organs, such as the cardiovascular, respiratory, gastrointestinal, nervous or immune systems.

Systemic drug activity in the present invention also refers to treatment using substances that travel through the bloodstream, reaching and affecting cells in various body tissues and organs. Systemic active drugs are transported to their site of action and work throughout the body to attack the physiological processes that cause inflammatory diseases.

“Bioavailability” refers to the extent and rate at which the active moiety (drug or metabolite) enters systemic circulation, thereby accessing the site of action. Bioavailability of a drug is impacted by the properties of the dosage form, which depend partly on its design and manufacture.

“Digestive tract tissue” as used herein refers to all the tissues that comprise the organs of the alimentary canal. For example, only, and without limitation, “digestive tract tissue” includes tissues of the mouth, esophagus, stomach, small intestine, large intestine, duodenum, and anus.

### III. Compounds

The present invention relates to novel bicyclic peptide inhibitors of the interleukin-23 receptor (IL-23R) or pharmaceutically acceptable salt thereof.

In particular, the present invention relates to a bicyclic peptide inhibitors of the interleukin-23 receptor (IL-23R) or a pharmaceutically acceptable salt thereof, including those for which a structure is as identified in Table 1A, Table 1B, Table 1C, Table 1D, Table 1E, Table 1F, Table 1G, and Table 1H.

In one aspect, a bicyclic peptide inhibitor compound of the interleukin-23 receptor (IL-23R) compound, or a pharmaceutically acceptable salt thereof, has a structure of a compound in Table 1A.

In another aspect, a bicyclic peptide inhibitor compound of the interleukin-23 receptor (IL-23R) compound, or a pharmaceutically acceptable salt thereof, has a structure of a compound in Table 1B.

In another aspect, a bicyclic peptide inhibitor compound of the interleukin-23 receptor (IL-23R) compound, or a pharmaceutically acceptable salt thereof, has a structure of a compound in Table 1C.

In another aspect, a bicyclic peptide inhibitor compound of the interleukin-23 receptor (IL-23R) compound, or a pharmaceutically acceptable salt thereof, has a structure of a compound in Table 1D.

In another aspect, a bicyclic peptide inhibitor compound of the interleukin-23 receptor (IL-23R) compound, or a pharmaceutically acceptable salt thereof, has a structure of a compound in Table 1E.

In another aspect, a bicyclic peptide inhibitor compound of the interleukin-23 receptor (IL-23R) compound, or a pharmaceutically acceptable salt thereof, has a structure of a compound in Table 1F.

In another aspect, a bicyclic peptide inhibitor compound of the interleukin-23 receptor (IL-23R) compound, or a pharmaceutically acceptable salt thereof, has a structure of a compound in Table 1G.

In another aspect, a bicyclic peptide inhibitor compound of the interleukin-23 receptor (IL-23R) compound, or a pharmaceutically acceptable salt thereof, has a structure of a compound in Table 1H.

### Table 1A. Compounds

TABLE 1A

| SEQ | | |
|---|---|---|
| ID | Cmpd. | Sequence/Structure |
| 1 | 201 | 7Ahp(2)--Pen(3)-N-T-W-K(Ac)-Pen(3)-AEF-2Nal-THP-E(2)-N-3Pya-Sar—CONH2 |
| | | 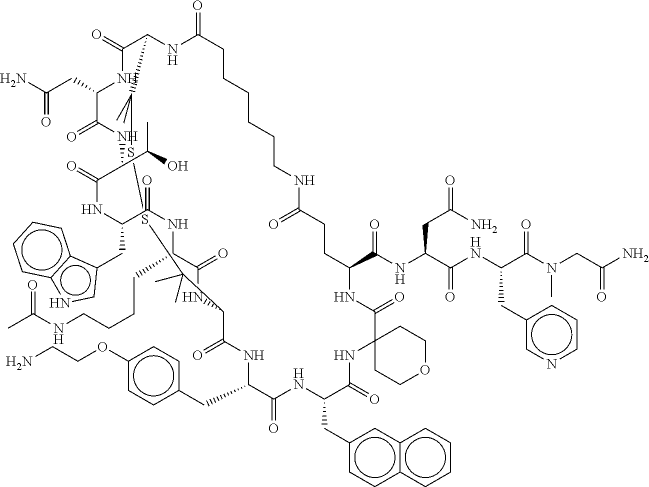 |
| 2 | 202 | 7Ahp(2)--Pen(3)-N-T-7MeW-K(Ac)-Pen(3)-AEF-2Nal-THP-E(2)-N-3Pya-Sar—CONH2 |
| | | 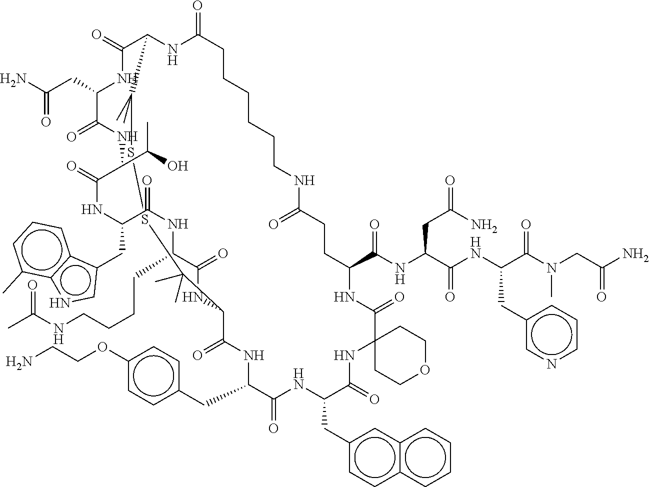 |
| 3 | 203 | 6Ahx(2)--Abu(1)-N-T-W-Q-C(1)-AEF-2Nal-THP-E(2)-N-3Pya-Sar—CONH2 |
| | | 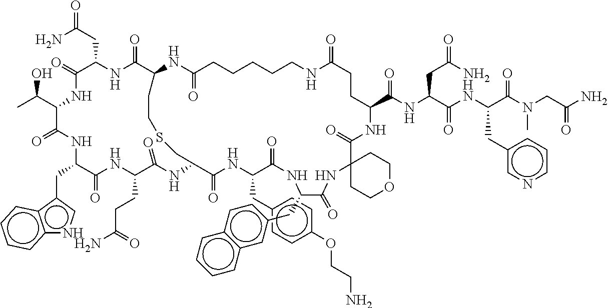 |
| 4 | 204 | MeCO-R6H(4)-Pen(3)-N-T-7MeW-K(Ac)-Pen(3)-AEF-2Nal-aMeK-S5H(4)-N-3Pya-Sar—CONH2 |
| | | 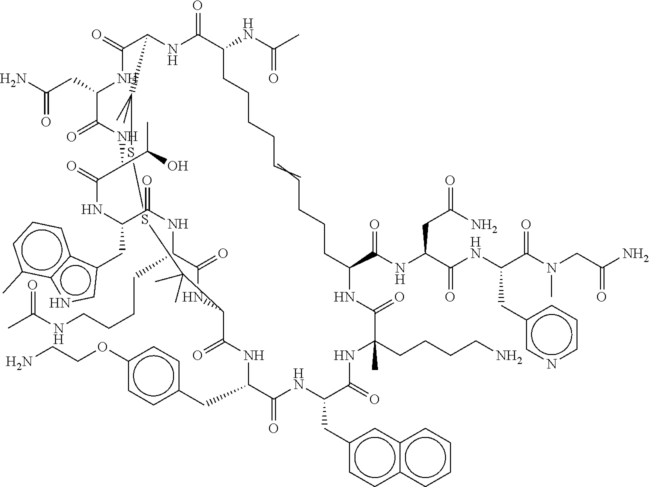 |
| 5 | 205 | 6Ahx(2)--Abu(1)-N-T-W-Q-C(1)-AEF-2Nal-aMeK-E(2)-N-3Pya-Sar—CONH2 |
| | | 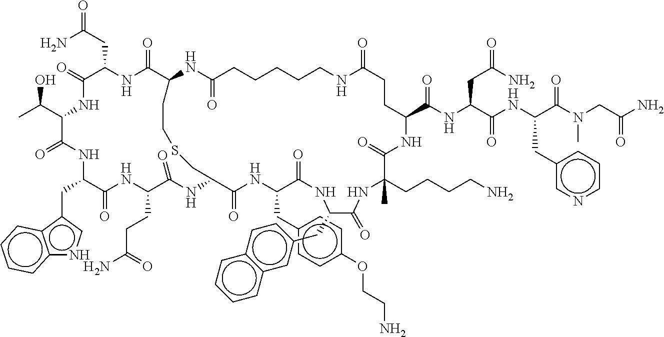 |
| 6 | 206 | GABA(2)-r-Pen(3)-N-T-7MeW-K(Ac)-Pen(3)-AEF-2Nal-THP-E(2)-N-3Pya-Sar—CONH2 |
| | | 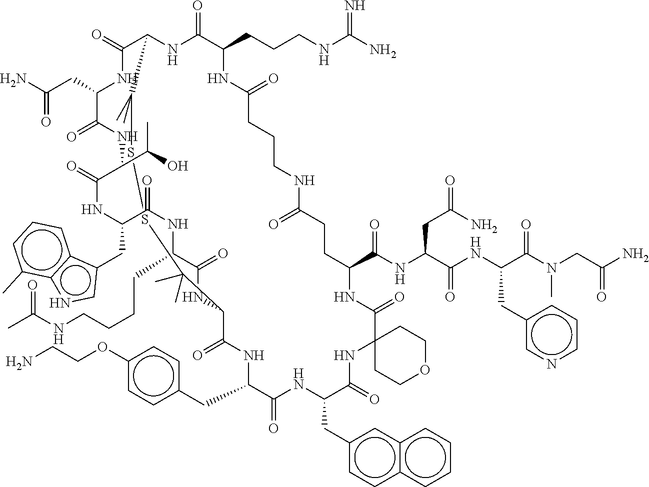 |
| 7 | 207 | MeCO-R5H(4)-Pen(3)-N-T-7MeW-K(Ac)-Pen(3)-AEF-2Nal-aMeK-S5H(4)-N-3Pya-Sar—CONH2 |
| | | 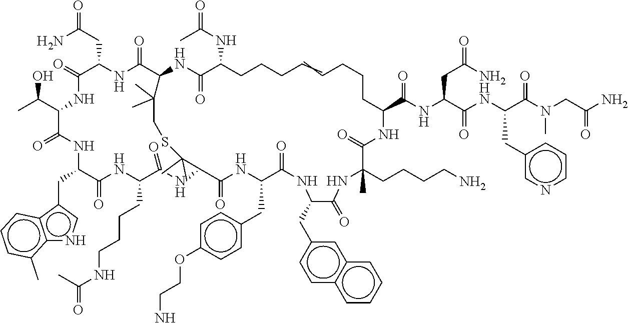 |
| 8 | 208 | MeCO-R5H(4)-Pen(3)-N-T-7MeW-K(Ac)-Pen(3)-AEF-2Nal-aMeK-S5H(4)-N-3Pya-Sar—CONH2 |
| | | 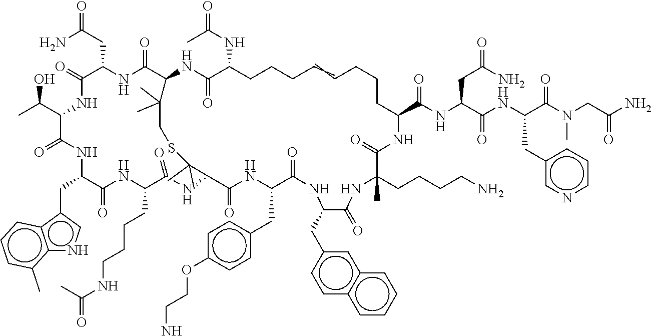 |
| 9 | 209 | 7Ahp(2)--Pen(3)-N-T-7MeW-K(Ac)-Pen(3)-AEF-2Nal-THP-E(2)-N-3Pya—CONH2 |
| | | 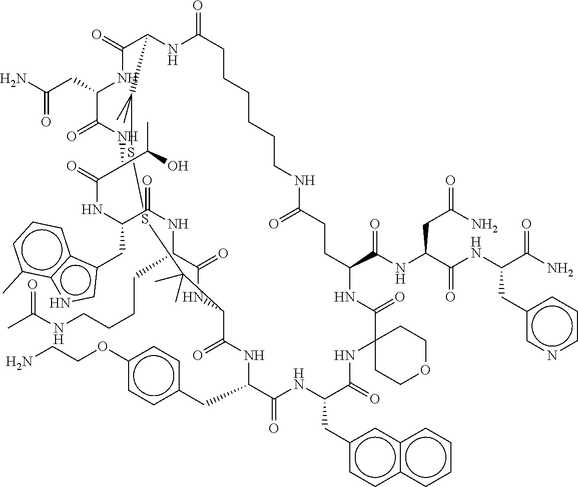 |
| 10 | 210 | MeCO-R7H(4)-Pen(3)-N-T-7MeW-K(Ac)-Pen(3)-AEF-2Nal-THP-S5H(4)-N-3Pya-Sar—CONH2 |
| | | 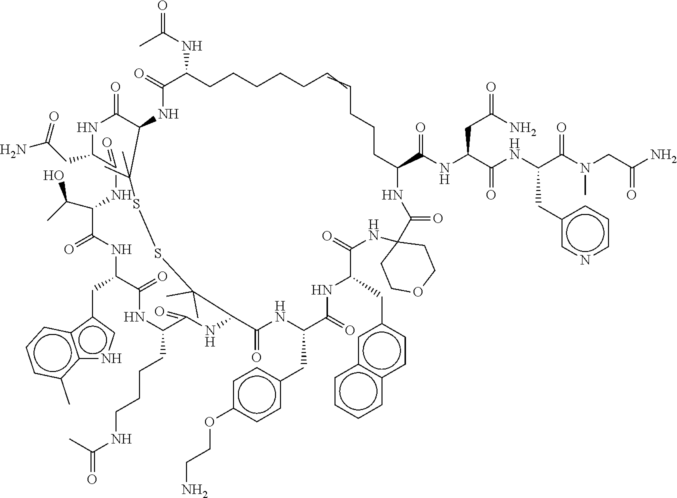 |
| 11 | 211 | MeCO-R7H(4)-Pen(3)-N-T-7MeW-K(Ac)-Pen(3)-AEF-2Nal-THP-S5H(4)-N-3Pya-Sar—CONH2 |
| | | 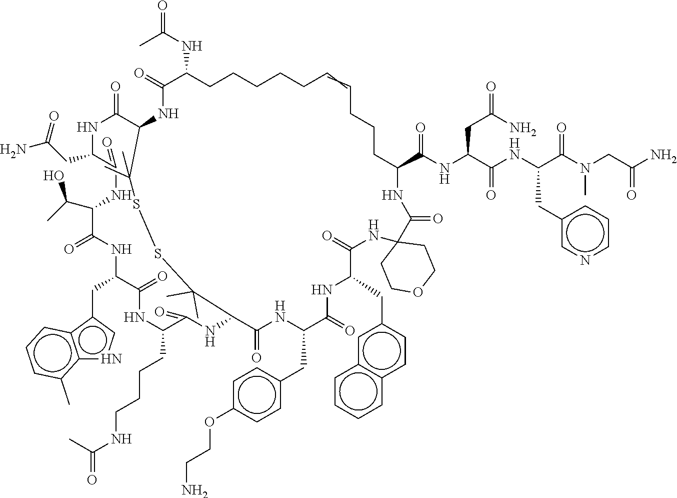 |
| 12 | 212 | 5Ava(2)--Abu(1)-N-T-W-Q-C(1)-AEF-2Nal-THP-E(2)-N-3Pya-Sar—CONH2 |
| | | 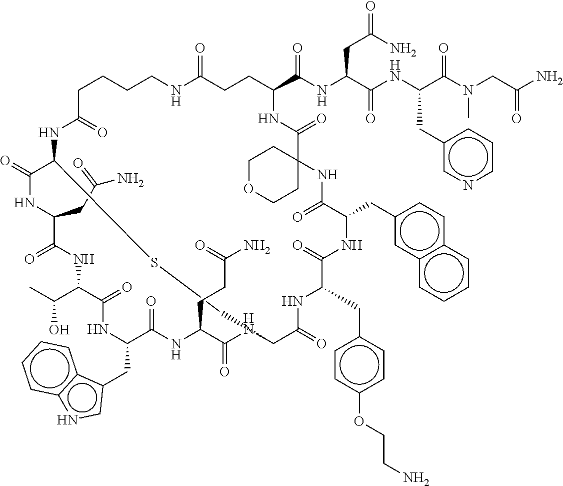 |
| 13 | 213 | 7AHP(2)-Pen(3)-N-T-7MeW-K(Ac)-Pen(3)-AEF-2Nal-THP-E(2)-N-3MeH-Sar—CONH2 |
| | | 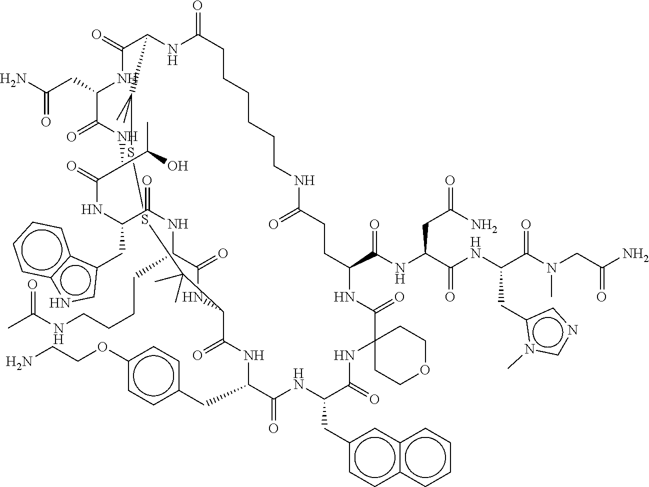 |
| 14 | 214 | -6Ahx(2)-Abu(1)-N-T-7MeW-K(Ac)-C(1)-AEF-2Nal-THP-E(2)-N-3MeH-Sar—CONH2 |
| | | 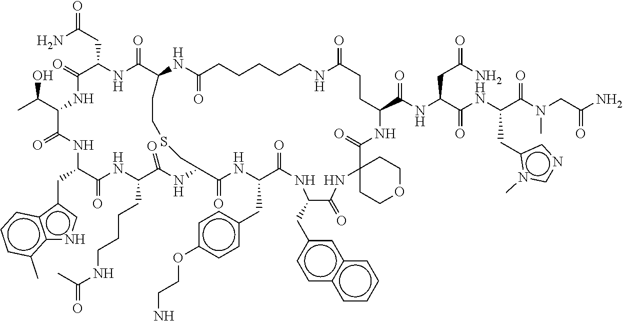 |
| 15 | 215 | MeCO-lys(2)-Pen(3)-Q-T-W-Q-Pen(3)-AEF-2Nal-THP-E(2)-N-3Pya-Sar—CONH2 |
| | | 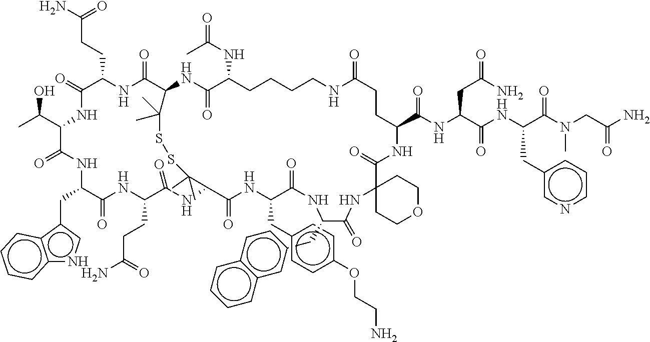 |
| 16 | 216 | MeCO-R5H(4)-Pen(3)-Q-T-W-Q-Pen(3)-AEF-2Nal-THP-S5H(4)-N-3Pya-Sar—CONH2 |
| | | 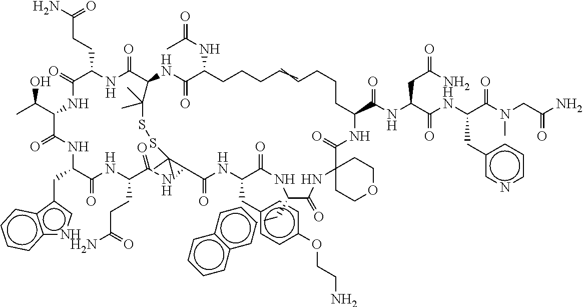 |
| 17 | 217 | pFS(6)-Gly-Pen(3)-N-T-7MeW-K(Ac)-Pen(3)-AEF-2Nal-THP-Dap(pF(6))-N-3Pya-Sar—CONH2 |
| | | 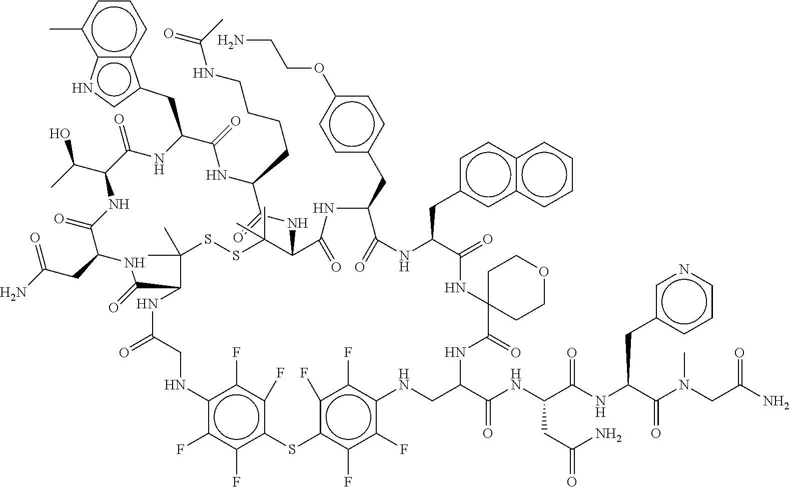 |

Wherein, for example, Pen-Pen form a disulfide bond, or Abu-C form a thioether bond.

TABLE 1B
Compounds

| SEQ | | | |
|---|---|---|---|
| ID. | Cmpd. | Sequence/Structure | Note |
| 18 | 1 | Ac-[(D)Lys]-[Pen]-Q-T-W-Q-[Pen]-[Phe(4-(2- | #N/A |
| | | aminoethoxy))]-[2Nal]-[THP]-E-N-[bAla]-NH2 | |
| | | 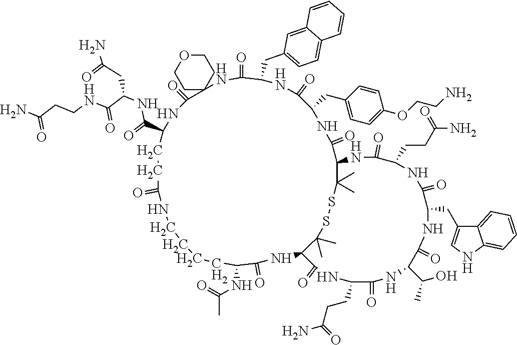 [SMILES: CC(=O)N[C@@H]1CCCCNC(=O)CC[C@@H](C(=O)N[C@@H](CC(N)=O)C(=O)NCCC(N)=O)NC(=O)C2(CCOCC2)NC(=O)[C@H](Cc2ccc3ccccc3c2)NC(=O)[C@H](Cc2ccc(OCCN)cc2)NC(=O)[C@H]2NC(=O)[C@H](CCC(N)=O)NC(=O)[C@H](Cc3c[nH]c4ccccc34)NC(=O)[C@H]([C@@H](C)O)NC(=O)[C@H](CCC(N)=O)NC(=O)[C@@H](NC1=O)C(C)(C)SSC2(C)C] | |
| 19 | 2 | Ac-[Abu]-Q-T-W-Q-C-[Phe(4-OCH2COOH)]-[2Nal]- | Glu C(O) and NH |
| | | [THP]-K-N-[bAla]-NH2 | of AEF form a |
| | | | lactam |
| | | 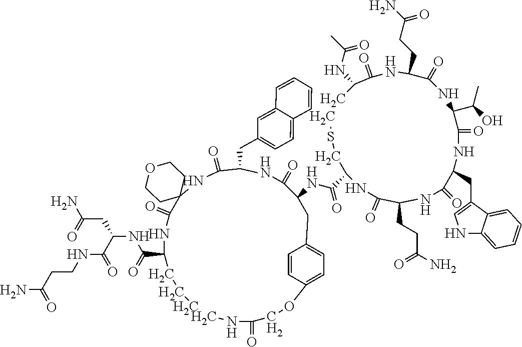 [SMILES: CC(=O)N[C@H]1CCSC[C@@H](C(=O)N[C@H]2Cc3ccc(cc3)OCC(=O)NCCCC[C@@H](C(=O)N[C@@H](CC(N)=O)C(=O)NCCC(N)=O)NC(=O)C3(CCOCC3)NC(=O)[C@H](Cc3ccc4ccccc4c3)NC2=O)NC(=O)[C@H](CCC(N)=O)NC(=O)[C@H](Cc2c[nH]c3ccccc23)NC(=O)[C@H]([C@@H](C)O)NC(=O)[C@H](CCC(N)=O)NC1=O] | |
| 20 | 3 | Ac-[(D)Arg]-[Pen]-E-T-[W(7-Me)]-[Lys(Ac)]-[Pen]- | —NH of Phe[4-(2- |
| | | [Phe(4-(2-aminoethoxy))]-[2Nal]-[THP]-[Lys(Ac)]-N- | aminoethoxy)] and |
| | | [3Pal]-[Sarc]-NH2 | C(O) of Glu form a |
| | | | lactam |
| | | 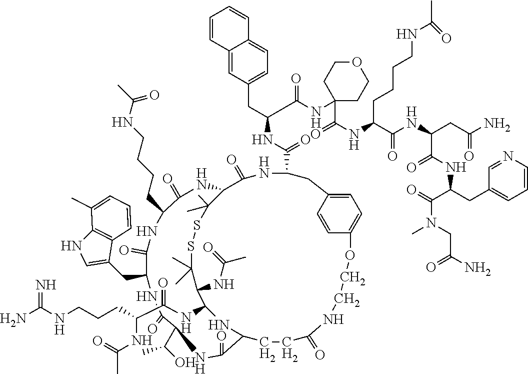 [SMILES: CC(=O)NCCCC[C@@H]1NC(=O)[C@H](Cc2c[nH]c3c(C)cccc23)NC(=O)[C@H]([C@@H](C)O)NC(=O)C2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)[C@H](NC(=O)[C@@H](CCCNC(=N)N)NC(C)=O)N2] | |
| 21 | 4 | Ac-[Pen]-E-T-W-[Lys(Ac)]-[Pen]-[Phe(4-(2- | NH of Phe[4-(2- |
| | | aminoethoxy))]-[2Nal]-[THP]-[Lys(Ac)]-S-[3Pal]-[Sarc]- | aminoethoxy)] and |
| | | NH2 | C(O) of Glu form a |
| | | | lactam Isomer 1 |
| | | 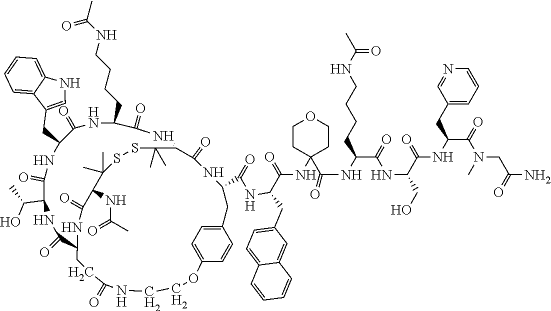 [SMILES: CC(=O)NCCCC[C@@H]1NC(=O)[C@H](Cc2c[nH]c3ccccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CO)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | |
| 22 | 5 | Ac-[Pen]-E-T-W-[Lys(Ac)]-[Pen]-[Phe(4-(2- | NH of Phe [4-(2- |
| | | aminoethoxy))]-[2Nal]-[THP]-[Lys(Ac)]-A-[3Pal]-[Sarc]-NH2 | aminoethoxy)] and |
| | | | C(O) of Glu form a |
| | | 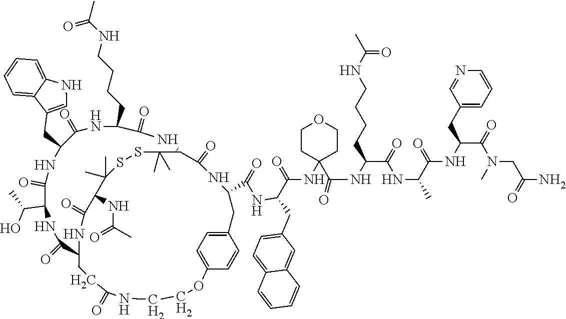 [SMILES: CC(=O)NCCCC[C@@H]1NC(=O)[C@H](Cc2c[nH]c3ccccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](C)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | lactam Isomer 1 |
| 23 | 6 | Ac-[Pen]-E-T-W-[Lys(Ac)]-[Pen]-[Phe(4-(2- | —NH of Phe [4-(2- |
| | | aminoethoxy))]-[2Nal]-[THP]-A-S-[3Pal]-[Sarc]-NH2 | aminoethoxy)] and |
| | | | C(O) of Glu form a |
| | | 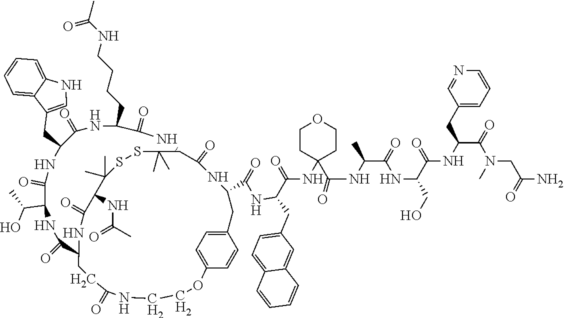 [SMILES: CC(=O)NCCCC[C@@H]1NC(=O)[C@H](Cc2c[nH]c3ccccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](C)C(=O)N[C@@H](CO)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | lactam |
| 24 | 7 | Ac-[Pen]-E-T-W-[Lys(Ac)]-[Pen]-[Phe(4-(2- | NH of Phe[4-(2- |
| | | aminoethoxy))]-[2Nal]-A-[Lys(Ac)]-S-[3Pal]-[Sarc]-NH2 | aminoethoxy)] and |
| | | | C(O) of Glu form a |
| | | 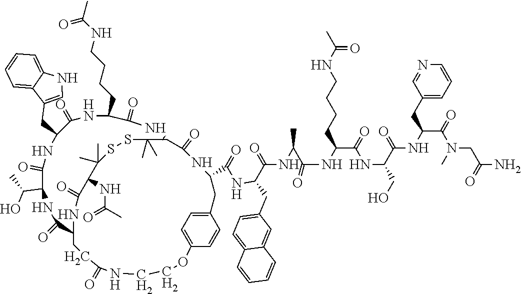 [SMILES: CC(=O)NCCCC[C@H](NC(=O)[C@H](C)NC(=O)[C@H](Cc1ccc2ccccc2c1)NC(=O)[C@@H]1Cc2ccc(cc2)OCCNC(=O)CC[C@H]2NC(=O)[C@@H](NC(C)=O)C(C)(C)SSC(C)(C)[C@H](NC(=O)[C@H](CCCCNC(C)=O)NC(=O)[C@H](Cc3c[nH]c4ccccc34)NC(=O)[C@H]([C@@H](C)O)NC2=O)C(=O)N1)C(=O)N[C@@H](CO)C(=O)N[C@@H](Cc1cccnc1)C(=O)N(C)CC(N)=O] | lactam Isomer 1 |
| 25 | 8 | Ac-[Pen]-E-T-W-[Lys(Ac)]-[Pen]-[Phe(4-(2- | NH of Phe[4-(2- |
| | | aminoethoxy))]-A-[THP]-[Lys(Ac)]-S-[3Pal]-[Sarc]-NH2 | aminoethoxy)] and |
| | | | C(O) of Glu form a |
| | | 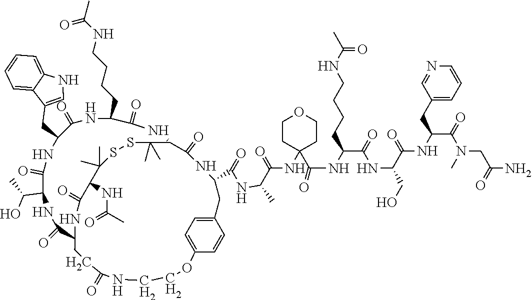 [SMILES: CC(=O)NCCCC[C@@H]1NC(=O)[C@H](Cc2c[nH]c3ccccc23)NC(=O)[C@H]([C@@H](C)O)NC(=O)[C@H]2CCC(=O)NCCOc3ccc(cc3)C[C@@H](C(=O)N[C@@H](C)C(=O)NC3(C(=O)N[C@@H](CCCCNC(C)=O)C(=O)N[C@@H](CO)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | lactam Isomer 1 |
| 26 | 9 | Ac-[Pen]-E-T-W-[Lys(Ac)]-[Pen]-K-[2Nal]-[THP]- | NH of Lys and |
| | | [Lys(Ac)]-S-[3Pal]-[Sarc]-NH2 | C(O) of Glu form a |
| | | | lactam |
| | | 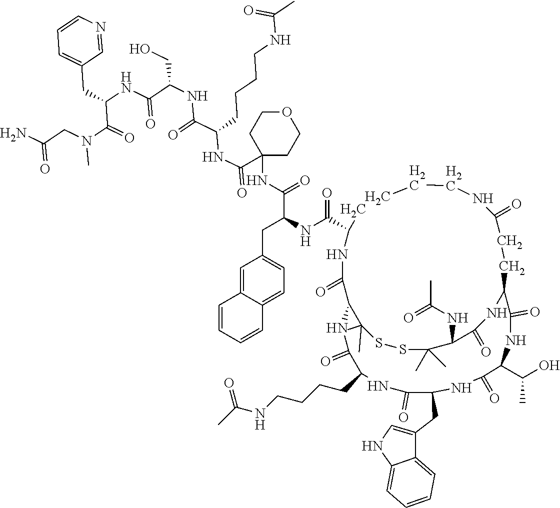 [SMILES: CCCNC(=O)CC[C@@H]1NC(=O)[C@@H](NC(C)=O)C(C)(C)SSC(C)(C)[C@@H](C(=O)N[C@@H](C)C(=O)N[C@@H](Cc2ccc3ccccc3c2)C(=O)NC2(C(=O)N[C@@H](CCCCNC(C)=O)C(=O)N[C@@H](CO)C(=O)N[C@@H](Cc3cccnc3)C(=O)N(C)CC(N)=O)CCOCC2)NC(=O)[C@H](CCCCNC(C)=O)NC(=O)[C@H](Cc2c[nH]c3ccccc23)NC(=O)[C@H]([C@@H](C)O)NC1=O] | |
| 27 | 10 | Ac-[Pen]-E-T-W-A-[Pen]-[Phe(4-(2-aminoethoxy))]- | NH of Phe[4-(2- |
| | | [2Nal]-[THP]-[Lys(Ac)]-S-[3Pal]-[Sarc]-NH2 | aminoethoxy)] and |
| | | | C(O) of Glu form a |
| | | 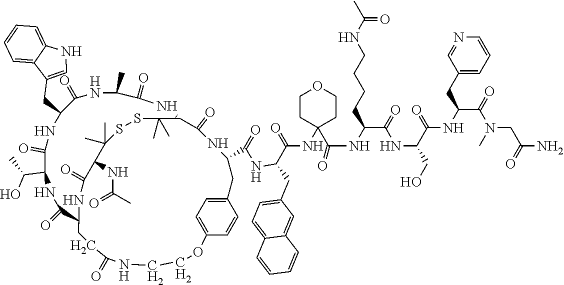 [SMILES: CC(=O)NCCCC[C@H](NC(=O)C1(NC(=O)[C@H](Cc2ccc3ccccc3c2)NC(=O)[C@@H]2Cc3ccc(cc3)OCCNC(=O)CC[C@H]3NC(=O)[C@@H](NC(C)=O)C(C)(C)SSC(C)(C)[C@H](NC(=O)[C@H](C)NC(=O)[C@H](Cc4c[nH]c5ccccc45)NC(=O)[C@H]([C@@H](C)O)NC3=O)C(=O)N2)CCOCC1)C(=O)N[C@@H](CO)C(=O)N[C@@H](Cc1cccnc1)C(=O)N(C)CC(N)=O] | lactam Isomer 1 |
| 28 | 11 | Ac-[Pen]-E-A-W-[Lys(Ac)]-[Pen]-[Phe(4-(2- | —NH of Phe[4-(2- |
| | | aminoethoxy))]-[2Nal]-[THP]-[Lys(Ac)]-S-[3Pal]-[Sarc]- | aminoethoxy)] and |
| | | NH2 | C(O) of Glu form a |
| | | | lactam |
| | | 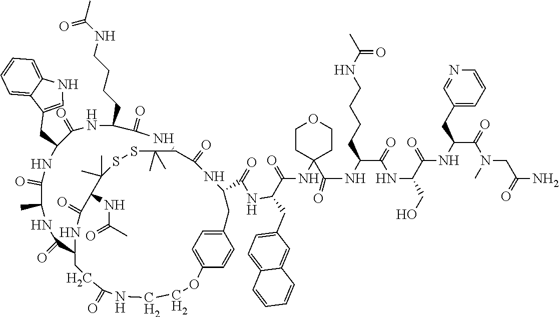 [SMILES: CC(=O)NCCCC[C@@H]1NC(=O)[C@H](Cc2c[nH]c3ccccc23)NC(=O)[C@H](C)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CO)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | |
| 29 | 12 | Ac-[Abu]-E-T-W-[Lys(Ac)]-C-[Phe(4-(2-aminoethoxy))]- | —NH of Phe[4-(2- |
| | | [2Nal]-[THP]-[Lys(Ac)]-S-[3Pal]-[Sarc]-NH2 | aminoethoxy)] and |
| | | | C(O) of Glu form a |
| | | 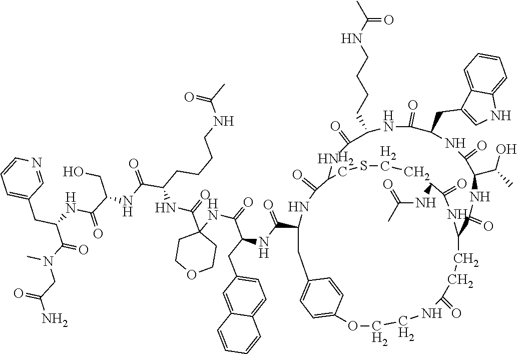 [SMILES: CC(=O)NCCCC[C@@H]1NC(=O)[C@@H](Cc2c[nH]c3ccccc23)NC(=O)[C@@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CO)C(=O)N[C@@H](Cc4cccnc4)C(=O)N(C)CC(N)=O)CCOCC3)NC(=O)C(CSCC[C@@H](NC(C)=O)C(=O)N2)NC1=O] | lactam |
| 30 | 13 | Ac-[Abu]-E-T-W-[Lys(Ac)]-C-K-[2Nal]-[THP]- | NH of Lys and |
| | | [Lys(Ac)]-S-[3Pal]-[Sarc]-NH2 | C(O) of Glu form a |
| | | | lactam |
| | | 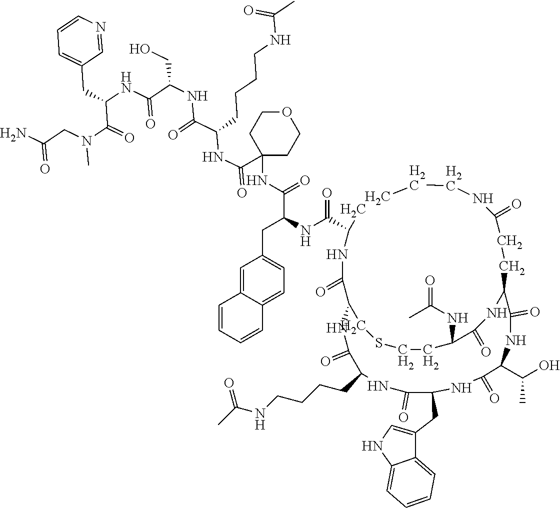 [SMILES: CCCNC(=O)CC[C@@H]1NC(=O)[C@@H](NC(C)=O)CCSC[C@@H](C(=O)N[C@@H](C)C(=O)N[C@@H](Cc2ccc3ccccc3c2)C(=O)NC2(C(=O)N[C@@H](CCCCNC(C)=O)C(=O)N[C@@H](CO)C(=O)N[C@@H](Cc3cccnc3)C(=O)N(C)CC(N)=O)CCOCC2)NC(=O)[C@H](CCCCNC(C)=O)NC(=O)[C@H](Cc2c[nH]c3ccccc23)NC(=O)[C@H]([C@@H](C)O)NC1=O] | |
| 31 | 14 | Ac-[Pen]-K-T-W-[Lys(Ac)]-[Pen]-E-[2Nal]-[THP]- | NH of Lys and |
| | | [Lys(Ac)]-S-[3Pal]-[Sarc]-NH2 | C(O) of Glu form a |
| | | | lactam |
| | | 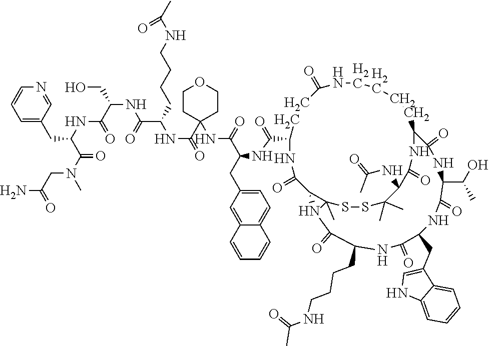 [SMILES: CC(=O)NCCCC[C@@H]1NC(=O)[C@H](Cc2c[nH]c3ccccc23)NC(=O)[C@H]([C@@H](C)O)NC(=O)[C@H](CCCCNC(C)=O)NC(=O)[C@@H](NC(C)=O)C(C)(C)SSC(C)(C)[C@@H](C(=O)N[C@@H](C)C(=O)N[C@@H](Cc2ccc3ccccc3c2)C(=O)NC2(C(=O)N[C@@H](CCCCNC(C)=O)C(=O)N[C@@H](CO)C(=O)N[C@@H](Cc3cccnc3)C(=O)N(C)CC(N)=O)CCOCC2)NC1=O] | |
| 32 | 15 | Ac-[Abu]-K-T-W-[Lys(Ac)]-C-E-[2Nal]-[THP]- | NH of Lys and |
| | | [Lys(Ac)]-S-[3Pal]-[Sarc]-NH2 | C(O) of Glu form a |
| | | | lactam |
| | | 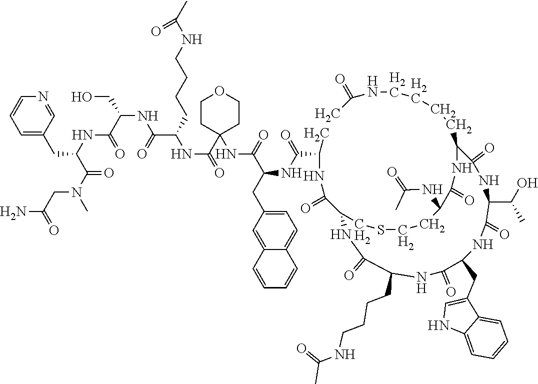 [SMILES: CC(=O)NCCCC[C@@H]1NC(=O)[C@H](Cc2c[nH]c3ccccc23)NC(=O)[C@H]([C@@H](C)O)NC(=O)[C@H](CCCCNC(C)=O)NC(=O)[C@@H](NC(C)=O)CCSC[C@@H](C(=O)N[C@@H](C)C(=O)N[C@@H](Cc2ccc3ccccc3c2)C(=O)NC2(C(=O)N[C@@H](CCCCNC(C)=O)C(=O)N[C@@H](CO)C(=O)N[C@@H](Cc3cccnc3)C(=O)N(C)CC(N)=O)CCOCC2)NC1=O] | |
| 33 | 16 | Ac-[Pen]-E-T-[W(7-Me)]-[Lys(Ac)]-[Pen]-[Phe(4-(2- | —NH of Phe[4-(2- |
| | | aminoethoxy))]-[2Nal]-[THP]-[Lys(Ac)]-NH2 | aminoethoxy)] and |
| | | | C(O) of Glu form a |
| | | 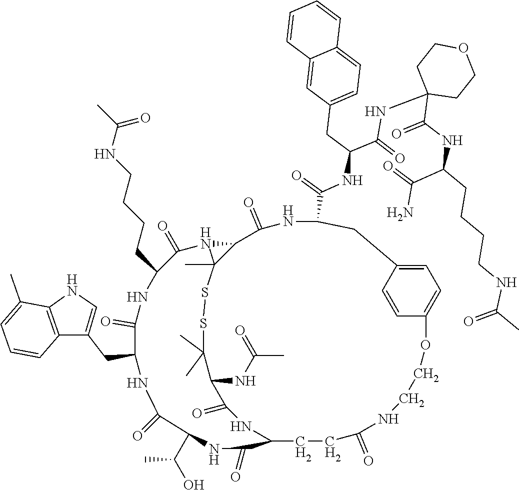 [SMILES: CC(=O)NCCCC[C@H](NC(=O)C1(NC(=O)[C@H](Cc2ccc3ccccc3c2)NC(=O)[C@@H]2Cc3ccc(cc3)OCCNC(=O)CC[C@@H]3NC(=O)[C@@H](NC(C)=O)C(C)(C)SSC(C)(C)[C@H](NC(=O)[C@H](CCCCNC(C)=O)NC(=O)[C@H](Cc4c[nH]c5c(C)cccc45)NC(=O)[C@H]([C@@H](C)O)NC3=O)C(=O)N2)CCOCC1)C(N)=O] | lactam |
| 34 | 17 | Ac-[Pen]-E-T-[W(7-Me)]-[Lys(Ac)]-[Pen]-[Phe(4-(2- | —NH of Phe[4-(2- |
| | | aminoethoxy))]-[2Nal]-[THP]-NH2 | aminoethoxy)] and |
| | | | C(O) of Glu form a |
| | | 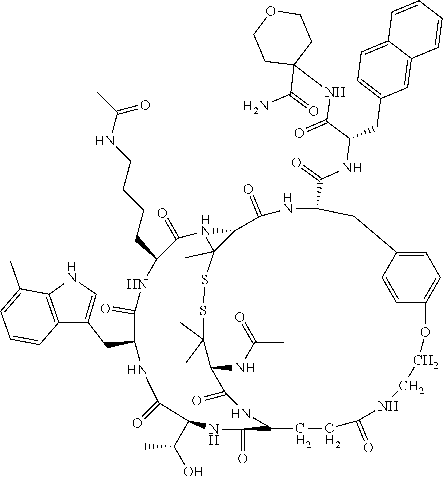 | lactam |
| 35 | 18 | Ac-[(D)Orn]-[Pen]-N-T-[W(7-Me)]-Q-[Pen]-[Phe(4-(2- | NH of (D)Orn and |
| | | aminoethoxy))]-[2Nal]-[THP]-E-N-[THP]-NH2 | C(O) of Glu form a |
| | | | lactam |
| | | 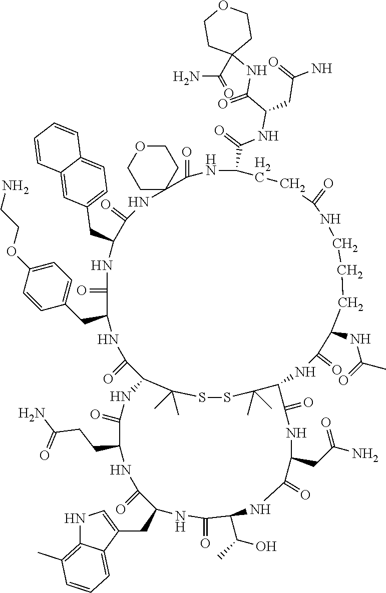 [SMILES: CC(=O)N[C@@H]1CCCNC(=O)CC[C@@H](C(=O)N[C@@H](CC(N)=O)C(=O)NC2(C(N)=O)CCOCC2)NC(=O)C2(CCOCC2)NC(=O)[C@H](Cc2ccc3ccccc3c2)NC(=O)[C@H](Cc2ccc(OCCN)cc2)NC(=O)[C@H]2NC(=O)[C@H](CCC(N)=O)NC(=O)[C@H](Cc3c[nH]c4c(C)cccc34)NC(=O)[C@H]([C@@H](C)O)NC(=O)[C@H](CC(N)=O)NC(=O)[C@@H](NC1=O)C(C)(C)SSC2(C)C] | |
| 36 | 19 | Ac-[(D)Orn]-[Pen]-N-T-[W(7-Me)]-[Lys(Ac)]-[Pen]- | NH of (D)Orn and |
| | | [Phe(4-(2-aminoethoxy))]-[2Nal]-[aMeLys]-E-N-[(D)Lys]- | C(O) of Glu form a |
| | | [(D)NMeTyr]-NH2 | lactam |
| | | 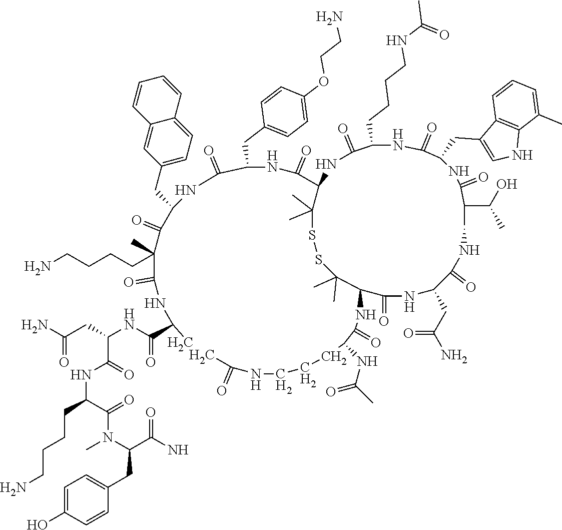 | |
| 37 | 20 | Ac-[(D)Orn]-[Pen]-N-T-[W(7-Me)]-[Lys(Ac)]-[Pen]- | NH of (D)Orn and |
| | | [Phe(4-(2-aminoethoxy))]-[2Nal]-[THP]-E-N-[THP]—NH2 | C(O) of Glu form a |
| | | | lactam |
| | | 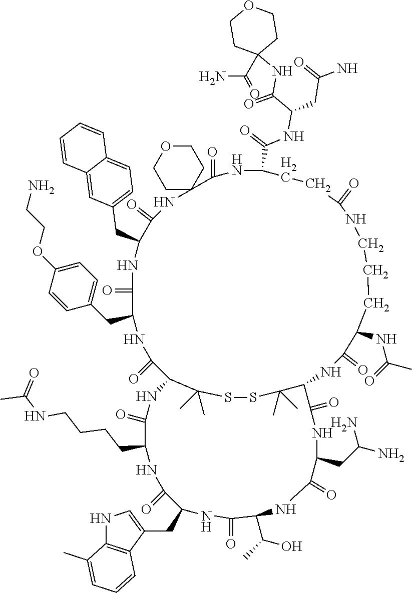 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)N)NC(=O)[C@H]2NC(=O)[C@H](NC(C)=O)CCCNC(=O)CC[C@@H](C(=O)N[C@@H](CC(N)=O)C(=O)NC3(C(N)=O)CCOCC3)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C] | |
| 38 | 21 | [7AminoHeptanoicAcid]-[Pen]-N-T-W-[Lys(Ac)]-[Pen]- | NH of |
| | | [Phe(4-(2-aminoethoxy))]-[2Nal]-[THP]-E-N-[3Pal]- | 7aminoheptanoic |
| | | [Sarc]-NH2 | acid and C(O) of |
| | | | Glu form a lactam |
| | | 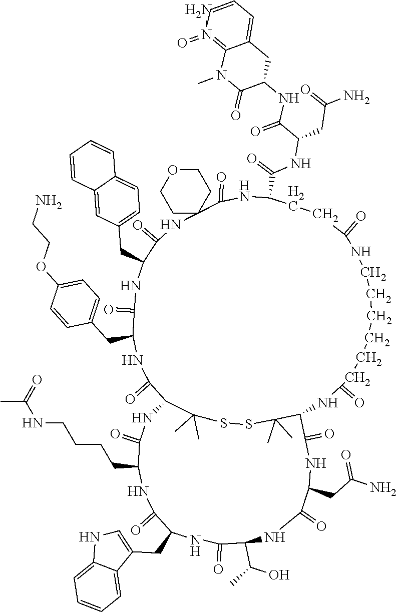 | |
| 39 | 22 | [7AminoHeptanoicAcid]-[Pen]-N-T-[W(7-Me)]- | NH of |
| | | [Lys(Ac)]-[Pen]-[Phe(4-(2-aminoethoxy))]-[2Nal]-[THP]- | 7aminoheptanoic |
| | | E-N-[3Pal]-[Sarc]-NH2 | acid and C(O) of |
| | | | Glu form a lactam |
| | | 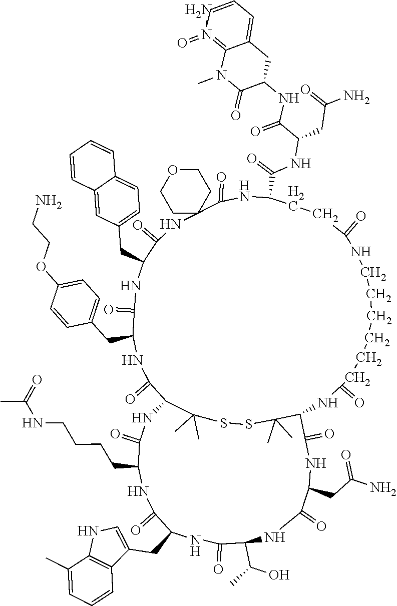 | |
| 40 | 23 | [Ahx]-[Pen]-N-T-[W(7-Me)]-[Lys(Ac)]-[Pen]-[Phe(4-(2- | NH of Ahx and |
| | | aminoethoxy))]-[2Nal]-[THP]-E-N-[THP]-NH2 | C(O) of Glu form a |
| | | | lactam |
| | | 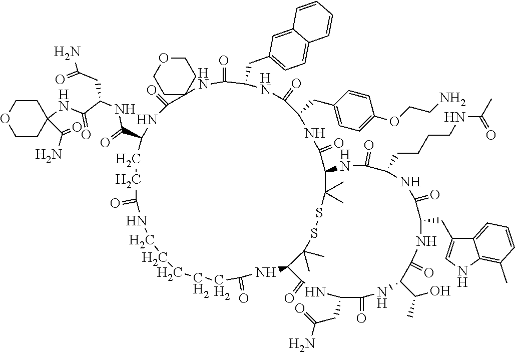 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)CCCCCNC(=O)CC[C@@H](C(=O)N[C@@H](CC(N)=O)C(=O)NC3(C(N)=O)CCOCC3)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C] | |
| 41 | 24 | [6AminoHexanoicAcid]-[Pen]-N-T-[W(7-Me)]-[Lys(Ac)]- | —NH of Phe[4-(2- |
| | | [Pen]-[Phe(4-(2-aminoethoxy))]-[2Nal]-[THP]-E-N- | aminoethoxy)] and |
| | | [THP]-NH2 | C(O) of Glu form a |
| | | | lactam |
| | | 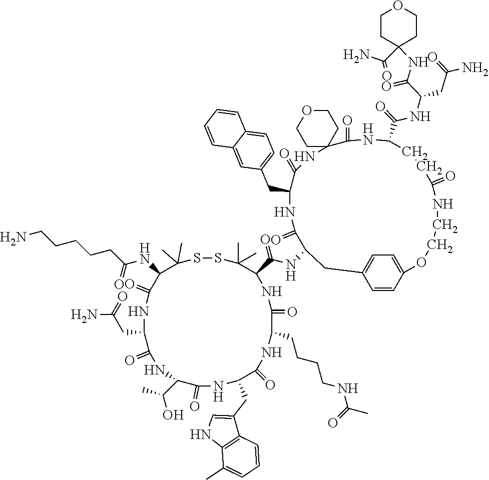 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCCCCN)C(C)(C)SSC(C)(C)[C@@H](C(=O)N[C@H]2Cc3ccc(cc3)OCCNC(=O)CC[C@@H](C(=O)N[C@@H](CC(N)=O)C(=O)NC3(C(N)=O)CCOCC3)NC(=O)C3(CCOCC3)NC(=O)[C@H](Cc3ccc4ccccc4c3)NC2=O)NC1=O] | |
| 42 | 25 | [7AminoHeptanoicAcid]-[Pen]-N-T-[W(7-Me)]- | NH of |
| | | [Lys(Ac)]-[Pen]-[Phe(4-(2-aminoethoxy))]-[2Nal]-[THP]- | 7aminoheptanoic |
| | | E-N-[(D)NMeTyr]-NH2 | acid and C(O) of |
| | | | Glu form a lactam |
| | | 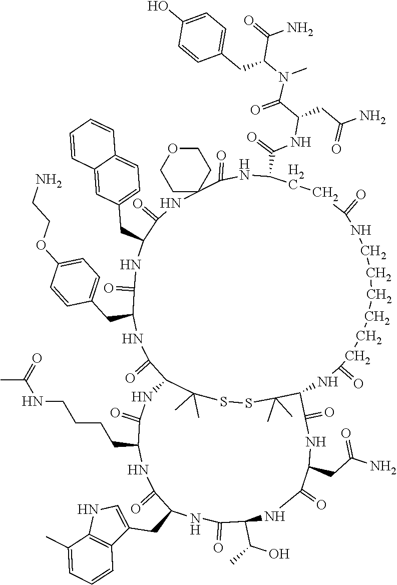 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)CCCCCCNC(=O)CC[C@@H](C(=O)N[C@@H](CC(N)=O)C(=O)N(C)[C@H](Cc3ccc(O)cc3)C(N)=O)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C] | |

wherein Pen-Pen form a disulfide bond, or Abu-Cys form a thioether bond.

TABLE 1C
Compounds

| SEQ | | | |
|---|---|---|---|
| ID. | Cmpd. | Sequence/Structure | Note |
| 43 | 26 | [Ahx]-[Abu]-N-T-W-Q-C-[Phe(4-(2-aminoethoxy))]- | NH of Ahx and |
| | | [2Nal]-[THP]-E-N-[3Pal]-[Sarc]-NH2 | C(O) of Glu form a |
| | | | lactam |
| | | 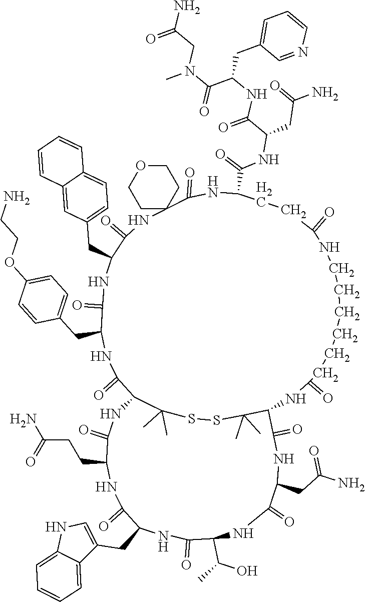 [SMILES: C[C@@H](O)[C@@H]1NC(=O)[C@H](CC(N)=O)NC(=O)[C@H]2NC(=O)CCCCCCNC(=O)CC[C@@H](C(=O)N[C@@H](CC(N)=O)C(=O)N[C@@H](Cc3cccnc3)C(=O)N(C)CC(N)=O)NC(=O)C3(CCOCC3)NC(=O)[C@H](Cc3ccc4ccccc4c3)NC(=O)[C@H](Cc3ccc(OCCN)cc3)NC(=O)[C@@H](NC(=O)[C@H](CCC(N)=O)NC(=O)[C@H](Cc3c[nH]c4ccccc34)NC1=O)C(C)(C)SSC2(C)C] | |
| 44 | 27 | Ac-[Orn]-[Abu]-Q-T-W-Q-C-[Phe(4-(2-aminoethoxy))]- | #N/A |
| | | [2Nal]-[THP]-E-N-[aMePhe]-NH2 | |
| | | 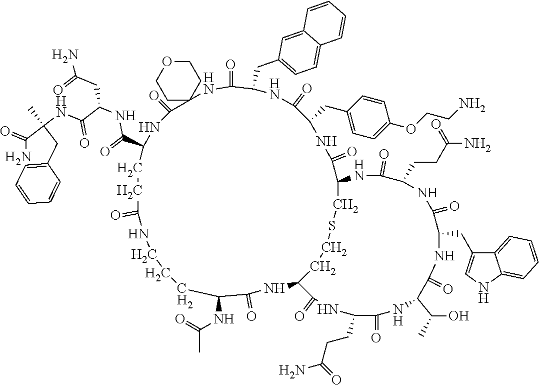 [SMILES: CC(=O)N[C@H]1CCCNC(=O)CC[C@@H](C(=O)N[C@@H](CC(N)=O)C(=O)N[C@@](C)(Cc2ccccc2)C(N)=O)NC(=O)C2(CCOCC2)NC(=O)[C@H](Cc2ccc3ccccc3c2)NC(=O)[C@H](Cc2ccc(OCCN)cc2)NC(=O)[C@@H]2CSCC[C@H](NC1=O)C(=O)N[C@@H](CCC(N)=O)C(=O)N[C@@H]([C@@H](C)O)C(=O)N[C@@H](Cc1c[nH]c3ccccc13)C(=O)N[C@@H](CCC(N)=O)C(=O)N2] | |
| 45 | 28 | [Ahx]-[Abu]-N-T-W-Q-C-[Phe(4-(2-aminoethoxy))]- | NH of Ahx and |
| | | [2Nal]-[aMeLys]-E-N-[3Pal]-[Sarc]-NH2 | C(O) of Glu form a |
| | | | lactam |
| | | 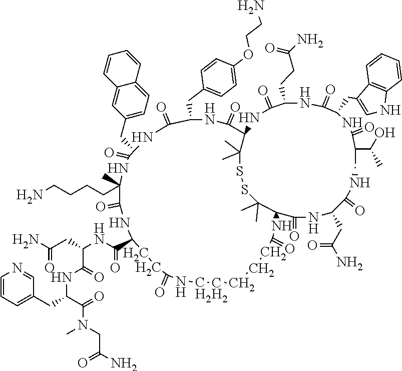 | |
| 46 | 29 | [5AminoPentanoic Acid]-[Abu]-N-T-W-Q-C-[Phe(4-(2- | NH of |
| | | aminoethoxy))]-[2Nal]-[THP]-E-N-[THP]-NH2 | 5aminopentanoic |
| | | | acid and C(O) of |
| | | 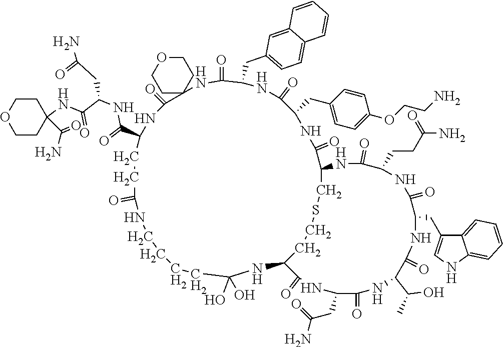 [SMILES: C[C@@H](O)[C@@H]1NC(=O)[C@H](CC(N)=O)NC(=O)[C@@H]2CCSC[C@H](NC(=O)[C@H](CCC(N)=O)NC(=O)[C@H](Cc3c[nH]c4ccccc34)NC1=O)C(=O)N[C@@H](Cc1ccc(OCCN)cc1)C(=O)N[C@@H](Cc1ccc3ccccc3c1)C(=O)NC1(CCOCC1)C(=O)N[C@H](C(=O)N[C@@H](CC(N)=O)C(=O)NC1(C(N)=O)CCOCC1)CCC(=O)NCCCCC(O)(O)N2] | Glu form a lactam |
| 47 | 30 | [7AminoHeptanoicAcid]-[Pen]-N-T-[W(7-Me)]- | NH of |
| | | [Lys(Ac)]-[Pen]-[Phe(4-(2-aminoethoxy))]-[2Nal]-[THP]- | 7aminoheptanoic |
| | | E-N-[THP]-NH2 | acid and C(O) of |
| | | | Glu form a lactam |
| | | 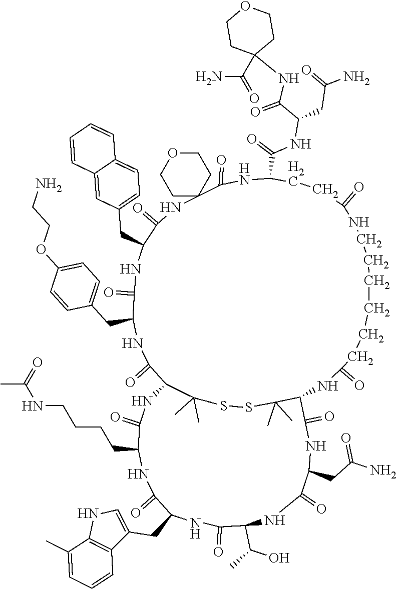 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)CCCCCCNC(=O)CC[C@@H](C(=O)N[C@@H](CC(N)=O)C(=O)NC3(C(N)=O)CCOCC3)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C] | |
| 48 | 31 | Ac-[Pen]-N-[Pro(3-OH)]-[W(7-Me)]-[O3S]-[Pen]-[Phe(4- | side chain C of |
| | | (2-aminoethoxy))]-[2Nal]-[O3S]-[Lys(Ac)]-N-[3Pal]-NH2 | (D)Ala(X8) and |
| | | | side chain C of |
| | | 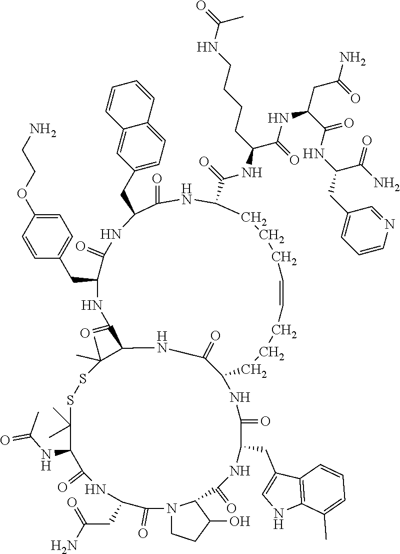 [SMILES: CC(=O)NCCCC[C@H](NC(=O)[C@@H]1CCC/C=C\CCC[C@@H]2NC(=O)[C@H](Cc3c[nH]c4c(C)cccc34)NC(=O)[C@@H]3C(O)CCN3C(=O)[C@H](CC(N)=O)NC(=O)[C@@H](NC(C)=O)C(C)(C)SSC(C)(C)[C@H](NC2=O)C(=O)N[C@@H](Cc2ccc(OCCN)cc2)C(=O)N[C@@H](Cc2ccc3ccccc3c2)C(=O)N1)C(=O)N[C@@H](CC(N)=O)C(=O)N[C@@H](Cc1cccnc1)C(N)=O] | (D)Ala(X12) cyclized via —CH2— CH2— CH═CH— CH2—CH2— Isomer 1 |
| 49 | 32 | Ac-[Pen]-N-T-[W(7-Me)]-[O3S]-[Pen]-[Phe(4-(2- | side chain C of |
| | | aminoethoxy))]-[2Nal]-[O3S]-[Lys(Ac)]-N-[3Pal]-[Sarc]-NH2 | (D)Ala(X8) and |
| | | | side chain C of |
| | | 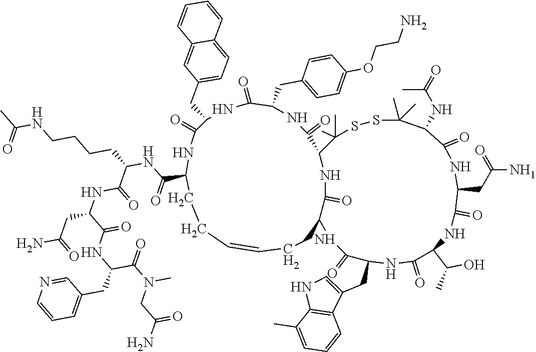 [SMILES: CC(=O)NCCCC[C@H](NC(=O)[C@@H]1CC/C=C\CC[C@@H]2NC(=O)[C@H](Cc3c[nH]c4c(C)cccc34)NC(=O)[C@H]([C@@H](C)O)NC(=O)[C@H](CC(N)=O)NC(=O)[C@@H](NC(C)=O)C(C)(C)SSC(C)(C)[C@H](NC2=O)C(=O)N[C@@H](Cc2ccc(OCCN)cc2)C(=O)N[C@@H](Cc2ccc3ccccc3c2)C(=O)N1)C(=O)N[C@@H](CC(N)=O)C(=O)N[C@@H](Cc1cccnc1)C(=O)N(C)CC(N)=O] | (D)Ala(X12) cyclized via —CH2— CH2—CH═CH— CH2—CH2— Isomer 2 |
| 50 | 33 | [7AminoHeptanoicAcid]-[Pen]-N-T-[W(7-Me)]- | NH of |
| | | [Lys(Ac)]-[Pen]-[Phe(4-(2-aminoethoxy))]-[2Nal]-[THP]- | 7aminoheptanoic |
| | | E-N-[3Pal]-NH2 | acid and C(O) of |
| | | | Glu form a lactam |
| | | 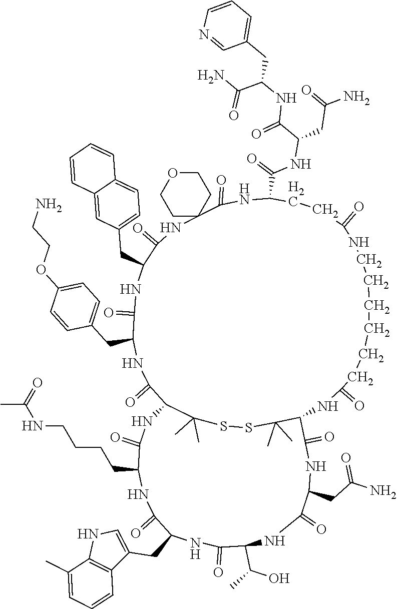 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)CCCCCCNC(=O)CC[C@@H](C(=O)N[C@@H](CC(N)=O)C(=O)N[C@@H](Cc3cccnc3)C(N)=O)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C] | |
| 51 | 34 | Ac-[Pen]-N-T-[W(7-Me)]-[Lys(Ac)]-[Pen]-[Phe(4-(2- | #N/A |
| | | aminoethoxy))]-[3Quin]-[THP]-E-N—H-[Sarc]-[NH2]- | |
| | | [PEG4] | |
| | | 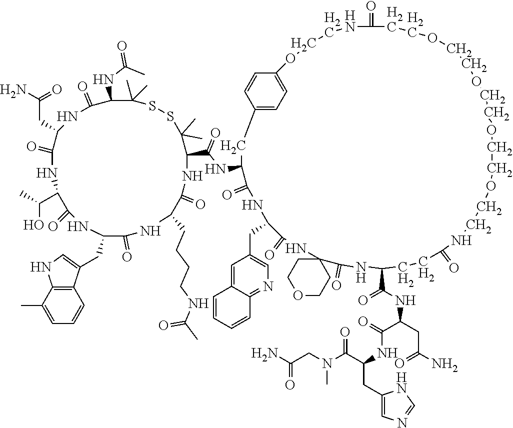 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@H]2Cc3ccc(cc3)OCCNC(=O)CCOCCOCCOCCOCCNC(=O)CC[C@@H](C(=O)N[C@@H](CC(N)=O)C(=O)N[C@@H](Cc3cnc[nH]3)C(=O)N(C)CC(N)=O)NC(=O)C3(CCOCC3)NC(=O)[C@H](Cc3cnc4ccccc4c3)NC2=O)NC1=O] | |
| 52 | 35 | [Gaba]-[(D)Arg]-[Pen]-N-T-[W(7-Me)]-[Lys(Ac)]-[Pen]- | —NH of Phe[4-(2- |
| | | [Phe(4-(2-aminoethoxy))]-[2Nal]-[THP]-E-N-[3Pal]- | aminoethoxy)] and |
| | | [Sarc]-NH2 | C(O) of Glu form a |
| | | | lactam |
| | | 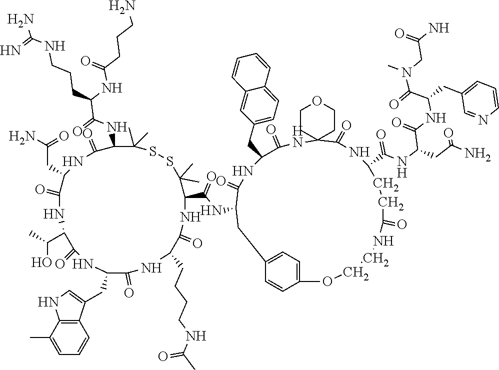 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCCN)C(C)(C)SSC(C)(C)[C@@H](C(=O)N[C@H]2Cc3ccc(cc3)OCCNC(=O)CC[C@@H](C(=O)N[C@@H](CC(N)=O)C(=O)N[C@@H](Cc3cccnc3)C(=O)N(C)CC(N)=O)NC(=O)C3(CCOCC3)NC(=O)[C@H](Cc3ccc4ccccc4c3)NC2=O)NC1=O] | |
| 53 | 36 | Ac-[(D)Lys]-[Pen]-E-T-[W(7-Me)]-[Lys(Ac)]-[Pen]- | NH of (D)Lys and |
| | | [Phe(4-(2-aminoethoxy))]-[2Nal]-[THP]-[Lys(Ac)]-N- | C(O) of Glu (X17) |
| | | [3Pal]-[Sarc]-E-NH2 | form a lactam; and |
| | | | NH of (Phe2ae) |
| | | 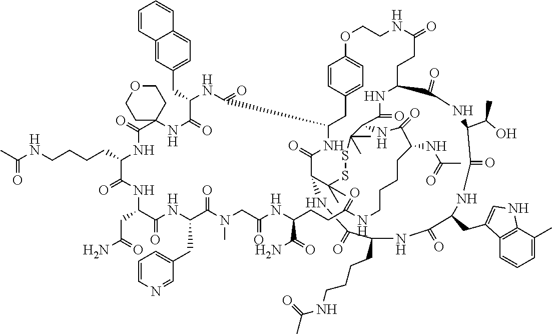 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@@H]3NC(=O)[C@@H](NC1=O)C(C)(C)SSC(C)(C)[C@H](NC(=O)[C@H](NC(C)=O)CCCCNC(=O)CC[C@@H](C(N)=O)NC(=O)CN(C)C(=O)[C@H](Cc1cccnc1)NC(=O)[C@H](CC(N)=O)NC(=O)[C@H](CCCCNC(C)=O)NC(=O)C1(CCOCC1)NC(=O)[C@H](Cc1ccc4ccccc4c1)NC3=O)C(=O)N2] | and C(O) of Glu (X5) form a lactam Isomer 1 |
| 54 | 37 | Ac-E-[Pen]-E-T-[W(7-Me)]-[Lys(Ac)]-[Pen]-[Phe(4-(2- | NH of (D)Lys and |
| | | aminoethoxy))]-[2Nal]-[THP]-[Lys(Ac)]-N-[3Pal]-[Sarc]- | C(O) of Glu (X3) |
| | | [(D)Lys]-NH2 | form a lactam; and |
| | | | NH of (Phe2ae) |
| | | 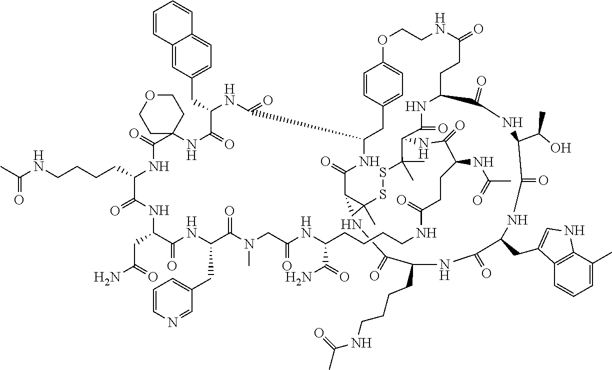 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@@H]3NC(=O)[C@@H](NC1=O)C(C)(C)SSC(C)(C)[C@H](NC(=O)[C@@H](NC(C)=O)CCC(=O)NCCCC[C@H](C(N)=O)NC(=O)CN(C)C(=O)[C@H](Cc1cccnc1)NC(=O)[C@H](CC(N)=O)NC(=O)[C@H](CCCCNC(C)=O)NC(=O)C1(CCOCC1)NC(=O)[C@H](Cc1ccc4ccccc4c1)NC3=O)C(=O)N2] | and C(O) of Glu (X5) form a lactam Isomer 1 |
| 55 | 38 | Ac-E-A-E-T-[W(7-Me)]-[Lys(Ac)]-A-[Phe(4-(2- | —NH of |
| | | aminoethoxy))]-[2Nal]-[THP]-[Lys(Ac)]-N-[3Pal]- | Phe(4(2ae)) and |
| | | [(D)Lys]-NH2 | C(O) of Glu (X3) |
| | | | form lactam; and - |
| | | 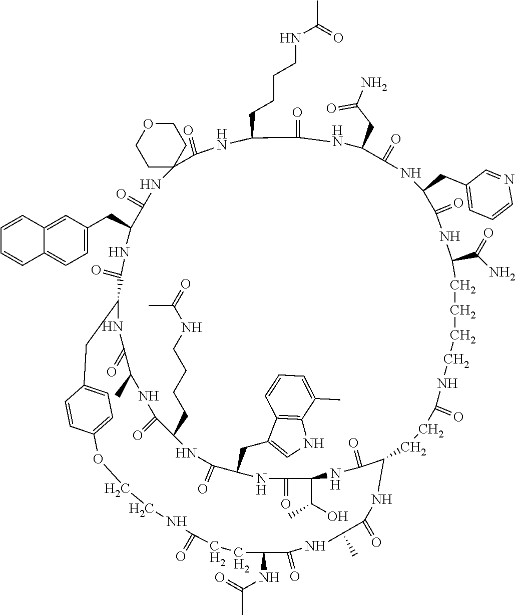 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCCC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC(=O)[C@@H](Cc3ccc(cc3)OCCNC(=O)CC[C@@H](NC(C)=O)C(=O)N[C@H](C)C(=O)N2)NC(=O)[C@H](C)NC1=O] | NH of (D)Lys and C(O) of Glu (X5) form a lactam |
| 56 | 39 | E-T-[W(7-Me)]-[Lys(Ac)]-A-[Phe(4-(2-aminoethoxy))]- | The alpha amino |
| | | [2Nal]-[THP]-[Lys(Ac)]-[3Pal] | group of E forms |
| | | | an amide with the |
| | | 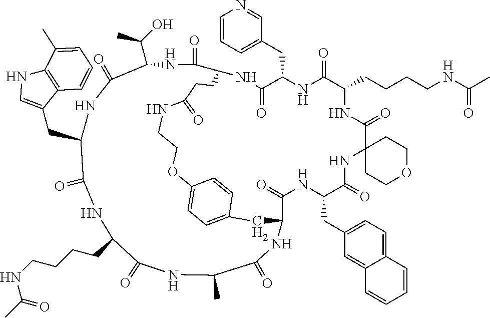 [SMILES: CC(=O)NCCCC[C@@H]1NC(=O)C2(CCOCC2)NC(=O)[C@H](Cc2ccc3ccccc3c2)NC(=O)[C@H]2Cc3ccc(cc3)OCCNC(=O)CC[C@H](NC(=O)[C@H](Cc3cccnc3)NC1=O)C(=O)N[C@@H]([C@@H](C)O)C(=O)N[C@H](Cc1c[nH]c3c(C)cccc13)C(=O)N[C@H](CCCCNC(C)=O)C(=O)N[C@H](C)C(=O)N2] | carboxyl terminus of Pal and the side chain carboxyl of E forms an amide with Phe(4-(2- aminoethoxy) |
| 57 | 40 | E-T-[W(7-Me)]-[Lys(Ac)]-A-[Phe(4-(2-aminoethoxy))]- | The alpha amino |
| | | [2Nal]-[THP]-[3Pal] | group of E forms |
| | | | an amide with the |
| | | 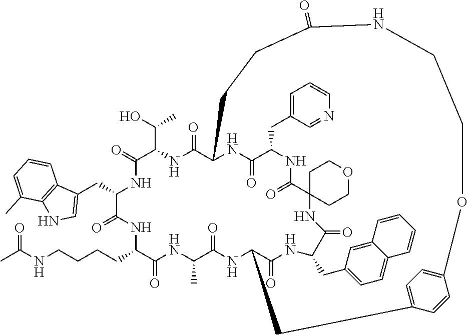 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@H](NC(=O)[C@H](C)NC1=O)C(=O)N[C@@H](Cc1ccc3ccccc3c1)C(=O)NC1(CCOCC1)C(=O)N[C@@H](Cc1cccnc1)C(=O)N2] | carboxyl terminus of Pal and the side chain carboxyl of E forms an amide with Phe(4-(2- aminoethoxy) |
| 58 | 41 | Ac-[Orn]-[Pen]-N-T-[W(7-Me)]-[Lys(Ac)]-[Pen]-[Phe(4- | NH of Orn and |
| | | (2-aminoethoxy))]-[2Nal]-[THP]-E-NH2 | C(O) of Glu form a |
| | | | lactam Isomer 1 |
| | | 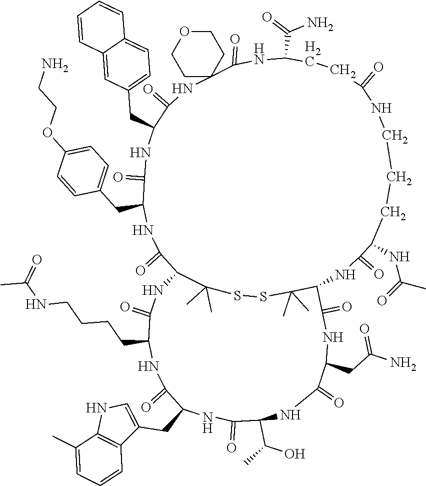 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@@H](NC(C)=O)CCCNC(=O)CC[C@@H](C(N)=O)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C] | |
| 59 | 42 | Ac-[Pen]-D-T-[W(7-Me)]-[Lys(Ac)]-[Pen]-[Phe(4-(2- | #N/A |
| | | aminoethoxy))]-[2Nal]-[THP]-[Lys(Ac)]-N—H-[Sarc]-NH2 | |
| | | 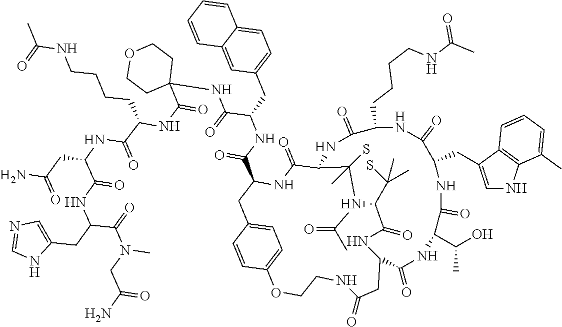 | |
| 60 | 43 | Ac-[Pen]-E-T-[W(7-Me)]-[Lys(Ac)]-[Pen]-[Phe(4-(2- | #N/A |
| | | aminoethoxy))]-[2Nal]-[THP]-[Lys(Ac)]-N—H-[Sarc]-NH2 | |
| | | 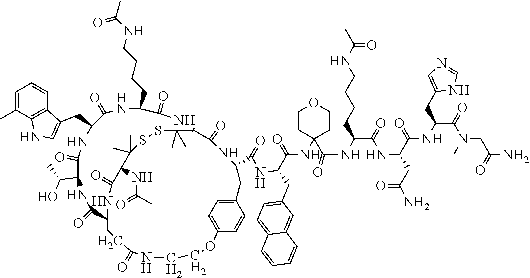 [SMILES: CC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cnc[nH]4)C(=O)N(C)CC(N)=O)CCOCC3)NC(=O)[C@@H](NC1=O)C(C)(C)SSC(C)(C)[C@H](NC(C)=O)C(=O)N2] | |
| 61 | 44 | Ac-[Dap]-[Abu]-Q-T-W-Q-C-[Phe(4-(2-aminoethoxy))]- | #N/A |
| | | [2Nal]-[THP]-E-N-[bAla]-NH2 | |
| | | 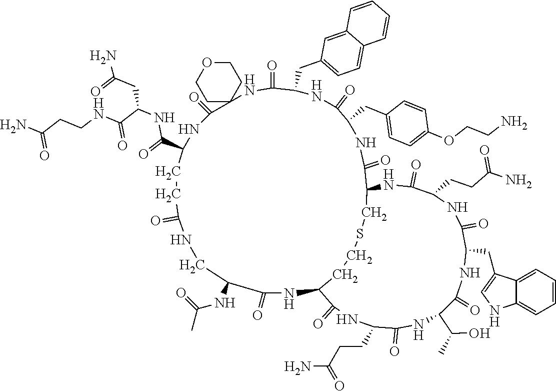 [SMILES: CC(=O)N[C@H]1CNC(=O)CC[C@@H](C(=O)N[C@@H](CC(N)=O)C(=O)NCCC(N)=O)NC(=O)C2(CCOCC2)NC(=O)[C@H](Cc2ccc3ccccc3c2)NC(=O)[C@H](Cc2ccc(OCCN)cc2)NC(=O)[C@@H]2CSCC[C@H](NC1=O)C(=O)N[C@@H](CCC(N)=O)C(=O)N[C@@H]([C@@H](C)O)C(=O)N[C@@H](Cc1c[nH]c3ccccc13)C(=O)N[C@@H](CCC(N)=O)C(=O)N2] | |
| 62 | 45 | Ac-[Pen]-[Dap]-T-[W(7-Me)]-[Lys(Ac)]-[Pen]-[Phe(4-(2- | #N/A |
| | | aminoethoxy))]-[2Nal]-[THP]-[Lys(Ac)]-N—H-[Sarc]- | |
| | | [NH2]-[PEG4DA] | |
| | | 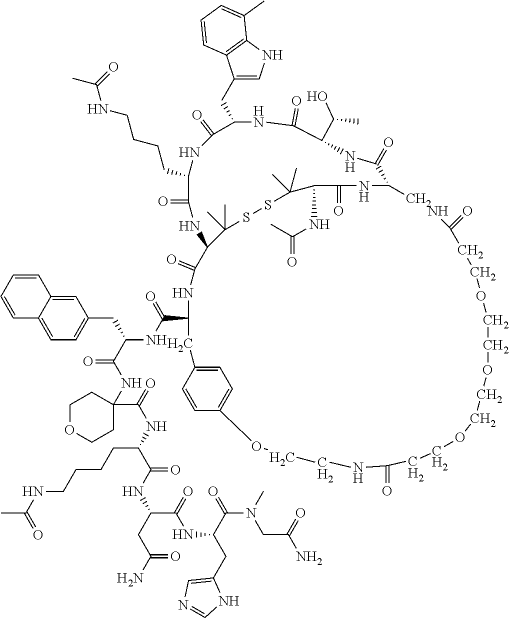 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CNC(=O)CCOCCOCCOCCC(=O)NCCOc3ccc(cc3)C[C@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cnc[nH]4)C(=O)N(C)CC(N)=O)CCOCC3)NC(=O)[C@@H](NC1=O)C(C)(C)SSC(C)(C)[C@H](NC(C)=O)C(=O)N2] | |
| 63 | 46 | Ac-[Pen]-[Dap]-T-[W(7-Me)]-[Lys(Ac)]-[Pen]-[Phe(4-(2- | #N/A |
| | | aminoethoxy))]-[2Nal]-[THP]-[Lys(Ac)]-N—H-[Sarc]- | |
| | | [NH2]-[PEG2DA] | |
| | | 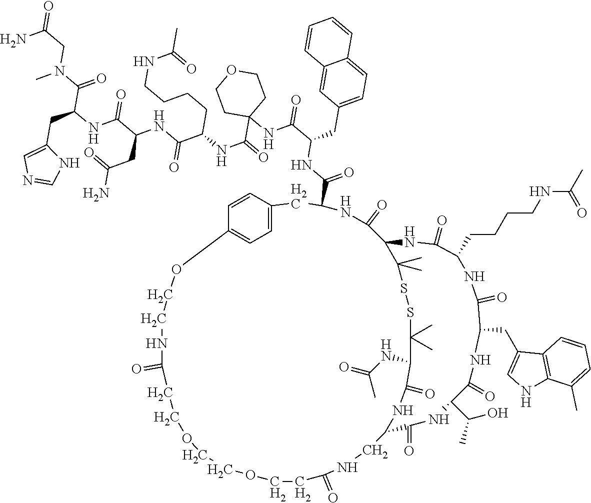 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CNC(=O)CCOCCOCCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cnc[nH]4)C(=O)N(C)CC(N)=O)CCOCC3)NC(=O)[C@@H](NC1=O)C(C)(C)SSC(C)(C)[C@H](NC(C)=O)C(=O)N2] | |
| 64 | 47 | Ac-[Pen]-[Dap]-T-[W(7-Me)]-[Lys(Ac)]-[Pen]-[Phe(4-(2- | #N/A |
| | | aminoethoxy))]-[2Nal]-[THP]-[Lys(Ac)]-N—H-[Sarc]- | |
| | | [NH2]-[Decanedioic_Acid] | |
| | | 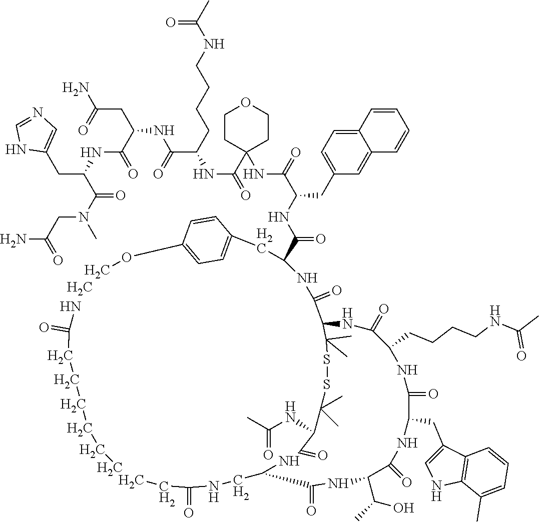 | |
| 65 | 48 | Ac-[Pen]-N-E-W-Q-[Pen]-[Phe(4-(2-aminoethoxy))]- | #N/A |
| | | [2Nal]-[aMeLeu]-[Lys(Ac)]-N N NH2 | |
| | | 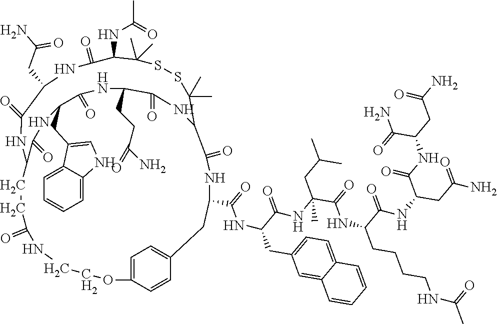 [SMILES: CC(=O)NCCCC[C@H](NC(=O)[C@](C)(CC(C)C)NC(=O)[C@H](Cc1ccc2ccccc2c1)NC(=O)[C@@H]1Cc2ccc(cc2)OCCNC(=O)CCC2NC(=O)[C@H](CC(N)=O)NC(=O)[C@@H](NC(C)=O)C(C)(C)SSC(C)(C)C(NC(=O)[C@H](CCC(N)=O)NC(=O)[C@H](Cc3c[nH]c4ccccc34)NC2=O)C(=O)N1)C(=O)N[C@@H](CC(N)=O)C(=O)N[C@@H](CC(N)=O)C(N)=O] | |
| 66 | 49 | Ac-[(D)Lys]-[Pen]-N-T-W-Q-[Pen]-[Phe(4-(2- | —NH of (D)Lys and |
| | | aminoethoxy))]-[2Nal]-[aMeLeu]-[Lys(Ac)]-N—N—[NH2]- | —NH of Phe(4- |
| | | [PEG4DA] | aminoethoxy) |
| | | | cyclized via C(O) |
| | | 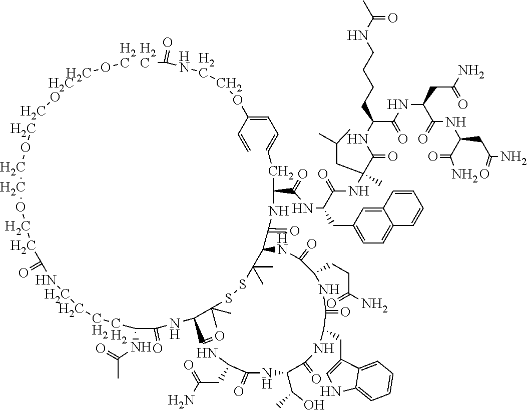 [SMILES: C=C/C1=C\C=C\C[C@H](C(=O)N[C@@H](Cc2ccc3ccccc3c2)C(=O)N[C@@](C)(CC(C)C)C(=O)N[C@@H](CCCCNC(C)=O)C(=O)N[C@@H](CC(N)=O)C(=O)N[C@@H](CC(N)=O)C(N)=O)NC(=O)[C@H]2NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@@H](NC(=O)[C@H](NC(C)=O)CCCCNC(=O)CCOCCOCCOCCOCCC(=O)NCCO1)C(C)(C)SSC2(C)C] | (CH2CH2O)4— CH2—CH2—C(O)- linker |

wherein Pen-Pen form a disulfide bond, or Abu-C form a thioether bond.

TABLE 1D
Compounds

| SEQ | | | |
|---|---|---|---|
| ID. | Cmpd. | Sequence/Structure | Note |
| 67 | 50 | Ac-[Pen]-N-K-W-Q-[Pen]-[Phe(4-(2-aminoethoxy))]- [2Nal]-[aMeLeu]-[Lys(Ac)]-N—N—[NH2]-[PEG4DA] 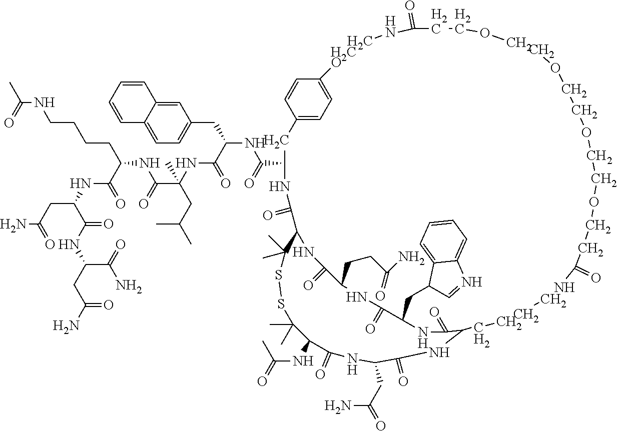 | —NH of Phe[4- (2- aminoethoxy)] and —NH of Lys cyclized via —C(O)— (CH2CH2O)4— CH2—CH2— C(O)-linker |
| 68 | 51 | Ac-[Pen]-N-T-W-Q-[Pen]-[Phe(4-(2-aminoethoxy))]- [2Nal]-[aMeLeu]-K-N—N—[NH2]-[PEG4DA] 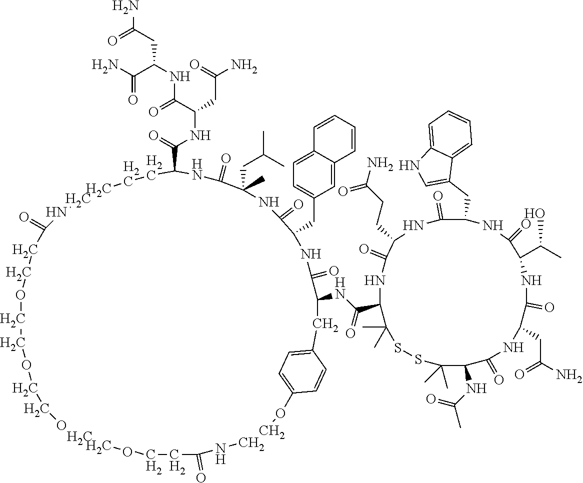 | —NH of Phe[4- (2- aminoethoxy)] and —NH of Lys cyclized via - C(O)— (CH2CH2O)4— CH2—CH2— C(O)- linker |
| 69 | 52 | [bAla]-[(D)Arg]-[Pen]-Q-T-W-Q-[Pen]-[Phe(4-(2- | —NH of bAla |
| | | aminoethoxy))]-[2Nal]-[THP]-E-N-[THP]-NH2 | and C(O) of Glu |
| | | | form a lactam |
| | | 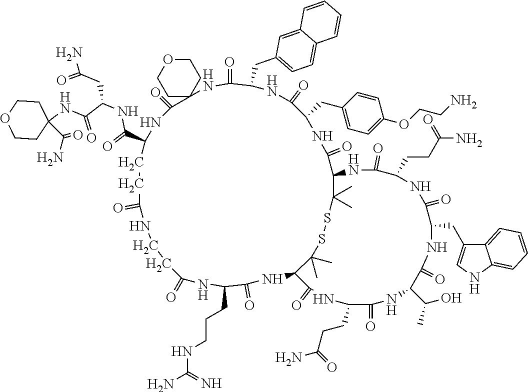 [SMILES: C[C@@H](O)[C@@H]1NC(=O)[C@H](CCC(N)=O)NC(=O)[C@H]2NC(=O)[C@@H](CCCNC(=N)N)NC(=O)CCNC(=O)CC[C@@H](C(=O)N[C@@H](CC(N)=O)C(=O)NC3(C(N)=O)CCOCC3)NC(=O)C3(CCOCC3)NC(=O)[C@H](Cc3ccc4ccccc4c3)NC(=O)[C@H](Cc3ccc(OCCN)cc3)NC(=O)[C@@H](NC(=O)[C@H](CCC(N)=O)NC(=O)[C@H](Cc3c[nH]c4ccccc34)NC1=O)C(C)(C)SSC2(C)C] | |
| 70 | 53 | [Ahx]-[Abu]-Q-T-W-Q-C-[Phe(4-(2-aminoethoxy))]- | #N/A |
| | | [2Nal]-[THP]-E-NH2 | |
| | | 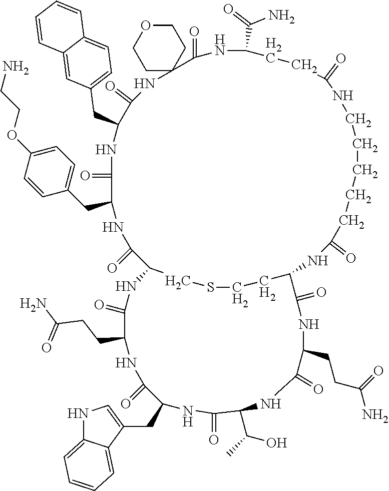 [SMILES: C[C@@H](O)[C@@H]1NC(=O)[C@H](CCC(N)=O)NC(=O)[C@@H]2CCSC[C@H](NC(=O)[C@H](CCC(N)=O)NC(=O)[C@H](Cc3c[nH]c4ccccc34)NC1=O)C(=O)N[C@@H](Cc1ccc(OCCN)cc1)C(=O)N[C@@H](Cc1ccc3ccccc3c1)C(=O)NC1(CCOCC1)C(=O)N[C@H](C(N)=O)CCC(=O)NCCCCCC(=O)N2] | |
| 71 | 54 | Ac-[Orn]-[Pen]-N-T-[W(7-Me)]-Q-[Pen]-[Phe(4-(2- | —NH of Orn and |
| | | aminoethoxy))]-[2Nal]-[THP]-E-N-[THP]-NH2 | C(O) of Glu |
| | | | form a lactam |
| | | 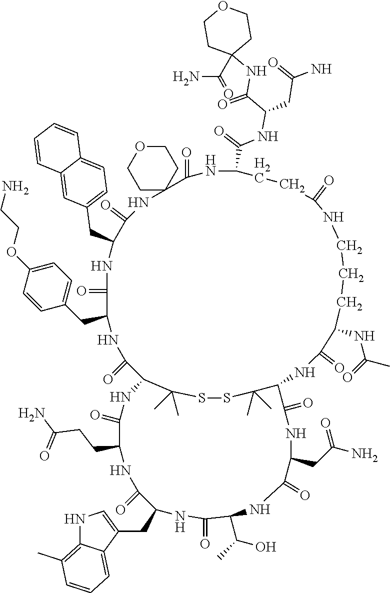 [SMILES: CC(=O)N[C@H]1CCCNC(=O)CC[C@@H](C(=O)N[C@@H](CC(N)=O)C(=O)NC2(C(N)=O)CCOCC2)NC(=O)C2(CCOCC2)NC(=O)[C@H](Cc2ccc3ccccc3c2)NC(=O)[C@H](Cc2ccc(OCCN)cc2)NC(=O)[C@H]2NC(=O)[C@H](CCC(N)=O)NC(=O)[C@H](Cc3c[nH]c4c(C)cccc34)NC(=O)[C@H]([C@@H](C)O)NC(=O)[C@H](CC(N)=O)NC(=O)[C@@H](NC1=O)C(C)(C)SSC2(C)C] | |
| 72 | 55 | Ac-[Pen]-E-T-[W(7-Me)]-[Lys(Ac)]-[Pen]-[Phe(4-(2- | —NH of Phe[4- |
| | | aminoethoxy))]-[2Nal]-[THP]-[Lys(Ac)]-N-[3Pal]-[Sarc]- | (2- |
| | | | aminoethoxy)] |
| | | 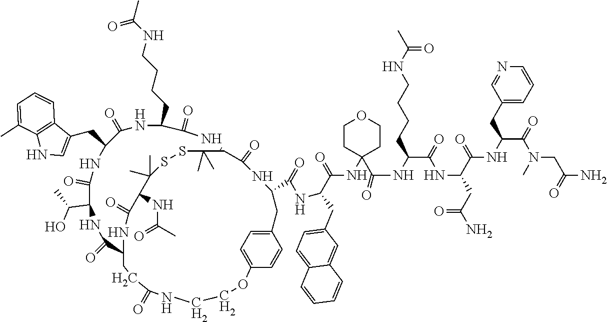 [SMILES: CC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | and C(O) of Glu form a lactam Isomer 1 |
| 73 | 56 | Ac-[Pen]-E-T-[W(7-Ph)]-[Lys(Ac)]-[Pen]-[Phe(4-(2- | —NH of Phe[4- |
| | | aminoethoxy))]-[2Nal]-[THP]-[Lys(Ac)]-N-[3Pal]-[Sarc]- | (2- |
| | | | aminoethoxy)] |
| | | 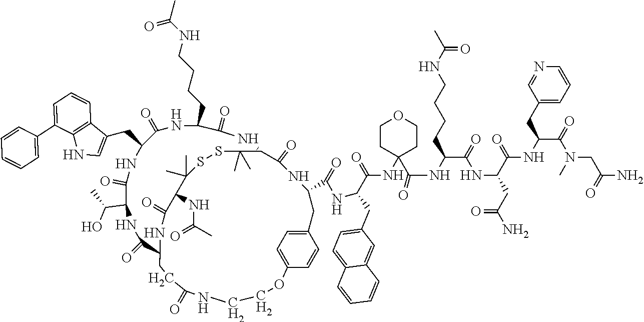 [SMILES: CC(=O)NCCCC[C@@H]1NC(=O)[C@H](Cc2c[nH]c3c(-c4ccccc4)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | and C(O) of Glu form a lactam, isomer 1 |
| 74 | 57 | Ac-[Pen]-[Dap]-T-[W(7-Me)]-[Lys(Ac)]-[Pen]-[Phe(4-(2- | —NH of Phe[4- |
| | | aminoethoxy))]-[2Nal]-[THP]-[Lys(Ac)]-N-[3Pal]-[Sarc]- | (2- |
| | | [NH2]-[PEG4DA] | aminoethoxy)] |
| | | | and —NH |
| | | 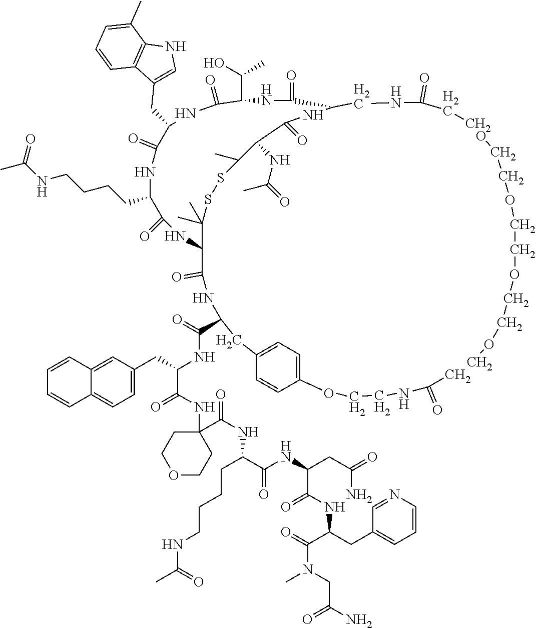 [SMILES: CCC(=O)NC[C@@H]1NC(=O)[C@@H](NC(C)=O)C(C)SSC(C)(C)[C@H](C(=O)N[C@@H](Cc2ccc(OCCNC(=O)CCOCCOCCOCCO)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC(=O)[C@H](CCCCNC(C)=O)NC(=O)[C@H](Cc2c[nH]c3c(C)cccc23)NC(=O)[C@H]([C@@H](C)O)NC1=O] | of Dap form a lactam |
| 75 | 58 | Ac-[Pen]-E-T-[W(7-Me)]-[Lys(Ac)]-[Pen]-[Phe(4-(2- | —NH of Phe[4- |
| | | aminoethoxy))]-[2Nal]-[THP]-[Lys(Ac)]-N-[(D)Leu]-NH2 | (2-aminoethoxy)] |
| | | | and C(O) of Glu |
| | | 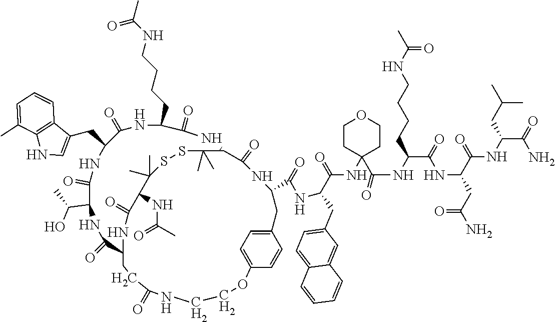 [SMILES: CC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@H](CC(C)C)C(N)=O)CCOCC3)NC(=O)[C@@H](NC1=O)C(C)(C)SSC(C)(C)[C@H](NC(C)=O)C(=O)N2] | form a lactam |
| 76 | 59 | Ac-[Pen]-E-T-[W(7-Me)]-[Lys(Ac)]-[Pen]-[Phe(4-(2- | —NH of Phe[4-(2- |
| | | aminoethoxy))]-[2Nal]-[THP]-[Lys(Ac)]-N-[Aib]-NH2 | aminoethoxy)] |
| | | | and C(O) of Glu |
| | | 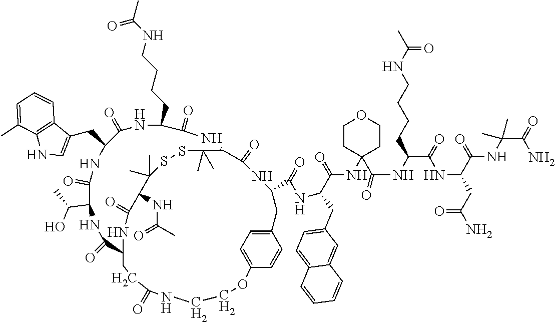 [SMILES: CC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)NC(C)(C)C(N)=O)CCOCC3)NC(=O)[C@@H](NC1=O)C(C)(C)SSC(C)(C)[C@H](NC(C)=O)C(=O)N2] | form a lactam |
| 77 | 60 | Ac-[Abu]-E-T-[W(7-Ph)]-Q-C-[Phe(4-(2-aminoethoxy))]- | —NH of Phe[4-(2- |
| | | [2Nal]-[THP]-[Lys(Ac)]-N-[3Pal]-[Sarc]-NH2 | aminoethoxy)] |
| | | | and C(O) of Glu |
| | | 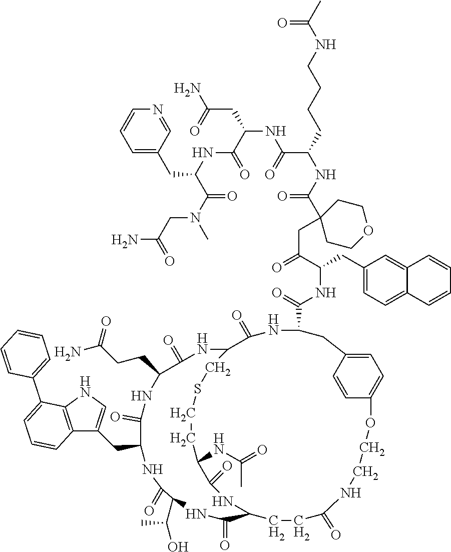 [SMILES: CC(=O)NCCCC[C@H](NC(=O)C1(CC(=O)[C@H](Cc2ccc3ccccc3c2)NC(=O)[C@@H]2Cc3ccc(cc3)OCCNC(=O)CC[C@@H]3NC(=O)[C@@H](NC(C)=O)CCSCC(NC(=O)[C@H](CCC(N)=O)NC(=O)[C@H](Cc4c[nH]c5c(-c6ccccc6)cccc45)NC(=O)[C@H]([C@@H](C)O)NC3=O)C(=O)N2)CCOCC1)C(=O)N[C@@H](CC(N)=O)C(=O)N[C@@H](Cc1cccnc1)C(=O)N(C)CC(N)=O] | form a lactam |
| 78 | 61 | Ac-[Abu]-E-T-[W(7-Me)]-Q-C-[Phe(4-(2-aminoethoxy))]- | —NH of Phe[4-(2- |
| | | [2Nal]-[THP]-[Lys(Ac)]-N-[3Pal]-[Sarc]-NH2 | aminoethoxy)] |
| | | | and C(O) of Glu |
| | | 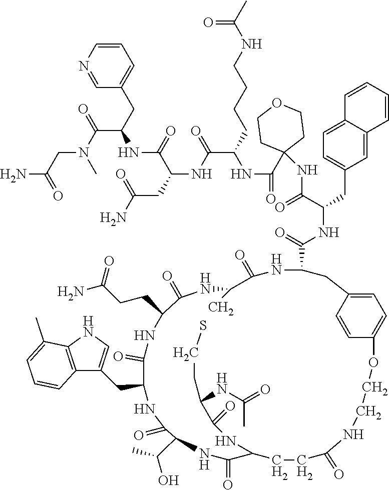 [SMILES: CC(=O)NCCCC[C@H](NC(=O)C1(NC(=O)[C@H](Cc2ccc3ccccc3c2)NC(=O)[C@@H]2Cc3ccc(cc3)OCCNC(=O)CCC(NC(=O)[C@H](CCS)NC(C)=O)C(=O)N[C@@H]([C@@H](C)O)C(=O)N[C@@H](Cc3c[nH]c4c(C)cccc34)C(=O)N[C@@H](CCC(N)=O)C(=O)N[C@@H](C)C(=O)N2)CCOCC1)C(=O)N[C@H](CC(N)=O)C(=O)N[C@H](Cc1cccnc1)C(=O)N(C)CC(N)=O] | form a lactam |
| 79 | 62 | [Ahx]-[Abu]-Q-T-W-Q-C-[Phe(4-(2-aminoethoxy))]- | #N/A |
| | | [2Nal]-[THP]-E-N-[THP]-NH2 | |
| | | 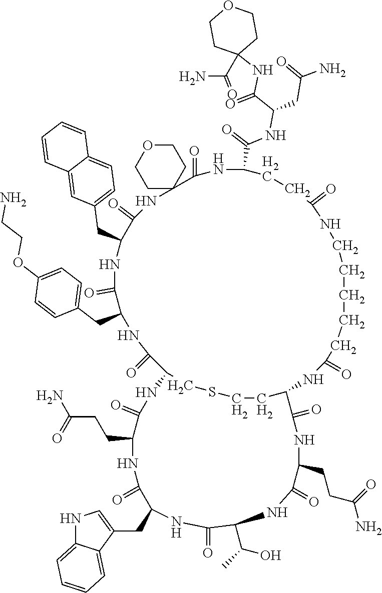 [SMILES: C[C@@H](O)[C@@H]1NC(=O)[C@H](CCC(N)=O)NC(=O)[C@@H]2CCSC[C@H](NC(=O)[C@H](CCC(N)=O)NC(=O)[C@H](Cc3c[nH]c4ccccc34)NC1=O)C(=O)N[C@@H](Cc1ccc(OCCN)cc1)C(=O)N[C@@H](Cc1ccc3ccccc3c1)C(=O)NC1(CCOCC1)C(=O)N[C@H](C(=O)N[C@@H](CC(N)=O)C(=O)NC1(C(N)=O)CCOCC1)CCC(=O)NCCCCCC(=O)N2] | |
| 80 | 63 | Ac-[Pen]-E-T-[W(7-Me)]-[Lys(Ac)]-[Pen]-[Phe(4-(2- | —NH of Phe[4-(2- |
| | | aminoethoxy))]-[2Nal]-[THP]-[Lys(Ac)]-S-[3Pal]-[Sarc]- | aminoethoxy)] |
| | | | and C(O) of Glu |
| | | 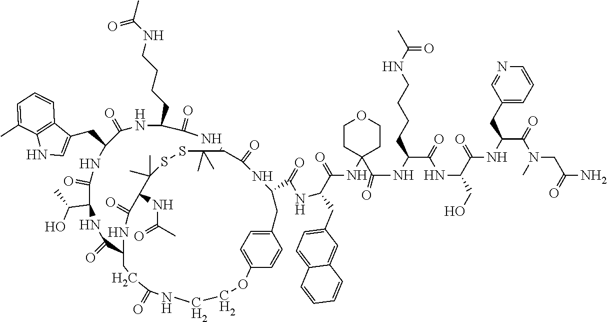 [SMILES: CC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CO)C(=O)N[C@@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | form a lactam |
| 81 | 64 | [Ahx]-[Abu]-N-T-W-Q-C-[Phe(4-(2-aminoethoxy))]- | —NH of Ahx |
| | | [2Nal]-[THP]-E-N-[THP]-NH2 | and C(O) of Glu |
| | | | form a lactam |
| | | 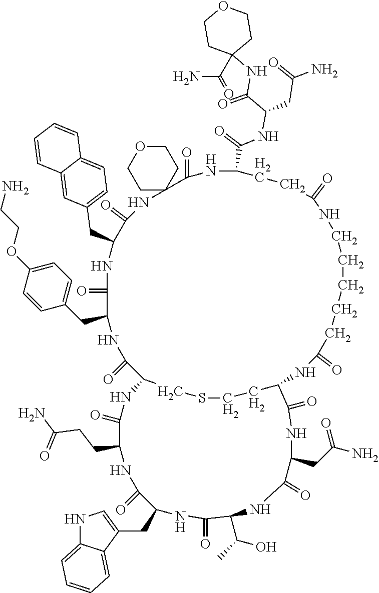 [SMILES: C[C@@H](O)[C@@H]1NC(=O)[C@H](CC(N)=O)NC(=O)[C@@H]2CCSC[C@H](NC(=O)[C@H](CCC(N)=O)NC(=O)[C@H](Cc3c[nH]c4ccccc34)NC1=O)C(=O)N[C@@H](Cc1ccc(OCCN)cc1)C(=O)N[C@@H](Cc1ccc3ccccc3c1)C(=O)NC1(CCOCC1)C(=O)N[C@H](C(=O)N[C@@H](CC(N)=O)C(=O)NC1(C(N)=O)CCOCC1)CCC(=O)NCCCCCC(=O)N2] | |
| 82 | 65 | Ac-[Pen]-E-T-W-[Lys(Ac)]-[Pen]-[Phe(4-(2- | —NH of Phe[4-(2- |
| | | aminoethoxy))]-[2Nal]-[THP]-[Lys(Ac)]-N-[(D)Leu]-NH2 | aminoethoxy)] |
| | | | and C(O) of Glu |
| | | 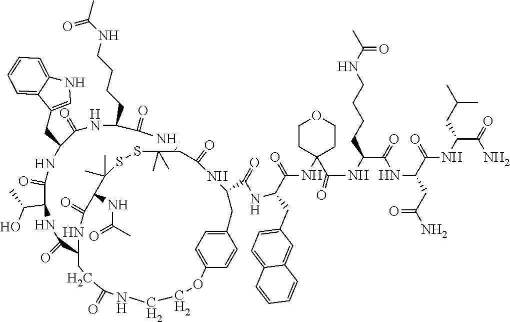 [SMILES: CC(=O)NCCCC[C@@H]1NC(=O)[C@H](Cc2c[nH]c3ccccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@H](CC(C)C)C(N)=O)CCOCC3)NC(=O)[C@@H](NC1=O)C(C)(C)SSC(C)(C)[C@H](NC(C)=O)C(=O)N2] | form a lactam |
| 83 | 66 | Ac-[Pen]-E-T-[W(7-Me)]-[Lys(Ac)]-[Pen]-[Phe(4-(2- aminoethoxy))]-[2Nal]-[THP]-[Lys(Ac)]-N—NH2 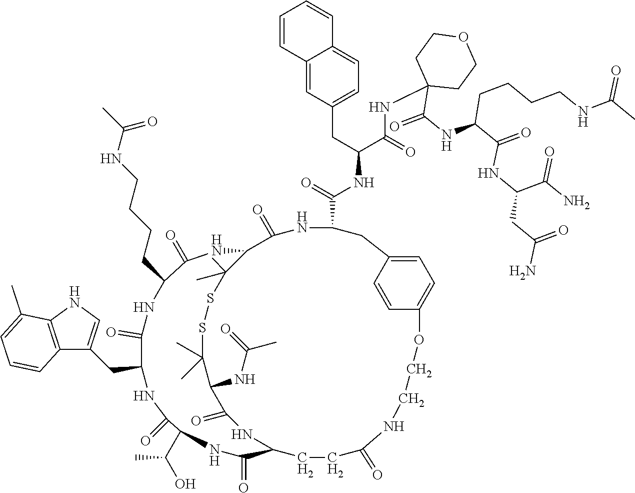 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(N)=O)CCOCC3)NC(=O)[C@@H](NC1=O)C(C)(C)SSC(C)(C)[C@H](NC(C)=O)C(=O)N2] | —NH of Phe[4- (2-aminoethoxy)] and C(O) of Glu form a lactam |
| 84 | 67 | Ac-[Abu]-E-T-W-Q-C-[Phe(4-(2-aminoethoxy))]-[2Nal]- [THP]-[Lys(Ac)]-N-[(D)Leu]-NH2 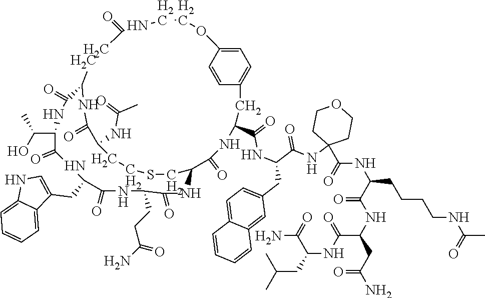 [SMILES: CC(=O)NCCCC[C@H](NC(=O)C1(NC(=O)[C@H](Cc2ccc3ccccc3c2)NC(=O)[C@H](Cc2ccc(OCCNC(C)=O)cc2)NC(=O)[C@@H]2CSCC[C@H](NC(C)=O)C(=O)N[C@@H](C)C(=O)N[C@@H]([C@@H](C)O)C(=O)N[C@@H](Cc3c[nH]c4ccccc34)C(=O)N[C@H](CCC(N)=O)C(=O)N2)CCOCC1)C(=O)N[C@@H](CC(N)=O)C(=O)N[C@H](CC(C)C)C(N)=O] | —NH of Phe[4- (2-aminoethoxy)] and C(O) of Glu form a lactam |
| 85 | 68 | Ac-[Abu]-E-T-[W(7-Me)]-Q-C-[Phe(4-(2-aminoethoxy))]- | —NH of Phe[4-(2- |
| | | [2Nal]-[THP]-[Lys(Ac)]-N—NH2 | aminoethoxy)] |
| | | | and C(O) of Glu |
| | | 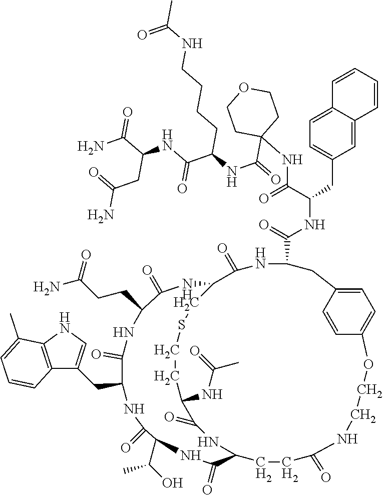 [SMILES: CC(=O)NCCCC[C@@H](NC(=O)C1(NC(=O)[C@H](Cc2ccc3ccccc3c2)NC(=O)[C@@H]2Cc3ccc(cc3)OCCNC(=O)CC[C@@H]3NC(=O)[C@@H](NC(C)=O)CCSC[C@H](NC(=O)[C@H](CCC(N)=O)NC(=O)[C@H](Cc4c[nH]c5c(C)cccc45)NC(=O)[C@H]([C@@H](C)O)NC3=O)C(=O)N2)CCOCC1)C(=O)N[C@@H](CC(N)=O)C(N)=O] | form a lactam |
| 86 | 69 | Ac-[Abu]-E-T-[W(7-Me)]-Q-C-[Phe(4-(2-aminoethoxy))]- | —NH of Phe[4-(2- |
| | | [2Nal]-[THP]-[Lys(Ac)]-NH2 | aminoethoxy)] |
| | | | and C(O) of Glu |
| | | 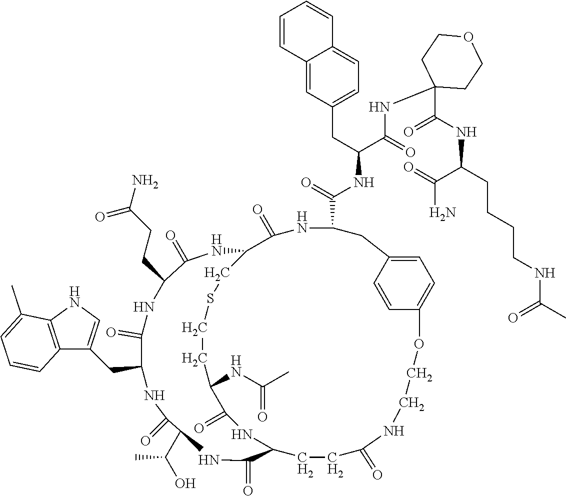 [SMILES: CC(=O)NCCCC[C@H](NC(=O)C1(NC(=O)[C@H](Cc2ccc3ccccc3c2)NC(=O)[C@@H]2Cc3ccc(cc3)OCCNC(=O)CC[C@@H]3NC(=O)[C@@H](NC(C)=O)CCSC[C@H](NC(=O)[C@H](CCC(N)=O)NC(=O)[C@H](Cc4c[nH]c5c(C)cccc45)NC(=O)[C@H]([C@@H](C)O)NC3=O)C(=O)N2)CCOCC1)C(N)=O] | form a lactam |
| 87 | 70 | Ac-[Abu]-E-T-[W(7-Me)]-Q-C-[Phe(4-(2-aminoethoxy))]- | —NH of Phe[4-(2- |
| | | [2Nal]-[THP]-NH2 | aminoethoxy)] |
| | | | and C(O) of Glu |
| | | 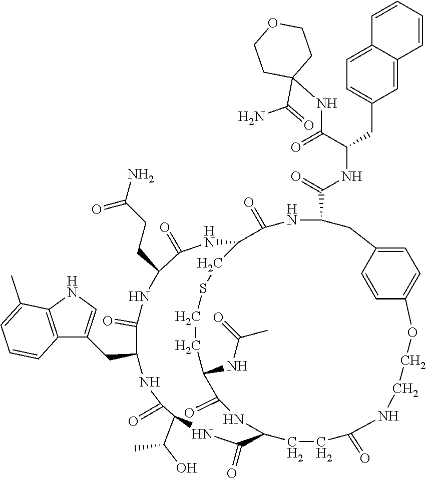 [SMILES: CC(=O)N[C@H]1CCSC[C@@H]2NC(=O)[C@H](CCC(N)=O)NC(=O)[C@H](Cc3c[nH]c4c(C)cccc34)NC(=O)[C@H]([C@@H](C)O)NC(=O)[C@H](CCC(=O)NCCOc3ccc(cc3)C[C@@H](C(=O)N[C@@H](Cc3ccc4ccccc4c3)C(=O)NC3(C(N)=O)CCOCC3)NC2=O)NC1=O] | form a lactam |
| 88 | 71 | Ac-[Abu]-Q-T-W-Q-C-[Phe(4-(2-aminoethoxy))]-[2Nal]- | #N/A |
| | | [THP]-E-N-[bAla]-NH2 | |
| | | 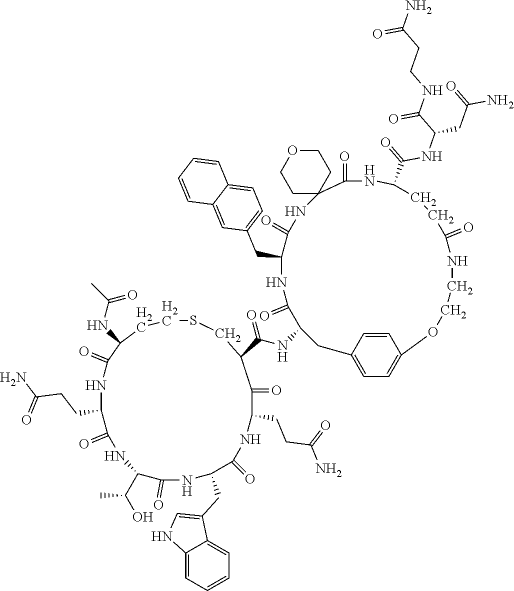 [SMILES: CC(=O)N[C@H]1CCSC[C@@H](C(=O)N[C@H]2Cc3ccc(cc3)OCCNC(=O)CC[C@@H](C(=O)N[C@@H](CC(N)=O)C(=O)NCCC(N)=O)NC(=O)C3(CCOCC3)NC(=O)[C@H](Cc3ccc4ccccc4c3)NC2=O)C(=O)[C@H](CCC(N)=O)NC(=O)[C@H](Cc2c[nH]c3ccccc23)NC(=O)[C@H]([C@@H](C)O)NC(=O)[C@H](CCC(N)=O)NC1=O] | |
| 89 | 72 | Ac-[Abu]-Q-T-W-Q-C-[Phe(4-(2-aminoethoxy))]-[2Nal]- | #N/A |
| | | [THP]-[aMeGlu]-N-[bAla]-NH2 | |
| | | 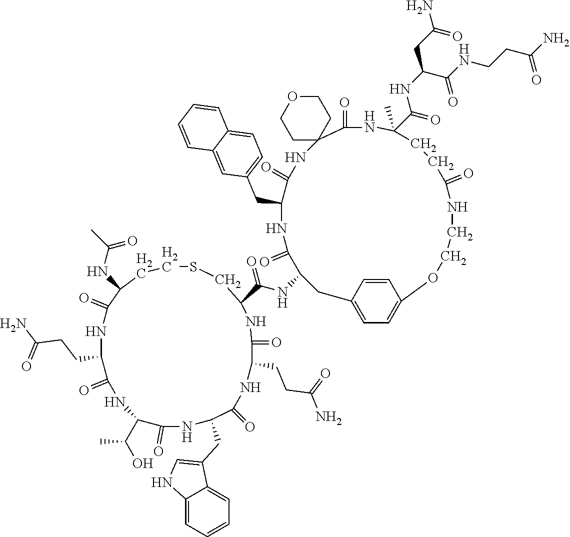 [SMILES: CC(=O)N[C@H]1CCSC[C@@H](C(=O)N[C@H]2Cc3ccc(cc3)OCCNC(=O)CC[C@](C)(C(=O)N[C@@H](CC(N)=O)C(=O)NCCC(N)=O)NC(=O)C3(CCOCC3)NC(=O)[C@H](Cc3ccc4ccccc4c3)NC2=O)NC(=O)[C@H](CCC(N)=O)NC(=O)[C@H](Cc2c[nH]c3ccccc23)NC(=O)[C@H]([C@@H](C)O)NC(=O)[C@H](CCC(N)=O)NC1=O] | |
| 90 | 73 | Ac-[Abu]-Q-T-W-Q-C-[Phe(4-(2-aminoethoxy))]-[2Nal]- | #N/A |
| | | [THP]-[Aad]-N-[bAla]-NH2 | |
| | | 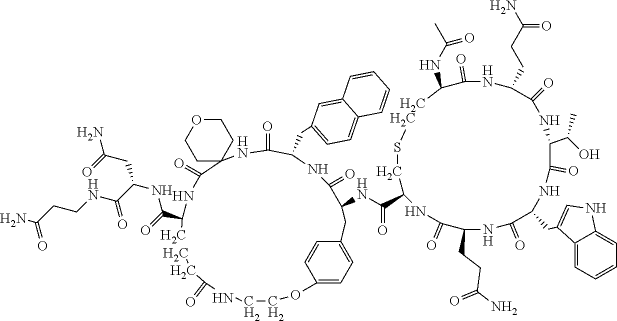 [SMILES: CSC[C@@H](NC(=O)[C@H](CCC(N)=O)NC(=O)[C@@H](Cc1c[nH]c2ccccc12)NC(=O)[C@H](NC(=O)[C@@H](CCC(N)=O)NC(=O)[C@@H](C)NC(C)=O)[C@H](C)O)C(=O)N[C@H]1Cc2ccc(cc2)OCCNC(=O)CCC[C@@H](C(=O)N[C@@H](CC(N)=O)C(=O)NCCC(N)=O)NC(=O)C2(CCOCC2)NC(=O)[C@H](Cc2ccc3ccccc3c2)NC1=O] | |

wherein Pen-Pen form a disulfide bond, or Abu-C form a thioether bond.

TABLE 1E
Compounds

| SEQ | | |
|---|---|---|
| ID. | Cmpd. | Sequence/Structure |
| 91 | 74 | Ac-[(D)Arg]-[Abu]-Q-T-W-Q-C-[Phe(4-(2-aminoethoxy))]-[2Nal]- |
| | | [THP]-E-N-[bAla]-NH2 |
| | | 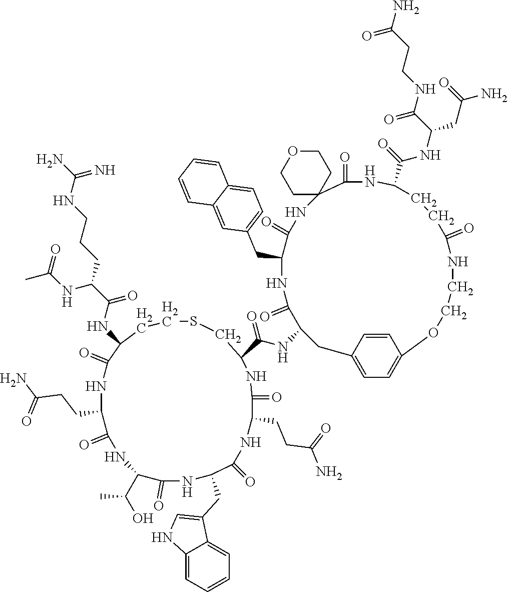 [SMILES: CC(=O)N[C@H](CCCNC(=N)N)C(=O)N[C@H]1CCSC[C@@H](C(=O)N[C@H]2Cc3ccc(cc3)OCCNC(=O)CC[C@@H](C(=O)N[C@@H](CC(N)=O)C(=O)NCCC(N)=O)NC(=O)C3(CCOCC3)NC(=O)[C@H](Cc3ccc4ccccc4c3)NC2=O)NC(=O)[C@H](CCC(N)=O)NC(=O)[C@H](Cc2c[nH]c3ccccc23)NC(=O)[C@H]([C@@H](C)O)NC(=O)[C@H](CCC(N)=O)NC1=O] |
| 92 | 75 | Ac-[Pen]-Q-T-W-Q-[Pen]-[Phe(4-(2-aminoethoxy))]-[2Nal]- |
| | | [THP]-E-N-[bAla]-NH2 |
| | | 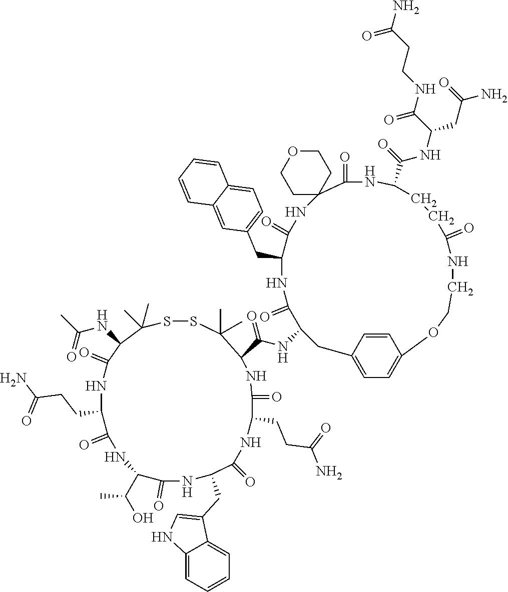 [SMILES: CC(=O)N[C@@H]1C(=O)N[C@@H](CCC(N)=O)C(=O)N[C@@H]([C@@H](C)O)C(=O)N[C@@H](Cc2c[nH]c3ccccc23)C(=O)N[C@@H](CCC(N)=O)C(=O)N[C@H](C(=O)N[C@H]2Cc3ccc(cc3)OCCNC(=O)CC[C@@H](C(=O)N[C@@H](CC(N)=O)C(=O)NCCC(N)=O)NC(=O)C3(CCOCC3)NC(=O)[C@H](Cc3ccc4ccccc4c3)NC2=O)C(C)(C)SSC1(C)C] |
| 93 | 76 | Ac-E-A-E-T-[W(7-Me)]-[Lys(Ac)]-A-[Phe(4-(2-aminoethoxy))]- |
| | | [2Nal]-[THP]-[Lys(Ac)]-N-[(D)Lys]-NH2 |
| | | 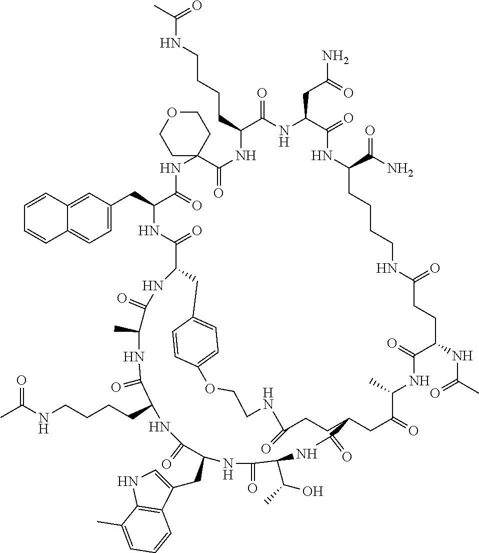 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@H](NC(=O)[C@H](C)NC1=O)C(=O)N[C@@H](Cc1ccc3ccccc3c1)C(=O)NC1(CCOCC1)C(=O)N[C@@H](CCCCNC(C)=O)C(=O)N[C@@H](CC(N)=O)C(=O)N[C@@H](C(N)=O)CCCCNC(=O)CC[C@H](NC(C)=O)C(=O)N[C@@H](C)C(=O)C2] |
| 94 | 77 | [Ac_Morph]-[Pen]-E-T-[W(7-Me)]-[Lys(Ac)]-[Pen]-[Phe(4-(2- |
| | | aminoethoxy))]-[2Nal]-[THP]-[Lys(Ac)]-N-[3Pal]-[Sarc]-NH2 |
| | | 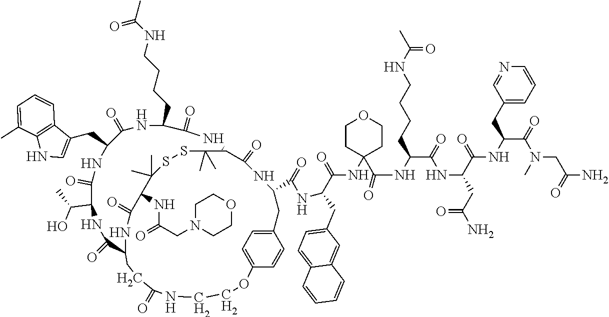 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC(C)(C)[C@H](NC(=O)CN1CCOCC1)C(=O)N2] |
| 95 | 78 | [Ac_Morph]-[Pen]-E-T-[W(7-Me)]-[Lys(Ac)]-[Pen]-[Phe(4-(2- |
| | | aminoethoxy))]-[2Nal]-[aMeLeu]-L-N-[3Pal]-[Sarc]-NH2 |
| | | 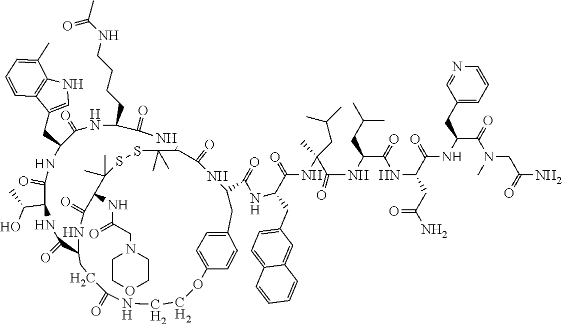 |
| 96 | 79 | [Ac_Morph]-[Pen]-E-T-[W(7-Me)]-[Lys(Ac)]-[Pen]-[Phe(4-(2- |
| | | aminoethoxy))]-[2Nal]-[THP]-[Lys(Ac)]-L-[3Pal]-[Sarc]-NH2 |
| | | 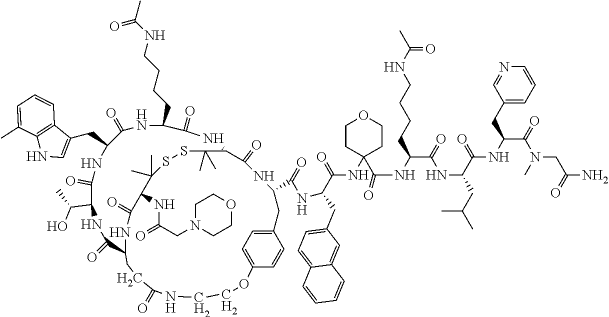 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(C)C)C(=O)N[C@@H](Cc4cccnc4)C(=O)N(C)CC(N)=O)CCOCC3)NC(=O)[C@@H](NC1=O)C(C)(C)SSC(C)(C)[C@H](NC(=O)CN1CCOCC1)C(=O)N2] |
| 97 | 80 | Ac-D-[Pen]-N-T-[W(7-Me)]-[Lys(Ac)]-[Pen]-[Phe(4-CONH2)]- |
| | | [2Nal]-[THP]-[Lys(Ac)]-N-[3Pal]-[(D)Lys]-NH2 |
| | | 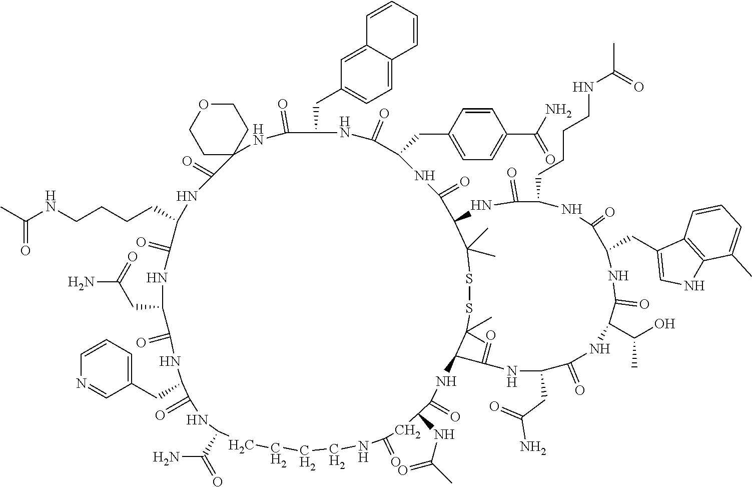 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@@H](NC(C)=O)CC(=O)NCCCC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@@H](Cc3ccc4ccccc4c3)NC(=O)[C@H](Cc3ccc(C(N)=O)cc3)NC(=O)[C@@H](NC1=O)C(C)(C)SSC2(C)C] |
| 98 | 81 | Ac-E-[Pen]-N-T-[W(7-Me)]-[Lys(Ac)]-[Pen]-[Phe(4-CONH2)]- |
| | | [2Nal]-[THP]-[Lys(Ac)]-N-[3Pal]-[(D) Dap]-NH2 |
| | | 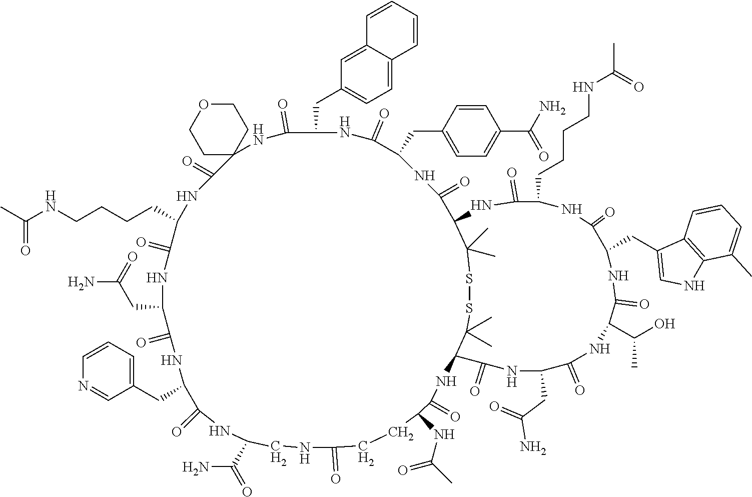 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@@H](NC(C)=O)CCC(=O)NC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@@H](Cc3ccc4ccccc4c3)NC(=O)[C@H](Cc3ccc(C(N)=O)cc3)NC(=O)[C@@H](NC1=O)C(C)(C)SSC2(C)C] |
| 99 | 82 | Ac-D-[Pen]-N-T-[W(7-Me)]-[Lys(Ac)]-[Pen]-[Phe(4-CONH2)]- |
| | | [2Nal]-[THP]-[Lys(Ac)]-N-[3Pal]-[(D)Dap]-NH2 |
| | | 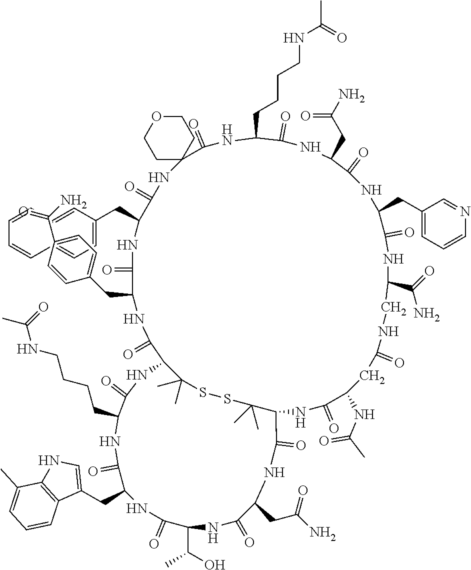 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@@H](NC(C)=O)CC(=O)NC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC(=O)[C@H](Cc3ccc(C(N)=O)cc3)NC(=O)[C@@H](NC1=O)C(C)(C)SSC2(C)C] |
| 100 | 83 | Ac-[Pen]-N-T-[W(7-Me)]-[Lys(Ac)]-[Pen]-[Phe(4-(2- |
| | | aminoethoxy))]-[2Nal]-[THP]-[Lys(Ac)]-N-[3Pal]-[(D)Lys]- |
| | | [NH2]-[PEG2_DiAcid] |
| | | 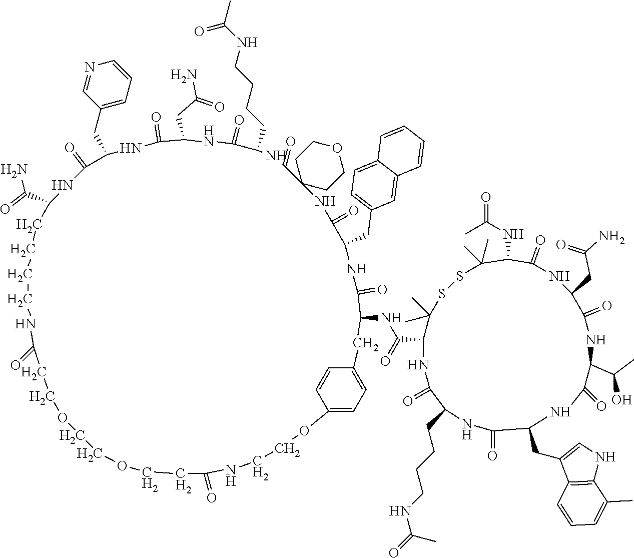 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@H]2Cc3ccc(cc3)OCCNC(=O)CCOCCOCCC(=O)NCCCC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC2=O)NC1=O] |
| 101 | 84 | Ac-[Pen]-N-T-[W(7-Me)]-[Lys(Ac)]-[Pen]-[Phe(4-(2- |
| | | aminoethoxy))]-[2Nal]-[THP]-[Lys(Ac)]-N-[3Pal]-[(D)Asp]-NH2 |
| | | 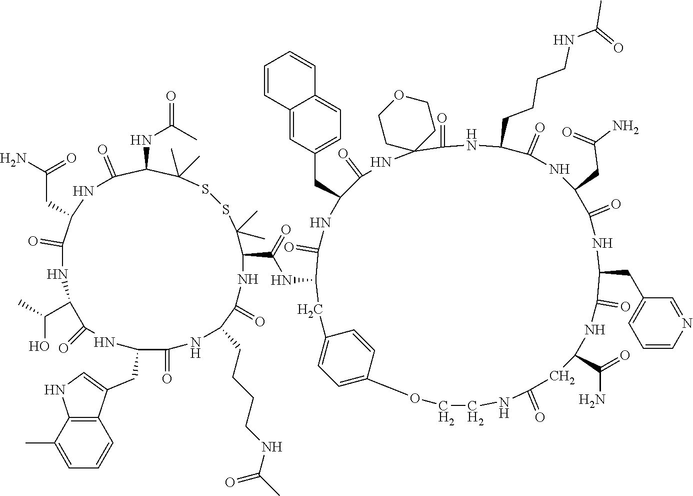 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@H]2Cc3ccc(cc3)OCCNC(=O)C[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC2=O)NC1=O] |
| 102 | 85 | Ac-[Pen]-N-T-[W(7-Me)]-[Lys(Ac)]-[Pen]-[Phe(4-(2- |
| | | aminoethoxy))]-[2Nal]-[THP]-[Lys(Ac)]-N-[3Pal]-[(D)Glu]-NH2 |
| | | 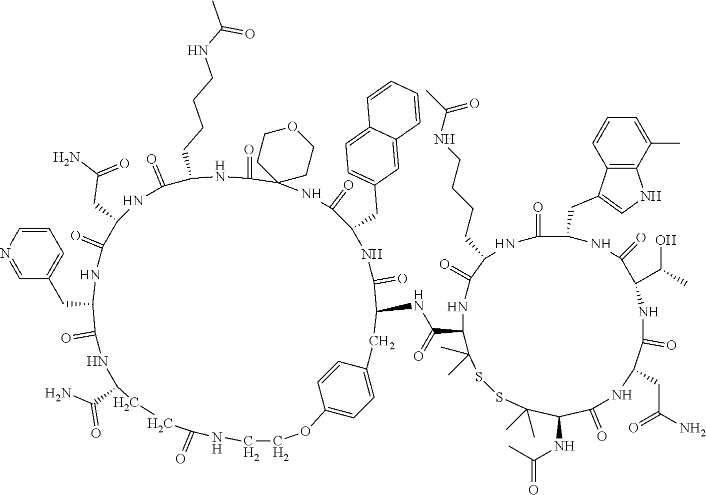 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@H]2Cc3ccc(cc3)OCCNC(=O)CC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC2=O)NC1=O] |
| 103 | 86 | Ac-[Pen]-N-T-[W(7-Me)]-[Lys(Ac)]-[Pen]-[Phe(4-CONH2)]- |
| | | [2Nal]-[THP]-E-N-[3Pal]-[(D)Lys]-NH2 |
| | | 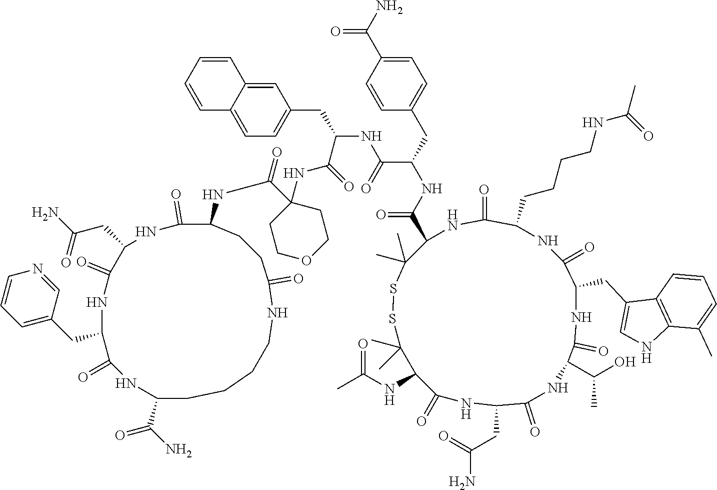 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@H]3CCC(=O)NCCCC[C@H](C(N)=O)NC(=O)[C@H](Cc4cccnc4)NC(=O)[C@H](CC(N)=O)NC3=O)CCOCC2)NC1=O] |
| 104 | 87 | Ac-[Pen]-N-T-[W(7-Me)]-[Lys(Ac)]-[Pen]-[Phe(4-CONH2)]- |
| | | [2Nal]-[THP]-K-N-[3Pal]-[(D)Asp]-NH2 |
| | | 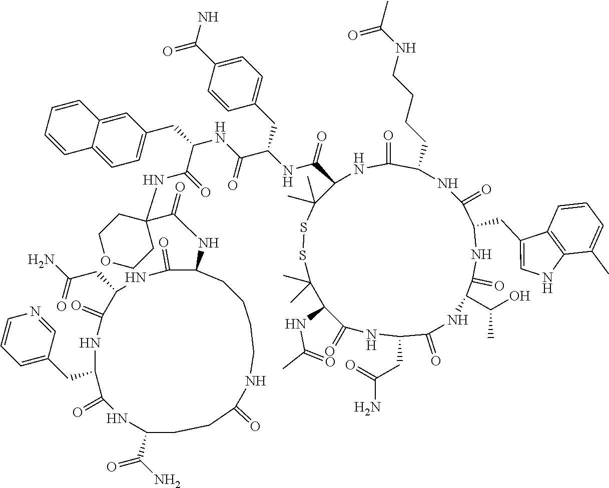 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@H]3CCCCNC(=O)CC[C@H](C(N)=O)NC(=O)[C@H](Cc4cccnc4)NC(=O)[C@H](CC(N)=O)NC3=O)CCOCC2)NC1=O] |
| 105 | 88 | Ac-[Pen]-N-T-[W(7-Me)]-[Lys(Ac)]-[Pen]-[Phe(4-CONH2)]- |
| | | [2Nal]-[THP]-K-N-[3Pal]-[(D)Glu]-NH2 |
| | | 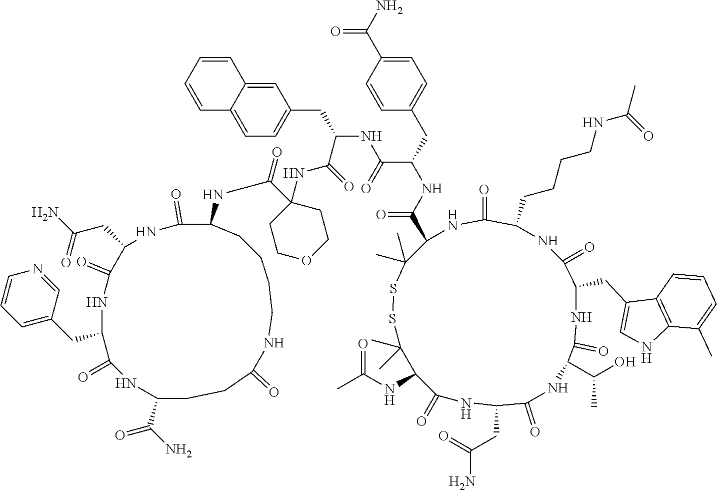 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@H]3CCCCNC(=O)CC[C@H](C(N)=O)NC(=O)[C@H](Cc4cccnc4)NC(=O)[C@H](CC(N)=O)NC3=O)CCOCC2)NC1=O] |
| 106 | 89 | Ac-[Pen]-N-T-[W(7-Me)]-K-[Pen]-[Phe(4-CONH2)]-[2Nal]- |
| | | [THP]-E-N-[3Pal]-[Sarc]-NH2 |
| | | 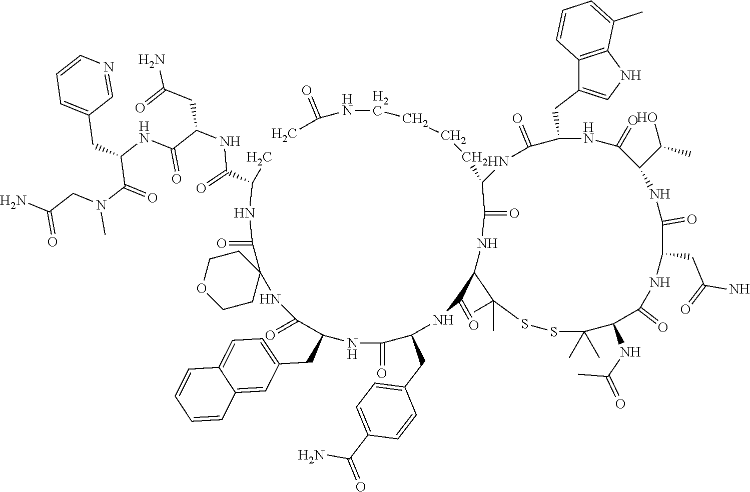 |
| 107 | 90 | Ac-[(D)Lys]-[Pen]-N-T-[W(7-Me)]-[Lys(Ac)]-[Pen]-[Phe(4-(2- |
| | | aminoethoxy))]-[2Nal]-[THP]-[Lys(Ac)]-N-[3Pal]-[(D)Lys]- |
| | | [NH2]-[PEG2_DiAcid] |
| | | 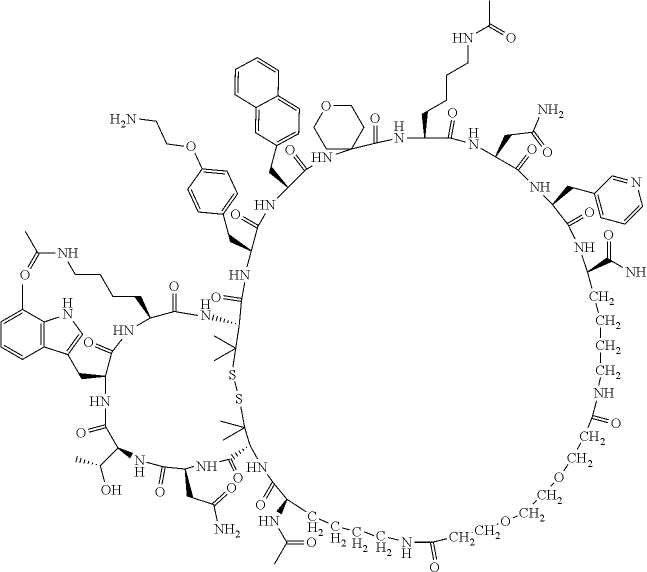 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@H](NC(C)=O)CCCCNC(=O)CCOCCOCCC(=O)NCCCC[C@H](C(N)=O)NC(=O)[C@H](Cc3cccnc3)NC(=O)[C@@H](CC(N)=O)NC(=O)[C@@H](CCCCNC(C)=O)NC(=O)C3(CCOCC3)NC(=O)[C@@H](Cc3ccc4ccccc4c3)NC(=O)[C@@H](Cc3ccc(OCCN)cc3)NC(=O)[C@H](NC1=O)C(C)(C)SSC2(C)C] |
| 108 | 91 | Ac-[Pen]-N-T-[W(7-Me)]-E-[Pen]-[Phe(4-CONH2)]-[2Nal]- |
| | | [THP]-K-N-[3Pal]-[Sarc]-NH2 |
| | | 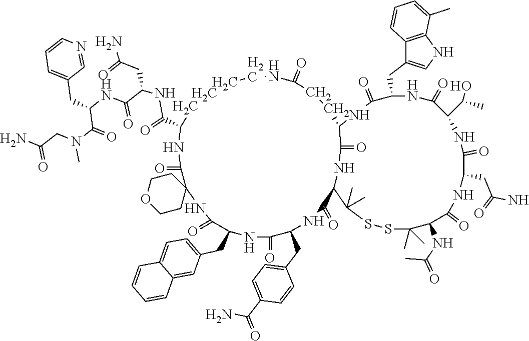 |
| 109 | 92 | Ac-[Pen]-N-T-[W(7-Me)]-[Lys(Ac)]-[Pen]-[Phe(4-CONH2)]- |
| | | [2Nal]-[THP]-[(D)Lys]-N-[3Pal] |
| | | 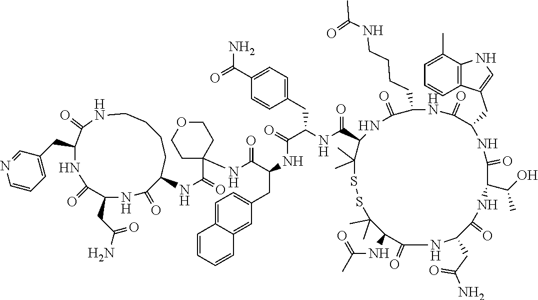 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@@H]3CCCCNC(=O)[C@H](Cc4cccnc4)NC(=O)[C@H](CC(N)=O)NC3=O)CCOCC2)NC1=O] |
| 110 | 93 | Ac-[Pen]-N-T-[W(7-Me)]-[Lys(Ac)]-[Pen]-[Phe(4-CONH2)]- |
| | | [2Nal]-[THP]-[(D)Dap]-N-[3Pal] |
| | | 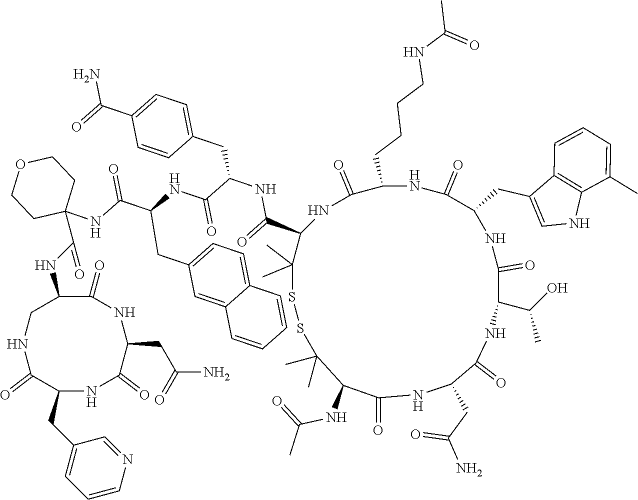 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@@H]3CNC(=O)[C@H](Cc4cccnc4)NC(=O)[C@H](CC(N)=O)NC3=O)CCOCC2)NC1=O] |
| 111 | 94 | Ac-[Pen]-N-T-[W(7-Me)]-[Lys(Ac)]-[Pen]-[Phe(4-CONH2)]- |
| | | [2Nal]-[THP]-[(D)Lys]-N-[3Pal]-[Aib] |
| | | 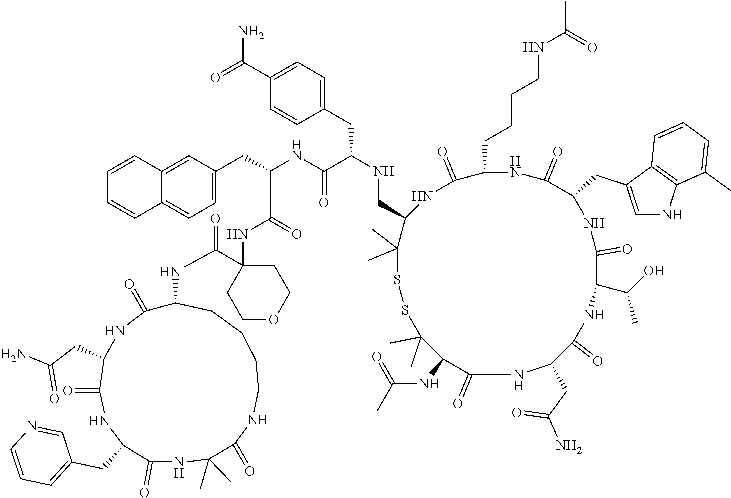 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](CN[C@@H](Cc2ccc(C(N)=O)cc2)C(=O)N[C@@H](Cc2ccc3ccccc3c2)C(=O)NC2(C(=O)N[C@@H]3CCCCNC(=O)C(C)(C)NC(=O)[C@H](Cc4cccnc4)NC(=O)[C@H](CC(N)=O)NC3=O)CCOCC2)NC1=O] |
| 112 | 95 | Ac-[Pen]-N-T-[W(7-Me)]-[Lys(Ac)]-[Pen]-[Phe(4-CONH2)]- |
| | | [2Nal]-[THP]-[(D)Dap]-N-[3Pal]-[Aib] |
| | | 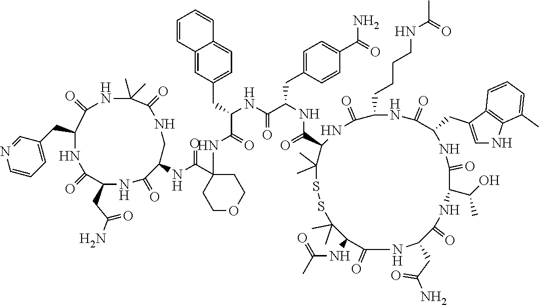 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@@H]3CNC(=O)C(C)(C)NC(=O)[C@H](Cc4cccnc4)NC(=O)[C@H](CC(N)=O)NC3=O)CCOCC2)NC1=O] |
| 113 | 96 | Ac-[Pen]-N-T-[W(7-Me)]-[Lys(Ac)]-[Pen]-[Phe(4-CONH2)]- |
| | | [2Nal]-[THP]-[(D)Lys]-N-[3Pal]-G |
| | | 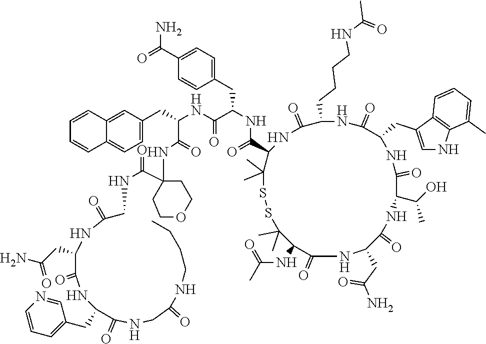 |
| 114 | 97 | Ac-[Pen]-N-T-[W(7-Me)]-[Lys(Ac)]-[Pen]-[Phe(4-CONH2)]- |
| | | [2Nal]-[THP]-[(D)Dap]-N-[3Pal]-G |
| | | 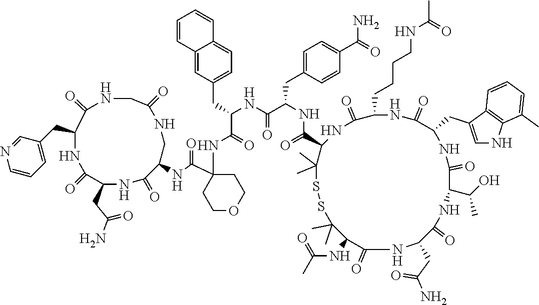 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@@H]3CNC(=O)CNC(=O)[C@H](Cc4cccnc4)NC(=O)[C@H](CC(N)=O)NC3=O)CCOCC2)NC1=O] |

wherein Pen-Pen form a disulfide bond, or Abu-C form a thioether bond.

TABLE 1F
Compounds

| | Cmpd. | Sequence/Structure | Note |
|---|---|---|---|
| 115 | 98 | Ac-[Pen]-N-T-[W(7-Me)]-[Lys(Ac)]-[Pen]-[Phe(4-CONH2)]-[2Nal]- | #N/A |
| | | [THP]-[(D)Lys]-N-[3Pal]-[bAla] | |
| | | 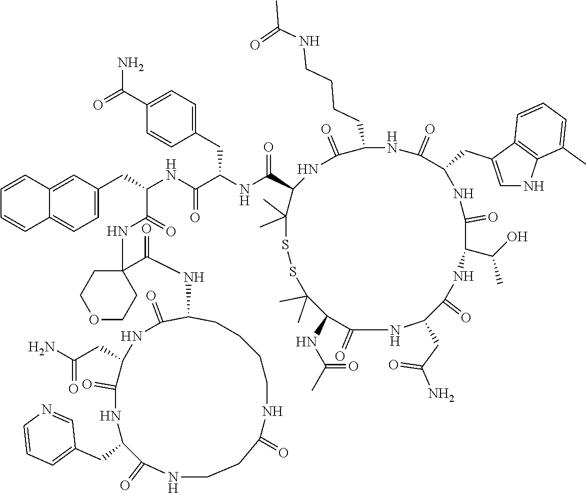 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@@H]3CCCCNC(=O)CCNC(=O)[C@H](Cc4cccnc4)NC(=O)[C@H](CC(N)=O)NC3=O)CCOCC2)NC1=O] | |
| 116 | 99 | Ac-[Pen]-N-T-[W(7-Me)]-[Lys(Ac)]-[Pen]-[Phe(4-CONH2)]-[2Nal]- | #N/A |
| | | [THP]-[(D)Dap]-N-[3Pal]-[bAla] | |
| | | 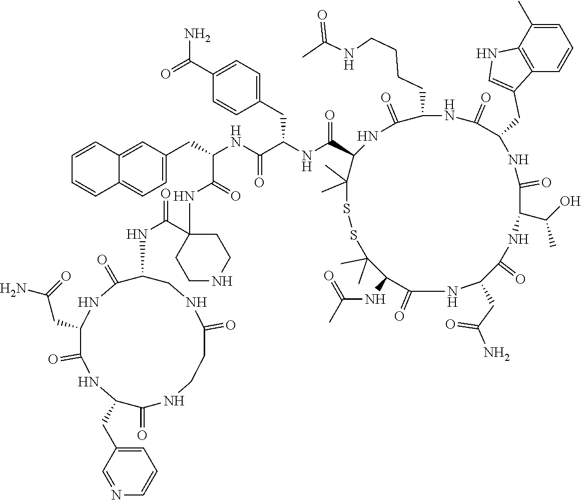 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@@H]3CNC(=O)CCNC(=O)[C@H](Cc4cccnc4)NC(=O)[C@H](CC(N)=O)NC3=O)CCNCC2)NC1=O] | |
| 117 | 100 | Ac-[Pen]-L-T-[W(7-Me)]-[Lys(Ac)]-[Pen]-[Phe(4-OMe)]-[2Nal]- | #N/A |
| | | [THP]-[(D)Lys]-L-[3Pal]-[Sar] | |
| | | 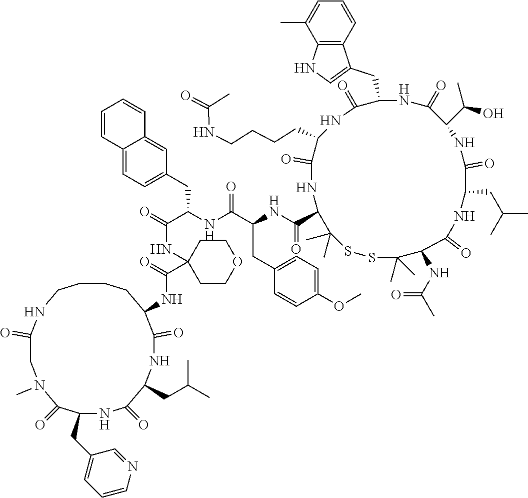 [SMILES: COc1ccc(C[C@H](NC(=O)[C@H]2NC(=O)[C@H](CCCCNC(C)=O)NC(=O)[C@H](Cc3c[nH]c4c(C)cccc34)NC(=O)[C@H]([C@@H](C)O)NC(=O)[C@H](CC(C)C)NC(=O)[C@@H](NC(C)=O)C(C)(C)SSC2(C)C)C(=O)N[C@@H](Cc2ccc3ccccc3c2)C(=O)NC2(C(=O)N[C@@H]3CCCCNC(=O)CN(C)C(=O)[C@H](Cc4cccnc4)NC(=O)[C@H](CC(C)C)NC3=O)CCOCC2)cc1] | |
| 118 | 101 | Ac-E-[Pen]-N-T-[W(7-Me)]-[Lys(Ac)]-[Pen]-[Phe(4-(2- | #N/A |
| | | aminoethoxy))]-[2Nal]-[THP]-[Lys(Ac)]-N-[3Pal]-[(D)Lys]-NH2 | |
| | | 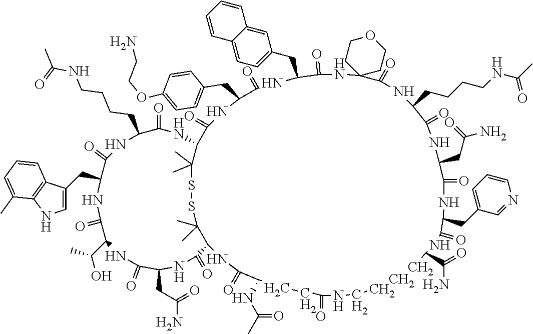 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@@H](NC(C)=O)CCC(=O)NCCCC[C@@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C] | |
| 119 | 102 | Ac-[Pen]-L-T-[W(7-Me)]-[Lys(Ac)]-[Pen]-[Phe(4-CONH2)]-[2Nal]- | #N/A |
| | | [THP]-[(D)Lys]-N-[3Pal]-[Sar] | |
| | | 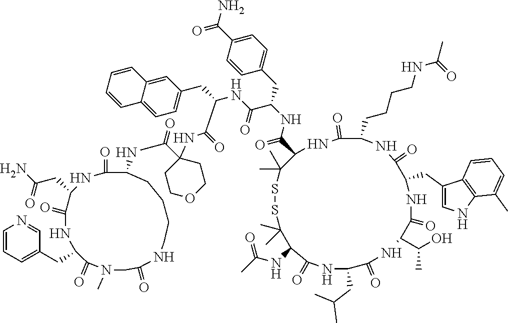 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(C)C)NC(=O)[C@@H](NC(C)=O)C(C)(C)SSC(C)(C)[C@@H](C(=O)N[C@@H](Cc2ccc(C(N)=O)cc2)C(=O)N[C@@H](Cc2ccc3ccccc3c2)C(=O)NC2(C(=O)N[C@@H]3CCCCNC(=O)CN(C)C(=O)[C@H](Cc4cccnc4)NC(=O)[C@H](CC(N)=O)NC3=O)CCOCC2)NC1=O] | |
| 120 | 103 | Ac-[(D)Lys]-[Pen]-N-T-[W(7-Me)]-[Lys(Ac)]-[Pen]-[Phe(4- | #N/A |
| | | CONH2)]-[2Nal]-[THP]-[Lys(Ac)]-N-[3Pal]-[(D)Lys]-[NH2]- | |
| | | [PEG2DA] | |
| | | 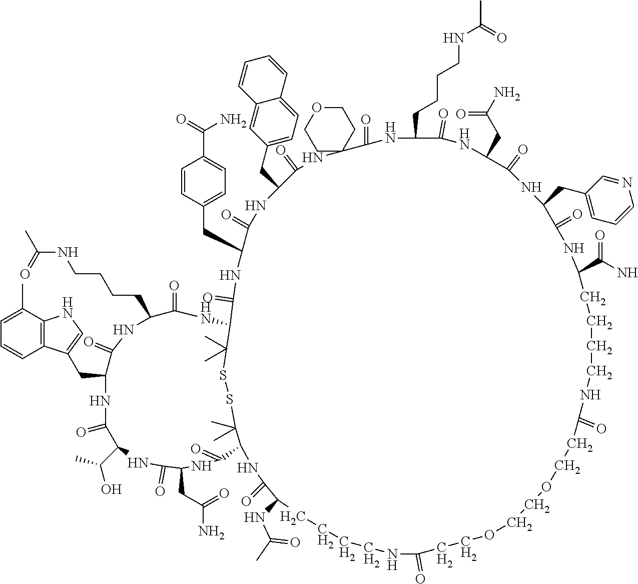 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@H](NC(C)=O)CCCCNC(=O)CCOCCOCCC(=O)NCCCC[C@H](C(N)=O)NC(=O)[C@H](Cc3cccnc3)NC(=O)[C@@H](CC(N)=O)NC(=O)[C@@H](CCCCNC(C)=O)NC(=O)C3(CCOCC3)NC(=O)[C@@H](Cc3ccc4ccccc4c3)NC(=O)[C@@H](Cc3ccc(C(N)=O)cc3)NC(=O)[C@H](NC1=O)C(C)(C)SSC2(C)C] | |
| 121 | 104 | Ac-[(D)Lys]-[Pen]-N-T-[W(7-Me)]-[Lys(Ac)]-[Pen]-[Phe(4- | #N/A |
| | | CONH2)]-[2Nal]-[THP]-[Lys(Ac)]-N-[3Pal]-[(D)Glu]-NH2 | |
| | | 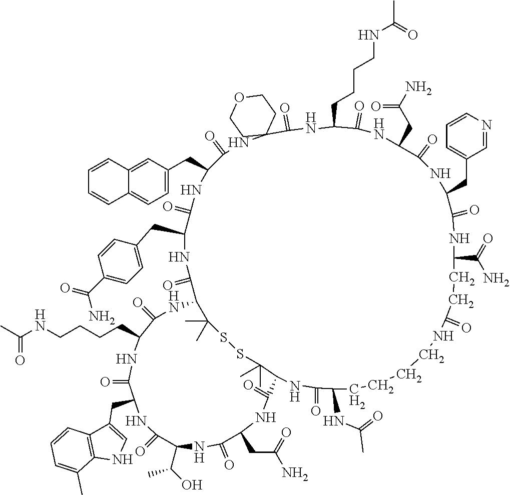 [SMILES: CC(=O)NCCCC[C@@H]1NC(=O)[C@@H](Cc2c[nH]c3c(C)cccc23)NC(=O)[C@H]([C@@H](C)O)NC(=O)[C@H](CC(N)=O)NC(=O)[C@H]2NC(=O)[C@H](NC(C)=O)CCCCNC(=O)CC[C@@H](C(N)=O)NC(=O)[C@H](Cc3cccnc3)NC(=O)[C@@H](CC(N)=O)NC(=O)[C@H](CCCCNC(C)=O)NC(=O)C3(CCOCC3)NC(=O)[C@H](Cc3ccc4ccccc4c3)NC(=O)[C@H](Cc3ccc(C(N)=O)cc3)NC(=O)[C@@H](NC1=O)C(C)(C)SSC2(C)C] | |
| 122 | 105 | Ac-[(D)Lys]-[Pen]-N-T-[W(7-Me)]-[Lys(Ac)]-[Pen]-[Phe(4- | #N/A |
| | | CONH2)]-[2Nal]-[THP]-[Lys(Ac)]-N-[3Pal]-[(D)Asp]-NH2 | |
| | | 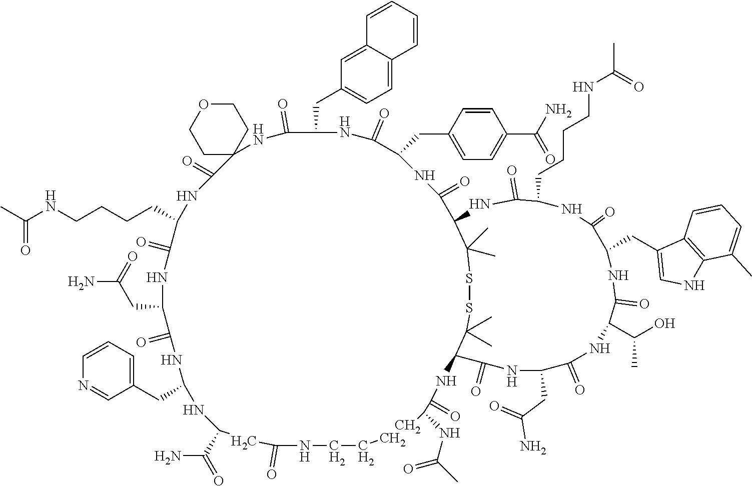 [SMILES: CCCNC(=O)C[C@@H](N[C@H](Cc1cccnc1)NC(=O)[C@H](CC(N)=O)NC(=O)[C@H](CCCCNC(C)=O)NC(=O)C1(NC(=O)[C@@H](Cc2ccc3ccccc3c2)NC(=O)[C@H](Cc2ccc(C(N)=O)cc2)NC(=O)[C@H]2NC(=O)[C@H](CCCCNC(C)=O)NC(=O)[C@H](Cc3c[nH]c4c(C)cccc34)NC(=O)[C@H]([C@@H](C)O)NC(=O)[C@H](CC(N)=O)NC(=O)[C@@H](NC(=O)[C@@H](C)NC(C)=O)C(C)(C)SSC2(C)C)CCOCC1)C(N)=O] | |
| 123 | 106 | Ac-[(D)Dap]-[Pen]-N-T-[W(7-Me)]-[Lys(Ac)]-[Pen]-[Phe(4- | #N/A |
| | | CONH2)]-[2Nal]-[THP]-[Lys(Ac)]-N-[3Pal]-[(D)Glu]-NH2 | |
| | | 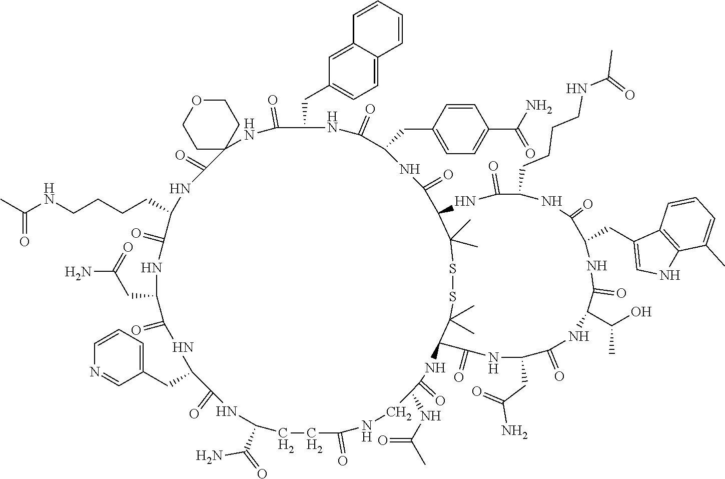 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@H](NC(C)=O)CNC(=O)CC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@@H](Cc3ccc4ccccc4c3)NC(=O)[C@H](Cc3ccc(C(N)=O)cc3)NC(=O)[C@@H](NC1=O)C(C)(C)SSC2(C)C] | |
| 124 | 107 | Ac-[(D)Dap]-[Pen]-N-T-[W(7-Me)]-[Lys(Ac)]-[Pen]-[Phe(4- | #N/A |
| | | CONH2)]-[2Nal]-[THP]-[Lys(Ac)]-N-[3Pal]-[(D) Asp]-NH2 | |
| | | 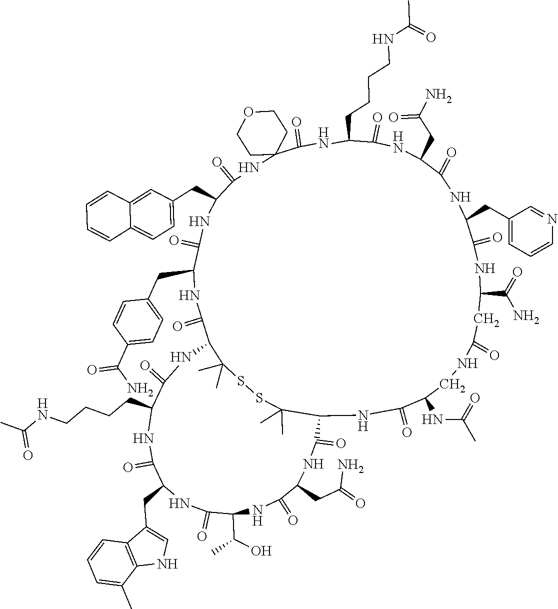 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@H](NC(C)=O)CNC(=O)C[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC(=O)[C@H](Cc3ccc(C(N)=O)cc3)NC(=O)[C@@H](NC1=O)C(C)(C)SSC2(C)C] | |
| 125 | 108 | Ac-E-[Pen]-N-T-[W(7-Me)]-[Lys(Ac)]-[Pen]-[Phe(4-CONH2)]- | #N/A |
| | | [2Nal]-[THP]-[Lys(Ac)]-N-[3Pal]-[(D)Lys]-NH2 | |
| | | 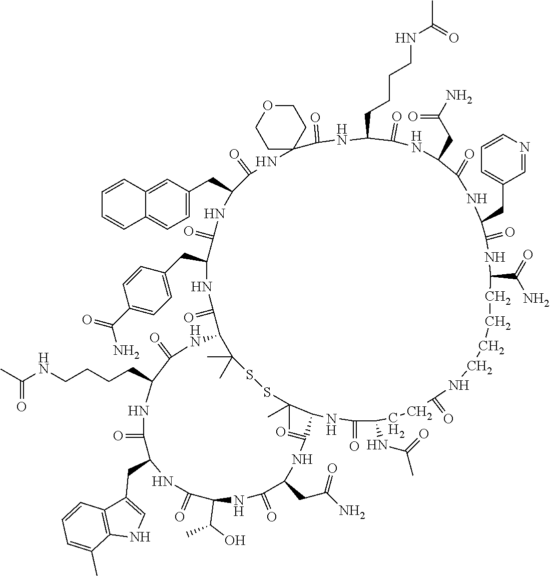 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@@H](NC(C)=O)CCC(=O)NCCCC[C@H](C(N)=O)NC(=O)[C@H](Cc3cccnc3)NC(=O)[C@H](CC(N)=O)NC(=O)[C@H](CCCCNC(C)=O)NC(=O)C3(CCOCC3)NC(=O)[C@H](Cc3ccc4ccccc4c3)NC(=O)[C@H](Cc3ccc(C(N)=O)cc3)NC(=O)[C@@H](NC1=O)C(C)(C)SSC2(C)C] | |
| 126 | 109 | [PEG12_OMe]-[Pen]-E-L-[W(7-Me)]-[Lys(Ac)]-[Pen]-[Phe(4-(2- | #N/A |
| | | aminoethoxy))]-[2Nal]-[aMeLeu]-L-N-[3Pal]-[Sarc]-NH2 | |
| | | 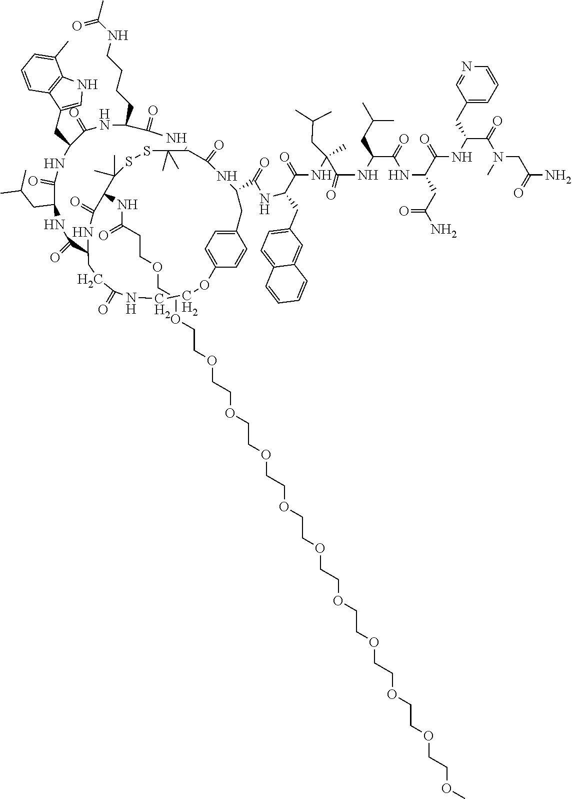 [SMILES: COCCOCCOCCOCCOCCOCCOCCOCCOCCOCCOCCOCCC(=O)N[C@@H]1C(=O)N[C@@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CC(C)C)C(=O)N[C@@H](CC(N)=O)C(=O)N[C@H](Cc3cccnc3)C(=O)N(C)CC(N)=O)NC(=O)[C@@H](NC(=O)[C@H](CCCCNC(C)=O)NC(=O)[C@H](Cc3c[nH]c4c(C)cccc34)NC(=O)[C@H](CC(C)C)NC2=O)C(C)(C)SSC1(C)C] | |
| 127 | 110 | [PEG12_OMe]-[Pen]-E-T-[W(7-Me)]-[Lys(Ac)]-[Pen]-[Phe(4-(2- | #N/A |
| | | aminoethoxy))]-[2Nal]-[aMeLeu]-[Lys(Ac)]-N-[3Pal]-[Sarc]-NH2 | |
| | | 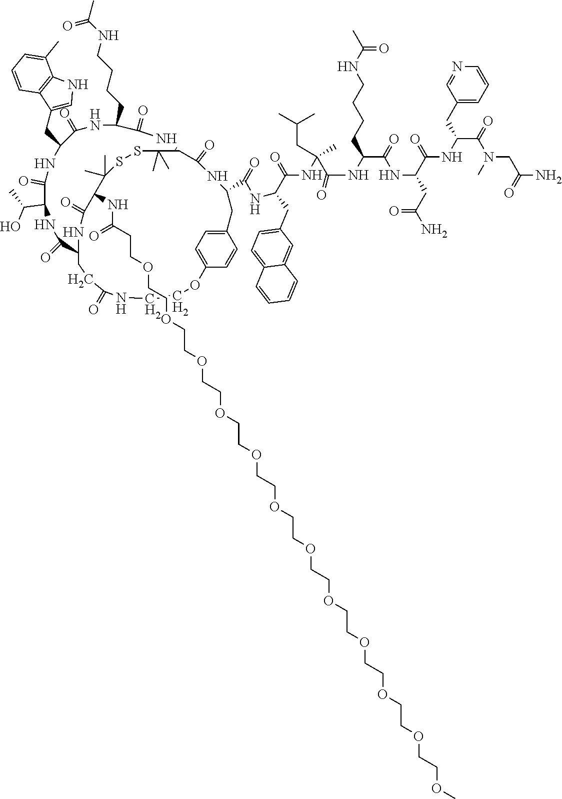 [SMILES: COCCOCCOCCOCCOCCOCCOCCOCCOCCOCCOCCOCCC(=O)N[C@@H]1C(=O)N[C@@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CCCCNC(C)=O)C(=O)N[C@@H](CC(N)=O)C(=O)N[C@H](Cc3cccnc3)C(=O)N(C)CC(N)=O)NC(=O)[C@@H](NC(=O)[C@H](CCCCNC(C)=O)NC(=O)[C@H](Cc3c[nH]c4c(C)cccc34)NC(=O)[C@H]([C@@H](C)O)NC2=O)C(C)(C)SSC1(C)C] | |
| 128 | 111 | Ac-[Pen]-E-T-[W(7-Me)]-[Lys(Ac_Morph)]-[Pen]-[Phe(4-(2- | #N/A |
| | | aminoethoxy))]-[2Nal]-[THP]-[Lys(Ac)]-N-[3Pal]-[Sarc]-NH2 | |
| | | 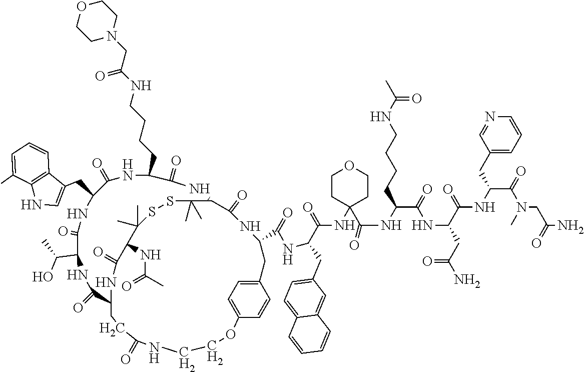 [SMILES: CC(=O)NCCCC[C@H](NC(=O)C1(NC(=O)[C@H](Cc2ccc3ccccc3c2)NC(=O)[C@@H]2Cc3ccc(cc3)OCCNC(=O)CC[C@H]3NC(=O)[C@@H](NC(C)=O)C(C)(C)SSC(C)(C)[C@H](NC(=O)[C@H](CCCCNC(=O)CN4CCOCC4)NC(=O)[C@H](Cc4c[nH]c5c(C)cccc45)NC(=O)[C@H]([C@@H](C)O)NC3=O)C(=O)N2)CCOCC1)C(=O)N[C@@H](CC(N)=O)C(=O)N[C@H](Cc1cccnc1)C(=O)N(C)CC(N)=O] | |
| 129 | 112 | Ac-[Pen]-E-T-[W(7-Me)]-[Lys(PEG12_OMe)]-[Pen]-[Phe(4-(2- | #N/A |
| | | aminoethoxy))]-[2Nal]-[THP]-[Lys(Ac)]-N-[3Pal]-[Sarc]-NH2 | |
| | | 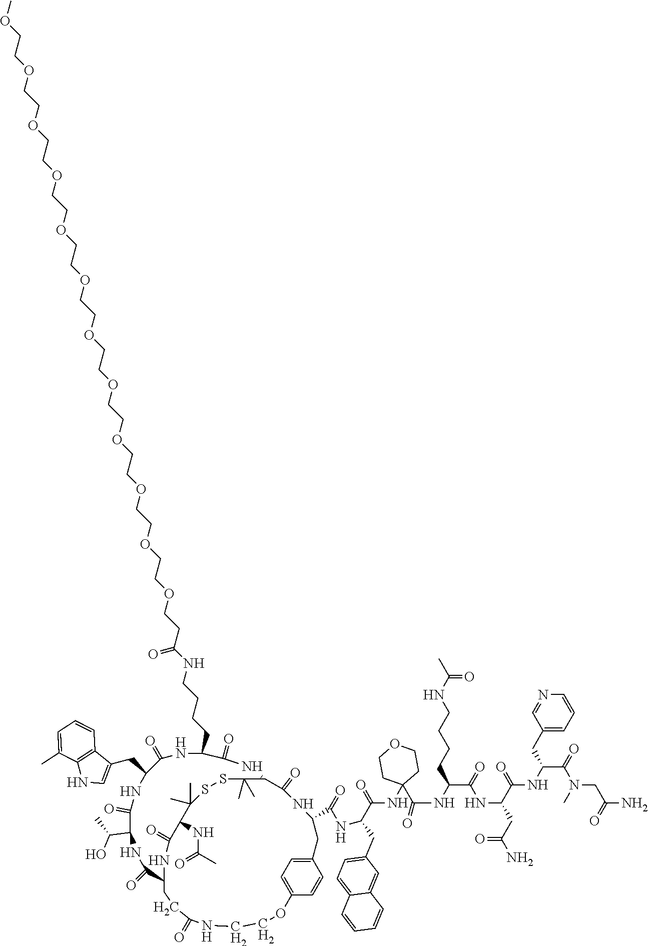 [SMILES: COCCOCCOCCOCCOCCOCCOCCOCCOCCOCCOCCOCCC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | |
| 130 | 113 | Ac-[Pen]-E-L-[W(7-Me)]-[Lys(PEG12_OMe)]-[Pen]-[Phe(4-(2- | #N/A |
| | | aminoethoxy))]-[2Nal]-[aMeLeu]-L-N-[3Pal]-[Sarc]-NH2 | |
| | | 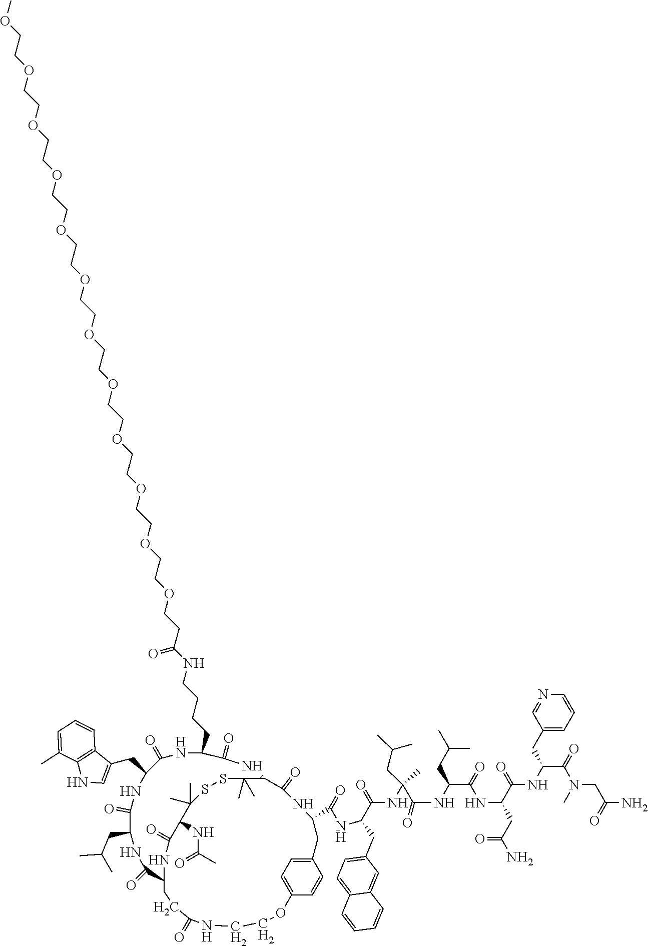 [SMILES: COCCOCCOCCOCCOCCOCCOCCOCCOCCOCCOCCOCCC(=O)NCCCC[C@@H]1NC(=O)[C@H](Cc2c[nH]c3c(C)cccc23)NC(=O)[C@H](CC(C)C)NC(=O)[C@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CC(C)C)C(=O)N[C@@H](CC(N)=O)C(=O)N[C@H](Cc3cccnc3)C(=O)N(C)CC(N)=O)NC(=O)[C@@H](NC1=O)C(C)(C)SSC(C)(C)[C@H](NC(C)=O)C(=O)N2] | |
| 131 | 114 | Ac-[Pen]-E-L-[W(7-Me)]-[Lys(Ac_Morph)]-[Pen]-[Phe(4-(2- | #N/A |
| | | aminoethoxy))]-[2Nal]-[aMeLeu]-L-N-[3Pal]-[Sarc]-NH2 | |
| | | 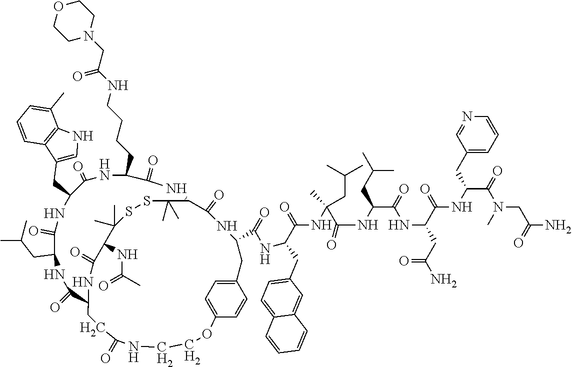 [SMILES: CC(=O)N[C@@H]1C(=O)N[C@@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CC(C)C)C(=O)N[C@@H](CC(N)=O)C(=O)N[C@H](Cc3cccnc3)C(=O)N(C)CC(N)=O)NC(=O)[C@@H](NC(=O)[C@H](CCCCNC(=O)CN3CCOCC3)NC(=O)[C@H](Cc3c[nH]c4c(C)cccc34)NC(=O)[C@H](CC(C)C)NC2=O)C(C)(C)SSC1(C)C] | |
| 132 | 115 | Ac-[Pen]-E-L-[W(7-Me)]-[Lys(Ac_Morph)]-[Pen]-[Phe(4-(2- | #N/A |
| | | aminoethoxy))]-[2Nal]-[THP]-L-N-[3Pal]-[Sarc]-NH2 | |
| | | 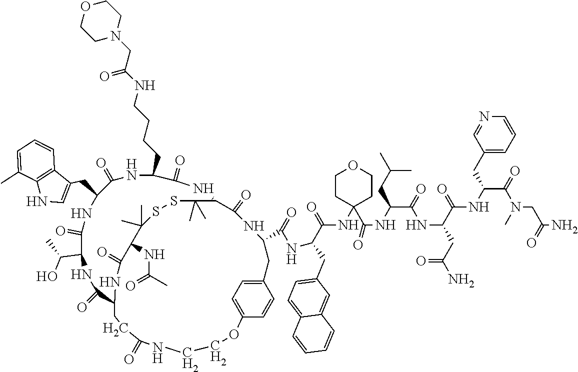 [SMILES: CC(=O)N[C@@H]1C(=O)N[C@@H]2CCC(=O)NCCOc3ccc(cc3)C[C@@H](C(=O)N[C@@H](Cc3ccc4ccccc4c3)C(=O)NC3(C(=O)N[C@@H](CC(C)C)C(=O)N[C@@H](CC(N)=O)C(=O)N[C@H](Cc4cccnc4)C(=O)N(C)CC(N)=O)CCOCC3)NC(=O)[C@@H](NC(=O)[C@H](CCCCNC(=O)CN3CCOCC3)NC(=O)[C@H](Cc3c[nH]c4c(C)cccc34)NC(=O)[C@H]([C@@H](C)O)NC2=O)C(C)(C)SSC1(C)C] | |
| 133 | 116 | Ac-[Pen]-E-L-[W(7-Me)]-[Lys(PEG12_OMe)]-[Pen]-[Phe(4-(2- | #N/A |
| | | aminoethoxy))]-[2Nal]-[THP]-L-N-[3Pal]-[Sarc]-NH2 | |
| | | 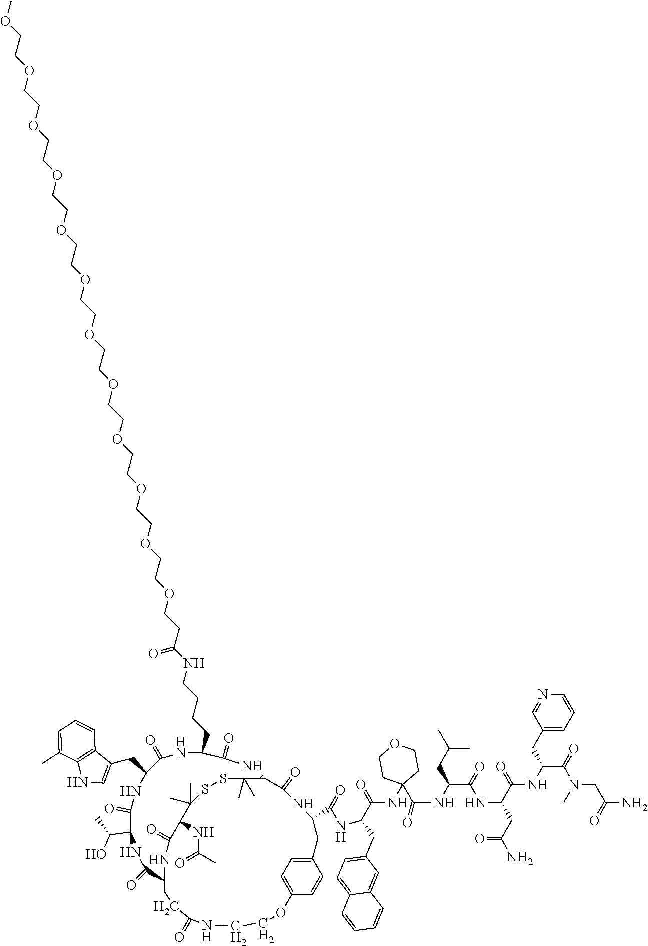 [SMILES: COCCOCCOCCOCCOCCOCCOCCOCCOCCOCCOCCOCCC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CC(C)C)C(=O)N[C@@H](CC(N)=O)C(=O)N[C@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | |
| 134 | 117 | Ac-[Pen]-E-T-[W(7-Me)]-[Lys(PEG12_OMe)]-[Pen]-[Phe(4-(2- | #N/A |
| | | aminoethoxy))]-[2Nal]-[aMeLeu]-L-N-[3Pal]-[Sarc]-NH2 | |
| | | 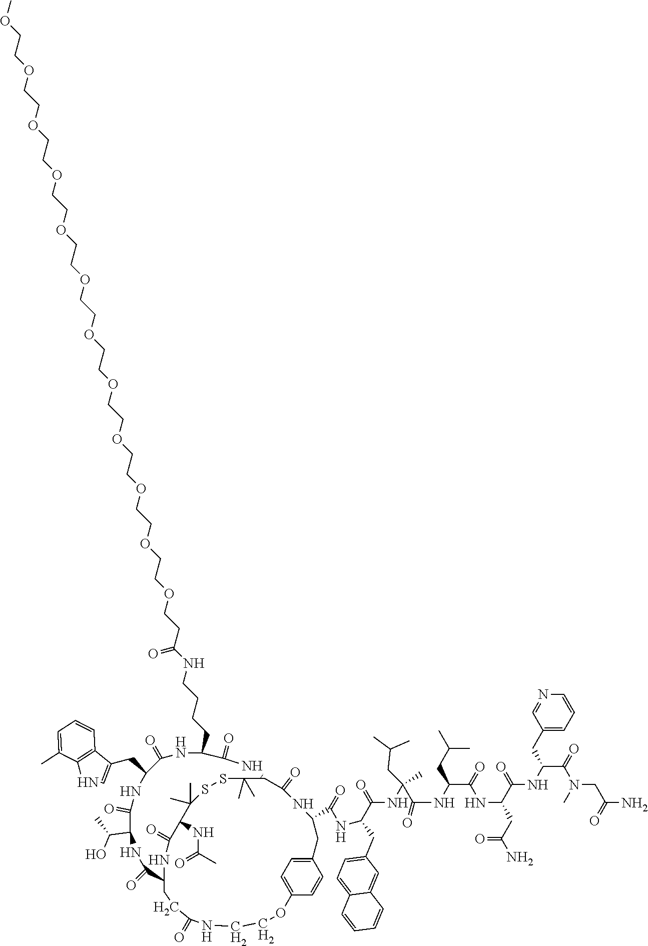 [SMILES: COCCOCCOCCOCCOCCOCCOCCOCCOCCOCCOCCOCCC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CC(C)C)C(=O)N[C@@H](CC(N)=O)C(=O)N[C@H](Cc3cccnc3)C(=O)N(C)CC(N)=O)NC(=O)[C@@H](NC1=O)C(C)(C)SSC(C)(C)[C@H](NC(C)=O)C(=O)N2] | |
| 135 | 118 | Ac-[Pen]-E-T-[W(7-Me)]-[Lys(PEG12_OMe)]-[Pen]-[Phe(4-(2- | #N/A |
| | | aminoethoxy))]-[2Nal]-[aMeLeu]-[Lys(Ac)]-L-[3Pal]-[Sarc]-NH2 | |
| | | 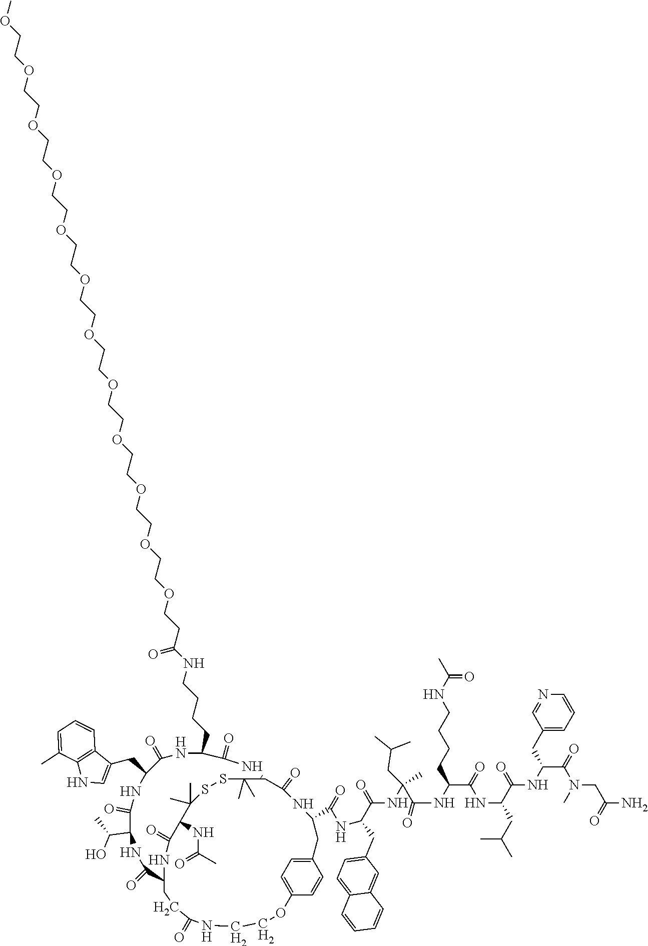 [SMILES: COCCOCCOCCOCCOCCOCCOCCOCCOCCOCCOCCOCCC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CCCCNC(C)=O)C(=O)N[C@@H](CC(C)C)C(=O)N[C@H](Cc3cccnc3)C(=O)N(C)CC(N)=O)NC(=O)[C@@H](NC1=O)C(C)(C)SSC(C)(C)[C@H](NC(C)=O)C(=O)N2] | |
| 136 | 119 | Ac-[Pen]-E-T-[W(7-Me)]-[Lys(PEG12_OMe)]-[Pen]-[Phe(4-(2- | #N/A |
| | | aminoethoxy))]-[2Nal]-[THP]-[Lys(Ac)]-L-[3Pal]-[Sarc]-NH2 | |
| | | 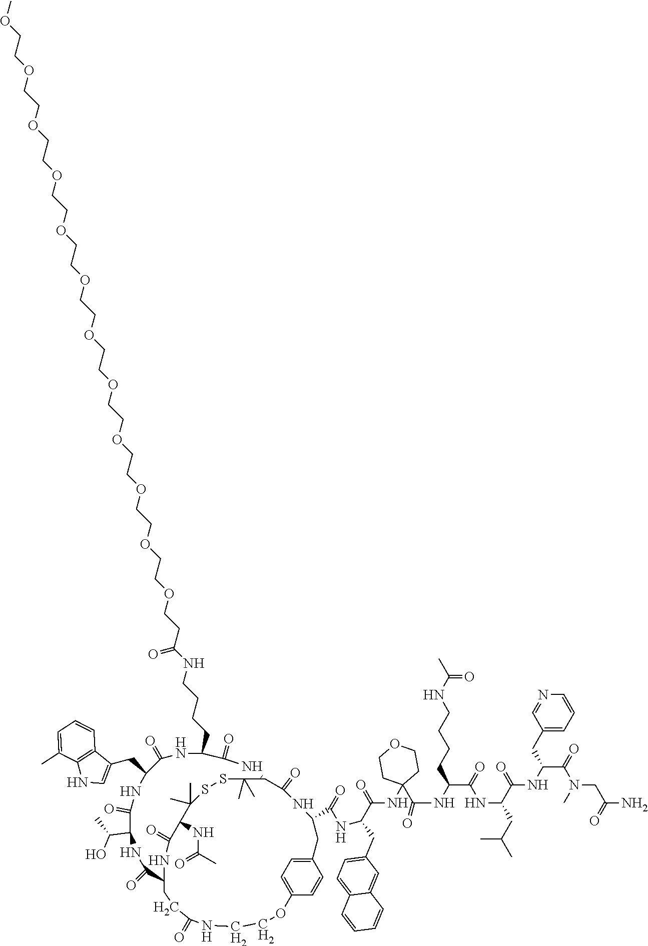 [SMILES: COCCOCCOCCOCCOCCOCCOCCOCCOCCOCCOCCOCCC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(C)C)C(=O)N[C@H](Cc4cccnc4)C(=O)N(C)CC(N)=O)CCOCC3)NC(=O)[C@@H](NC1=O)C(C)(C)SSC(C)(C)[C@H](NC(C)=O)C(=O)N2] | |
| 137 | 120 | Ac-[Pen]-E-T-[W(7-Me)]-[Lys(PEG12_OMe)]-[Pen]-[Phe(4-(2- | #N/A |
| | | aminoethoxy))]-[2Nal]-[aMeLeu]-[Lys(Ac)]-N-[3Pal]-[Sarc]-NH2 | |
| | | 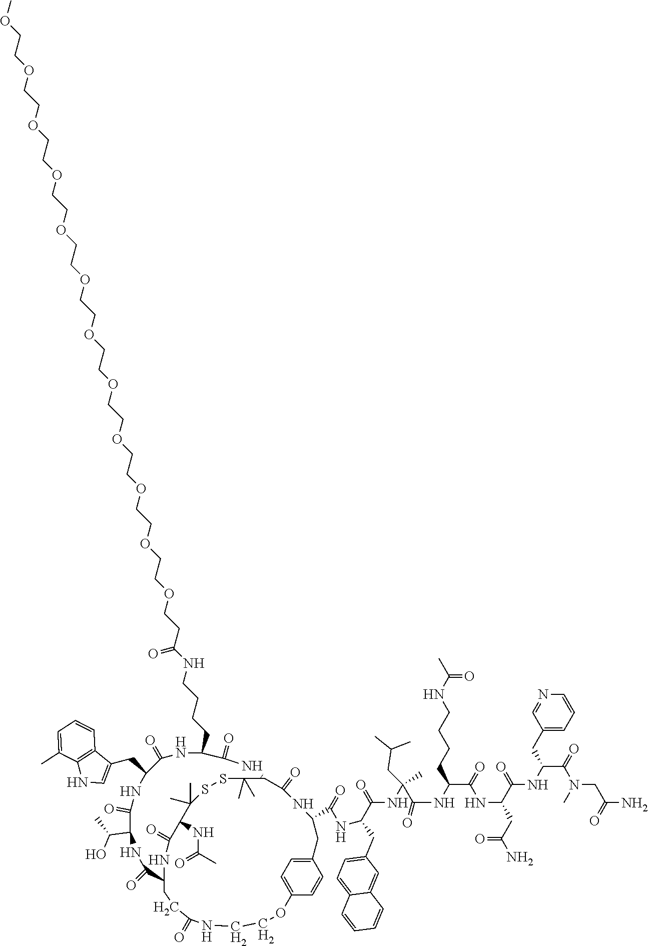 [SMILES: COCCOCCOCCOCCOCCOCCOCCOCCOCCOCCOCCOCCC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CCCCNC(C)=O)C(=O)N[C@@H](CC(N)=O)C(=O)N[C@H](Cc3cccnc3)C(=O)N(C)CC(N)=O)NC(=O)[C@@H](NC1=O)C(C)(C)SSC(C)(C)[C@H](NC(C)=O)C(=O)N2] | |
| 138 | 121 | Ac-[Pen]-E-T-[W(7-Me)]-[Lys(Ac_Morph)]-[Pen]-[Phe(4-(2- | #N/A |
| | | aminoethoxy))]-[2Nal]-[aMeLeu]-L-N-[3Pal]-[Sarc]-NH2 | |
| | | 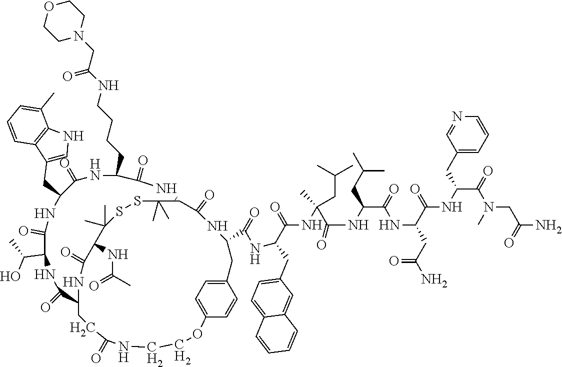 [SMILES: CC(=O)N[C@@H]1C(=O)N[C@@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CC(C)C)C(=O)N[C@@H](CC(N)=O)C(=O)N[C@H](Cc3cccnc3)C(=O)N(C)CC(N)=O)NC(=O)[C@@H](NC(=O)[C@H](CCCCNC(=O)CN3CCOCC3)NC(=O)[C@H](Cc3c[nH]c4c(C)cccc34)NC(=O)[C@H]([C@@H](C)O)NC2=O)C(C)(C)SSC1(C)C] | |

wherein Pen-Pen form a disulfide bond, or Abu-C form a thioether bond.

TABLE 1G
Compounds

| SEQ ID. | Cmpd. | Sequence/Structure |
|---|---|---|
| 139 | 122 | Ac-[Pen]-E-T-[W(7-Me)]-[Lys(Ac_Morph)]-[Pen]-[Phe(4-(2- |
| | | aminoethoxy))]-[2Nal]-[aMeLeu]-[Lys(Ac)]-L-[3Pal]-[Sarc]-NH2 |
| | | 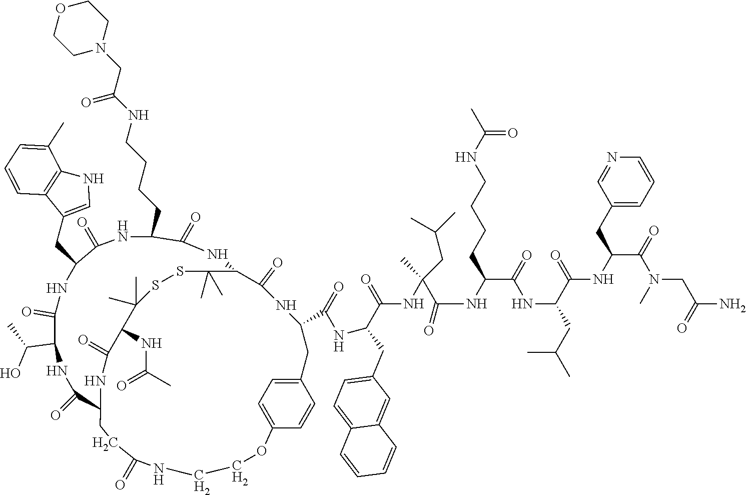 [SMILES: CC(=O)NCCCC[C@H](NC(=O)[C@](C)(CC(C)C)NC(=O)[C@H](Cc1ccc2ccccc2c1)NC(=O)[C@@H]1Cc2ccc(cc2)OCCNC(=O)CC[C@H]2NC(=O)[C@@H](NC(C)=O)C(C)(C)SSC(C)(C)[C@H](NC(=O)[C@H](CCCCNC(=O)CN3CCOCC3)NC(=O)[C@H](Cc3c[nH]c4c(C)cccc34)NC(=O)[C@H]([C@@H](C)O)NC2=O)C(=O)N1)C(=O)N[C@@H](CC(C)C)C(=O)N[C@@H](Cc1cccnc1)C(=O)N(C)CC(N)=O] |
| 140 | 123 | Ac-[Pen]-E-T-[W(7-Me)]-[Lys(Ac_Morph)]-[Pen]-[Phe(4-(2- |
| | | aminoethoxy))]-[2Nal]-[THP]-[Lys(Ac)]-L-[3Pal]-[Sarc]-NH2 |
| | | 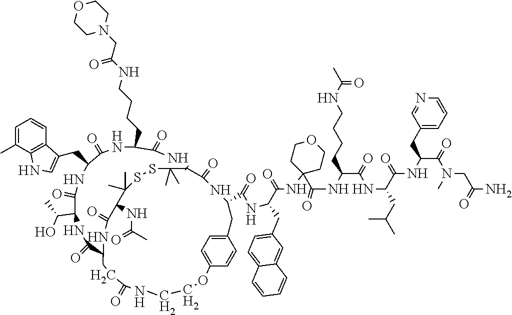 [SMILES: CC(=O)NCCCC[C@H](NC(=O)C1(NC(=O)[C@H](Cc2ccc3ccccc3c2)NC(=O)[C@@H]2Cc3ccc(cc3)OCCNC(=O)CC[C@H]3NC(=O)[C@@H](NC(C)=O)C(C)(C)SSC(C)(C)[C@H](NC(=O)[C@H](CCCCNC(=O)CN4CCOCC4)NC(=O)[C@H](Cc4c[nH]c5c(C)cccc45)NC(=O)[C@H]([C@@H](C)O)NC3=O)C(=O)N2)CCOCC1)C(=O)N[C@@H](CC(C)C)C(=O)N[C@@H](Cc1cccnc1)C(=O)N(C)CC(N)=O] |
| 141 | 124 | Ac-[Pen]-E-T-[W(7-Me)]-[Lys(Ac_Morph)]-[Pen]-[Phe(4-(2- |
| | | aminoethoxy))]-[2Nal]-[aMeLeu]-[Lys(Ac)]-N-[3Pal]-[Sarc]-NH2 |
| | | 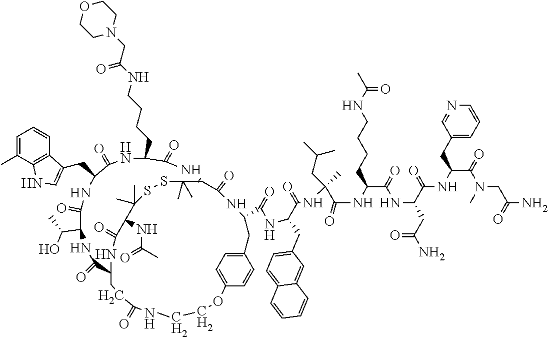 [SMILES: CC(=O)NCCCC[C@H](NC(=O)[C@](C)(CC(C)C)NC(=O)[C@H](Cc1ccc2ccccc2c1)NC(=O)[C@@H]1Cc2ccc(cc2)OCCNC(=O)CC[C@H]2NC(=O)[C@@H](NC(C)=O)C(C)(C)SSC(C)(C)[C@H](NC(=O)[C@H](CCCCNC(=O)CN3CCOCC3)NC(=O)[C@H](Cc3c[nH]c4c(C)cccc34)NC(=O)[C@H]([C@@H](C)O)NC2=O)C(=O)N1)C(=O)N[C@@H](CC(N)=O)C(=O)N[C@@H](Cc1cccnc1)C(=O)N(C)CC(N)=O] |
| 142 | 125 | [PEG12_OMe]-[Pen]-E-T-[W(7-Me)]-[Lys(Ac)]-[Pen]-[Phe(4-(2- |
| | | aminoethoxy))]-[2Nal]-[THP]-[Lys(Ac)]-N-[3Pal]-[Sarc]-NH2 |
| | | 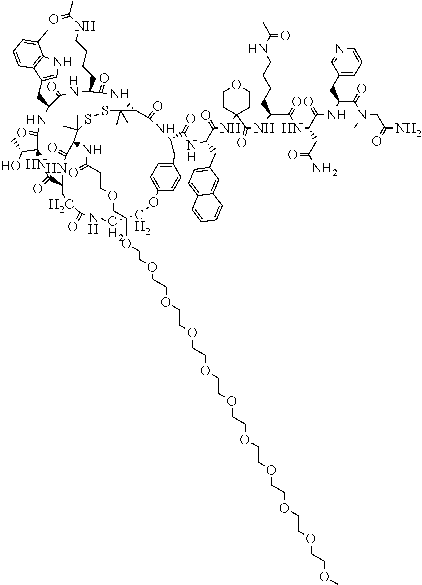 [SMILES: COCCOCCOCCOCCOCCOCCOCCOCCOCCOCCOCCOCCC(=O)N[C@H]1C(=O)N[C@@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC(=O)[C@H](CCCCNC(C)=O)NC(=O)[C@H](Cc3c[nH]c4c(C)cccc34)NC(=O)[C@H]([C@@H](C)O)NC2=O)C(C)(C)SSC1(C)C] |
| 143 | 126 | [PEG12_OMe]-[Pen]-E-L-[W(7-Me)]-[Lys(Ac)]-[Pen]-[Phe(4-(2- |
| | | aminoethoxy))]-[2Nal]-[THP]-L-N-[3Pal]-[Sarc]-NH2 |
| | | 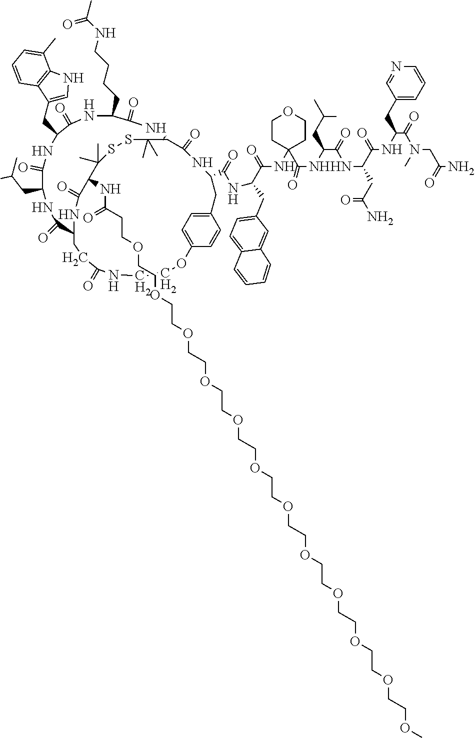 [SMILES: COCCOCCOCCOCCOCCOCCOCCOCCOCCOCCOCCOCCC(=O)N[C@@H]1C(=O)N[C@@H]2CCC(=O)NCCOc3ccc(cc3)C[C@@H](C(=O)N[C@@H](Cc3ccc4ccccc4c3)C(=O)NC3(C(=O)N[C@@H](CC(C)C)C(=O)N[C@@H](CC(N)=O)C(=O)N[C@@H](Cc4cccnc4)C(=O)N(C)CC(N)=O)CCOCC3)NC(=O)[C@@H](NC(=O)[C@H](CCCCNC(C)=O)NC(=O)[C@H](Cc3c[nH]c4c(C)cccc34)NC(=O)[C@H](CC(C)C)NC2=O)C(C)(C)SSC1(C)C] |
| 144 | 127 | [PEG12_OMe]-[Pen]-E-T-[W(7-Me)]-[Lys(Ac)]-[Pen]-[Phe(4-(2- |
| | | aminoethoxy))]-[2Nal]-[aMeLeu]-L-N-[3Pal]-[Sarc]-NH2 |
| | | 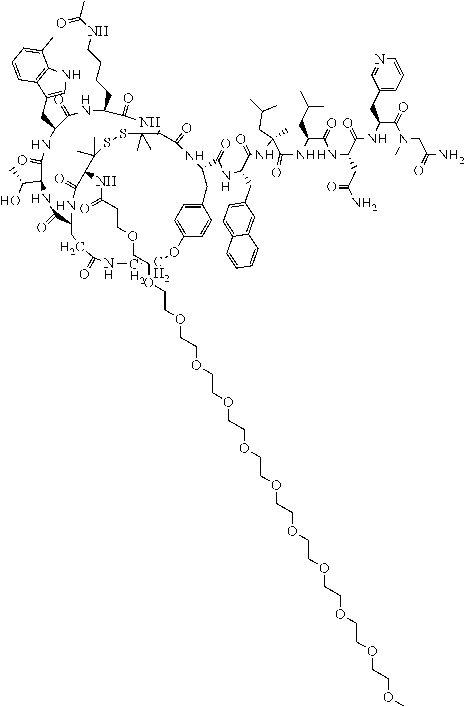 [SMILES: COCCOCCOCCOCCOCCOCCOCCOCCOCCOCCOCCOCCC(=O)N[C@@H]1C(=O)N[C@@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CC(C)C)C(=O)N[C@@H](CC(N)=O)C(=O)N[C@@H](Cc3cccnc3)C(=O)N(C)CC(N)=O)NC(=O)[C@@H](NC(=O)[C@H](CCCCNC(C)=O)NC(=O)[C@H](Cc3c[nH]c4c(C)cccc34)NC(=O)[C@H]([C@@H](C)O)NC2=O)C(C)(C)SSC1(C)C] |
| 145 | 128 | [PEG12_OMe]-[Pen]-E-T-[W(7-Me)]-[Lys(Ac)]-[Pen]-[Phe(4-(2- |
| | | aminoethoxy))]-[2Nal]-[aMeLeu]-[Lys(Ac)]-L-[3Pal]-[Sarc]-NH2 |
| | | 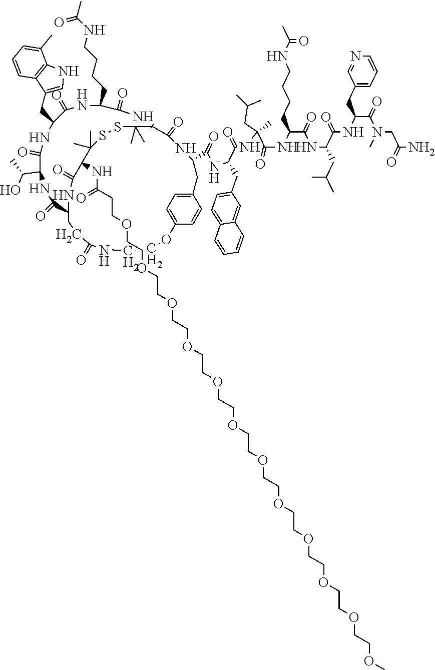 [SMILES: COCCOCCOCCOCCOCCOCCOCCOCCOCCOCCOCCOCCC(=O)N[C@@H]1C(=O)N[C@@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CCCCNC(C)=O)C(=O)N[C@@H](CC(C)C)C(=O)N[C@@H](Cc3cccnc3)C(=O)N(C)CC(N)=O)NC(=O)[C@@H](NC(=O)[C@H](CCCCNC(C)=O)NC(=O)[C@H](Cc3c[nH]c4c(C)cccc34)NC(=O)[C@H]([C@@H](C)O)NC2=O)C(C)(C)SSC1(C)C] |
| 146 | 129 | [PEG12_OMe]-[Pen]-E-T-[W(7-Me)]-[Lys(Ac)]-[Pen]-[Phe(4-(2- |
| | | aminoethoxy))]-[2Nal]-[THP]-[Lys(Ac)]-L-[3Pal]-[Sarc]-NH2 |
| | | 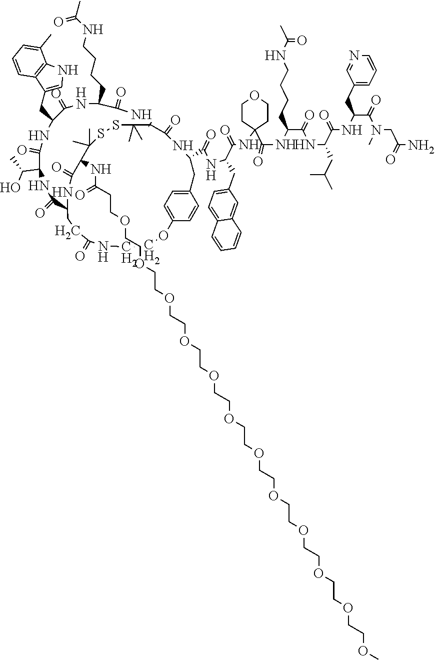 [SMILES: COCCOCCOCCOCCOCCOCCOCCOCCOCCOCCOCCOCCC(=O)N[C@@H]1C(=O)N[C@@H]2CCC(=O)NCCOc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(C)C)C(=O)N[C@@H](Cc4cccnc4)C(=O)N(C)CC(N)=O)CCOCC3)NC(=O)[C@@H](NC(=O)[C@H](CCCCNC(C)=O)NC(=O)[C@H](Cc3c[nH]c4c(C)cccc34)NC(=O)[C@H]([C@@H](C)O)NC2=O)C(C)(C)SSC1(C)C] |
| 147 | 130 | [Ac_Morph]-[Pen]-E-L-[W(7-Me)]-[Lys(Ac)]-[Pen]-[Phe(4-(2- |
| | | aminoethoxy))]-[2Nal]-[aMeLeu]-L-N-[3Pal]-[Sarc]-NH2 |
| | | 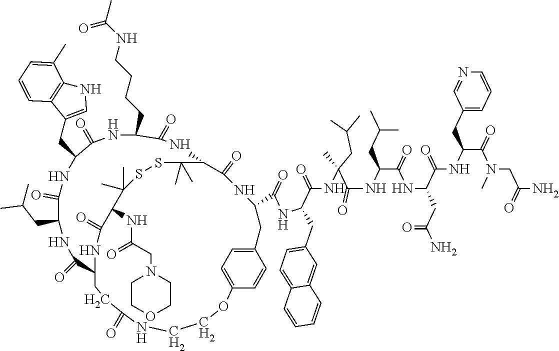 [SMILES: CC(=O)NCCCC[C@@H]1NC(=O)[C@H](Cc2c[nH]c3c(C)cccc23)NC(=O)[C@H](CC(C)C)NC(=O)[C@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CC(C)C)C(=O)N[C@@H](CC(N)=O)C(=O)N[C@@H](Cc3cccnc3)C(=O)N(C)CC(N)=O)NC(=O)[C@@H](NC1=O)C(C)(C)SSC(C)(C)[C@H](NC(=O)CN1CCOCC1)C(=O)N2] |
| 148 | 131 | [Ac_Morph]-[Pen]-E-L-[W(7-Me)]-[Lys(Ac)]-[Pen]-[Phe(4-(2- |
| | | aminoethoxy))]-[2Nal]-[THP]-L-N-[3Pal]-[Sarc]-NH2 |
| | | 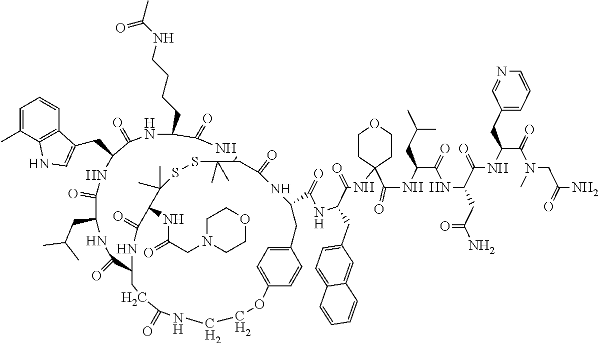 [SMILES: CC(=O)NCCCC[C@@H]1NC(=O)[C@H](Cc2c[nH]c3c(C)cccc23)NC(=O)[C@H](CC(C)C)NC(=O)[C@H]2CCC(=O)NCCOc3ccc(cc3)C[C@@H](C(=O)N[C@@H](Cc3ccc4ccccc4c3)C(=O)NC3(C(=O)N[C@@H](CC(C)C)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC(C)(C)[C@H](NC(=O)CN1CCOCC1)C(=O)N2] |
| 149 | 132 | [Ac_Morph]-[Pen]-E-T-[W(7-Me)]-[Lys(Ac)]-[Pen]-[Phe(4-(2- |
| | | aminoethoxy))]-[2Nal]-[aMeLeu]-[Lys(Ac)]-L-[3Pal]-[Sarc]-NH2 |
| | | 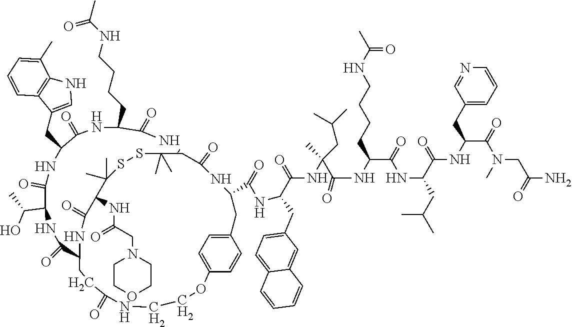 [SMILES: CC(=O)NCCCC[C@@H]1NC(=O)[C@H](Cc2c[nH]c3c(C)cccc23)NC(=O)[C@H]([C@@H](C)O)NC(=O)[C@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CCCCNC(C)=O)C(=O)N[C@@H](CC(C)C)C(=O)N[C@@H](Cc3cccnc3)C(=O)N(C)CC(N)=O)NC(=O)[C@@H](NC1=O)C(C)(C)SSC(C)(C)[C@H](NC(=O)CN1CCOCC1)C(=O)N2] |
| 150 | 133 | [Ac_Morph]-[Pen]-E-T-[W(7-Me)]-[Lys(Ac)]-[Pen]-[Phe(4-(2- |
| | | aminoethoxy))]-[2Nal]-[aMeLeu]-[Lys(Ac)]-N-[3Pal]-[Sarc]-NH2 |
| | | 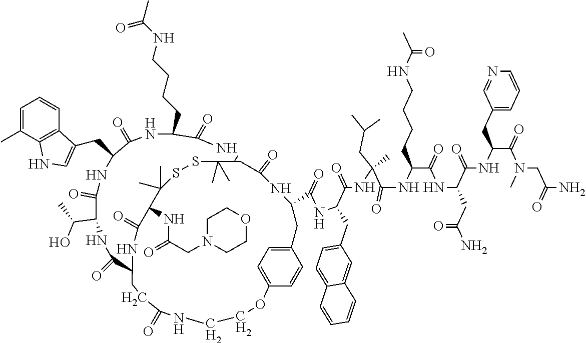 [SMILES: CC(=O)NCCCC[C@@H]1NC(=O)[C@H](Cc2c[nH]c3c(C)cccc23)NC(=O)[C@@H]([C@@H](C)O)NC(=O)[C@H]2CCC(=O)NCCOc3ccc(cc3)C[C@@H](C(=O)N[C@@H](Cc3ccc4ccccc4c3)C(=O)N[C@@](C)(CC(C)C)C(=O)N[C@@H](CCCCNC(C)=O)C(=O)N[C@@H](CC(N)=O)C(=O)N[C@@H](Cc3cccnc3)C(=O)N(C)CC(N)=O)NC(=O)[C@@H](NC1=O)C(C)(C)SSC(C)(C)[C@H](NC(=O)CN1CCOCC1)C(=O)N2] |
| 151 | 134 | Ac-E-[Pen]-N-T-[W(7-Me)]-[Lys(Ac)]-[Pen]-[Phe(4-(2-aminoethoxy))]- |
| | | [2Nal]-[THP]-[Lys(Ac)]-N-[3Pal]-[Sarc]-NH2 |
| | | 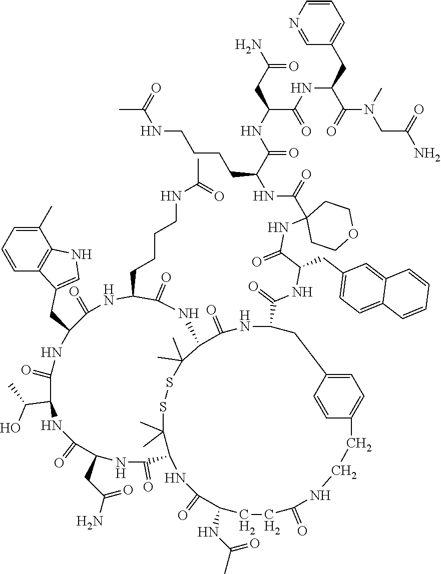 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)[C@@H](NC(C)=O)CCC(=O)NCCc3ccc(cc3)C[C@@H](C(=O)N[C@@H](Cc3ccc4ccccc4c3)C(=O)NC3(C(=O)N[C@@H](CCCCNC(C)=O)C(=O)N[C@@H](CC(N)=O)C(=O)N[C@@H](Cc4cccnc4)C(=O)N(C)CC(N)=O)CCOCC3)NC(=O)[C@@H](NC1=O)C(C)(C)SSC2(C)C] |
| 152 | 135 | Ac-E-[Pen]-N-T-[W(7-Me)]-[Lys(Ac)]-[Pen]-[Phe(4-OMe)]-[2Nal]-[THP]- |
| | | [Lys(Ac)]-N-[3Pal]-[(D)Lys]-NH2 |
| | | 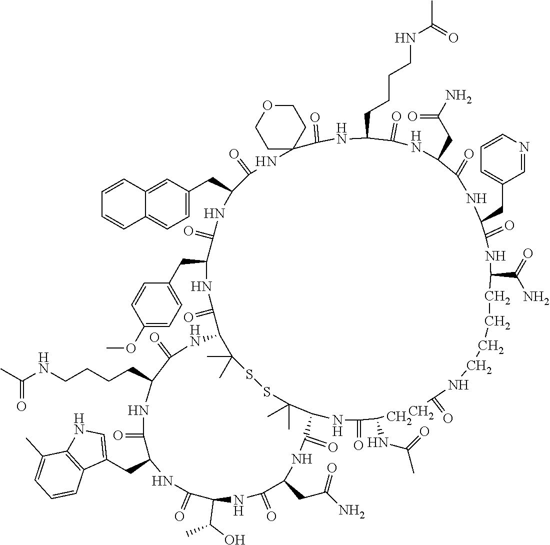 [SMILES: COc1ccc(C[C@H](NC(=O)[C@H]2NC(=O)[C@H](CCCCNC(C)=O)NC(=O)[C@H](Cc3c[nH]c4c(C)cccc34)NC(=O)[C@H]([C@@H](C)O)NC(=O)[C@H](CC(N)=O)NC(=O)[C@@H](NC(=O)[C@H](C)NC(C)=O)C(C)(C)SSC2(C)C)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N[C@H](CCCCNC(C)=O)C(N)=O)CCOCC2)cc1] |
| 153 | 136 | Ac-[(D)Lys]-A-E-T-[W(7-Me)]-[Lys(Ac)]-A-[Phe(4-(2-aminoethoxy))]- |
| | | [2Nal]-[THP]-[Lys(Ac)]-N-[3Pal]-[(D)Lys]-[NH2]-[PEG2DA] |
| | | 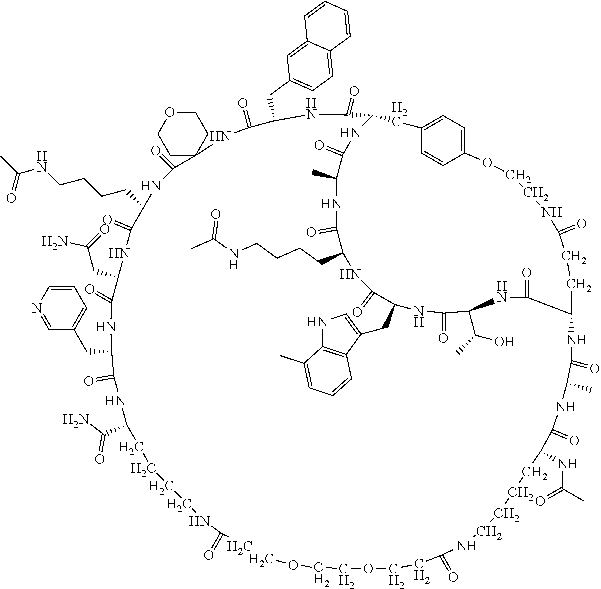 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@H](NC(=O)[C@H](C)NC1=O)C(=O)N[C@@H](Cc1ccc3ccccc3c1)C(=O)NC1(CCOCC1)C(=O)N[C@H](CCCCNC(C)=O)C(=O)N[C@H](CC(N)=O)C(=O)N[C@@H](Cc1cccnc1)C(=O)N[C@@H](C(N)=O)CCCCNC(=O)CCOCCOCCC(=O)NCCCC[C@@H](NC(C)=O)C(=O)N[C@@H](C)C(=O)N2] |
| 154 | 137 | [7AminoHeptanoicAcid]-[Pen]-N-T-[W(7-Me)]-[Lys(Ac)]-[Pen]-[Phe(4-(2- |
| | | aminoethoxy))]-[2Nal]-[THP]-E-N-[3Pal]-[Sarc]-NH2 |
| | | 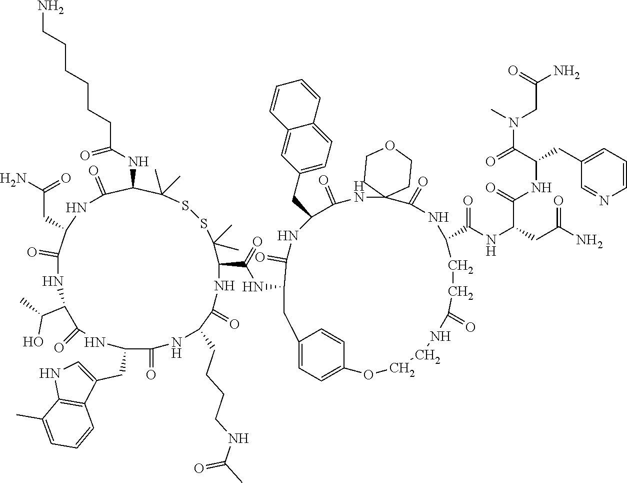 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCCCCCN)C(C)(C)SSC(C)(C)[C@@H](C(=O)N[C@H]2Cc3ccc(cc3)OCCNC(=O)CC[C@@H](C(=O)N[C@@H](CC(N)=O)C(=O)N[C@@H](Cc3cccnc3)C(=O)N(C)CC(N)=O)NC(=O)C3(CCOCC3)NC(=O)[C@H](Cc3ccc4ccccc4c3)NC2=O)NC1=O] |
| 155 | 138 | [7AminoHeptanoicAcid]-[Pen]-N-T-[W(7-Me)]-[Lys(Ac)]-[Pen]-[Phe(4-(2- |
| | | aminoethoxy))]-[2Nal]-[THP]-E-N-[(D)NMeTyr]-NH2 |
| | | 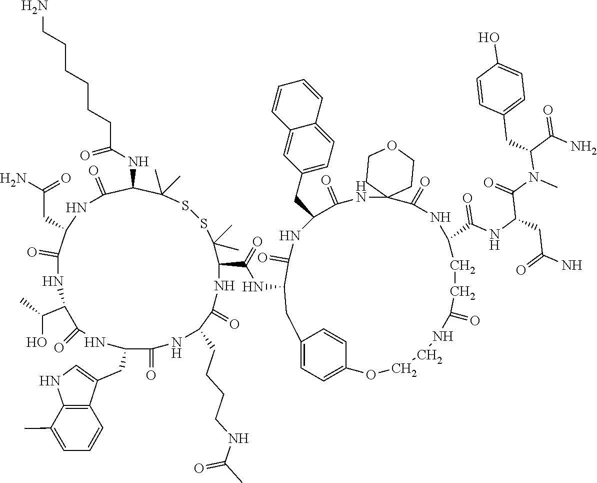 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCCCCCN)C(C)(C)SSC(C)(C)[C@@H](C(=O)N[C@H]2Cc3ccc(cc3)OCCNC(=O)CC[C@@H](C(=O)N[C@@H](CC(N)=O)C(=O)N(C)[C@H](Cc3ccc(O)cc3)C(N)=O)NC(=O)C3(CCOCC3)NC(=O)[C@H](Cc3ccc4ccccc4c3)NC2=O)NC1=O] |
| 156 | 139 | [7AminoHeptanoic Acid]-[Pen]-N-T-[W(7-Me)]-[Lys(Ac)]-[Pen]-[Phe(4-(2- |
| | | aminoethoxy))]-[2Nal]-[THP]-E-N-[THP]-NH2 |
| | | 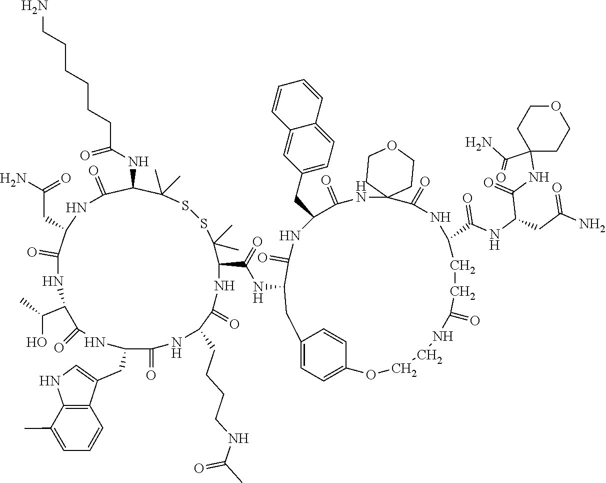 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCCCCCN)C(C)(C)SSC(C)(C)[C@@H](C(=O)N[C@H]2Cc3ccc(cc3)OCCNC(=O)CC[C@@H](C(=O)N[C@@H](CC(N)=O)C(=O)NC3(C(N)=O)CCOCC3)NC(=O)C3(CCOCC3)NC(=O)[C@H](Cc3ccc4ccccc4c3)NC2=O)NC1=O] |
| 157 | 140 | Ac-E-A-E-T-[W(7-Me)]-[Lys(Ac)]-A-[Phe(4-(2-aminoethoxy))]-[2Nal]- |
| | | [THP]-[Lys(Ac)]-N-[(D)Lys]-NH2 |
| | | 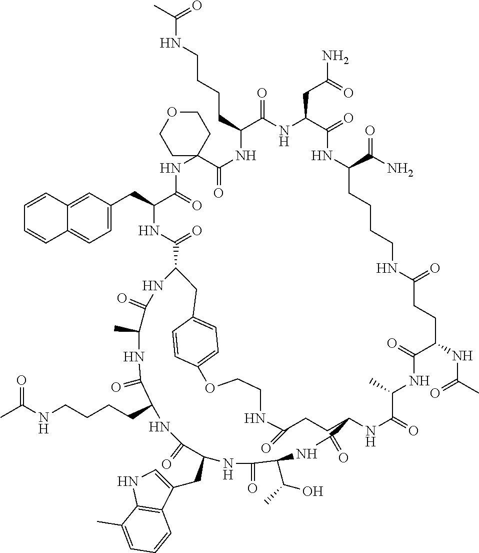 [SMILES: CC(=O)NCCCC[C@@H]1NC(=O)[C@H](Cc2c[nH]c3c(C)cccc23)NC(=O)[C@H]([C@@H](C)O)NC(=O)[C@@H]2CCC(=O)NCCOc3ccc(cc3)C[C@H](NC(=O)[C@H](C)NC1=O)C(=O)N[C@@H](Cc1ccc3ccccc3c1)C(=O)NC1(CCOCC1)C(=O)N[C@@H](CCCCNC(C)=O)C(=O)N[C@@H](CC(N)=O)C(=O)N[C@@H](C(N)=O)CCCCNC(=O)CC[C@H](NC(C)=O)C(=O)N[C@@H](C)C(=O)N2] |
| 158 | 141 | Ac-[Pen]-E-T-[W(7-Me)]-[Lys(PEG12_OMe)]-[Pen]-[Phe(4-(2- |
| | | aminoethoxy))]-[2Nal]-[THP]-[Lys(Ac)]-L-[3Pal]-[Sarc]-NH2 |
| | | 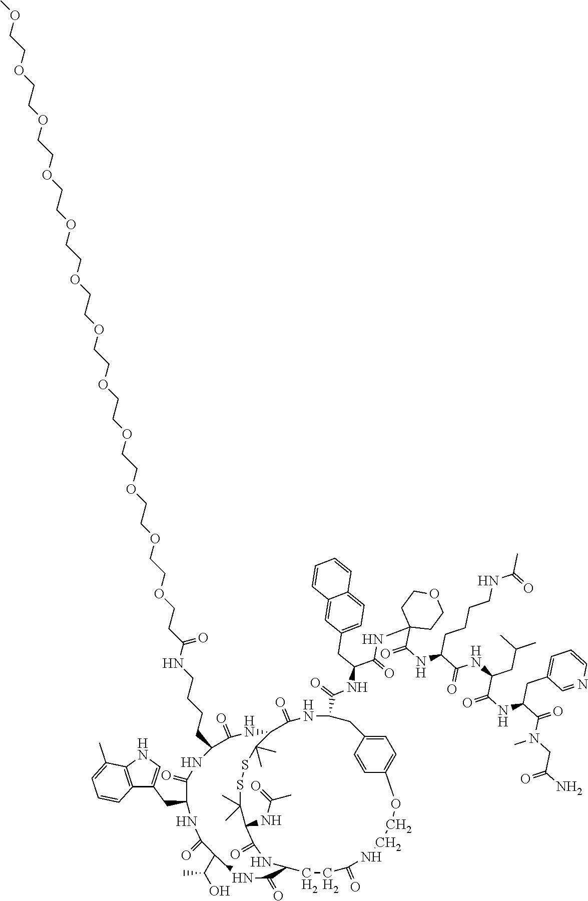 |
| 159 | 142 | Ac-[Pen]-E-T-[W(7-Me)]-[Lys(PEG12_OMe)]-[Pen]-[Phe(4-(2- |
| | | aminoethoxy))]-[2Nal]-[aMeLeu]-[Lys(Ac)]-N-[3Pal]-[Sarc]-NH2 |
| | | 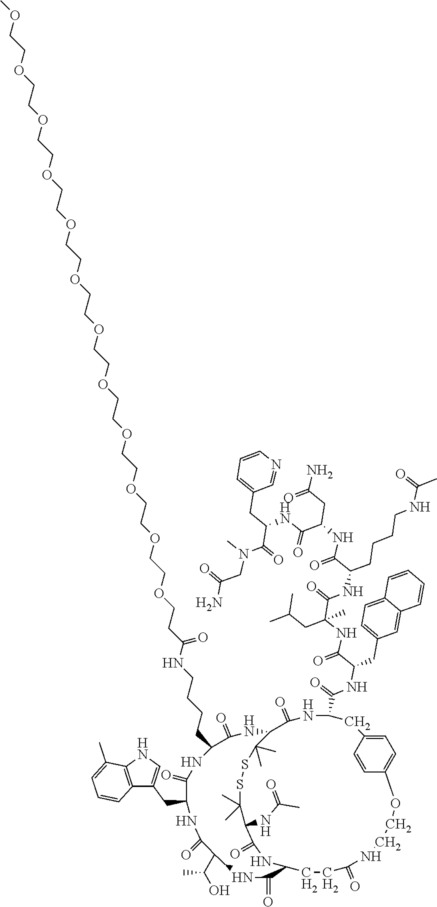 |
| 160 | 143 | Ac-[Pen]-E-T-[W(7-Me)]-[Lys(Ac_Morph)]-[Pen]-[Phe(4-(2- |
| | | aminoethoxy))]-[2Nal]-[aMeLeu]-[Lys(Ac)]-L-[3Pal]-[Sarc]-NH2 |
| | | 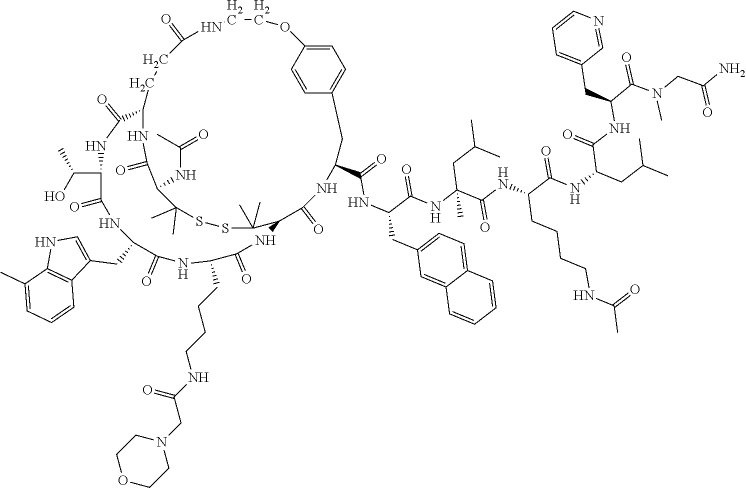 [SMILES: CC(=O)NCCCC[C@H](NC(=O)[C@](C)(CC(C)C)NC(=O)[C@H](Cc1ccc2ccccc2c1)NC(=O)[C@@H]1Cc2ccc(cc2)OCCNC(=O)CC[C@@H]2NC(=O)[C@@H](NC(C)=O)C(C)(C)SSC(C)(C)[C@H](NC(=O)[C@H](CCCCNC(=O)CN3CCOCC3)NC(=O)[C@H](Cc3c[nH]c4c(C)cccc34)NC(=O)[C@H]([C@@H](C)O)NC2=O)C(=O)N1)C(=O)N[C@@H](CC(C)C)C(=O)N[C@@H](Cc1cccnc1)C(=O)N(C)CC(N)=O] |
| 161 | 144 | Ac-[Pen]-E-T-[W(7-Me)]-[Lys(Ac_Morph)]-[Pen]-[Phe(4-(2- |
| | | aminoethoxy))]-[2Nal]-[THP]-[Lys(Ac)]-L-[3Pal]-[Sarc]-NH2 |
| | | 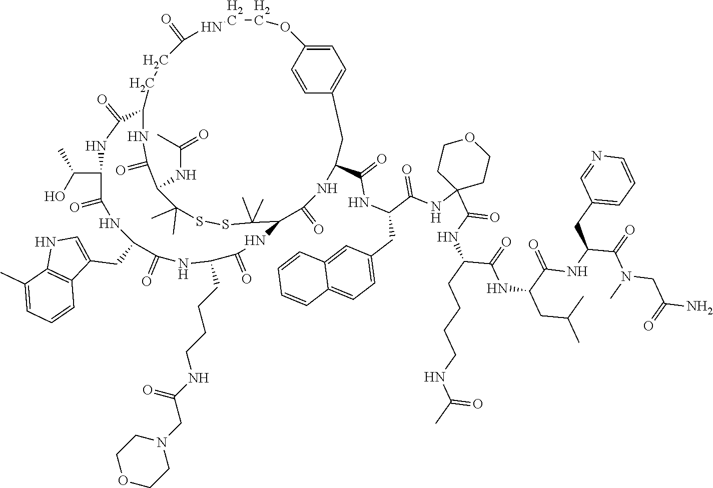 [SMILES: CC(=O)NCCCC[C@H](NC(=O)C1(NC(=O)[C@H](Cc2ccc3ccccc3c2)NC(=O)[C@@H]2Cc3ccc(cc3)OCCNC(=O)CC[C@@H]3NC(=O)[C@@H](NC(C)=O)C(C)(C)SSC(C)(C)[C@H](NC(=O)[C@H](CCCCNC(=O)CN4CCOCC4)NC(=O)[C@H](Cc4c[nH]c5c(C)cccc45)NC(=O)[C@H]([C@@H](C)O)NC3=O)C(=O)N2)CCOCC1)C(=O)N[C@@H](CC(C)C)C(=O)N[C@@H](Cc1cccnc1)C(=O)N(C)CC(N)=O] |
| 162 | 145 | Ac-[Pen]-E-T-[W(7-Me)]-[Lys(Ac_Morph)]-[Pen]-[Phe(4-(2- |
| | | aminoethoxy))]-[2Nal]-[aMeLeu]-[Lys(Ac)]-N-[3Pal]-[Sarc]-NH2 |
| | | 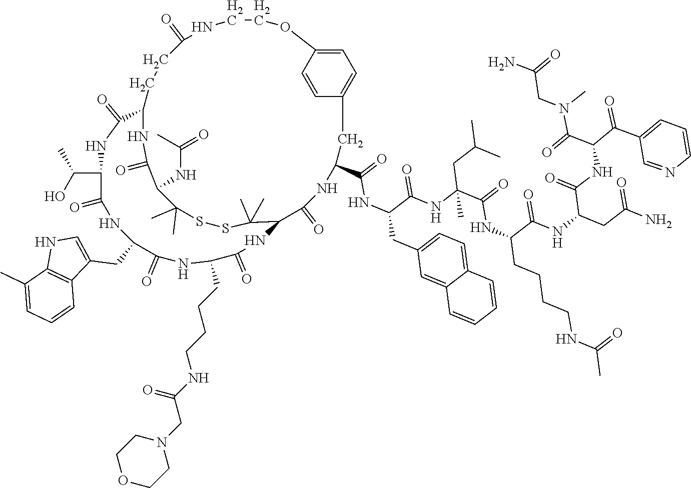 [SMILES: CC(=O)NCCCC[C@H](NC(=O)[C@](C)(CC(C)C)NC(=O)[C@H](Cc1ccc2ccccc2c1)NC(=O)[C@@H]1Cc2ccc(cc2)OCCNC(=O)CC[C@@H]2NC(=O)[C@@H](NC(C)=O)C(C)(C)SSC(C)(C)[C@H](NC(=O)[C@H](CCCCNC(=O)CN3CCOCC3)NC(=O)[C@H](Cc3c[nH]c4c(C)cccc34)NC(=O)[C@H]([C@@H](C)O)NC2=O)C(=O)N1)C(=O)N[C@@H](CC(N)=O)C(=O)N[C@@H](C(=O)c1cccnc1)C(=O)N(C)CC(N)=O] |

TABLE 1H
Compounds

| SEQ | | |
|---|---|---|
| ID | Structure | |
| NO | Peptide Sequence | Smiles Structure |
| 163 | 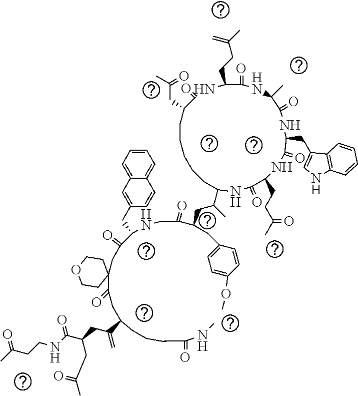 [SMILES: C=C(C)CC[C@@H]1NC(=O)[C@@H](CC(C)=O)CCCCCC(C(C)C[C@@H](Cc2ccc(OC)cc2)C(=O)CN[C@@H](Cc2ccc3ccccc3c2)C(=O)CC2(C(=O)C[C@@H](CCCC(=O)NC)C(=C)C[C@@H](CC(C)=O)C(=O)NCCC(C)=O)CCOCC2)NC(=O)[C@H](CCC(C)=O)NC(=O)[C@H](Cc2c[nH]c3ccccc23)NC(=O)[C@H](C)NC1=O] | C[C@H]([C@@H](C(N[C@@H](Cc1c [nH]c2c1cccc2)C(N[C@@H](CCC(N)═ O)C(N[C@@H](C(C)(C)SSC(C)(C)[C @@H\|(C(N[C@H]1CCC(N)═O)═O)NC (C)═O)C(N[C@@H](Cc(cc2)ccc2OCC NC(CC[C@@H](C(N[C@@H](CC(N)═ O)C(NCCC(N)═O)═O)═O)NC(C2(CCO CC2)NC([C@H](Cc2cc3ccccc3cc2)N2)═ O)═O)═O)C2═O)═O)═O)═O)═O) NC1═O)O |
| | MeCO-Pen(3)-Q-T-W-Q-Pen(3)-AEF(2)-2Nal- | |
| | THP-E(2)-N-bAla-CONH2 | |
| 164 | 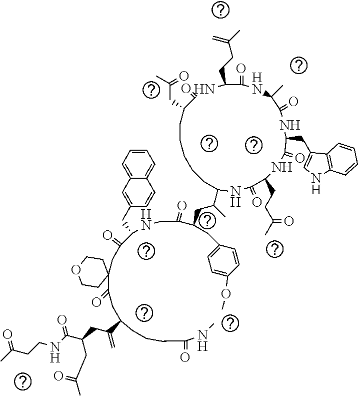 [SMILES: C=C(C)CC[C@@H]1NC(=O)[C@@H](CC(C)=O)CCCCCC(C(C)C[C@@H](Cc2ccc(OC)cc2)C(=O)CN[C@@H](Cc2ccc3ccccc3c2)C(=O)CC2(C(=O)C[C@@H](CCCC(=O)NC)C(=C)C[C@@H](CC(C)=O)C(=O)NCCC(C)=O)CCOCC2)NC(=O)[C@H](CCC(C)=O)NC(=O)[C@H](Cc2c[nH]c3ccccc23)NC(=O)[C@H](C)NC1=O] | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC(C)═O)C(N[C @@H\|(Cc(cc2)ccc2OCCNC(CC[C@@ H\|(C(N[C@@H](CC(N)═O)C(NCCC (N)═O)═O)═O)NC(C2 (CCOCC2)NC([C@ H](Cc2cc3ccccc3cc2)N2)═O)═O)═ O)C2═O)═O)═O)═O)═O)NC1═O)O |
| | MeCO-Abu(1)-Q-T-W-Q-C(1)-AEF(2)-2Nal-THP- | |
| | E(2)-N-bAla-CONH2 | |
| 165 | 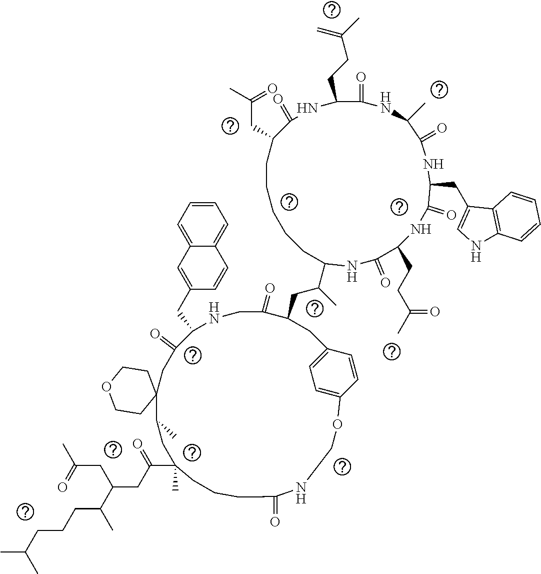 [SMILES: C=C(C)CC[C@@H]1NC(=O)[C@@H](CC(C)=O)CCCCCC(C(C)C[C@H]2Cc3ccc(cc3)OCNC(=O)CCC[C@@](C)(C(=O)CC(CC(C)=O)C(C)CCCC(C)C)C[C@H](C)C3(CCOCC3)CC(=O)[C@H](Cc3ccc4ccccc4c3)NCC2=O)NC(=O)[C@H](CCC(C)=O)NC(=O)[C@H](Cc2c[nH]c3ccccc23)NC(=O)[C@H](C)NC1=O] | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC(C)═O)C(N[C @@H](Cc(cc2)ccc2OCCNC(CC[C@@] (C)(C(N[C@@H](CC(N)═O)C(NCCC( N)═O)═O)═O)NC(C2(CCOCC2)NC([C @H](Cc2cc3ccccc3cc2)N2)═O)═O)═O) C2═O)═O)═O)═O)═O)NC1═O)O |
| | MeCO-Abu(1)-Q-T-W-Q-C(1)-AEF(2)-2Nal-THP- | |
| | aMeE(2)-N-bAla-CONH2 | |
| 166 | 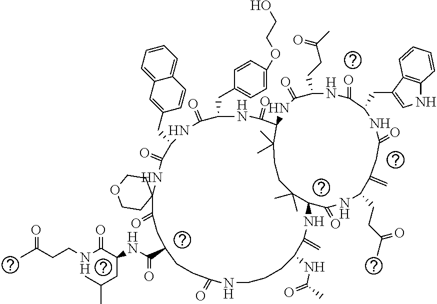 | C[C@H]([C@@H](C(N[C@@H](Cc1c [nH]c2c1cccc2)C(N[C@@H](CCC(N)═ O)C(N[C@@H](C(C)(C)SSC(C)(C)[C @@H](C(N[C@H]1CCC(N)═O)═O)NC ([C@@H](CCCCNC(CC[C@@H](C(N [C@@H](CC(N)═O)C(NCCC(N)═O)═ O)═O)NC(C2(CCOCC2)NC([C@H](Cc 2cc3ccccc3cc2)NC([C@H](Cc(cc2)ccc2 OCCN)N2)═O)═O)═O)═O)NC(C)═O)═ O)C2═O)═O)═O)═O)NC1═O)O |
| | MeCO-k(2)-Pen(3)-Q-T-W-Q-Pen(3)-AEF-2Nal- | |
| | THP-E(2)-N-bAla-CONH2 | |
| 167 | 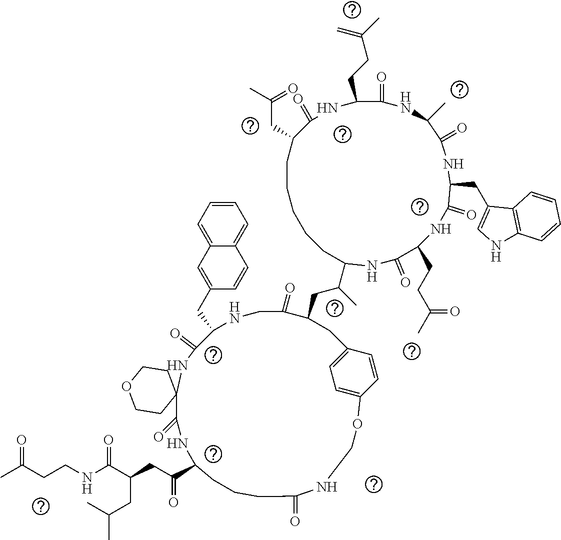 [SMILES: C=C(C)CC[C@@H]1NC(=O)[C@@H](CC(C)=O)CCCCCC(C(C)C[C@H]2Cc3ccc(cc3)OCNC(=O)CCC[C@@H](C(=O)C[C@@H](CC(C)C)C(=O)NCCC(C)=O)NC(=O)C3(CCOCC3)NC(=O)[C@H](Cc3ccc4ccccc4c3)NCC2=O)NC(=O)[C@H](CCC(C)=O)NC(=O)[C@H](Cc2c[nH]c3ccccc23)NC(=O)[C@H](C)NC1=O] | C[C@H][C@@H](C(N[C@@H](Cc1c [nHc2c1cccc2)C(N[C@@H](CCC(N)═ O)C(N[C@@H](CSCC[C@@H](C(N[C @H]1CCC(N)═O)═O)NC(C)═O)C(N[C @@H](Cc(cc2)ccc2OCCNC(CCC[C@ @H](C(N[C@@H](CC(N)═O)C(NCCC (N)═O)═O)═O)NC(C2(CCOCC2)NC([C @H](Cc2cc3ccccc3cc2)N2)═O)═O)═O) C2═O)═O)═O)═O)═O)NC1═O)O |
| | MeCO-Abu(1)-Q-T-W-Q-C(1)-AEF(2)-2Nal-THP- | |
| | hE(2)-N-bAla-CONH2 | |
| 168 | 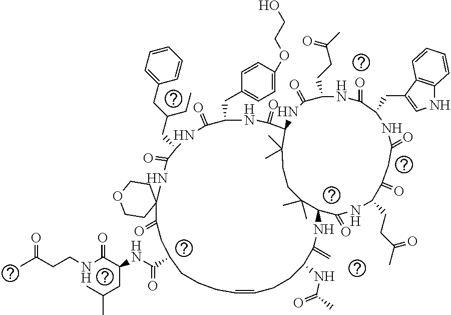 | C[C@H][C@@H](C(N[C@@H](Cc1c [nH]c2c1cccc2)C(N[C@@H](CCC(N)═ O)C(N[C@@H](C(C)(C)SSC(C)(C)[C @@H](C(N[C@H]1CCC(N)═O)═O)NC ([C@@H](CCC/C═C\CCC[C@H](C(N [C@@H](CC(N)═O)C(NCCC(N)═O)═ O)═O)NC(C2(CCOCC2)NC([C@H](Cc2c c3ccccc3cc2)NC([C@H](Cc(cc2)ccc2O CCN)N2)═O)═O)═O)NC(C)═O)═O)C2═ O)═O)═O)═O)NC1═O)O |
| | MeCO-R5H(4)-Pen(3)-Q-T-W-Q-Pen(3)-AEF- | |
| | 2Nal-THP-R5H(4)-N-bAla-CONH2 | |
| 169 | 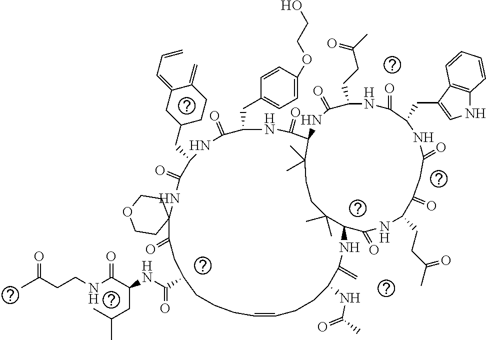 | C[C@H][C@@H](C(N[C@@H](Cc1c [nH]c2c1cccc2)C(N[C@@H](CCC(N)═ O)C(N[C@@H](C(C)(C)SSC(C)(C)[C @@H\|(C(N[C@H]1CCC(N)═O)═O)NC ([C@@H](CCC/C═C\CCC[C@H](C(N [C@@H\|(CC(N)═O)C(NCCC(N)═O)═ O)═O)NC(C2(CCOCC2)NC([C@H](Cc2c c3ccccc3cc2)NC([C@H](Cc(cc2)ccc2O CCN)N2)═O)═O)═O)NC(C)═O)═O)C2═ O)═O)═O)═O)NC1═O)O |
| | MeCO-R5H(4)-Pen(3)-Q-T-W-Q-Pen(3)-AEF- | |
| | 2Nal-THP-R5H(4)-N-bAla-CONH2 | |
| 170 | 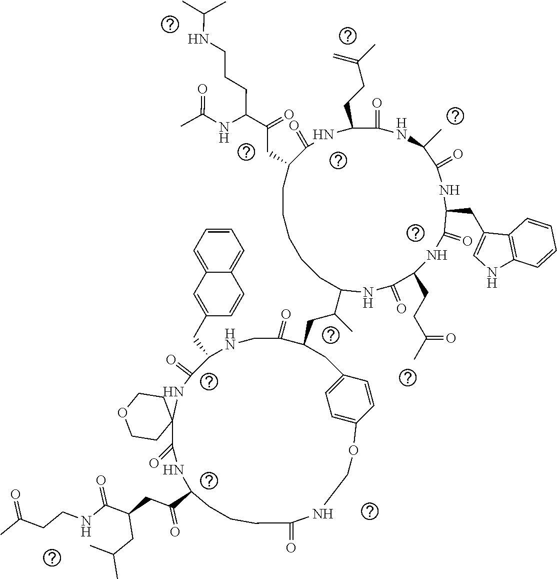 [SMILES: C=C(C)CC[C@@H]1NC(=O)[C@@H](CC(=O)C(CCCNC(C)C)NC(C)=O)CCCCCC(C(C)C[C@H]2Cc3ccc(cc3)OCNC(=O)CCC[C@@H](C(=O)C[C@@H](CC(C)C)C(=O)NCCC(C)=O)NC(=O)C3(CCOCC3)NC(=O)[C@H](Cc3ccc4ccccc4c3)NCC2=O)NC(=O)[C@H](CCC(C)=O)NC(=O)[C@H](Cc2c[nH]c3ccccc23)NC(=O)[C@H](C)NC1=O] | C[C@H][C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC([C@@H](CC CNC(N)═N)NC(C)═O)═O)C(N[C@@H] (Cc(cc2)ccc2OCCNC(CC[C@@H](C (N[C@@H](CC(N)═O)C(NCCC(N)═O)═ O)═O)NC(C2(CCOCC2)NC([C@H](C c2cc3ccccc3cc2)N2)═O)═O)═O)C2═O)═ O)═O)═O)═O)NC1═O)O |
| | MeCO-r-Abu(1)-Q-T-W-Q-C(1)-AEF(2)-2Nal- | |
| | THP-E(2)-N-bAla-CONH2 | |
| 171 | 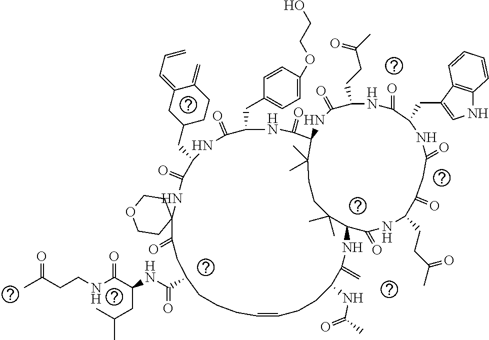 | C[C@H][C@@H](C(N[C@@H](Cc1c [nH]c2c1cccc2)C(N[C@@H](CCC(N)═ O)C(N[C@@H](CSCC[C@@H](C(N[C @@H](C(N[C@H]1CCC(N)═O)═O)NC ([C@@H](CCC/C═C\CCC[C@@H](C (N[C@@H](CC(N)═O)C(NCCC(N)═O)═ O)═O)NC(C2(CCOCC2)NC([C@H](C c2cc3ccccc3cc2)NC([C@H](Cc(cc2)ccc 2OCCN)N2)═O)═O)═O)NC(C)═O)═O) C2═O)═O)═O)═O)NC1═O)O |
| | MeCO-R5H(4)-Pen(3)-Q-T-W-Q-Pen(3)-AEF- | |
| | 2Nal-THP-S5H(4)-N-bAla-CONH2 | |
| 172 | 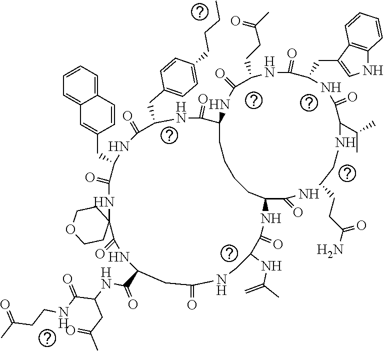 | C[C@H][C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC([C@@H](CN C(CC[C@@H](C(N[C@@H](CC(N)═O) C(NCCC(N)═O)═O)═O)NC(C2(CCOC C2)NC([C@H](Cc2cc3ccccc3cc2)NC ([C@H](Cc(cc2)ccc2OCCN)N2)═O)═O)═ O)═O)NC(C)═O)═O)C2═O)═O)═O)═O) NC1═O)O |
| | MeCO-dap(2)-Abu(1)-Q-T-W-Q-C(1)-AEF-2Nal- | |
| | THP-E(2)-N-bAla-CONH2 | |
| 173 | 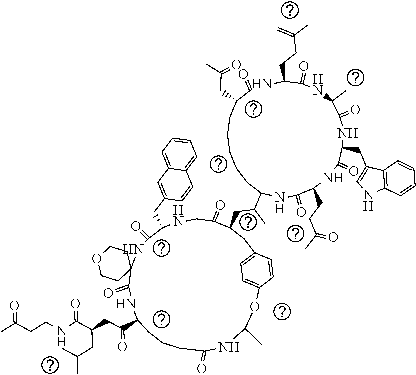 [SMILES: C=C(C)CC[C@@H]1NC(=O)[C@@H](CC(C)=O)CCCCCC(C(C)C[C@H]2Cc3ccc(cc3)OC(C)NC(=O)CCC[C@@H](C(=O)C[C@@H](CC(C)C)C(=O)NCCC(C)=O)NC(=O)C3(CCOCC3)NC(=O)[C@H](Cc3ccc4ccccc4c3)NCC2=O)NC(=O)[C@H](CCC(C)=O)NC(=O)[C@H](Cc2c[nH]c3ccccc23)NC(=O)[C@H](C)NC1=O] | C[C@H][C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC(C)═O)C(N[C @@H](Cc(cc2)ccc2OCC(NCCCC[C@ @H](C(N[C@@H](CC(N)═O)C(NCCC (N)═O)═O)═O)NC(C2(CCOCC2)NC([C @H](Cc2cc3ccccc3cc2)N2)═O)═O)═O) C2═O)═O)═O)═O)═O)NC1═O)O |
| | MeCO-Abu(1)-Q-T-W-Q-C(1)-CMF(2)-2Nal-THP- | |
| | K(2)-N-bAla-CONH2 | |
| 174 | 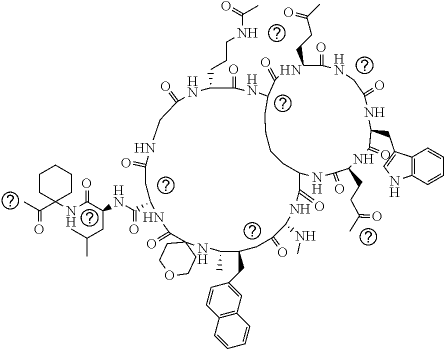 [SMILES: CN[C@H]1NC(=O)C2CCCCC(NC(=O)[C@@H](CCCNC(C)=O)NC(=O)CNC(=O)C[C@@H](C(=O)N[C@@H](CC(C)C)C(=O)NC3(C(C)=O)CCCCC3)NC(=O)C3(CCOCC3)N[C@@H](C)[C@H](Cc3ccc4ccccc4c3)CC1=O)C(=O)N[C@@H](CCC(C)=O)C(=O)NCC(=O)N[C@@H](Cc1c[nH]c3ccccc13)C(=O)N[C@@H](CCC(C)=O)C(=O)N2] | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC([C@@H](CC CNC(N)═N)NC(CCNC(CC[C@@H](C(N [C@@H](CC(N)═O)C(NC2(CCOCC2) C(N)═O)═O)═O)NC(C2(CCOCC2)NC( [C@H](Cc2cc3ccccc3cc2)NC([C@H](C c(cc2)ccc2OCCN)N2)═O)═O)═O)═O)═ O)═O)C2═O)═O)═O)═O)NC1═O)O |
| | bAla(2)-r-Abu(1)-Q-T-W-Q-C(1)-AEF-2Nal-THP- | |
| | E(2)-N-THP-CONH2 | |
| 175 | 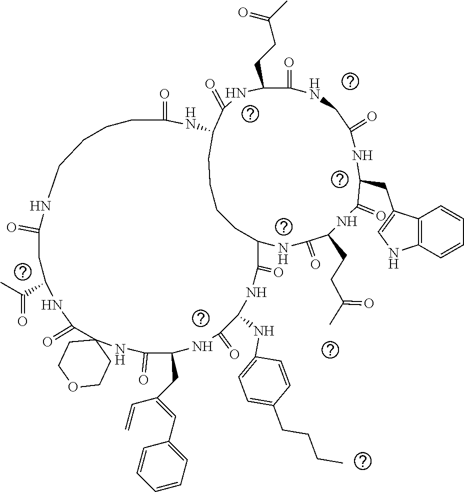 | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC(CCCCCNC(C C[C@@H](C(N)═O)NC(C2(CCOCC2) NC([C@H](Cc2cc3ccccc3cc2)NC([C@ H](Cc(cc2)ccc2OCCN)N2)═O)═O)═O)═ O)═O)C2═O)═O)═O)═O)NC1═O)O |
| | 6Ahx(2)-Abu(1)-Q-T-W-Q-C(1)-AEF-2Nal-THP- | |
| | E(2)-CONH2 | |
| 176 | 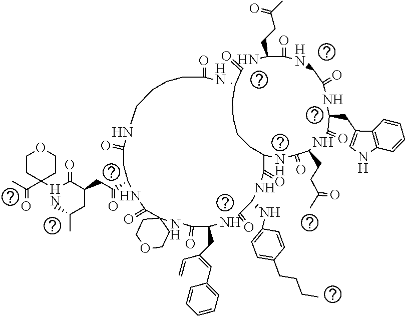 | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC(CCCCCNC(C C[C@@H](C(N[C@@H](CC(N)═O)C( NC2(CCOCC2)C(N)═O)═O)═O)NC(C2 (CCOCC2)NC([C@H](Cc2cc3ccccc3cc 2)NC([C@H](Cc(cc2)ccc2OCCN)N2)═ O)═O)═O)═O)═O)C2═O)═O)═O)═O)N C1═O)O |
| | 6Ahx(2)-Abu(1)-Q-T-W-Q-C(1)-AEF-2Nal-THP- | |
| | E(2)-N-THP-CONH2 | |
| 177 | 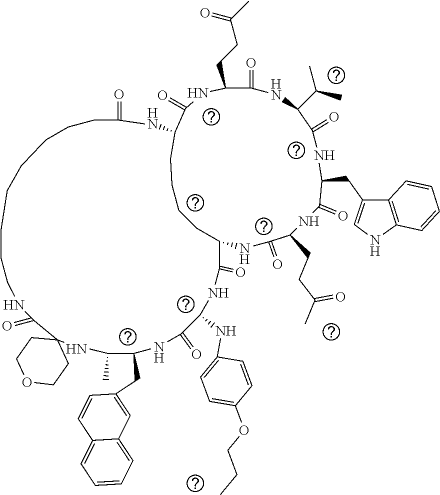 | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC(CCCCCCCN C(C2(CCOCC2)NC([C@H](Cc2cc3cccc c3cc2)NC([C@H](Cc(cc2)ccc2OCCN)N 2)═O)═O)═O)═O)C2═O)═O)═O)═O)NC 1═O)O |
| | 8Aoc(2)-Abu(1)-Q-T-W-Q-C(1)-AEF-2Nal-THP- | |
| | COOH(2) | |
| 178 | 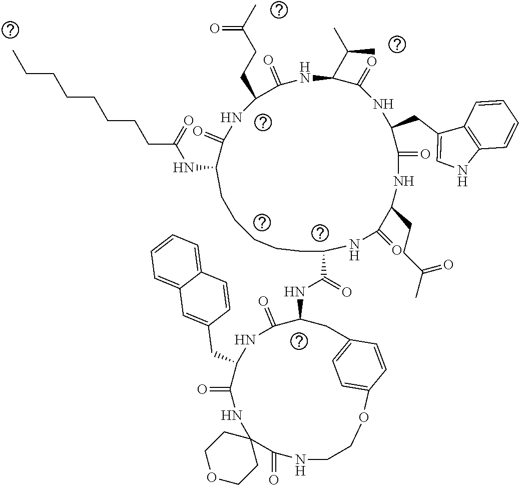 | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC(CCCCCCCN)═ O)C(N[C@@H](Cc(cc2)ccc2OCCNC (C2(CCOCC2)NC([C@H](Cc2cc3ccccc3 cc2)N2)═O)═O)C2═O)═O)═O)═O)═O)N C1═O)O |
| | 8Aoc-Abu(1)-Q-T-W-Q-C(1)-AEF(2)-2Nal-THP- | |
| | COOH(2) | |
| 179 | 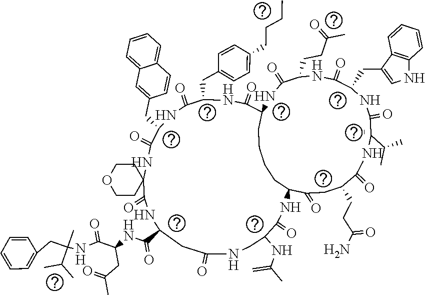 | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC([C@@H](CC CNC(CC[C@@H](C(N[C@@H](CC (N)═O)C(N[C@@](C)(Cc2ccccc2)C(N)═ O)═O)═O)NC(C2(CCOCC2)NC([C@H] (Cc2cc3ccccc3cc2)NC([C@H](Cc(cc2)cc c2OCCN)N2)═O)═O)═O)═O)NC(C)═ O)═O)C2═O)═O)═O)═O)NC1═O)O |
| | MeCO-orn(2)-Abu(1)-Q-T-W-Q-C(1)-AEF-2Nal- | |
| | THP-E(2)-N-aMePhe-CONH2 | |
| 180 | 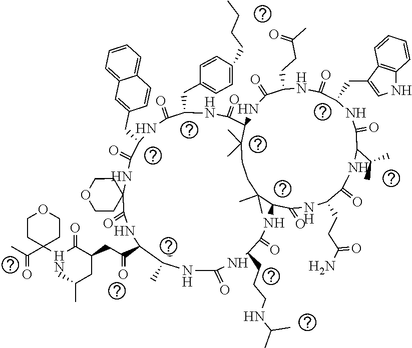 | C[C@H]([C@@H](C(N[C@@H](Cc1c [nH]c2c1cccc2)C(N[C@@H](CCC(N)═ O)C(N[C@@H](C(C)(C)SSC(C)(C)[C @@H](C(N[C@H]1CCC(N)═O)═O)NC ([C@@H](CCCNC(N)═N)NC(CCNC(C C[C@@H](C(N[C@@H](CC(N)═O)C (NC2(CCOCC2)C(N)═O)═O)═O)NC(C2 (CCOCC2)NC([C@H](Cc2cc3ccccc3cc 2)NC([C@H](Cc(cc2)ccc2OCCN)N2)═ O)═O)═O)═O)═O)═O)C2═O)═O)═O)═ O)NC1═O)O |
| | bA(2)-r-Pen(3)-Q-T-W-Q-Pen(3)-AEF-2Nal-THP- | |
| | E(2)-N-THP-CONH2 | |
| 181 | 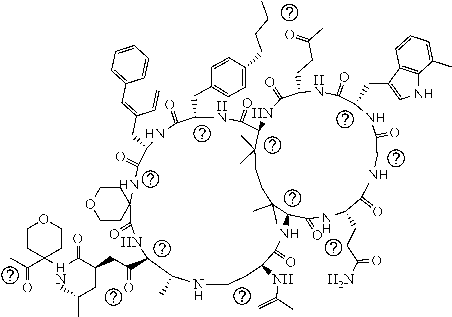 | C[C@H]([C@@H](C(N[C@@H](Cc1c [nH]c2c1cccc2)C(N[C@@H](CCC(N)═ O)C(N[C@@H](C(C)(C)SSC(C)(C)[C @@H](C(N[C@H]1CCC(N)═O)═O)NC ([C@H](CCCNC(CC[C@@H](C(N[C@ @H](CC(N)═O)C(NC2(CCOCC2)C (N)═O)═O)═O)NC(C2(CCOCC2)NC([C@ H](Cc2cc3ccccc3cc2)NC([C@H] (Cc(cc 2)ccc2OCCN)N2)═O)═O)═O)═O)NC (C)═O)═O)C2═O)═O)═O)═O)NC1═O)O |
| | MeCO-Orn(2)-Pen(3)-N-T-7MeW-Q-Pen(3)-AEF- | |
| | 2Nal-THP-E(2)-N-THP-CONH2 | |
| 182 | 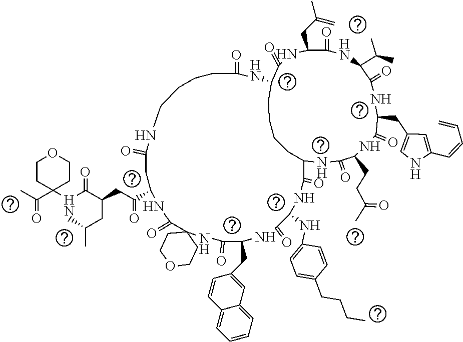 | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCCNC(CC [C@@H](C(N[C@@H](CC(N)═O)C(N C2(CCOCC2)C(N)═O)═O)═O)NC(C2(C COCC2)NC([C@H](Cc2cc3ccccc3cc2) NC([C@H](Cc(cc2)ccc2OCCN)N2)═ O)═O)═O)═O)═O)C2═O)═O)═O)═ O)NC1═O)O |
| | 6Ahx(2)-Abu(1)-N-T-W-Q-C(1)-AEF-2Nal-THP- | |
| | E(2)-N-THP-CONH2 | |
| 183 | 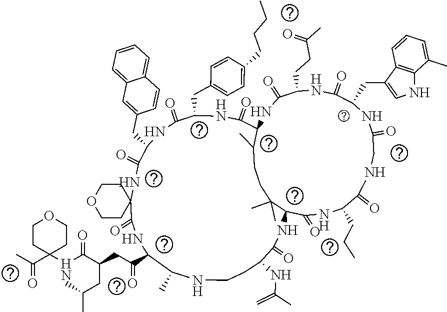 | C[C@H]([C@@H](C(N[C@@H](Cc1c [nH]c2c1cccc2C)C(N[C@@H](CCC(N)═ O)C(N[C@@H](C(C)(C)SSC(C)(C)[C @@H](C(N[C@H]1CC(N)═O)═O)NC ([C@@H](CCCNC(CC[C@@H](C(N[C @@H](CC(N)═O)C(NC2(CCOCC2)C (N)═O)═O)═O)NC(C2(CCOCC2)NC([C @H](Cc2cc3ccccc3cc2)NC([C@H] (Cc (cc2)ccc2OCCN)N2)═O)═O)═O)═O)NC (C)═O)═O)C2═O)═O)═O)═O)NC1═O)O |
| | MeCO-orn(2)-Pen(3)-N-T-7MeW-Q-Pen(3)-AEF- | |
| | 2Nal-THP-E(2)-N-THP-CONH2 | |
| 184 | 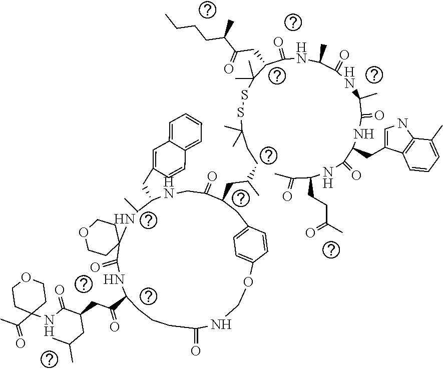 | C[C@H]([C@@H](C(N[C@@H](Cc1c [nH]c2c1cccc2C)C(N[C@@H](CCC(N)═ O)C(N[C@@H](C(C)(C)SSC(C)(C)[C @@H](C(N[C@H]1CC(N)═O)═O)NC ([C@@H](CCCN)NC(C)═O)═O)C(N[C @@H](Cc(cc2)ccc2OCCNC(CC[C@@ H](C(N[C@@H](CC(N)═O)C(NC2(CC OCC2)C(N)═O)═O)═O)NC(C2(CCOCC 2)NC([C@H](Cc2cc3ccccc3cc2)N2)═ O)═O)═O)C2═O)═O)═O)═O)═ O)NC1═O)O |
| | MeCO-orn-Pen(3)-N-T-7MeW-Q-Pen(3)-AEF(2)- | |
| | 2Nal-THP-E(2)-N-THP-CONH2 | |
| 185 | 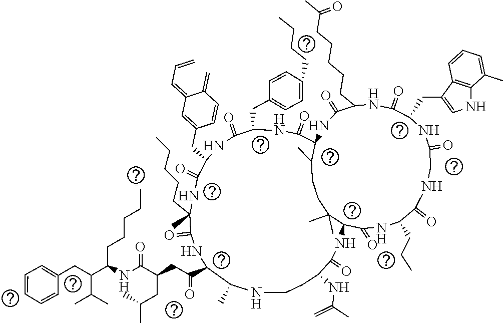 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCNC(CC[C@@H](C( N[C@@H](CC(N)═O)C(N[C@H](CCC CN)C(N(C)[C@H](Cc(cc2)ccc2O)C(N)═ O)═O)═O)═O)NC([C@](C)(CCCCN) NC([C@H](Cc2cc3ccccc3cc2)NC([C@ H](Cc(cc2)ccc2OCCN)N2)═O)═O)═ O)═O)NC(C)═O)═O)C2═O)═O)═O)═O) NC1═O)O |
| | MeCO-orn(2)-Pen(3)-N-T-7MeW-K(Ac)-Pen(3)- | |
| | AEF-2Nal-aMeK-E(2)-N-dK-NMeDTyr-CONH2 | |
| 186 | 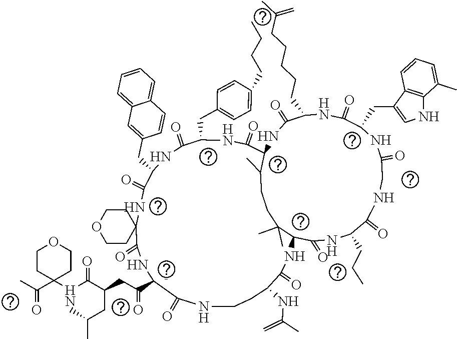 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCNC(CC[C@@H](C (N[C@@H](CC(N)═O)C(NC2(CCOCC2) C(N)═O)═O)═O)NC(C2(CCOCC2)NC ([C@H](Cc2cc3ccccc3cc2)NC([C@H](C c(cc2)ccc2OCCN)N2)═O)═O)═O)═O)N C(C)═O)═O)C2═O)═O)═O)═O)NC1═O) O |
| | MeCO-orn(2)-Pen(3)-N-T-7MeW-K(Ac)-Pen(3)- | |
| | AEF-2Nal-THP-E(2)-N-THP-CONH2 | |
| 187 | 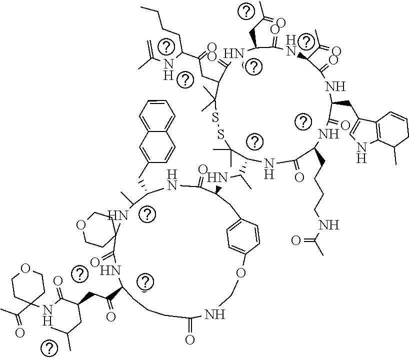 [SMILES: C=C(C)NC(CCCC)C(=O)CC1C(=O)N[C@@H](CC(C)=O)C(=O)N[C@@H](C(C)=O)C(=O)N[C@@H](Cc2c[nH]c3c2C=CCC3C)C(=O)N[C@@H](CCCCNC(C)=O)C(=O)N[C@H](C(C)N[C@H]2Cc3ccc(cc3)OCNC(=O)CCC[C@@H](C(=O)C[C@@H](CC(C)C)C(=O)NC3(C(C)=O)CCOCC3)NC(=O)C3(CCOCC3)NC(C)[C@H](Cc3ccc4ccccc4c3)NC2=O)C(C)(C)SSC1(C)C] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCN)NC(C)═O)═O)C (N[C@@H](Cc(cc2)ccc2OCCNC(CC[C @@H](C(N[C@@H](CC(N)═O)C(NC2 (CCOCC2)C(N)═O)═O)═O)NC(C2(CC OCC2)NC([C@H](Cc2cc3ccccc3cc2)N 2)═O)═O)═O)C2═O)═O)═O)═O)═O) NC1═O)O |
| | MeCO-orn-Pen(3)-N-T-7MeW-K(Ac)-Pen(3)- | |
| | AEF(2)-2Nal-THP-E(2)-N-THP-CONH2 | |
| 188 | 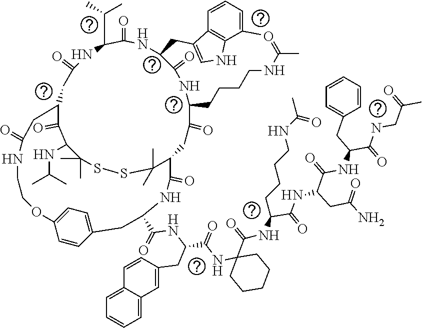 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCCCNC(C)═O)C(N[C@@H](CC (N)═O)C(N[C@@H](Cc3cnccc3)C(N(C)C C(N)═O)═O)═O)═O)═O)═O)═O)N3) cc2)═O)═O)NC(C)═O)C3═O)═O)═O)═ O)NC1═O)O |
| | MeCO-Pen(3)-E(2)-T-7MeW-K(Ac)-Pen(3)- | |
| | AEF(2)-2Nal-THP-K(Ac)-N-3Pya-Sar-CONH2 | |
| 189 | 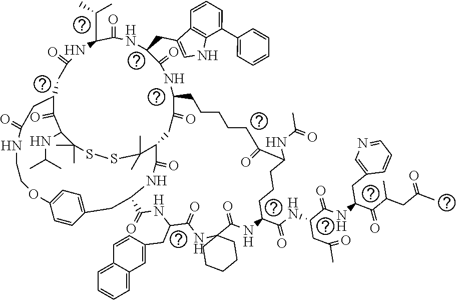 | C[C@H]([C@@H](C(N[C@@H](Cc1c [nHc2c1cccc2═ c1ccccc1)C(N[C@@H](CCCCNC(C)═ O)C(N[C@@H](C(C)(C)SSC(C)(C)[C @@H](C(N[C@H]1CCC(NCCOc2ccc( C[C@@H](C(N[C@@H](Cc3cc4ccccc 4cc3)C(NC3(CCOCC3)C(N[C@@H](C) C(N[C@@H](Cc3cnccc3)C(N(C)CC (N)═O)═O)═O)═O)═O)═O)═O)N3)cc2)═ O)═O)NC(C)═O)C3═O)═O)═O)═O)NC1═ O)O |
| | MeCO-Pen(3)-E(2)-T-7PhW-K(Ac)-Pen(3)- | |
| | AEF(2)-2Nal-THP-K(Ac)-N-3Pya-Sar-CONH2 | |
| 190 | 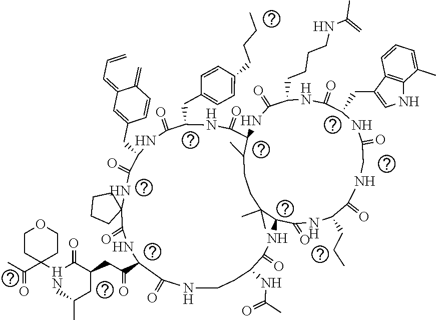 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCC/C═C\CCC[C@@H] (C(N[C@@H](CC(N)═O)C(NC2(CCO CC2)C(N)═O)═O)═O)NC(C2(CCCC2)N C([C@H](Cc2cc3ccccc3cc2)NC([C@H] (Cc(cc2)ccc2OCCN)N2)═O)═O)═O)NC (C)═O)═O)C2═O)═O)═O)═O)NC1═O)O |
| | MeCO-R5H(4)-Pen(3)-N-T-7MeW-K(Ac)-Pen(3)- | |
| | AEF-2Nal-Acpx-S5H(4)-N-THP-CONH2 | |
| 191 | 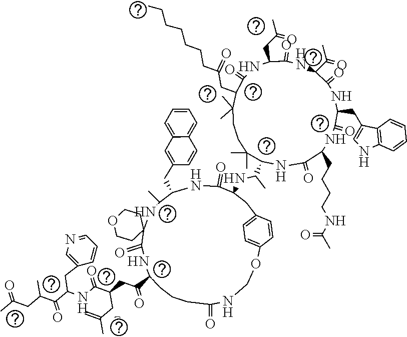 [SMILES: C=C(C)C[C@H](CC(=O)[C@@H]1CCCC(=O)NCOc2ccc(cc2)C[C@H](NC(C)[C@H]2NC(=O)[C@H](CCCCNC(C)=O)NC(=O)[C@H](Cc3c[nH]c4ccccc34)NC(=O)[C@H](C(C)=O)NC(=O)[C@H](CC(C)=O)NC(=O)C(CC(=O)CCCCCCC)C(C)(C)CCC2(C)C)C(=O)N[C@@H](Cc2ccc3ccccc3c2)C(C)NC2(CCOCC2)C(=O)N1)C(=O)NC(Cc1cccnc1)C(=O)C(C)CC(C)=O] | C[C@H]([C@@H](C(N[C@@H](Cc1c [nH]c2c1cccc2)C(N[C@@H](CCCCNC( C)═O)C(N[C@@H](C(C)(C)SSC(C)(C) C(CCCCCCNC(CC[C@@H](C(N[C@ @H](CC(N)═O)C(N[C@@H](Cc2cnccc 2)C(N(C)CC(N)═O)═O)═O)═O)NC(C2 (CCOCC2)NC([C@H](Cc2cc3ccccc3cc2) NC([C@H](Cc(cc2)ccc2OCCN)N2)═ O)═O)═O)═O)═O)C2═O)═O)═O)═O) NC1═O)O |
| | 7Ahp-Pen(3)-N-T-W-K(Ac)-Pen(3)-AEF(2)-2Nal- | |
| | THP-E(2)-N-3Pya-Sar-CONH2 | |
| 192 | 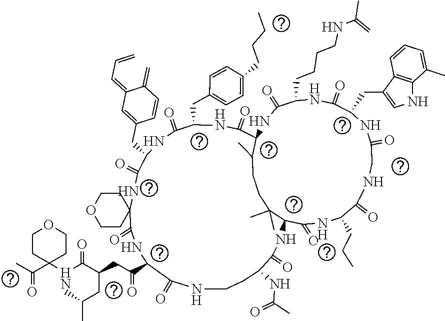 | C[C@H]([C@@H](C(N[C@@H](Cc1c [nH]c2c1cccc2C)C(N[C@@H](CCCCN C(C)═O)C(N[C@@H](C(C)(C)SSC(C) (NC(CCCCCNC(CC[C@@H](C(N[C@ @H](CC(N)═O)C(NC2(CCOCC2)C (N)═O)═O)═O)NC(C2(CCOCC2)NC([C@ H](Cc2cc3ccccc3cc2)NC([C@H] (Cc(cc 2)ccc2OCCN)N2)═O)═O)═O)═O)═O)C 2═O)═O)═O)═O)NC1═O)O |
| | 6Ahx(2)-Pen(3)-N-T-7MeW-K(Ac)-Pen(3)-AEF- | |
| | 2Nal-THP-E(2)-N-THP-CONH2 | |
| 193 | 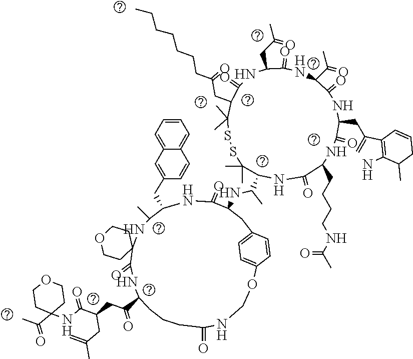 [SMILES: C=C(C)C[C@H](CC(=O)[C@@H]1CCCC(=O)NCOc2ccc(cc2)C[C@H](NC(C)[C@H]2NC(=O)[C@H](CCCCNC(C)=O)NC(=O)[C@H](CC(=C)C3=C(N)C(C)CC=C3)NC(=O)[C@H](C(C)=O)NC(=O)[C@H](CC(C)=O)NC(=O)C(CC(=O)CCCCCCC)C(C)(C)SSC2(C)C)C(=O)N[C@@H](Cc2ccc3ccccc3c2)C(C)NC2(CCOCC2)C(=O)N1)C(=O)NC1(C(C)=O)CCOCC1] | C[C@H]([C@@H](C(N[C@@H](Cc1c [nH]c2c1cccc2C)C(N[C@@H](CCCCN C(C)═O)C(N[C@@H](C(C)(C)SSC(C) C)[C@@H](C(N[C@H]1CC(N)═O)═O) NC(CCCCCN)═O)C(N[C@@H](Cc(cc2) ccc2OCCNC(CC[C@@H](C(N[C@@ H](CC(N)═O)C(NC2(CCOCC2)C(N)═ O)═O)═O)NC(C2(CCOCC2)NC([C@H] (Cc2cc3ccccc3cc2)N2)═O)═O)═O)C2═ O)═O)═O)═O)═O)NC1═O)O |
| | 6Ahx-Pen(3)-N-T-7MeW-K(Ac)-Pen(3)-AEF(2)- | |
| | 2Nal-THP-E(2)-N-THP-CONH2 | |
| 194 | 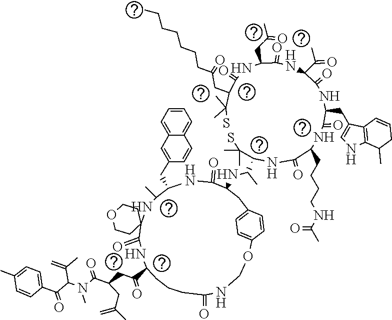 [SMILES: C=C(C)C[C@H](CC(=O)[C@@H]1CCCC(=O)NCOc2ccc(cc2)C[C@H](NC(C)[C@H]2NC(=O)[C@H](CCCCNC(C)=O)NC(=O)[C@H](Cc3c[nH]c4c3C=CCC4C)NC(=O)[C@H](C(C)=O)NC(=O)[C@H](CC(C)=O)NC(=O)C(CC(=O)CCCCCCC)C(C)(C)SSC2(C)C)C(=O)N[C@@H](Cc2ccc3ccccc3c2)C(C)NC2(CCOCC2)C(=O)N1)C(=O)N(C)C(C(=C)C)C(=O)c1ccc(C)cc1] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC(CCCCCCN)═O)C(N[C@@H](Cc(c c2)ccc2OCCNC(CC[C@@H](C(N[C@ @H](CC(N)═O)C(N(C)[C@H](Cc(cc2) ccc2O)C(N)═O)═O)═O)NC(C2(CCOCC 2)NC([C@H](Cc2cc3ccccc3cc2)N2)═ O)═O)═O)C2═O)═O)═O)═O)═O)NC1═ O)O |
| | 7Ahp-Pen(3)-N-T-7MeW-K(Ac)-Pen(3)-AEF(2)- | |
| | 2Nal-THP-E(2)-N-NMeDTyr-CONH2 | |
| 195 | 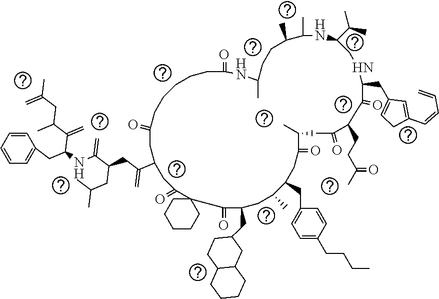 | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCCNC(CC [C@@H](C(N[C@@H](CC(N)═O)C(N [C@@H](Cc2cnccc2)C(N(C)CC(N)═ O)═O)═O)═O)NC(C2(CCOCC2)NC([C@ H](Cc2cc3ccccc3cc2)NC([C@H](Cc(cc 2)ccc2OCCN)N2)═O)═O)═O)═O)═O)C 2═O)═O)═O)═O)NC1═O)O |
| | 6Ahx(2)-Abu(1)-N-T-W-Q-C(1)-AEF-2Nal-THP- | |
| | E(2)-N-3Pya-Sar-CONH2 | |
| 196 | 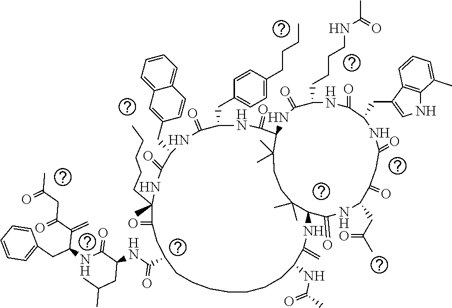 | C[C@H]([C@@H](C(N[C@@H](Cc1c[nH] c2c1cccc2C)C(N[C@@H](CCCCN C(C)═O)C(N[C@@H](C(C)(C)SSC(C)(C) [C@@H](C(N[C@H]1CC(N)═O)═O) NC([C@@H](CCCC/C═C\CCC[C@@ H](C(N[C@@H](CC(N)═O)C(N[C@@ H](Cc2cnccc2)C(N(C)CC(N)═O)═O)═ O)═O)NC([C@](C)(CCCCN)NC([C@H] (Cc2cc3ccccc3cc2)NC([C@H](Cc(cc2)cc c2OCCN)N2)═O)═O)═O)NC(C)═O)═O) C2═O)═O)═O)═O)NC1═O)O |
| | MeCO-R6H(4)-Pen(3)-N-T-7MeW-K(Ac)-Pen(3)- | |
| | AEF-2Nal-aMeK-S5H(4)-N-3Pya-Sar-CONH2 | |
| 197 | 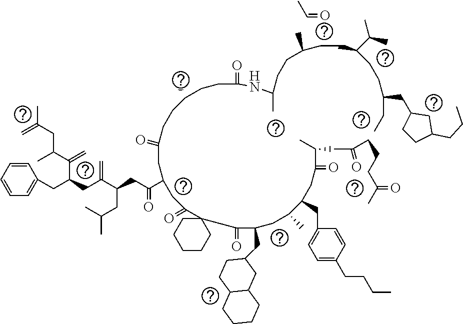 | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCCNC(CC [C@@H](C(N[C@@H](CC(N)═O)C(N [C@@H](Cc2cnccc2)C(N(C)CC(N)═ O)═O)═O)═O)NC([C@](C)(CCCCN)NC ([C@H](Cc2cc3ccccc3cc2)NC([C@H](C c(cc2)ccc2OCCN)N2)═O)═O)═O)═O)═ O)C2═O)═O)═O)═O)NC1═O)O |
| | 6Ahx(2)-Abu(1)-N-T-W-Q-C(1)-AEF-2Nal-aMeK- | |
| | E(2)-N-3Pya-Sar-CONH2 | |
| 198 | 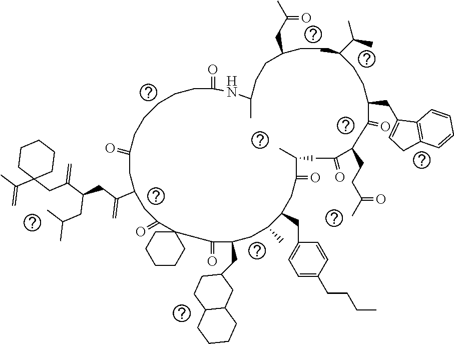 | C[C@H]([C@@H](C(N[C@@H](Cc1c[ nH]c2c1cccc2)C(N[C@@H](CCC(N)═ O)C(N[C@@H](CSCC[C@@H](C(N[C @H]1CC(N)═O)═O)NC(CCCCNC(CC[ C@@H](C(N[C@@H](CC(N)═O)C(N C2(CCOCC2)C(N)═O)═O)═O)NC(C2(C COCC2)NC([C@H](Cc2cc3ccccc3cc2) NC([C@H](Cc(cc2)ccc2OCCN)N2)═ O)═O)═O)═O)═O)C2═O)═O)═O)═O) NC1═O)O |
| | 5Ava(2)-Abu(1)-N-T-W-Q-C(1)-AEF-2Nal-THP- | |
| | E(2)-N-THP-CONH2 | |
| 199 | 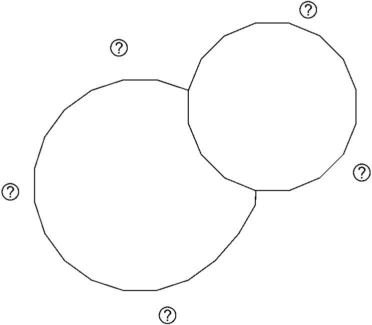 [SMILES: C1CCCCCCCCC2CCCCCCCCCCCC(CCCCCCC1)CCC2] | C[C@H]([C@@H](C(N[C@@H](Cc1c[nH] c2c1cccc2C)C(N[C@@H](CCCCN C(C)═O)C(N[C@@H](C(C)(C)SSC(C)(C) [C@@H](C(N[C@H]1CC(N)═O)═O) NC(CCCCCCNC(CC[C@@H](C(N[C @@H](CC(N)═O)C(NC2(CCOCC2)C (N)═O)═O)═O)NC(C2(CCOCC2)NC([C @H](Cc2cc3ccccc3cc2)NC([C@H](Cc (cc2)ccc2OCCN)N2)═O)═O)═O)═O)═O) C2═O)═O)═O)═O)NC1═O)O |
| | 7Ahp(2)-Pen(3)-N-T-7MeW-K(Ac)-Pen(3)-AEF- | |
| | 2Nal-THP-E(2)-N-THP-CONH2 | |
| 200 | 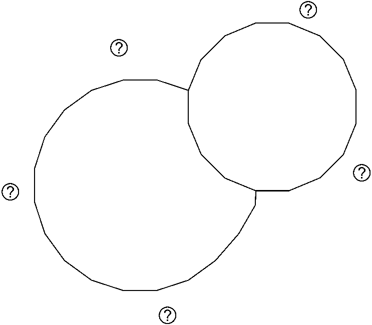 [SMILES: C1CCCCCCCCC2CCCCCCCCCCCC(CCCCCCC1)CCC2] | C[C@H]([C@@H](C(N[C@@H](Cc1c[nH] c2c1cccc2C)C(N[C@@H](CCCCN C(C)═O)C(N[C@@H](C(C)(C)SSC(C)(C) [C@@H](C(N[C@H]1CC(N)═O)═O) NC([C@@H](CCCNC(N)═)NC(CCC NC(CC[C@@H](C(N[C@@H](CC(N)═ O)C(N[C@@H](Cc2cnccc2)C(N(C)CC (N)═O)═O)═O)═O)NC(C2(CCOCC2)NC ([C@H](Cc2cc3ccccc3cc2)NC([C@H] (Cc(cc2)ccc2OCCN)N2)═O)═O)═O)═ O)═O)═O)C2═O)═O)═O)═O)NC1═O)O |
| | GABA(2)-r-Pen(3)-N-T-7MeW-K(Ac)-Pen(3)- | |
| | AEF-2Nal-THP-E(2)-N-3Pya-Sar-CONH2 | |
| 201 | 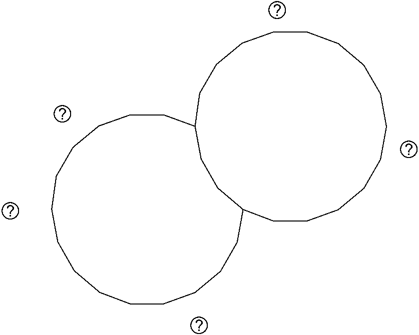 [SMILES: C1CCCCCCCC2CCCCCCCCCCCCCCC(CCCCCC1)CC2] | CC(C)([C@@H](C(N[C@@H](CC(N)═ O)C(N(CC[C@H]1O[C@@H]1C(N[C @@H](Cc1c[nH]c2c1cccc2C)C(N[C@ H]1CCC/C═C\CCC[C@@H](C(N[C@ @H](CCCCNC(C)═O)C(N[C@@H](C C(N)═O)C(N[C@@H](Cc2cnccc2)C(N)═ O)═O)═O)═O)NC([C@H](Cc2cc3cccc c3cc2)NC([C@H](Cc(cc2)ccc2OCCN)N) SSC(C)(C)[C@@H]2NC1═O |
| | MeCO-Pen(3)-N-3OHPro-7MeW-S5H(4)-Pen(3)- | |
| | AEF-2Nal-S5H(4)-K(Ac)-N-3Pya-CONH2 | |
| 202 | 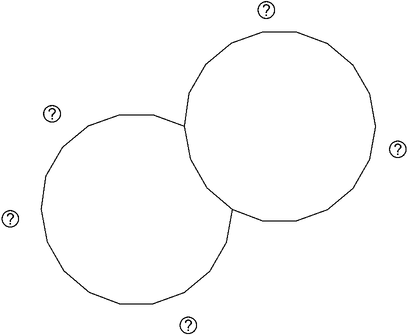 [SMILES: C1CCCCCCCC2CCCCCCCCCCCCCCC(CCCCCC1)CC2] | CC(C)([C@@H](C(N[C@@H](CC(N)═ O)C(N(CC[C@@H]1O[C@@H]1C(N[ C@@H](Cc1c[nH]c2c1cccc2C)C(N[C @H]1CCC/C═C\CCC[C@@H](C(N[C @@H](CCCCNC(C)═O)C(N[C@@H] (CC(N)═O)C(N[C@@H](Cc2cnccc2)C (N)═O)═O)═O)═O)NC([C@H](Cc2cc3cc ccc3cc2)NC([C@H](Cc(cc2)ccc2OCCN) NC2═O)═O)═O)═O)═O)═O)═O)NC (C)═O)SSC(C)(C)[C@@H]2NC1═O |
| | MeCO-Pen(3)-N-3OHPro-7MeW-S5H(4)-Pen(3)- | |
| | AEF-2Nal-S5H(4)-K(Ac)-N-3Pya-CONH2 | |
| 203 | 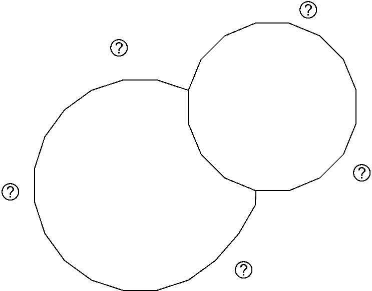 [SMILES: C1CCCCCCCCC2CCCCCCCCCCCC(CCCCCCC1)CCC2] | C[C@H]([C@@H](C(N[C@@H](Cc1c[nH] c2c1cccc2C)C(N[C@@H](CCCCN C(C)═O)C(N[C@@H](C(C)(C)SSC(C)(C) [C@@H](C(N[C@H]1CC(N)═O)═O) NC([C@@H](CCC/C═C\CCC[C@@H] (C(N[C@@H](CC(N)═O)C(N[C@@H] (Cc2cnccc2)C(N(C)CC(N)═O)═O)═O)═ O)NC([C@](C)(CCCCN)NC([C@H](C c2cc3ccccc3cc2)NC([C@H](Cc(cc2)ccc 2OCCN)N2)═O)═O)═O)NC(C)═O)═O) C2═O)═O)═O)═O)NC1═O)O |
| | MeCO-R5H(4)-Pen(3)-N-T-7MeW-K(Ac)-Pen(3)- | |
| | AEF-2Nal-aMeK-S5H(4)-N-3Pya-Sar-CONH2 | |
| 204 | 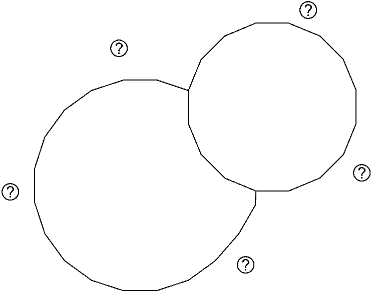 [SMILES: C1CCCCCCCCC2CCCCCCCCCCCC(CCCCCCC1)CCC2] | C[C@H][C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCC/C═C\CCC[C@@H] (C(N[C@@H](CC(N)═O)C(N[C@@H] (Cc2cnccc2)C(N(C)CC(N)═O)═O)═O)═ O)NC([C@](C)(CCCCN)NC([C@H](C c2cc3ccccc3cc2)NC([C@H](Cc(cc2)ccc 2OCCN)N2)═O)═O)═O)NC(C)═O)═O) C2═O)═O)═O)═O)NC1═O)O |
| | MeCO-R5H(4)-Pen(3)-N-T-7MeW-K(Ac)-Pen(3)- | |
| | AEF-2Nal-aMeK-S5H(4)-N-3Pya-Sar-CONH2 | |
| 205 | 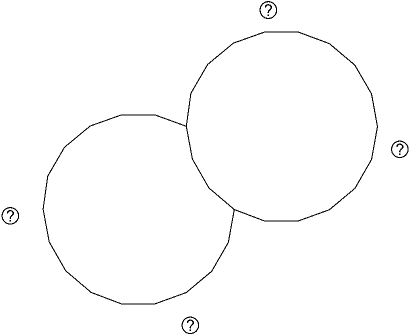 [SMILES: C1CCCCCCCC2CCCCCCCCCCCCCCC(CCCCCC1)CC2] | C[C@H][C@@H](C(N[C@@H](Cc1c[nH] c2c1cccc2C)C(N[C@@H](CCC/C═C\ CCC[C@@H](C(N[C@@H](CCCCNC (C)═O)C(N[C@@H](CC(N)═O)C(N[C @@H](Cc1cnccc1)C(N(C)CC(N)═O)═ O)═O)═O)═O)NC([C@H](Cc1cc2ccccc 2cc1)NC([C@H](Cc(cc1)ccc1OCCN)N C([C@H](C(C)(C)SSC(C)(C)[C@@H] (C(N[C@@H]1CC(N)═O)═O)NC(C)═O) N2)═O)═O)═O)C2═O)═O)═O)NC1═O) O |
| | MeCO-Pen(3)-N-T-7MeW-S5H(4)-Pen(3)-AEF- | |
| | 2Nal-S5H(4)-K(Ac)-N-3Pya-Sar-CONH2 | |
| 206 | 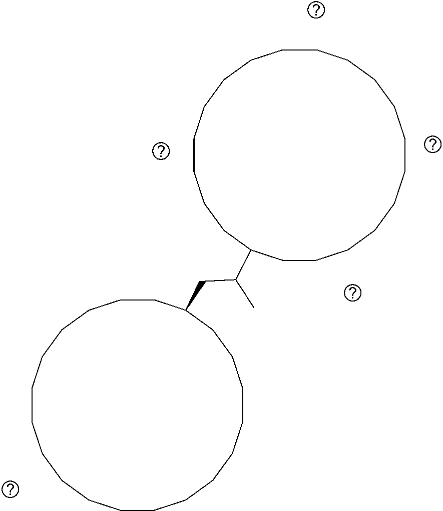 | C[C@H][C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCNC(N)═N)NC(CCC N)═O)═O)C(N[C@@H](Cc(cc2)ccc2O CCNC(CC[C@@H](C(N[C@@H](CC (N)═O)C(N[C@@H](Cc2cnccc2)C(N(C) CC(N)═O)═O)═O)═O)NC(C2(CCOCC2) NC([C@H](Cc2cc3ccccc3cc2)N2)═O)═ O)═O)C2═O)═O)═O)═O)═O)NC1═O)O |
| | GABA-r-Pen(3)-N-T-7MeW-K(Ac)-Pen(3)- | |
| | AEF(2)-2Nal-THP-E(2)-N-3Pya-Sar-CONH2 | |
| 207 | 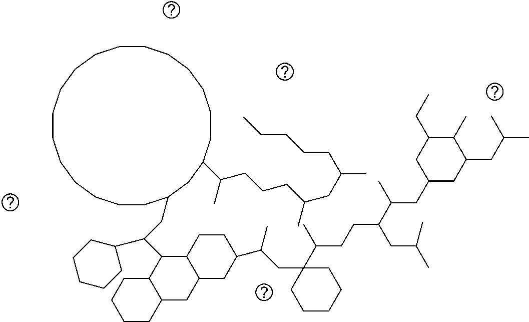 [SMILES: CCCCCC(C)CC(C)CCCC(C)C1CCCCCCCCCCCCCCCCCC(CC(C2CCCCC2)C2C3CCCCC3CC3CC(C(C)CC4(C(C)CCC(CC(C)C)C(C)CC5CC(CC)C(C)C(CC(C)C)C5)CCCCC4)CCC32)C1] | C[C@H][C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCCCNC(C)═O)C(N[C@@H](CC(N)═ O)C(N[C@@H](Cc3cnccc3)C(N(C)C C(N)═O)═O)═O)═O)═O)═O)═O)N3) cc2)═O)═O)NC([C@@H](CCCNC(N)═ N)NC(C)═O)═O)C3═O)═O)═O)═O) NC1═O)O |
| | MeCO-r-Pen(3)-E(2)-T-7MeW-K(Ac)-Pen(3)- | |
| | AEF(2)-2Nal-THP-K(Ac)-N-3Pya-Sar-CONH2 | |
| 208 | 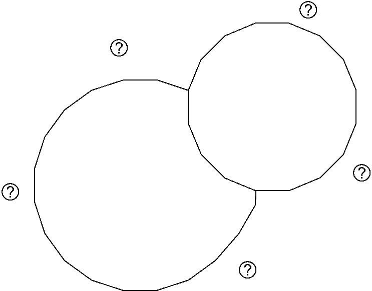 [SMILES: C1CCCCCCCCC2CCCCCCCCCCCC(CCCCCCC1)CCC2] | C[C@H]([C@@H](C(N[C@@H](Cc1c [nHc2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCCC/C═C\CCC[C@ @H](C(N[C@@H](CC(N)═O)C(N[C@ @H](Cc2cnccc2)C(N(C)CC(N)═O)═ O)═O)═O)NC(C2(CCOCC2)NC([C@H](C c2cc3ccccc3cc2)NC([C@H](Cc(cc2)ccc 2OCCN)N2)═O)═O)═O)NC(C)═O)═O) C2═O)═O)═O)═O)NC1═O)O |
| | MeCO-R7H(4)-Pen(3)-N-T-7MeW-K(Ac)-Pen(3)- | |
| | AEF-2Nal-THP-S5H(4)-N-3Pya-Sar-CONH2 | |
| 209 | 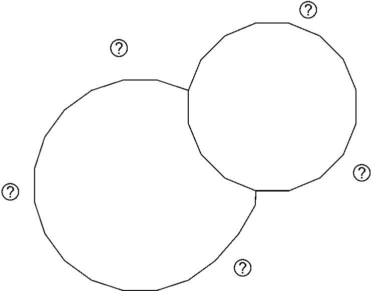 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCCC/C═C\CCC[C@ @H](C(N[C@@H](CC(N)═O)C(N[C@ @H](Cc2cnccc2)C(N(C)CC(N)═O)═ O)═O)═O)NC(C2(CCOCC2)NC([C@H](C c2cc3ccccc3cc2)NC([C@H](Cc(cc2)ccc 2OCCN)N2)═O)═O)═O)NC(C)═O)═O) C2═O)═O)═O)═O)NC1═O)O |
| | MeCO-R7H(4)-Pen(3)-N-T-7MeW-K(Ac)-Pen(3)- | |
| | AEF-2Nal-THP-S5H(4)-N-3Pya-Sar-CONH2 | |
| 210 | 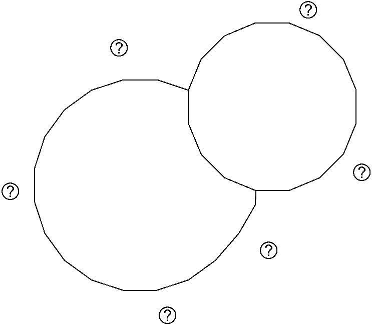 [SMILES: C1CCCCCCCCC2CCCCCCCCCCCC(CCCCCCC1)CCC2] | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC([C@@H](CCN C(CSC[C@@H](C(N[C@@H](CC(N)═ O)C(N[C@@H](Cc2cnccc2)C(N(C)CC (N)═O)═O)═O)═O)NC(C2(CCOCC2)NC ([C@H](Cc2cc3ccccc3cc2)NC([C@H] (Cc(cc2)ccc2OCCN)N2)═O)═O)═O)═O) NC(C)═O)═O)C2═O)═O)═O)═O)NC1═ O)O |
| | MeCO-dab(COCH2(1*))-Abu(1)-N-T-W-Q-C(1)- | |
| | AEF-2Nal-THP-C(1*)-N-3Pya-Sar-CONH2 | |
| 211 | 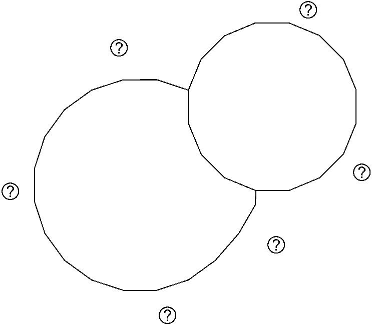 [SMILES: C1CCCCCCCCC2CCCCCCCCCCCC(CCCCCCC1)CCC2] | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCNC(CC [C@@H](C(N[C@@H](CC(N)═O)C(N [C@@H](Cc2cnccc2)C(N(C)CC(N)═O)═ O)═O)═O)NC(C2(CCOCC2)NC([C@ H](Cc2cc3ccccc3cc2)NC([C@H](Cc(cc 2)ccc2OCCN)N2)═O)═O)═O)═O)═O)C 2═O)═O)═O)═O)NC1═O)O |
| | 5Ava(2)-Abu(1)-N-T-W-Q-C(1)-AEF-2Nal-THP- | |
| | E(2)-N-3Pya-Sar-CONH2 | |
| 212 | 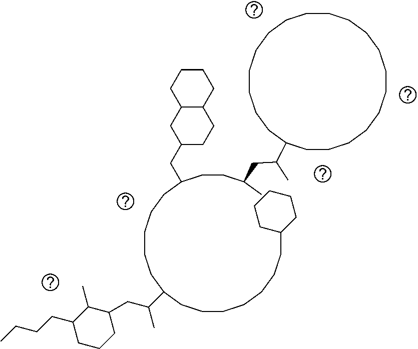 [SMILES: CCCCC1CCCC(CC(C)C(CCCCCCCC2CCCCC2)CCCCCC(CC[C@H](C)CC(C)C2CCCCCCCCCCCCCCCCCCC2)CC2CCC3CCCCC3C2)C1C] | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCN)═O)C (N[C@@H](Cc(cc2)ccc2OCCNC(CC[C @@H](C(N[CAAH](CC(N)═O)C(N[C @@H](Cc2cnccc2)C(N(C)CC(N)═O)═ O)═O)═O)NC(C2(CCOCC2)NC([C@H] (Cc2cc3ccccc3cc2)N2)═O)═O)═O)C2═ O)═O)═O)═O)═O)NC1═O)O |
| | 5Ava-Abu(1)-N-T-W-Q-C(1)-AEF(2)-2Nal-THP- | |
| | E(2)-N-3Pya-Sar-CONH2 | |
| 213 | 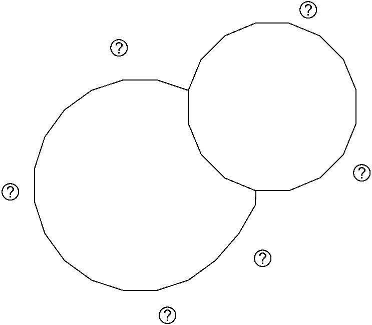 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN C(C)═O)C(N[C@@H](CSCC[C@@H] (C(N[C@H]1CC(N)═O)═O)NC(CCCCC NC(CC[C@@H](C(N[C@@H](CC(N)═ O)C(N[C@@H](Cc2cncn2C)C(N(C)CC (N)═O)═O)═O)═O)NC(C2(CCOCC2)N C([C@H](Cc2cc3ccccc3cc2)NC([C@H] (Cc(cc2)ccc2OCCN)N2)═O)═O)═O)═ O)═O)C2═O)═O)═O)═O)NC1═O)O |
| | 6Ahx(2)-Abu(1)-N-T-7MeW-K(Ac)-C(1)-AEF- | |
| | 2Nal-THP-E(2)-N-3MeH-Sar-CONH2 | |
| 214 | 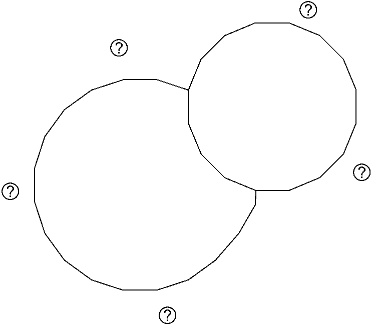 [SMILES: C1CCCCCCCCC2CCCCCCCCCCCC(CCCCCCC1)CCC2] | C[C@H]([C@@H](C(N[C@@H](Cc1c [nH]c2c1cccc2)C(N[C@@H](CCC(N)═ O)C(N[C@@H](C(C)(C)SSC(C)(C)[C @@H](C(N[C@H]1CCC(N)═O)═O)NC ([C@@H](CCCCNC(CC[C@@H](C(N [C@@H](CC(N)═O)C(N[C@@H](Cc2 cnccc2)C(N(C)CC(N)═O)═O)═O)═O)N C(C2(CCOCC2)NC([C@H](Cc2cc3cccc c3cc2)NC([C@H](Cc(cc2)ccc2OCCN)N 2)═O)═O)═O)═O)NC(C)═O)═O)C2═ O)═O)═O)═O)NC1═O)O |
| | MeCO-lys(2)-Pen(3)-Q-T-W-Q-Pen(3)-AEF-2Nal- | |
| | THP-E(2)-N-3Pya-Sar-CONH2 | |
| 215 | 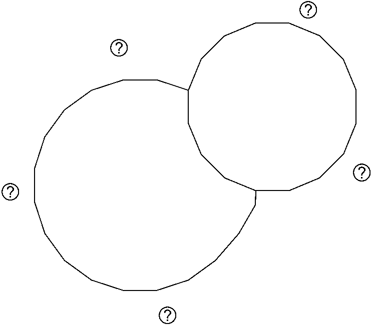 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCCCNC(CO CCOCCNC(COCCOCCNC(CC[C@@H] (C(O)═O)NC(CCCCCCCCCCCCCCC CC(O)═O)═O)═O)═O)═O)═O)NC(CCC CCCNC(CC[C@@H](C(N[C@@H](C C(N)═O)C(N[C@@H](Cc2cnccc2)C(N (C)CC(N)═O)═O)═O)═O)NC(C2(CCOC C2)NC([C@H](Cc2cc3ccccc3cc2)NC ([C@H](Cc(cc2)ccc2OCCN)N2)═O)═O)═ O)═O)═O)C2═O)═O)═O)═O)NC1═O)O |
| | MeCO-R5H(4)-Pen(3)-Q-T-W-Q-Pen(3)-AEF- | |
| | 2Nal-THP-S5H(4)-N-3Pya-Sar-CONH2 | |
| 216 | 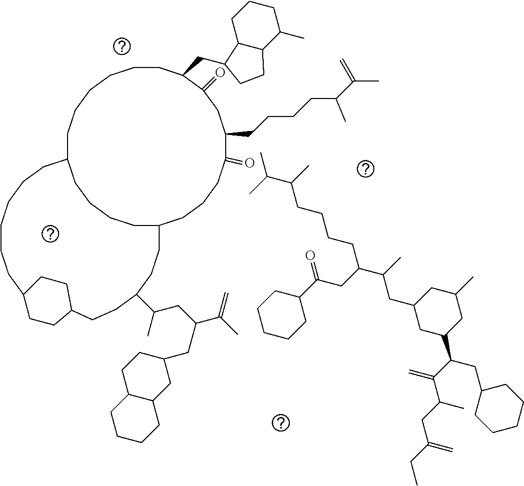 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCCNC(COCCOCCNC (COCCOCCNC(CC[C@@H](C(O)═O) NC(CCCCCCCCCCCCCCCCC(O)═ O)═O)═O)═O)═O)NC(CCCNC(CC[C@@ H](C(N[C@@H](CC(N)═O)C(N[C@@ H](Cc2cnccc2)C(N(C)CC(N)═O)═O)═ O)═O)NC(C2(CCOCC2)NC([C@H](Cc2c c3ccccc3cc2)NC([C@H](Cc(cc2)ccc2O CCN)N2)═O)═O)═O)═O)═O)═O)C2═ O)═O)═O)═O)NC1═O)O |
| | MeCO-Pen(3)-E(2)-T-7MeW-K(NMeAc)-Pen(3)- | |
| | AEF(2)-2Nal-THP-K(NMeAc)-N-3Pya-Sar- | |
| | CONHMe | |
| 217 | 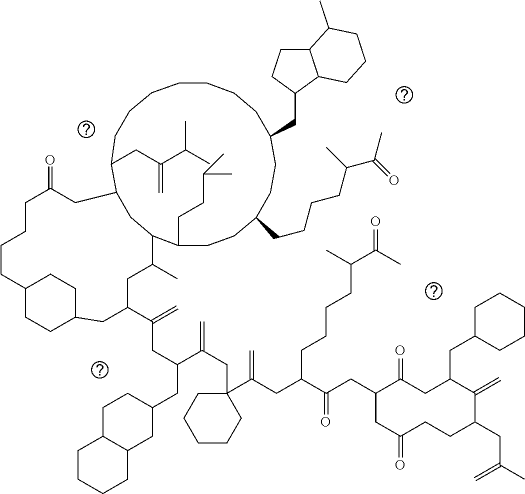 [SMILES: C=C(C)CC1CCC(=O)CC(CC(=O)C(CCCCC(C)C(C)=O)CC(=C)C2(CC(=C)C(CC(=C)C3CC4CCC(CCCCC(=O)CC5CC(C(C)C3)C(CCC(C)C)CC[C@H](CCCCC(C)C(C)=O)CC[C@H](CC3CCC6C(C)CCCC36)CCCCCCCC5CC(=C)C(C)C)CC4)CC3CCC4CCCCC4C3)CCCCC2)C(=O)CC(CC2CCCCC2)C1=C] | C[C@H]([C@@H](C(N[C@@H](Cc1c [nH]c2c1cccc2)C(N[C@@H](CCC(N)═ O)C(N[C@@H](C(C)(C)SSC(C)(C)[C @@H](C(N[C@H]1CCC(N)═O)═O)NC ([C@@H](CCC/C═C\CCC[C@@H](C (N[C@@H](CC(N)═O)C(N[C@@H](Cc 2cnccc2)C(N(C)CC(N)═O)═O)═O)═O) NC(C2(CCOCC2)NC([C@H](Cc2cc3cc ccc3cc2)NC([C@H](Cc(cc2)ccc2OCCN) N2)═O)═O)═O)NC(C)═O)═O)C2═O)═ O)═O)═O)NC1═O)O |
| | MeCO-Pen(3)-E(2)-T-7MeW-K(NMeAc)-Pen(3)- | |
| | AEF(2)-2Nal-THP-K(NMeAc)-N-3Pya-Sar- | |
| | CONH2 | |
| 218 | 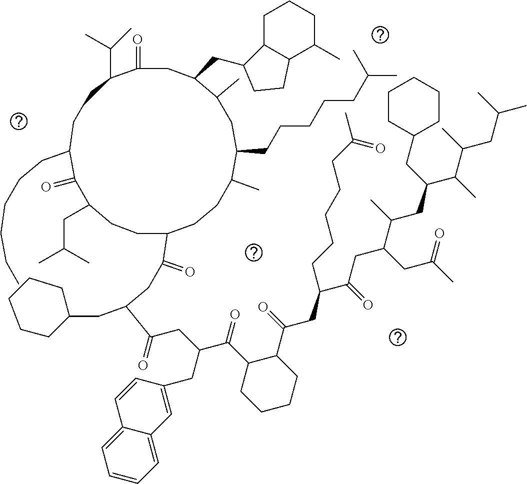 | C[C@H][[C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCCNC(CC [C@@H](C(N[C@@H](CC(N)═O)C(N [C@@H](Cc2cnccc2)C(N(C)[C@@H] (CCCCNC(COCCOCCNC(COCCOCCN C(CC[C@@H](C(O)═O)NC(CCCCCC CCCCCCCCCCC(O)═O)═O)═O)═O)═O) C(N)═O)═O)═O)═O)NC(C2(CCOCC2) NC([C@H](Cc2cc3ccccc3cc2)NC([C@ H](Cc(cc2)ccc2OCCN)N2)═O)═O)═O)═ O)═O)C2═O)═O)═O)═O)NC1═O)O |
| | MeCO-Pen(3)-E(2)-T-7MeW-K(Ac)-Pen(3)- | |
| | AEF(NMe)(2)-2Nal-THP-K(Ac)-N-3Pya-Sar- | |
| | CONH2 | |
| 219 | 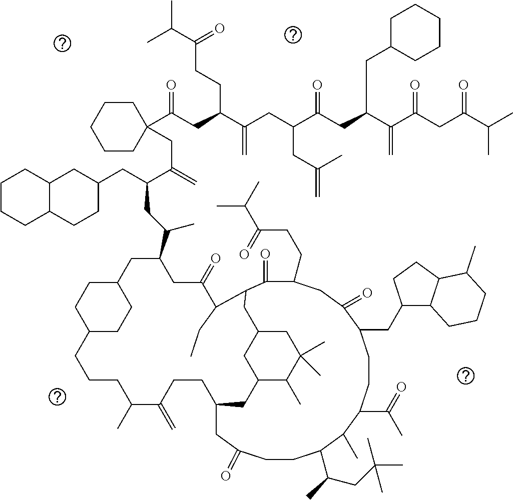 [SMILES: C=C(C)CC(CC(=C)[C@H](CCC(=O)C(C)C)CC(=O)C1(CC(=C)[C@H](CC2CCC3CCCCC3C2)CC(C)[C@H]2CC(=O)C(CC)C3CC4CC(C[C@H](CCC(=C)C(C)CCCC5CCC(CC5)C2)CC(=O)CCC([C@H](C)CC(C)(C)C)C(C)C(C(C)=O)CCC(CC2CCC5C(C)CCCC25)C(=O)CC(CCC(=O)C(C)C)C3=O)C(C)C(C)(C)C4)CCCCC1)C(=O)C[C@@H](CC1CCCCC1)C(=C)C(=O)CC(=O)C(C)C] | C[C@H][[C@@H](C(N[C@@H](Cc1c [nH]c2c1cccc2)C(N[C@@H](CCC(N)═ O)C(N[C@@H](CSCC[C@@H](C(N[C @H]1CCCCNC(COCCOCCNC(COCC OCCNC(CC[C@@H](C(O)═O)NC(CC CCCCCCCCCCCCCCC(O)═O)═O)═O)═ O)═O)═O)NC(CCCCCNC(CC[C@@H] (Cc2cnccc2)C(N(C)CC(N)═O)═O)═ O)═O)NC(C2(CCOCC2)NC([C@H](Cc2c c3ccccc3cc2)NC([C@H](Cc(cc2)ccc2O CCN)N2)═O)═O)═O)═O)═O)C2═O)═ O)═O)═O)NC1═O)O |
| | CF3CO-Pen(3)-E(2)-T-7MeW-Q(N(Me)2)-Pen(3)- | |
| | AEF(NMe)(2)-2Nal-THP-Q(N(Me)2)-N-3Pya-Sar- | |
| | CON(Me)2 | |
| 220 | 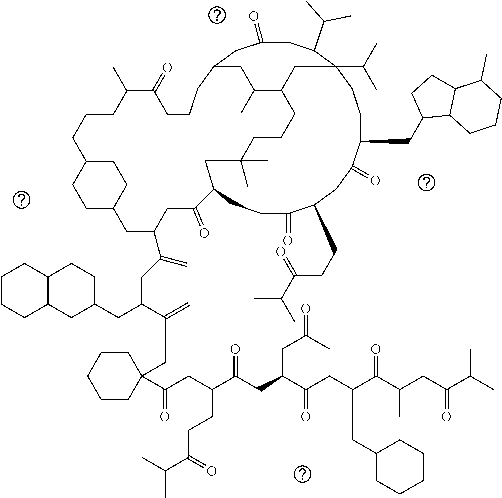 [SMILES: C=C(CC1(C(=O)CC(CCC(=O)C(C)C)C(=O)C[C@@H](CC(C)=O)C(=O)CC(CC2CCCCC2)C(=O)C(C)CC(=O)C(C)C)CCCCC1)C(CC(=C)C1CC(=O)[C@@H]2CCC(=O)[C@H](CCC(=O)C(C)C)CC(=O)[C@H](CC3CCC4C(C)CCCC34)CCC3(C(C)C)CC(CCCC(C)(C)C2)C(C)CC(CCCC(=O)C(C)CCCC2CCC(CC2)C1)CC(=O)CC3C(C)C)CC1CCC2CCCCC2C1] | C[C@H][[C@@H](C(N[C@@H](Cc1c [nH]c2c1cccc2)C(N[C@@H](CCC(N)═ @H]1CCCCNC(COCCOCCNC(COCC OCCNC(CC[C@@H](C(O)═O)NC(CC CCCCCCCCCCCCCCC(O)═O)═O)═O)═ O)═O)═O)NC(CCCCNC(CC[C@@H] (Cc2cnccc2)C(N(C)CC(N)═O)═O)═O)═ O)NC(C2(CCOCC2)NC([C@H](Cc2cc3 ccccc3cc2)NC([C@H](Cc(cc2)ccc2OCC N)N2)═O)═O)═O)═O)═O)C2═O)═O)═ O)═O)NC1═O)O |
| | MeCO-Pen(3)-E(2)-T-7MeW-Q(N(Me)2)-Pen(3)- | |
| | AEF(NMe)(2)-2Nal-THP-Q(N(Me)2)-N-3Pya-Sar- | |
| | CON(Me)2 | |
| 221 | 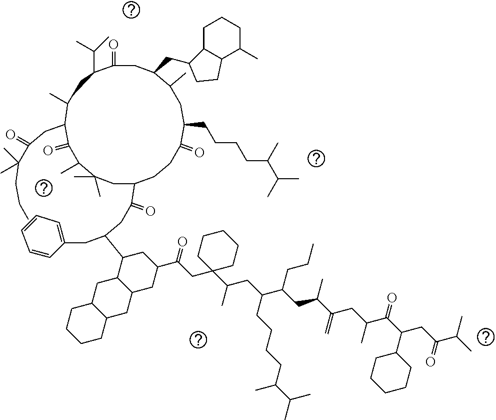 | C[C@H][[C@@H](C(N[C@@H](Cc1c [nH]c2c1cccc2C)C(N[C@@H](CCCCN C(C)═O)C(N[C@@H](C(C)(C)SSC(C) (NC([C@@H](CCCNC(N)═N)NC([C@ @H](CCCNC(CC[C@@H](C(N[C@@ H](CC(N)═O)C(N[C@@H](Cc2cnccc2) C(N(C)CC(N)═O)═O)═O)═O)NC(C2(C COCC2)NC([C@H](Cc2cc3ccccc3cc2) NC([C@H](Cc(cc2)ccc2OCCN)N2)═O)═ O)═O)═O)NC(COCCOCCNC(COCCO CCNC(CC[C@@H](C(O)═O)NC(CCC CCCCCCCCCCCC(O)═O)═O)═O)═O)═ O)═O)═O)C2═O)═O)═O)═O)NC1═O)O |
| | CF3CO-Pen(3)-E(2)-T-7MeW-K(NMeAc)-Pen(3)- | |
| | AEF(2)-2Nal-THP-K(NMeAc)-N-3Pya-Sar- | |
| | CON(Me)2 | |
| 222 | 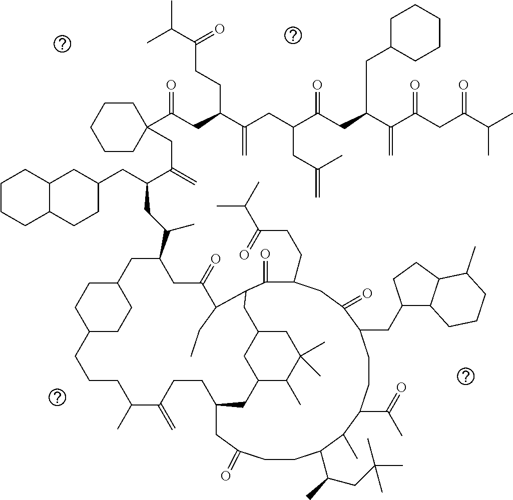 [SMILES: C=C(C)CC(CC(=C)[C@H](CCC(=O)C(C)C)CC(=O)C1(CC(=C)[C@H](CC2CCC3CCCCC3C2)CC(C)[C@H]2CC(=O)C(CC)C3CC4CC(C[C@H](CCC(=C)C(C)CCCC5CCC(CC5)C2)CC(=O)CCC([C@H](C)CC(C)(C)C)C(C)C(C(C)=O)CCC(CC2CCC5C(C)CCCC25)C(=O)CC(CCC(=O)C(C)C)C3=O)C(C)C(C)(C)C4)CCCCC1)C(=O)C[C@@H](CC1CCCCC1)C(=C)C(=O)CC(=O)C(C)C] | C[C@H][[C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CCC(NCC Oc2ccc(C[C@@H](C(N[C@@H](Cc3c c4ccccc4cc3)C(NC3(CCOCC3)C(N[C@ @H](CCCCN(C)C(C)═O)C(N[C@@H] (CC(N)═O)C(N[C@@H](Cc3cnccc3)C (N(C)CC(NC)═O)═O)═O)═O)═O)═O)═ O)N3)cc2)═O)═O)NC(C)═O)C3═O)═ O)═O)═O)NC1═O)O |
| | CF3CO-Pen(3)-E(2)-T-7MeW-Q(N(Me)2)-Pen(3)- | |
| | AEF(2)-2Nal-THP-Q(N(Me)2)-N-3Pya-Sar- | |
| | CON(Me)2 | |
| 223 | 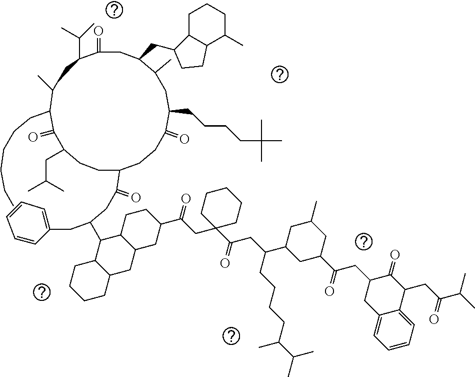 | C[C@H][[C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CCC(NCC Oc2ccc(C[C@@H](C(N[C@@H](Cc3c c4ccccc4cc3)C(NC3(CCOCC3)C(N[C@ (CC(N)═O)C(N[C@@H](Cc3cnccc3)C (N(C)CC(N)═O)═O)═O)═O)═O)═O)═O) N3)cc2)═O)═O)NC(C)═O)C3═O)═O)═ O)═O)NC1═O)O |
| | CF3CO-Pen(3)-E(2)-T-7MeW-K(Me)3-Pen(3)- | |
| | AEF(2)-2Nal-THP-K(NMeAc)-N-3Pya-Sar- | |
| | CON(Me)2 | |
| 224 | 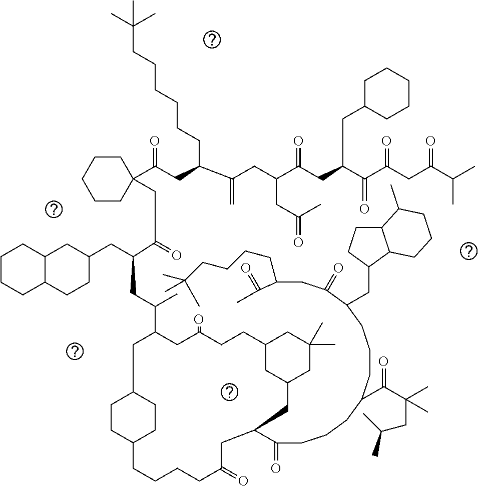 | C[C@H][[C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(N(C)CC Oc2ccc(C[C@@H](C(N[C@@H](Cc3c c4ccccc4cc3)C(NC3(CCOCC3)C(N[C@ @H](CCCCNC(C)═O)C(N[C@@H](C C(N)═O)C(N[C@@H](Cc3cnccc3)C(N (C)CC(N)═O)═O)═O)═O)═O)═O)═O) N3)cc2)═O)═O)NC(C)═O)C3═O)═ O)═O)═O)NC1═O)O |
| | CF3CO-Pen(3)-E(2)-T-7MeW-K(Me)3-Pen(3)- | |
| | AEF(2)-2Nal-THP-K(Me)3-N-3Pya-Sar- | |
| | CON(Me)2 | |
| 225 | 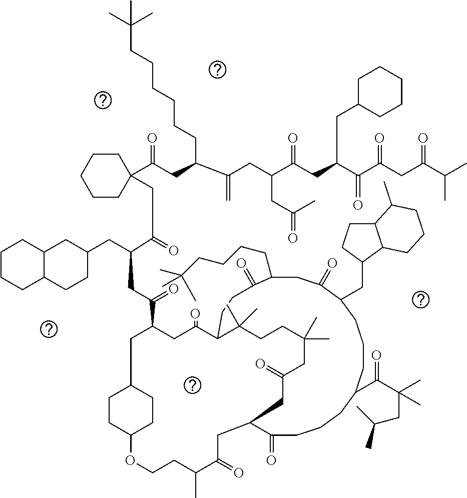 | C[C@H][[C@@H](C(N[C@@H](Cc1c [nH]c2c1cccc2C)C(N[C@@H] (CCC(N (C)C)═O)C(N[C@@H](C(C)(C)SSC(C) (C)[C@@H](C(N[C@H]1CCC(N(C)CC Oc2ccc(C[C@@H](C(N[C@@H](Cc3c c4ccccc4cc3)C(NC3(CCOCC3)C(N[C@ @H](CCC(N(C)C)═O)C(N[C@@H](C C(N)═O)C(N[C@@H](Cc3cnccc3)C(N (C)CC(N(C)C)═O)═O)═O)═O)═O)═O)═ O)N3)cc2)═O)═O)NC(C(F)(F)F)═O) C3═O)═O)═O)═O)NC1═O)O |
| | CF3CO-Pen(3)-E(2)-T-7MeW-K(Me)3-Pen(3)- | |
| | AEF(NMe)(2)-2Nal-THP-K(Me)3-N-3Pya-Sar- | |
| | CON(Me)2 | |
| 226 | 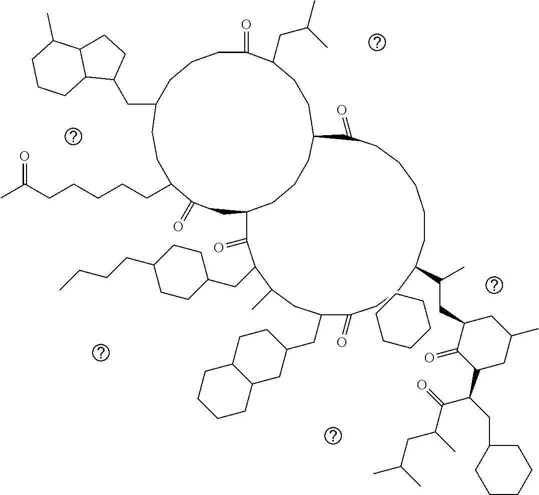 | C[C@H][[C@@H](C(N[C@@H](Cc1c [nH]c2c1cccc2C)C(N[C@@H] (CCC(N (C)C)═O)C(N[C@@H](C(C)(C)SSC(C) (C)[C@@H](C(N[C@H]1CCC(N(C)CC Oc2ccc(C[C@@H](C(N[C@@H](Cc3c c4ccccc4cc3)C(NC3(CCOCC3)C(N[C@ @H](CCC(N(C)C)═O)C(N[C@@H](C C(N)═O)C(N[C@@H](Cc3cnccc3)C(N (C)CC(N(C)C)═O)═O)═O)═O)═O)═ O)═O)N3)cc2)═O)═O)NC(C)═O)C3═ O)═O)═O)═O)NC1═O)O |
| | pFS(6)-Gly-Pen(3)-N-T-7MeW-K(Ac)-Pen(3)- | |
| | AEF-2Nal-THP-Dap(pF(6))-N-3Pya-Sar-CONH2 | |
| 227 | 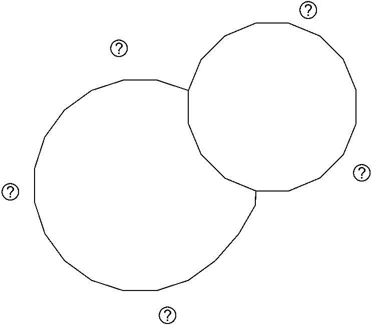 [SMILES: C1CCCCCCCCC2CCCCCCCCCCCC(CCCCCCC1)CCC2] | C[C@H][[C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CCC(NCC Oc2ccc(C[C@@H](C(N[C@@H](Cc3c c4ccccc4cc3)C(NC3(CCOCC3)C(N[C@ @H](CCCCN(C)C(C)═O)C(N[C@@H] (CC(N)═O)C(N[C@@H](Cc3cnccc3)C (N(C)CC(N(C)C)═O)═O)═O)═O)═O)═ O)═O)N3)cc2)═O)═O)NC(C(F)(F)F)═ O)C3═O)═O)═O)═O)NC1═O)O |
| | PEG2(2)-Pen(3)-N-T-7MeW-K(Ac)-Pen(3)-AEF- | |
| | 2Nal-THP-hE(2)-N-3Pya-Sar-CONH2 | |
| 228 | 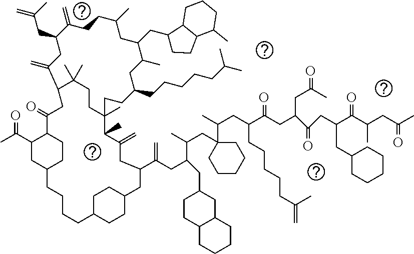 | C[C@H][[C@@H](C(N[C@@H](Cc1c [nH]c2c1cccc2C)C(N[C@@H] (CCC(N (C)C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCC(N(C)C)═O)C(N[C@@H](CC(N)═ O)C(N[C@@H](Cc3cnccc3)C(N(C)C C(N(C)C)═O)═O)═O)═O)═O)═O)═O)N 3)cc2)═O)═O)NC(C(F)(F)F)═O)C3═O)═ O)═O)═O)NC1═O)O |
| | MeCO-GLU(2)-Pen(3)-N-T-7MeW-K(Ac)-Pen(3)- | |
| | AEF(2)-2Nal-THP-K(Ac)-N-3Pya-Sar-CONH2 | |
| 229 | 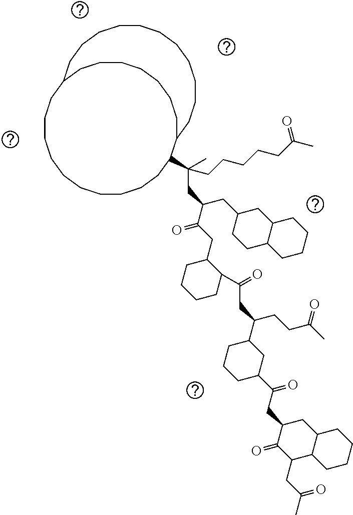 [SMILES: CC(=O)CCCCC[C@@](C)(C[C@@H](CC1CCC2CCCCC2C1)C(=O)CC1CCCCC1C(=O)C[C@@H](CCC(C)=O)C1CCCC(C(=O)C[C@H]2CC3CCCCC3C(CC(C)=O)C2=O)C1)C1CCCCCCCCCCC2CCCCCCCCCCCC1CCCCCCC2] | C[C@H]([C@@H](C(N[C@@H](Cc1c [nH]c2c1cccc2C)C(N[C@@H](CCCC[N+] (C)(C)C)C(N[C@@H](C(C)(C)SSC( C)(C)[C@@H](C(N[C@H]1CCC(NCC Oc2ccc(C[C@@H](C(N[C@@H](Cc3c c4ccccc4cc3)C(NC3(CCOCC3)C(N[C@ @H](CCCCN(C)C(C)═O)C(N[C@@H] (CC(N)═O)C(N[C@@H](Cc3cnccc3)C (N(C)CC(N(C)C)═O)═O)═O)═O)═O)═ O)═O)N3)cc2)═O)═O)NC(C(F)(F)F)═ O)C3═O)═O)═O)═O)NC1═O)O |
| | 5cpaCO-Pen(3)-E(2)-T-7MeW-K(Ac)-Pen(3)- | |
| | AEF(2)-2Nal-THP-E-N-3Pya-Sar-CONH2 | |
| 230 | 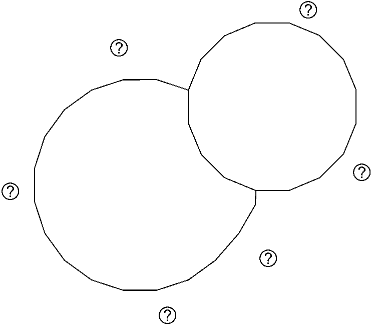 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCC[N+] (C)(C)C)C(N[C@@H](C(C)(C)SSC(C) (C)[C@@H](C(N[C@H]1CCC(NCC Oc2ccc(C[C@@H](C(N[C@@H](Cc3c c4ccccc4cc3)C(NC3(CCOCC3)C(N[C@ CC(N)═O)C(N[C@@H](Cc3cnccc3)C(N (C)CC(N(C)C)═O)═O)═O)═O)═O)═O)═ O)N3)cc2)═O)═O)NC(C(F)(F)F)═O)C 3═O)═O)═O)═O)NC1═O)O |
| | 5cpaCO-k(2)-Pen(3)-N-T-7MeW-K(Ac)-Pen(3)- | |
| | AEF-2Nal-THP-hE(2)-N-3Pya-Sar-CONH2 | |
| 231 | 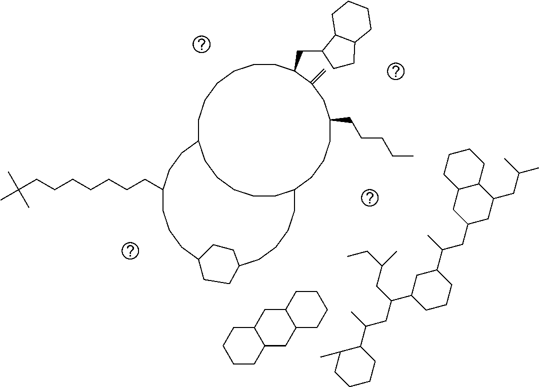 | C[C@H]([C@@H](C(N[C@@H](Cc1c [nH]c2c1cccc2C)C(N[C@@H](CCCC[N+] (C)(C)C)C(N[C@@H](C(C)(C)SSC (C)(C)[C@@H](C(N[C@H]1CCC(N(C) CCOc2ccc(C[C@@H](C(N[C@@H](C c3cc4ccccc4cc3)C(NC3(CCOCC3)C(N [C@@H](CCCC[N+](C)(C)C)C(N[C@ 3)C(N(C)CC(N(C)C)═O)═O)═O)═O)═ O)═O)═O)N3)cc2)═O)═O)NC(C(F)(F) F)═O)C3═O)═O)═O)═O)NC1═O)O |
| | cPEG3aCO-Pen(3)-E(2)-T-7MeW-K(Ac)-Pen(3)- | |
| | AEF(2)-2Nal-THP-K(Ac)-N-3Pya-Sar-CONH2 | |
| 232 | 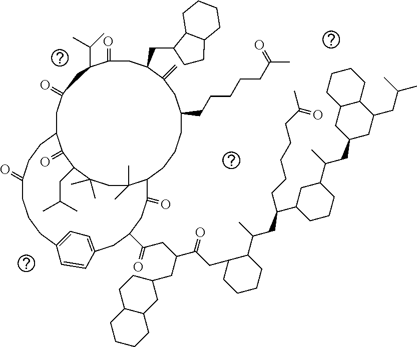 [SMILES: C=C1C[C@@H](CCCCCC(C)=O)CCCC2C(=O)CC(C(=O)CC(CC3CCC4CCCCC4C3)C(=O)CC3CCCCC3C(C)C[C@@H](CCCCCC(C)=O)C3CCCC(C(C)C[C@H]4CC5CCCCC5C(CC(C)C)C4)C3)Cc3ccc(cc3)CCCC(=O)CCC(CC(=O)C[C@@H](C(C)C)C(=O)C[C@H]1CC1CCC3CCCCC31)C(=O)C(CC(C)C)C(C)(C)CC2(C)C] | C[C@H]([C@@H](C(N[C@@H](Cc1c [nH]c2c1cccc2C)C(N[C@@H](CCCCN C(C)═O)C(N[C@@H](C(C)(C)SSC(C) (NC(CNc(c(F)c(c(Sc(c(F)c(c(NC[C@@ H](Cc2cnccc2)C(N(C)CC(N)═O)═O)═ O)═O)NC(C2(CCOCC2)NC([C@H](Cc2c c3ccccc3cc2)NC([C@H](Cc(cc2)ccc2O CCN)N2)═O)═O)═O)c3F)F)c3F)c3F)F)c 3F)═O)C2═O)═O)═O)═O)NC1═O)O |
| | MeCO-Pen(3)-E(2)-T-W-K(Ac)-Pen(3)-AEF-2Nal- | |
| | THP-K(Ac)-S-3Pya-Sar-CONH2 | |
| 233 | 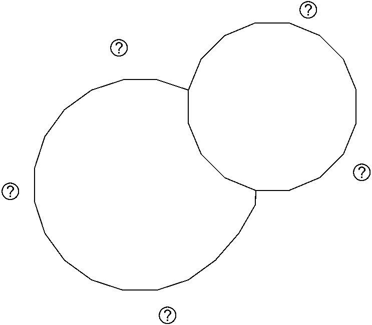 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN C)[C@@H](C(N[C@H]1CC(N)═O)═O) NC(COCCOCCNC(CCC[C@@H](C(N [C@@H](CC(N)═O)C(N[C@@H](Cc2c nccc2)C(N(C)CC(N)═O)═O)═O)═O)NC (C2(CCOCC2)NC([C@H](Cc2cc3ccccc 3cc2)NC([C@H](Cc(cc2)ccc2OCCN)N) NC1═O)O |
| | MeCO-E(2)-Pen(3)-N-T-7MeW-K(Ac)-Pen(3)- | |
| | AEF-2Nal-THP-K(Ac)-N-3Pya-dK(2)-CONH2 | |
| 234 | 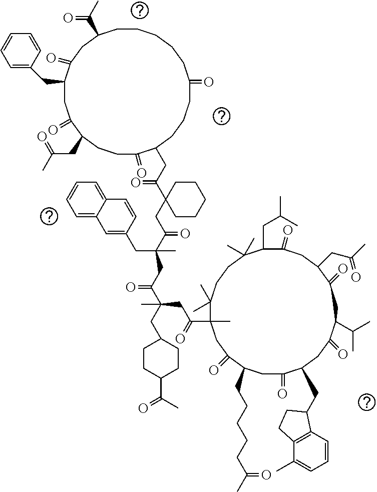 [SMILES: CC(=O)CCCCC[C@@H]1CC(=O)[C@H](CC2CCc3c(C)cccc32)CC(=O)[C@H](C(C)C)CC(=O)C(CC(C)=O)CC(=O)C(CC(C)C)C(C)(C)CCC(C)(C)C(C)(C(=O)C[C@](C)(CC2CCC(C(C)=O)CC2)C(=O)C[C@@](C)(Cc2ccc3ccccc3c2)C(=O)CC2(C(=O)CC3CCCC(=O)CCCCC[C@H](C(C)=O)CC(=O)[C@H](Cc4ccccc4)CC(=O)[C@H](CC(C)=O)CCC3=O)CCCCC2)CC1=O] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@H](CCC(NCCOc2ccc(C[C@@ H](C(N[C@@H](Cc3cc4ccccc4cc3)C(N C3(CCOCC3)C(N[C@@H](CCCCNC (C)═O)C(N[C@@H](CC(N)═O)C(N[C @@H](Cc3cnccc3)C(N(C)CC(N)═O)═ O)═O)═O)═O)═O)═O)N3)cc2)═O)NC (C)═O)═O)C3═O)═O)═O)═O)NC1═O)O |
| | MeCO-Pen(3)-N-T-7MeW-K(Ac)-Pen(3)-4AmF- | |
| | 2Nal-THP-E(2)-N-3Pya-dK(2)-CONH2 | |
| 235 | 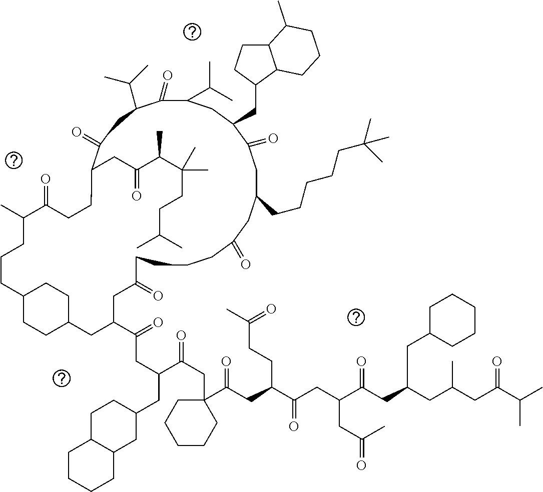 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCC(O)═O)C(N[C@@H]CC(N)═O) C(N[C@@H](Cc3cnccc3)C(N(C)CC(N)═ O)═O)═O)═O)═O)═O)═O)N3)cc2)═ O)═O)NC(CCCCC[N+](C)(C)C)═O) C3═O)═O)═O)═O)NC1═O)O |
| | MeCO-Pen(3)-E(2)-T-7MeW-hK(Me)3-Pen(3)- | |
| | AEF(NMe)(2)-2Nal-THP-E-N-3Pya-Sar- | |
| | CON(Me)2 | |
| 236 | 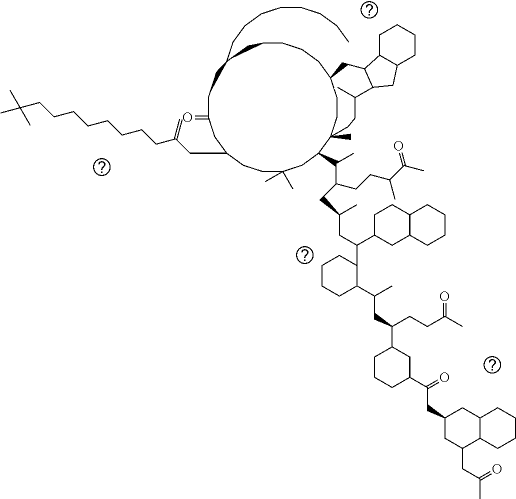 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCCNC(CCC[C@@H] (C(N[C@@H](CC(N)═O)C(N[C@@H] (Cc2cnccc2)C(N(C)CC(N)═O)═O)═O)═ O)NC(C2(CCOCC2)NC([C@H](Cc2cc3 ccccc3cc2)NC([C@H](Cc(cc2)ccc2OCC N)N2)═O)═O)═O)═O)NC(CCCCC[N+] (C)(C)C)═O)═O)C2═O)═O)═O)═O) NC1═O)O |
| | cPEG3aCO-Pen(3)-E(2)-T-7MeW-K(NMeAc)- | |
| | Pen(3)-AEF(NMe)(2)-2Nal-THP-E-N-3Pya-Sar- | |
| | CON(Me)2 | |
| 237 | 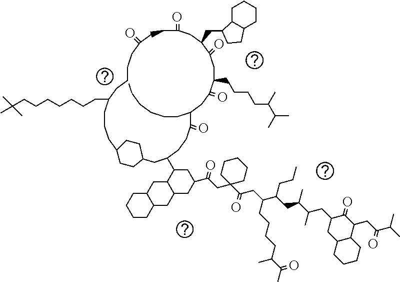 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCCCNC(C)═O)C(N[C@@H](CC (N)═O)C(N[C@@H](Cc3cnccc3)C(N(C)C C(N)═O)═O)═O)═O)═O)═O)═O)N3) cc2)═O)═O)NC(CCOCCOCC[N+](C) (C)C)═O)C3═O)═O)═O)═O)NC1═O)O |
| | cPEG3aCO-Pen(3)-E(2)-T-7MeW-K(NMeAc)- | |
| | Pen(3)-AEF(NMe)(2)-2Nal-THP-K(NMeAc)-N- | |
| | 3Pya-Sar-CON(Me)2 | |
| 238 | 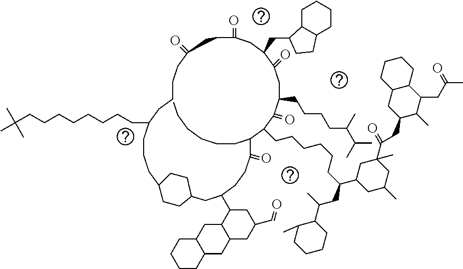 | C[C@H]([C@@H](C(N[C@@H](Cc1c [nH]c2c1cccc2)C(N[C@@H](CCCCNC (C)═O)C(N[C@@H](C(C)(C)SSC(C)(C) [C@@H](C(N[C@H]1CCC(NCCOc2cc c(C[C@@H](C(N[C@@H](Cc3cc4cccc c4cc3)C(NC3(CCOCC3)C(N[C@@H] (CCCCNC(C)═O)C(N[C@@H](CO)C(N [C@@H](Cc3cnccc3)C(N(C)CC(N)═O)═ O)═O)═O)═O)═O)═O)N3)cc2)═O)═O) NC(C)═O)C3═O)═O)═O)═O)NC1═O)O |
| | cPEG3aCO-Pen(3)-E(2)-T-7MeW-K(NMeAc)- | |
| | Pen(3)-AEF(2)-2Nal-THP-K(Ac)-N-3Pya-Sar- | |
| | CONH2 | |
| 239 | 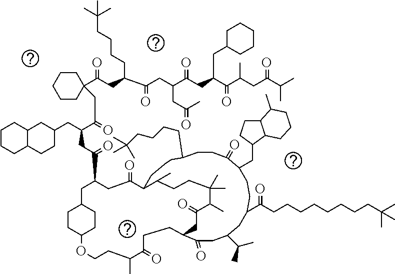 | C[C@H]([C@@H](C(N[C@@H](Cc1c [nH]c2c1cccc2C)C(N[C@@H](CCCCN C(C)═O)C(N[C@H](C(C)(C)SSC(C)(C) [C@@H](C(N[C@H]1CC(N)═O)═O)N C([C@H](CCC(NCCCC[C@H](C(N)═ O)NC([C@H](Cc2cnccc2)NC([C@H](C C(N)═O)NC([C@H](CCCCNC(C)═O)N C(C2(CCOCC2)NC([C@H](Cc2cc3cccc c3cc2)NC([C@H](Cc(cc2)ccc2OCCN)N 2)═O)═O)═O)═O)═O)═O)═O)NC(C)═ O)═O)C2═O)═O)═O)═O)NC1═O)O |
| | cPEG3aCO-Pen(3)-E(2)-T-7MeW-K(Me)3-Pen(3)- | |
| | AEF(NMe)(2)-2Nal-THP-K(Me)3-N-3Pya-Sar- | |
| | CON(Me)2 | |
| 240 | 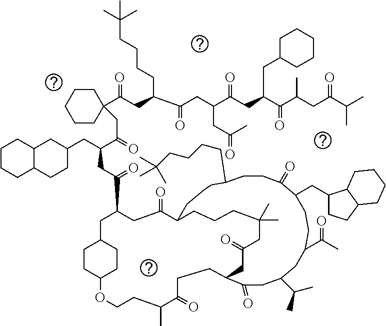 | C[C@H]([C@@H](C(N[C@@H](Cc1c [nH]c2c1cccc2C)C(N[C@@H](CCCCC[ N+](C)(C)C)C(N[C@@H](C(C)(C)SSC (C)(C)[C@@H](C(N[C@H]1CCC(N(C) CCOc2ccc(C[C@@H](C(N[C@@H](C c3cc4ccccc4cc3)C(NC3(CCOCC3)C(N[ C@@H](CCC(O)═O)C(N[C@@H](CC (N)═O)C(N[C@@H](Cc3cnccc3)C(N(C) CC(N(C)C)═O)═O)═O)═O)═O)═O)═O) N3)cc2)═O)═O)NC(C)═O)C3═O)═O)═ O)═O)NC1═O)O |
| | MeCO-Pen(3)-E(2)-T-7MeW-K(Me)3-Pen(3)- | |
| | AEF(NMe)(2)-2Nal-THP-K(Me)3-N-3Pya-Sar- | |
| | CON(Me)2 | |
| 241 | 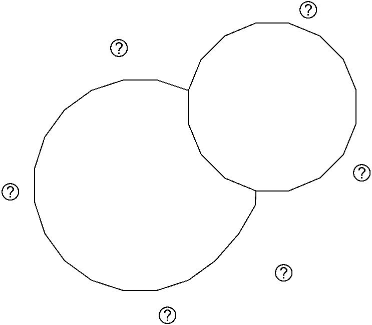 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN C)C(C)═O)C(N[C@@H](C(C)(C)SSC (C)(C)[C@@H](C(N[C@H]1CCC(N(C) CCOc2ccc(C[C@@H](C(N[C@@H](C c3cc4ccccc4cc3)C(NC3(CCOCC3)C(N[ C@@H](CCC(O)═O)C(N[C@@H](CC N)═O)C(N[C@@H](Cc3cnccc3)C(N(C) CC(N(C)C)═O)═O)═O)═O)═O)═O)═O) N3)cc2)═O)═O)NC(CCOCCOCC[N+] (C)(C)C)═O)C3═O)═O)═O)═O)NC1═ O)O |
| | cPEG3aCO-k(2)-Pen(3)-N-T-7MeW-K(Ac)-Pen(3)- | |
| | AEF-2Nal-THP-hE(2)-N-3Pya-Sar-CONH2 | |
| 242 | 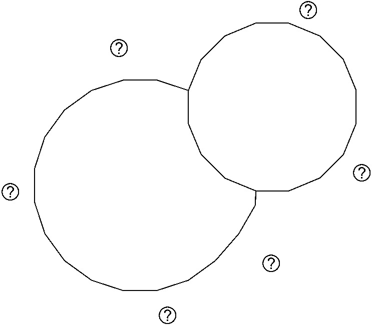 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CCC(N(C) CCOc2ccc(C[C@@H](C(N[C@@H](C c3cc4ccccc4cc3)C(NC3(CCOCC3)C(N [C@@H](CCCCN(C)C(C)═O)C(N[C@ @H](CC(N)═O)C(N[C@@H](Cc3cnccc 3)C(N(C)CC(N(C)C)═O)═O)═O)═O)═ O)═O)═O)N3)cc2)═O)═O)NC(CCOCCO CC[N+](C)(C)C)═O)C3═O)═O)═O)═O) NC1═O)O |
| | cPEG3aCO-DLys(2)-Pen(3)-N-T-7MeW- | |
| | K(NMeAc)-Pen(3)-AEF-2Nal-THP-hE(2)-N-3Pya- | |
| | Sar-CONH2 | |
| 243 | 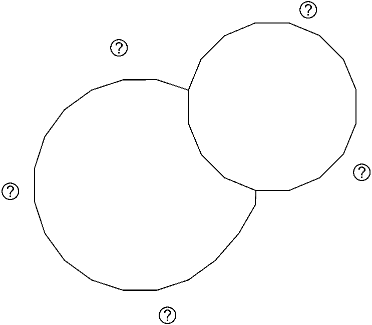 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CCC(NCC Oc2ccc(C[C@@H](C(N[C@@H](Cc3c c4ccccc4cc3)C(NC3(CCOCC3)C(N[C@ @H](CCCCNC(C)═O)C(N[C@@H](C C(N)═O)C(N[C@@H](Cc3cnccc3)C(N (C)CC(N)═O)═O)═O)═O)═O)═O)═O) N3)cc2)═O)═O)NC(CCOCCOCC[N+] (C)(C)C)═O)C3═O)═O)═O)═O)NC1═ O)O |
| | PEG2(2)-Pen(3)-N-T-7MeW-K(NMeAc)-Pen(3)- | |
| | AEF-2Nal-THP-hE(2)-N-3Pya-Sar-CONH2 | |
| 244 | 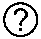 | C[C@H]([C@@H](C(N[C@@H](Cc1c [nH]c2c1cccc2C)C(N[C@@H](CCCC[N+] (C)(C)C)C(N[C@@H](C(C)(C)SSC(C) (C)[C@@H](C(N[C@H]1CCC(N(C) CCOc2ccc(C[C@@H](C(N[C@@H](C c3cc4ccccc4cc3)C(NC3(CCOCC3)C(N [C@@H](CCCC[N+](C)(C)C)C(N[C@ @H](CC(N)═O)C(N[C@@H](Cc3cnccc 3)C(N(C)CC(N(C)C)═O)═O)═O)═O)═ O)═O)═O)N3)cc2)═O)═O)NC(CCOCCO CC[N+](C)(C)C)═O)C3═O)═O)═O)═O) NC1═O)O |
| | MeCO-Pen(3)-E(2)-T-7MeW-K(mPEG12)-Pen(3)- | |
| | AEF(2)-2Nal-THP-K(Ac)-L-3Pya-Sar-CONH2 | |
| 245 | 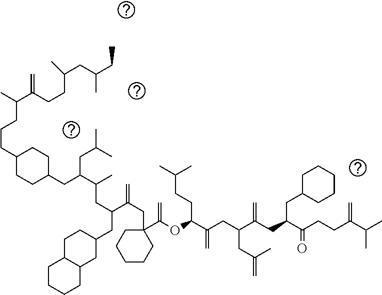 | C[C@H]([C@@H](C(N[C@@H](Cc1c [nH]c2c1cccc2C)C(N[C@@H](CCCC[N+] (C)(C)C)C(N[C@@H](C(C)(C)SSC(C) (C)[C@@H](C(N[C@H]1CCC(N(C) CCOc2ccc(C[C@@H](C(N[C@@H](C c3cc4ccccc4cc3)C(NC3(CCOCC3)C(N [C@@H](CCCC[N+](C)(C)C)C(N[C@ @H](CC(N)═O)C(N[C@@H](Cc3cnccc 3)C(N(C)CC(N(C)C)═O)═O)═O)═O)═ O)═O)N3)cc2)═O)═O)NC(C)═O)C3═ O)═O)═O)═O)NC1═O)O |
| | MeCO-k(Me)3-Pen(3)-E(2)-T-7MeW-K(Ac)- | |
| | Pen(3)-AEF(NMe)(2)-2Nal-THP-E-N-3Pya-Sar- | |
| | CON(Me)2 | |
| 246 | 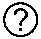 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC([C@@H](CCCCNC(CCC[C@@H] (C(N[C@@H](CC(N)═O)C(N[C@@H] (Cc2cnccc2)C(N(C)CC(N)═O)═O)═O)═ O)NC(C2(CCOCC2)NC([C@H](Cc2cc3 ccccc3cc2)NC([C@H](Cc(cc2)ccc2OCC N)N2)═O)═O)═O)═O)NC(CCOCCOCC [N+](C)(C)C)═O)═O)C2═O)═O)═O)═O) NC1═O)O |
| | 7Ahp(2)-Pen(3)-K(PEG2PEG2gEC18OH)-T- | |
| | 7MeW-K(Ac)-Pen(3)-AEF-2Nal-THP-E(2)-N- | |
| | 3Pya-Sar-CONH2 | |
| 247 | 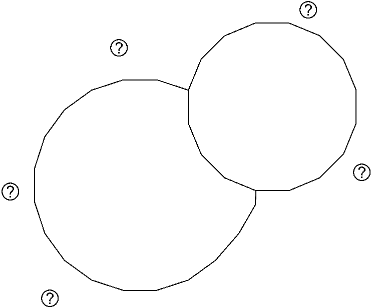 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CC(N)═ O)═O)NC([C@@H](CCCCNC(CCC[C@ @H](C(N([C@@H](CC(N)═O)C(N[C@ @H](Cc2cnccc2)C(N(C)CC(N)═O)═ O)═O)═O)NC(C2(CCOCC2)NC([C@H] (Cc2cc3ccccc3cc2)NC([C@H](Cc(cc2)ccc 2OCCN)N2)═O)═O)═O)═O)NC(CCOC COCC[N+](C)(C)C)═O)═O)C2═O)═O)═ O)═O)NC1═O)O |
| | AEEP(2)-Pen(3)-N-T-7MeW-K(Ac)-Pen(3)-AEF- | |
| | 2Nal-THP-E(2)-N-3Pya-Sar-CONH2 | |
| 248 | 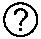 | C[C@H]([C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CC(N)═ O)═O)NC(COCCOCCNC(CCC[C@@H] (C(N[C@@H](CC(N)═O)C(N[C@@H] (Cc2cnccc2)C(N(C)CC(N)═O)═O)═O)═ O)NC(C2(CCOCC2)NC([C@H](Cc2cc3 ccccc3cc2)NC([C@H](Cc(cc2)ccc2OCC N)N2)═O)═O)═O)═O)═O)C2═O)═O)═ O)═O)NC1═O)O |
| | GABA(2)-k(PEG2PEG2gEC18OH)-Pen(3)-N-T- | |
| | 7MeW-K(Ac)-Pen(3)-AEF-2Nal-THP-E(2)-N- | |
| | 3Pya-Sar-CONH2 | |
| 249 | 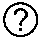 | CC(C)C[C@@H](C(N[C@@H](Cc1cnc cc1)C(N(C)CC(N)═O)═O)═O)NC([C@ H](CCCCNC(C)═O)NC(C1(CCOCC1) NC([C@H](Cc1cc2ccccc2cc1)NC([C@ H](Cc(cc1)ccc1OCCNC(CC[C@@H](C (N[C@@H]([C@@H](C)O)C(N[C@@ H](Cc1c[nH]c2c1cccc2C)C(N[C@@H]( CCCCNC(CCOCCOCCOCCOCCOCC OCCOCCOCCOCCOCCOCCOC)═O)C (N[C@H]1C(C)(C)SSC(C)(C)[C@@H] 2NC(C)═O)═O)═O)═O)═O)NC2═O)═O) NC1═O)═O)═O)═O)═O |
| | 5Ava(2)-Abu(1)-N-T-W-K(PEG2PEG2gEC18OH)- | |
| | C(1)-AEF-2Nal-THP-E(2)-N-3Pya-Sar-CONH2 | |
| 250 | 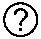 | C[C@H]([C@@H](C(N[C@@H](Cc1c nH]c2c1cccc2C)C(N[C@@H](CCCCN (C)C(C)═O)C(N[C@@H](C(C)(C)SSC (C)(C)[C@@H](C(N[C@H]1CCC(N(C) CCOc2ccc(C[C@@H](C(N[C@@H](C c3cc4ccccc4cc3)C(NC3(CCOCC3)C(N [C@@H](CCC(O)═O)C(N[C@@H](CC (N)═O)C(N[C@@H](Cc3cnccc3)C(N(C) CC(N(C)C)═O)═O)═O)═O)═O)═O)═O) N3)cc2)═O)═O)NC([C@@H](CCCC[N+] (C)(C)C)NC(C)═O)═O)C3═O)═O)═ O)═O)NC1═O)O |
| | 5Ava(2)-Abu(1)-N-T-W-Q-C(1)-AEF-2Nal-THP- | |
| | E(2)-N-3Pya-Sar-K(PEG2PEG2gEC18OH)- | |
| | CONH2 | |
| 251 | 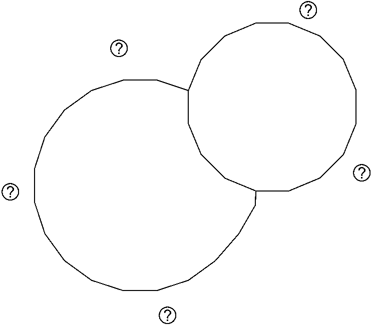 [SMILES: C1CCCCCCCCC2CCCCCCCCCCCC(CCCCCCC1)CCC2] | C[C@H]([C@@H](C(N[C@@H](Cc1c [nHc2c1cccc2C)C(N[C@@H](CCCCN C(C)═O)C(N[C@@H](C(C)(C)SSC(C) (C)[C@@H](C(N[C@H]1CC(N)═O)═O) NC(C)═O)C(N[C@@H](Cc(cc2)ccc2O CCN)C(N[C@@H](Cc(ccc2n3)cc2ccc3 OC)C(NC2(CCOCC2)C(N[C@@H](CC C(O)═O)C(N[C@@H](CC(N)═O)C(N [C@@H](Cc2cnccc2)C(N(C)CC(N)═O)═ O)═O)═O)═O)═O)═O)═O)═O)═O)═O) NC1═O)O |
| | 5Ava(2)-Abu(1)-N-T-W-Q-C(1)-AEF-2Nal-THP- | |
| | E(2)-N-H-Sar-CONH2 | |
| 252 | 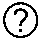 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC(CCOCCOCCNC(CC[C@@H](C(N [C@@H](CC(N)═O)C(N[C@@H](Cc2c nccc2)C(N(C)CC(N)═O)═O)═O)═O)NC (C2(CCOCC2)NC([C@H](Cc2cc3ccccc 3cc2)NC([C@H](Cc(cc2)ccc2OCCN)N 2)═O)═O)═O)═O)═O)C2═O)═O)═O)═ O)NC1═O)O |
| | 6Ahx(2)-Abu(1)-N-T-W-Q-C(1)-AEF-2Nal-THP- | |
| | E(2)-N-3Pya-NMeK(PEG2PEG2gEC18OH)- | |
| | CONH2 | |
| 253 | 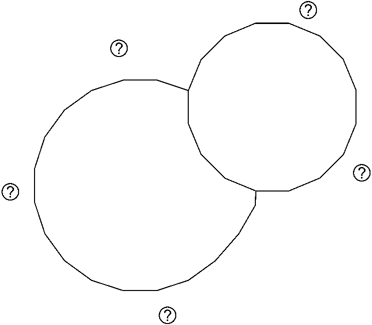 [SMILES: C1CCCCCCCCC2CCCCCCCCCCCC(CCCCCCC1)CCC2] | C[C@H]([C@@H](C(N[C@@H](Cc1c [nH]c2c1cccc2)C(N[C@@H](CCCCNC( (COCCOCCNC(COCCOCCNC(CC[C@ @H](C(O)═O)NC(CCCCCCCCCCCCC CCCC(O)═O)═O)═O)═O)═O)C(N[C@ @H](CSCC[C@@H](C(N[C@H]1CC (N)═O)═O)NC(CCCCNC(CC[C@@H] (C(N[C@@H](CC(N)═O)C(N[C@@H] (Cc2cnccc2)C(N(C)CC(N)═O)═O)═O)═ O)NC(C2(CCOCC2)NC([C@H](Cc2cc3 ccccc3cc2)NC([C@H](Cc(cc2)ccc2OCC N)N2)═O)═O)═O)═O)═O)C2═O)═O)═ O)═O)NC1═O)O |
| | 5Ava(2)-Abu(1)-N-T-W-Q-C(1)-AEF-2Nal-THP- | |
| | E(2)-N-3MeH-Sar-CONH2 | |
| 254 | 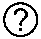 | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCNC(CC [C@@H](C(N[(C@@H](CC(N)═O)C(N [C@@H](Cc2cnccc2)C(N(C)CC(N[C@ @H](CCCCNC(COCCOCCNC(COCC OCCNC(CC[C@@H](C(O)═O)NC(CC CCCCCCCCCCCCCCC(O)═O)═O)═O)═ O)═O)C(N)═O)═O)═O)═O)═O)NC(C2 (CCOCC2)NC([C@H](Cc2cc3ccccc3cc 2)NC([C@H](Cc(cc2)ccc2OCCN)N2)═ O)═O)═O)═O)═O)C2═O)═O)═O)═O)N C1═O)O |
| | AEEP(2)-Pen(3)-N-T-7MeW-K(Ac)-Pen(3)-AEF- | |
| | 2Nal-THP-E(2)-N-3Pya-Sar- | |
| | K(PEG2PEG2gEC18OH)-CONH2 | |
| 255 | 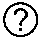 | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCNC(CC [C@@H](C(N[C@@H](CC(N)═O)C(N [C@@H](Cc2cnc[nH]2)C(N(C)CC(N)═ O)═O)═O)═O)NC(C2(CCOCC2)NC([C @H](Cc2cc3ccccc3cc2)NC([C@H](Cc (cc2)ccc2OCCN)N2)═O)═O)═O)═O)═O) C2═O)═O)═O)═O)NC1═O)O |
| | 6Ahx(2)-Abu(1)-K(PEG2PEG2gEC18OH)-T-W-Q- | |
| | C(1)-AEF-2Nal-THP-E(2)-N-3Pya-Sar-CONH2 | |
| 256 | 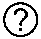 | C[C@H]([C@@H](C(N[C@@H](Cc1c [nH]c2c1cccc2)C(N[C@@H](CCC(N)═ O)C(N[C@@H](CSCC[C@@H](C(N[C @H]1CC(N)═O)═O)NC(CCCCNC(CC[ [C@@H](C(N[C@@H](CC(N)═O)C(N[ [C@@H](Cc2cncn2C)C(N(C)CC(N)═ O)═O)═O)═O)NC(C2(CCOCC2)NC([C@ H](Cc2cc3ccccc3cc2)NC([C@H] (Cc(cc 2)ccc2OCCN)N2)═O)═O)═O)═O)═O)C 2═O)═O)═O)═O)NC1═O)O |
| | HOC18gEPEG2PEG2CO-r-Pen(3)-E(2)-T-7MeW- | |
| | K(Ac)-Pen(3)-AEF(2)-2Nal-THP-E-N-3Pya-Sar- | |
| | CONH2 | |
| 257 | 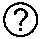 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC(CCOCCOCCNC(CC[C@@H](C(N [C@@H](CC(N)═O)C(N[C@@H](Cc2c nccc2)C(N(C)CC(N[C@@H](CCCCNC (COCCOCCNC(COCCOCCNC(CC[C @@H](C(O)═O)NC(CCCCCCCCCCC CCCCCC(O)═O)═O)═O)═O)═O)C(N)═ O)═O)═O)═O)═O)NC(C2(CCOCC2)NC ([C@H](Cc2cc3ccccc3cc2)NC([C@H] (Cc(cc2)ccc2OCCN)N2)═O)═O)═O)═ O)═O)C2═O)═O)═O)═O)NC1═O)O |
| | 5Ava(2)-Abu(1)-K(PEG2PEG2gEC18OH)-T-W-Q- | |
| | C(1)-AEF-2Nal-THP-E(2)-N-3Pya-Sar-CONH2 | |
| 258 | 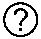 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCC(O)═O)C(N[C@@H](CC(N)═O) C(N[C@@H](Cc3cnccc3)C(N(C)CC (N)═O)═O)═O)═O)═O)═O)═O)N3) cc2)═O)═O)NC([C@@H](CCCNC (N)═N)NC(COCCOCCNC(COCCOCC NC(CC[C@@H](C(O)═O)NC(CCCCC CCCCCCCCCO)═O)═O)NC1═O)O |
| | MeCO-k(PEG2PEG2gEC18OH)-Pen(3)-E(2)-T- | |
| | 7MeW-K(Ac)-Pen(3)-AEF(2)-2Nal-THP-E-N- | |
| | 3Pya-Sar-CONH2 | |
| 259 | 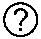 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCC(O)═O)C(NIC@@H](CC(N)═O) C(N[C@@H](Cc3cnccc3)C(N(C)CC(N)═ O)═O)═O)═O)═O)═O)═O)N3)cc2)═O)═ O)NC([C@@H](CCCCNC(COCCOC CNC(COCCOCCNC(CC[C@@H](C(O)═ O)NC(CCCCCCCCCCCCCCCCC(O)═ O)═O)═O)═O)═O)NC(C)═O)═O)C3═ O)═O)═O)═O)NC1═O)O |
| | HOC16gEPEG2PEG2orn(2)-r-Pen(3)-N-T-7MeW- | |
| | K(Ac)-Pen(3)-AEF-2Nal-THP-E(2)-N-3Pya-Sar- | |
| | CONH2 | |
| 260 | 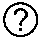 | C[C@H]([C@@H](C(N[C@@H](Cc1c [nH]c2c1cccc2C)C(N[C@@H](CCCCN C(C)═O)C(N[C@@H](C(C)(C)SSC(C)( C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCC(O)═O)C(N[C@@H](CC(N)═O) C(N[C@@H](Cc3cnccc3)C(N(C)CC(N [C@@H](CCCCNC(COCCOCCNC(CO CCOCCNC(CC[C@@H](C(O)═O)NC (CCCCCCCCCCCCCCCCC(O)═O)═O)═ O)═O)═O)C(N)═O)═O)═O)═O)═O)═ O)═O)═O)N3)cc2)═O)═O)NC([C@@H] (CCCNC(N)═N)NC(C)═O)═O)C3═O)═ O)═O)═O)NC1═O)O |
| | MeCO-r-Pen(3)-E(2)-T-7MeW-K(Ac)-Pen(3)- | |
| | AEF(2)-2Nal-THP-E-N-3Pya-Sar- | |
| | K(PEG2PEG2gEC18OH)-CONH2 | |
| 261 | 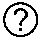 | C[C@H]([C@@H](C(N[C@@H](Cc1c [nH]c2c1cccc2C)C(N[C@@H](CCCCN C(COCCOCCNC(COCCOCCNC(CC[C @@H](C(O)═O)NC(CCCCCCCCCCC CCCCCC(O)═O)═O)═O)═O)═O)C(N[C @@H(C(C)(C)SSC(C)(C)[C@@H](C (N[C@H]1CC(N)═O)═O)NC(CCOCCO CCNC(CC[C@@H](C(N[C@@H](CC (N)═O)C(N[C@@H](Cc2cnccc2)C(N(C) CC(N)═O)═O)═O)═O)NC(C2(CCOCC2) NC([C@H](Cc2cc3ccccc3cc2)NC([C@ H](Cc(cc2)ccc2OCCN)N2)═O)═O)═O)═ O)═O)C2═O)═O)═O)═O)NC1═O)O |
| | AEEP(2)-Pen(3)-N-T-7MeW- | |
| | K(PEG2PEG2gEC18OH)-Pen(3)-AEF-2Nal-THP- | |
| | E(2)-N-3Pya-Sar-CONH2 | |
| 262 | 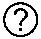 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCCCNC(CO CCOCCNC(COCCOCCNC(CC[C@@H] (C(O)═O)NC(CCCCCCCCCCCCCCC CC(O)═O)═O)═O)═O)═O)═O)NC(CCO CCOCCNC(CC[C@@H](C(N[C@@H] (CC(N)═O)C(N[C@@H](Cc2cnccc2)C (N(C)CC(N)═O)═O)═O)═O)NC(C2(CCO CC2)NC([C@H](Cc2cc3ccccc3cc2)NC ([C@H](Cc(cc2)ccc2OCCN)N2)═O)═ O)═O)═O)═O)C2═O)═O)═O)═O)NC1═ O)O |
| | AEEP(2)-Pen(3)-K(PEG2PEG2gEC18OH)-T- | |
| | 7MeW-K(Ac)-Pen(3)-AEF-2Nal-THP-E(2)-N- | |
| | 3Pya-Sar-CONH2 | |
| 263 | 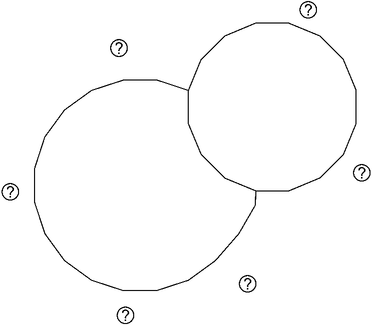 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CC(N(C) C)═O)═O)NC([C@@H](CCCC(N(C)CC CC[C@@H](C(N[C@@H](CC(N)═O)C (N[C@@H](Cc2cnccc2)C(N(C)CC(N(C) C)═O)═O)═O)═O)NC(C2(CCOCC2)NC ([C@H](Cc2cc3ccccc3cc2)NC([C@H] (Cc(cc2)ccc2OCCCCC[N+](C)(C)C) N2)═O)═O)═O)═O)NC(CCOCCOCC [N+](C)(C)C)═O)═O)C2═O)═O)═O)═ O)NC1═O)O |
| | cPEG3aCO-dhE(2)-Pen(3)-N(N(Me)2)-T-7MeW- | |
| | K(NMeAc)-Pen(3)-TMAPF-2Nal-THP-KNMe(2)- | |
| | N-3Pya-Sar-CON(Me)2 | |
| 264 | 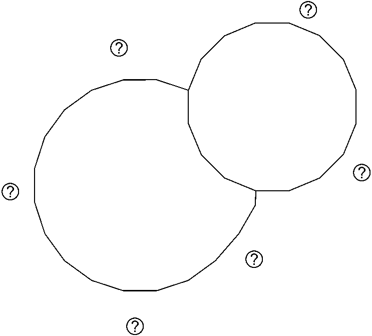 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN (C)C(C)═O)C(N[C@@H](C(C)(C)SSC (C)(C)[C@@H](C(N[C@H]1CC(N(C) C)═O)═O)NC([C@@H](CCCCNC(CCC [C@@H](C(N[C@@H](CC(N)═O)C(N [C@@H](Cc2cnccc2)C(N(C)CC(N(C) C)═O)═O)═O)═O)NC(C2(CCOCC2)NC ([C@H](Cc2cc3ccccc3cc2)NC([C@H](C c(cc2)ccc2OCCCCC[N+](C)(C)C)N2)═ O)═O)═O)═O)NC(CCOCCOCC[N+](C) (C)C)═O)═O)C2═O)═O)═O)═O)NC1═ O)O |
| | cPEG3aCO-k(2)-Pen(3)-N(N(Me)2)-T-7MeW- | |
| | K(NMeAc)-Pen(3)-TMAPF-2Nal-THP-hE(2)-N- | |
| | 3Pya-Sar-CON(Me)2 | |
| 265 | 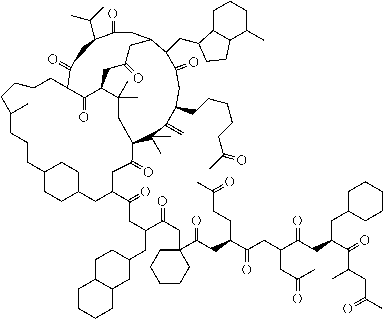 [SMILES: C=C1[C@H](CCCCCC(C)=O)CC(=O)C(CC2CCC3C(C)CCCC23)C2CC(=O)C[C@@H]3C(=O)C(CCCCC(C)CCCC4CCC(CC4)CC(C(=O)CC(CC4CCC5CCCCC5C4)C(=O)CC4(C(=O)C[C@@H](CCC(C)=O)C(=O)CC(CC(C)=O)C(=O)C[C@@H](CC5CCCCC5)C(=O)C(C)CC(C)=O)CCCCC4)CC(=O)[C@@H](CC3(C)C)C1(C)C)C(=O)C[C@@H](C(C)C)C(=O)C2] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCCCN(C)CC Oc2ccc(C[C@@H](C(N[C@@H](Cc3c c4ccccc4cc3)C(NC3(CCOCC3)C(N[C@ @H](CCC(O)═O)C(N[C@@H](CC (N)═O)C(N[C@@H](Cc3cnccc3)C(N(C)C C(N)═O)═O)═O)═O)═O)═O)═O)N3) cc2)═O)NC(C)═O)C3═O)═O)═O)═O) NC1═O)O |
| | MeCO-Pen(3)-K(NMe)(5)-T-7MeW-K(Ac)-Pen(3)- | |
| | AEF(5)-2Nal-THP-E-N-3Pya-Sar-CONH2 | |
| 266 | 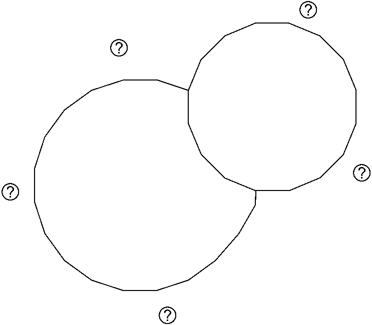 [SMILES: C1CCCCCCCCC2CCCCCCCCCCCC(CCCCCCC1)CCC2] | C[C@H]([C@@H](C(N[C@@H](Cc1c [nH]c2c1cccc2)C(N[C@@H](CCC(N)═ O)C(N[C@@H](C(C)(C)SSC(C)(C)[C @@H](C(N[C@H]1CCC(N)═O)═O)NC ([C@@H](CCCNC(N)═N)NC(CCCNC (CC[C@@H](C(N[C@@H](CC(N)═O)C (NC2(CCOCC2)C(N)═O)═O)═O)NC(C 2(CCOCC2)NC([C@H](Cc2cc3ccccc3c c2)NC([C@H](Cc(cc2)ccc2OCCN)N2)═ O)═O)═O)═O)═O)═O)C2═O)═O)═O)═ O)NC1═O)O |
| | GABA(2)-r-Pen(3)-Q-T-W-Q-Pen(3)-AEF-2Nal- | |
| | THP-E(2)-N-THP-CONH2 | |
| 267 | 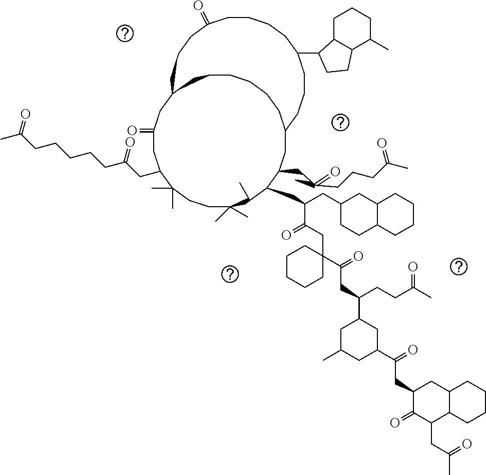 | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1ccc(NCCOc2 ccc(C[C@@H](C(N[C@@H](Cc3cc4cc ccc4cc3)C(NC3(CCOCC3)C(N[C@@H] (CCC(O)═O)C(N[C@@H](CC(N)═O) C(N[C@@H](Cc3cnccc3)C(N(C)CC (N)═O)═O)═O)═O)═O)═O)═O)N3) cc2)═O)═O)NC([C@@H](CCCNC(N)═ N)NC(C)═O)═O)C3═O)═O)═O)═O) NC1═O)O |
| | MeCO-r-Pen(3)-E(2)-T-7MeW-K(Ac)-Pen(3)- | |
| | AEF(2)-2Nal-THP-E-N-3Pya-Sar-CONH2 | |
| 268 | 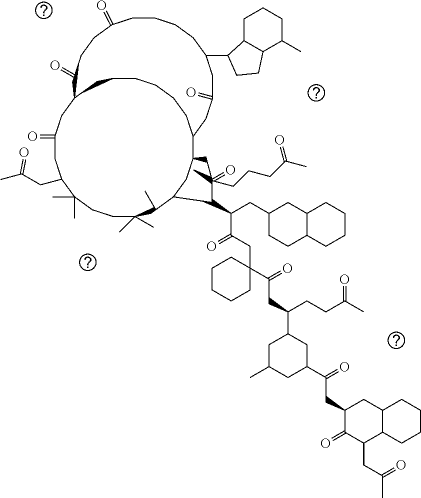 | C[C@H][C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CCC(NCCOc2 ccc(C[C@@H](C(N[C@@H](Cc3cc4cc ccc4cc3)C(NC3(CCOCC3)C(N[C@@H] (CCC(O)═O)C(N[C@@H](CC(N)═O) C(N[C@@H](Cc3cnccc3)C(N(C)CC (N)═O)═O)═O)═O)═O)═O)═O)N3) cc2)═O)═O)NC(C)═O)C3═O)═O)═ O)═O)NC1═O)O |
| | MeCO-Pen(3)-E(2)-T-7MeW-K(Ac)-Pen(3)- | |
| | AEF(2)-2Nal-THP-E-N-3Pya-Sar-CONH2 | |
| 269 | 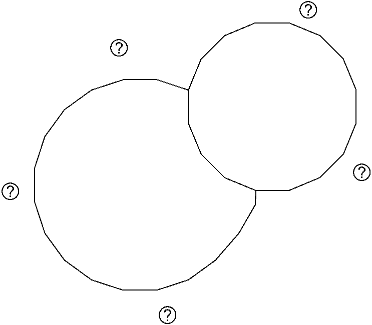 [SMILES: C1CCCCCCCCC2CCCCCCCCCCCC(CCCCCCC1)CCC2] | C[C@H][C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC([C@@H](CC NC(CC[C@@H](C(N[C@@H](CC(N)═ O)C(NCCC(N)═O)═O)═O)NC(C2(CCO CC2)NC([C@H](Cc2cc3ccccc3cc2)NC ([C@H](Cc(cc2)ccc2OCCN)N2)═O)═ O)═O)═O)NC(C)═O)═O)C2═O)═O)═ O)═O)NC1═O)O |
| | MeCO-dab(2)-Abu(1)-Q-T-W-Q-C(1)-AEF-2Nal- | |
| | THP-E(2)-N-bAla-CONH2 | |
| 270 | 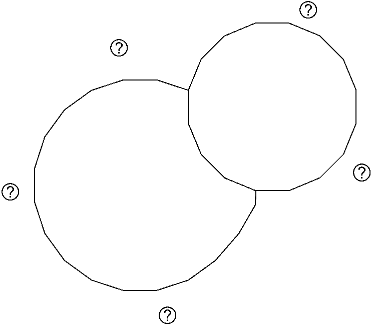 [SMILES: C1CCCCCCCCC2CCCCCCCCCCCC(CCCCCCC1)CCC2] | C[C@H][C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC([C@H](CCC CCNC(CC[C@@H](C(N[C@@H](CC( N)═O)C(N[C@@H](Cc2cnccc2)C(N(C) CC(N)═O)═O)═O)═O)NC(C2(CCOCC2) NC([C@H](Cc2cc3ccccc3cc2)NC([C@ H](Cc(cc2)ccc2OCCN)N2)═O)═O)═O)═ O)NC(C)═O)═O)C2═O)═O)═O)═O) NC1═O)O |
| | MeCO-hlys(2)-Abu(1)-Q-T-W-Q-C(1)-AEF-2Nal- | |
| | THP-E(2)-N-3Pya-Sar-CONH2 | |
| 271 | 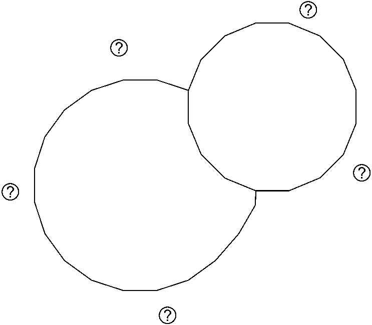 [SMILES: C1CCCCCCCCC2CCCCCCCCCCCC(CCCCCCC1)CCC2] | C[C@H]([C@@H](C(N[C@@H](Cc1c [nH]c2c1cccc2)C(N[C@@H](CCC(N)═ O)C(N[C@@H](CSCC[C@@H](C(N[C @H]1CCC(N)═O)═O)NC([C@@H](CC NC(CC[C@@H](C(N[C@@H](CC(N)═ O)C(N[C@@H](Cc2cnccc2)C(N(C)CC (N)═O)═O)═O)═O)NC(C2(CCOCC2)NC ([C@H](Cc2cc3ccccc3cc2)NC([C@H]( Cc(cc2)ccc2OCCN)N2)═O)═O)═O)═O) NC(C)═O)═O)C2═O)═O)═O)═O)NC1═ O)O |
| | MeCO-dab(2)-Abu(1)-Q-T-W-Q-C(1)-AEF-2Nal- | |
| | THP-E(2)-N-3Pya-Sar-CONH2 | |
| 272 | 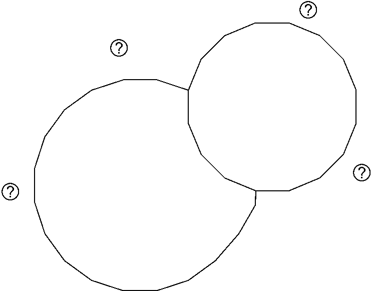 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC(CCOCCOCCNCC[C@@H](C(N[C @@H](CC(N)═O)C(N[C@@H](Cc2cnc cc2)C(N(C)CC(N)═O)═O)═O)═O)NC(C 2(CCOCC2)NC([C@H](Cc2cc3ccccc3c c2)NC([C@H](Cc(cc2)ccc2OCCN)N2)═ O)═O)═O)═O)C2═O)═O)═O)═O)NC1═ O)O |
| | AEEP(5)-Pen(3)-N-T-7MeW-K(Ac)-Pen(3)-AEF- | |
| | 2Nal-THP-hSer(5)-N-3Pya-Sar-CONH2 | |
| 273 | 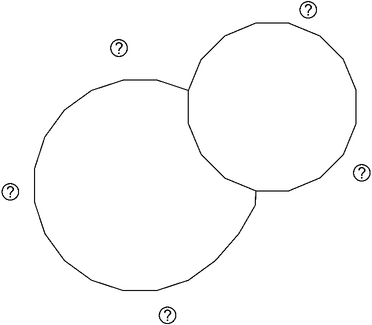 [SMILES: C1CCCCCCCCC2CCCCCCCCCCCC(CCCCCCC1)CCC2] | C[C@H]([C@@H](C(N[C@@H](Cc1c [nH]c2c1cccc2C)C(N[C@@H](CCCCN C(C)═O)C(N[C@@H](C(C)(C)SSC(C) (C)[C@@H](C(N[C@H]1CC(N)═O)═O) NC(CCOCCOCCN(CC[C@@H](C(N[C @@H](CC(N)═O)C(N[C@@H](Cc2cnc cc2)C(N(C)CC(N)═O)═O)═O)═O)NC(C 2(CCOCC2)NC([C@H](Cc2cc3ccccc3c c2)NC([C@H](Cc(cc2)ccc2OCCN)N2)═ O)═O)═O)S(c(cccc3)c3[N+]([O−])═ O)(═O)═O)═O)C2═O)═O)═O)═O)NC 1═O)O |
| | AEEP(Ns)(5)-Pen(3)-N-T-7MeW-K(Ac)-Pen(3)- | |
| | AEF-2Nal-THP-hSer(5)-N-3Pya-Sar-CONH2 | |

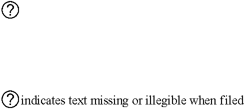

### Synthesis

The compounds described herein may be synthesized by many techniques that are known to those skilled in the art. In certain aspects, monomer subunits are synthesized and purified using the techniques described in the accompanying Examples. In some aspects, the present invention provides a method of producing a compound (or monomer subunit thereof) of the invention, comprising chemically synthesizing a peptide having an amino acid sequence described herein, including but not limited to any of the amino acid sequences set forth in the compounds of Formulas (I) to (XX), Table 1A, Table 1B, Table 1C, Table 1D, Table 1E, Table 1F, Table 1G and Table 1H herein. In some aspects, a portion of the peptide is recombinantly synthesized, instead of being chemically synthesized. In some aspects, methods of producing a compound further include cyclizing the compound precursor after the constituent subunits have been attached. In particular aspects, cyclization is accomplished via any of the various methods described herein.

The present invention further describes synthesis of compounds described herein, such as the compounds of Formulas (I) to (XX) and the compounds of Table 1A, Table 1B, Table 1C, Table 1D, Table 1E, Table 1F, Table 1G, and Table 1H. In some aspects, one or more of the amino acid residues or amino acid monomers are lipidated and then covalently attached to one another to form a compound of the invention. In some aspects, one or more of the amino acid residues or amino acid monomers are covalently attached to one another and lipidated at an intermediate oligomer stage before attaching additional amino acids and cyclization to form a compound of the invention. In some aspects, a cyclic peptide is synthesized and then lipidated to form a compound of the invention. Illustrative synthetic methods are described in the Examples.

The present invention further describes synthesis of compounds described herein, such as the compounds of Formulas (I) to (XX) and the compounds of Table 1A, Table 1B, Table 1C, Table 1D, Table 1E, Table 1F, Table 1G, and Table 1H. Illustrative synthetic methods are described in the Examples.

### IV. Pharmaceutical Compositions

The present invention relates to pharmaceutical composition which comprises an IL-23R inhibitor of the present invention.

The present invention includes pharmaceutical compositions comprising one or more inhibitors of the present invention and a pharmaceutically acceptable carrier, diluent or excipient.

The pharmaceutically acceptable carrier, diluent or excipient may be a solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The pharmaceutical compositions may be administered orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered orally. A pharmaceutical composition may be formulated for and administered parenterally.

In a particular aspects, an IL-23R inhibitor of the present invention, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

The IL-23R inhibitors of the present invention may be prepared and/or formulated as pharmaceutically acceptable salts or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

The present invention relates to pharmaceutical compositions comprising an IL-23R inhibitor of the present invention or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), can generally be prepared by conventional techniques known to those skilled in the art or by processes analogous to those described in the Examples as set out below using an appropriate isotopically-labeled reagent in place of the non-labeled reagent previously employed.

In some aspects, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, β-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminum monostearate and gelatin.

Injectable depot forms include those made by forming microencapsulated matrices of the peptide inhibitor in one or more biodegradable polymers such as polylactide-polyglycolide, poly(orthoesters), poly(anhydrides), and (poly)glycols, such as PEG. Depending upon the ratio of peptide to polymer and the nature of the particular polymer employed, the rate of release of the peptide inhibitor can be controlled. Depot injectable formulations are also prepared by entrapping the peptide inhibitor in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and non-aqueous formulations and can be prepared as a dry powder which may be pressurized or non-pressurized. In non-pressurized powder compositions, the active ingredient may be finely divided form may be used in admixture with a larger-sized pharmaceutically acceptable inert carrier comprising particles having a size, for example, of up to 100 micrometers in diameter. Suitable inert carriers include sugars such as lactose.

Alternatively, a pharmaceutical composition of the present invention may be pressurized and contain a compressed gas, such as nitrogen or a liquefied gas propellant. The liquefied propellant medium and indeed the total composition may be such that the active ingredient does not dissolve therein to any substantial extent. The pressurized composition may also contain a surface active agent, such as a liquid or solid non-ionic surface active agent or may be a solid anionic surface active agent. It is preferred to use the solid anionic surface active agent in the form of a sodium salt.

A further form of topical administration is to the eye. A peptide inhibitor of the present invention may be delivered in a pharmaceutically acceptable ophthalmic vehicle, such that the peptide inhibitor is maintained in contact with the ocular surface for a sufficient time period to allow the peptide inhibitor to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the peptide inhibitors of the invention may be injected directly into the vitreous and aqueous humor.

Compositions for rectal or vaginal administration include suppositories which may be prepared by mixing the peptide inhibitors of this invention with suitable non-irritating excipients or carriers such as cocoa butter, polyethylene glycol or a suppository wax, which are solid at room temperature but liquid at body temperature and, therefore, melt in the rectum or vaginal cavity and release the active compound.

Peptide inhibitors of the present invention may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a peptide inhibitor of the present invention, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions suitable for parenteral administration in a method or use described herein may comprise sterile aqueous solutions and/or suspensions of the IL-23R inhibitors made isotonic with the blood of the recipient, generally using sodium chloride, glycerin, glucose, mannitol, sorbitol, and the like.

The present invention provides a pharmaceutical composition for oral delivery. Compositions and peptide inhibitors of the present invention may be prepared for oral administration according to any of the methods, techniques, and/or delivery vehicles described herein. Further, one having skill in the art will appreciate that the peptide inhibitors of the instant invention may be modified or integrated into a system or delivery vehicle that is not disclosed herein, yet is well known in the art and compatible for use in oral delivery of peptides.

Formulations for oral administration may comprise adjuvants (e.g. resorcinols and/or nonionic surfactants such as polyoxyethylene oleyl ether and n-hexadecylpolyethylene ether) to artificially increase the permeability of the intestinal walls, and/or enzymatic inhibitors (e.g. pancreatic trypsin inhibitors, diisopropylfluorophosphate (DFF) or trasylol) to inhibit enzymatic degradation. In certain embodiments, the peptide inhibitor of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These formulations for oral administration can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

In particular aspects, oral dosage forms or unit doses compatible for use with the peptide inhibitors of the present invention may include a mixture of peptide inhibitor and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of peptide inhibitor, wherein the dosage form comprises at least one of a pill, a tablet, a capsule, a gel, a paste, a drink, a syrup, ointment, and suppository. In some instances, an oral dosage form is provided that is designed and configured to achieve delayed release of the peptide inhibitor in the subject's small intestine and/or colon.

Tablets may contain excipients, glidants, fillers, binders and the like. Aqueous compositions are prepared in sterile form, and when intended for delivery by other than oral administration generally will be isotonic. Compositions may optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the compositions ranges from, for example, about 3 to about 11. The pH of the compositions may, for example, range from about 5 to about 7 or from about 7 to about 10.

An oral pharmaceutical composition of the present invention may comprise an IL-23R inhibitor of the present invention may comprise an enteric coating that is designed to delay release of the IL-23R inhibitor in the small intestine. The present invention relates to a pharmaceutical composition that comprises an IL-23R inhibitor of the present invention and a protease inhibitor, such as aprotinin, in a delayed release pharmaceutical formulation. Pharmaceutical compositions (e.g., oral pharmaceutical compositions) may comprise an enteric coat that is soluble in gastric juice at a pH of about 5.0 or higher. Such enteric coatings may comprise a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

An oral pharmaceutical composition comprising an IL-23R inhibitor of the present invention that comprises an IL-23R inhibitor may comprise an enteric coating that is designed to protect and release the pharmaceutical composition in a controlled manner within the subject's lower gastrointestinal system, and to avoid systemic side effects. In addition to enteric coatings, the peptide inhibitors of the instant invention may be encapsulated, coated, engaged or otherwise associated within any compatible oral drug delivery system or component. For example, in some embodiments an IL-23R inhibitor of the present invention is provided in a lipid carrier system comprising at least one of polymeric hydrogels, nanoparticles, microspheres, micelles, and other lipid systems.

To overcome peptide degradation of an IL-23R inhibitor of the present invention in the small intestine, the pharmaceutical compositions may comprise a hydrogel polymer carrier system in which a peptide inhibitor of the present invention is contained, whereby the hydrogel polymer protects the IL-23R inhibitor from proteolysis in the small intestine and/or colon. The IL-23R inhibitor may further be formulated for compatible use with a carrier system that is designed to increase the dissolution kinetics and enhance intestinal absorption of the peptide. These methods include the use of liposomes, micelles and nanoparticles to increase GI tract permeation of peptides.

Various bioresponsive systems may also be combined with one or more an IL-23R inhibitors of the present invention to provide a pharmaceutical agent for oral delivery. For example, an IL-23R inhibitor of the present invention may be used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration.

In certain aspects, pharmaceutical composition and formulations may include an IL-23R inhibitor of the present invention and one or more absorption enhancers, enzyme inhibitors, or mucoso adhesive polymers. In an embodiment, the absorption enhancer may be an intestinal permeation enhancer.

IL-23R inhibitors of the present invention may be formulated in a formulation vehicle, such as, e.g., emulsions, liposomes, microsphere or nanoparticles.

The present invention provides for a method for treating a subject with an IL-23R inhibitor of the present invention having an increased half-life. In one aspect, the present invention provides a peptide inhibitor having a half-life of at least several hours to one day in vitro or in vivo (e.g., when administered to a human subject) sufficient for daily (q.d.) or twice daily (b.i.d.) dosing of a therapeutically effective amount. In certain embodiments, the IL-23R inhibitor has a half-life of three days or longer sufficient for weekly (q.w.) dosing of a therapeutically effective amount. In certain embodiments, the IL-23R inhibitor has a half-life of eight days or longer sufficient for bi-weekly (b.i.w.) or monthly dosing of a therapeutically effective amount. In certain embodiments, the IL-23R inhibitor is derivatized or modified such that is has a longer half-life as compared to the underivatized or unmodified peptide inhibitor. In certain embodiments, the IL-23R inhibitor contains one or more chemical modifications to increase serum half-life.

When used in at least one of the treatments or delivery systems described herein, a peptide inhibitor of the present invention may be employed in pure form or, where such forms exist, in pharmaceutically acceptable salt form.

The total daily usage of the IL-23R inhibitor and compositions of the present invention can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific peptide inhibitor employed; e) the duration of the treatment; f) drugs used in combination or coincidental with the specific peptide inhibitor employed, and like factors well known in the medical arts.

In particular embodiments, the total daily dose of a IL-23R inhibitor of the present invention to be administered to a human or other mammal host in single or divided doses may be in amounts, for example, from 0.0001 to 300 mg/kg body weight daily or 1 to 300 mg/kg body weight daily.

The compositions may conveniently be presented in unit dosage form and can be prepared by any of the methods well known in the art of pharmacy. Techniques and compositions generally are found in Remington's Pharmaceutical Sciences (Mack Publishing Co., Easton, PA). Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then, if necessary, shaping the product.

Compositions suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. The active ingredient may also be administered as a buccal or sublingual formulation. Buccal or sublingual formulations may comprise an active ingredient in a matrix that releases the active ingredient for transport across the buccal and/or sublingual membranes. The buccal or sublingual formulation may further include a rate controlling matrix that releases the active compounds at a predetermined rate for transport across the buccal and/or sublingual membranes. The buccal or sublingual formulation may further include one or more compounds selected from the group consisting of (i) taste masking agents, (ii) enhancers, (iii) complexing agents, and mixtures thereof; and (iv) other pharmaceutically acceptable carriers and/or excipients. The enhancer may be a permeation enhancer.

A tablet is made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets can optionally be coated or scored and optionally are formulated so as to provide slow or controlled release of the active ingredient therefrom.

### V. Non-Invasive Detection of Intestinal Inflammation

The IL-23R inhibitors of the present invention may be used for detection, assessment and diagnosis of intestinal inflammation by microPET imaging, wherein the peptide inhibitor is labeled with a chelating group or a detectable label, as part of a non-invasive diagnostic procedure. In certain embodiments, an IL-23R inhibitor of the present invention is conjugated with a bifunctional chelator. In certain embodiments, an IL-23R inhibitor of the present invention is radiolabeled. The labeled an IL-23R inhibitor is then administered to a subject orally or rectally. In certain embodiments, an IL-23R inhibitor is included in drinking water. Following uptake of the IL-23R inhibitor, microPET imaging may be used to visualize inflammation throughout the subject's bowels and digestive track.

### VI. Methods of Treatments and/or Uses

The present invention relates to methods for treating a subject afflicted with a condition or indication associated with IL-23 or IL-23R (e.g., activation of the IL-23/IL-23R signaling pathway), where the method comprises administering to the subject an IL-23R inhibitor disclosed herein. In one aspect, the present invention relates to a method for treating a subject afflicted with a condition or indication characterized by inappropriate, deregulated, or increased IL-23 or IL-23R activity or signaling, comprising administering to the individual a peptide inhibitor of the present invention in an amount sufficient to inhibit (partially or fully) binding of IL-23 to an IL-23R in the subject. The inhibition of IL-23 binding to IL-23R may occur in particular organs or tissues of the subject, e.g., the stomach, small intestine, large intestine/colon, intestinal mucosa, lamina propria, Peyer's Patches, mesenteric lymph nodes, or lymphatic ducts.

The present invention relates to methods comprising providing a peptide inhibitor described herein to a subject in need thereof. The subject in need thereof may be a subject that has been diagnosed with or has been determined to be at risk of developing a disease or disorder associated with IL-23/IL-23R. The subject may be a mammal. The subject may be, in particular, a human.

The disease or disorder to be treated by treatment with an IL-23R inhibitor of the present invention may be autoimmune inflammation and related diseases and disorders, such as multiple sclerosis, asthma, rheumatoid arthritis, inflammation of the gut, inflammatory bowel diseases (IBDs), juvenile IBD, adolescent IBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

The present invention relates to a method or use of an IL-23R inhibitor for treating an inflammatory disease in a subject that includes administering to the subject a therapeutically effective amount of an IL-23R inhibitor of the present invention or pharmaceutically acceptable solvate or salt thereof, or a composition disclosed herein comprising an IL-23 inhibitor of the present invention. In some aspects, the present invention provides a method of treating an inflammatory disease in a subject that includes administering to the subject a therapeutically effective amount of an IL-23R inhibitor of the present invention or pharmaceutically acceptable solvate or salt thereof, or a composition of the present invention. Suitable inflammatory diseases for treatment with a compound or pharmaceutically acceptable salt thereof, or a composition of the present invention, may include, but are not limited to inflammatory bowel disease (IBD), Crohn's disease (CD), ulcerative colitis (UC), psoriasis (PsO), or psoriatic arthritis (PsA) and the like. The inflammatory disease to be treated may be inflammatory bowel disease (IBD), Crohn's disease, or ulcerative colitis. The inflammatory disease to be treated may be selected from psoriasis, or psoriatic arthritis. The inflammatory disease to be treated may be psoriasis The inflammatory disease to be treated may be psoriatic arthritis. The inflammatory disease to be treated may be IBD.

The present invention relates to methods for treating an inflammatory disease in a subject in need thereof, comprising administering to the subject an IL-23R inhibitor disclosed herein (e.g., a peptide inhibitor or the IL-23R of Formulas (I) to (XX) or any of Tables 1A to 1H). The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula (I). The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula I. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula II. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula III. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula IV. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula V. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula VI. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula VII. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula VIII. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula IX. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula X. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XI. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XII. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XIII. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XIV. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XV. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XVI. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XVII. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XVIII. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XIX. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula XX. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods of inhibiting IL-23 binding to an IL-23R on a cell, comprising contacting the IL-23R with a peptide inhibitor of the receptor disclosed herein. The cell may be a mammalian cell. The method may be performed in vitro or in vivo. Inhibition of binding may be determined by a variety of routine experimental methods and assays known in the art.

The present invention relates to a method of selectively inhibiting IL-23 or IL-23R signaling (or the binding of IL-23 to IL-23R) in a subject (e.g., in a subject in need thereof), comprising providing to the subject a peptide inhibitor of the IL-23R described herein. The present invention includes and provides a method of selectively inhibiting IL-23 or IL-23R signaling (or the binding of IL-23 to IL-23R) in the GI tract of a subject (e.g., a subject in need thereof), comprising providing to the subject a peptide inhibitor of the IL-23R of the present invention by oral administration. The exposure of GI tissues (e.g., small intestine or colon) to the administered peptide inhibitor may be at least 10-fold, at least 20-fold, at least 50-fold, or at least 100-fold greater than the exposure (level) in the blood. In particular embodiments, the present invention includes a method of selectively inhibiting IL23 or IL23R signaling (or the binding of IL23 to IL23R) in the GI tract of a subject (e.g., a subject in need thereof), comprising providing to the subject a peptide inhibitor, wherein the peptide inhibitor does not block the interaction between IL-6 and IL-6R or antagonize the IL-12 signaling pathway. In a further related embodiment, the present invention includes a method of inhibiting GI inflammation and/or neutrophil infiltration to the GI, comprising providing to a subject in need thereof a peptide inhibitor of the present invention. In some embodiments, methods of the present invention comprise providing a peptide inhibitor of the present invention (i.e., a first therapeutic agent) to a subject (e.g., a subject in need thereof) in combination with a second therapeutic agent. In certain embodiments, the second therapeutic agent is provided to the subject before and/or simultaneously with and/or after the peptide inhibitor is administered to the subject. In particular embodiments, the second therapeutic agent is an anti-inflammatory agent. In certain embodiments, the second therapeutic agent is a non-steroidal anti-inflammatory drug, steroid, or immune modulating agent. In certain embodiments, the method comprises administering to the subject a third therapeutic agent. In certain embodiments, the second therapeutic agent is an antibody that binds IL-23 or IL-23R.

The present invention relates to methods of inhibiting IL-23 signaling by a cell, comprising contacting the IL-23R with a peptide inhibitor described herein. In certain embodiments, the cell is a mammalian cell. In particular embodiments, the method is performed in vitro or in vivo. In particular embodiments, the inhibition of IL-23 signaling may be determined by measuring changes in phospho-STAT3 levels in the cell.

In any of the foregoing methods, IL-23R inhibitor administration to a subject may be conducted orally, but other routes of administration are not excluded. Other routes of administration include, but are not limited to, parenteral, subcutaneous, intravenous, intramuscular, intraperitoneal, transdermal, topical, buccal or ocular routes. Dosages of a peptide inhibitor or the IL-23R described herein (e.g., a compound of Formula (I) to Formula (XX) or any of Tables 1A through 1H, or salt or solvate thereof to be administered to a subject may be determined by a person of skill in the art taking into account the disease or condition being treated including its severity, and factors including the age weight, sex, and the like. Exemplary dose ranges include, but are not limited to, from about 1 mg to about 1000 mg, or from about 1 mg to about 500 mg, from about 1 mg to about 100 mg, from about 10 mg to about 50 mg, from about 20 mg to about 40 mg, or from about 20 mg to about 30 mg. A dose range of a peptide inhibitor or the IL-23R described herein may be from about 600 mg to about 1000 mg. A dose range of a peptide inhibitor or the IL-23R described herein may be from about 300 mg to about 600 mg. A dose range of a peptide inhibitor or the IL-23R described herein may be from about 5 mg to about 300 mg. A dose range of a peptide inhibitor or the IL-23R described herein may be from about 25 mg to about 150 mg. A dose range of a peptide inhibitor or the IL-23R described herein may be from about 25 mg to about 100 mg. A dose range of a peptide inhibitor or the IL-23R described herein may be present in a dose range of from about 1 mg to about 100 mg. A dose range of a peptide inhibitor or the IL-23R described herein may be present in a dose range of from about 20 mg to about 40 mg. A dose range of a peptide inhibitor or the IL-23R described herein may be present in a dose range of from about 20 mg to about 30 mg.

### VII. Certain Aspects

The following aspects are illustrate the invention. These aspects are not intended to limit the scope of the present invention, but rather to provide guidance to the skilled artisan to prepare and use the compounds, compositions, and methods of the present invention. While particular aspects of the present invention are described, the skilled artisan will appreciate that various changes and modifications can be made without departing from the spirit and scope of the invention.
1. A bicyclic peptide inhibitor of an interleukin-23 receptor, which comprises an amino acid sequence of Formula I

(I)
R1-X4-X5-T-X7-X8-X9-AEF-X11-X12-
X13-N-X15-meG-R2

wherein:
   R1 is 7Ahp, 6Ahx, 5Ava, PEG2, AEEP, or AEEP(Ns);
   X4 is Pen, Abu, aMeC, hC, or C;
   X5 is N or K(PEG2PEG2gEC18OH);
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is K(Ac), Q, K(NMeAc), K(PEG2PEG2gEC180H), dK(Ac), dQ, dK(NMeAc), or dK(PEG2PEG2gEC180H);
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is THP or aMeK;
   X13 is E, dE, hE, dhR, D, dD, hSer, or dhSer;
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond or thioether bond (between pFS and Dap(pF)) between R1 and X13.
2. The bicyclic peptide inhibitor aspect 1, wherein X11 is 2Nal.
3. The bicyclic peptide inhibitor of any of aspects 1-2, wherein X7 W or 7MeW.
4. The bicyclic peptide inhibitor of any of aspects 1-3, wherein X15 is 3Pya, H, 3MeH, or F.
5. A bicyclic peptide inhibitor of an interleukin-23 receptor, which comprises an amino acid sequence of Formula II

(II)
R1-X3-X4-X5-T-X7-K(Ac)-X9-AEF-X11-
THP-X13-N-X15-X16-R2

wherein:
   R1 is GABA, pFS, bAla, or (HOC16gEPEG2PEG2)orn;
   X3 is dR, G, dK(PEG2PEG2gEC180H)R, or K(PEG2PEG2gEC180H);
   X4 is Pen, Abu, aMeC, hC, or C;
   X5 is N, or Q;
   X7 is 7MeW, or W;
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, dE, D, dD, Dap(pF(6)), or dDap(pF(6));
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedYK, dK, NMeY, NMedY, N, dH, dN, dL, Aib, L, or absent;
   X16 is meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD or is absent; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide of thioether (between pFS and Dap(pF(6)) bond between R1 and X13.
6. The bicyclic peptide inhibitor aspect 5, wherein X11 is 2Nal.
7. The bicyclic peptide inhibitor of any of aspects 5-6, wherein X15 is 3Pya or THP.
8. The bicyclic peptide inhibitor of any of aspects 5-7, wherein X16 is meG or absent.
9. A bicyclic peptide inhibitor of an interleukin-23 receptor, which comprises an amino acid sequence of Formula III

(III)
R1-X3-X4-X5-T-X7-X8-X9-X10-X11-
X12-X13-N-X15-X16-R2

wherein:
   R1 is C₁ to C₄ alkyl C(O)—, or C₁ to C₄ alkyl C(O)— substituted with Cl, F, or cyano, 5cpa, cPEG3aCO, or —H;
   X3 is R5H, S5H, R6H, S6H, R7H, S7H, K, dK, Orn, d-Orn, Dap, dDap, Dab(COCH2), dDab(COCH2), dhE, hE, hK, dhK;
   X4 is Pen, Abu, aMeC, hC, or C;
   X5 is N, Q or N(N(Me)2),
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is K(Ac), Q, K(NMeAc), dK(Ac), dQ, or dK(NMeAc);
   X9 is Pen, Abu, aMeC, hC, or C;
   X10 is AEF or TMAPF;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is THP, Acpx, or aMeK;
   X13 is R5H, R6H, R7H, S5H, S6H, S7H, C, E, hE, KNMe, dC, dE, dhE, or dKNMe;
   X15 is 3Pya, bAla, THP, dK, or aMePhe;
   X16 is meG, NMedY, or absent;
   R2 is —NH₂, N(H)(C₁-C₄ alkyl), —HN(C₁-C₄ alkyl), —N(C₁-C₄ alkyl)₂, each alkly optionally substituted with Cl, F, or cyano; and
wherein the bicyclic peptide inhibitor of an interleukin-23 receptor is cyclized by a first disulfide or thioether bond between X4 and X9, and a second amide, thioether, or aliphatic (generated from a Ring Closing Metathesis “RCM” reaction) bond between X3 and X13.
10. The bicyclic peptide inhibitor aspect 9, wherein X11 is 2Nal.
11. The bicyclic peptide inhibitor of any of aspects 9-10, wherein:
   (i) X7 is W or 7MeW; and/or
   (ii) X15 is 3Pya; and/or
   (iii) X16 is meG.
12. A bicyclic peptide inhibitor of an interleukin-23 receptor, which comprises an amino acid sequence of Formula IV

(IV)
R1-X3-X4-X5-T-X7-X8-X9-AEF-X11-
THP-X13-N-X15-X16-R2

wherein:
   R1 is C₁ to C₄ alkyl C(O)—, or C₁ to C₄ alkly C(O)— substituted with Cl, F, or cyano, —H, 7Ahp, 6Ahx, 5Ava, or GABA;
   X3 is dR, R, d-Orn, Orn, or absent;
   X4 is Pen, Abu, aMeC, hC, or C;
   X5 is Q, or N;
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is Q, K(Ac), dQ, dK(Ac);
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, aMeE, Aad, hE, K, dE, dAad, dhE, or dK;
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
   X16 is meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD or is absent; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between AEF and X13.
13. The bicyclic peptide inhibitor aspect 12, wherein X11 is 2Nal.
14. The bicyclic peptide inhibitor of any of aspects 12-13, wherein X15 is bAla, 3Pya, THP or NMedY.
15. The bicyclic peptide inhibitor of any of aspects 12-14, wherein X16 is meG or absent.
16. A bicyclic peptide inhibitor of an interleukin-23 receptor, which comprises an amino acid sequence of Formula V

(V)
R1-X4-N-T-X7-X8-X9-F4CONH2-X11-
THP-X13-N-3Pya-meG-R2

wherein:
   R1 is —H, C₁ to C₄ alkyl C(O)—, or C₁ to C₄ alkyl C(O)— substituted with Cl, F, or cyano,
   X4 is Pen, Abu, aMeC, hC, or C;
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is K or dK;
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, dE, D, dD; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between X8 and X13.
17. The bicyclic peptide inhibitor aspect 16, wherein X11 is 2Nal.
18. The bicyclic peptide inhibitor of any of aspects 16-17, wherein X7 is W or 7MeW.
19. A bicyclic peptide inhibitor of an interleukin-23 receptor, which comprises an amino acid sequence of Formula VI

(VI)
R1-X3-A-X5-T-X7-X8-A-AEF-X11-
THP-X13-N-X15-R2

wherein:
   R1 is —H, C₁ to C₄ alkyl C(O)—, or C₁ to C₄ alkyl C(O)— substituted with Cl, F, or cyano,
   X3 is K, dK, hdK, E, dE, D, dD;
   X5 is E, dE, D, dD;
   X7 is W or 7MeW;
   X8 is K(Ac) or dK(Ac);
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is K(Ac) or dK(Ac);
   X15 is K, dK, dH, hdK E, dE, D, dD; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first amide bond between X5 and X10, and a second amide bond between X3 and X15.
20. The bicyclic peptide inhibitor aspect 19, wherein X11 is 2Nal.
21. The bicyclic peptide inhibitor of any of aspects 19-20, wherein X7 is W or 7MeW.
22. The bicyclic peptide inhibitor of any of aspects 19-21, wherein X15 is K or dK.
23. A bicyclic peptide inhibitor of an interleukin-23 receptor, which comprises an amino acid sequence of Formula VII

(VII)
R1-X3-X4-N-T-X7-K(Ac)-X9-X10-
X11-THP-X13-N-3Pya-X16-R2

wherein:
   R1 is —H, C₁ to C₄ alkyl C(O)—, or C₁ to C₄ alkyl C(O)— substituted with Cl, F, or cyano,
   X3 is D, dK, E, dDap, dD, K, dE, or Dap;
   X4 is Pen, Abu, aMeC, hC, or C;
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X9 is Pen, Abu, aMeC, hC, or C;
   X10 is AEF, F4CONH2, or F40Me;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is K(Ac) or dK(Ac);
   X16 is K, dK, E, dE, R, dR, D, dD or is absent;
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between X3 and X16.
24. The bicyclic peptide inhibitor aspect 23, wherein X11 is 2Nal.
25. The bicyclic peptide inhibitor of any of aspects 23-24, wherein X7 is W or 7MeW.
26. The bicyclic peptide inhibitor of any of aspects 23-25, wherein X16 is K, dK, E, dE, R, dR, D, or dD.
27. A bicyclic peptide inhibitor of an interleukin-23 receptor, which comprises an amino acid sequence of Formula VIII

(VIII)
R1-X4-X5-X6-X7-X8-X9-X10-X11-
X12-X13-X14-3Pya-meG-R2

wherein:
   R1 is selected from —H, CF3CO, 5cpaCO, cPEG3aCO, C₁ to C₄ alkyl C(O)—, or C₁ to C₄ alkyl C(O)— substituted with cyano, Cl, F, AcMorph, or PEG12OMe;
   X4 is Pen, Abu, aMeC, hC, or C;
   X5 is selected from E, K, Dap, or K(NMe);
   X6 is selected from T, L, I;
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is selected from K(Ac), KPeg12, KAcMor, Q(N(Me)2), K(Me)3, hK(Me)3; K(NMeAc), K(mPEG12), A, or Q, dKAc, dKPeg12, dKacMor, dQ(N(Me)2), dK(Me)3, dhK(Me)3, dK(NMeAc), dK(mPEG12), dA, or dQ;
   X9 is Pen, Abu, aMeC, hC, or C;
   X10 is selected from AEF, AEF(NMe), K, or E;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is selected from THP, aMeLeu, or A;
   X13 is selected from K(Ac), A, L, K(NMeAc), Q(N(Me)2)), K(Me)3, E, dK(Ac), dA; dL, dK(NMeAc), dQ(N(Me)2)), dK(Me)3, or dE;
   X14 is selected from A, L, N or S; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide or alkyl amine (between K(NMe) and AEF) bond between X5 and X10.
28. The bicyclic peptide inhibitor aspect 27, wherein X11 is 2Nal.
29. The bicyclic peptide inhibitor of any of aspects 27-28, wherein X7 is W, 7MeW, 7PhW, dW, d7MeW, or d7PhW.
30. A bicyclic peptide inhibitor of an interleukin-23 receptor, which comprises an amino acid sequence of Formula IX

(IX)
R1-X3-X4-X5-T-X7-X8-X9-X10-X11-
THP-X13-X14-3Pya-meG-R2

wherein
   R1 is selected from —H, C₁ to C₄ alkyl C(O)—, or C₁ to C₄ alkyl C(O)— substituted with Cl; F, or cyano, or HOC18gEPEG2PEG2CO;
   X3 is R, dR, K, dK, dK(Me)3, K(Me)3, dK(PEG2PEG2gEC18OH), or K(PEG2PEG2gEC18OH);
   X4 is Pen, Abu, aMeC, hC, or C;
   X5 is selected from E;
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is selected from K(Ac) or dK(Ac);
   X9 is Pen, Abu, aMeC, hC, or C;
   X10 is selected from AEF or AEF(NMe); X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is selected from K(Ac), E, dK(Ac), or dE;
   X14 is selected from N;
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between X5 and X10.
31. The bicyclic peptide inhibitor aspect 30, wherein X11 is 2Nal.
32. The bicyclic peptide inhibitor of any of aspects 30-31, wherein X7 is W or 7MeW.
33. A bicyclic peptide inhibitor of an interleukin-23 receptor, which comprises an amino acid sequence of Formula X

(X)
X5-T-X7-X8-A-AEF-X11-
THP-X13-3Pya

wherein:
   X5 is E, dE, D, or dD;
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is K(Ac) or dK(Ac);
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy; and
   X13 is K(Ac), dK(Ac), or is absent; and
wherein the bicyclic peptide inhibitor of an interleukin-23 receptor is cyclized by a first amide bond between X5 and AEF, and a second cyclization between the amino terminus of X5 and the carboxy terminus of 3Pya.
34. The bicyclic peptide inhibitor aspect 33, wherein X11 is 2Nal.
35. The bicyclic peptide inhibitor of any of aspects 33-34, wherein X7 is W or 7MeW.
36. A bicyclic peptide inhibitor of an interleukin-23 receptor, which comprises an amino acid sequence of Formula XI

(XI)
R1-X4-X5-T-X7-X8-X9-AEF-X11-
THP-X13-N-X15-R2

wherein:
   R1 is 7Ahp, 6Ahx, 5Ava, or AEEP;
   X4 is Pen, Abu, aMeC, hC, or C;
   X5 is N, or Q,
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is K(Ac), Q, dK(Ac), or dQ;
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, dE, D, or dD;
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between R1 and X13.
37. The bicyclic peptide inhibitor aspect 36, wherein X11 is 2Nal.
38. The bicyclic peptide inhibitor of any of aspects 36-37, wherein X7 is W, 7MeW, dW, or d7MeW.
39. The bicyclic peptide inhibitor of any of aspects 36-38, wherein X15 is 3Pya THP, NMeY, or NMedY.
40. A bicyclic peptide inhibitor of an interleukin-23 receptor, which comprises an amino acid sequence of Formula XII

(XII)
R1-X4-N-X6-X7-X8-X9-AEF-2Nal-X12-X13-N-3Pya-X16-
R2

wherein:
   R1 is —H, C₁ to C₄ alkyl C(O)—, or C₁ to C₄ alkyl C(O)— substituted with Cl, F, or cyano,
   X4 is Pen, Abu, aMeC, hC, or C;
   X6 is 3Hyp, T, or 30HPro;
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is R5H, R6H, R7H, S5H, S6H, or S7H;
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is R5H, R6H, R7H, S5H, S6H, or S7H;
   X13 is K, dK, KAc, dKAc, E, dE, D, or dD;
   X16 is meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD or is absent; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide or aliphatic (generated from a Ring Closing Metathesis “RCM” reaction) bond between X8 and X12.
41. The bicyclic peptide inhibitor aspect 40, wherein X11 is 2Nal.
42. The bicyclic peptide inhibitor of any of aspects 40-41, wherein X6 is T, and/or X7 is W, 7MeW, dW, or d7MeW.
43. The bicyclic peptide inhibitor of any of aspects 36-39, wherein X16 is meG or absent.
44. A bicyclic peptide inhibitor of an interleukin-23 receptor, which comprises an amino acid sequence of Formula XIII

(XIII)
R1-X4-X5-T-X7-X8-X9-AEF-2Nal-THP-X13-N-X15-X16-
X17-R2

wherein:
   R1 is 7Ahp, 6Ahx, 5Ava, or AEEP;
   X4 is Pen, Abu, aMeC, hC, or C;
   X5 is N,
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
   X8 is K(Ac), Q, dK(Ac), or dQ;
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, dE, D, or dD;
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, NmedY, N, dH, dN, dL, Aib, L, or absent;
   X16 is meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD, NMeK(PEG2PEG2gEC180H), dNMeK(PEG2PEG2gEC180H) or is absent;
   X17 is absent, K(PEG2PEG2gEC180H), or dK(PEG2PEG2gEC180H), and
   R2 is —NH₂, N(H)(C₁-C₄ alkyl), —HN(C₁-C₄ alkyl), —N(C₁-C₄ alkyl)₂, each alkly optionally substituted with Cl, F, or cyano; and
wherein the bicyclic peptide inhibitor of an interleukin-23 receptor is cyclized by a first disulfide or thioether bond between X4 and X9, and a second amide bond between R1 and X13.
45. The bicyclic peptide inhibitor aspect 44, wherein X11 is 2Nal.
46. The bicyclic peptide inhibitor of any of aspects 44-45, wherein X7 is W, 7MeW, dW, or d7MeW.
47. The bicyclic peptide inhibitor of any of aspects 44-46, wherein X15 is 3Pya, THP, NMeY, or NMedY.
48. The bicyclic peptide inhibitor of any of aspects 44-47, wherein X16 is meG or absent.
49. A bicyclic peptide inhibitor of an interleukin-23 receptor, which comprises an amino acid sequence of Formula XIV

```
(XIV)
R1-X3-X4-X5-X6-X7-X8-X9-AEF-X11-X12-X13-N-X15-X16-R2
                                                  |
                                                  R3
```

R1 is —H, C₁ to C₄ alkyl C(O)—, or C₁ to C₄ alkly C(O)— substituted with Cl, F, or cyano,
X3 is dK, or K;
X4 is Pen, Abu, aMeC, hC, or C;
X5 is N, Q, or Dap (diamino proprionic acid also refferd to as Dpr),
X6 is T dK, or K;
X7 is W, 7MeW, dW, or d7MeW;
X8 is K(Ac), Q, dK(Ac), or dQ;
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is THP, or aMeL;
X13 is E, K(Ac), dE, E, D, dD, or dK(Ac);
X15 is 3Pya, 3MeH, H, F, hF, Y, dY, Y(CHF2), PAF, oAMPhe, F(CF3), dPaf, 3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
X16 is meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD or is absent;
R2 is —NH₂, N(H)(C₁-C₄ alkyl), —HN(C₁-C₄ alkyl), —N(C₁-C₄ alkyl)₂, each alkly optionally substituted with Cl, F, or cyano; and
R3 is PEG4 (—HN[(CH2)20]4(CH2)2CO—), PEG4DA (—OC[(CH2)20]4(CH2)2CO—), or C6-C20 saturated or unsaturated dicarboxylic acid (e.g., 1,10-decanedioic acid, 1,12-dodecanedioic acid, 1,14-tetradecanedioic acid, or 1,16-hexadecanedioic);
wherein the bicyclic peptide inhibitor of an interleukin-23 receptor is cyclized by a first disulfide or thioether bond between X4 and X9, and a second amide bond between the R3 group apended to the AEF residue at X10 and
   (i) a Dpr residue at X5,
   (ii) a K or dK at X6, or
   (iii) a K, dK, or E at X13.
50. The bicyclic peptide inhibitor aspect 49, wherein X11 is 2Nal or 3Quin or X11 is 2 Nal.
51. The bicyclic peptide inhibitor of any of aspects 49-50, wherein X7 is W, 7MeW, dW, or d7MeW.
52. The bicyclic peptide inhibitor of any of aspects 49-51, wherein X15 is H, N, dH, or dN.
53. A bicyclic peptide inhibitor of an interleukin-23 receptor, which comprises an amino acid sequence of Formula XV

(XV)
R1-X4-X5-T-X7-X8-X9-AEF-X11-THP-X13-N-X15-R2

wherein:
   R1 is —H, C₁ to C₄ alkyl C(O)—, or C₁ to C₄ alkyl C(O)— substituted with Cl, F, or cyano,
   X4 is Pen, Abu, aMeC, hC, or C;
   X5 is E, dE, D, or dD;
   X7 is W, 7MeW, dW, or d7MeW;
   X8 is K(Ac), Q, dK(Ac), or dQ;
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, K(Ac), dE, D dDor dK(Ac);
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between AEF and X5.
54. The bicyclic peptide inhibitor aspect 53, wherein X11 is 2Nal.
55. The bicyclic peptide inhibitor of any of aspects 53-54, wherein X7 is W, 7MeW, dW, or d7MeW.
56. The bicyclic peptide inhibitor of any of aspects 53-55, wherein X15 is dL, Aib, or L. A bicyclic peptide inhibitor of an interleukin-23 receptor, which comprises an amino acid sequence of Formula XVI

(XVI)
R1-X4-X5-T-X7-X8-X9-AEF-X11-THP-X13-N-X15-X16-R2

wherein:
   R1 is —H, C₁ to C₄ alkyl C(O)—, or C₁ to C₄ alkyl C(O)— substituted with Cl, F, or cyano,
   X4 is Pen, Abu, aMeC, hC, or C;
   X5 is N, or L;
   X7 is W, 7MeW, dW, or d7MeW;
   X8 is K(Ac), or dK(Ac);
   X9 is Pen, Abu, aMeC, hC, or C;
   X10 is F4CONH2, 4AmF, or F40Me;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, dK, dDap, K, Dap, dE;
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
   X16 is dK, dD, dE, Aib, G, bAla, meG, K, D, E, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, R, dR, or absent and
   R2 is absent or —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between X13 and X15, X16, R2, or the carboxy terminus of X15 or X16 when R2 is absent.
57. The bicyclic peptide inhibitor aspect 56, wherein X11 is 2Nal.
58. The bicyclic peptide inhibitor of any of aspects 56-57, wherein X7 is W, 7MeW, dW, or d7MeW.
59. The bicyclic peptide inhibitor of any of aspects 56-58, wherein X15 is dL, Aib, or L and/or X16 is dK, dD, dE, Aib, G, bAla, meG or dK.
60. A bicyclic peptide inhibitor of an interleukin-23 receptor, which comprises an amino acid sequence of Formula XVII

(XVII)
R1-X3-X4-X5-T-X7-X8-X9-X10-X11-THP-X13-X14-X15-
X16-R2

wherein:
   R1 is —H, C₁ to C₄ alkyl C(O)—, or C₁ to C₄ alkyl C(O)— substituted with Cl, F, or cyano,
   X3 is Orn, E, dOrn, or dE;
   X4 is Pen, Abu, aMeC, hC, or C;
   X5 is N;
   X7 is W, 7MeW, dW, or d7MeW;
   X8 is K(Ac), or dK(Ac);
   X9 is Pen, Abu, aMeC, hC, or C;
   X10 is F4CONH2 or AEF;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, K(Ac), dK(Ac), or dE;
   X14 is N or absent;
   X15 is 3Pya, 3MeH, H, F, hF, Y, dY, Y(CHF2), PAF, oAMPhe, F(CF3), dPaf, d3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
   X16 is 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD, dDap, meG, Dap, or is absent; and
   R2 is absent or —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide bond between X3 and one of X10, X13, or X16.
61. The bicyclic peptide inhibitor aspect 60, wherein X11 is 2Nal.
62. The bicyclic peptide inhibitor of any of aspects 60-61, wherein X7 is W, 7MeW, dW, or d7MeW.
63. The bicyclic peptide inhibitor of any of aspects 60-62, wherein X15 is 3Pya or absent. The bicyclic peptide inhibitor of any of aspects 60-63, wherein X16 is dDap, meG, Dap, or dMeG.
64. A tricyclic peptide inhibitor of an interleukin-23 receptor, which comprises an amino acid sequence of Formula XVIII

(XVIII)
R1-X3-X4-X5-T-X7-X8-X9-AEF-X11-THP-X13-N-3Pya-
meG-X17-R2

wherein:
   R1 is —H, C₁ to C₄ alkyl C(O)—, or C₁ to C₄ alkyl C(O)— substituted with Cl, F, or cyano,
   X3 is K, dK, E, dE;
   X4 is Pen, Abu, aMeC, hC, or C;
   X5 is E, dE, D, or dD;
   X7 is W or 7MeW;
   X8 is K(Ac) or dK(Ac);
   X9 is Pen, Abu, aMeC, hC, or C;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is K(Ac) or dK(Ac);
   X17 is E, dE, K, dK, D, or dD; and
   R2 is —NH₂, N(H)(C₁-C₄ alkyl), —HN(C₁-C₄ alkyl), —N(C₁-C₄ alkyl)₂, each alkyl optionally substituted with Cl, F, or cyano; and
   wherein the tricyclic peptide inhibitor of an interleukin-23 receptor is cyclized by a first disulfide or thioether bond between X4 and X9, a second amide bond between X3 and X17, and a third amide bond between X5 and AEF.
65. The bicyclic peptide inhibitor aspect 64, wherein X11 is 2Nal.
66. The bicyclic peptide inhibitor of any of aspects 64-65, wherein X7 is W or 7MeW.
67. A bicyclic peptide inhibitor of an interleukin-23 receptor, which comprises an amino acid sequence of Formula XIX

(XIX)
R1-X3-X4-X5-T-X7-X8-X9-X10-X11-X12-X13-X14-X15-
X16-X17-R2

wherein:
   R1 is 7Ahp, 6Ahx, 5Ava, PEG2, AEEP, AEEP(Ns), GABA, pFS, bAla, PEG2PEGE2gEC160H, C₁ to C₄ alkyl C(O)—, or C₁ to C₄ alkyl C(O)— substituted with Cl, F, or cyano, 5cpaCO, cPEG3aCO, or —H
   X3 is dR, R, G, R5H, R6H, R7H, S5H, S6H, S7H, K, dK, Orn, dOrn, Dap, dDap, Dab, dDab, Dab(COCH2), dDab(COCH2), hE, dhE, hK, dhK, dK(Me)3, K(Me)3, dK(PEG2PEG2gEC180H), K(PEG2PEG2gEC180H), or absent;
   X4 is Pen, Abu, aMeC, or C;
   X5 is N, Q, N(N(Me)2), or K(PEG2PEG2gEC180H);
   X7 is W, 7PhW. or 7MeW;
   X8 is K(Ac), dK(Ac), Q, dQ, K(NMeAc), dK(NmeAc), K(PEG2PEG2gEC180H), or dK(PEG2PEG2gEC180H);
   X9 is Pen, Abu, aMeC, or C;
   X10 is AEF, or TMAPF;
   X11 is 2Nal;
   X12 is THP, Acpx, or aMeK;
   X13 is E, dE, hE, dhE, aMeE, d-aMeE, D, dD, Aad, dAadK, dK, hSer, dhSer, Dap(pF), R5H, R6H, R7H, S5H, S6H, S7H, C, dC, K(NMe), or dK(NMe);
   X14 is N or absent;
   X15 is 3Pal, H, dH, 3MeH, 3MedH, F, dF, aMeF, aMedF, THP, bAla, NMeTyr, NMedY, K, dK;
   X16 is meG, NMedY, NMeK(PEG2PEG2gEC180H), NMedK(PEG2PEG2gEC180H), or absent;
   X17 is absent or K(PEG2PEG2gEC180H); and
   R2 is —NH₂, N(H)(C₁-C₄ alkyl), —HN(C₁-C₄ alkyl), —N(C₁-C₄ alkyl)₂, each alkyl optionally substituted with Cl, F, or cyano;
wherein the bicyclic peptide inhibitor of an interleukin-23 receptor is cyclized by a first disulfide or thioether bond between X4 and X9, and a second amide, aliphatic (generated from a Ring Closing Metathesis “RCM” reaction), alkyl amine, or thioether linkage between R1 and X13 or between X3 and X13.
68. A bicyclic peptide inhibitor of an interleukin-23 receptor, which comprises an amino acid sequence of Formula XX

(XX)
R1-X3-X4-X5-X6-X7-X8-X9-X10-X11-X12-X13-X14-X15-
X16-R2

wherein:
R1 is selected from CF3CO, 5cpaCO, cPEG3aCO, —H, C1 to C4 alkyl C(O)—, or C1 to C4 alkyl C(O)— substituted with cyano, Cl, or F;
X3 is R, dR, K, dK, K(Me)3, dK(Me)3, hK(Me)3, dhK(Me)3, or absent
X4 is Pen, Abu, or C;
X5 is selected from E, D, K, K(Ac), Dap, or K(NMe), K(NNs);
X6 is selected from T, L;
X7 is selected from W, 7MeW, 7PhW;
X8 is selected from K(Ac), dK(Ac), hK(Me)3, dhK(Me)3, K(Me)3, dK(Me)3, K(NMeAc), dK(NMeAc), Q(N(Me)2), KPeg12, dKPeg12, KAcMor, A, Q, dKacMor, dQ(N(Me)2), K(mPEG12), dA, dQ, or dK(mPEG12);
X9 is Pen, Abu, or C;
X10 is selected from AEF, AEF(NMe), F4CONH2, or F40Me;
X11 is 2Nal, or A;
X12 is selected from THP, aMeLeu, or A;
X13 is selected from E, dE, K(Ac), dK(Ac), K(Me)3, dK(Me)3, K(NMeAc), dK(NMeAc), Q(N(Me)2), dQ(N(Me)2), A, dA, L, or dL;
X14 is selected from L, N or S;
X15 is selected from 3Pal, L, dL, or Aib
X16 is selected from meG; and
R2 is —NH₂, N(H)(C₁-C₄ alkyl), —HN(C₁-C₄ alkyl), —N(C₁-C₄ alkyl)₂, each alkyl optionally substituted with Cl, F, or cyano; and
wherein the bicyclic peptide inhibitor of an interleukin-23 receptor is cyclized by a first disulfide or thioether bond between X4 and X9, and a second amide or alkyl amine linkage between X5 and X10.
69. The peptide inhibitor of an interleukin-23 receptor of any of aspects 1-68, wherein when: X4 is Pen, aMeC, hC, or C, and when X9 is Pen, aMeC, hC, or C, then X4 and X9 form a disulfide bond.
70. The peptide inhibitor of an interleukin-23 receptor of any of aspects 1-68, wherein when: X4 is Pen or C, and when X9 is Pen or C, then X4 and X9 form a disulfide bond.
71. The peptide inhibitor of an interleukin-23 receptor of any of aspects 1-68, wherein when: X4 is Abu and when X9 is Pen, aMeC, hC, or C, then X4 and X9 form a thioether bond.
72. The peptide inhibitor of an interleukin-23 receptor of any of aspects 1-68, wherein when: X4 is Pen, aMeC, hC, or C, and when X9 is Abu, then X4 and X9 form a thioether bond.
73. The peptide inhibitor of an interleukin-23 receptor of any of aspects 1-72, wherein X7 is W.
74. The peptide inhibitor of an interleukin-23 receptor of any of aspects 1-72, wherein X7 is 7MeW.
75. The peptide inhibitor of an interleukin-23 receptor of any of aspects 1-74, wherein X11 is 2Nal.
76. The peptide inhibitor of an interleukin-23 receptor of any of aspects 1-75, wherein when X15 is present, it is 3Pya.
77. The peptide inhibitor of an interleukin-23 receptor of any of aspects 1-76, wherein when X16 is present it is meG.
78. The peptide inhibitor of an interleukin-23 receptor of any of aspects 1-77, wherein D amino acids are present only at
   (i) one or more of positions X3, X5, X6, X8 and X13, and optionally one of positions X1-X2, X4, X7, X9 to X12, X14-X18 present in the inhibitor; or
   (ii) one or more of positions X3, X8 and X13, and optionally at one of positions X1-X2, X4-X7, X9 to X12, X14-X18 present in the inhibitor.
79. The peptide inhibitor of an interleukin-23 receptor of any of aspects 1-77, wherein D amino acids are present only at
   (i) X3, and optionally at one of positions X1-X2, X4-X18 present in the inhibitor; or
   (ii) one of positions X3, and X8, and optionally one of positions X1-X2, X4-X7, X9-X18 present in the inhibitor.
80. The peptide inhibitor of an interleukin-23 receptor of any of aspects 1-77, wherein the inhibitor comprises amino acids of the D-isomeric form at only one or two of positions X1 to X18 appearing in the IL-23R inhibitors set forth herein.
81. The peptide inhibitor of an interleukin-23 receptor of any of aspects 1-77, wherein the inhibitor comprises amino acids of the D-isomeric form at only three or four of positions X1 to X18 appearing in the IL-23R inhibitors set forth herein.
82. The peptide inhibitor of an interleukin-23 receptor of any of aspects 1-77, wherein the inhibitor comprises amino acids of the D-isomeric form at only five or six of positions X1 to X18 appearing in the IL-23R inhibitors set forth herein.
83. The peptide inhibitor of an interleukin-23 receptor, or a pharmaceutically acceptable salts, solvates, or forms thereof having a structure of a compound in Table 1A, Table 1B, Table 1C, Table 1D, Table 1E, Table 1F, Table 1G, or Table 1H.
84. The peptide inhibitor of an interleukin-23 receptor, or a pharmaceutically acceptable salts, solvates, or forms thereof having a structure of a compound in Table 1A or Table B.
85. The peptide inhibitor of an interleukin-23 receptor, or a pharmaceutically acceptable salts, solvates, or forms thereof having a structure of a compound in Table 1C or Table 1D.
86. The peptide inhibitor of an interleukin-23 receptor, or a pharmaceutically acceptable salts, solvates, or forms thereof having a structure of a compound in Table 1E or Table 1F.
87. The peptide inhibitor of an interleukin-23 receptor, or a pharmaceutically acceptable salts, solvates, or forms thereof having a structure of a compound in Table 1G or Table 1H.
88. The peptide inhibitor of an interleukin-23 receptor of any preceding aspect wherein the interleukin-23 receptor is a human interleukin receptor, for example, NCBI Reference Sequence: NP_653302.2.
89. A pharmaceutical composition which comprises:
   (i) peptide inhibitor of an interleukin-23 receptor interleukin-23 receptor or pharmaceutically acceptable salt, solvate, or form thereof according to any one of aspects 1-88, and
   (ii) a pharmaceutically acceptable carrier, excipient, or diluent.
90. A pharmaceutical composition which comprises:
   (i) peptide inhibitor of an interleukin-23 receptor interleukin-23 receptor or pharmaceutically acceptable salt, solvate, or form thereof according to any one of aspects 1-79, and
   (ii) a pharmaceutically acceptable carrier, excipient, or diluent.
91. A pharmaceutical composition which comprises:
   (i) peptide inhibitor of an interleukin-23 receptor interleukin-23 receptor or pharmaceutically acceptable salt, solvate, or form thereof according to aspect 80 or 81; and
   (ii) a pharmaceutically acceptable carrier, excipient, or diluent.
92. A pharmaceutical composition which comprises:
   (i) peptide inhibitor of an interleukin-23 receptor interleukin-23 receptor or pharmaceutically acceptable salt, solvate, or form thereof according to aspect 82 or 83; and
   (ii) a pharmaceutically acceptable carrier, excipient, or diluent.
93. A pharmaceutical composition which comprises:
   (i) peptide inhibitor of an interleukin-23 receptor interleukin-23 receptor or pharmaceutically acceptable salt, solvate, or form thereof according to aspect 84 or 85; and
   (ii) a pharmaceutically acceptable carrier, excipient, or diluent.
94. A pharmaceutical composition which comprises:
   (i) peptide inhibitor of an interleukin-23 receptor interleukin-23 receptor or pharmaceutically acceptable salt, solvate, or form thereof according to aspect 86 or 87; and
   (ii) a pharmaceutically acceptable carrier, excipient, or diluent.
95. The use of a peptide inhibitor of an interleukin-23 receptor according to any of aspects 1-88 for the preparation of a medicament.
96. The use of a peptide inhibitor of an interleukin-23 receptor according to any of aspects 1-88, or a pharmaceutical composition according to any of aspects 89-94, for the preparation of a medicament for the treatment of an inflammatory disorder or autoimmune inflammatory disorder.
97. The use of claim 18, for the preparation of a medicament for the treatment autoimmune inflammation and related diseases and disorders including, but not limited to: multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.
98. The use of aspect 97, wherein the diseases or disorders are selected from Inflammatory Bowel Disease (IBD), Ulcerative colitis (UC), Crohn's Disease (CD), psoriasis (PsO) or psoriatic arthritis (PsA).
99. A method for treating a disease or disorder associated with Interleukin 23 (IL-23)/Interleukin 23 Receptor (IL-23R), which comprises administering:
   (i) an effective amount of a peptide inhibitor of an interleukin-23 receptor, or a pharmaceutically acceptable salt, solvate, or form thereof according to any one of aspects 1-88; or
   (ii) a pharmaceutical composition according to any one of aspects 89 to 94, respectively to a patient in need thereof.
100. The method of aspect 99, wherein the disease or disorder is associated with autoimmune inflammation.
101. The method of aspect 99, wherein the disease or disorder is associated with multiple sclerosis, asthma, rheumatoid arthritis, inflammation of the gut, inflammatory bowel diseases (IBDs), juvenile IBD, adolescent IBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease
102. The method of aspect 99, wherein the disease or disorder is associated with Ulcerative colitis (UC), Crohn's Disease (CD), psoriasis (PsO), or psoriatic arthritis (PsA).
103. The method of aspect 99, wherein the disease or disorder is Ulcerative colitis (UC).
104. The method of aspect 99, wherein the disease or disorder is Crohn's Disease (CD).
105. The method of aspect 99, wherein the disease or disorder is psoriasis (PsO).
106. The method of aspect 99, wherein the disease or disorder is psoriasis psoriatic arthritis (PsA).
107. A kit which comprises a peptide inhibitor of an interleukin-23 receptor of an of aspects 1-88, or a pharmaceutical composition according to any of aspects 89-94, and instructions for the use of the inhibitor of an interleukin-23 receptor or pharmaceutical composition.
108. The kit of aspect 107, wherein the instructions are directed to the treatment of an inflammatory disease or disorder.
109. The kit of aspect 108, wherein the disease is inflammatory bowel disease (IBD), Crohn's disease (CD), ulcerative colitis (UC), psoriasis (PsO), and psoriatic arthritis (PsA).
110. Methods of preparing a compound any of aspects 1-88, comprising linking one or more monomers and causing the the formation of a first bond and a second bond resulting in a bicyclic structure.
111. A bicyclic peptide inhibitor of an interleukin-23 receptor, comprising an amino acid sequence of Formula XXI

(A)
R1-X4-X5-T-X7-X8-X9-AEF-X11-X12-X13-N-X15-meG-R2

wherein:
   R1 is 7Ahp, 6Ahx, 5Ava, Peg2, AEEP, or AEEP(Ns);
   X4 is Pen, Abu, aMeC, hC, or C;
   X5 is N or K(PEG2PEG2gEC180H);
   X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(6(2OxdeQuin8Me))W, 7(6(2OxIquin))W, 7(7(124TAZP))W, 7(7(2OMeQuin))W, 7(7Imzpy)W, 7BrW, 7EtW, 7PhW, 7PyrW, A, BT, D7MeW, or Trp sub at position 7 with C1 to C7 alkyl, halo, haloalkyl, OH, CN, C1 to C7 alkoxy, 5 to 7 membered heteroaryl containing 1 to 2 nitrogen, and/or sulfur, and/or oxygen.
   X8 is K—Ac, Q, K(NMeAc), K(PEG2PEG2gEC180H), dK-Ac, dQ, dK(NMeAc), or dK(PEG2PEG2gEC180H);
   X9 is Pen, Abu, aMeC, hC, or C;
   X12 is THP, aMeK, Aib, Acpx, Achx, 4diFAchx, aMeL, Pip(NMe), Pip(NMe2), or alpha, alpha-disubstituted (C1-C5 alkyl and C1 to C5 alkyl, haloalkyl, alkoxy, carboxy, alkylamine, or 3-7 membered carbocycle or heterocycle containing 1 to 2 nitrogen, sulfur and/or oxygen) glycine.
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy, and their heteroaromatics analogs containing 1 to 2 nitrogen, sulfur and/or oxygen.
   X13 is E, dE, hE, dhE, D, dD, or hSer, dhSer;
   X15 is 3pya, 3MeH, H, F, hF, Y, dY, Y(CHF2), PAF, oAMPhe, F(CF3), dPaf, D3Pya, ACIPA(SR), 6OH3Pya, SPyrimidAla, SMePyridinAla, 5MeH, SAmPyridinAla, 4TriazolAla, 4PyridinAla, 4Pya, 3QuinolAla, 3OHPhe, 3AmPyrazolAla, 2AmTyr, 1MeH, THP, bAla, NmeDTyr, K, dK, NMeY, NmedY, N, dH, dN, dL, Aib, L, 3pya sub with C1 to C7 alkyl, halo, haloalkyl, OH, CN, C1 to C7 alkoxy, H sub with C1 to C7 alkyl, halo, haloalkyl, OH, CN, C1 to C7 alkoxy, 5-7 membered heteroaromatics containing 1 to 2 nitrogen, sulfur and/or oxygen, 5-7 membered heteroaromatics containing 1 to 2 nitrogen, sulfur and/or oxygen and substituted with C1 to C7 alkyl, halo, haloalkyl, OH, CN, C1 to C7 alkoxy or absent;
   R2 is —NH2, N(H)C₁ to C₄ alklyl, —H(C₁-C₄ alkyl, —N(C₁ to C₄ alkyl)2, each alkyl optionally substituted with Cl, F, or cyano; and
wherein the bicyclic peptide inhibitor of an interleukin-23 receptor is cyclized by a first disulfide or thioether bond between X4 and X9, and a second amide bond or thioether bond (between pFS and Dap(pF)) between R1 and X13, or any aliphatic linker composition with a combined length of 10-18 covalent bonds, or any aliphatic and/or aromatic linker composition with equivalent length between alpha-carbons of X4 and X13.
112. A pharmaceutical composition comprising an inhibitor of an interleukin-23 receptor of aspect 111, and at a pharmaceutically acceptable excipient.
113. A method of treatment comprising administering an inhibitor of an interleukin-23 receptor of aspect 111 or a pharmaceutical composition of aspect 112 to a patient in need thereof

Some abbreviations useful in describing the invention are defined below in the following Table 2A to 2D.

TABLE 2A
Amino Acid Abbreviations

| Abbreviation | Definition | Smiles |
|---|---|---|
| dR, arg, or r | D-Arginine | |
| dK, (D)Lys, | D-lysine | |
| (D)-Lys, lys, or | | |
| k | | |
| 5Apa | 5AminoPentanoicAcid | |
| 2-Nal or 2Nal | 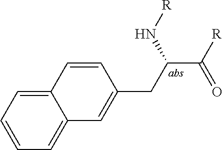 | O═C([C@H](Cc 1cc2ccccc2cc1) N[R])[R] |
| | C₁₃H₁₁NOR₂ | |
| 3MeH | 3-methyl-L-histidine | Cn1cncc1C[C@ |
| | | H](N[R])C([R])═O |
| 3Pya, 3Pal, 3- (2-pyridyl)- alanine | 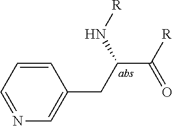 | O═C([C@H](Cc 1cnccc1)N[R])[R] |
| THP, 4- aminotetrahydro- 2H-pyran-4- carboxylic acid | 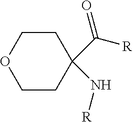 | O═C(C1(CCOC C1)N[R])[R] |
| | 4-amino-4-carboxy- | |
| | tetrahydropyran | |
| 7PhW, 7PhTrp or W(7-Ph) | 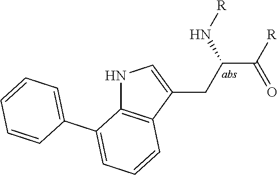 | O═C([C@H](Cc 1c[nH]c2c1cccc 2- c1ccccc1)N[R]) [R] |
| | 7-phenyl-L-tryptophan | |
| 7MeW, 7(MeW), 7MeTrp, 7- methyl-L- tryptophan | 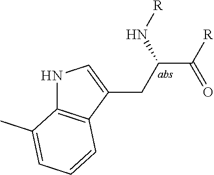 | Cc1cccc2c1[nH] cc2C[C@@H] (C([R])═O)N[R] |
| | 7-methyl-L-tryptophan | |
| Abu | 2-aminobutyric acid | C[C@@H](C═ |
| | | O)N |
| AEF, Phe(4-(2- aminoethoxy)), or F(4-2ae) | 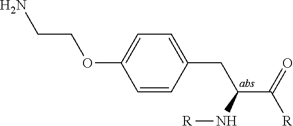 | NCCOc1ccc(C[ C@@H](C([R])═ O)N[R])cc1 |
| | 4-(2-aminoethoxy)-L- | |
| | phenylalanine | |
| Ahp, 7Ahp, | 7-aminoheptanoic acid | O═C([R])CCCC |
| 7AHP, or | | CCN[R] |
| 7 AHP(2) | | |
| Ahx or 6Ahx, | 6-aminohexanoic acid | O═C(CCCCCN |
| 6Ahx, | | [R])[R] |
| 6Ahx(2), | | |
| 6- | | |
| aminohexanoic | | |
| acid | | |
| aMeF, aMePhe, or aMe-Phe | 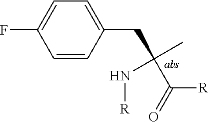 | C[C@](Cc(cc1) ccc1F)(C([R])═ O)N[R] |
| | alpha-methyl L-phenylalanine | |
| aMeK, | alpha-methyl L-lysine | |
| aMeLys, or | | |
| aMe-Lys | | |
| Arg or R | L-arginine | |
| dR, arg, r or | D-arginine | |
| (D)Arg | | |
| Asn or N | L-asparagine | |
| Ava, 5Ava(2), or 5Ava | 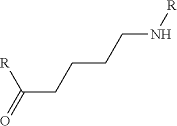 | O═C(CCCCN[R])[R] |
| | 5-Aminovaleric Acid | |
| bAla, b-ALA, beta-Alanine, bA | 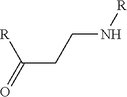 | O═C(CCN[R])[R] |
| | beta-alanine | |
| Bis-amino- | 1,2-bis(2-aminoethoxy)ethane | |
| PEG2 | | |
| Cys or C | L-cysteine | |
| Dbu, Dab, (S)- 2,4- diaminobutanoic acid, or DAB | 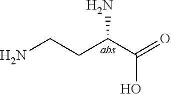 | NCC[C@@H] (C(O)═O)N |
| | L-2,4-diaminobutyric acid | |
| Dap, Dap, DAP, Dpr or (S)-2,3- diaminopropanoic acid | 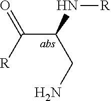 | NC[C@@H] (C([R])═O)N[R] |
| | L-2,3-diaminopropionic acid | |
| dDab, D(Dab), | D-2,4-diaminobutyric acid | NC[C@H](C([R])═ |
| dDpr, (R)-2,3- | | O)N[R] |
| diaminopropanoic | | |
| acid | | |
| dDap, D(Dap), dDap, dap, dDbu, (R)-2,3- diaminopropanoic acid | 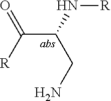 | NC[C@H](C([R])═ O)N[R] |
| | D-2,3-diaminopropionic acid | |
| Fmoc-2Nal | 2-((((9H-fluoren-9- | |
| | yl)methoxy)carbonyl)amino)-3- | |
| | (naphthalen-2-yl)propanoic acid | |
| Fmoc-3Pya | (S)-2-((((9H-fluoren-9- | |
| | yl)methoxy)carbonyl)amino)-4- | |
| | (pyridin-3-yl)butanoic acid | |
| Fmoc-7MeW | (S)-2-((((9H-fluoren-9- | |
| | yl)methoxy)carbonyl)amino)-3-(7- | |
| | methyl-1H-indol-3-yl)propanoic acid | |
| Fmoc-AEF | (S)-2-((((9H-fluoren-9- | |
| | yl)methoxy)carbonyl)amino)-3-(4-(2- | |
| | ((tert- | |
| | butoxycarbonyl)amino)ethoxy)phenyl) | |
| | propanoic acid | |
| Fmoc-aMePhe | (((9H-fluoren-9- | |
| | yl)methoxy)carbonyl)-alphamethyl-L- | |
| | phenylalanine | |
| Fmoc-arg or Fmoc-r | N-alpha-(9- | |
| | fluorenylmethyloxycarbonyl)-N′- | |
| | 2,2,4,6,7- | |
| | pentamethyldihydrobenzofuran-5- | |
| | sulfonyl-D-arginine | |
| Fmoc-Asn or Fmoc-N | N₂-(((9H-fluoren-9- | |
| | yl)methoxy)carbonyl)-N₄-trityl-L- | |
| | asparagine | |
| Fmoc-Dap(DDe) | N2-(Fmoc)-N6-(1-(4,4-dimethyl-3,5- | |
| | dioxocyclohexylidene)ethyl)-L-Dap | |
| Fmoc-DDe- | N6-(((9H-fluoren-9- | |
| Lys(Fmoc)═OH | yl)methoxy)carbonyl)-N₂-(1-(4,4- | |
| | dimethyl-3,5- | |
| | dioxocyclohexylidene)ethyl)-L-lysine | |
| Fmoc-Glu or Fmoc-E | (S)-2-((((9H-fluoren-9- | |
| | yl)methoxy)carbonyl)amino)-5-(tert- | |
| | butoxy)-2-methyl-5-oxopentanoic | |
| | acid | |
| Fmoc-Lys(Ac) or | N₂-(((9H-fluoren-9- | |
| Fmoc-K(Ac) | yl)methoxy)carbonyl)-N6-acetyl-L- | |
| | lysine | |
| Fmoc-Lys(DDe) or | N2-(Fmoc)-N6-(1-(4,4-dimethyl-3,5- | |
| Fmoc-K(DDe) | dioxocyclohexylidene)ethyl)-L-lysine | |
| Fmoc-Lys(NMeAc) or | N₂-(((9H-fluoren-9- | |
| Fmoc-K(NMeAc) | yl)methoxy)carbonyl)-N6-acetyl-N6- | |
| | methyl-L-lysine | |
| Fmoc-NMeLys(DDe) | (9H-fluoren-9-yl)methyl (1- | |
| or Fmoc-NMeK(DDe) | amino-6-((1-(4,4-dimethyl-3,5- | |
| | dioxocyclohexylidene)ethyl)amino)- | |
| | 1-oxohexan-2- | |
| | yl)(methyl)carbamate | |
| Fmoc-Pen-Trt | (R)-2-((((9H-fluoren-9- | |
| | yl)methoxy)carbonyl)amino)-3- | |
| | methyl-3-(tritylthio)butanoic acid | |
| Fmoc-Pro or | Fmoc-proline-OH | |
| Fmoc-P | | |
| Fmoc-pro or | Fmoc-D-proline-OH | |
| Fmoc-p | | |
| Fmoc-R5H | (R)-2-((((9H-fluoren-9- | |
| | yl)methoxy)carbonyl)amino)hept- | |
| | 6-enoic acid | |
| Fmoc-Sar or Fmoc- | N-(((9H-fluoren-9- | |
| Sarc | yl)methoxy)carbonyl)-N- | |
| | methylglycine | |
| Fmoc-THP | 4-((((9H-fluoren-9- | |
| | yl)methoxy)carbonyl)amino)tetrahydro- | |
| | 2H-pyran-4-carboxylic acid | |
| Fmoc-Thr or Fmoc-T | N-(((9H-fluoren-9- | |
| | yl)methoxy)carbonyl)-O-(tert-butyl)- | |
| | L-threonine | |
| GABA, Gaba, Gaba(2), Gaba2, or 4Abu | 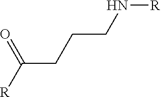 | O═C(CCCN[R])[R] |
| | 4-aminobutyric acid | |
| Glu or E | L-glutamic acid | |
| glu or e or D(Glu) | D-glutamic acid | |
| His or H | L-histidine | |
| Lys or K | L-lysine | |
| lys or k or | D-lysine | |
| (D)Lys | | |
| hCys, hC | L-Homocysteine | C(CS)[C@@H](C |
| | | (═O)O)N |
| KAc, Lys(Ac), | N-ε-acetyl-L-Lysine | CC(NCCCC[C |
| K(Ac), | N6-Acetyl-L-lysine | O)N[R])═O |
| K(COMe), or | | |
| K-Ac | | |
| MeK, N- | N-methyl-Lysine | |
| MeLys, | (2S)-2-amino-6- | |
| NMeLys, | (methylamino)hexanoic acid | |
| NMeK, or | | |
| MeLys | | |
| Pen | 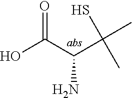 | CC(C)([C@@H] (C(O)═O)N)S |
| | L-penicillamine, 3-Mercapto-L- | |
| | valine | |
| | (R)-2-Amino-3-mercapto-3- | |
| | methylbutanoic acid | |
| F₄CONH2, | 4-carbamoyl-L-phenylalanine | N[C@H](C([R]═O) |
| Phe(4-CONH₂) | (S)-2-amino-3-(4- | Cc1ccc(C(N |
| or Phe(4- | carbamoylphenyl)propanoic acid | [R])═O)cc1 |
| CONH2) or | | |
| Phe(Cmd) or | | |
| Phe_4Ad | | |
| F₄OMe, | 4-methoxy-L-phenylalanine | N[C@@H](CC1═ |
| Phe(4═OMe), | | CC═C(OC)C═ |
| or Phe_4OMe | | C1)C(O)═O |
| Quin, 3Quin, 3-Quin, 3QuinolAla, or 3QuinA | 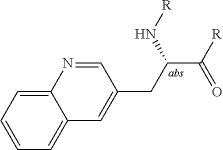 | O═C([C@H](Cc 1cc2ccccc2nc1) N[R])[R] |
| | (S)-2-amino-3-(quinolin-3- | |
| | yl)propanoic acid | |
| R5H, | (R)-2-aminopentanoic acid 5-diyl | |
| R6H, (R,E)-2- | (R)-2-aminohexanoic acid 6-diyl | C═CCCCC[C@ |
| amino-8- | | H](C([R])═O)N[R] |
| hydroxyoct-7- | | |
| enoic acid | | |
| R7H, (R,E)-2- | (R)-2-aminoheptanoic acid 7-diyl | C═CCCCCC[C |
| amino-9- | | @H](C([R])═O) |
| hydroxynon-8- | | N[R] |
| enoic acid | | |
| S5H | (S)-2-aminopentanoic acid 5-diyl | C═CCCC[C@H] |
| | | (N[R])C([R])═O |
| meG, Sarc, MeGly, Sar, Sarc, MeGly, Sarcosine, Methylamino- Acetic Acid,N- methylglycine | 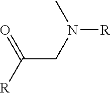 sarcosine or N-methylglycine | CN(CC([R])═O) [R] |
| Thr or T | L-threonine | |
| nFEtOH, Phe(4- | Fc1c(F)c([H])c(F)c(F)c1NC[C@@H] | N[C@@H](C═O)c |
| OCH2COOH, or 2- | (C([R])═O)N[R] | (cc1)ccc1OCC═O |
| amino-2-[4- | (R)-2-amino-2-(4- | |
| (carboxymethoxy) | (carboxymethoxy)phenyl)acetic acid | |
| phenyl]acetic acid, | | |
| DappF6 | tetra-fluoro-phenylalanine | Fc1c(F)c([H])c(F)c |
| Dap(pF(6)) | | (F)c1NC[C@@H](C |
| | | ([R])═O)N[R] |

TABLE 2B
Abbreviations for Substituents, Reagents, and Solvents

| Abbreviation | Definition |
|---|---|
| Ac or MeCO | acetyl |
| ACN | acetonitrile |
| Boc | tert-butoxy-carbonyl |
| CONH₂ | carboxamide |
| COOH | carboxylic Acid |
| DCM | dichloromethane |
| Dde | N-(1-(4,4-dimethyl-2,6-dioxocyclohexylidene)ethyl |
| DIC | N,N′-diisopropylcarbodiimide |
| DMF | N,N-dimethylformamide |
| Et₂O | di-ethylether |
| FMOC or Fmoc | ((9H-fluoren-9-yl)methoxy)carbonyl |
| HOAT or HOAt | 1-hydroxy-7-azabenzotriazole |
| MeOH | methanol |
| MTBE | methyl tert-butyl ether |
| MW | microwave |
| Oxyma | ethyl cyanohydroxyiminoacetate |
| PEG2 DiAcid or PEG2DA | 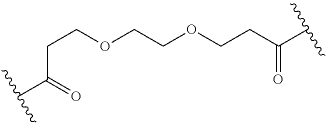 [SMILES: *C(=O)CCOCCOCCC(*)=O] |
| | 3,3′-(ethane-1,2-diylbis(oxy))dipropanecarbonyl |
| pF | 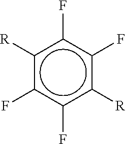 |
| | 2,3,5,6-tetrafluorophen-1,4-diyl linker |
| | Fc1c(F)c([R])c(F)c(F)c1[R] |
| pFS | 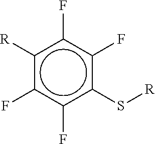 |
| | 2,3,5,6-tetrafluoro-4-mercaptophenol |
| | Fc(c(S[R])c(c(F)c1[R])F)c1F |
| RT | room temperature |
| TFA | trifluoroacetic acid |
| TIPS | triisopropylsilane |

TABLE 2C
Monomers and structures

| # | Symbol/Name | Structure | Smiles |
|---|---|---|---|
| 1 | bMeW(2S3R) bMeW(2S,3R) | 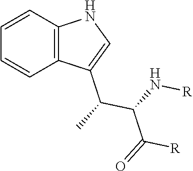 | C[C@H](C1═CN C₂═C1C-CC═C₂) [C@H](N[R])C ([R])═O |
| | | C₁₂H₁₂N₂OR₂ | |
| 2 | bMeW(2S3S), bMeW(2S,3S) | 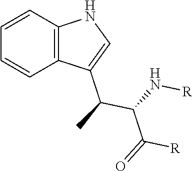 | C[C@@H](C1═C NC₂═C1C═CC═C 2)[C@H](N[R]) C([R])═O |
| | | C₁₂H₁₂N₂OR₂ | |
| 3 | 6OH2Nal | 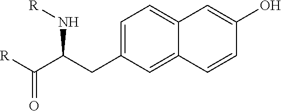 | [R]C([C@H](CC1═ CC═C(C═C(O)C═ C₂)C₂═C1)N[R])═ O |
| | | (S)-2-amino-3-(6-hydroxynaphthalen-2-yl)propanoic acid | |
| 4 | NMe7MeW | 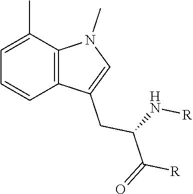 | [R]C([C@@H](N[R]) CC1═CN(C)C2═ C1C═CC═C2C)═O |
| | | C₁₃H₁₄N₂OR₂ | |
| 5 | 7(4Paz)W | 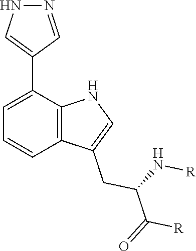 | [R]C([C@@H](N [R])CC1═CNC2═C 1C═CC═C2C3═C NN═C3)═O |
| | | C₁₄H₁₂N₄OR₂ | |
| 6 | 7(7(124TAZP))W | 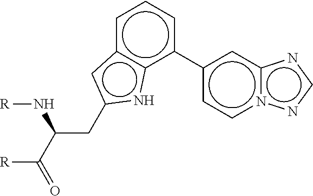 | O═C([R])[C@@H] (N[R])Cc1[nH]c2 c(c(c3)ccn4c3ncn4) cccc2c1 |
| | | C₁₇H₁₃N₅OR₂ | |
| 7 | 7(3UrPh)W | 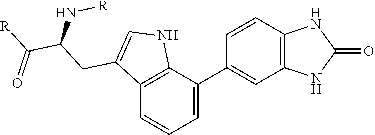 | [R]C([C@@H](N [R])CC1═CNC2═C 1C═CC═C2C3═C C(NC(N4)═O)═C4 C═C3)═O |
| | | C₁₈H₁₄N₄O₂R₂ | |
| 8 | 7(7Imzpy)W | C₁₈H₁₄N₄OR₂ | [R]C([C@@H](N |
| | | | [R])CC1═CNC2═C |
| | | | 1C═CC═C2C3═C |
| | | | C4═NC═CN4C═C |
| | | | 3)═O] |
| 9 | 7(4OMePh)W | 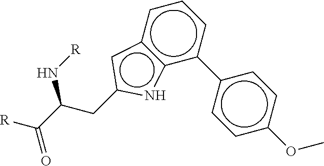 | [R]C([C@@H](N R])CC1═CNC2═C 1C═CC═C2C3═C C═C(OC)C═C3)═ O |
| | | C₁₈H₁₆N₂O₂R₂ | |
| 10 | 7(3(6AzaIndlMe))W | 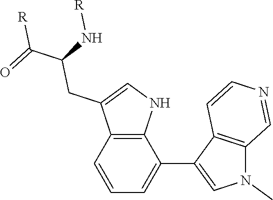 | [R]C([C@@H](N [R])CC1═CNC2═C 1C═CC═C2C3═C N(C)C4═C3C═ CN═C4)═O |

C₁₉H₁₆N₄OR₂

| | | | |
|---|---|---|---|
| 11 | 7(6(2MeNDAZ))W | 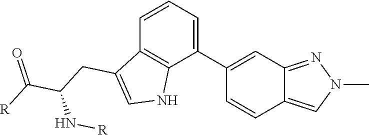 | [R]C([C@@H](N [R])CC1═CNC2═C 1C═CC═C2C3═C C4═NN(C)C═ C4C═C3)═O |

C₁₉H₁₆N₄OR₂

| | | | |
|---|---|---|---|
| 12 | NMebAla | 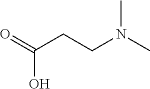 | CN(C)CCC═O |

C₅H₁₁NO

| | | | |
|---|---|---|---|
| 13 | AcMorp, Ethyl- morpholino | 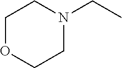 | CN1CCOCC1 |

C₅H₁₁NO

| | | | |
|---|---|---|---|
| 14 | dOrn, D-Orn D-Ornithine | 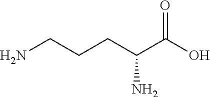 | NCCC[C@H](C (O)═O)N |

C₅H₁₂N₂O

| | | | |
|---|---|---|---|
| 15 | 3Hyp, 3-Hydroxy-L- proline | 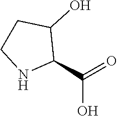 | OC1[C@@H](C═ O)NCC1 |

C₅H₉NO₂

| | | | |
|---|---|---|---|
| 16 | aMeE aMeGlu, alpha- methyl glutamic acid | 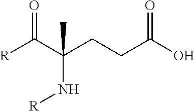 | C[C@](CCC(O)═O) (C([R])═O)N[R] c[c@](CCC(O)═O) (C═O)N |

C₆H₁₁NO₂

| | | | |
|---|---|---|---|
| 17 | hGlu, (S)-2- aminohexanedioic acid | 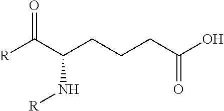 | N[C@@H](CCC C═O)C═O OC(CCC[C@@H] (C([R])═O)N[R])═O |

C₆H₁₁NO₂

| | | | |
|---|---|---|---|
| 18 | CON(NMePip) | 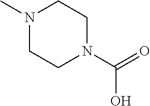 | CN(CC1)CCN1C═O |

C₆H₁₂N₂O

| | | | |
|---|---|---|---|
| 19 | -CODiFPip, CO(DiFPip) | 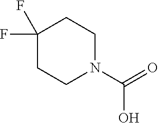 [SMILES: O=C(O)N1CCC(F)(F)CC1] | O═CN(CC1)CCC1 (F)F |

C₆H₉F₂NO

| | | | |
|---|---|---|---|
| 20 | CO(OAZBO) | 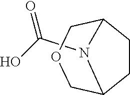 [SMILES: O=C(O)N1C2CCC1COC2] | CC(N1C2COCC1C C2)═O |

C₈H₁₃NO₂

| | | | |
|---|---|---|---|
| 21 | Me1Pya, (S)-3-(2- amino-2- carboxyethyl)-1- methylpyridin-1-ium | 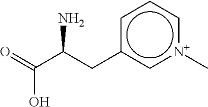 | C[n+]1cccc(C[C@ @H](C═O)N)c1 |

C₉H₁₃N₂O⁺

| | | | |
|---|---|---|---|
| 22 | DappF6, tetra-fluoro- phenylalanine | 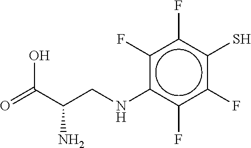 | N[C@@H](CNc(c (F)c(c(S)c1F)F)c1F) C═O |

C₉H₈F₄N₂O

| | | | |
|---|---|---|---|
| 23 | bMePhe(2S,3R) bMePhe(SR), bMePhe(2S,3R) | 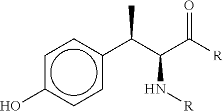 | C[C@@H]([C@ @H\|(C([R])═O)N[ R])c1ccccc1 |

C₁₀H₁₁NOR₂

| | | | |
|---|---|---|---|
| 24 | N4AmBenzylGly, N(4AmBenzyl)Gly | 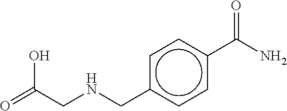 | NC(c1ccc(CNCC═ O)cc1)═O |

C₁₀H₁₂N₂O₂

| | | | |
|---|---|---|---|
| 25 | -Dec, 1,10- Decanedioic Acid | 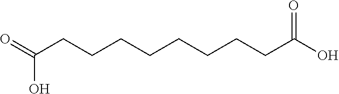 [SMILES: O=C(O)CCCCCCCCC(=O)O] | OC(CCCCCCCCC═ O)═O |

C₁₀H₁₈O₃

| | | | |
|---|---|---|---|
| 26 | 2OH3Pyrimid5 Ala | 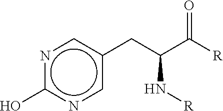 | CC(C)(C)Oc1ncc (C[c@@H](C([R])═ O)N[R])cn1 |

C₁₁H₁₅N₃O₂R₂

| | | | |
|---|---|---|---|
| 27 | KacMorph, K(AcMorph), KAcMorph, L- Lysine(ac- Morpholino | 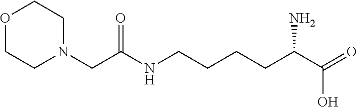 [SMILES: N[C@@H](CCCCNC(=O)CN1CCOCC1)C(=O)O] | N[C@@H](CCCC NC(CN1CCOCC1)═ O)C═O |

C₁₂H₂₃N₃O₃

| | | | |
|---|---|---|---|
| 28 | 6OH2Nal | 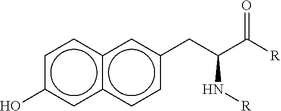 | N[C@@H](Cc1cc 2ccccc2cc1)C═O N[C@@H](Cc1cc 2ccccc2cc1)C═O Oc1ccc(cc(C[C@ @H](C([R])═O)N [R])cc2)c2c1 |

C₁₃H₁₃NO

| | | | |
|---|---|---|---|
| 29 | DabNMecarn, Dab(NMecarn) | 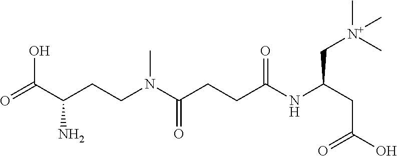 C₁₆H₃₁N₄O₄⁺ | CN(CC[C@@H]( C═O)N)C(CCC(N [C@H](CC═O)C[N+] (C)(C)C)═O)═O CN(CC[C@@H](C═ O)N)C(CCC(N[ C@H](CC═O)C[N] (C)(C)C)═O)═O CN(CC[C@@H](C═ O)N)C(CCC(N [C@H](CC(O)═O) C[N+](C) |
| | | | (C)C)═O)═O |

| | | | |
|---|---|---|---|
| 30 | DabNMeCarn, Dab(NMeCarn) | 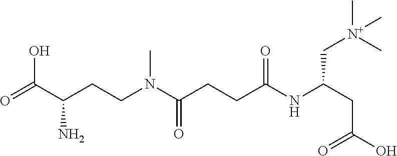 | CN(CC[C@@H]( C═O)N)C(CCC(N[ C@@H](CC(O)═ O)C[N+](C)(C)C)═ O)═O |

C₁₆H₃₁N₄O₅⁺

| | | | |
|---|---|---|---|
| 31 | F(4TzlTMA4) | 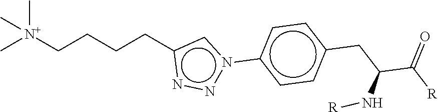 | C[N+](C)(C)CCC Cc1cn(- c2ccc(C[C@@H] (C([R])═O)N[R])cc 2)nn1 |

C₁₈H₂₆N₅OR₂⁺

| | | | |
|---|---|---|---|
| 32 | NMeK(d), NMeKdCar | 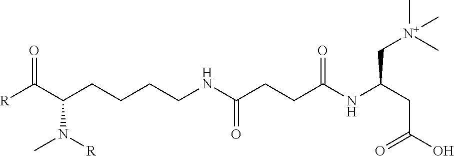 | CN([C@@H](CC CCNC(CCC(N[C @H](CC(O)═O)C [N+](C)(C)C)═O)═ O)C([R])═O)[R] |

| | | | |
|---|---|---|---|
| | | C₁₈H₃₃N₄O₅R₂⁺ | |
| 33 | 7(5(Ina7Pyr))W | C₁₉H₁₈N₄OR₂ | [R]C([C@@H](N |
| | | | [R])CC1═CNC2═C |
| | | | 1C═CC═C2C3═C |
| | | | N═C(N(C)CC4)C4═ |
| | | | C3)═O |
| 34 | F(4TzlTMA5) | C₁₉H₂₈N₅OR₂⁺ | C[N+](C)(C)CCC |
| | | | CCc1cn(- |
| | | | c2ccc(C[C@@H] |
| | | | (C([R])═O)N[R])cc |
| | | | 2)nn1 |

| | | | |
|---|---|---|---|
| 35 | CF3CO F3CO | 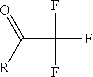 | O═C(C(F)(F)F)[R] |

C₂F₃OR

| | | | |
|---|---|---|---|
| 36 | CF3Propylamide | 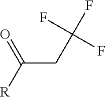 | O═C(CC(F)(F)F)[R] |

C₃H₂F₃OR

| | | | |
|---|---|---|---|
| 37 | C(1*) (*pure but configuration unknown) | 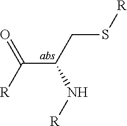 | O═C([C@H](CS[R]) N[R])[R] |

C₃H₄NOSR₃

| | | | |
|---|---|---|---|
| 38 | bAla, b-ALA, beta- Alanine, bA | 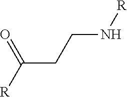 | O═C(CCN[R])[R] |

C₃H₅NOR₂

| | | | |
|---|---|---|---|
| 39 | CON(Me)2 | 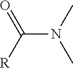 | CN(C)C([R])═O |

C₃H₆NOR

| | | | |
|---|---|---|---|
| 40 | D(2) | 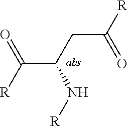 | O═C(C[C@@H](C ([R])═O)N[R])[R] |

C₄H₄NO₂R₃

| | | | |
|---|---|---|---|
| 41 | cPrCO | 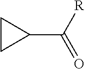 | O═C(C1CC1)[R] |

C₄H₅OR

| | | | |
|---|---|---|---|
| 42 | hS, hS, , Hse, L- homoserine, homoS, or homoSer | 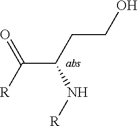 | OCC[C@@H](C ([R])═O)N[R] |

C₄H₇NO₂R₂

| | | | |
|---|---|---|---|
| 43 | T, dThr, dT | 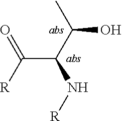 | C[C@H]([C@H](C ([R])═O)N[R])O |

C₄H₇NO₂R₂

| | | | |
|---|---|---|---|
| 44 | 4sb, 4SB | 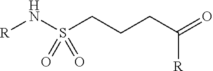 | O═C([R])CCCS(═O) (N[R])═O |

C₄H₇NO₃SR₂

| | | | |
|---|---|---|---|
| 45 | Aib, AIB, 2- Aminoisobutyric acid, Alpha- aminoisobutyric acid, (2-aminoalanine) | 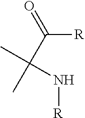 | CC(C)(C([R])═O)N [R] |

| | |
|---|---|
| | C₄H-NOR₂ |
| 46 | |

| | | | |
|---|---|---|---|
| 47 | NMebAla | 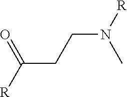 | CN(CCC([R])═O) [R] |

C₄H₇NOR₂

| | | | |
|---|---|---|---|
| 48 | aMeC | 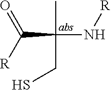 | C[c@](CS) (C([R])═O) N[R] C[C@](CS)(C═O)N |

C₄H-NOSR₂

| | | | |
|---|---|---|---|
| 49 | hC, hCys, homoC, or homoCys | 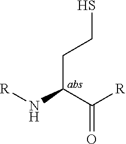 | O═C([C@H](CCS) N[R])[R] |

C₄H-NOSR₂

| | | | |
|---|---|---|---|
| 50 | iPrCO | 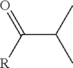 | CC(C)C([R])═O |

C₄H═OR

| | | | |
|---|---|---|---|
| 51 | dDab, dab, (R)-2,4- diaminobutanoic acid | 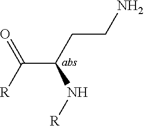 | NCC[C@H](C([R])═O)N[R] |

C₄H₈N₂OR₂

| | | | |
|---|---|---|---|
| 52 | homobAla | 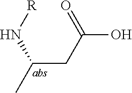 | C[C@@H](CC(O)═ O)N[R] |

C₄H₈NO₂R

| | | | |
|---|---|---|---|
| 53 | Bua, Butanoic acid | 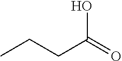 | CCCC(O)═O |

C₄H₈O₂

| | | | |
|---|---|---|---|
| 54 | Orn, ORN, Ornithine | 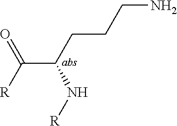 | NCCC[C@@H](C ([R])═O)N[R] |

| | |
|---|---|
| | C₅H₁₀N₂OR₂ |
| 55 | |

| | | | |
|---|---|---|---|
| 56 | Orn, L-ornithine | 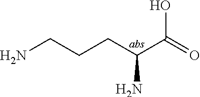 | NCCC[C@@H] (C(O)═O)N |

C₅H₁₂N₂O₂

| | | | |
|---|---|---|---|
| 57 | 4diFPro | 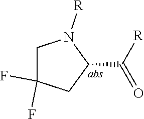 | O═C([C@H](CC(C 1)(F)F)N1[R])[R] O═C[C@H](C1)NC C1(F)F |

C₅H₅F₂NOR₂

| | | | |
|---|---|---|---|
| 58 | prG, prG, Fmoc-L- propargyl-Gly═OH, Pra | 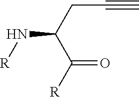 | C#CC[C@@H](C ([R])═O)N[R] |

C₅H₅NOR₂

| | | | |
|---|---|---|---|
| 59 | 4TriazolAla | 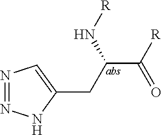 | O═C([C@H](Cc1cn n[nH]1)N[R])[R] |

C₅H₆N₄OR₂

| | | | |
|---|---|---|---|
| 60 | Tzl | 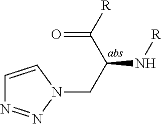 | O═C([C@H](Cn1n ncc1)N[R])[R] |

C₅H₆N₄OR₂

| | | | |
|---|---|---|---|
| 61 | PyE, PyE (S)-5-oxopyrrolidine- 2-carboxylic acid | 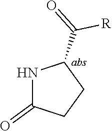 | O═C([C@H](CC1) NC1═O)[R] O═C[C@H](CC1)N C1═O |

C₅H₆NO₂R

| | | | |
|---|---|---|---|
| 62 | E(2) | 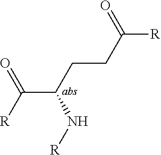 | O═C(CC[C@@H] (C([R])═O)N[R])[R] |

C₅H₆NO₂R₃

| | | | |
|---|---|---|---|
| 63 | Tetrazole | 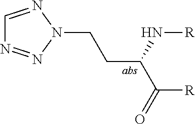 | O═C([C@H](CCn1 nncn1)N[R])[R] N[C@@H](CCn1n ncn1)C═O |

C₅H₇N₅OR₂

| | | | |
|---|---|---|---|
| 64 | 3OHPro | 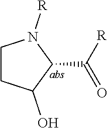 | OC(CC1)[C@@H] (C([R])═O)N1[R] |

C₅H₇NO₂R₂

| | | | |
|---|---|---|---|
| 65 | 4(R)HydroxyPro | 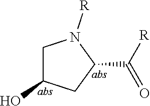 | O[C@H](C[C@H] 1C([R])═O)CN1[R] |

C₅H₇NO₂R₂

| | | | |
|---|---|---|---|
| 66 | Hyp | 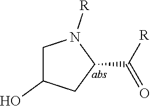 | OC(C[C@H]1C ([R])═O)CN1[R] |

C₅H₇NO₂R₂

| | | | |
|---|---|---|---|
| 67 | AllylGly | 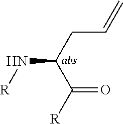 | C═CC[C@@H](C ([R])═O)N[R] |

C₅H₇NOR₂

| | | | |
|---|---|---|---|
| 68 | Dap(Ac) | 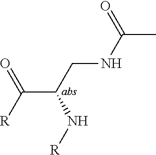 | CC(NC[C@@H](C ([R])═O)N[R])═O |

C₅H₈N₂O₂R₂

| | | | |
|---|---|---|---|
| 69 | N(NMe), NNMe, NMeAsn | 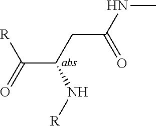 | CNC(C[C@@H] (C([R])═O)N[R])═O |

C₅H₈N₂O₂R₂

| | | | |
|---|---|---|---|
| 70 | aMeN. aMeAsn | 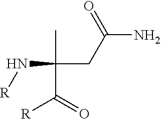 | C[C@](CC(N)═O) (C([R])═O)N[R] |

C₅H₈N₂O₂R₂

| | | | |
|---|---|---|---|
| 71 | 4(S)AminoPro | 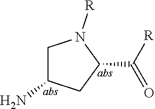 | N[C@@H](C[C@ H]1C([R])═O)CN1 [R] |

N₂OR₂

| | | | |
|---|---|---|---|
| 72 | CO(Morph) | 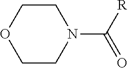 | O═C(N1CCOCC1) [R] |

C₅H₈NO₂R

| | | | |
|---|---|---|---|
| 73 | -COMorph, CO(Morph) | 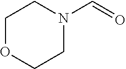 | O═CN1CCOCC1 |

| | |
|---|---|
| | C₅H₉NO₂ |
| 74 | |

| | | | |
|---|---|---|---|
| 75 | Nva | 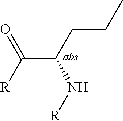 | CCC[C@@H](C ([R])═O)N[R] |

C₅H₉NOR₂

| | | | |
|---|---|---|---|
| 76 | dM, dMet, D- Methionine | 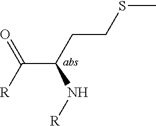 | CSCC[C@H](C ([R])═O)N[R] |

C₅H₉NOSR₂

| | | | |
|---|---|---|---|
| 77 | dPen, pen | 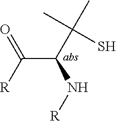 | CC(C)([C@H](C ([R])═O)N[R])S |

C₅H₉NOSR₂

| | | | |
|---|---|---|---|
| 78 | BuCO | 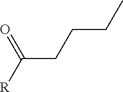 | CCCCC([R])═O |

C₅H₉OR

| | | | |
|---|---|---|---|
| 79 | iBuCO | 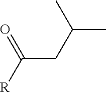 | CC(C)CC([R])═O CC[C@H](C)C ([R])═O |

C₅H₉OR

| | | | |
|---|---|---|---|
| 80 | tBuCO | 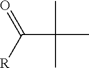 | CC(C)(C)C([R])═O |

C₅H₉OR

| | | | |
|---|---|---|---|
| 81 | N(N(Me)2), NNMe2 | 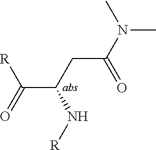 | CN(C)C(C[C@@H] (C([R])═O)N[R])═O |

C₆H₁₀N₂O₂R₂

| | | | |
|---|---|---|---|
| 82 | MorphCO, 2- morpholinoacetic acid | 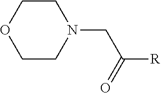 | O═C(CN1CCOCC1) [R] |

C₆H₁₀NO₂R

| | | | |
|---|---|---|---|
| 83 | CON(NMePip) | 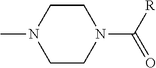 | CN(CC1)CCN1C ([R])═O |

C₆H₁₁N₂OR

| | | | |
|---|---|---|---|
| 84 | eK | 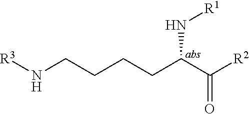 | O═C(O)[C@@H] (N[R])CCCCN[R] |

C₆H₁₁N₂OR₃

| | | | |
|---|---|---|---|
| 85 | Cit, Citrulline | 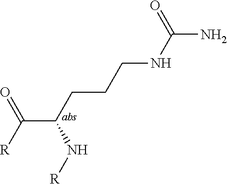 | NC(NCCC[C@@H] (C([R])═O)N[R])═O N[C@@H](CCCN C(N)═O)C(O)═O |

C₆H₁₁N₃O₂R₂

| | | | |
|---|---|---|---|
| 86 | D(NEtNH2) | 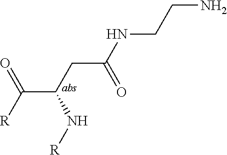 | NCCNC(C[C@@H] (C([R])═O)N[R])═O |

C₆H₁₁N₃02R₂

| | | | |
|---|---|---|---|
| 87 | Aad, 2-Aminoadipic acid | 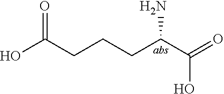 [SMILES: N[C@@H](CCCC(=O)O)C(=O)O] | N[C@@H](CCCC(O)═ O)C(O)═O |

C₆H₁₁NO₄

| | | | |
|---|---|---|---|
| 88 | N(Isobutyl)Gly | 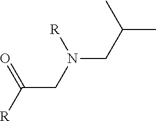 | CC(C)CN(CC ([R])═O)[R] |

C₆H₁₁NOR₂

| | | | |
|---|---|---|---|
| 89 | PentCO | 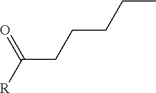 | CCCCCC([R])═O |

C₆H₁₁OR

| | | | |
|---|---|---|---|
| 90 | NMeQ, NMeGln, N- Methyl-Glutamine | 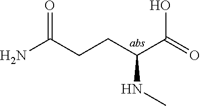 | CN[C@@H](CCC (N)═O)C(O)═O |

C₆H₁₂N₂O₃

| | | | |
|---|---|---|---|
| 91 | SP6 | 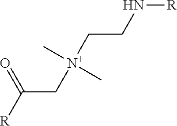 | C[N+](C)(CCN[R]) CC([R])═O C[N+](C)(CCN)CC═O |

C₆H₁₃N₂OR₂⁺

| | | | |
|---|---|---|---|
| 92 | 3IOxa4Ala | 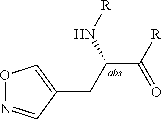 | O═C([C@H](Cc1co nc1)N[R])[R] |

C₆H₆N₂O₂R₂

| | | | |
|---|---|---|---|
| 93 | 3Oxa4Ala | 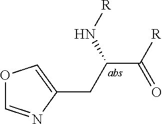 | O═C([C@H](Cc1co cn1)N[R])[R] |

C₆H₆N₂O₂R₂

| | | | |
|---|---|---|---|
| 94 | diFCpx | 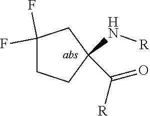 | O═C([C@](CC1)(C C1(F)F)N[R])[R] |

C₆H₇F₂NOR₂

| | | | |
|---|---|---|---|
| 95 | aMePra | 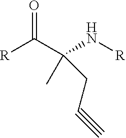 | C[C@](CC#C)(C ([R])═O)N[R] |

C₆H₇NOR₂

| | | | |
|---|---|---|---|
| 96 | CO(DiFPip) | 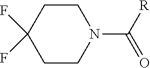 | O═C(N(CC1)CCC1 (F)F)[R] |

C₆H₈F₂NOR

| | | | |
|---|---|---|---|
| 97 | dab(COCH2(1*)) dab(COCH2)(1*) | 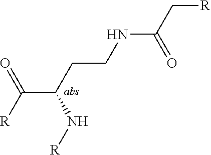 | O═C(C[R])NCC[C @@H](C([R])═O) N[R] |

C₆H₉N₂O₂R₃

| | | | |
|---|---|---|---|
| 98 | Tetrazole(NMe) | 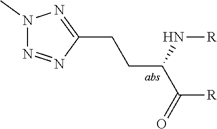 | Cn1nnc(CC[C@@ H](C([R])═O)N[R]) n1 |

| | | | |
|---|---|---|---|
| | | C₆H₉N₅OR₂ | |
| 99 | | | |
| 100 | dhE | 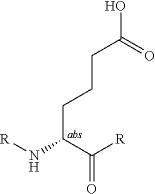 | OC(CCC[C@H](C ([R])═O)N[R])═O |
| | | C₆H₉NO₃R₂ | |
| 101 | Acpx | 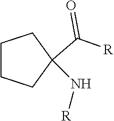 | O═C(C1(CCCC1)N [R])[R] NC1(CCCC1)C═O |
| | | C₆H₉NOR₂ | |
| 102 | aMeP, aMePro | 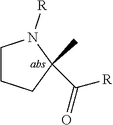 | C[c@](CCC1)(C ([R])═O)N1[R] |
| | | C₆H₉NOR₂ | |
| 103 | D(N2AmIm) | 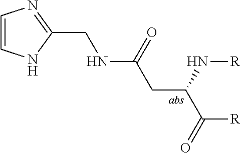 | O═C(C[C@@H](C ([R])═O)N[R])NCc 1ncc[nH]1 |
| | | C₈H₁₀N₄O₂R₂ | |
| 104 | KTfa, K(Tfa), L- Lys(Tfa) | 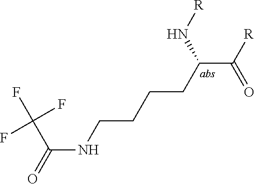 | O═C([C@H](CCC CNC(C(F)(F)F)═O) N[R])[R] |
| | | C₈H₁₁F₃N₂O₂R₂ | |
| 105 | E(OAll) | 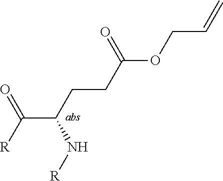 | C═CCOC(CC[C@ @H](C([R])═O)N [R])═O |
| | | C₈H₁₁NO₃R₂ | |
| 106 | D(NPyr) | 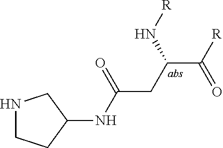 | O═C(C[C@@H](C ([R])═O)N[R])NC1 CNCC1 |
| | | C₈H₁₃N₃O₂R₂ | |
| 107 | Chg | 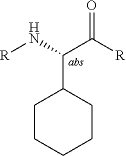 | O═C([C@H](C1CC CCC1)N[R])[R] |
| | | C₈H₁₃NOR₂ | |
| 108 | R5Me, aMeR5H | 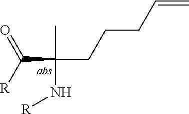 | C[C@@](CCCC═ C)(C([R])═O)N[R] |
| | | C₈H₁₃NOR₂ | |
| 109 | R6H, (R,E)-2-amino- 8-hydroxyoct-7-enoic acid | 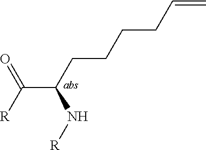 | C═CCCCC[C@H] (C([R])═O)N[R] C-CCCCC[C@H] (C═O)N |
| | | C₈H₁₃NOR₂ | |
| 110 | S5Me aMeS5H | 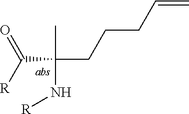 | C[C@](CCCC═C) (C([R])═O)N[R] |
| | | C₈H₁₃NOR₂ | |
| 111 | S6H | 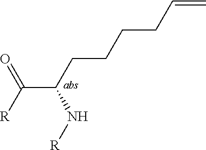 | C═CCCCC[C@@H] (C([R])═O)N[R] |
| | | C₈H₁₃NOR₂ | |
| 112 | KAc, K(Ac), K(COMe), K-Ac, N6-acetyl-L-Lysine | 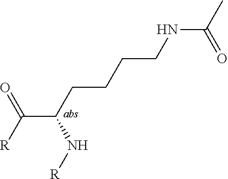 | CC(NCCCC[C@@ H](C([R])═O)N [R])═O |
| | | C₈H₁₄N₂O₂R₂ | |
| 113 | Pip(NMe2) | 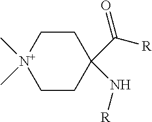 | C[N+](C)(CC1)CC C1(C([R])═O)N[R] |
| | | C₈H₁₅N₂OR₂⁺ | |
| 114 | K(Gly) | 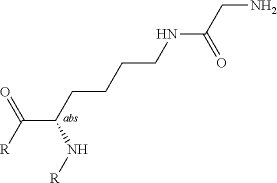 | NCC(NCCCC[C@ @H](C([R])═O)N [R])═O |
| | | C₈H₁₅N₃O₂R₂ | |
| 115 | 8Aoc, 8Aoc(2) | 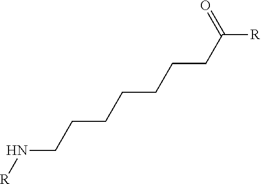 | O═C(CCCCCCCN [R])[R] |
| | | C₈H₁₅NOR₂ | |
| 116 | 2Benzyl | 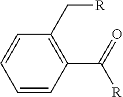 | O═C(c1c(C[R])cccc 1)[R] |
| | | C₈H₆OR₂ | |
| 117 | 6OH3Pya | 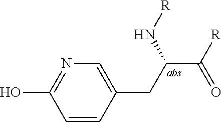 | Oc1ncc(C[C@@H] (C([R])═O)N[R])cc1 |
| | | C₈H₈N₂O₂R₂ | |
| 118 | 3Pya, 3Pal, 3-(2- pyridyl)-alanine | 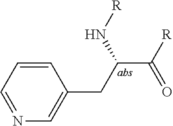 | O═C([C@H](Cc1cn ccc1)N[R])[R] |
| | | C₈H₈N₂OR₂ | |
| 119 | 4Pya, 4Pya, 4Pal, (S)- 2-amino-3-(pyridin-4- yl)propanoic acid 4PyridinAla | 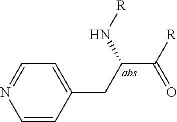 | O═C([C@H](Cc1cc ncc1)N[R])[R] |
| | | C₈H₃N₂OR₂ | |
| 120 | dPal, dpal, d3Pya, 3pya, 3- pyridylalanine, (R)-2- amino-3-(pyridin-3- yl)propanoic acid | 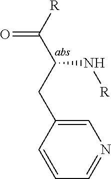 | O═C([C@@H](Cc1 cnccc1)N[R])[R] |
| | | C₈H₈N₂OR₂ | |
| 121 | 6MePyridazAla | 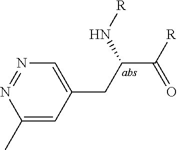 | Cc1cc(C[C@@H] (C([R])═O)N[R])cnn1 |
| | | C₈H₉N₃OR₂ | |
| 122 | 5MePyridinAla | 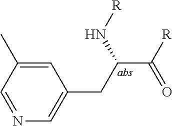 | Cc1cc(C[C@@H] (C([R])═O)N[R])cnc1 |
| | | C₉H₁₀N₂OR₂ | |
| 123 | J, Aph, 4- aminophenylalanine | 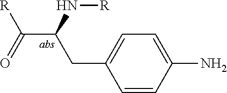 | Nc1ccc(C[C@@H] (C([R])═O)N[R])cc1 |
| | | C₉H₁₀N₂OR₂ | |
| 124 | NMe3Pya | 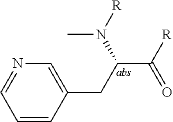 | CN([C@@H](Cc1c nccc1)C([R])═O)[R] CN[C@@H](Cc1c nccc1)C═O |
| | | C₉H₁₀N₂OR₂ | |
| 125 | SMSBCO | 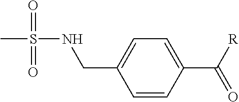 | CS(NCc(cc1)ccc1C ([R])═O)(═O)═O |
| | | C₉H₁₀NO₃SR | |
| 126 | Me3Pya | 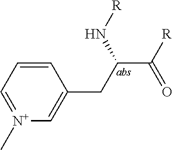 | C[n+]1cccc(C[C@ @H](C([R])═O)N [R])c1 |
| | | C₉H₁₁N₂OR₂⁺ | |
| 127 | D(Pip), (S)-2-amino- 4-oxo-4-(piperidin-1- yl)butanoic acid | 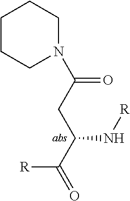 | O═C(C[C@@H](C ([R])═O)N[R])N1C CCCC1 |
| | | C₉H₁₄N₂O₂R₂ | |
| 128 | D(NPip) | 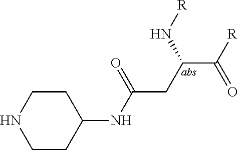 | O═C(C[C@@H](C ([R])═O)N[R])NC1 CCNCC1 |
| | | C₉H₁₅N₃O₂R₂ | |
| 129 | N(Cyclohexyl)Gly | 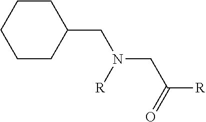 | O═C(CN(CC1CCC CC1)[R])[R] |
| | | C₉H₁₅NOR₂ | |
| 130 | R7H, (R,E)-2-amino- 9-hydroxynon-8- enoic acid | 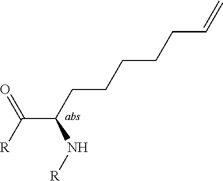 | C═CCCCCC[C@H] (C([R])═O)N[R] C═CCCCCC[C@H] (C═O)N |
| | | C₉H₁₅NOR₂ | |
| 131 | K(COEt) | 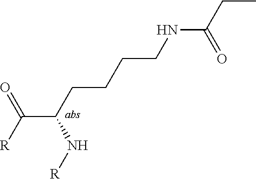 | CCC(NCCCC[C@ @H](C([R])═O)N [R])═O |
| | | C₉H₁₆N₂O₂R₂ | |
| 132 | K(NMeAc), KNMeAc | 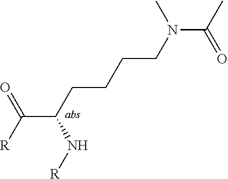 | CC(N(C)CCCC[C @@H](C([R])═O) N[R])═O |
| | | C₉H₁₆N₂O₂R₂ | |
| 133 | Q(NHtBu) | 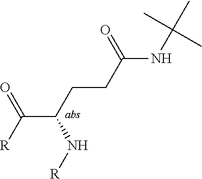 | CC(C)(C)NC(CC[C @@H](C([R])═O) N[R])═O |
| | | C₉H₁₆N₂O₂R₂ | |
| 134 | K(Me)3 | 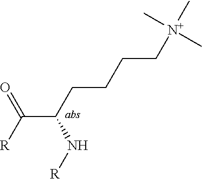 | C[N+](C)(C)CCCC [C@@H](C([R])═O)N[R] |
| | | C₉H₁₉N₂OR₂⁺ | |
| 135 | dK(Me)3, k(Me)3 | 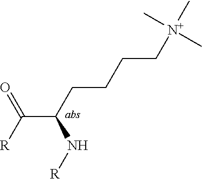 | C[N+](C)(C)CCCC [C@H](C([R])═O) N[R] |
| | | C₉H₁₉N₂OR₂⁺ | |
| 136 | 5cpaCO | 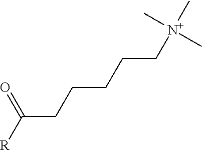 | C[N+](C)(C)CCCC CC([R])═O |
| | | C₉H₁₉NOR⁺ | |
| 137 | tetraFPhe | 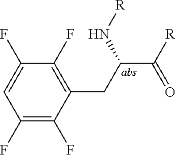 | O═C([C@H](Cc(c (F)c(cc1F)F)c1F)N[R]) [R] |
| | | C₉H₅F₄NOR₂ | |
| 138 | 5CF33Pya | 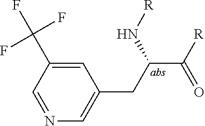 | O═C([C@H](Cc1cn cc(C(F)(F)F)c1)N[R]) [R] N[C@@H](Cc1cc (C(F)(F)F)cnc1)C═O |
| | | C₉H₇F₃N₂OR₂ | |
| 139 | 3,4diFPhe, 4diFPhe | 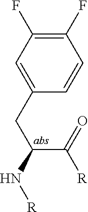 | O═C([C@H](Cc(cc 1)cc(F)c1F)N[R])[R] |
| | | C₉H₇F₂NOR₂ | |
| 140 | F(4N3) | 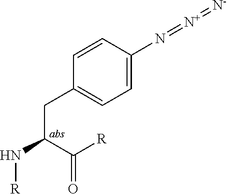 | [N-]═[N+]═Nc1ccc(C [C@@H](C([R])═ O)N[R])cc1 |
| | | C₉H₈N₄OR₂ | |
| 141 | 3FTyr | 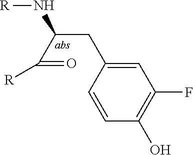 | Oc(ccc(C[C@@H] (C([R])═O)N[R])c1) c1F |
| | | C₉H₈FNO₂R₂ | |
| 142 | 2BrPhe, 2BrF | 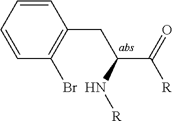 | O═C([C@H](Cc(cc cc1)c1Br)N[R])[R] |
| | | C₉H₈BrNOR₂ | |
| 143 | 2FPHE, 2FPhe | 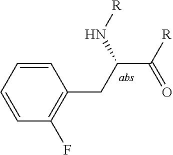 | O═C([C@H](Cc(cc cc1)c1F)N[R])[R] |
| | | C₉H₈FNOR₂ | |
| 144 | 3FPHE, 3FPhe | 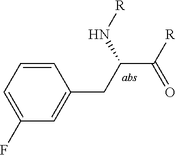 | O═C([C@H](Cc1cc (F)ccc1)N[R])[R] |
| | | C₉H₈FNOR₂ | |
| 145 | BHCO | 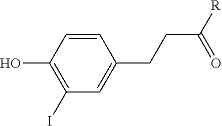 | Oc(ccc(CCC([R])═ O)c1)c1I |
| | | C₉H₈IO₂R | |
| 146 | 5 AmPyridinAla | 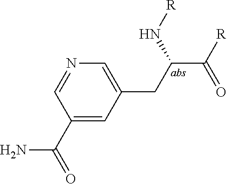 | NC(c1cc(C[C@@H] (C([R])═O)N[R]) cnc1)═O |
| | | C₉H₉N₃O₂R₂ | |
| 147 | mTYR, mY, mTyr | 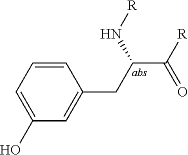 | Oc1cccc(C[C@@H] (C([R])═O)N[R])c1 |
| | | C₉H₉NO₂R₂ | |
| 148 | 6OHQuin | 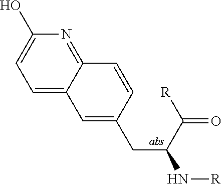 | Oc1ccc(cc(C[C@ @H](C([R])═O)N [Rl)cc2)c2n1 |
| | | C₁₂H₁₀N₂O₂R₂ | |
| 149 | 4AmF, 4AmPhe | 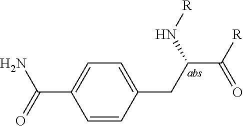 | NC(c1ccc(C[C@ @H](C([R])═O) N[R])cc1)═O N[C@@H](Cc(cc 1)ccc1C(N)═O)C═O |
| | | C₁₀H₁₀N₂O₂R₂ | |
| 150 | AEF(NMe(2)) | 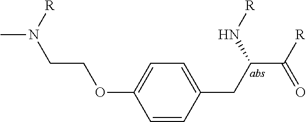 | CN(CCOc1ccc(C [C@@H](C([R])═ O)N[R])cc1)[R] |
| | | C₁₂H₁₅N₂O₂R₃ | |
| 151 | aMeY01 | 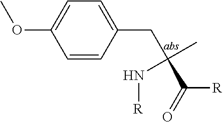 | C[C@](Cc(cc1)cc c1OC)(C([R])═O) N[R] |
| | | C₁₁H₁₃NO₂R₂ | |
| 152 | BiF | 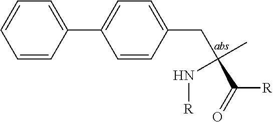 | C[C@](Cc(cc1)ccc1- c1ccccc1)(C([R])═ O)N[R] |
| | | C₁₆H₁₅NOR₂ | |
| 153 | hdKMe3, hk(Me)3 | 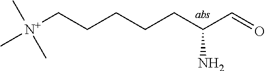 | C[N+](C)(C)CCC CC[C@H](C═O)N |
| | | C₁₀H₂₃N₂O+ | |
| 154 | Y(OTzl) | 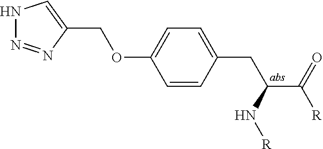 | O═C([C@H](Cc(c c1)ccc1OCc1c[nH] nn1)N[R])[R] |
| | | C₁₂H₁₂N₄O₂R₂ | |
| 155 | 3CONH2F | 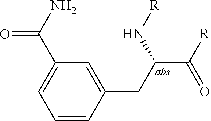 | NC(c1cccc(C[C@ @H](C([R])═O)N [R])c1)═O |
| | | C₁₀H₁₀N₂O₂R₂ | |
| 156 | 4AmDF, 4AmDPhe | 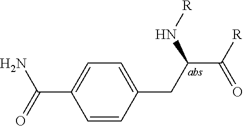 | NC(c1ccc(C[C@H] (C([R])═O)N[R]) cc1)═O |
| | | C₁₀H₁₀N₂O₂R₂ | |
| 157 | 4AmF, 4AmPhe | 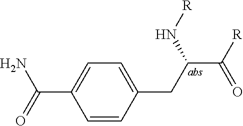 | NC(c1ccc(C[C@ @H](C([R])═O)N [R])cc1)═O N[C@@H](Cc(cc 1)ccc1C(N)═O)C═O |
| | | C₁₀H₁₀N₂O₂R₂ | |
| 158 | D(NPh) | 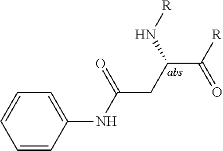 | O═C(C[C@@H] (C([R])═O)N[R])N c1ccccc1 |
| | | C₁₀H₁₀N₂O₂R₂ | |
| 159 | N(3AmBenzyl)Gly | 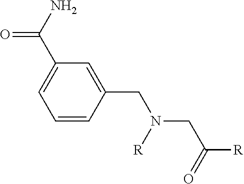 | NC(c1cccc(CN(C C([R])═O)[R]) c1)═O |
| | | C₁₀H₁₀N₂O₂R₂ | |
| 160 | N(4AmBenzyl)Gly | 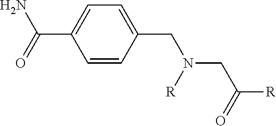 | NC(c1ccc(CN(CC ([R])═O)[R])cc1)═O |
| | | C₁₀H₁₀N₂O₂R₂ | |
| 161 | 2AmTyr | 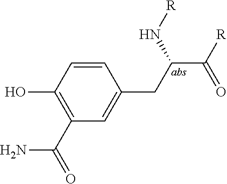 | NC(c(cc(C[C@@ H](C([R])═O)N[R]) cc1)c1O)═O |
| | | C₁₀H₁₀N₂O₃R₂ | |
| 162 | aMeFPhe | 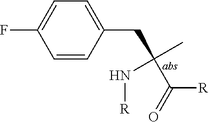 | C[C@](Cc(cc1)cc c1F)(C([R])═O)N [R] |
| | | C₁₀H₁₀FNOR₂ | |
| 163 | D(NmAn) | 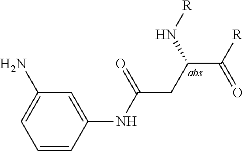 | Nc1cccc(NC(C[C @@H](C([R])═O) N[R])═O)c1 |
| | | C₁₀H₁₁N₃O₂R₂ | |
| 164 | D(NoAn) | 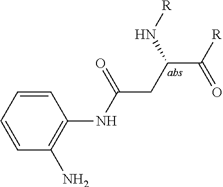 | Nc(cccc1)c1NC(C [C@@H](C([R])═ O)N[R])═O |
| | | C₁₀H₁₁N₃O₂R₂ | |
| 165 | D(NpAn) | 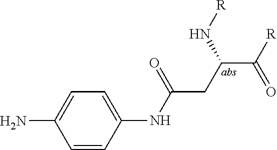 | Nc(cc1)ccc1NC(C [C@@H](C([R])═ O)N[R])═O |
| | | C₁₀H₁₁N₃O₂R₂ | |
| 166 | 4MeOF | 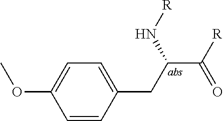 | COc1ccc(C[C@@ H](C([R])═O)N [R])cc1 COc1ccc(C[C@@ H](C═O)N)cc1 |
| | | C₁₀H₁₁NO₂R₂ | |
| 167 | NMeDTyr, NMeDY, NMedTyr, NMedY, N-Methyl-D-tyrosine, dNMeTyr dNMeY | 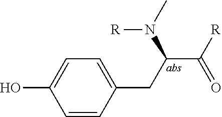 | CN([C@H](Cc(cc 1)ccc1O)C([R])═O) [R] |
| | | C₁₀H₁₁NO₂R₂ | |
| 168 | aMe3OHPhe | 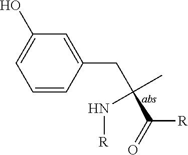 | C[c@](Cc1cc(O)c cc1)(C([R])═O)N[R] |
| | | C₁₀H₁₁NO₂R₂ | |
| 169 | aMeY, aMe Tyr | 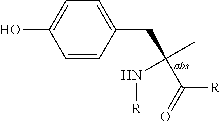 | C[C@](Cc(cc1)cc c1O)(C([R])═O)N[R] |
| | | C₁₀H₁₁NO₂R₂ | |
| 170 | bMeDTyr(2R₃S) bMeDTyr(2R,3S) | 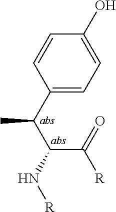 | C[C@H]([C@H] (C([R])═O)N[R])c (cc1)ccc1O |
| | | C₁₀H₁₁NO₂R₂ | |
| 171 | 4MeF | 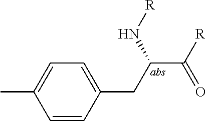 | Cc1ccc(C[C@@H] (C([R])═O)N[R]) cc1 |
| | | C₁₀H₁₁NOR₂ | |
| 172 | aMeF, aMeF alpha-methyl phenylalanine | 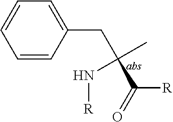 | C[C@](Cc1ccccc1) (C([R])═O)N[R] C[C@](Cc1ccccc1) (C═O)N |
| | | C₁₀H₁₁NOR₂ | |
| 173 | bMePhe | 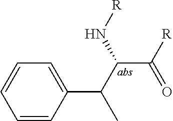 | CC([C@@H](C ([R])═O)N[R])c1ccc cc1 |
| | | C₁₀H₁₁NOR₂ | |
| 174 | bMePhe(2S3S) bMePhe(2S,3S) | 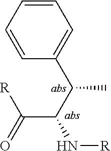 | C[C@H]([C@@H (C([R])═O)N[R]) c1ccccc1 |
| | | C₁₀H₁₁NOR₂ | |
| 175 | hF, hPhe, homoF, homoPhe | 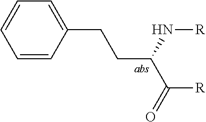 | O═C([C@H](CCc 1ccccc1)N[R])[R] |
| | | C₁₀H₁₁NOR₂ | |
| 176 | F₄CONH2, 4- carbamoyl-L- phenylalanine | 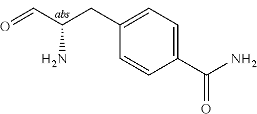 [SMILES: NC(=O)c1ccc(C[C@H](N)C=O)cc1] | N[C@@H](Cc(cc 1)ccc1C(N)═ O)C═O |
| | | C₁₀H₁₂N₂O₂ | |
| 177 | Maf | 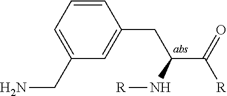 | NCc1cccc(C[C@ @H](C([R])═O)N [R])c1 |
| | | C₁₀H₁₂N₂OR₂ | |
| 178 | Paf | 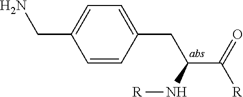 | NCc1ccc(C[C@@ H](C([R])═O)N[R]) cc1 NCc1ccc(C[C@@ H](C═O)N)cc1 |
| | | C₁₀H₁₂N₂OR₂ | |
| 179 | dMaf, maf | 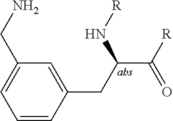 | NCc1cccc(C[C@ H](C([R])═O)N[R])c1 |
| | | C₁₀H₁₂N₂OR₂ | |
| 180 | dPaf | 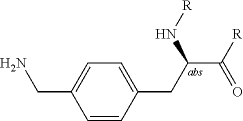 | NCc1ccc(C[C@H] (C([R])═O)N[R])c c1 |
| | | C₁₀H₁₂N₂OR₂ | |
| 181 | oAMPhe | 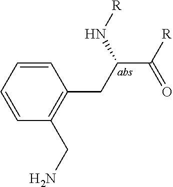 | NCc1c(C[C@@H] (C([R])═O)N[R])c ccc1 |
| | | C₁₀H₁₂N₂OR₂ | |
| 182 | F(4G) | 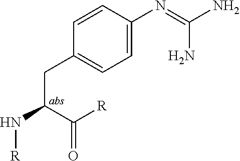 | NC(N)═Nc1ccc(C [C@@H](C([R])═ O)N[R])cc1 |
| | | C₁₀H₁₂N₄OR₂ | |
| 183 | NMeDTyr | 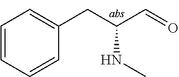 | CN[C@H](Cc1ccc cc1)C═O |
| | | C₁₀H₁₃NO | |
| 184 | dNMeTyr dNMeY, D-N-methyl tyrosine N-Methyl-D-tyrosine | 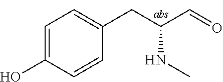 | CN[C@H](Cc(cc1) ccc1O)C═O |
| | | C₁₀H₁₃NO₂ | |
| 185 | biotin | 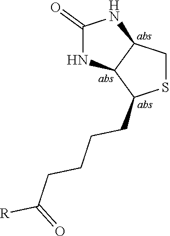 | O═C(CCCC[C@ @H\|([C@H]1N2) SC[C@@H]1NC2═ O)[R] |
| | | C₁₀H₁₅N₂O₂SR | |
| 186 | K(CO2allyl) | 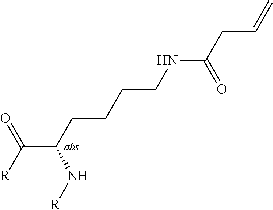 | C═CCC(NCCCC[C @@H](C([R])═ O)N[R])═O C═CCOC(NCCCC [C@@H](C([R])═ O)N[R])═O |
| | | C₁₀H₁₆N₂O₂R₂ | |
| 187 | K(COcPr) | 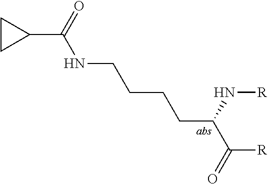 | O═C([C@H](CCC CNC(C1CC1)═O) N[R])[R] |
| | | C₁₀H₁₆N₂O₂R₂ | |
| 188 | DAGSuc | 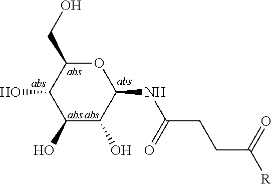 | OC[C@H]([C@H] ([C@@H]([C@H] 1O)O)O)O[C@H] 1NC(CCC([R])═ O)═O |
| | | C₁₀H₁₆NO₇R | |
| 189 | K(COPr) | 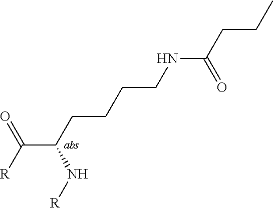 | CCCC(NCCCC[C @@H](C([R])═O) N[R])═O |
| | | C₁₀H₁₈N₂O₂R₂ | |
| 190 | K(COiPr) | 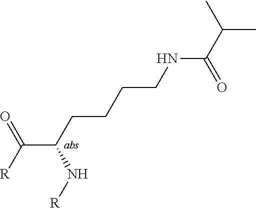 | CC(C)C(NCCCC [C@@H](C([R])═ O)N[R])═O |
| | | C₁₀H₁₈N₂O₂R₂ | |
| 191 | Tzl(Ch) | 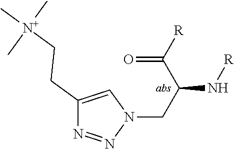 | C[N+](C)(C)CCc1 cn(C[C@@H](C ([R])═O)N[R])nn1 |
| | | C₁₀H₁₈N₅OR₂⁺ | |
| 192 | hK(Me)3, hKMe3 | 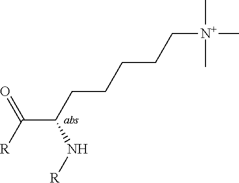 | C[N+](C)(C)CCC CC[C@@H](C ([R])═O)N[R])nn1 |
| | | C₁₀H₂₁N₂OR₂⁺ | |
| 193 | hdK(Me)3, hk(Me)3, hdKMe3 | 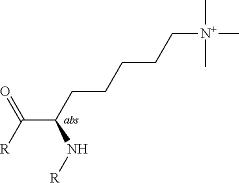 | C[N+](C)(C)CCC CC[C@H](C ([R])═O)N[R] |
| | | C₁₀H₂₁N₂OR₂⁺ | |
| 194 | Dap(pF(6)) | 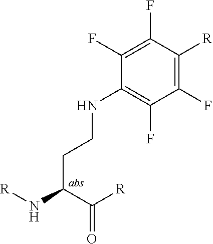 | O═C([C@H](CCN c(c(F)c(c([R])c1F) F)c1F)N[R])[R] |
| | | C₁₀H₇F₄N₂OR₃ | |
| 195 | 4OCF3DPhe | 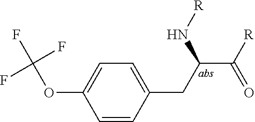 | O═C([C@@H](Cc (cc1)ccc1OC(F)(F) F)N[R])[R] |
| | | C₁₀H₈F₃NO₂R₂ | |
| 196 | CF3F | 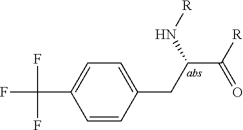 | O═C([C@H](Cc1c cc(C(F)(F)F)cc1)N [R])[R] |
| | | C₁₀H₈F₃NOR₂ | |
| 197 | 7AzaW | 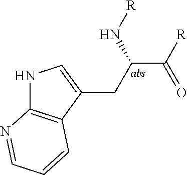 | O═C([C@H](Cc1c [nH]c2c1cccn2)N [R])[R] |
| | | C₁₀H₉N₃OR₂ | |
| 198 | Y(CHF2) | 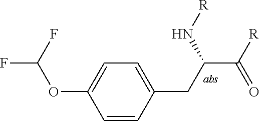 | O═C([C@H](Cc(c c1)ccc1OC(F)F)N [R])[R] |
| | | C₁₀H₉F₂NO₂R₂ | |
| 199 | CXF | 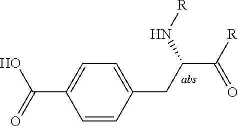 | OC(c1ccc(C[C@ @H](C([R])═O)N [R])cc1)═O |
| | | C₁₀H₉NO₃R₂ | |
| 200 | CHF₂Phe | 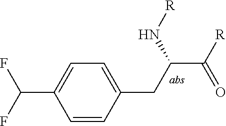 | O═C([C@H](Cc1c cc(C(F)F)cc1)N[R]) [R] |
| | | C₁₀H₉F₂NOR₂ | |
| 201 | TetraFAEF | 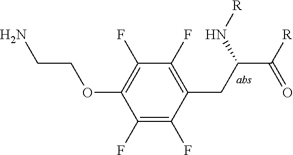 | NCCOc(c(F)c(c(C [C@@H](C([R])═ O)N[R])c1F)F)c1F |
| | | C₁₁H₁₀F₄N₂O₂R₂ | |
| 202 | 5OHW | 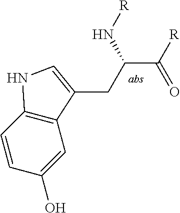 | Oc(cc1)cc2c1[nH] cc2C[C@@H] (C ([R])═O)N[R] |
| | | C₁₁H₁₀N₂O₂R₂ | |
| 203 | 4AcDPhe | 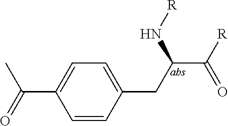 | CC(c1ccc(C[C@H] (C([R])═O)N[R]) cc1)═O |
| | | C₁₁H₁₁NO₂R₂ | |
| 204 | D(NBzl) | 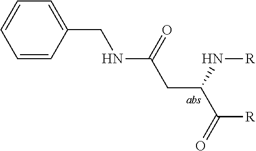 | O═C(C[C@@H] (C([R])═O)N[R])N Cc1ccccc1 |
| | | C₁₁H₁₂N₂O₂R₂ | |
| 205 | aMe2AmTyr | 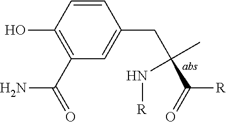 | C[C@](Cc(cc1)cc (C(N)═O)c1O)(C ([R])═O)N[R] |
| | | C₁₁H₁₂N₂O₃R₂ | |
| 206 | psiW | 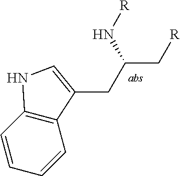 | [R]C[C@H](Cc1c [nH]c2c1cccc2)N [R] |
| | | C₁₁H₁₂N₂R₂ | |
| 207 | aMeY01 | 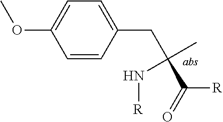 | C[C@](Cc(cc1)cc c1OC)(C([R])═O) N[R] |
| | | C₁₁H₁₃NO₂R₂ | |
| 208 | 3OMeY01 | 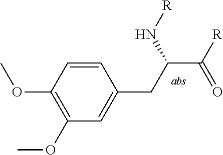 | COc(ccc(C[C@@ H](C([R])═O)N[R]) c1)c1OC |
| | | C₁₁H₁₃NO₃R₂ | |
| 209 | | | |
| 210 | dAEF | 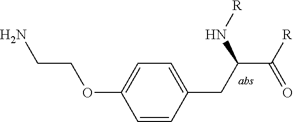 | NCCOc1ccc(C[C @H](C([R])═O)N [R])cc1 |
| | | C₁₁H₁₄N₂O₂R₂ | |
| 211 | K(COBu) | 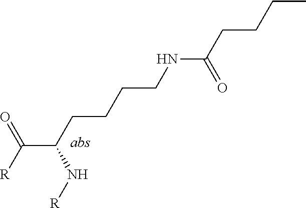 | CCCCC(NCCCC [C@@H](C([R])═ O)N[R])═O |
| | | C₁₁H₂₀N₂O₂R₂ | |
| 212 | K(COiBu) | 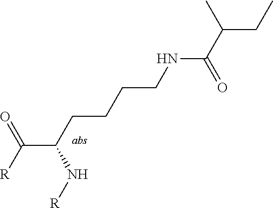 | CCC(C)C(NCCCC [C@@H](C([R])═ O)N[R])═O CC(C)CC(NCCCC [C@@H](C([R])═ O)N[R])═O |
| | | C₁₁H₂₀N₂O₂R₂ | |
| 213 | K(COtBu) | 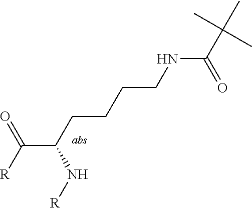 | CC(C)(C)C(NCCC C[C@@H](C ([R])═O)N[R])═O |
| | | C₁₁H₂₀N₂O₂R₂ | |
| 214 | succiniccarn | 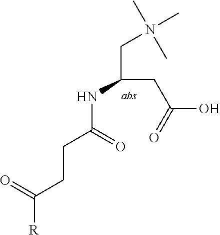 | C[N+](C)(C)C[C @@H](CC(O)═O) NC(CCC([R])═ O)═O |
| | | C₁₁H₂₀N₂O₄R⁺ | |
| 215 | Aun | 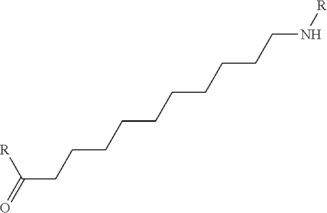 | O═C(CCCCCCCC CCN[R])[R] |
| | | C₁₁H₂₁NOR₂ | |
| 216 | 5BrW, 5BrTrp | 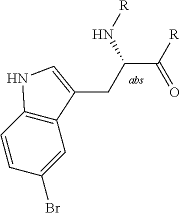 | O═C([C@H](Cc1c [nH]c(cc2)c1cc2Br) N[R])[R] |
| | | C₁₁H₉BrN₂OR₂ | |
| 217 | 7BrTrp, 7BrW | 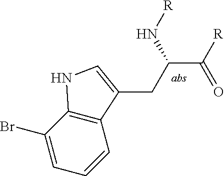 | O═C([C@H](Cc1c [nH]c2c1cccc2Br) N[R])[R] |
| | | C₁₁H₉BrN₂OR₂ | |
| 218 | 7ClW, 7ClTrp | 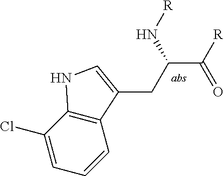 | O═C([C@H](Cc1c [nH]c2c1cccc2Cl) N[R])[R] |
| | | C₁₁H₉ClN₂OR₂ | |
| 219 | 5FW, 5FTrp | 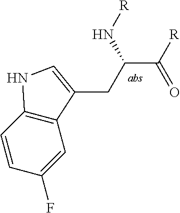 | O═C([C@H](Cc1c [nH]c(cc2)c1cc2F) N[R])[R] |
| | | C₁₁H₉FN₂OR₂ | |
| 220 | 7FW, 7FTrp | 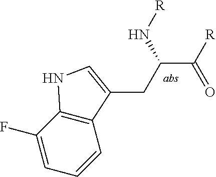 | O═C([C@H](Cc1c [nH]c2c1cccc2F)N [R])[R] |
| | | C₁₁H₉FN₂OR₂ | |
| 221 | BT, L-3- Benzothienylalanine | 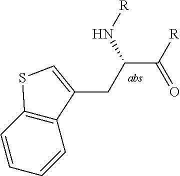 | O═C([C@H](Cc1c sc2c1cccc2)N[R]) [R] |
| | | C₁₁H₉NOSR₂ | |
| 222 | 2Quin 6OHQui | 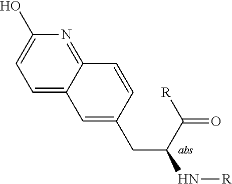 | Oc1ccc(cc(C[C@ @H](C([R])═O)N [R])cc2)c2n1 |
| | | C₁₂H₁₀N₂O₂R₂ | |
| 223 | 7CF2H | 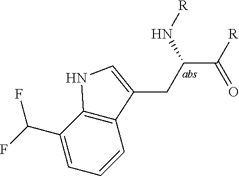 | O═C([C@H](Cc1c [nH]c2c1cccc2C (F)F)N[R])[R] |
| | | C₁₂H₁₀F₂N₂OR₂ | |
| 224 | 3QuinolAla | 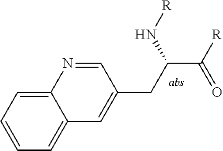 | O-C([C@H](Cc1c c2ccccc2nc1)N[R])[R] |
| | | C₁₂H₁₀N₂OR₂ | |
| 225 | 2MeTrp, 2MeW | 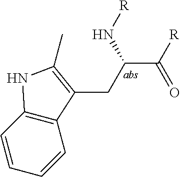 | Cc1c(C[C@@H] (C([R])═O)N[R])c (cccc2)c2[nH]1 |
| | | C₁₂H₁₂N₂OR₂ | |
| 226 | 5MeW, 5MeTrp | 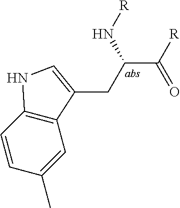 | Cc(cc1)cc2c1[nH] cc2C[C@@H](C ([R])═O)N[R] |
| | | C₁₂H₁₂N₂OR₂ | |
| 227 | 7MeW, 7(MeW), 7MeTrp | 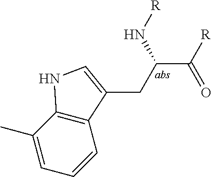 | Cc1cccc2c1[nH]cc 2C[C@@H](C ([R])═O)N[R] Cc1cccc2c1[nH]cc 2C[C@@H](C═O)N |
| | | C₁₂H₁₂N₂OR₂ | |
| 228 | aMeW | 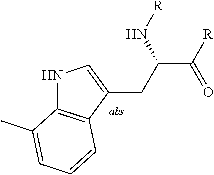 | C[C@](Cc1c[nH]c 2c1cccc2)(C([R])═ O)N[R] |
| | | C₁₂H₁₂N₂OR₂ | |
| 229 | dW7Me, 7Mew, 7MedW | 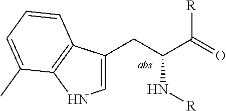 | Cc1cccc2c1[nH]cc 2C[C@H](C([R])═ O)N[R] |
| | | C₁₂H₁₂N₂OR₂ | |
| 230 | Y(OTzl) | 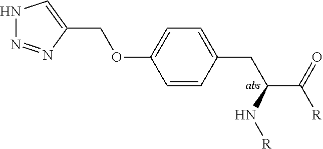 | O═C([C@H](Cc(c c1)ccc1OCc1c[nH] nn1)N[R])[R] |
| | | C₁₂H₁₂N₄O₂R₂ | |
| 231 | 4AllylY | 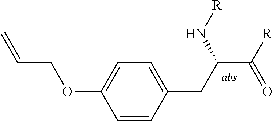 | C═CCOc1ccc(C[C @@H](C([R])═O) N[R])cc1 |
| | | C₁₂H₁₃NO₂R₂ | |
| 232 | 4AllylF | 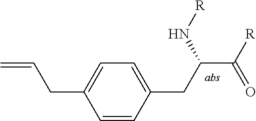 | C═CCc1ccc(C[C N[R])cc1 |
| | | C₁₂H₁₃NOR₂ | |
| 233 | meW, NMeW, NMeTrp, N-Methyl- Tryptophan | 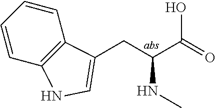 [SMILES: CN[C@@H](Cc1c[nH]c2ccccc12)C(=O)O] | CN[C@@H](Cc1c [nH]c2c1cccc2)C (O)═O |
| | | C₁₂H₁₄N₂O₂ | |
| 234 | AAMPhe | 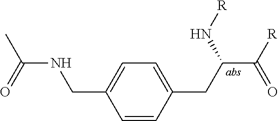 | CC(NCc1ccc(C[C @@H\|(C([R])═O) N[R])cc1)═O CC(NCc1ccc(C[C @@H](C═O)N) cc1)═O |
| | | C₁₂H₁₄N₂O₂R₂ | |
| 235 | hC(pXyl) | 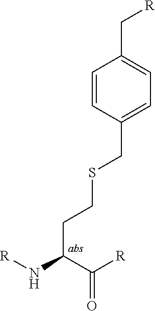 | O═C([C@H](CCS Cc1ccc(C[R])cc1) N[R])[R] |
| | | C₁₂H₁₄NOSR₃ | |
| 236 | AEF(NMe(2)) | 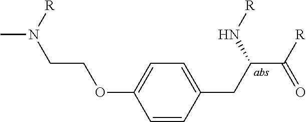 | CN(CCOc1ccc(C[ C@@H](C([R])═ O)N[R])cc1)[R] |
| | | C₁₂H₁₅N₂O₂R₃ | |
| 237 | DY02 | 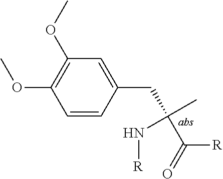 | C[C@@](Cc(cc1) cc(OC)c1OC)(C ([R])═O)N[R] |
| | | C₁₂H₁₅NO₃R₂ | |
| 238 | Y02 | 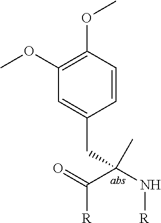 | C[C@](Cc(cc1)cc (OC)c1OC)(C([R])═O) N[R] |
| | | C₁₂H₁₅NO₃R₂ | |
| 239 | AEF(NMe) | 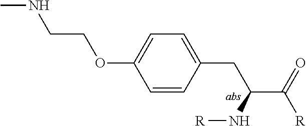 | CNCCOc1ccc(C[C @@H](C([R])═O) N[R])cc1 |
| | | C₁₂H₁₆N₂O₂R₂ | |
| 240 | NMeAEF | 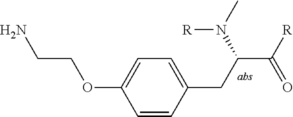 | CN([C@@H](Cc (cc1)ccc1OCCN)C ([R])═O)[R] CN[C@@H](Cc(c c1)ccc1OCCN)C═O |
| | | C₁₂H₁₆N₂O₂R₂ | |
| 241 | aMeAEF | 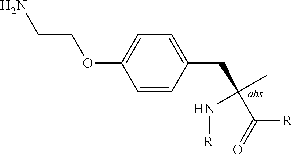 | C[C@](Cc(cc1)cc c1OCCN)(C([R])═ O)N[R] CC(C)(C)OC(NC COc1ccc(C[C@@ (C)(C([R])═O)N [R])cc1)═O |
| | | C₁₂H₁₆N₂O₂R₂ | |
| 242 | bMeAEF | 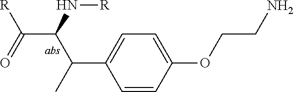 | CC([C@@H](C ([R])═O)N[R])c(cc1) ccc1OCCN |
| | | C₁₂H₁₆N₂O₂R₂ | |
| 243 | bMeAEF(2S,3R*), bMeAEF(2S3R*) (*pure but configuration unknown) | 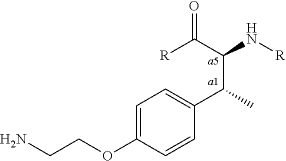 | C[c@@H]([C@ @H](C([R])═O)N [R])c(cc1)ccc1OCCN |
| | | C₁₂H₁₆N₂O₂R₂ | |
| 244 | bMeAEF(2S3S*), bMeAEF(2S,3S*) (*pure but configuration unknown) | 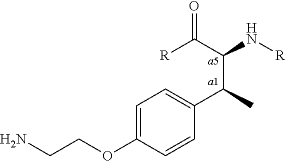 | C[C@H]([C@@H] (C([R])═O)N[R]) c(cc1)ccc1OCCN |
| | | C₁₂H₁₆N₂O₂R₂ | |
| 245 | K(Morph) | 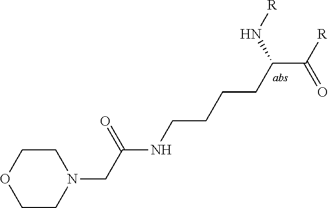 | O═C(CN1CCOCC 1)NCCCC[C@@ H](C([R])═O)N[R] |
| | | C₁₂H₂₁N₃O₃R₂ | |
| 246 | K(COPent) | 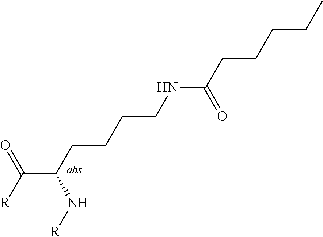 | CCCCCC(NCCCC [C@@H](C([R])═ O)N[R])═O |
| | | C₁₂H₂₂N₂O₂R₂ | |
| 247 | aMeK(Boc) | 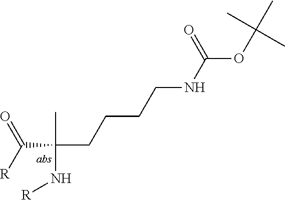 | CC(C)(C)OC(NC CCC[C@@](C)(C ([R])═O)N[R])=O |
| | | C₁₂H₂₂N₂O₃R₂ | |
| 248 | E(C) | 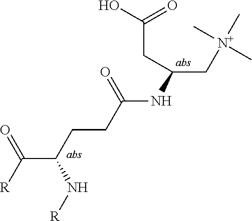 | C[N+](C)(C)C[C @H](CC(O)═O)N C(CC[C@@H](C ([R])═O)N[R])=O |
| | | C₁₂H₂₂N₃O₄R₂⁺ | |
| 249 | E(c) (R)-2-((R)-4-amino- 4- carboxybutanamido)- 3-carboxy-N,N,N- trimethylpropan-1- aminium, E(c) | 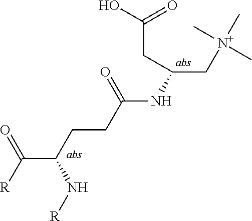 | C[N+](C)(C)C[C @@H](CC(O)═O) NC(CC[C@@H] (C([R])═O)N[R])═O |
| | | C₁₂H₂₂N₃O₄R₂⁺ | |
| 250 | e(C), dE(C) | 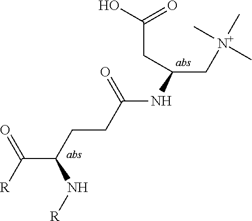 | C[N+](C)(C)C[C @H](CC(O)═O)N C(CC[C@H](C([R])═ O)N[R])═O |
| | | C₁₂H₂₂N₃O₄R₂⁺ | |
| 251 | e(c), dE(c) | 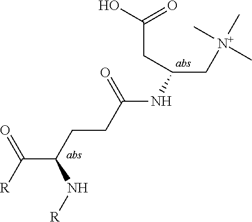 | C[N+](C)(C)C[C @@H](CC(O)═O) NC(CC[C@H](C ([R])═O)N[R])═O |
| | | C₁₂H₂₂N₃O₄R₂⁺ | |
| 252 | dK(SP6), k(SP6) | 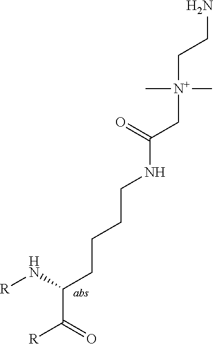 | C[N+](C)(CCN)C C(NCCCC[C@H] (C([R])═O)N[R])═ O |
| | | C₁₂H₂₅N₄O₂R₂⁺ | |
| 253 | 7CF3W, (S)-2-amino- 3-(7- (trifluoromethyl)-1H- indol-3-yl)propanoic acid | 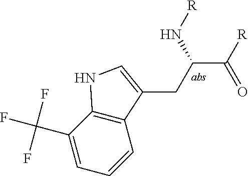 | O═C([C@H](Cc1c [nH]c2c1cccc2C(F) (F)F)N[R])[R] N[C@@H](Cc1c [nH]c2c(C(F)(F)F)c ccc12)C═O N[C@@H](Cc1c [nH]c2c(C(F)(F)F)c ccc12)C═O |
| | | C₁₂H₉F₃N₂OR₂ | |
| 254 | 5Br2Nal | 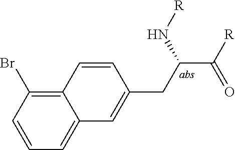 | O═C([C@H](Cc1c c2cccc(Br)c2cc1) N[R])[R] |
| | | C₁₃H₁₀BrNOR₂ | |
| 255 | 6Br2Nal | 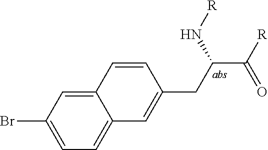 | O═C([C@H](Cc(c cc1c2)cc1ccc2Br) N[R])[R] |
| | | C₁₃H₁₀BrNOR₂ | |
| 256 | 7Br2Nal | 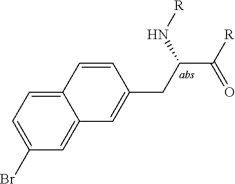 | O═C([C@H](Cc1c c2cc(Br)ccc2cc1) N[R])[R] |
| | | C₁₃H₁₀BrNOR₂ | |
| 257 | 6F2Nal | 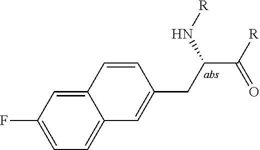 | O═C([C@H](Cc(c cc1c2)cc1ccc2F)N [R])[R] N[C@@H](Cc1cc c(cc(cc2)F)c2c1) C═O |
| | | C₁₃H₁₀FNOR₂ | |
| 258 | 7OH2Nal | 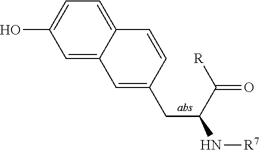 | Oc1ccc(ccc(C[C@ @H](C([R])═O)N [R1])c2)c2c1 |
| | | C₁₃H₁₁NO₂R₂ | |
| 259 | 1Nal, Nal, | 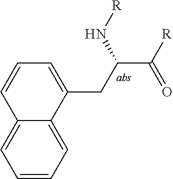 | O═C([C@H](Cc1c ccc2ccccc12)N[R]) [R] |
| | | C₁₃H₁₁NOR₂ | |
| 260 | 2Nal | 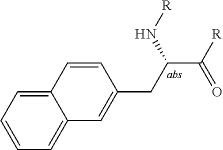 | O═C([C@H](Cc1c c2ccccc2cc1)N[R]) [R] |
| | | C₁₃H₁₁NOR₂ | |
| 261 | dNal, d2Nal | 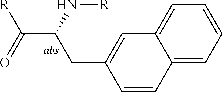 | O═C([C@@H](Cc 1cc2ccccc2cc1)N [R])[R] |
| | | C₁₃H₁₁NOR₂ | |
| 262 | 6MeQui | 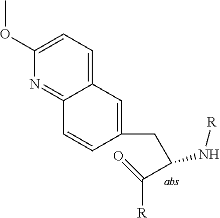 | COc1ccc(cc(C[C @@H](C([R])═O) N[R])cc2)c2n1 |
| | | C₁₃H₁₂N₂O₂R₂ | |
| 263 | D(N5In) | 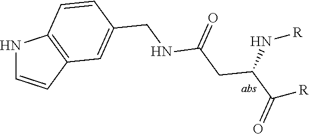 | O═C(C[C@@H] (C([R])═O)N[R])N Cc(cc1)cc2c1[nH] cc2 |
| | | C₁₃H₁₃N₃O₂R₂ | |
| 264 | psi2Nal | 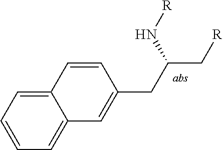 | [R]C[C@H](Cc1c c2ccccc2cc1)N[R] |
| | | C₁₃H₁₃NR₂ | |
| 265 | 7EtW | 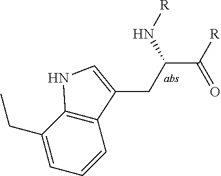 | CCc1cccc2c1[nH] cc2C[C@@H](C ([R])═O)N[R] |
| | | C₁₃H₁₄N₂OR₂ | |
| 266 | F(4TzIMME) | 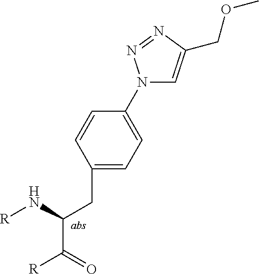 | COCc1cn(- c2ccc(C[C@@H] (C([R])═O)N[R])cc 2)nn1 |
| | | C₁₃H₁₄N₄O₂R₂ | |
| 267 | AcAEF | 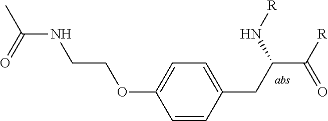 | CC(NCCOc1ccc(C [C@@H](C([R])═ O)N[R])cc1)═O |
| | | C₁₃H₁₆N₂O₃R₂ | |
| 268 | tButY, Y(tBu) | 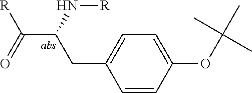 | CC(C)(C)Oc1ccc (C[C@H](C([R])═ O)N[R])cc1 |
| | | C₁₃H₁₇NO₂R₂ | |
| 269 | AEF(Me)2 | 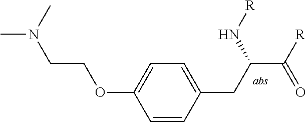 | CN(C)CCOc1ccc (C[C@@H](C([R])═ O)N[R])cc1 |
| | | C₁₃H₁₈N₂O₂R₂ | |
| 270 | Z, Amp | 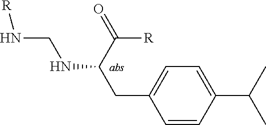 | CC(C)c1ccc(C[C @@H](C([R])═O) NCN[R])cc1 |
| | | C₁₃H₁₈N₂OR₂ | |
| 271 | 5amido2Nal | 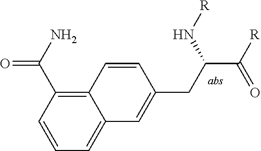 | NC(c1c(ccc(C[C@ @H](C([R])═O)N [R])c2)c2ccc1)═O |
| | | C₁₄H₁₂N₂O₂R₂ | |
| 272 | 6amido2Nal | 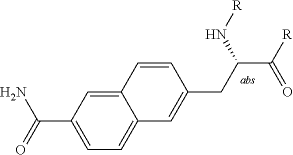 | NC(c1ccc(cc(C[C @@H](C([R])═O) N[R])cc2)c2c1)═O |
| | | C₁₄H₁₂N₂O₂R₂ | |
| 273 | 50Me2Nal | 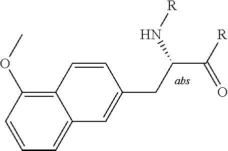 | COc1c(ccc(C[C@ @H](C([R])═O)N [R])c2)c2ccc1 |
| | | C₁₄H₁₃NO₂R₂ | |
| 274 | 6OMe2Nal | 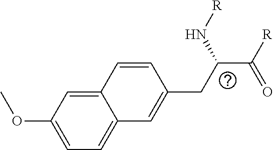 | COc1ccc(cc(C[C @@H](C([R])═O) N[R])cc2)c2c1 |
| | | C₁₄H₁₃NO₂R₂ | |
| 275 | 5Me2Nal | 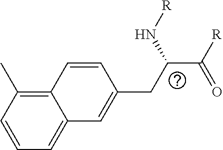 | Cc1c(ccc(C[C@@ H](C([R])═O)N[R]) c2)c2ccc1 |
| | | C₁₄H₁₃NOR₂ | |
| 276 | NMe2NAL | 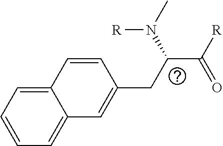 | CN([C@@H](Cc1 cc2ccccc2cc1)C ([R])═O)[R] CN[C@@H](Cc1c c2ccccc2cc1)C═O |
| | | C₁₄H₁₃NOR₂ | |
| 277 | aMe2Nal | 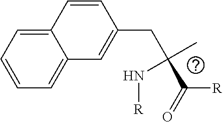 | C[C@](Cc1cc2ccc cc2cc1)(C([R])═O) N[R] |
| | | C₁₄H₁₃NOR₂ | |
| 278 | bMe2Nal(2S,3R), bMe2Nal(2S3R) | 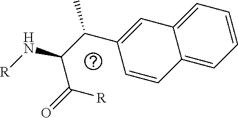 | C[C@@H]([C@ @H](C([R])═O)N [R])c1cc2ccccc2cc 1 |
| | | C₁₄H₁₃NOR₂ | |
| 279 | bMe2Nal(2S3S), bMe2Nal(2S3R) | 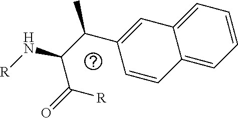 | C[C@H]([C@@H] (C([R])═O)N[R]) c1cc2ccccc2cc1 |
| | | C₁₄H₁₃NOR₂ | |
| 280 | AEF(EtCO) | 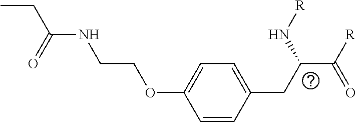 | CCC(NCCOc1ccc (C[C@@H](C([R])═O) N[R])cc1)═O |
| | | C₁₄H₁₈N₂O₃R₂ | |
| 281 | NMeY(tBu) | 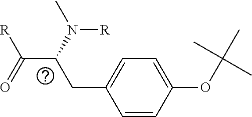 | CC(C)(C)Oc1ccc (C[C@H](C([R])═ O)N(C)[R])cc1 |
| | | C₁₄H₁₉NO₂R₂ | |
| 282 | AEF(NMe3) | 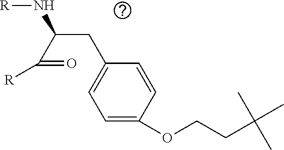 | C[N+](C)(C)CCO c1ccc(C[C@@H] (C([R])═O)N[R])cc1 |
| | | C₁₄H₂₁N₂O₂R₂⁺ | |
| 283 | 60(COCF₃)2Nal | 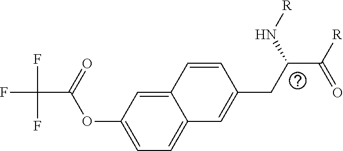 | O═C([C@H](Cc(c cc1c2)cc1ccc2OC (C(F)(F)F)═O)N[R]) [R] |
| | | C₁₅H₁₀F₃NO₃R₂ | |
| 284 | BIF | 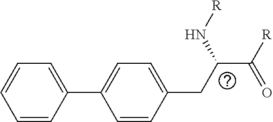 | O═C([C@H](Cc(c c1)ccc1- c1ccccc1)N[R])[R] |
| | | C₁₅H₁₃NOR₂ | |
| 285 | DiPhAla | 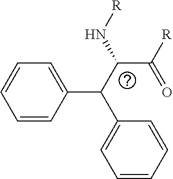 | O═C([C@H](C(c1 ccccc1)c1ccccc1) N[R])[R] |
| | | C₁₅H₁₃NOR₂ | |
| 286 | 5Et2Nal | 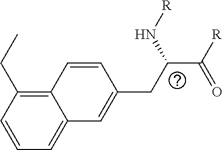 | CCc1c(ccc(C[C@ @H](C([R])═O)N [R])c2)c2ccc1 |
| | | C₁₅H₁₅NOR₂ | |
| 287 | CMF | 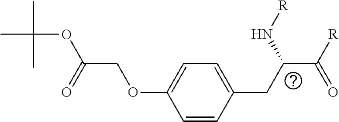 | CC(C)(C)OC(COc 1ccc(C[C@@H](C ([R])═O)N[R]) cc1)═O |
| | | C₁₅H₁₉NO₄R₂ | |
| 288 | F(4TzITMA1) | 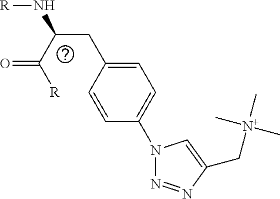 | C[N+](C)(C)Cc1c n(- c2ccc(C[C@@H] (C([R])═O)N[R])cc 2)nn1 |
| | | C₁₅H₂₀N₅OR₂⁺ | |
| 289 | PiperazinequatF | 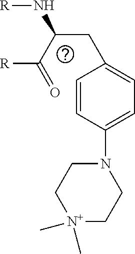 | C[N+](C)(CC1)C CN1c1ccc(C[C@ @H](C([R])═O)N [R])cc1 |
| | | C₁₅H₂₂N₃OR₂⁺ | |
| 290 | TMA3F | 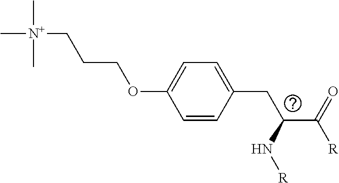 | C[N+](C)(C)CCC Oc1ccc(C[C@@H] (C([R])═O)N[R]) cc1 |
| | | C₁₅H₂₃N₂O₂R₂⁺ | |
| 291 | TMA4F | 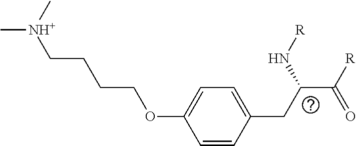 | C[NH+](C)CCCC Oc1ccc(C[C@@H] (C([R])═O)N[R]) cc1 |
| | | C₁₅H₂₃N₂O₂R₂⁺ | |
| 292 | K5cpa, K(5cpa), K(5cpaCO) | 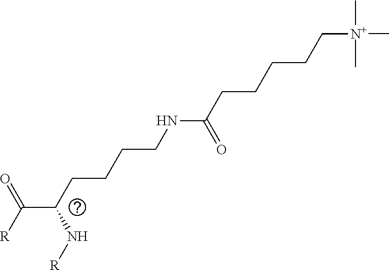 | C[N+](C)(C)CCC CCC(NCCCC[C@ @H](C([R])═O)N [R])═O |
| | | C₁₅H₃₀N₃O₂R₂⁺ | |
| 293 | dK(5cpa), k(5cpa), k(5cpaCO) | 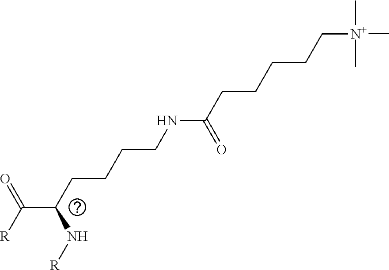 | C[N+](C)(C)CCC CCC(NCCCC[C@ H](C([R])═O)N [R])═O |
| | | C₁₅H₃₀N₃O₂R₂⁺ | |
| 294 | 2Nal6(3pyrazole) | 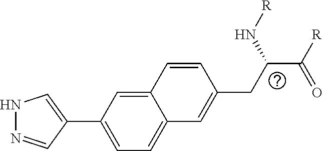 | O═C([C@H](Cc(c cc1c2)cc1ccc2- c1c[nH]nc1)N[R]) [R] |
| | | C₁₆H₁₃N₃OR₂ | |
| 295 | 7PyrTrp | 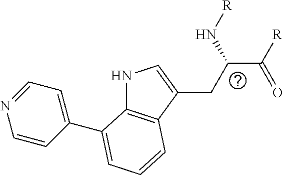 | O═C([C@H](Cc1c [nH]c2c1cccc2- c1ccncc1)N[R])[R]1 |
| | | C₁₆H₁₃N₃OR₂ | |
| 296 | 4BzF | 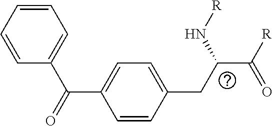 | O═C([C@H](Cc(c c1)ccc1C(c1ccccc 1)═O)N[R])[R] |
| | | C₁₆H₁₃NO₂R₂ | |
| 297 | aMeBiF | 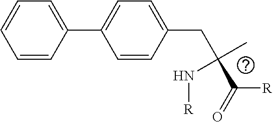 | C[C@](Cc(cc1)cc c1- c1ccccc1)(C([R])═ O)N[R] |
| | | C₁₆H₁₅NOR₂ | |
| 298 | NPyEF | 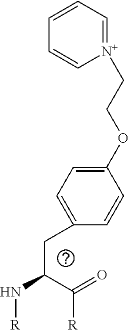 | O═C([C@H](Cc(c c1)ccc1OCC[n+]1 ccccc1)N[R])[R] |
| | | C₁₆H₁₇N₂O₂R₂⁺ | |
| 299 | 5iPr2Nal | 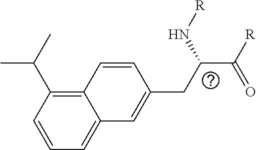 | CC(C)c1c(ccc(C[C @@H](C([R])═O) N[R])c2)c2ccc1 |
| | | C₁₆H₁₇NOR₂ | |
| 300 | TetraFAEF(Boc) | 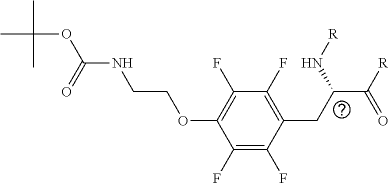 | CC(C)(C)OC(NC COc(c(F)c(c(C[C @@H](C([R])═O) N[R])c1F)F)c1F)═ O |
| | | C₁₆H₁₈F₄N₂O₄R₂ | |
| 301 | 4TMABYF | 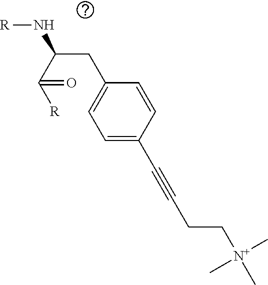 | C[N+](C)(C)CCC #Cc1ccc(C[C@@ H](C([R])═O)N[R]) cc1 |
| | | C₁₆H₂₁N₂OR₂⁺ | |
| 302 | AEF(Boc) | 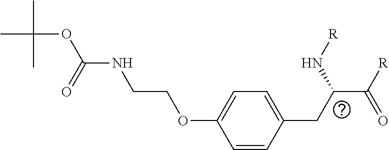 | CC(C)(C)OC(NC COc1ccc(C[C@@) H](C([R])═O)N[R]) cc1)═O |
| | | C₁₆H₂₂N₂O₄R₂ | |
| 303 | F(4TzlTMA2) | 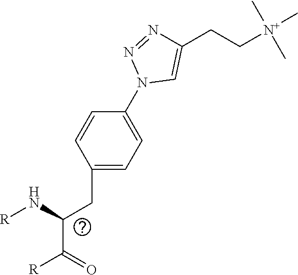 | C[N+](C)(C)CCc1 cn(-c2ccc(C[C@@H] (C([R])═O)N[R])cc 2)nn1 |
| | | C₁₆H₂₂N₅OR₂⁺ | |
| 304 | DMPMF | 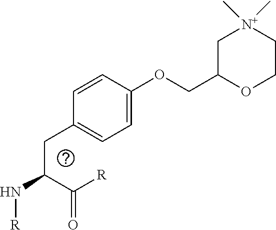 | C[N+]1(C)CC(CO c2ccc(C[C@@H]( C([R])═O)N[R])cc 2)OCC1 |
| | | C₁₆H₂₃N₂O₃R₂⁺ | |
| 305 | KDde, K(Dde) | 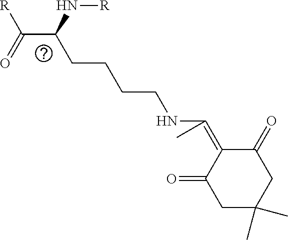 | CC(C)(CC(C1═C( C)NCCCC[C@@ H](C([R])═O)N[R])═ O)CC1═O |
| | | C₁₆H₂₄N₂O₃R₂ | |
| 306 | dKDde, k(Dde), dK(Dde) | 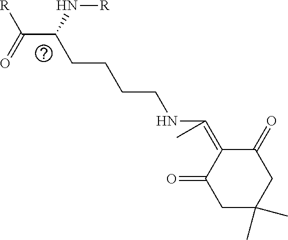 | CC(C)(CC(C1═C (C)NCCCC[C@H] (C([R])═O)N[R])═ O)CC1═O |
| | | C₁₆H₂₄N₂O₃R₂ | |
| 307 | Y(OEOXIMECh) | 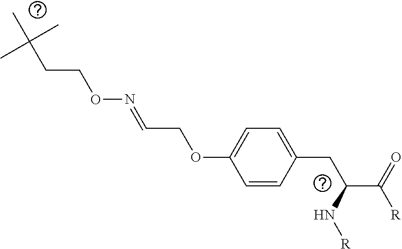 | C[N+](C)(C)CCO/ N═C/COc1ccc(C[ c@@H](C([R])═ O)N[R])cc1 |
| | | C₁₆H₂₄N₃O₃R₂⁺ | |
| 308 | Y(OZOXIMECh) | 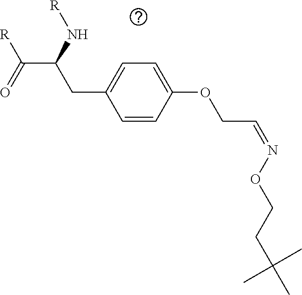 | C[N+](C)(C)CCO/ N═C\COc1ccc(C[ C@@H](C([R])═ O)N[R])cc1 |
| | | C₁₆H₂₄N₃O₃R₂⁺ | |
| 309 | AEF(NHCh) | 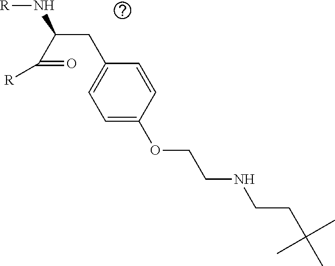 | C[N+](C)(C)CCN CCOc1ccc(C[C@ @H](C([R])═O)N[ R])cc1 |
| | | C₁₆H₂₆N₃O₂R₂⁺ | |
| 310 | K(Biotina), K(Biotin) | 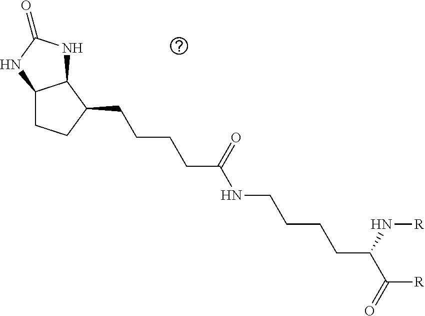 | O═C(CCCC[C@H] ([C@@H]1N2)S C[C@H]1NC2═O) NCCCC[C@@H] (C([R])═O)N[R] |
| | | C₁₆H₂₆N₄O₃SR₂ | |
| 311 | K(DAGSuc) | 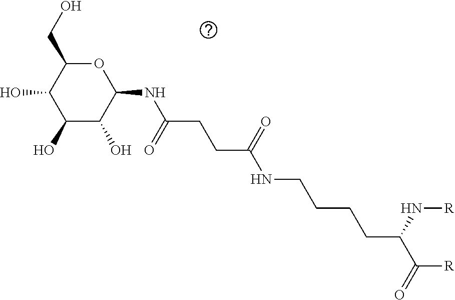 | OC[C@H]([C@H] ([C@@H]([C@H] 1O)O)O)O[C@H] 1NC(CCC(NCCC C[C@@H](C([R])═O) N[R])═O)═O |
| | | C₁₆H₂₇N₃O₈R₂ | |
| 312 | k(DAGSuc), dK(DAGSuc) | 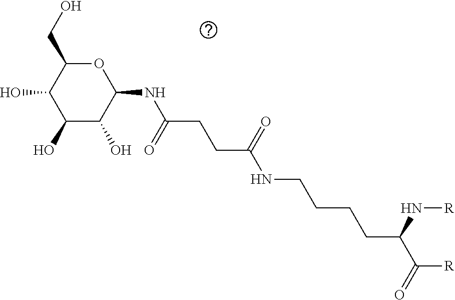 | OC[C@H]([C@H] ([C@@H]([C@H] 1O)O)O)O[C@H] 1NC(CCC(NCCC C[C@H](C([R])═ O)N[R])═O)═O |
| | | C₁₆H₂₇N₃O₈R₂ | |
| 313 | DOTA | 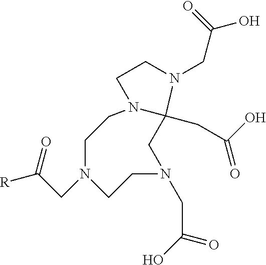 | OC(CN1CCN(CC (O)═O)CCN(CC([R 1)═O)CCN(CC (O)═O)CC1)═O |
| | | C₁₆H₂₇N₄O₇R | |
| 314 | Dab(NMeCarn) | 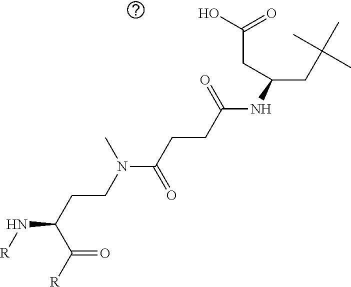 | CN(CC[C@@H] (C([R])═O)N[R])C (CCC(N[C@@H] (CC(O)═O)C[N+] (C)(C)C)═O)═O |
| | | C₁₆H₂₉N₄O₅R₂⁺ | |
| 315 | Dab(NMecarn) | 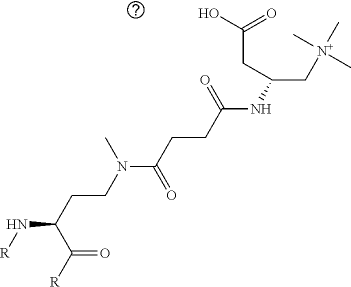 | CN(CC[C@@H] (C([R])═O)N[R])C (CCC(N[C@H](CC (O)═O)C[N+](C) (C)C)═O)═O |
| | | C₁₆H₂₉N₄O₅R₂⁺ | |
| 316 | orn(d) | 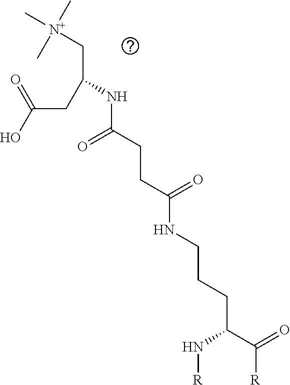 | C[N+](C)(C)C[C @@H](CC(O)═O) NC(CCC(NCCC [C@H](C([R])═O) N[R])═O)═O |
| | | C₁₆H₂₉N₄O₅R₂⁺ | |
| 317 | 2Nal6((5CF3)3 pyrazole) | 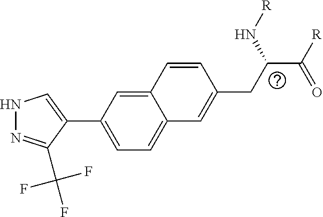 | O═C([C@H](Cc(c cc1c2)cc1ccc2- c1c[nH]nc1C(F)(F) F)N[R])[R] |
| | | C₁₇H₁₂F₃N₃OR₂ | |
| 318 | 7(2ClPh)W | 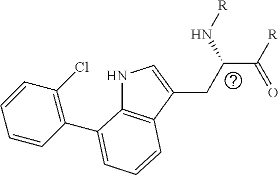 | O═C([C@H](Cc1c [nH]c2c1cccc2- c(cccc1)c1Cl)N[R]) [R] |
| | | C₁₇H₁₃ClN₂OR₂ | |
| 319 | TMAPF | | C[N+](C)(CCCCC |
| | | | Oc1ccc(C[C@H] |
| | | | (N[R])C([R])═O)cc |
| | | | 1)C |
| 320 | 7(2OMe5Pyr)W | 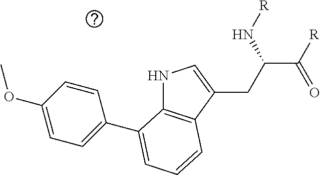 | COc(cc1)ncc1- c1cccc2c1[nH]cc2 C[C@@H](C([R])═O) N[R] |
| | | C₁₇H₁₅N₃O₂R₂ | |
| 321 | W-7Ph, 7-phenyl-L- tryptophan | 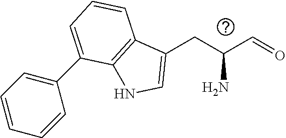 [SMILES: N[C@H](C=O)Cc1c[nH]c2c(-c3ccccc3)cccc12] | N[C@@H](Cc1c [nH]c2c1cccc2- c1ccccc1)C═O |
| | | C₁₇H₁₆N₂O | |
| 322 | 5OH2Nal | 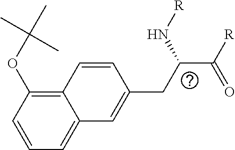 | CC(C)(C)Oc1c(cc c(C[C@@H](C([R])═ O)N[R])c2)c2c cc1 |
| | | (S)-2-amino-3-(5-(tert-butoxy)naphthalen-2- | |
| | | yl)propanoic acid | |
| | | C₁₇H₁₉NO₂R₂ | |
| 323 | 5tBu2Nal | 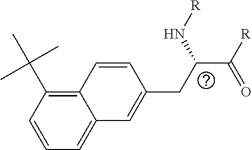 | CC(C)(C)c1c(ccc (C[C@@H](C([R])═O) N[R])c2)c2ccc 1 |
| | | C₁₇H₁₉NOR₂ | |
| 324 | hFTMAPF | 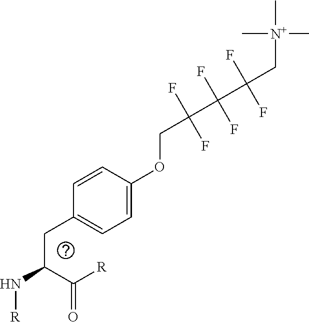 | C[N+](C)(C)CC(C (C(COc1ccc(C[C @@H\|(C([R])═O) N[R])cc1)(F)F)(F) F)(F)F |
| | | C₁₇H₂₁F₆N₂O₂R₂⁺ | |
| 325 | F(4TzlTMA3) | 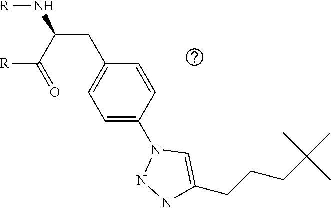 | C[N+](C)(C)CCCc 1cn(- c2ccc(C[C@@H] (C([R])═O)N[R])cc 2)nn1 |
| | | C₁₇H₂₄N₅OR₂⁺ | |
| 326 | DMMMF | 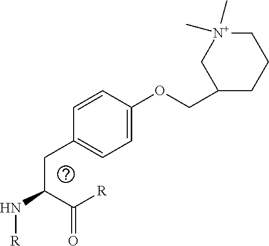 | C[N+]1(C)CC(CO c2ccc(C[C@@H] (C([R])═O)N[R])cc 2)CCC1 |
| | | C₁₇H₂₅N₂O₂R₂⁺ | |
| 327 | MMoEF | 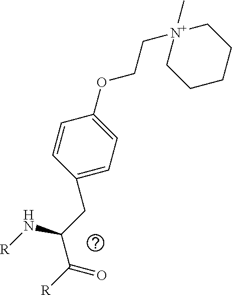 | C[N+]1(CCOc2cc c(C[C@@H](C([R])═ O)N[R])cc2)CC CCC1 |
| | | C₁₇H₂₅N₂O₂R₂⁺ | |
| 328 | MMoPF | 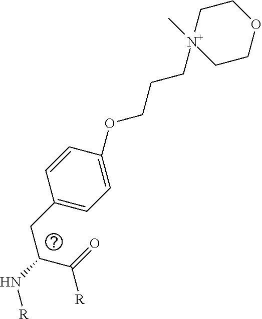 | C[N+]1(CCCOc2c cc(C[C@H](C([R])═ O)N[R])cc2)CC OCC1 |
| | | C₁₇H₂₅N₂O₃R₂⁺ | |
| 329 | AEF(MEP) | 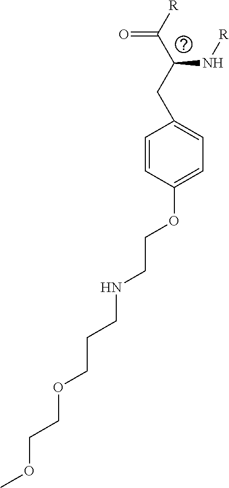 | COCCOCCCNCC Oc1ccc(C[C@@H] (C([R])═O)N[R]) cc1 |
| | | C₁₇H₂₆N₂O₄R₂ | |
| 330 | 4DMPzEF | 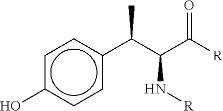 | C[N+]1(C)CCN(C COc2ccc(C[C@@ H](C([R])═O)N[R]) cc2)CC1 C[N+]1(C)CCN(C COc2ccc(C[C@@ H](C═O)N)cc2)CC1 |
| | | C₁₇H₂₆N₃O₂R₂⁺ | |
| 331 | TMAPF | 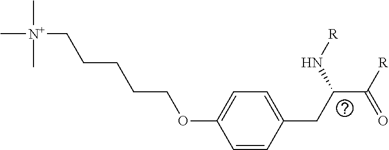 | C[N+](C)(C)CCC CCOc1ccc(C[C@ @H](C([R])═O) N[R])cc1 C[N+](C)(C)CCC CCOc1ccc(C[C@ @H\|(C═O)N)cc1 |
| | | C₁₇H₂₇N₂O₂R₂⁺ | |
| 332 | K(D), KCar | 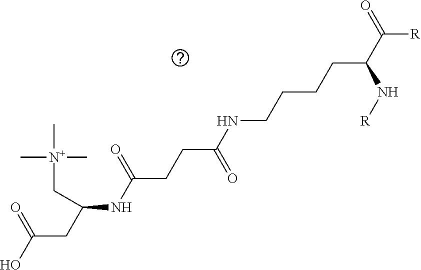 | C[N+](C)(C)C[C @H](CC(O)═O)N C(CCC(NCCCC[C @@H](C([R])═O) N[R])═O)═O |
| | | C₁₇H₃₁N₄O₅R₂⁺ | |
| 333 | K(d), KdCar | 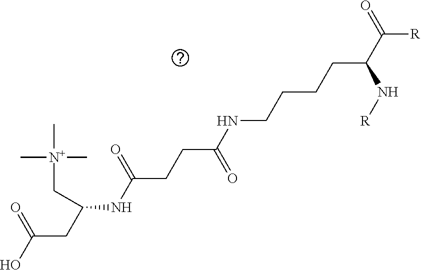 | C[N+](C)(C)C[C @@H\|(CC(O)═O) NC(CCC(NCCCC [C@@H](C([R])═ O)N[R])═O)═O |
| | | C₁₇H₃₁N₄O₅R₂⁺ | |
| 334 | k(D), dKCar | 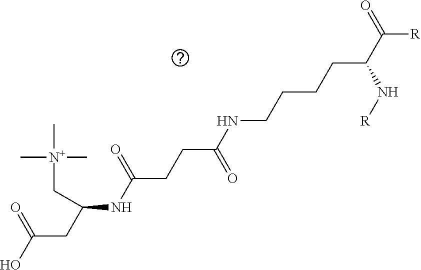 | C[N+](C)(C)C[C @H](CC(O)═O)N C(CCC(NCCCC[C @H](C([R])═O)N [R])═O)═O |
| | | C₁₇H₃₁N₄O₅R₂⁺ | |
| 335 | k(d), dKdCar | 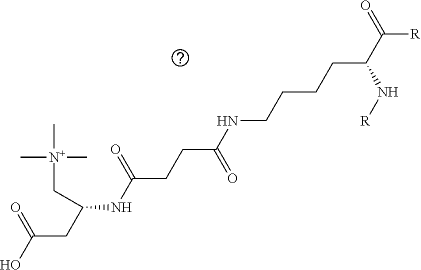 | C[N+](C)(C)C[C @@H](CC(O)═O) NC(CCC(NCCCC [C@H](C([R])═O) N[R])═O)═O |
| | | C₁₇H₃₁N₄O₅R₂⁺ | |
| 336 | 7(3CF₃TAZP)W | 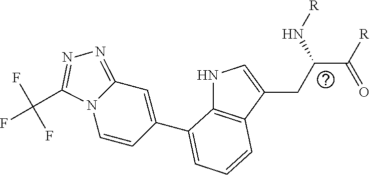 | O═C([C@H](Cc1c [nH]c2c1cccc2- c1cc2nnc(C(F)(F) F)n2cc1)N[R])[R] |
| | | C₁₈H₁₂F₃N₅OR₂ | |
| 337 | 7(4OCF₃Ph)W | 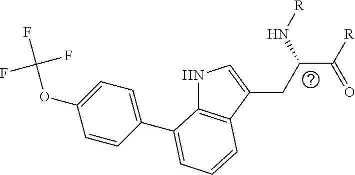 | O═C([C@H](Cc1c [nH]c2c1cccc2- c(cc1)ccc1OC(F) (F)F)N[R])[R] |
| | | C₁₈H₁₃F₃N₂O₂R₂ | |
| 338 | 7(4CF₃Ph)W | 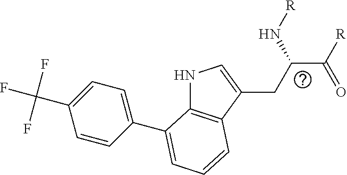 | O═C([C@H](Cc1c [nH]c2c1cccc2- c1ccc(C(F)(F)F)cc 1)N[R])[R] |
| | | C₁₈H₁₃F₃N₂OR₂ | |
| 339 | 7(7ImidPyr)W | 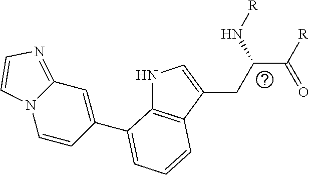 | O═C([C@H](Cc1c [nH]c2c1cccc2- c1cc2nccn2cc1)N [R])[R] |
| | | C₁₈H₁₄N₄OR₂ | |
| 340 | Y(C₉OH) | 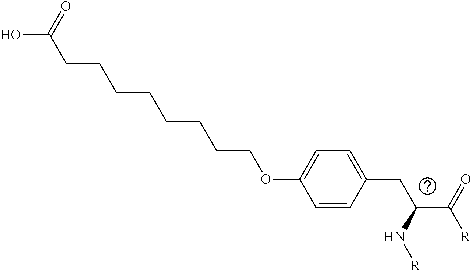 | OC(CCCCCCCC Oc1ccc(C[C@@H] (C([R])═O)N[R]) cc1)═O |
| | | C₁₈H₂₅NO₄R₂ | |
| 341 | Y(OTzlCh) | 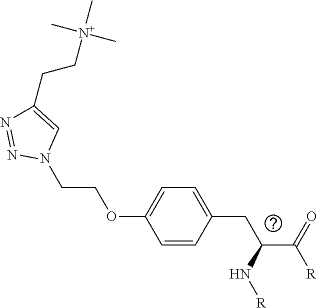 | C[N+](C)(C)CCc1 cn(CCOc2ccc(C[C @@H](C([R])═O) N[R])cc2)nn1 |
| | | C₁₈H₂₆N₅O₂R₂⁺ | |
| 342 | 4DMPEF | 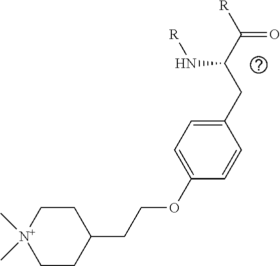 | C[N+]1(C)CCC(C COc2ccc(C[C@@ H](C([R])═O)N[R]) cc2)CC1 C[N+]1(C)CCC(C COc2ccc(C[C@@ H](C═O)N)cc2)C C1 |
| | | C₁₈H₂₇N₂O₂R₂⁺ | |
| 343 | AEF(AcCh) | 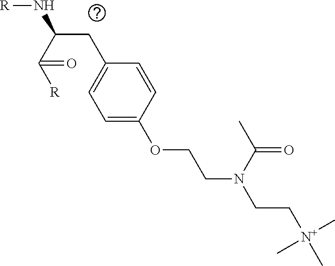 | CC(N(CC[N+](C) (C)C)CCOc1ccc(C [C@@H](C([R])═ O)N[R])cc1)═O |
| | | C₁₈H₂₈N₃O₃R₂⁺ | |
| 344 | TMA6F | 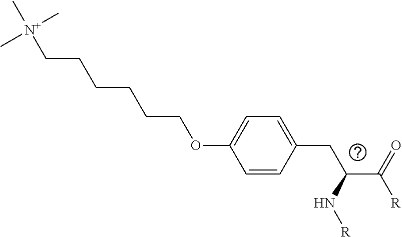 | C[N+](C)(C)CCC CCCOc1ccc(C[C N[R])cc1 |
| | | C₁₈H₂₉N₂O₂R₂⁺ | |
| 345 | AEF(MePrpa) | 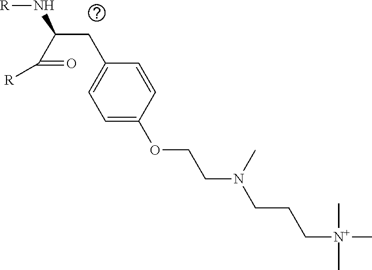 | CN(CCC[N+](C) (C)C)CCOc1ccc(C [C@@H](C([R])═ O)N[R])cc1 |
| | | C₁₈H₃₀N₃O₂R₂⁺ | |
| 346 | 2Nal6(Ph2OH) | 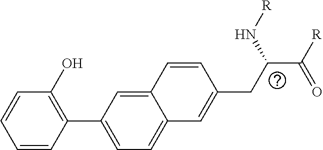 | Oc(cccc1)c1- c1ccc(cc(C[C@@ H](C([R])═O)N[R]) cc2)c2c1 |
| | | C₁₉H₁₅NO₂R₂ | |
| 347 | 7(3NAcPh)W | 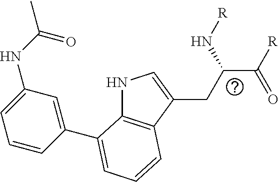 | CC(Nc1cccc (-c2cccc3c2[nH]cc3 C[C@@H](C([R])═O) N[R])c1)═O |
| | | C₁₉H₁₇N₃O₂R₂ | |
| 348 | 7(4NAcPh)W | 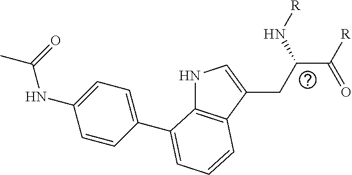 | CC(Nc(cc1)ccc1- c1cccc2c1[nH]cc2 C[C@@H](C ([R])═O)N[R])═O |
| | | C₁₉H₁₇N₃O₂R₂ | |
| 349 | 4PipF, 4PipPhe | C₁₉H₂₆N₂O₃R₂ | CC(C)(C)OC(N(C |
| | | | C1)CCClc1ccc(C |
| | | | [C@@H](C([R])═ |
| | | | O)N[R])cc1)═O |
| 350 | a | 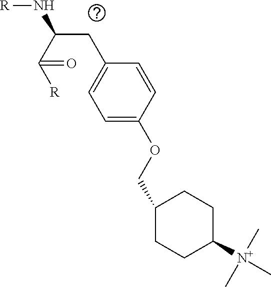 | C[N+](C)(C)[C@ H]1CC[C@H](CO c2ccc(C[C@@H] (C([R])═O)N[R])cc 2)CC1 |
| | | C₁₉H₂₉N₂O₂R₂⁺ | |

TABLE 2D
Peg monomers and Peg conjugated monomers.

| # | Structure | Names and Synonyms | Smiles Structure |
|---|---|---|---|
| 1 | C7H15NO3 | CON(MePEG2) | CN(CCOCCOC)C═O |
| 2 | C7H14O4 | mPEG3CO | COCCOCCOCC═O |
| 3 | C14H28O7 | mPEG6CO | COCCOCCOCCO |
| | | | CCOCCOCCC═O |
| 4 | C21H36N3O5+ | AEFNMePEG3a, | C[N+](C)(C)CCOC |
| | | AEF(NHcPEG3a) | COCCC(NCCOc1c |
| | | | cc(C[C@@H](C═O) |
| | | | N)cc1)═O |
| 5 | 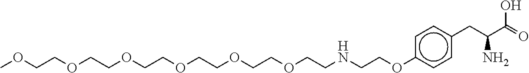 | AEFNmPEG6, AEF(NmPEG6) | COCCOCCOCCO CCOCCOCCNCC Oc1ccc(C[C@@H] (C═O)N)cc1 |
| | C24H42N2O8 | | |
| 6 | 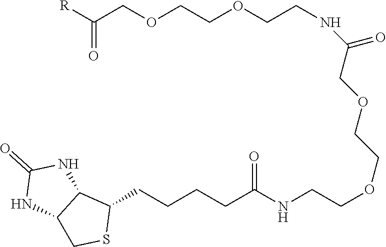 | BiotinPEG2PEG2, Biotin(PEG2PEG2) | O═C(CCCC[C@@ H]([C@H]1N2)SC [C@@H]1NC2═O) NCCOCCOCC(NC COCCOCC ([R])═O)═O |
| | C22H37N4O8SR | | |
| 7 | 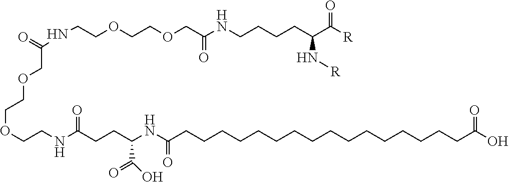 | K (PEG2PEG2gEC18OH) | OC(CCCCCCCCC CCCCCCCC(N [C@@H](CCC(NCC OCCOCC(NCCOC COCC(NCCCC [CN[R])═O)═O)═O) C(O)═O)═O)═O |
| | C41H73N5O13R2 | | |
| 8 | 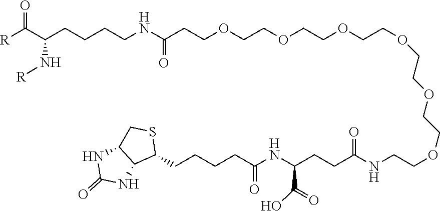 | K(PEG6gEBiotin) | OC([C@H](CCC (NCCOCCOCCOCC OCCOCCOCCC (NCCCC[C@@H](C ([R])═O)N[R])═O) ═O)NC(CCCC [C@H] ([C@@H]1N2)SC [C@H] 1NC2═O)═O)═O |
| | C36H62N6O13SR2 | | |
| 9 | 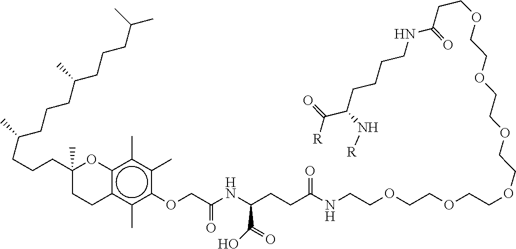 | K(PEG6gEVitE) | CC(C)CCC[C@@H] (C)CCC[C@@H] (C)CCC[C@](C) (CC1)Oc(c(C)c2C) c1c(C)c2OCC(N [C@@H](CCC(NCC OCCOCCOCCOC COCCOCCC(NCC CC[C@@H](C ([R])═O) N[R])═O)═O) C(O)═O)═O |
| | C57H98N4O14R2 | | |
| 10 | 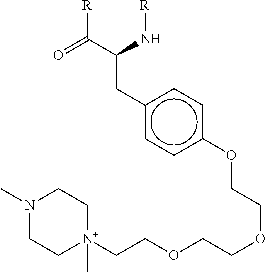 | MPzPEG3F | CN1CC[N+](C) (CCOCCOCCOc2ccc (C[C@@H](C([R]) ═O)N[R])cc2)CC1 |
| | C21H34N3O4R2+ | | |
| 11 | 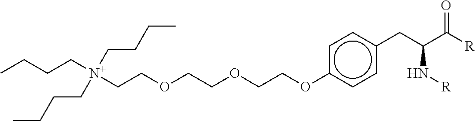 | TBAPEG3F | CCCC[N+](CCCC) (CCCC)CCOCCO CCOc1ccc (C[C@@H] (C([R])═O)N [R])cc1 |
| | C27H47N2O4R2+ | | |
| 12 | 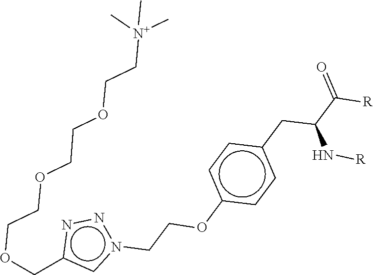 | Y(OTzlPEG3a) | C[N+](C)(C)CCOC COCCOCc1cn(CC Oc2ccc(C[C@@H] (C([R])═O)N[R])cc 2)nn1 |
| | C23H36N5O5R2+ | | |
| 13 | 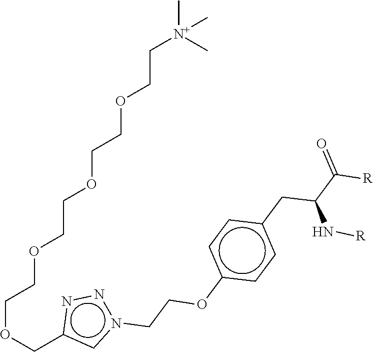 | Y(OTzlPEG4a) | C[N+](C)(C)CCOC COCCOCCOCc1cn (CCOc2ccc (C[C@@H] (C([R])═O)N [R])cc2)nn1 |
| | C25H40N5O6R2+ | | |
| 14 | 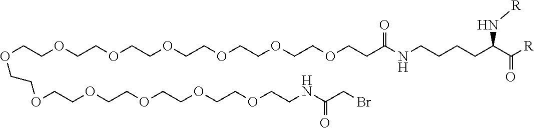 | k(PEG6Biotin), dK(PEG6Biotin) | O═C(CCOCCOCC OCCOCCOCCOC COCCOCCOCCO CCOCCOCCNC(C Br)═O)NCCCC[C @H](C([R])═O)N [R] |
| | C35H66BrN3O15R2 | | |
| 15 | 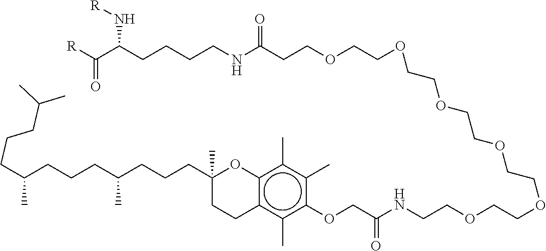 | k(dPEG12Ac), dK(dPEG12Ac) | CC(C)CCC[C@@ H](C)CCC[C@@H] (C)CCC[C@](C) (CC1)Oc(c(C)c2C)c 1c(C)c2OCC(NCC OCCOCCOCCOC COCCOCCC(NCC CC[C@H](C ([R])═O)N[R]) ═O)═O |
| | C52H91N3O11R2 | | |
| 16 | 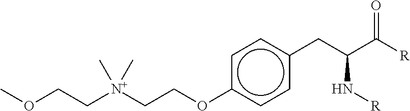 | mPEG2TMA2F | C[N+](C)(CCOC)C COc1ccc(C[C@@ H](C([R])═O)N[R]) cc1 |
| | C16H25N2O3R2+ | | |
| 17 | 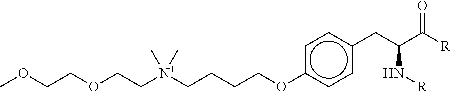 | mPEG3TMA4F | C[N+](C)(CCCCO c1ccc(C[C@@H] (C([R])═O)N[R]) cc1)CCOCCOC |
| | C20H33N2O4R2+ | | |
| 18 | 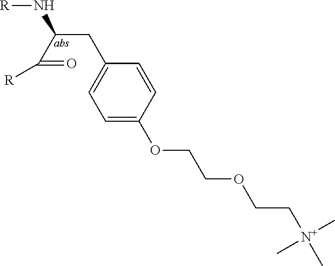 | | C[N+](C)(C) CCOCCO c1ccc(C[C@@H](C ([R])═O)N[R])cc1 |
| | C16H25N2O3R2+ | | |
| 19 | 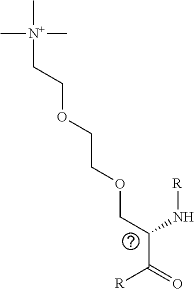 | | C[N+](C)(C) CCOCCOC [C@@H] (C([R])═O) N[R] |
| | C10H21N2O3R2+ | | |
| 20 | 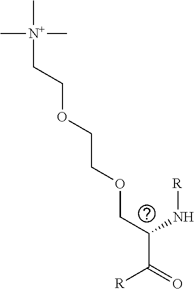 | | C[N+](C)(C) CCOCCOCC (C([R])═O)N[R] |
| | C10H21N2O3R2+ | | |
| 21 | 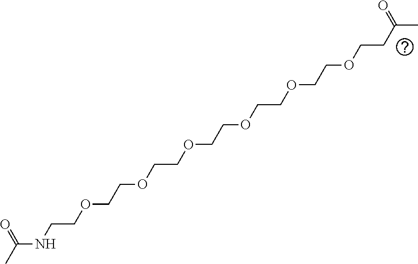 | | CC(NCCOC COCCOCCO CCOCCOCCC ([R])═O)═O |
| | C17H32NO8R | | |
| 22 | 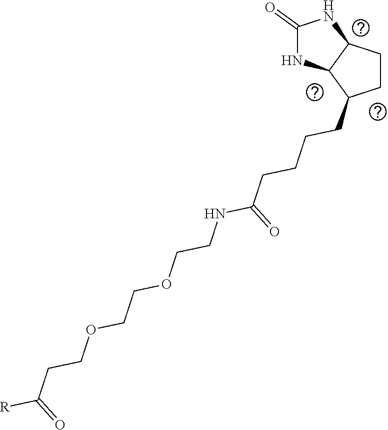 | | O═C(CCCC [C@@H] ([C@H]1N2)SC [C@@H] 1NC2═O) NCCOCCO CCC([R])═O |
| | C17H28N3O5SR | | |
| 23 | 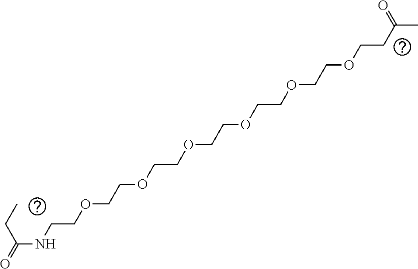 | | O═C(CBr) NCCOCCOCCO CCOCCOCCOCC C([R])═O |
| | C17H31BrNO8R | | |
| 24 | 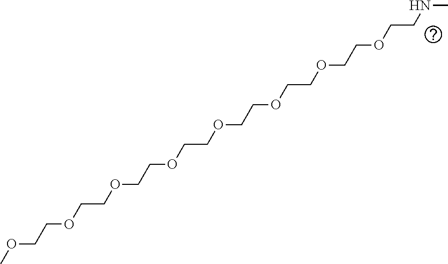 | | COCCOCCO CCOCCOCCO CCOCCOCCN[R] |
| | C17H36NO8R | | |
| 25 | 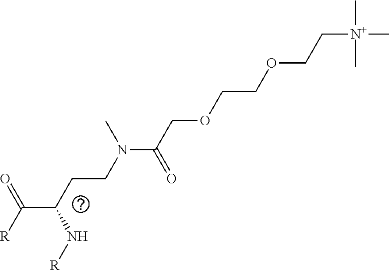 | | CN(CC[C@@H] (C([R])═O)N[R]) C(COCCOC C[N+](C)(C)C)═O |
| | C14H28N3O4R2+ | | |
| 26 | 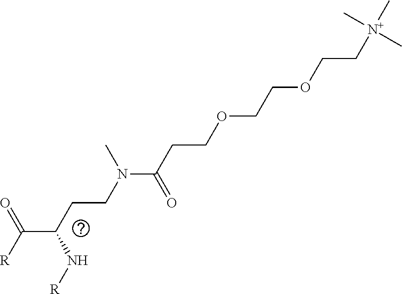 | | CN(CC[C@@H] (C([R])═O)N[R]) C(CCOCCO CC[N+] (C)(C)C)═O |
| | C15H30N3O4R2+ | | |
| 27 | 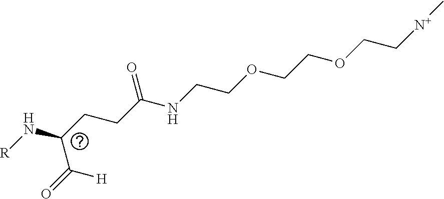 | | C[N+](C)(C) CCOCCOCCNC (CC[C@@H](C ([R])═O)N[R])═O |
| | C14H28N3O4R2+ | | |
| 28 | 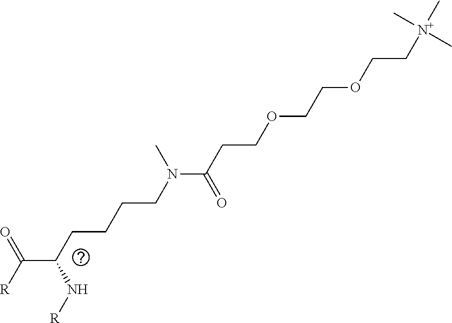 | | CN(CCCC[C@@H] (C([R])═O) N[R])C(CCOC COCC[N+] (C)(C)C)═O |
| | C17H34N3O4R2+ | | |
| 29 | 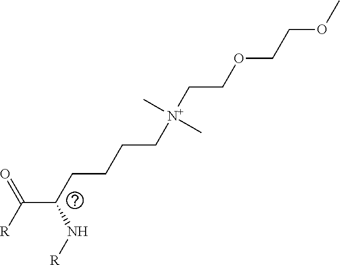 | | C[N+](C)(CCCC [C@@H](C ([R])═O)N[R]) CCOCCOC |
| | C13H27N2O3R2+ | | |
| 30 | 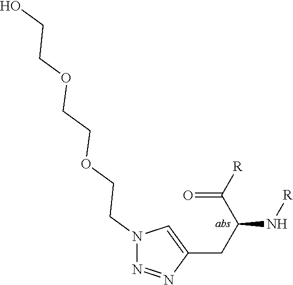 | | OCCOCCOCC n1nnc (C[C@@H] (C([R])═O)N [R])c1 |
| | C11H18N4O4R2 | | |
| 31 | 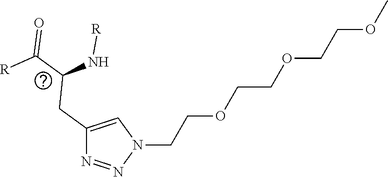 | | COCCOCCOCC n1nnc(C[C@@H] (C([R])═O) N[R])c1 |
| | C12H20N4O4R2 | | |
| 32 | 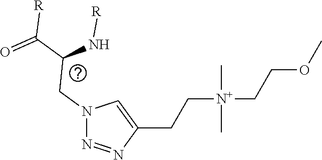 | | C[N+](C)(CCc1cn (C[C@@H] (C([R])═O)N[R]) nn1)CCOC |
| | C12H22N5O2R2+ | | |
| 33 | 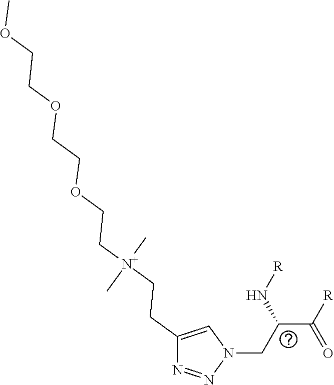 | | C[N+](C)(CCc1cn (C[C@@H] (C([R])═O)N[R]) nn1) CCOCCOCCOC |
| | C16H30N5O4R2+ | | |
| 34 | 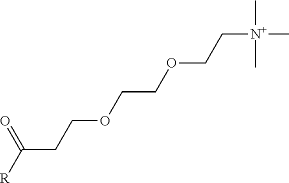 | | C[N+](C)(C) CCOCCO CCC([R])═O |
| | C10H21NO3R+ | | |
| 35 | 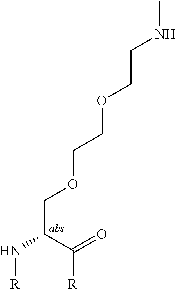 | | CNCCOCCOC [C@H] (C([R])═O)N[R] |
| | C8H16N2O3R2 | | |
| 36 | 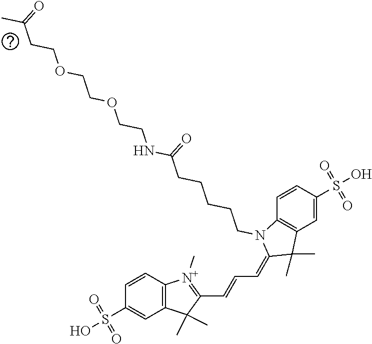 | (SulfoCy3dPEG2) | CC1(C)c(cc(cc2)S (O)(═O)═O)c2[N+] (C)═C1/C═C/C═C (/C1(C)C)\N(CCCC CC(NCCOCCOCC C([R])═O)═O)c(cc2) c1cc2S(O)(═O)═O |
| | C37H49N3O10S2R+ | | |
| 37 | 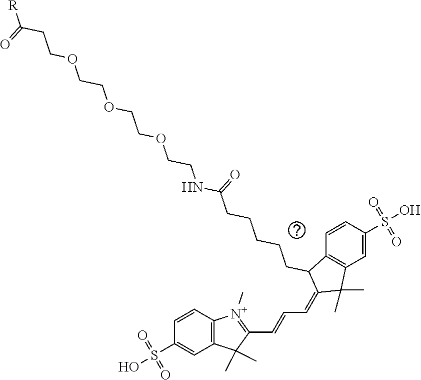 | (SulfoCy3dPEG3) | CC1(C)c(cc(cc2)S (O)(═O)═O)c2[N+] (C)═C1/C═C/ C═C(/C1(C) C)\N(CCCCCC (NCCOCCOCC OCCC([R])═O)═O) c(cc2)c1cc2S(O) (═O)═O |
| | C39H53N3O11S2R+ | | |
| 38 | 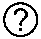 | (d)gEPEG2PEG2 | C[N+](C)(C)C[C@ @H](CC(O)═O)NC |
| | C28H49N5O13R+ | | (CCC(N[C@@H] |
| | | | (CCC(NCCOCCOC |
| | | | C(NCCOCCOCC |
| | | | ([R])═O)═O)═O)C |
| | | | (O)═O)═O)═O |
| 39 | 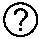 | AcdPEG12CO | CC(NCCOCCOCC OCCOCCOCCOC |
| | C29H57NO14 | | COCCOCCOCCO |
| | | | CCOCCOCCC═O) |
| | | | ═O |
| 40 | 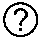 | AcdPEG9CO | CC(NCCOCCOCC OCCOCCOCCOC |
| | C23H45NO11 | | COCCOCCOCCC═ |
| | | | O)═O |
| 41 | 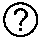 | AEEP(PEG2PEG2g EC18OH) | OC(CCCCCCCCC CCCCCCCC(N[C |
| | C42H75N4O15R | | @@H](CCC(NCC |
| | | | OCCOCC(NCCOC |
| | | | COCC(NCCOCCO |
| | | | CCC([R])═O)═O)═ |
| | | | O)═O)C(O)═O)═O) |
| | | | ═O |
| 42 | 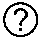 | AEEPPEG2PEG2gEC1 8OH, | C[N+](C)(CCNC(C OCCOCCNC(CC |
| | C47H86N7O14R2+ | k(PEG2Sp6PEG2gEC1 | [C@@H](C(O)═O) |
| | | 8OH), | NC(CCCCCCCCC |
| | | dK(PEG2Sp6PEG2gEC | CCCCCCCC(O)═O) |
| | | 18OH) | ═O)═O)═O)CC(N |
| | | | CCOCCOCC(NCC |
| | | | CC[C@H](C([R])═ |
| | | | O)N[R])═O)═O |
| 43 | 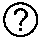 | AEF((Ch)cPEG3a) | C[N+](C)(C)CCN (CCOc1ccc(C[C@ |
| | C26H46N4O5R2+2 | | @H](C([R])═O)N |
| | | | [R])cc1)C(CCOCC |
| | | | OCC[N+](C)(C)C) |
| | | | ═O |
| 44 | 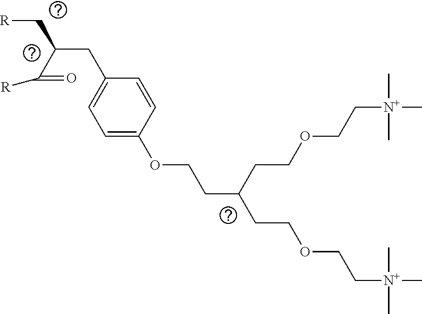 | AEF(BisPEG2a)(RS) AEF(BisPEG2a)(S*) (The RS and the S* indicates the stereochemistry) | C[N+](C)(C)CCOC CN(CCOCC[N+](C) (C)C)CCOc1ccc(C C(C([R])═O)N[R]) cc1 |
| | C25H46N4O4R2+2 | | |
| 45 | 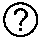 | AEF(NMePEG3a), AEF(NMecPEG3aCO) | C[N+](C)(C)CCOC COCCC(NCCOc1c |
| | C21H34N3O5R2+ | | cc(C[C@@H](C |
| | | | ([R])═O)N[R]) |
| | | | cc1)═O |
| 46 | 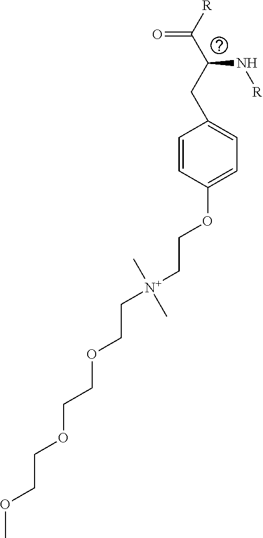 | AEF(NMe2mPEG3) | C[N+](C)(CCOCC OCCOC)CCOc1cc c(C[C@@H](C ([R])═O)N[R])cc1 |
| | C20H33N2O5R2+ | | |
| 47 | 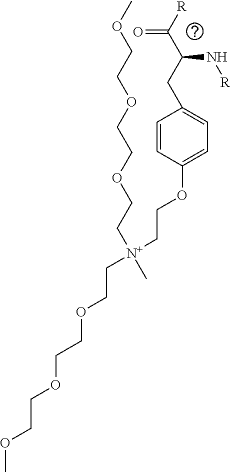 | AEF(NMeBismPEG3) | C[N+](CCOCCOC COC)(CCOCCOC COC)CCOc1ccc(C [C@@H](C([R])═O) N[R])cc1 |
| | C26H45N2O8R2+ | | |
| 48 | 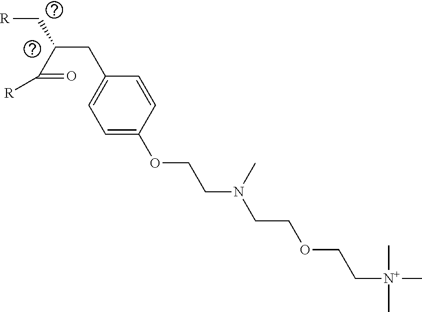 | AEF(NMePEG2a) | CN(CCOCC[N+](C) (C)C)CCOc1ccc(C [C@H](C([R])═O) N[R])cc1 |
| | C19H32N3O3R2+ | | |
| 49 | 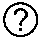 | AEF(NmPEG6) | COCCOCCOCCO CCOCCOCCNCC |
| | C24H40N2O8R2 | | Oc1ccc(C[C@@H] |
| | | | (C([R])═O)N[R])cc1 |
| 50 | 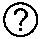 | AEF(PEG2PEG2g EC16OH) | OC(CCCCCCCCC CCCCCC(N[C@@ |
| | C44H71N5O14R2 | | H](CCC(NCCOCC |
| | | | OCC(NCCOCCOC |
| | | | C(NCCOc1ccc(C[C |
| | | | @@H](C([R])═O) |
| | | | N[R])cc1)═O)═O) |
| | | | ═O)C(O)═O)═O)═O |
| 51 | 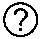 | AEF(PEG2PEG2g EC18OH) | OC(CCCCCCCCC CCCCCCCC(N[C |
| | C46H75N5O14R2 | | @@H](CCC(NCC |
| | | | OCCOCC(NCCOC |
| | | | COCC(NCCOc1cc |
| | | | c(C[C@@H](C([R]) |
| | | | ═O)N[R])cc1)═O) |
| | | | ═O)═O)C(O)═O) |
| | | | ═O)═O |
| 52 | 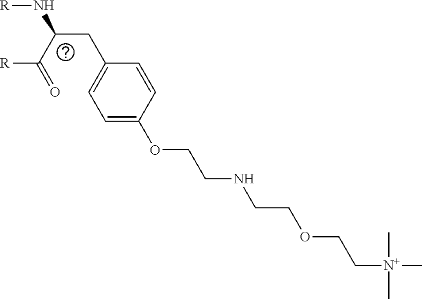 | AEF(Peg2a), AEF(PEG2a) | C[N+](C)(C)CCOC CNCCOc1ccc(C[C @@H](C([R])═O) N[R])cc1 |
| | C18H30N3O3R2+ | | |
| 53 | 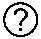 | AEF(SP6PEG12g EC18OH) | C[N+](C)(CCNC(C COCCOCCOCCO |
| | C67H119N6O22R2+ | | CCOCCOCCOCC |
| | | | OCCOCCOCCOC |
| | | | COCCNC(CC[C@ |
| | | | @H](C(O)═O)NC |
| | | | (CCCCCCCCCCCC |
| | | | CCCCC(O)═O)═O) |
| | | | ═O)═O)CC(NCCO |
| | | | c1ccc(C[C@@H](C |
| | | | ([R])═O)N[R]) |
| | | | cc1)═O |
| 54 | 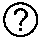 | AEF(SP6PEG12g EC20OH) | C[N+](C)(CCNC(C COCCOCCOCCO |
| | C69H123N6O22R2+ | | CCOCCOCCOCC |
| | | | OCCOCCOCCOC |
| | | | COCCNC(CC[C@ |
| | | | @H](C(O)═O)NC |
| | | | (CCCCCCCCCCCC |
| | | | CCCCCCC(O)═O) |
| | | | ═O)═O)═O)CC(NC |
| | | | COc1ccc(C[C@@ |
| | | | H](C([R])═O)N[R]) |
| | | | cc1)═O |
| 55 | 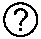 | AEF(SP6PEG2PEG2g EC18OH) | C[N+](C)(CCNC(C OCCOCCNC(COC |
| | C52H88N7O15R2+ | | COCCNC(CC[C@ |
| | | | @H](C(O)═O)NC |
| | | | (CCCCCCCCCCCC |
| | | | CCCCC(O)═O)═O) |
| | | | ═O)═O)═O)CC(NC |
| | | | COc1ccc(C[C@@ |
| | | | H](C([R])═O)N[R]) |
| | | | cc1)═O |
| 56 | 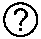 | AEF(SP6PEG2PEG2g EC20OH) | C[N+](C)(CCNC(C OCCOCCNC(COC |
| | C54H92N7O15R2+ | | COCCNC(CC[C@ |
| | | | @H](C(O)═O)NC |
| | | | (CCCCCCCCCCCC |
| | | | CCCCCCC(O)═O) |
| | | | ═O)═O)═O)═O)CC |
| | | | (NCCOc1ccc(C[C |
| | | | @@H](C([R])═O) |
| | | | N[R])cc1)═O |
| 57 | C55H95N6O16R2+ | AEF(SP6PEG6gEC18 | C[N+](C)(CCNC(C |
| | | OH) | COCCOCCOCCO |
| | | | CCOCCOCCNC(C |
| | | | C[C@@H](C(O)═ |
| | | | O)NC(CCCCCCC |
| | | | CCCCCCCCCC(O) |
| | | | ═O)═O)═O)═O)CC |
| | | | (NCCOc1ccc(C[C |
| | | | @@H](C([R])═O) |
| | | | N[R])cc1)═O |
| 58 | C57H99N6O16R2+ | AEF(SP6PEG6gEC20 | C[N+](C)(CCNC(C |
| | | OH) | COCCOCCOCCO |
| | | | CCOCCOCCNC(C |
| | | | C[C@@H](C(O)═ |
| | | | O)NC(CCCCCCC |
| | | | CCCCCCCCCCCC |
| | | | (O)═O)═O)═O)═O) |
| | | | CC(NCCOc1ccc(C |
| | | | [C@@H](C([R])═O) |
| | | | N[R])cc1)═O |
| 59 | 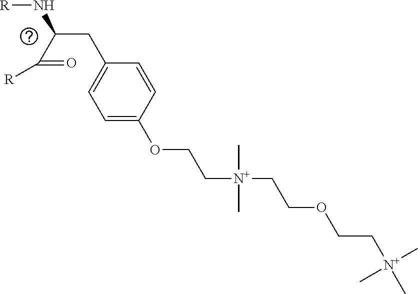 | AEF(aPEG2a) | C[N+](C)(C)CCOC C[N+](C)(C)CCOc 1ccc(C[C@@H](C ([R])═O)N[R])cc1 |
| | C20H35N3O3R2+2 | | |
| 60 | 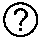 | k(PEG2gEC18OH), dK(PEG2gEC18OH) | OC(CCCCCCCCC CCCCCCCC(N[C |
| | C35H62N4O10R2 | | @@H](CCC(NCC |
| | | | OCCOCC(NCCCC |
| | | | [C@H](C([R])═O) |
| | | | N[R])═O)═O)C(O) |
| | | | ═O)═O)═O |
| 61 | 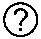 | k(PEG6gEC18OH), dK(PEG6gEC18OH) | OC(CCCCCCCCC CCCCCCCC(N[C |
| | C44H80N4O14R2 | | @@H](CCC(NCC |
| | | | OCCOCCOCCOC |
| | | | COCCOCCC(NCC |
| | | | CC[C@H](C([R]) |
| | | | ═O)N[R])═O)═O) |
| | | | C(O)═O)═O)═O |
| 62 | 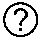 | ,k(Sp6PEG2PEG2g EC18OH), | C[N+](C)(CCNC(C OCCOCCNC(COC |
| | C47H86N7O14R2+ | dK(Sp6PEG2PEG2gEC | COCCNC(CC[C@ |
| | | 18OH) | @H](C(O)═O)NC |
| | | | (CCCCCCCCCCCC |
| | | | CCCCC(O)═O)═O) |
| | | | ═O)═O)═O)CC(NC |
| | | | CCC[C@H](C([R]) |
| | | | ═O)N[R])═O |
| 63 | 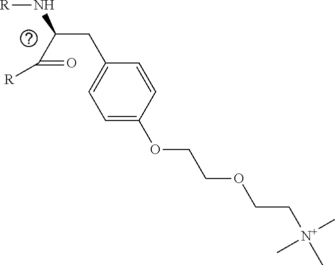 | APEG2F | C[N+](C)(C)CCOC COc1ccc(C[C@@ H](C([R])═O)N[R]) cc1 |
| | C16H25N2O3R2+ | | |
| 64 | 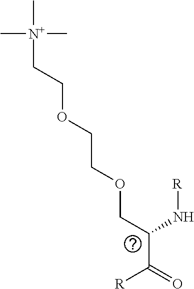 | APEG2ser | C[N+](C)(C)CCOC COC[C@@H](C ([R])═O)N[R] |
| | C10H21N2O3R2+ | | |
| 65 | 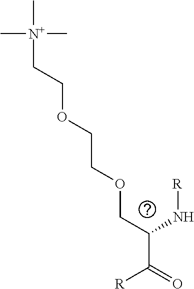 | APEG2Ser(R*) APEG2Ser(S*) | C[N+](C)(C)CCOC COCC(C([R])═O)N [R] |
| | C10H21N2O3R2+ | | |
| 66 | 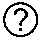 | APEG3F | C[N+](C)(C)CCOC COCCOc1ccc(C[C |
| | C18H29N2O4R2+ | | @@H](C([R])═O) |
| | | | N[R])cc1C |
| | | | [N+](C)(C)CCOC |
| | | | COCCOc1ccc(C |
| | | | [C@@H](C═O) |
| | | | N)cc1 |
| 67 | 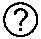 | AcdPEG6CO | CC(NCCOCCOCC OCCOCCOCCOC |
| | C17H32NO8R | | CC([R])═O)═O |
| 68 | 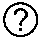 | BiotinPEG4CO, Biotin(PEG4CO), | O═C(CCCC[C@@ H]([C@H]1N2)SC |
| | C21H36N3O7SR | Biotin(PEG4) | [C@@H]1NC2═O) |
| | | | NCCOCCOCCOC |
| | | | COCCC([R])═O |
| 69 | 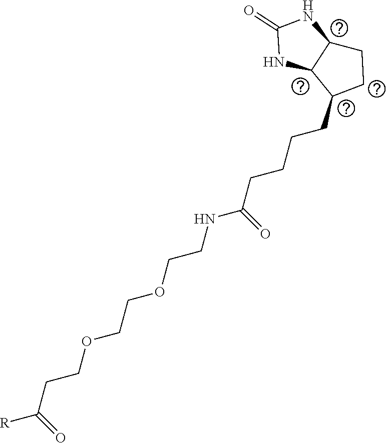 | Biotinyl(dPEG2), Biotin(dPEG2) | O═C(CCCC[C@@ H]([C@H]1N2)SC [C@@H]1NC2═O) NCCOCCOCCC ([R])═O |
| | C17H28N3O5SR | | |
| 70 | 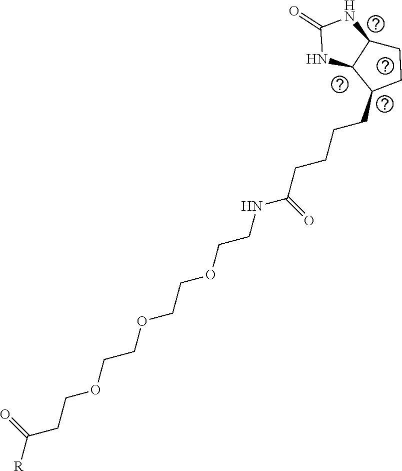 | Biotinyl(dPEG3), Biotin(dPEG3) | O═C(CCCC[C@@ H]([C@H]1N2)SC [C@@H]1NC2═O) NCCOCCOCCOC CC([R])═O |
| | C19H32N3O6SR | | |
| 71 | 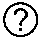 | BrAcdPEG12CO | O═C(CBr)NCCOC COCCOCCOCCO |
| | C29H55BrNO14R | | CCOCCOCCOCC |
| | | | OCCOCCOCCOC |
| | | | CC([R])═O |
| 72 | 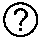 | BrAcdPEG6CO | O═C(CBr)NCCOC COCCOCCOCCO |
| | C17H31BrNO8R | | CCOCCC([R])═O |
| 73 | 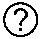 | BrAcdPEG9CO | O═C(CBr)NCCOC COCCOCCOCCO |
| | C23H43BrNO11R | | CCOCCOCCOCC |
| | | | OCCC([R])═O |
| 74 | 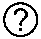 | C12gEPEG2PEG2, C12gEPEG2PEG2CO | CCCCCCCCCCCC (N[C@@H](CCC |
| | C29H52N3O10R | | (NCCOCCOCC(NC |
| | | | COCCOCC([R])═O) |
| | | | ═O)═O)C(O) |
| | | | ═O)═O |
| 75 | 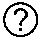 | C14gEPEG2PEG2, C14gEPEG2PEG2CO | CCCCCCCCCCCC CC(N[C@@H](CC |
| | C31H56N3O10R | | C(NCCOCCOCC |
| | | | (NCCOCCOCC([R]) |
| | | | ═O)═O)═O)C(O) |
| | | | ═O)═O |
| 76 | 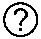 | C18OHgEPEG12, HOC18gEPEG12 | OC(CCCCCCCCC CCCCCCCC(N[C |
| | C50H93N2O19R | | @@H](CCC(NCC |
| | | | OCCOCCOCCOC |
| | | | COCCOCCOCCO |
| | | | CCOCCOCCOCC |
| | | | OCCC([R])═O)═O) |
| | | | C(O)═O)═O)═O |
| 77 | 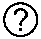 | C18OHgEPEG2PEG2, HOC18gEPEG2PEG2 | OC(CCCCCCCCC CCCCCCCC(N[C |
| | C35H62N3O12R | PEG2PEG2gEC18OH | @@H](CCC(NCC |
| | | | OCCOCC(NCCOC |
| | | | COCC([R])═O)═O) |
| | | | ═O)C(O)═O)═O)═O |
| | | | OC(CCCCCCCCC |
| | | | CCCCCCCC(N[C |
| | | | @@H(CCC(NCC |
| | | | OCCOCC(NCCOC |
| | | | COCC([R])═O)═O) |
| | | | ═O)C(O)═O)═O)═O |
| | | | OC(CCCCCCCCC |
| | | | CCCCCCCC(N[C |
| | | | @@H](CCC(NCC |
| | | | OCCOCC(NCCOC |
| | | | COCC═O)═O)═O) |
| | | | C(O)═O)═O)═O |
| 78 | 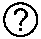 | C18OHgEPEG2PEG2S P6, | C[N+](C)(CCNC(C OCCOCCNC(COC |
| | C41H75N5O13R+ | HOC18gEPEG2PEG2S | COCCNC(CC[C@ |
| | | P6 | @H](C(O)═O)NC |
| | | | (CCCCCCCCCCCC |
| | | | CCCCC(O)═O)═O) |
| | | | ═O)═O)═O)CC([R]) |
| | | | ═O |
| 79 | 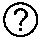 | C18OHgEPEG2SP6PE G2, | C[N+](C)(CCNC(C OCCOCCNC(CC |
| | C41H75N5O13R+ | HOC18gEPEG2SP6PE | [C@@H](C(O)═O) |
| | | G2 | NC(CCCCCCCCC |
| | | | CCCCCCCC(O)═O) |
| | | | ═O)═O)═O)CC(N |
| | | | CCOCCOCC([R]) |
| | | | ═O)═O |
| 80 | 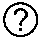 | C18OHgEPEG6, HOC18gEPEG6 | OC(CCCCCCCCC CCCCCCCC(N[C |
| | C38H69N2O13R | | @@H](CCC(NCC |
| | | | OCCOCCOCCOC |
| | | | COCCOCCC([R]) |
| | | | ═O)═O)C(O)═O) |
| | | | ═O)═O |
| 81 | 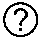 | C20OHgEPEG2PEG2, HOC20gEPEG2PEG2 | OC(CCCCCCCCC CCCCCCCCCC(N |
| | C37H66N3O12R | | [C@@H](CCC(NC |
| | | | COCCOCC(NCCO |
| | | | CCOCC([R])═O) |
| | | | ═O)═O)C(O)═O) |
| | | | ═O)═O |
| 82 | 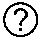 | C20gEPEG2PEG2 | CCCCCCCCCCCC CCCCCCCC(N[C |
| | C37H68N3O10R | | @@H](CCC(NCC |
| | | | OCCOCC(NCCOC |
| | | | COCC([R])═O)═O) |
| | | | ═O)C(O)═O)═O |
| 83 | 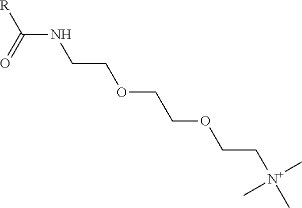 | CO(NHPEG3a) CON(PEG3a) CONHPEG3a | C[N+](C)(C)CCOC COCCNC([R])═O |
| | C10H22N2O3R+ | | |
| 84 | 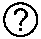 | CO(PEG12gEC18OH) | OC(CCCCCCCCC CCCCCCCC(N[C |
| | C50H94N3O19R | | @@H](CCC(NCC |
| | | | OCCOCCOCCOC |
| | | | COCCOCCOCCO |
| | | | CCOCCOCCOCC |
| | | | OCCNC([R])═O)═O) |
| | | | C(O)═O)═O)═O |
| 85 | 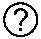 | CO(PEG2PEG2gEC18 OH) | OC(CCCCCCCCC CCCCCCCC(N[C |
| | C36H65N4O12R | | @@H](CCC(NCC |
| | | | OCCOCC(NCCOC |
| | | | COCCNC([R])═O) |
| | | | ═O)═O)C(O)═O) |
| | | | ═O)═O |
| 86 | 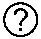 | CO(mPEG8) | COCCOCCOCCO CCOCCOCCOCC |
| | C17H36NO8R | | OCCN[R] |
| 87 | 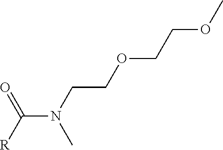 | CON(MePEG2) | CN(CCOCCOC)C ([R])═O |
| | C7H14NO3R | | |
| 88 | 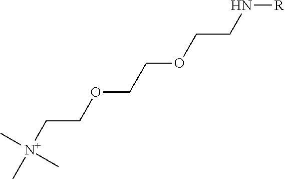 | CONH(PEG3a) | C[N+](C)(C)CCOC COCCN[R] |
| | C9H22N2O2R+ | | |
| 89 | 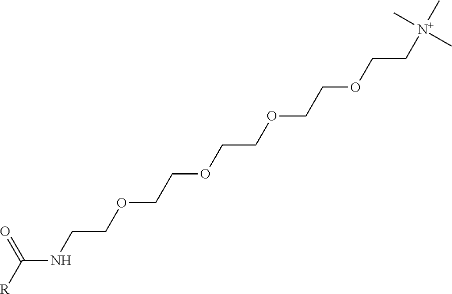 | CONH(PEG5a) | C[N+](C)(C)CCOC COCCOCCOCCN C([R])═O |
| | C14H30N2O5R+ | | |
| 90 | 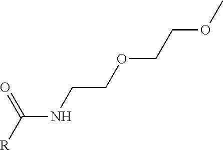 | CONH(mPEG2) | COCCOCCNC([R]) ═O |
| | C6H12NO3R | | |
| 91 | 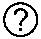 | PEG2PEG2gEC16OH | OC(CCCCCCCCC CCCCCC(N[C@@ |
| | C33H58N3O12R | | H](CCC(NCCOCC |
| | | | OCC(NCCOCCOC |
| | | | C([R])═O)═O)═O) |
| | | | C(O)═O)═O)═O |
| | | | OC(CCCCCCCCC |
| | | | CCCCCC(N[C@@ |
| | | | H](CCC(NCCOCC |
| | | | OCC(NCCOCCOC |
| | | | C═O)═O)═O)C |
| | | | (O)═O)═O)═O |
| 92 | 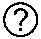 | DOTA(dPEG2) | OC(CN1CCN(CC (O)═O)CCN(CC(O) |
| | C23H40N5O10R | | ═O)CCN(CC(NCC |
| | | | OCCOCCC([R])═O) |
| | | | ═O)CC1)═O |
| 93 | 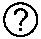 | DOTA(dPEG3) | OC(CN1CCN(CC (O)═O)CCN(CC(O) |
| | C25H44N5O11R | | ═O)CCN(CC(NCC |
| | | | OCCOCCOCCC |
| | | | ([R])═O)═O) |
| | | | CC1)═O |
| 94 | 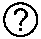 | Dab(NMeCOmPEG6) | CN(CC[C@@H](C ([R])═O)N[R])C(C |
| | C19H36N2O8R2 | | COCCOCCOCCO |
| | | | CCOCCOC)═O |
| 95 | 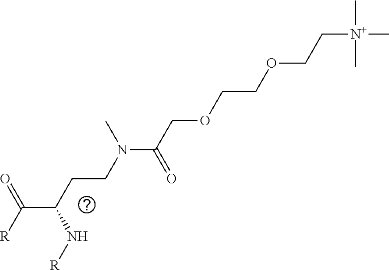 | Dab(NMecPEG2aCO), Dab(NMecPEG2a) | CN(CC[C@@H](C ([R])═O)N[R])C(C OCCOCC[N+](C) (C)C)═O |
| | C14H28N3O4R2+ | | |
| 96 | 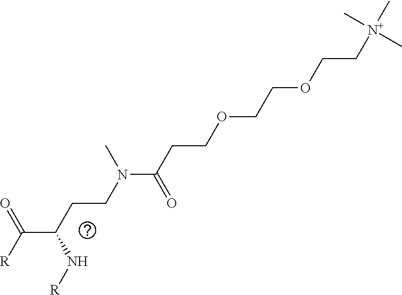 | Dab(NMecPEG3aCO), Dab(NMecPEG3a) | CN(CC[C@@H](C ([R])═O)N[R])C(C COCCOCC[N+](C) (C)C)═O |
| | C15H30N3O4R2+ | | |
| 97 | 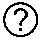 | Dab(NMecPEG5aCO), Dab(NMecPEG5a) | CN(CC[C@@H](C ([R])═O)N[R])C(C |
| | C19H38N3O6R2+ | | COCCOCCOCCO |
| | | | CC[N+](C)(C)C)═O |
| 98 | 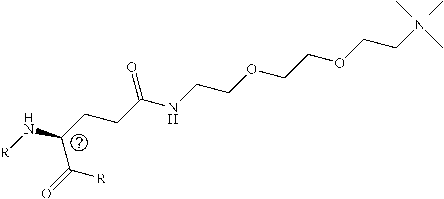 | E(COcPEG3a)) | C[N+](C)(C)CCOC COCCNC(CC[C@ @H](C([R])═O)N [R])═O |
| | C14H28N3O4R2+ | | |
| 99 | 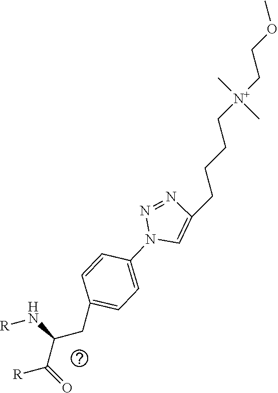 | F(4TzlDMA4mPEG) | C[N+](C)(CCCCc1 cn(-c2ccc (C[C@@H](C ([R])═O)N[R])cc2) nn1)CCOC |
| | C20H30N5O2R2+ | | |
| 100 | 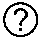 | FITCPEG4CO | Oc1cc(Oc2c(C3(c(c c4)c5cc4NC(NCC |
| | C32H33N2O10SR | | OCCOCCOCCOC |
| | | | CC([R])═O)═S)OC |
| | | | 5═O)ccc(O)c2)c3cc1 |
| 101 | 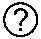 | FlagTag(dPEG2) | NCCCC[C@@H] (C(NCCOCCOCCC |
| | C48H72N11O22R | | ([R])═O)═O)NC([C |
| | | | @H](CC(O)═O)NC |
| | | | ([C@H](CC(O)═O) |
| | | | NC([C@H](CC(O) |
| | | | ═O)NC([C@H](CC |
| | | | (O)═O)NC([C@H] |
| | | | (CCCCN)NC([C@H |
| | | | (Cc(cc1)ccc1O)N |
| | | | C([C@H](CC(O)═O) |
| | | | N)═O)═O)═O)═O) |
| | | | ═O)═O)═O |
| 102 | 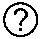 | FlagTag(dPEG3) | NCCCC[C@@H] (C(NCCOCCOCCO |
| | C50H76N11O23R | | CCC([R])═O)═O)N |
| | | | C([C@H](CC(O)═ |
| | | | O)NC([C@H](CC |
| | | | (O)═O)NC([C@H] |
| | | | (CC(O)═O)NC([C@ |
| | | | H](CC(O)═O)NC |
| | | | ([C@H](CCCCN)NC |
| | | | ([C@H](Cc(cc1)cc |
| | | | c1O)NC([C@H](C |
| | | | C(O)═O)N)═O)═O) |
| | | | ═O)═O)═O)═O)═O |
| 103 | 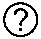 | HOC10gEPEG2PEG2, HOC10gEPEG2 | OC(CCCCCCCCC (N[C@@H](CCC(N |
| | C27H46N3O12R | PEG2CO | CCOCCOCC(NCC |
| | | | OCCOCC([R])═O) |
| | | | ═O)═O)C(O)═O) |
| | | | ═O)═O |
| | | | OC(CCCCCCCCC |
| | | | (N[C@@H](CCC(N |
| | | | CCOCCOCC(NCC |
| | | | OCCOCC═O)═O)═ |
| | | | O)C(O)═O)═O)═O |
| 104 | 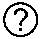 | HOC16gEPEG2PEG20 rn, | OC(CCCCCCCCC CCCCCC(N[C@@ |
| | C38H67N5O13R2 | HOC16OHgEPEG2PE | H](CCC(NCCOCC |
| | | G2orn(2) | OCC(NCCOCCOC |
| | | | C(N[C@H](CCCN |
| | | | [R])C([R])═O)═O) |
| | | | ═O)═O)C(O)═O) |
| | | | ═O)═O |
| | | | NCCC[C@H](C═O) |
| | | | NC(COCCOCCN |
| | | | C(COCCOCCNC |
| | | | (CC[C@@H](C(O) |
| | | | ═O)NC(CCCCCCC |
| | | | CCCCCCCC(O)═O) |
| | | | ═O)═O)═O)═O |
| 105 | 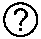 | K(BiotinPEG4) | O═C(CCCC[C@@ H]([C@H]1N2)SC |
| | C27H47N5O8SR2 | | [C@@H]1NC2═O) |
| | | | NCCOCCOCCOC |
| | | | COCCC(NCCCC |
| | | | [C@@H](C([R])═O) |
| | | | N[R])═O |
| 106 | 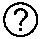 | K(FITCPEG4) | Oc1cc(Oc2c(C3(c(c c4)c5cc4NC(NCC |
| | C38H44N4O11SR2 | | OCCOCCOCCOC |
| | | | CC(NCCCC[C@@ |
| | | | H](C([R])═O)N[R]) |
| | | | ═O)═S)OC5═O)ccc |
| | | | (O)c2)c3cc1 |
| 107 | 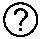 | K(NMeCOPEG4N + Me3) | CN(CCCC[C@@H] (C([R])═O)N[R])C |
| | C21H42N3O6R2+ | | (CCOCCOCCOCC |
| | | | OCC[N+](C)(C) |
| | | | C)═O |
| 108 | 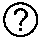 | K(NMeCOmPEG6) | CN(CCCC[C@@H] (C([R])═O)N[R])C |
| | C21H40N2O8R2 | | (CCOCCOCCOCC |
| | | | OCCOCCOC)═O |
| 109 | 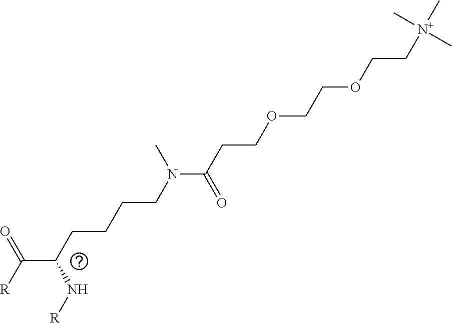 | K(NMePEG3a), K(NMecPEG3a), K(NMecPEG3aCO) | CN(CCCC[C@@H] (C([R])═O)N[R])C (CCOCCOCC[N+] (C)(C)C)═O |
| | C17H34N3O4R2+ | | |
| 110 | 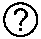 | K(NmPEG6Ac) | CC(N(CCCC[C@ @H](C([R])═O)N |
| | C21H40N2O8R2 | | [R])CCOCCOCCO |
| | | | CCOCCOCCOC)═O |
| 111 | 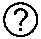 | K(PEG12NMegENMe C18OH) | CN(CCOCCOCCO CCOCCOCCOCC |
| | C58H108N4O20R2 | | OCCOCCOCCOC |
| | | | COCCOCCC(NCC |
| | | | CC[C@@H](C([R]) |
| | | | ═O)N[R])═O)C(C |
| | | | C[C@@H](C(O)═ |
| | | | O)N(C)C(CCCCC |
| | | | CCCCCCCCCCCC |
| | | | (O)═O)═O)═O |
| 112 | 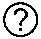 | K(PEG12NMegENMe C18Tetrazole) | CN(CCOCCOCCO CCOCCOCCOCC |
| | C59H110N8O18R2 | | OCCOCCOCCOC |
| | | | COCCOCCC(NCC |
| | | | CC[C@@H](C([R]) |
| | | | ═O)N[R])═O)C(C |
| | | | C[C@@H](C(O)═ |
| | | | O)N(C)C(CCCCC |
| | | | CCCCCCCCCCCC |
| | | | c1nnn[nH]1)═O)═O |
| 113 | 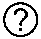 | K(PEG12gEC18OH) | OC(CCCCCCCCC CCCCCCCC(N[C |
| | C56H104N4O20R2 | | @@H](CCC(NCC |
| | | | OCCOCCOCCOC |
| | | | COCCOCCOCCO |
| | | | CCOCCOCCOCC |
| | | | OCCC(NCCCC[C |
| | | | @@H](C([R])═O) |
| | | | N[R])═O)═O)C(O) |
| | | | ═O)═O)═O |
| 114 | 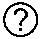 | K(PEG12gEC20OH) | OC(CCCCCCCCC CCCCCCCCCC(N |
| | C58H108N4O20R2 | | [C@@H](CCC(NC |
| | | | COCCOCCOCCO |
| | | | CCOCCOCCOCC |
| | | | OCCOCCOCCOC |
| | | | COCCC(NCCCC |
| | | | [C@@H](C([R])═O) |
| | | | N[R])═O)═O)C(O) |
| | | | ═O)═O)═O |
| 115 | 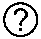 | K(PEG24C18OH) | OC(CCCCCCCCC CCCCCCCC(NCC |
| | C75H145N3O29R2 | | OCCOCCOCCOC |
| | | | COCCOCCOCCO |
| | | | CCOCCOCCOCC |
| | | | OCCOCCOCCOC |
| | | | COCCOCCOCCO |
| | | | CCOCCOCCOCC |
| | | | OCCOCCC(NCCC |
| | | | C[C@@H](C([R]) |
| | | | ═O)N[R])═O) |
| | | | ═O)═O |
| 116 | 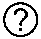 | K(PEG24gEC16OH) | OC(CCCCCCCCC CCCCCC(N[C@@ |
| | C78H148N4O32R2 | | H](CCC(NCCOCC |
| | | | OCCOCCOCCOC |
| | | | COCCOCCOCCO |
| | | | CCOCCOCCOCC |
| | | | OCCOCCOCCOC |
| | | | COCCOCCOCCO |
| | | | CCOCCOCCOCC |
| | | | OCCC(NCCCC[C |
| | | | @@H](C([R])═O) |
| | | | N[R])═O)═O)C(O) |
| | | | ═O)═O)═O |
| 117 | 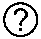 | K(PEG24gEC18OH) | C[C@](CCCCNC (CCOCCOCCOCC |
| | C81H154N4O32R2 | | OCCOCCOCCOC |
| | | | COCCOCCOCCO |
| | | | CCOCCOCCOCC |
| | | | OCCOCCOCCOC |
| | | | COCCOCCOCCO |
| | | | CCOCCOCCNC(C |
| | | | C[C@@H](C(O)═ |
| | | | O)NC(CCCCCCC |
| | | | CCCCCCCCCC(O) |
| | | | ═O)═O)═O)═O)(C |
| | | | ([R])═O)N[R] |
| 118 | 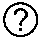 | K(PEG2NMePEG2NM egENMeC18OH) | CN(CCOCCOCC(N (C)CCOCCOCC |
| | C44H79N5O13R2 | | (NCCCC[C@@H] |
| | | | (C([R])═O)N[R])═O) |
| | | | ═O)C(CC[C@@H] |
| | | | (C(O)═O)N(C)C(C |
| | | | CCCCCCCCCCCC |
| | | | CCCC(O)═O)═O) |
| | | | ═O |
| 119 | 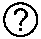 | K(PEG2NMePEG2NM egENMeC18Tetrazole) | CN(CCOCCOCC(N (C)CCOCCOCC |
| | C45H81N9O11R2 | | (NCCCC[C@@H](C |
| | | | ([R])═O)N[R])═O) |
| | | | ═O)C(CC[C@@H] |
| | | | (C(O)═O)N(C)C(C |
| | | | CCCCCCCCCCCC |
| | | | CCCCc1nnn[nH]1) |
| | | | ═O)═O |
| 120 | 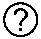 | K(PEG2PEG2Biotin) | O═C(CCCC[C@@ H]([C@H]1N2)SC |
| | C28H48N6O9SR2 | | [C@@H]1NC2═O) |
| | | | NCCOCCOCC(NC |
| | | | COCCOCC(NCCC |
| | | | C[C@@H](C([R]) |
| | | | ═O)N[R])═O)═O |
| 121 | 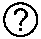 | K(PEG2PEG2C16OH) | OC(CCCCCCCCC CCCCCC(NCCOC |
| | C34H62N4O10R2 | | COCC(NCCOCCO |
| | | | CC(NCCCC[C@@ |
| | | | H](C([R])═O)N[R]) |
| | | | ═O)═O)═O)═O |
| 122 | 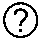 | K(PEG2PEG2C18OH) | OC(CCCCCCCCC CCCCCCCC(NCC |
| | C36H66N4O10R2 | | OCCOCC(NCCOC |
| | | | COCC(NCCCC[C |
| | | | @@H](C([R])═O) |
| | | | N[R])═O)═O)═O) |
| | | | ═O |
| 123 | 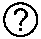 | K(PEG2PEG2DgEC18 OH) | OC(CCCCCCCCC CCCCCCCC(N[C |
| | C41H73N5O13R2 | | @H](CCC(NCCOC |
| | | | COCC(NCCOCCO |
| | | | CC(NCCCC[C@@ |
| | | | H](C([R])═O)N[R]) |
| | | | ═O)═O)═O)C(O)═ |
| | | | O)═O)═O |
| 124 | 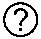 | K(PEG2PEG2PC18OH) | OC(CCCCCCCCC CCCCCCCC(N(CC |
| | C41H73N5O11R2 | | C1)[C@@H]1C(N |
| | | | CCOCCOCC(NCC |
| | | | OCCOCC(NCCCC |
| | | | [C@@H](C([R])═O) |
| | | | N[R])═O)═O)═O) |
| | | | ═O)═O |
| 125 | 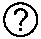 | K(PEG2PEG2PPPC18 OH) | OC(CCCCCCCCC CCCCCCCC(N(CC |
| | C51H87N7O13R2 | | C1)[C@@H]1C(N |
| | | | (CCC1)[C@@H]1C |
| | | | (N(CCC1)[C@@H] |
| | | | 1C(NCCOCCOCC |
| | | | (NCCOCCOCC(N |
| | | | CCCC[C@@H](C |
| | | | ([R])═O)N[R])═O) |
| | | | ═O)═O)═O)═O) |
| | | | ═O)═O |
| 126 | 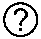 | K(PEG2PEG2PPPgEC 18OH) | OC(CCCCCCCCC CCCCCCCC(N[C |
| | C56H94N8O16R2 | | @@H](CCC(N(CC |
| | | | C1)[C@@H]1C(N |
| | | | (CCC1)[C@@H]1C |
| | | | (N(CCC1)[C@@H] |
| | | | 1C(NCCOCCOCC |
| | | | (NCCOCCOCC(N |
| | | | CCCC[C@@H](C |
| | | | ([R])═O)N[R])═O)═ |
| | | | O)═O)═O)═O)═O) |
| | | | C(O)═O)═O)═O |
| 127 | 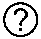 | K(PEG2PEG2PgEC18 OH) | OC(CCCCCCCCC CCCCCCCC(N[C |
| | C46H80N6O14R2 | | @@H](CCC(N(CC |
| | | | C1)[C@@H]1C(N |
| | | | CCOCCOCC(NCC |
| | | | OCCOCC(NCCCC |
| | | | [C@@H](C([R])═ |
| | | | O)N[R])═O)═O) |
| | | | ═O)═O)C(O)═O) |
| | | | ═O)═O |
| 128 | 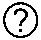 | K(PEG2PEG2Sp6g EC18OH) | C[N+](C)(CCNC(C C[C@@H](C(O)═ |
| | C47H86N7O14R2+ | | O)NC(CCCCCCC |
| | | | CCCCCCCCCC(O) |
| | | | ═O)═O)═O)CC(NC |
| | | | COCCOCC(NCCO |
| | | | CCOCC(NCCCC |
| | | | [C@@H](C([R])═O) |
| | | | N[R])═O)═O)═O |
| 129 | 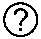 | K(PEG2PEG2Trxg EC18OH) | OC(CCCCCCCCC CCCCCCCC(N[C |
| | C49H86N6O14R2 | | @@H](CCC(NC[C |
| | | | @H](CC1)CC[C@ |
| | | | @H]1C(NCCOCC |
| | | | OCC(NCCOCCOC |
| | | | C(NCCCC[C@@H] |
| | | | (C([R])═O)N[R])═ |
| | | | O)═O)═O)═O)C(O) |
| | | | ═O)═O)═O |
| 130 | 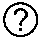 | K(PEG2PEG2Trxg EC20OH) | OC(CCCCCCCCC CCCCCCCCCC(N |
| | C51H90N6O14R2 | | [C@@H](CCC(NC |
| | | | [C@H](CC1)CC[C |
| | | | @@H]1C(NCCOC |
| | | | COCC(NCCOCCO |
| | | | CC(NCCCC[C@@ |
| | | | H](C([R])═O)N[R]) |
| | | | ═O)═O)═O)═O)C |
| | | | (O)═O)═O)═O |
| 131 | 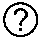 | K(PEG2PEG2Trxg ETrxC20OH) | OC(CCCCCCCCC CCCCCCCCCC(N |
| | C59H103N7O15R2 | | C[C@H](CC1)CC |
| | | | [C@@H]1C(N[C@ |
| | | | @H](CCC(NC[C@ |
| | | | H](CC1)CC[C@H] |
| | | | 1C(NCCOCCOCC |
| | | | (NCCOCCOCC(NC |
| | | | CCC[C@@H](C |
| | | | ([R])═O)N[R])═O)═ |
| | | | O)═O)═O)C(O)═O) |
| | | | ═O)═O)═O |
| 132 | 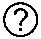 | K(PEG2PEG2g EC10OH) | OC(CCCCCCCCC (N[C@@H](CCC(N |
| | C33H57N5O13R2 | | CCOCCOCC(NCC |
| | | | OCCOCC(NCCCC |
| | | | [C@@H](C([R])═O) |
| | | | N[R])═O)═O)═O) |
| | | | C(O)═O)═O)═O |
| 133 | 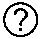 | K(PEG2PEG2gEC12) | CCCCCCCCCCCC (N[C@@H](CCC |
| | C35H63N5O11R2 | | (NCCOCCOCC(NC |
| | | | COCCOCC(NCCCC |
| | | | [C@@H](C([R])═O) |
| | | | N[R])═O)═O)═O) |
| | | | C(O)═O)═O |
| 134 | 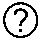 | K(PEG2PEG2gEC14) NMeK(PEG2PEG2g | CCCCCCCCCCCC CC(N[C@@H](CC |
| | C37H67N5O11R2 | EC14) | C(NCCOCCOCC |
| | | | (NCCOCCOCC(NC |
| | | | CCC[C@@H](C([R]) |
| | | | ═O)N[R])═O)═O) |
| | | | ═O)C(O)═O)═O |
| 135 | 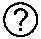 | K(PEG2PEG2gEC16) | CCCCCCCCCCCC CCCC(N[C@@H] |
| | C39H71N5O11R2 | | (CCC(NCCOCCOC |
| | | | C(NCCOCCOCC |
| | | | (NCCCC[C@@H] |
| | | | (C([R])═O)N[R])═O) |
| | | | ═O)═O)C(O)═O)═O |
| 136 | 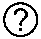 | K(PEG2PEG2gEC16 OH) | OC(CCCCCCCCC CCCCCC(N[C@@ |
| | C39H69N5O13R2 | | H](CCC(NCCOCC |
| | | | OCC(NCCOCCOC |
| | | | C(NCCCC[C@@H] |
| | | | (C([R])═O)N[R])═O) |
| | | | ═O)═O)C(O)═O) |
| | | | ═O)═O |
| 137 | 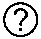 | K(PEG2PEG2gEC16 tetrazole) | OC([C@H](CCC(N CCOCCOCC(NCC |
| | C40H71N9O11R2 | | OCCOCC(NCCCC |
| | | | [C@@H](C([R])═O) |
| | | | N[R])═O)═O)═O) |
| | | | NC(CCCCCCCCC |
| | | | CCCCCCc1nnn[nH] |
| | | | 1)═O)═O |
| 138 | 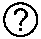 | K(PEG2PEG2gEC18) | CCCCCCCCCCCC CCCCCC(N[C@@H] |
| | C41H75N5O11R2 | | (CCC(NCCOCC |
| | | | OCC(NCCOCCOC |
| | | | C(NCCCC[C@@H] |
| | | | (C([R])═O)N[R])═O) |
| | | | ═O)═O)C(O)═O)═O |
| 139 | 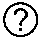 | K(PEG2PEG2gEC18 tetrazole) | OC([C@H](CCC(N CCOCCOCC(NCC |
| | C42H75N9O11R2 | | OCCOCC(NCCCC |
| | | | [C@@H](C([R])═O) |
| | | | N[R])═O)═O)═O) |
| | | | NC(CCCCCCCCC |
| | | | CCCCCCCCc1nnn |
| | | | [nH]1)═O)═O |
| 140 | 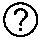 | K(PEG2PEG2gEC20 OH) | OC(CCCCCCCCC CCCCCCCCCC(N |
| | C43H77N5O13R2 | | [C@@H](CCC(NC |
| | | | COCCOCC(NCCO |
| | | | CCOCC(NCCCC |
| | | | [C@@H](C([R])═O) |
| | | | N[R])═O)═O)═O) |
| | | | C(O)═O)═O)═O |
| 141 | 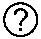 | KPEG2PEG2gEDap (C16OH)2, | OC(CCCCCCCCC CCCCCC(NC[C@ |
| | C58H103N7O17R2 | K(PEG2PEG2gEDAP | @H](C(N[C@@H] |
| | | (C16OH)2) | (CCC(NCCOCCO |
| | | | CC(NCCOCCOCC |
| | | | (NCCCC[C@@H] |
| | | | (C([R])═O)N[R])═O) |
| | | | ═O)═O)C(O)═O)═O) |
| | | | NC(CCCCCCC |
| | | | CCCCCCCC(O)═O) |
| | | | ═O)═O)═O |
| 142 | 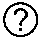 | K(PEG2PEG2gEDAP (mXOH)2) | OC([C@H](CCC(N CCOCCOCC(NCC |
| | C60H91N7O19R2 | KPEG2PEG2gEDAP | OCCOCC(NCCCC |
| | | (mXOH)2 | [C@@H](C([R])═ |
| | | | O)N[R])═O)═O)═O) |
| | | | NC([C@H](CNC |
| | | | (CCCCCCCCCOc1 |
| | | | cc(C(O)═O)ccc1)═ |
| | | | O)NC(CCCCCCC |
| | | | CCOc1cc(C(O)═O) |
| | | | ccc1)═O)═O)═O |
| 143 | 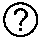 | K(PEG2PEG2gEDAP (pXOH)2) | OC([C@H](CCC(N CCOCCOCC(NCC |
| | C60H91N7O19R2 | KPEG2PEG2gEDAP | OCCOCC(NCCCC |
| | | (pXOH)2 | [C@@H](C([R])═ |
| | | | O)N[R])═O)═O)═O) |
| | | | NC([C@H](CNC |
| | | | (CCCCCCCCCOc(c |
| | | | c1)ccc1C(O)═O)═O) |
| | | | NC(CCCCCCCCC |
| | | | Oc(cc1)ccc1C(O)═ |
| | | | O)═O)═O)═O |
| 144 | 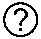 | K(PEG2PEG2gESp6 C18OH) | C[N+](C)(CCNC(C CCCCCCCCCCCC |
| | C47H86N7O14R2+ | | CCCC(O)═O)═O)C |
| | | | C(N[C@@H](CCC |
| | | | (NCCOCCOCC(N |
| | | | CCOCCOCC(NCC |
| | | | CC[C@@H](C([R]) |
| | | | ═O)N[R])═O)═O) |
| | | | ═O)C(O)═O)═O |
| 145 | 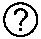 | K(PEG2PEG2gETrx C18OH) | OC(CCCCCCCCC CCCCCCCC(NC[C |
| | C49H86N6O14R2 | | @H](CC1)CC[C@ |
| | | | @H]1C(N[C@@H] |
| | | | (CCC(NCCOCCO |
| | | | CC(NCCOCCOCC |
| | | | (NCCCC[C@@H] |
| | | | (C([R])═O)N[R])═O) |
| | | | ═O)═O)C(O)═O)═ |
| | | | O)═O)═O |
| 146 | 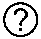 | K(PEG2PEG2gETrx C20OH) | OC(CCCCCCCCC CCCCCCCCCC(N |
| | C51H90N6O14R2 | | C[C@H](CC1)CC |
| | | | [C@@H]1C(N[C@ |
| | | | @H](CCC(NCCOC |
| | | | COCC(NCCOCCO |
| | | | CC(NCCCC[C@@ |
| | | | H](C([R])═O)N[R]) |
| | | | ═O)═O)═O)C(O)═ |
| | | | O)═O)═O)═O |
| 147 | 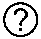 | K(PEG2PEG2gEmX OH) | OC([C@H](CCC(N CCOCCOCC(NCC |
| | C40H63N5O14R2 | | OCCOCC(NCCCC |
| | | | [C@@H](C([R])═O) |
| | | | N[R])═O)═O)═O) |
| | | | NC(CCCCCCCCC |
| | | | Oc1cc(C(O)═O)ccc1) |
| | | | ═O)═O |
| 148 | 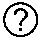 | K(PEG2PEG2gEpXOH) | OC([C@H](CCC(N CCOCCOCC(NCC |
| | C40H63N5O14R2 | | OCCOCC(NCCCC |
| | | | [C@@H](C([R])═O) |
| | | | N[R])═O)═O)═O) |
| | | | NC(CCCCCCCCC |
| | | | Oc(cc1)ccc1C(O) |
| | | | ═O)═O)═O |
| 149 | 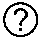 | K(PEG2PEG2pC18OH) | OC(CCCCCCCCC CCCCCCCC(N(CC |
| | C41H73N5O11R2 | | C1)[C@H]1C(NCC |
| | | | OCCOCC(NCCOC |
| | | | COCC(NCCCC[C |
| | | | @@H](C([R])═O) |
| | | | N[R])═O)═O)═O) |
| | | | ═O)═O |
| 150 | 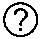 | K(PEG2PEG2pg EC18OH) | OC(CCCCCCCCC CCCCCCCC(N[C |
| | C46H80N6O14R2 | | @@H](CCC(N(CC |
| | | | C1)[C@H]1C(NCC |
| | | | OCCOCC(NCCOC |
| | | | COCC(NCCCC[C |
| | | | @@H](C([R])═O) |
| | | | N[R])═O)═O)═O)═ |
| | | | O)C(O)═O)═O)═O |
| 151 | 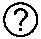 | K(PEG2PEG2ppp C18OH) | OC(CCCCCCCCC CCCCCCCC(N(CC |
| | C51H87N7O13R2 | | C1)[C@H]1C(N(C |
| | | | CC1)[C@H]1C(N |
| | | | (CCC1)[C@H]1C(N |
| | | | CCOCCOCC(NCC |
| | | | OCCOCC(NCCCC |
| | | | [C@@H](C([R])═O) |
| | | | N[R])═O)═O)═O) |
| | | | ═O)═O)═O)═O |
| 152 | 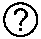 | K(PEG2PEG2pppg EC18OH) | OC(CCCCCCCCC CCCCCCCC(N[C |
| | C56H94N8O16R2 | | @@H](CCC(N(CC |
| | | | C1)[C@H]1C(N(C |
| | | | CC1)[C@H]1C(N |
| | | | (CCC1)[C@H]1C(N |
| | | | CCOCCOCC(NCC |
| | | | OCCOCC(NCCCC |
| | | | [C@@H](C([R])═ |
| | | | O)N[R])═O)═O)═O) |
| | | | ═O)═O)═O)C(O)═ |
| | | | O)═O)═O |
| 153 | 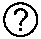 | K(PEG2PEG6g EC16OH) | OC(CCCCCCCCC CCCCCC(N[C@@ |
| | C48H87N5O17R2 | | H](CCC(NCCOCC |
| | | | OCCOCCOCCOC |
| | | | COCCC(NCCOCC |
| | | | OCC(NCCCC[C@ |
| | | | @H](C([R])═O)N |
| | | | [R])═O)═O)═O)C(O) |
| | | | ═O)═O)═O |
| 154 | 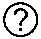 | K(PEG2PEG6g EC18OH) | OC(CCCCCCCCC CCCCCCCC(N[C |
| | C50H91N5O17R2 | | @@H](CCC(NCC |
| | | | OCCOCCOCCOC |
| | | | COCCOCCC(NCC |
| | | | OCCOCC(NCCCC |
| | | | [C@@H](C([R])═O) |
| | | | N[R])═O)═O)═O) |
| | | | C(O)═O)═O)═O |
| 155 | 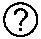 | K(PEG2Sp6PEG2g EC18OH) | C[N+](C)(CCNC(C OCCOCCNC(CC |
| | C47H86N7O14R2+ | | [C@@H](C(O)═O) |
| | | | NC(CCCCCCCCC |
| | | | CCCCCCCC(O)═O) |
| | | | ═O)═O)═O)CC(N |
| | | | CCOCCOCC(NCC |
| | | | CC[C@@H](C([R]) |
| | | | ═O)N[R])═O)═O |
| 156 | 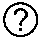 | K(PEG2gEC16OH) | OC(CCCCCCCCC CCCCCC(N[C@@ |
| | C33H58N4O10R2 | | H](CCC(NCCOCC |
| | | | OCC(NCCCC[C@ |
| | | | @H](C([R])═O)N |
| | | | [R])═O)═O)C(O)═O) |
| | | | ═O)═O |
| 157 | 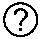 | K(PEG2gEC18OH) | OC(CCCCCCCCC CCCCCCCC(N[C |
| | C35H62N4O10R2 | | @@H](CCC(NCC |
| | | | OCCOCC(NCCCC |
| | | | [C@@H](C([R])═O) |
| | | | N[R])═O)═O)C |
| | | | (O)═O)═O)═O |
| 158 | 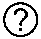 | K(PEG2gEgEPEG24S BC16Tetrazole) | OC([C@H](CCC(N CCOCCOCC(NCC |
| | C49H85N11O17SR2 | | CC[C@@H](C([R]) |
| | | | ═O)N[R])═O)═O) |
| | | | NC(CC[C@@H](C |
| | | | (O)═O)NC(COCC |
| | | | OCCNC(CCCS(NC |
| | | | (CCCCCCCCCCC |
| | | | CCCCc1nnn[nH]1) |
| | | | ═O)(═O)═O)═O) |
| | | | ═O)═O)═O |
| 159 | 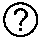 | K(PEG3OMe) K(mPEG4) | COCCOCCOCCO CCC(NCCCC[C@ |
| | C16H30N2O6R2 | | @H](C([R])═O)N |
| | | | [R])═O |
| 160 | 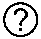 | K(PEG4Biotina), K(PEG4Biotin) | O═C(CCCC[C@H] ([C@@H]1N2)SC |
| | C27H47N5O8SR2 | | [C@H]1NC2═O)NC |
| | | | COCCOCCOCCO |
| | | | CCC(NCCCC[C@ |
| | | | @H](C([R])═O)N |
| | | | [R])═O |
| 161 | 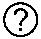 | K(PEG6Biotin) | O═C(CCCC[C@@ H]([C@H]1N2)SC |
| | C31H55N5O10SR2 | | [C@@H]1NC2═O) |
| | | | NCCOCCOCCOC |
| | | | COCCOCCOCCC |
| | | | (NCCCC[C@@H] |
| | | | (C([R])═O)N[R])═O |
| 162 | 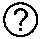 | K(PEG6PEG6g EC16OH) | OC(CCCCCCCCC CCCCCC(N[C@@ |
| | C57H105N5O21R2 | | H](CCC(NCCOCC |
| | | | OCCOCCOCCOC |
| | | | COCCC(NCCOCC |
| | | | OCCOCCOCCOC |
| | | | COCCC(NCCCC |
| | | | [C@@H](C([R])═O) |
| | | | N[R])═O)═O)═O) |
| | | | C(O)═O)═O)═O |
| 163 | 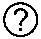 | K(PEG6PEG6g EC18OH) | OC(CCCCCCCCC CCCCCCCC(N[C |
| | C59H109N5O21R2 | | @@H](CCC(NCC |
| | | | OCCOCCOCCOC |
| | | | COCCOCCC(NCC |
| | | | OCCOCCOCCOC |
| | | | COCCOCCC(NCC |
| | | | CC[C@@H](C([R]) |
| | | | ═O)N[R])═O)═O) |
| | | | ═O)C(O)═O)═O)═O |
| 164 | 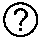 | K(PEG6gEC16OH) | OC(CCCCCCCCC CCCCCC(N[C@@ |
| | C42H76N4O14R2 | | H](CCC(NCCOCC |
| | | | OCCOCCOCCOC |
| | | | COCCC(NCCCC |
| | | | [C@@H](C([R])═O) |
| | | | N[R])═O)═O)C |
| | | | (O)═O)═O)═O |
| 165 | 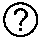 | K(PEG6gEC18OH) | OC(CCCCCCCCC CCCCCCCC(N[C |
| | C44H80N4O14R2 | | @@H](CCC(NCC |
| | | | OCCOCCOCCOC |
| | | | COCCOCCC(NCC |
| | | | CC[C@@H](C([R]) |
| | | | ═O)N[R])═O)═O) |
| | | | C(O)═O)═O)═O |
| 166 | 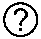 | K(Sp6PEG2PEG2g EC18OH) | C[N+](C)(CCNC(C OCCOCCNC(COC |
| | C47H86N7O14R2+ | | COCCNC(CC[C@ |
| | | | @H](C(O)═O)NC |
| | | | (CCCCCCCCCCCC |
| | | | CCCCC(O)═O)═O) |
| | | | ═O)═O)═O)CC(NC |
| | | | CCC[C@@H](C |
| | | | ([R])═O)N[R])═O |
| 167 | 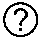 | K(cPEG3a), K(cPEG3aCO) | C[N+](C)(C)CCOC COCCC(NCCCC |
| | C16H32N3O4R2+ | | [C@@H](C([R])═O) |
| | | | N[R])═O |
| 168 | 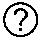 | K(dPEG12Ac) | CC(NCCOCCOCC OCCOCCOCCOC |
| | C35H67N3O15R2 | | COCCOCCOCCO |
| | | | CCOCCOCCC(NC |
| | | | CCC[C@@H](C |
| | | | ([R])═O)N[R]) |
| | | | ═O)═O |
| 169 | 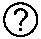 | K(dPEG12AcBr) | O═C(CCOCCOCC OCCOCCOCCOC |
| | C35H66BrN3O15R2 | | COCCOCCOCCO |
| | | | CCOCCOCCNC(C |
| | | | Br)═O)NCCCC[C |
| | | | @@H](C([R])═O) |
| | | | N[R] |
| 170 | 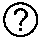 | K(dPEG6Ac) | CC(NCCOCCOCC OCCOCCOCCOC |
| | C23H43N3O9R2 | | CC(NCCCC[C@@ |
| | | | H](C([R])═O)N[R]) |
| | | | ═O)═O |
| 171 | 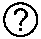 | K(dPEG6AcBr) | O═C(CCOCCOCC OCCOCCOCCOC |
| | C23H42BrN3O9R2 | | CNC(CBr)═O)NCC |
| | | | CC[C@@H](C([R]) |
| | | | ═O)N[R] |
| 172 | 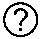 | K(dPEG9Ac) | CC(NCCOCCOCC OCCOCCOCCOC |
| | C29H55N3O12R2 | | COCCOCCOCCC |
| | | | (NCCCC[C@@H] |
| | | | (C([R])═O)N[R]) |
| | | | ═O)═O |
| 173 | 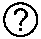 | K(dPEG9AcBr) | O═C(CCOCCOCC OCCOCCOCCOC |
| | C29H54BrN3O12R2 | | COCCOCCOCCN |
| | | | C(CBr)═O)NCCCC |
| | | | [C@@H](C |
| | | | ([R])═O)N[R] |
| 174 | 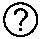 | K(mPEG12) | COCCOCCOCCO CCOCCOCCOCC |
| | C32H62N2O14R2 | | OCCOCCOCCOC |
| | | | COCCC(NCCCC |
| | | | [C@@H](C |
| | | | ([R])═O)N[R])═O |
| 175 | 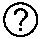 | PEG2PEG2gEC18 | CCCCCCCCCCCC CCCCCC(N[C@@ |
| | C35H64N3O10R | | H](CCC(NCCOCC |
| | | | OCC(NCCOCCOC |
| | | | C([R])═O)═O)═O) |
| | | | C(O)═O)═O |
| | | | CCCCCCCCCCCC |
| | | | CCCCCC(N[C@@ |
| | | | H](CCC(NCCOCC |
| | | | OCC(NCCOCCOC |
| | | | C═O)═O)═O)C(O) |
| | | | ═O)═O |
| 176 | 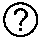 | Lys(N + Me2mPEG3) | C[N+](C)(CCCC[C @@H](C([R])═O) |
| | C13H27N2O3R2+ | | N[R])CCOCCOC |
| 177 | 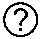 | LysQuatMe2mPEG3, Lys(N + (Me)2mPEG3) | C[N+](C)(CCCC[C @@H](C═O)N)CC |
| | C13H29N2O3+ | | OCCOC |
| 178 | 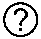 | MMPEG3F | C[N+]1(CCOCCO CCOc2ccc(C[C@ |
| | C20H31N2O5R2+ | | @H](C([R])═O)N |
| | | | [R])cc2)CCOCC1 |
| 179 | 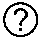 | N(PEG2PEG2gEC18 OH)Gly | OC(CCCCCCCCC CCCCCCCC(N[C |
| | C37H66N4O12R2 | | @@H](CCC(NCC |
| | | | OCCOCC(NCCOC |
| | | | COCCN(CC([R])═ |
| | | | O)[R])═O)═O)C(O) |
| | | | ═O)═O)═O |
| 180 | 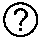 | NMeK(PEG12C18OH) | CN([C@@H](CCC CNC(CCOCCOCC |
| | C52H99N3O17R2 | | OCCOCCOCCOC |
| | | | COCCOCCOCCO |
| | | | CCOCCOCCNC(C |
| | | | CCCCCCCCCCCC |
| | | | CCCC(O)═O)═O) |
| | | | ═O)C([R])═O)[R] |
| 181 | 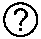 | NMeK(PEG12g EC18OH) | CN([C@@H](CCC CNC(CCOCCOCC |
| | C57H106N4O20R2 | | OCCOCCOCCOC |
| | | | COCCOCCOCCO |
| | | | CCOCCOCCNC(CC |
| | | | [C@@H](C(O)═O) |
| | | | NC(CCCCCCC |
| | | | CCCCCCCCCC(O) |
| | | | ═O)═O)═O)═O)C |
| | | | ([R])═O)[R] |
| 182 | 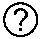 | NMeK(PEG2NMePEG 2NMegENMeC18OH) | CN(CCOCCOCC (N(C)CCOCCOCC |
| | C45H81N5O13R2 | | (NCCCC[C@@H] |
| | | | (C([R])═O)N(C)[R]) |
| | | | ═O)═O)C(CC[C@ |
| | | | @H](C(O)═O)N(C) |
| | | | C(CCCCCCCCCC |
| | | | CCCCCCC(O)═O) |
| | | | ═O)═O |
| 183 | 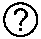 | NMeK(PEG2PEG2C12) | CCCCCCCCCCCC (NCCOCCOCC(N |
| | C31H58N4O8R2 | | CCOCCOCC(NCC |
| | | | CC[C@@H](C([R]) |
| | | | ═O)N(C)[R])═O) |
| | | | ═O)═O |
| 184 | 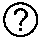 | NMeK (PEG2PEG2gEC12) | CCCCCCCCCCCC (N[C@@H](CCC |
| | C36H65N5O11R2 | | (NCCOCCOCC(NC |
| | | | COCCOCC(NCCCC |
| | | | [C@@H](C([R])═O) |
| | | | N(C)[R])═O)═O) |
| | | | ═O)C(O)═O)═O |
| 185 | 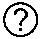 | NMeK(PEG2PEG2g EC16OH) | CN([C@@H](CCC CNC(COCCOCCN |
| | C40H71N5O13R2 | | C(COCCOCCNC |
| | | | (CC[C@@H](C(O) |
| | | | ═O)NC(CCCCCCC |
| | | | CCCCCCCC(O)═O) |
| | | | ═O)═O)═O)═O)C |
| | | | ([R])═O)[R] |
| 186 | 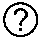 | NMeK(PEG2PEG2g EC18OH) | CN([C@@H](CCC CNC(COCCOCCN |
| | C42H75N5O13R2 | | C(COCCOCCNC |
| | | | (CC[C@@H](C(O) |
| | | | ═O)NC(CCCCCCC |
| | | | CCCCCCCCCC(O) |
| | | | ═O)═O)═O)═O) |
| | | | ═O)C([R])═O)[R] |
| 187 | 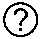 | NMeK(PEG2PEG2g EC20OH) | CN([C@@H](CCC CNC(COCCOCCN |
| | C44H79N5O13R2 | | C(COCCOCCNC |
| | | | (CC[C@@H](C(O) |
| | | | ═O)NC(CCCCCCC |
| | | | CCCCCCCCCCCC |
| | | | (O)═O)═O)═O)═O) |
| | | | ═O)C([R])═O)[R] |
| 188 | 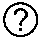 | NMeK(PEG6C18OH) | CN([R])[R](CCCC NC(CCOCCOCCO |
| | C39H74N3O11R3 | | CCOCCOCCOCC |
| | | | NC(CCCCCCCCC |
| | | | CCCCCCCC(O)═O) |
| | | | ═O)═O)C([R])═O |
| 189 | 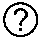 | NMeK(PEG6gEC18 OH) | CN([C@@H](CCC CNC(CCOCCOCC |
| | C45H82N4O14R2 | | OCCOCCOCCOC |
| | | | CNC(CC[C@@H] |
| | | | (C(O)═O)NC(CCCC |
| | | | CCCCCCCCCCCC |
| | | | C(O)═O)═O)═O)═ |
| | | | O)C([R])═O)[R] |
| 190 | 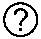 | NMeK(SP6PEG2g EC18OH) | CN([C@@H](CCC CNC(C[N+](C)(C) |
| | C42H77N6O11R2+ | | CCNC(COCCOCC |
| | | | NC(CC[C@@H](C |
| | | | (O)═O)NC(CCCCC |
| | | | CCCCCCCCCCCC |
| | | | (O)═O)═O)═O)═O) |
| | | | ═O)C([R])═O)[R] |
| 191 | 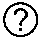 | PEG12gEC18OH | OC(CCCCCCCCC CCCCCCCC(N[C |
| | C50H94N3O19R | | @@H](CCC(NCC |
| | | | OCCOCCOCCOC |
| | | | COCCOCCOCCO |
| | | | CCOCCOCCOCC |
| | | | OCCC(N[R])═O)═ |
| | | | O)C(O)═O)═O)═O |
| 192 | 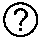 | PEG12gEC20OH | OC(CCCCCCCCC CCCCCCCCCC(N |
| | C52H98N3O19R | | [C@@H](CCC(NC |
| | | | COCCOCCOCCO |
| | | | CCOCCOCCOCC |
| | | | OCCOCCOCCOC |
| | | | COCCC(N[R])═O) |
| | | | ═O)C(O)═O)═O)═O |
| 193 | 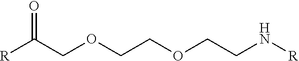 | PEG2, PEG2(2) | O═C(COCCOCCN [R])[R] |
| | C6H11NO3R2 | | |
| 194 | 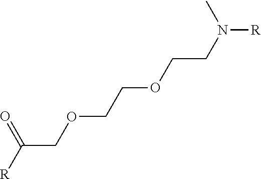 | PEG2(NMe(2)) PEG2NMe | CN(CCOCCOCC ([R])═O)[R] |
| | C7H13NO3R2 | | |
| 195 | 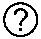 | PEG2PEG2eKC16OH | OC(CCCCCCCCC CCCCCC(N[C@@ |
| | C34H63N4O11R | | H](CCCCNC(COC |
| | | | COCCNC(COCCO |
| | | | CCN[R])═O)═O)C |
| | | | (O)═O)═O)═O |
| | | | NCCOCCOCC(NC |
| | | | COCCOCC(NCCC |
| | | | C[C@@H](C(O)═O) |
| | | | NC(CCCCCCC |
| | | | CCCCCCCC(O)═O) |
| | | | ═O)═O)═O |
| 196 | 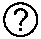 | PEG2PEG2eKC18OH | OC(CCCCCCCCC CCCCCCCC(N[C |
| | C36H67N4O11R | | @@H](CCCCNC |
| | | | (COCCOCCNC(CO |
| | | | CCOCCN[R])═O) |
| | | | ═O)C(O)═O)═O)═O |
| | | | NCCOCCOCC(NC |
| | | | COCCOCC(NCCC |
| | | | C[C@@H](C(O)═ |
| | | | O)NC(CCCCCCC |
| | | | CCCCCCCCCC(O) |
| | | | ═O)═O)═O)═O |
| 197 | 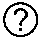 | PEG2PEG2gDabC18OH | OC(CCCCCCCCC CCCCCCCC(N[C |
| | C34H63N4O11R | | @@H](CCNC(CO |
| | | | CCOCCNC(COCC |
| | | | OCCN[R])═O)═O) |
| | | | C(O)═O)═O)═O |
| | | | NCCOCCOCC(NC |
| | | | COCCOCC(NCC |
| | | | [C@@H](C(O)═O) |
| | | | NC(CCCCCCCCC |
| | | | CCCCCCCC(O)═O) |
| | | | ═O)═O)═O |
| 198 | 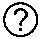 | PEG2PEG2gEC20OH | OC(CCCCCCCCC CCCCCCCCCC(N |
| | C37H67N4O12R | | [C@@H](CCC(NC |
| | | | COCCOCC(NCCO |
| | | | CCOCC(N[R])═O) |
| | | | ═O)═O)C(O)═O) |
| | | | ═O)═O |
| 199 | 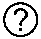 | PEG6 | O═C(CCOCCOCC OCCOCCOCCOC |
| | C15H29NO7R2 | | CN[R])[R] |
| 200 | 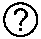 | Peg12-Ome Peg12OMe, | COCCOCCOCCO CCOCCOCCOCC |
| | C26H52O13 | Polyethylene 12-O- | OCCOCCOCCOC |
| | | Methyl | COCCC═O |
| | | Peg12-O methyl | |
| 201 | 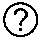 | Peg120Me, Peg12- Omethyl | CCOCCOCCOCC OCCOCCOCCOC |
| | C23H48O11 | | COCCOCCOCCO |
| | | | C |
| 202 | 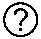 | Pip(PEG12gEC16), Spiral_Pip_PEG12_Iso | CCCCCCCCCCCC CCCC(N[C@@H] |
| | C54H100N4O18R2 | Glu_Palm | (CCC(NCCOCCOC |
| | | | COCCOCCOCCO |
| | | | CCOCCOCCOCC |
| | | | OCCOCCOCCC(N |
| | | | (CC1)CCC1(C([R]) |
| | | | ═O)N[R])═O)═O)C |
| | | | (O)═O)═O |
| 203 | 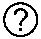 | TMAPF(PEG2PEG2g EC18OH) | C[N+](C)(CCCCC Oc1ccc(C[C@@H] |
| | C51H88N5O13R2+ | | (C([R])═O)N[R])cc |
| | | | 1)CCOCCOCCNC |
| | | | (COCCOCCNC(CC |
| | | | [C@@H](C(O)═O) |
| | | | NC(CCCCCCCCC |
| | | | CCCCCCCC(O)═O) |
| | | | ═O)═O)═O |
| 204 | 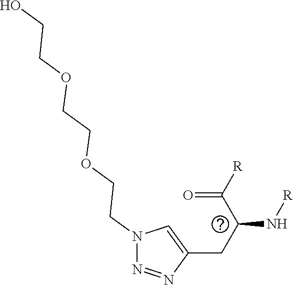 | Tzl(PEG3OH) | OCCOCCOCCn1n nc(C[C@@H](C ([R])═O)N[R])c1 |
| | C11H18N4O4R2 | | |
| 205 | 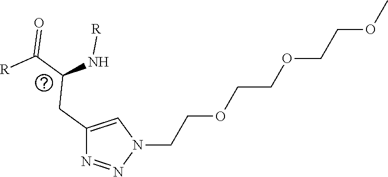 | Tzl(mPEG3) | COCCOCCOCCn1 nnc(C[C@@H](C ([R])═O)N[R])c1 |
| | C12H20N4O4R2 | | |
| 206 | 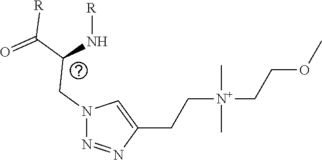 | TzlChmPEG | C[N+](C)(CCc1cn (C[C@@H](C([R]) ═O)N[R])nn1)CCOC |
| | C12H22N5O2R2+ | | |
| 207 | 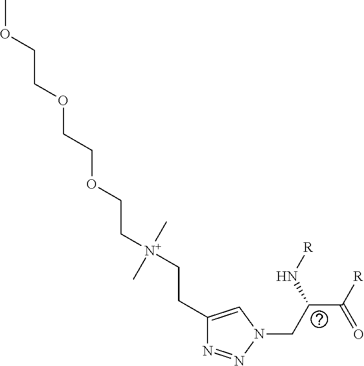 | TzlChmPEG3 | C[N+](C)(CCc1cn (C[C@@H](C([R]) ═O)N[R])nn1)CCO CCOCCOC |
| | C16H30N5O4R2+ | | |
| 208 | 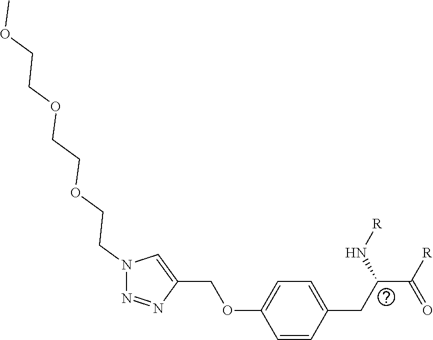 | Y(OTzl(mPEG3)) | COCCOCCOCCn1 nnc(COc2ccc(C[C @@H](C([R])═O) N[R])cc2)c1 |
| | C19H26N4O5R2 | | |
| 209 | 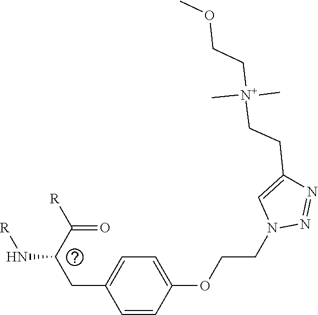 | Y(OTzlChmPEG) | C[N+](C)(CCc1cn (CCOc2ccc(C[C@ @H](C([R])═O)N [R])cc2)nn1)CCOC |
| | C20H30N5O3R2+ | | |
| 210 | 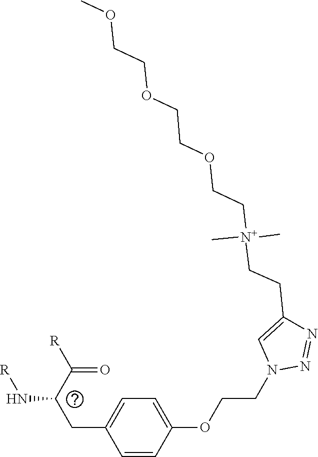 | Y(OTzlChmPEG3) | C[N+](C)(CCc1cn (CCOc2ccc(C[C@ @H](C([R])═O)N [R])cc2)nn1)CCOC COCCOC |
| | C24H38N5O5R2+ | | |
| 211 | 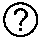 | YC8CO(NHPEG3a) | C[N+](C)(C)CCOC COCCNC(CCCCC |
| | C27H46N3O5R2+ | | CCCOc1ccc(C[C@ |
| | | | @H](C([R])═O)N |
| | | | [R])cc1)═O |
| 212 | 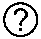 | aMeK(PEG12gEC16) | CCCCCCCCCCCC CCCC(N[C@@H] |
| | C55H104N4O18R2 | | (CCC(NCCOCCOC |
| | | | COCCOCCOCCO |
| | | | CCOCCOCCOCC |
| | | | OCCOCCOCCC(N |
| | | | CCCC[C@@](C) |
| | | | (C([R])═O)N[R])═O) |
| | | | ═O)C(O)═O)═O |
| 213 | 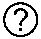 | aMeK(PEG12gEC18 OH) | C[C@@H](C═O)N C([C@](C)(CCCC |
| | C62H116N6O22 | | NC(CCOCCOCCO |
| | | | CCOCCOCCOCC |
| | | | OCCOCCOCCOC |
| | | | COCCOCCNC(CC |
| | | | [C@@H](C(O)═O) |
| | | | NC(CCCCCCCCC |
| | | | CCCCCCCC(O)═O) |
| | | | ═O)═O)═O)NC |
| | | | (CN)═O)═O |
| 214 | 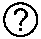 | aMeK(PEG2PEG2g EC16OH) | C[C@](CCCCNC (COCCOCCNC(CO |
| | C40H71N5O13R2 | | CCOCCNC(CC[C |
| | | | @@H](C(O)═O)N |
| | | | C(CCCCCCCCCC |
| | | | CCCCC(O)═O)═O) |
| | | | ═O)═O)═O)(C([R]) |
| | | | ═O)N[R] |
| 215 | 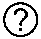 | aMeK(PEG2PEG2g EC18OH) | C[C@](CCCCNC (COCCOCCNC(CO |
| | C42H75N5O13R2 | | CCOCCNC(CC[C |
| | | | @@H](C(O)═O)N |
| | | | C(CCCCCCCCCC |
| | | | CCCCCCC(O)═O) |
| | | | ═O)═O)═O)═O)(C |
| | | | ([R])═O)N[R] |
| 216 | 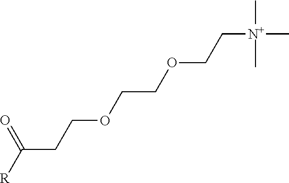 | cPEG3aCO, cPEG3a | C[N+](C)(C)CCOC COCCC([R])═O |
| | C10H21NO3R+ | | |
| 217 | 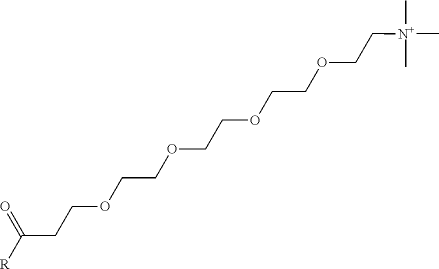 | cPEG5aCO, cPEG5a | C[N+](C)(C)CCOC COCCOCCOCCC ([R])═O |
| | C14H29NO5R+ | | |
| 218 | 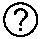 | dFPPEG3F | C[N+](CCOCCOC COc1ccc(C[C@@ |
| | C21H31F2N2O4R2+ | | H](C([R])═O)N[R]) |
| | | | cc1)(CC1)CCC1(F) |
| | | | F |
| 219 | 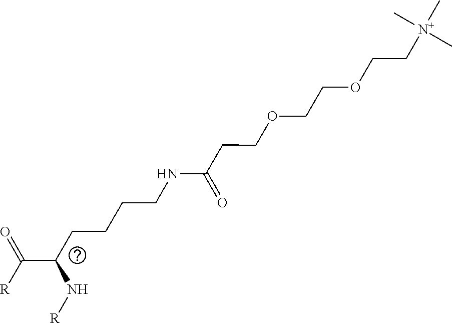 | dK(cPEG3a), k(cPEG3a), dK(cPEG3aCO), k(cPEG3aCO) | C[N+](C)(C)CCOC COCCC(NCCCC [C@H](C([R])═O)N [R])═O |
| | C16H32N3O4R2+ | | |
| 220 | 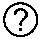 | gEPEG6 | OC([C@H](CCC(N CCOCCOCCOCC |
| | C20H36N2O10R2 | | OCCOCCOCCC |
| | | | ([R])═O)═O)N |
| | | | [R])═O |
| 221 | 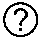 | k(PEG12gEC18OH), dK(PEG12gEC18OH) | OC(CCCCCCCCC CCCCCCCC(N[C |
| | C56H104N4O20R2 | | @@H](CCC(NCC |
| | | | OCCOCCOCCOC |
| | | | COCCOCCOCCO |
| | | | CCOCCOCCOCC |
| | | | OCCC(NCCCC[C |
| | | | @H](C([R])═O)N |
| | | | [R])═O)═O)C(O)═O) |
| | | | ═O)═O |
| 222 | 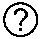 | k(PEG12gEC20OH) dK(PEG12gEC20OH) | OC(CCCCCCCCC CCCCCCCCCC(N |
| | C58H108N4O20R2 | | [C@@H](CCC(NC |
| | | | COCCOCCOCCO |
| | | | CCOCCOCCOCC |
| | | | OCCOCCOCCOC |
| | | | COCCC(NCCCC |
| | | | [C@H](C([R])═O)N |
| | | | [R])═O)═O)C(O) |
| | | | ═O)═O)═O |
| 223 | 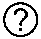 | dK(PEG2PEG2Biotin), k(PEG2PEG2Biotin) | O═C(CCCC[C@@ H]([C@H]1N2)SC |
| | C28H48N6O9SR2 | | [C@@H]1NC2═O) |
| | | | NCCOCCOCC(NC |
| | | | COCCOCC(NCCC |
| | | | C[C@H](C([R])═O) |
| | | | N[R])═O)═O |
| 224 | 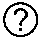 | k (PEG2PEG2C18GolB), | CN(CCOCCOCC (NCCOCCOCC(NC |
| | C40H75N5O11R2 | dK | CCC[C@H](C([R]) |
| | | (PEG2PEG2C18GolB) | ═O)N[R])═O)═O)C |
| | | | (CCCCCCCCCCC |
| | | | CCCCCC(NC(CO) |
| | | | CO)═O)═O |
| 225 | 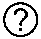 | k(PEG2PEG2C18OH), dK | OC(CCCCCCCCC CCCCCCCC(NCC |
| | C36H66N4O10R2 | (PEG2PEG2C18OH) | OCCOCC(NCCOC |
| | | | COCC(NCCCC[C |
| | | | @H](C([R])═O)N |
| | | | [R])═O)═O)═O)═O |
| 226 | 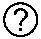 | k(PEG2PEG2Gol AC18OH), | OCC(CO)(C(NCC OCCOCC(NCCOC |
| | C40H73N5O13R2 | dK(PEG2PEG2Gol | COCC(NCCCC[C |
| | | AC18OH) | @H](C([R])═O)N |
| | | | [R])═O)═O)═O)NC |
| | | | (CCCCCCCCCCCC |
| | | | CCCCC(O)═O)═O |
| 227 | 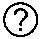 | k(PEG2PEG2PPPg EC18OH) | OC(CCCCCCCCC CCCCCCCC(N[C |
| | C56H94N8O16R2 | dK(PEG2PEG2PPPg | @@H](CCC(N(CC |
| | | EC18OH) | C1)[C@@H]1C(N |
| | | | (CCC1)[C@@H]1C |
| | | | (N(CCC1)[C@@H] |
| | | | 1C(NCCOCCOCC |
| | | | (NCCOCCOCC(N |
| | | | CCCC[C@H](C([R]) |
| | | | ═O)N[R])═O)═O) |
| | | | ═O)═O)═O)═O)C |
| | | | (O)═O)═O)═O |
| 228 | 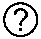 | k(PEG2PEG2Pg EC18OH), | OC(CCCCCCCCC CCCCCCCC(N[C |
| | C46H80N6O14R2 | dK(PEG2PEG2Pg | @@H](CCC(N(CC |
| | | EC18OH) | C1)[C@@H]1C(N |
| | | | CCOCCOCC(NCC |
| | | | OCCOCC(NCCCC |
| | | | [C@H](C([R])═O) |
| | | | N[R])═O)═O)═O) |
| | | | ═O)C(O)═O) |
| | | | ═O)═O |
| 229 | 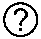 | k(PEG2PEG2Sp6g EC18OH), | C[N+](C)(CCNC(C C[C@@H](C(O)═ |
| | C47H86N7O14R2+ | dK(PEG2PEG2Sp6g | O)NC(CCCCCCC |
| | | EC18OH) | CCCCCCCCCC(O) |
| | | | ═O)═O)═O)CC(NC |
| | | | COCCOCC(NCCO |
| | | | CCOCC(NCCCC |
| | | | [C@H](C([R])═O)N |
| | | | [R])═O)═O)═O |
| 230 | 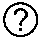 | k(PEG2PEG2Trxg EC18OH), | OC(CCCCCCCCC CCCCCCCC(N[C |
| | C49H86N6O14R2 | dK(PEG2PEG2Trxg | @@H](CCC(NC[C |
| | | EC18OH) | @H](CC1)CC[C@ |
| | | | @H]1C(NCCOCC |
| | | | OCC(NCCOCCOC |
| | | | C(NCCCC[C@H] |
| | | | (C([R])═O)N[R]) |
| | | | ═O)═O)═O)═O)C |
| | | | (O)═O)═O)═O |
| 231 | 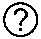 | k(PEG2PEG2gE(C) C12, | CCCCCCCCCCCC (N[C@@H](CCC |
| | C42H78N7O12R2+ | dK(PEG2PEG2gE | (NCCOCCOCC(NC |
| | | (C)C12 | COCCOCC(NCCC |
| | | | C[C@H](C([R])═O) |
| | | | N[R])═O)═O)═O) |
| | | | C(N[C@@H](CC |
| | | | (O)═O)C[N+](C)(C) |
| | | | C)═O)═O |
| 232 | 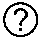 | k(PEG2PEG2gE(C) C18OH, | C[N+](C)(C)C[C@ H](CC(O)═O)NC |
| | C48H88N7O14R2+ | dK(PEG2PEG2gE(C) | ([C@H](CCC(NCCO |
| | | C18OH | CCOCC(NCCOCC |
| | | | OCC(NCCCC[C@ |
| | | | H](C([R])═O)N[R]) |
| | | | ═O)═O)═O)NC(CC |
| | | | CCCCCCCCCCCC |
| | | | CCC(O)═O)═O)═O |
| 233 | 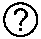 | k(PEG2PEG2gE(c)C12, dK(PEG2PEG2gE | CCCCCCCCCCCC (N[C@@H](CCC |
| | C42H78N7O12R2+ | (c)C12 | (NCCOCCOCC(NC |
| | | | COCCOCC(NCCC |
| | | | C[C@H](C([R])═O) |
| | | | N[R])═O)═O)═O) |
| | | | C(N[C@H](CC(O) |
| | | | ═O)C[N+](C)(C)C) |
| | | | ═O)═O |
| 234 | 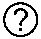 | k(PEG2PEG2gE(c) C18OH, | C[N+](C)(C)C[C@ @H](CC(O)═O)NC |
| | C48H88N7O14R2+ | dK(PEG2PEG2gE(c) | ([C@H](CCC(NCC |
| | | C18OH | OCCOCC(NCCOC |
| | | | COCC(NCCCC[C |
| | | | @H](C([R])═O)N |
| | | | [R])═O)═O)═O)NC |
| | | | (CCCCCCCCCCCC |
| | | | CCCCC |
| | | | (O)═O)═O)═O |
| 235 | 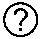 | k (PEG2PEG2gEC10OH), | OC(CCCCCCCCC (N[C@@H](CCC(N |
| | C33H57N5O13R2 | dK | CCOCCOCC(NCC |
| | | (PEG2PEG2gEC10OH) | OCCOCC(NCCCC |
| | | | [C@H](C([R])═O) |
| | | | N[R])═O)═O)═O)C |
| | | | (O)═O)═O)═O |
| 236 | 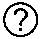 | k (PEG2PEG2gEC12OH | C[N+](C)(C)C[C@ H](CC(O)═O)NC |
| | C42H76N7O14R2+ | (C), | (CCCCCCCCCCC |
| | | dK | (N[C@@H](CCC(N |
| | | (PEG2PEG2gEC12OH | CCOCCOCC(NCC |
| | | (C) | OCCOCC(NCCCC |
| | | | [C@H](C([R])═O) |
| | | | N[R])═O)═O)═O)C |
| | | | (O)═O)═O)═O |
| 237 | 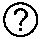 | k(PEG2PEG2gEC12OH (c), | C[N+](C)(C)C[C@ @H](CC(O)═O)NC |
| | C42H76N7O14R2+ | dK | (CCCCCCCCCCC |
| | | (PEG2PEG2gEC12OH | (N[C@@H](CCC(N |
| | | (c) | CCOCCOCC(NCC |
| | | | OCCOCC(NCCCC |
| | | | [C@H](C([R])═O) |
| | | | N[R])═O)═O)═O)C |
| | | | (O)═O)═O)═O |
| 238 | 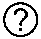 | k(PEG2PEG2gEC16), dK(PEG2PEG2gEC16) | CCCCCCCCCCCC CCCC(N[C@@H] |
| | C39H71N5O11R2 | | (CCC(NCCOCCOC |
| | | | C(NCCOCCOCC |
| | | | (NCCCC[C@H](C |
| | | | ([R])═O)N[R])═O) |
| | | | ═O)═O)C(O)═O)═O |
| 239 | 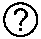 | k (PEG2PEG2gEC16OH), | OC(CCCCCCCCC CCCCCC(N[C@@ |
| | C39H69N5O13R2 | dK(PEG2PEG2g | H](CCC(NCCOCC |
| | | EC16OH) | OCC(NCCOCCOC |
| | | | C(NCCCC[C@H] |
| | | | (C([R])═O)N[R])═O) |
| | | | ═O)═O)C(O)═O) |
| | | | ═O)═O |
| 240 | 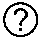 | k(PEG2PEG2gEC18), dK(PEG2PEG2gEC18) | CCCCCCCCCCCC CCCCCC(N[C@@ |
| | C41H75N5O11R2 | | H](CCC(NCCOCC |
| | | | OCC(NCCOCCOC |
| | | | C(NCCCC[C@H] |
| | | | (C([R])═O)N[R])═O) |
| | | | ═O)═O)C(O)═O)═O |
| 241 | 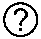 | k (PEG2PEG2gEC18OH | C[N+](C)(C)C[C@ H](CC(O)═O)NC |
| | C48H88N7O14R2+ | (C), | (CCCCCCCCCCCC |
| | | dK | CCCCC(N[C@@H] |
| | | (PEG2PEG2gEC18OH | (CCC(NCCOCCO |
| | | (C) | CC(NCCOCCOCC |
| | | | (NCCCC[C@H](C |
| | | | ([R])═O)N[R])═O) |
| | | | ═O)═O)C(O)═O) |
| | | | ═O)═O |
| 242 | 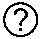 | k(PEG2PEG2gEC18OH (c), | C[N+](C)(C)C[C@ @H](CC(O)═O)NC |
| | C48H88N7O14R2+ | dK | (CCCCCCCCCCC |
| | | (PEG2PEG2gEC18OH | CCCCCC(N[C@@ |
| | | (c) | H](CCC(NCCOCC |
| | | | OCC(NCCOCCOC |
| | | | C(NCCCC[C@H] |
| | | | (C([R])═O)N[R]) |
| | | | ═O)═O)═O)C(O) |
| | | | ═O)═O)═O |
| 243 | 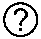 | k (PEG2PEG2gEC18OH), | OC(CCCCCCCCC CCCCCCCC(N[C |
| | C41H73N5O13R2 | dK | @@H](CCC(NCC |
| | | (PEG2PEG2gEC18OH) | OCCOCC(NCCOC |
| | | | COCC(NCCCC[C |
| | | | @H](C([R])═O)N |
| | | | [R])═O)═O)═O)C |
| | | | (O)═O)═O)═O |
| 244 | 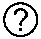 | k (PEG2PEG2gEC20OH), | OC(CCCCCCCCC CCCCCCCCCC(N |
| | C43H77N5O13R2 | dK | [C@@H](CCC(NC |
| | | (PEG2PEG2gEC20OH) | COCCOCC(NCCO |
| | | | CCOCC(NCCCC |
| | | | [C@H](C([R])═O)N |
| | | | [R])═O)═O)═O)C |
| | | | (O)═O)═O)═O |
| 245 | 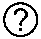 | k(PEG2PEG2gEDAP (C16OH)2), | OC(CCCCCCCCC CCCCCC(NC[C@ |
| | C58H103N7O17R2 | dK(PEG2PEG2gEDAP | @H](C(N[C@@H] |
| | | (C16OH)2) | (CCC(NCCOCCO |
| | | | CC(NCCOCCOCC |
| | | | (NCCCC[C@H](C |
| | | | ([R])═O)N[R])═O) |
| | | | ═O)═O)C |
| | | | (O)═O)═O) |
| | | | NC(CCCCCCCCC |
| | | | CCCCCC(O)═O)═O) |
| | | | ═O)═O |
| 246 | 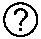 | kPEG2PEG2gEDAP(C 16OH)2;kPEG2PEG2g | C[N+](C)(CCNC(C CCCCCCCCCCCC |
| | C47H86N7O14R2+ | EDap(C16OH)2, | CCCC(O)═O)═O)C |
| | | k(PEG2PEG2gEDAP | C(N[C@@H](CCC |
| | | (C16OH)2), | (NCCOCCOCC(N |
| | | dKPEG2PEG2gEDAP | CCOCCOCC(NCC |
| | | (C16OH)2;dKPEG2PE | CC[C@H](C([R])═O) |
| | | G2gEDap(C16OH)2, | N[R])═O)═O)═O) |
| | | dK(PEG2PEG2gEDAP | C(O)═O)═O |
| | | (C16OH)2) | |
| 247 | 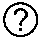 | kPEG2PEG2gEDAP (C16OH)2, | OC(CCCCCCCCC CCCCCCCC(NC[C |
| | C49H86N6O14R2 | dKPEG2PEG2gEDAP | @H](CC1)CC[C@ |
| | | (C16OH)2 | @H]1C(N[C@@H] |
| | | | (CCC(NCCOCCO |
| | | | CC(NCCOCCOCC |
| | | | (NCCCC[C@H](C |
| | | | ([R])═O)N[R])═O) |
| | | | ═O)═O)C(O)═O) |
| | | | ═O)═O)═O |
| 248 | 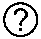 | k(PEG2PEG2gESp6 C18OH), | OC(CCCCCCCCC CCCCCCCCCC(N |
| | C51H90N6O14R2 | dK(PEG2PEG2gESp6 | C[C@H](CC1)CC |
| | | C18OH) | [C@@H]1C(N[C@ |
| | | | @H](CCC(NCCOC |
| | | | COCC(NCCOCCO |
| | | | CC(NCCCC[C@H] |
| | | | (C([R])═O)N[R])═O) |
| | | | ═O)═O)C(O)═O) |
| | | | ═O)═O)═O |
| 249 | 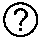 | k(PEG2PEG2gETrx C18OH), | OC([C@H](CCC(N CCOCCOCC(NCC |
| | C40H63N5O14R2 | dK(PEG2PEG2gETrx | OCCOCC(NCCCC |
| | | C18OH) | [C@H](C([R])═O) |
| | | | N[R])═O)═O)═O)N |
| | | | C(CCCCCCCCCO |
| | | | c1cc(C(O)═O)ccc1) |
| | | | ═O)═O |
| 250 | 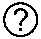 | k(PEG2PEG2gETrx C20OH), | OC([C@H](CCC(N CCOCCOCC(NCC |
| | C40H63N5O14R2 | dK(PEG2PEG2gETrx | OCCOCC(NCCCC |
| | | C20OH) | [C@H](C([R])═O) |
| | | | N[R])═O)═O)═O)N |
| | | | C(CCCCCCCCCO |
| | | | c(cc1)ccc1C(O)═O) |
| | | | ═O)═O |
| 251 | 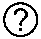 | k(PEG2PEG2gEmXOH), dK | OC(CCCCCCCCC CCCCCCCC(N[C |
| | C46H80N6O14R2 | (PEG2PEG2gEmXOH) | @@H](CCC(N(CC |
| | | | C1)[C@H]1C(NCC |
| | | | OCCOCC(NCCOC |
| | | | COCC(NCCCC[C |
| | | | @H](C([R])═O)N |
| | | | [R])═O)═O)═O)═O) |
| | | | C(O)═O)═O)═O |
| 252 | 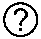 | k(PEG2PEG2gEpXOH), dK | OC(CCCCCCCCC CCCCCCCC(N[C |
| | C56H94N8O16R2 | (PEG2PEG2gEpXOH) | @@H](CCC(N(CC |
| | | | C1)[C@H]1C(N(C |
| | | | CC1)[C@H]1C(N |
| | | | (CCC1)[C@H]1C(N |
| | | | CCOCCOCC(NCC |
| | | | OCCOCC(NCCCC |
| | | | [C@H](C([R])═O)N |
| | | | [R])═O)═O)═O)═O) |
| | | | ═O)═O)C(O)═O) |
| | | | ═O)═O |
| 253 | 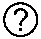 | k(PEG2PEG2pgEC18 OH), | OC(CCCCCCCCC CCCCCCCC(N[C |
| | C50H91N5O17R2 | dK | @@H](CCC(NCC |
| | | (PEG2PEG2pgEC18 | OCCOCCOCCOC |
| | | OH) | COCCOCCC(NCC |
| | | | OCCOCC(NCCCC |
| | | | [C@H](C([R])═O) |
| | | | N[R])═O)═O)═O)C |
| | | | (O)═O)═O)═O |
| 254 | 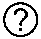 | k(PEG2PEG2pppg EC18OH), | O═C(CCCC[C@@ H]([C@H]1N2)SC |
| | C31H55N5O10SR2 | dK(PEG2PEG2pppg | [C@@H]1NC2═O) |
| | | EC18OH) | NCCOCCOCCOC |
| | | | COCCOCCOCCC |
| | | | (NCCCC[C@H](C |
| | | | ([R])═O)N[R])═O |
| 255 | 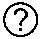 | k (PEG2PEG6gEC18OH), | CC(NCCOCCOCC OCCOCCOCCOC |
| | C35H67N3O15R2 | dK | COCCOCCOCCO |
| | | (PEG2PEG6gEC18OH) | CCOCCOCCC(NC |
| | | | CCC[C@H](C([R]) |
| | | | ═O)N[R])═O)═O |
| 256 | 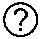 | k(dPEG12AcBr), dK(dPEG12AcBr) | CC(NCCOCCOCC OCCOCCOCCOC |
| | C23H43N3O9R2 | | CC(NCCCC[C@H] |
| | | | (C([R])═O)N[R]) |
| | | | ═O)═O |
| 257 | 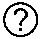 | k(dPEG12AcVitE), dK(dPEG12AcVitE) | O═C(CCOCCOCC OCCOCCOCCOC |
| | C23H42BrN3O9R2 | | CNC(CBr)═O)NCC |
| | | | CC[C@H](C([R])═ |
| | | | O)N[R] |
| 258 | 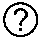 | k(dPEG6Ac), dK(dPEG6Ac) | CC(NCCOCCOCC OCCOCCOCCOC |
| | C29H55N3O12R2 | | COCCOCCOCCC |
| | | | (NCCCC[C@H](C |
| | | | ([R])═O)N[R])═O) |
| | | | ═O |
| 259 | 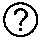 | k(dPEG6AcBr), dK(dPEG6AcBr) | O═C(CCOCCOCC OCCOCCOCCOC |
| | C29H54BrN3O12R2 | | COCCOCCOCCN |
| | | | C(CBr)═O)NCCCC |
| | | | [C@H](C([R])═O) |
| | | | N[R] |
| 260 | 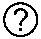 | k(dPEG9Ac), dK(dPEG9Ac) | CC(C)CCC[C@@ H](C)CCC[C@@H] |
| | C57H98N4O14R2 | | (C)CCC[C@](C) |
| | | | (CC1)Oc(c(C)c2C)c |
| | | | 1c(C)c2OCC(N[C |
| | | | @@H](CCC(NCC |
| | | | OCCOCCOCCOC |
| | | | COCCOCCC(NCC |
| | | | CC[C@H](C([R]) |
| | | | ═O)N[R])═O)═O)C |
| | | | (O)═O)═O |
| 261 | 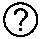 | mPEG12CO | COCCOCCOCCO CCOCCOCCOCC |
| | C26H51O13R | | OCCOCCOCCOC |
| | | | COCCC([R])═O |
| 262 | 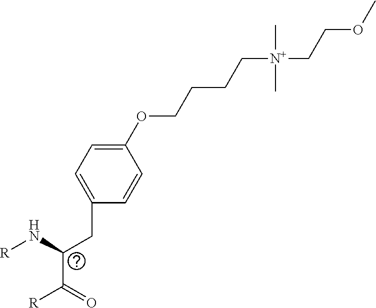 | mPEG2TMA4F | C[N+](C)(CCCCO c1ccc(C[C@@H] (C([R])═O)N[R])cc1) CCOC |
| | C18H29N2O3R2+ | | |
| 263 | 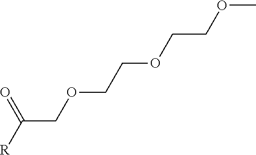 | mPEG3CO | COCCOCCOCC ([R])═O |
| | C7H13O4R | | |
| 264 | 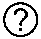 | mPEG6CO | COCCOCCOCCO CCOCCOCCC |
| | C14H27O7R | | ([R])═O |
| 1 | | | |

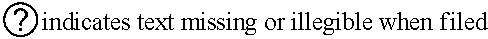

### VIII. Examples

The following examples illustrate the invention. These examples are not intended to limit the scope of the present invention, but rather to provide guidance to the skilled artisan to prepare and use the compounds, compositions, and methods of the present invention. While particular aspects of the present invention are described, the skilled artisan will appreciate that various changes and modifications can be made without departing from the spirit and scope of the invention.

### General Peptide Synthetic Procedure 1

IL-23R inhibitor compounds described herein were synthesized from amino acids monomers using standard Fmoc-based solid phase synthesis on various instruments such as Protein Technology's Symphony multiple channel synthesizer and CEM microwave peptide synthesizer. The peptides were assembled using various coupling conditions such as HBTU (0-Benzotriazole-N,N,N′,N′-tetramethyl-uronium-hexafluoro-phosphate) and diisopropylethylamine(DIEA), Oxyma/DIC, or PyAOP(7-Azabenzotriazol-1-yloxy)tripyrrolidinophosponium hexafluorophosphate) and DIEA. Rink Amide MBHA resin was used for peptides with C-terminal amides and pre-loaded Wang Resin with N-α-Fmoc protected amino acid or 2-chlorotrityl resin was used for peptide with C-terminal acids. Peptide inhibitors of the present invention were identified based on medical chemistry optimization and/or phage display and screened to identify those having superior binding and/or inhibitory properties.

### Preparation of Certain Modified Amino Acids

Certain modified amino acids appear in the sequences of the IL-23R inhibitors described herein. Those modified amino acids, and their precursors suitable for synthesizing the inhibitors described herein may be obtained from commercial sources, synthesized as described in the art, or by any suitable route. For example, substituted tryptophans may be prepared by any suitable route. Preparation of certain substituted tryptophans including those substituted at the seven position, such as 7-alkyl-tryptophans (e.g., 7-ethyl-L-tryptophans), which along with other substituted tryptophans, are described in, for example WO 2021/146441 A1. The synthesis of certain additional modified amino acids are described herein below.

### a. Synthesis of (S)-5-(4-(2-((((9H-fluoren-9-yl)methoxy)carbonyl)amino)-2-carboxyethyl)phenoxy)-N,N,N-trimethylpentan-1-aminium (TMAPF)

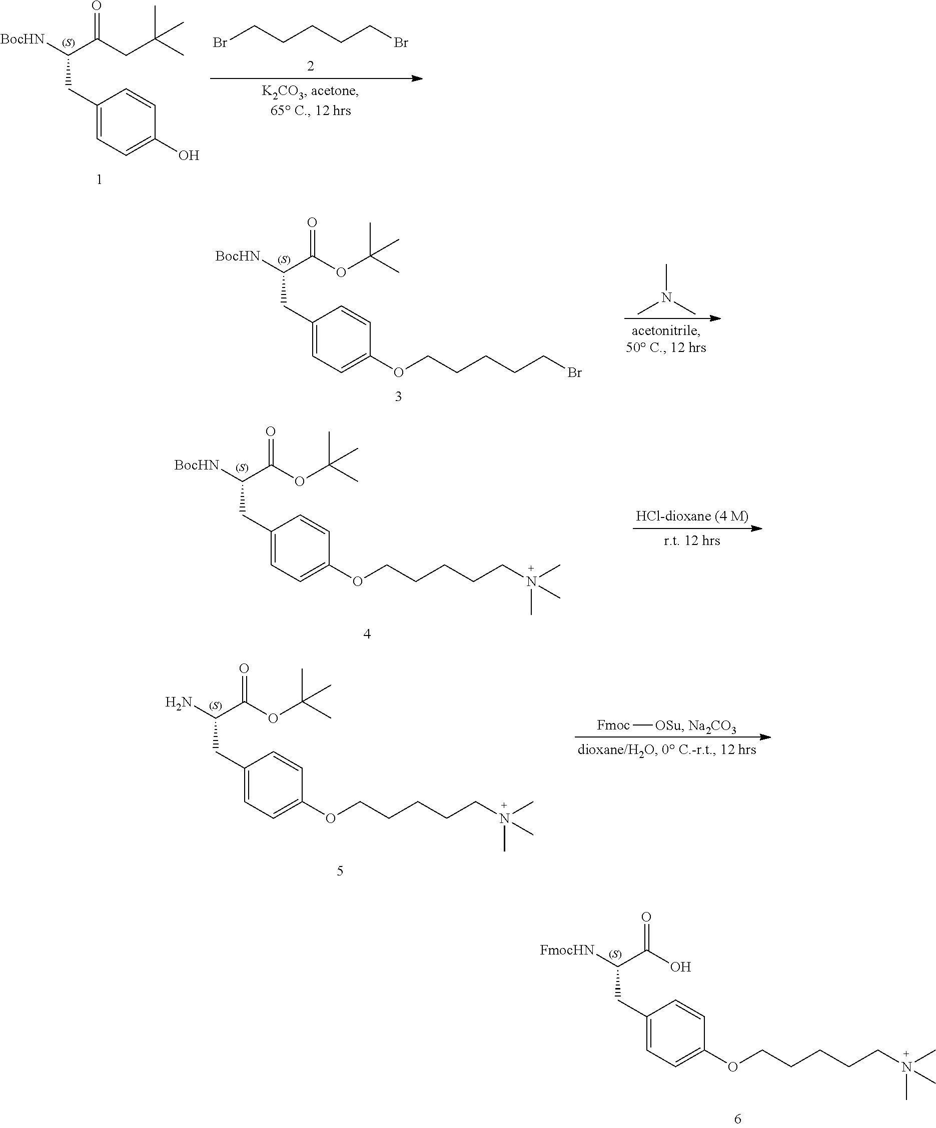

To a mixture of 1 (6.60 g, 19.7 mmol), K₂CO₃ (4.09 g, 29.6 mmol) and acetone (50 mL) was added 2 (4.99 g, 21.7 mmol). The reaction mixture was heated to refluxed and stirred for 12 hours. The reaction mixture was poured into water (500 mL) and extracted with ethyl acetate (500 mL×3). The combined organic extracts were washed with brine (500 mL), dried over anhydrous Na₂SO₄, filtered and concentrated under reduced pressure to afford the crude product, which was purified by FCC (eluent: petroleum ether:ethyl acetate=1:0 to 5:1) to afford crude product 3 (5.26 g, yield: 54.8%) as pale colorless oil. MS (ESI): mass calculated for C₂₃H₃₆BrNO₅, 486.44, m/z found 509.9 [M+23]+. ¹H NMR (400 MHz, CDCl₃): δ ppm 7.07 (d, J=8.4 Hz, 2H), 6.81 (d, J=8.6 Hz, 2H), 4.97 (br d, J=8.2 Hz, 1H), 4.36-4.48 (m, 1H), 3.95 (t, J=6.3 Hz, 2H), 3.45 (t, J=6.8 Hz, 2H), 3.00 (br d, J=3.7 Hz, 2H), 1.87-2.01 (m, 2H), 1.76-1.86 (m, 2H), 1.62-1.69 (m, 2H), 1.42 (d, J=2.8 Hz, 18H).

To a mixture of 3 (5.26 g, 10.8 mmol) in acetonitrile (50 mL) was added trimethylamine in acetonitrile (2 M, 8.11 mL). The reaction mixture was stirred for 12 hours at 50° C. The reaction mixture was concentrated under reduced pressure to obtain the product 4 (5.0 g, yield: 99.3%) as pale yellow solid.

MS (ESI): mass calculated for C₂₆H₄₅N₂O₅, 465.646, m/z found 465.2 [M]⁺. The mixture of 4 (4.00 g, 8.59 mmol) in 4M HCl-dioxane (43.0 mL, 172 mmol) was stirred for 12 hours at room temperature. The solvent was removed under reduced pressure to obtain the product 5 (3.00 g, yield: crude) as a white solid, which was used to next step directly. MS (ESI): mass calculated For C₁₂H₂₉N₂O₃, 309.424, m/z found 309.1 [M+H]⁺.

Compound 5 (3.00 g, 8.67 mmol) was dissolved in dioxane (20 mL) and water (20 mL) in a round-bottom flask. Na₂CO₃ (1.38 g, 13.0 mol) was added, and the solution cooled to 0° C. in an ice bath. Then Fmoc-OSu (3.22 g, 9.54 mol) was dissolved in dioxane (20 mL) and added in portions to the solution at 0° C. The reaction was stirred for 2 hours at 0° C. The reaction was allowed to warm to room temperature overnight. The reaction was acidified with 2N HCl (50 mL). The reaction mixture was purified by preparative HPLC using a Xtimate C18 150*40 mm*5 um (eluent: 20% to 50% (v/v) CH₃CN and H₂O with 0.05% HCl) to afford product. The product was suspended in water (40 mL), the mixture frozen using dry ice/ethanol, and then lyophilized to dryness to afford the title compound 6 (TMAPF, 3.57 g, yield: 61.9%, purity: 99.2%) as pale yellow solid. MS (ESI): mass calculated For C32H₃₉N₂O₅, 531.662, m/z found 531.4 [M+H]⁺. ¹H NMR (400 MHz, DMSO-d₆)6 ppm 7.89 (d, J=7.6 Hz, 2H), 7.73 (d, J=8.2 Hz, 1H), 7.65 (t, J=7.2 Hz, 2H), 7.39-7.43 (m, 2H), 7.27-7.34 (m, 2H), 7.19 (d, J=8.2 Hz, 2H), 6.78-6.89 (m, 2H), 4.06-4.25 (m, 4H), 3.84-3.99 (m, 2H), 3.25-3.37 (m, 2H), 3.05 (s, 9H), 3.00 (d, J=4.0 Hz, 1H), 2.70-2.84 (m, 1H), 1.63-1.82 (m, 4H), 1.30-1.46 (m, 2H)

### b. Synthesis of (S)-2-((((9H-fluoren-9-yl)methoxy)carbonyl)amino)-3-(7-(3-acetamidophenyl)-1H-indol-3-yl)propanoic acid (7-(3-Nacetyl-phenyl)-tryptophan or 7(3NAcPh)W)

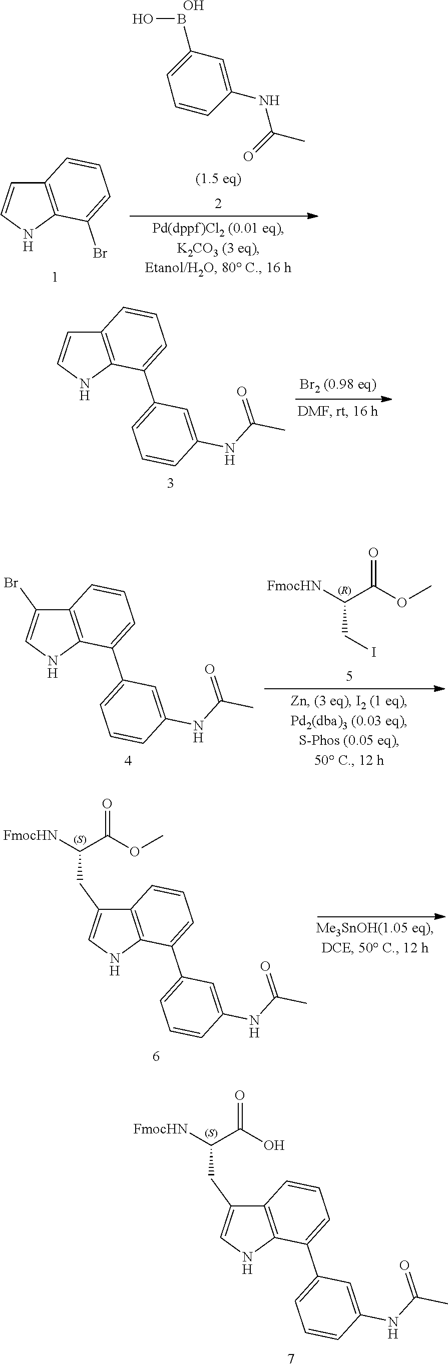

To a solution of 1 (30.0 g, 153 mmol), compound 2 (41.1 g, 230 mmol) and K₃PO₄ (97.4 g, 459 mmol) in H₂O/ethanol (500 mL) and, Pd(dppf)Cl₂ (1.12 g, 1.53 mmol) was added under an N₂ atmosphere. The mixture was stirred at 80° C. for 16 h. The mixture was filtered. The mixture was concentrated, then extracted with ethyl acetate (500 mL×2), dried with anhydrous Na₂SO₄. The organic layer was concentrated and purified by FCC (eluent: petroleum ether/ethyl acetate=1:0 to 55:45) to give 3 (25.0 g, yield: 62.5%) as yellow oil MS (ESI): mass calculated for C₁₆H₁₄N₂O, 250.295, m/z found 251.0 [M+].

To a 1 L round-bottomed flask containing a solution of 3 (12.0 g, 47.9 mmol) in DMF (300 mL) bromine (Br₂, 2.422 mL, 47.0 mmol) was slowly added. The mixture was stirred at 25° C. for 16 hours. The solution was added to aqueous sodium sulfite (500 mL), the mixture was stirred at 25° C. for 2 hours. The mixture was filtered, the filter cake was mixed with H₂O (400 mL) and stirred at 25° C. for 1 h. The mixture was filtered, the solid was collected to give 4 as a crude product, which was purified by preparative high-performance liquid chromatography (Column: Phenomenex C18 250×50 mm×10 um, Condition: water (FA)-CAN (20%-60%)). The mixture was concentrated, extracted with CH₂Cl₂ (1 L×2), washed with brine, dried with anhydrous Na₂SO₄. The organic layers was filtered and concentrated to give 4 (9.70 g, yield: 60.8%) as a pale white. MS (ESI): mass calculated For C₁₆H₁₃BrN₂O, 329.191, m/z found 328.8 [M].

A 250 mL three neck round-bottomed flask was charged with activated Zn powder (5.84 g, 89.3 mmol), DMF (120 mL) and I₂ (382 mg, 1.50 mmol) was added under an N₂ atmosphere at room temperature. After stirring for 20 min, a solution of 5 (13.6 g, 30.1 mmol) in DMF (30 mL) was added to the mixture. The reaction mixture was stirred for 30 min. at room temperature, after which 4 (9.70 g, 29.5 mmol), tris(dibenzylideneacetone)palladium (826 mg, 0.902 mmol), 2-dicyclohexylphosphino-2′,6′-dimethoxybiphenyl (617 mg, 1.50 mmol) were added under an N₂ atmosphere. The reaction mixture was stirred at 50° C. for 12 hours, after which solvent was removed under reduced pressure to give crude product 6. The crude product was extracted with ethyl acetate (1500 mL). The extract was washed with H₂O (500 mL×2), followed by brine (500 mL), after which it was dried over anhydrous Na₂SO₄, filtered, and concentrated to dryness in vacuo to give crude intermediate 6, which was purified by silica gel chromatography (0-100% ethyl acetate/petroleum ether (EtOAc/PE)) to afford 6 (11.0 g, yield: 63.8%) as a brown-yellow oil. MS (ESI): mass calculated for C₃₅H₃₁N₃O₅, 573.638, m/z found 574.1 [M+1].

Intermediate 6 (11.0 g, 19.2 mmol), a stir bar, Me₃SnOH (3.64 g, 20.1 mmol) and DCE (150 mL) were added to a 250 mL round-bottomed flask and stirred at 50° C. for 12 hours. To the reaction mixture 2 N HCl was added to adjust the pH to 6. A second reaction series starting with a solution of 1 was prepared and the combined reaction mixtures were concentrated under reduced pressure to give the crude product 7, which was purified by preparative HPLC using a Xtimate C18 150×40 mm×5 um (eluent: 38% to 68% (v/v) CH₃CN and H₂O with 0.05% HCl) to afford product 7. The product was suspended in water (100 mL), the mixture frozen using dry ice/ethanol, and then lyophilized to dryness to afford 7 (7(3NAcPh)W, 11.8 g, yield: 66.8%) as a white solid. MS (ESI): mass calculated For C₃₄H₂₉N₃O₅, 559.611, m/z found 560.0 [M+1]. ¹H NMR DMSO-d₆ (400 MHz) δ 10.73 (s, 1H), 10.10 (s, 1H), 7.52-8.02 (m, 7H), 6.96-7.52 (m, 9H), 4.03-4.44 (m, 3H), 3.25 (d, J=13.2 Hz, 2H), 3.01-3.15 (m, 1H), 2.08 (s, 3H).

### c. Synthesis of (S)-2-((((9H-fluoren-9-yl)methoxy)carbonyl)amino)-3-(6-(tert-butoxy)naphthalen-2-yl)propanoic acid (5-methyl-pyridyl-alanine or 5MePyridinAla

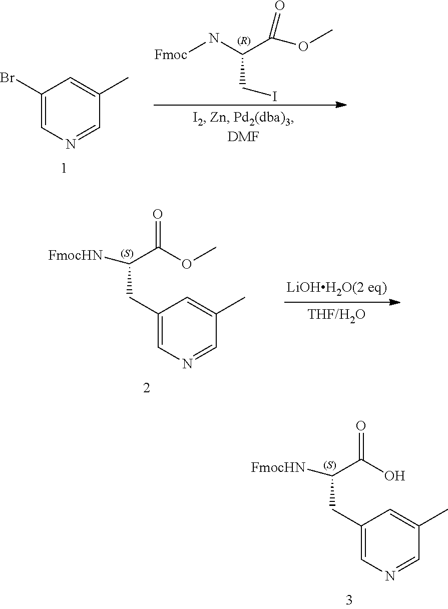

Activated Zn powder (8.18 g, 125 mmol), DMF (150 mL) and I₂ (0.534 g, 2.11 mmol) were stirred under an N₂ atmosphere at room temperature for 20 min, after which (R)-methyl 2-((((9H-fluoren-9-yl)methoxy)carbonyl)amino)-3-iodopropanoate (19.0 g, 42.1 mmol) in DMF (25 mL) was added. The reaction mixture was stirred for 30 min at room temperature, after which a mixture of 1 (7.97 g, 46.3 mmol), tris(dibenzylideneacetone)palladium (1.16 g, 1.26 mmol) and 2-dicyclohexylphosphino-2′,6′-dimethoxybiphenyl (0.864 g, 2.11 mmol) in DMF (25 mL) was added under an N₂ atmosphere. The resulting reaction mixture was stirred at 50° C. for 12 h. The solvent was removed under reduced pressure to give the crude, which was purified by FCC (eluent: petroleum ether:ethyl acetate=1:0 to 0:1 and ethyl acetate: methanol=1:0 to 2:1) to afford the product 2 (10.00 g, 57.0% yield) as pale yellow liquid. MS (ESI): mass calculated for C₂₅H₂₄N₂O₄, 416.469, m/z found 417.1 [M+H]⁺.

To a mixture of 2 (9.50 g, 22.8 mmol) in THF (100 mL) was added LiOH·H₂O (1.91 g, 45.6 mmol) in H₂O (10 mL). The mixture was stirred for 1 h at 0° C. TLC showed most SM were consumed. To the reaction mixture was added HCl (1 N) dropwise at ice bath to pH=5. The reaction mixture was concentrated under reduced pressure, then poured into water (200 mL) the mixture was extracted with THF (200 mL×3). The organic layers were combined, washed with brine (100 mL), dried over anhydrous Na₂SO₄. After filtering the organic layers were concentrated under reduced pressure to afford crude product 3, which was purified by FCC (eluent: ethyl acetate: methanol=1:0 to 2:1) to obtain 3 (SMePyridinAla, 6.716 g, yield: 72.3%) as a white powder. MS (ESI): mass calculated For C₂₄H₂₂N₂O₄, 402.442, m/z found 403.1 [M+H]⁺. ¹H NMR DMSO-d₆ (Bruker_400 MHz): δ 8.18 (s, 2H), 7.88 (d, J=7.6 Hz, 2H), 7.63 (d, J=7.2 Hz, 2H), 7.45-7.26 (m, 5H), 6.81 (s, 1H), 4.33-4.21 (m, 1H), 4.20-4.09 (m, 2H), 3.95 (s, 1H), 3.06-3.05 (m, 1H), 2.92-2.89 (m, 1H), 2.18 (s, 3H).

### d. Synthesis of (S)-2-((((9H-fluoren-9-yl)methoxy)carbonyl)amino)-3-(4-(2-(3-((2,2,4,6,7-pentamethyl-2,3-dihydrobenzofuran-5-yl)sulfonyl)guanidino)ethoxy) phenyl)propanoic acid (AEF(G))

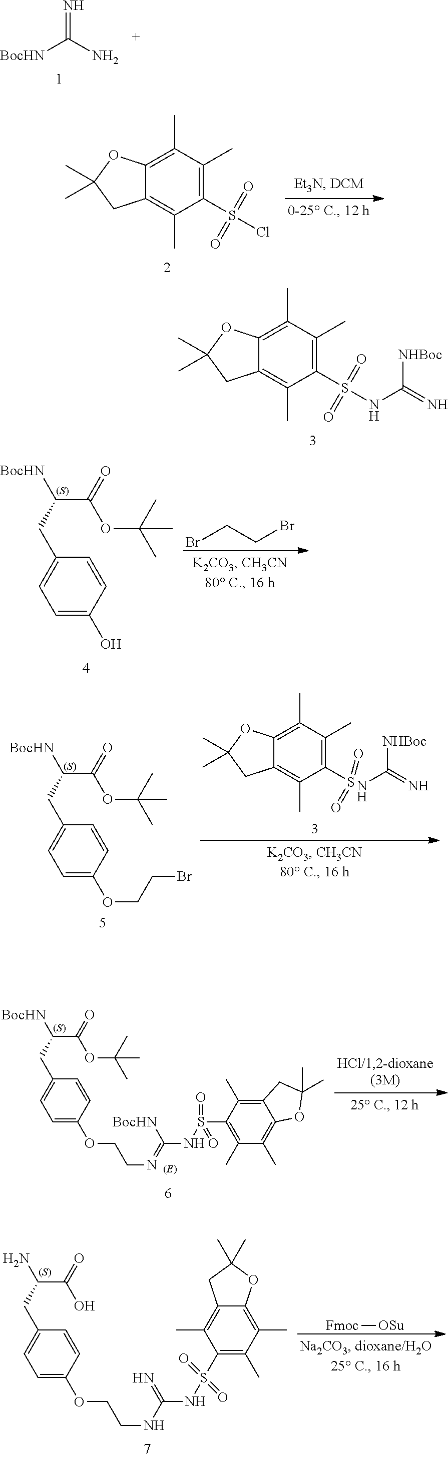

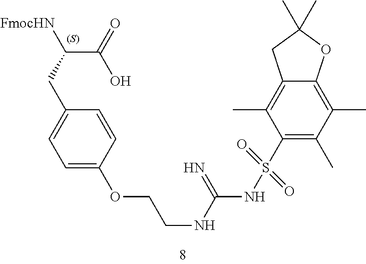

Starting material 1 (9.9 g, 62.2 mmol), a stir bar, Et₃N (14 mL, 101 mmol), and dichloromethane (DCM, 250 mL) were added to a 500 mL round-bottomed flask. The resulting mixture was treated with 2 (10 g, 34.6 mmol) in portions under ice-water bath. Then the reaction mixture was stirred at 25° C. for 12 hours. The reaction mixture was diluted with H₂O (800 mL), extracted with DCM (400 mL×2). The organic phase extracts were combined, washed with brine (800 mL), and concentrated to give the crude intermediate 3 as a yellow solid. The crude intermediate was triturated with ethyl acetate (50 mL) and the suspension isolated via filtration. The filter cake was washed with ethyl acetate (20 mL×3) before drying under reduced pressure to give the 3 (7.12 g, 49%) as a white solid. MS (ESI): mass calculated for C₁₉H₂₉N₃O₅S6, 411.5, m/z found 412.1 [M+H]⁺.

Starting material 4 (50.0 g, 148 mmol), a stir bar, DMF (300 mL), and K₂CO₃ (102 g, 739 mmol) were added to a nitrogen-purged 1000 mL round-bottomed flask. The flask was subsequently evacuated and refilled with nitrogen (×3), after which 1,2-dibromoethane (154 mL, 1.78 mol) was added, and the resulting mixture was stirred at 80° C. for 16 h under a N₂ atmosphere. The reaction mixture was filtered and concentrated to dryness under reduced pressure to give the crude product, which was subjected to silica gel chromatography (eluent: EtOAc: pet ether=0-60%) to give the 5 (64 g, 96%) as a light yellow oil. MS (ESI): mass calculated for C₂₀H₃₀BrNO₅, 444.36, m/z found 466.1 [M+Na]⁺.

Intermediate 5 (6.1 g, 13.7 mmol), 3 (6.2 g, 15.1 mmol), K₂CO₃ (7.6 g, 55.0 mmol), a stir bar, and CH₃CN (100 mL) were charged into a 250 mL round-bottomed flask. The reaction mixture was stirred at 80° C. for 16 h under a N₂ atmosphere. The reaction mixture was cooled to room temperature, diluted with H₂O (200 mL), extracted with ethyl acetate (100 mL×2). The organic phases were combined and washed with brine (300 mL) and concentrated to give the crude intermediate 6. The crude intermediate was purified by flash column chromatography (FCC, eluent: ethyl acetate/petroleum ether=0:1 to 2:1) to give the 6 (6.62 g, 44.2%) as a white solid. MS (ESI): mass calculated for C₃₉H₅₈N₄O₁₀S, 774.9, m/z found 775.5 [M+H]⁺.

Intermediate 6 (6.6 g, 8.52 mmol), HCl/1, 4-dioxane (90 mL, 4M), a stir bar, and 1, 4-dixoane (30 mL) were charged into a 250 mL round bottomed flask. The resulting mixture was stirred at 25° C. for 12 hr. The solvent was removed under reduced pressure to give intermediate 7 (7.8 g, crude product) as a colourless oil, which was directly used to next step. MS (ESI): mass calculated for C₂₅H₃₄N₄O₆S, 518.6, m/z found 519.2 [M+H]⁺.

Intermediate 7 (7.80 g, 15.0 mmol), a stir bar, Na₂CO₃ (3.19 g, 30.1 mmol), Fmoc-OSu (5.58 g, 16.5 mmol), 1, 4-dioxane (50 mL), and H₂O (50 mL) were added into a 250 mL round-bottomed flask at 25° C. The reaction mixture was stirred at 25° C. for 16 hours, after which it was adjusted to pH=5-6 with HCl (2M) and the resulting reaction mixture was extracted with EtOAc (150 mL×3). The organic phases from the extraction were combined and washed with brine (200 mL) and concentrated to give the crude intermediate 7. The crude intermediate was purified by preparative HPLC with a Column: Phenomenex C18 150×40 mm×5 um, (eluent: 42% to 72% (v/v) CH₃CN and H₂O with 0.1% HCl) to afford pure product. The product was suspended in water (100 mL), the mixture frozen using dry ice/ethanol, and then lyophilized to dryness to afford desired product 8 (AEF(G), 4 g, 36%) as a white solid. MS (ESI): mass calculated for C₄₀H₄₄N₄O₈S, 740.9, m/z found 741.3 [M+H]+. ¹H NMR (400 MHz, DMSO-d₆): 7.87 (d, J=7.2 Hz, 2H), 7.71-7.62 (m, 2H), 7.39 (td, J=4.0, 7.2 Hz, 2H), 7.29 (td, J=7.6, 12.0 Hz, 2H), 7.14 (br d, J=8.0 Hz, 2H), 6.99-6.85 (m, 1H), 6.77 (br d, J=8.4 Hz, 2H), 6.59-6.50 (m, 1H), 4.21-4.06 (m, 4H), 3.88 (br s, 2H), 3.42-3.36 (m, 4H), 2.99 (br dd, J=4.4, 14.0 Hz, 1H), 2.92 (s, 2H), 2.78 (br dd, J=10.8, 13.6 Hz, 1H), 2.47 (br s, 3H), 2.41 (s, 3H), 1.97 (s, 3H), 1.38 (s, 6H).

### e. Synthesis of 2-(2-(2-carboxyethoxy)ethoxy)-N,N,N-trimethylethan-1-aminium (cPEG3a)

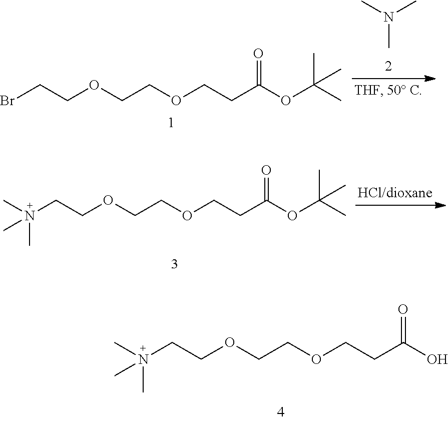

A mixture 1 (5.00 g, 16.8 mmol) and trimethylamine 2 (25 mL, 50 mmol, in THF) in dry THF (10 mL) was stirred for 16 hours at 50° C. under N2. The mixture was concentrated to give the product 3 (6.0 g, yield: 99.8%) as yellow oil. ¹H NMR (DMSO-d₆, 400 MHz): δ3.88-3.79 (m, 2H), 3.64-3.48 (m, 8H), 3.12 (s, 9H), 2.42 (t, J=6.4 Hz, 2H), 1.39 (s, 9H). A mixture of 3 (6.00 g, 16.8 mmol) and HCl/dioxane (60 mL, 240 mmol) was stirred for 16 hours at 25° C. under N₂. The mixture was concentrated to give the product 4 (cPEG3a, 4.3 g, yield: 99.8%) as yellow oil. ¹H NMR (D₂O, 400 MHz): δ 3.96-3.87 (m, 2H), 3.74 (t, J=5.6 Hz, 2H), 3.64 (s, 4H), 3.57-3.49 (m, 2H), 3.12 (s, 9H), 2.60 (t, J=5.6 Hz, 2H).

### f. Synthesis of (S)-2-(2-(2-(4-(2-((((9H-fluoren-9-yl)methoxy)carbonyl) amino)-2-carboxyethyl)phenoxy)ethoxy)ethoxy)-N,N,N-trimethylethan-1-aminium (APEG3F)

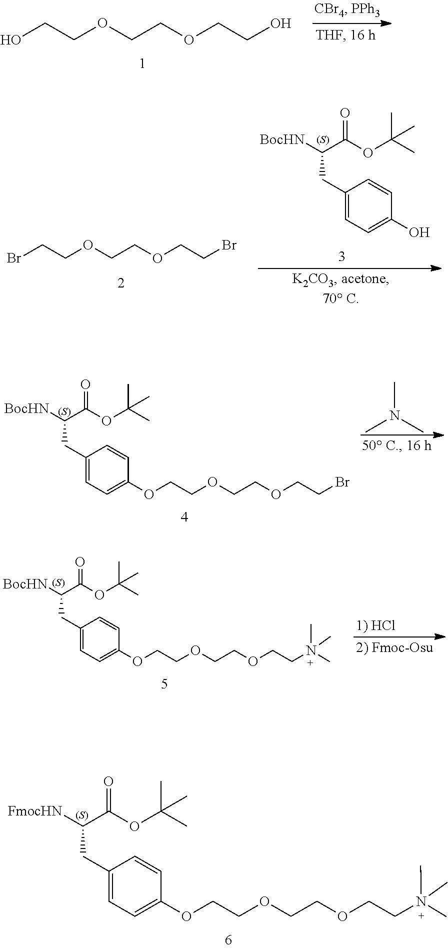

To a mixture of 1 (50.0 g, 333 mmol) in THF (1.3 L) was added PPh3 (188 g, 716 mmol), after which CBr4 (243 g, 732 mmol) was very slowly added to the mixture at 0° C. The mixture was stirred at room temperature overnight (16 h) and then concentrated under reduced pressure to give the crude intermediate 2. Petroleum ether (2.0 L) and ethyl acetate (200 mL) were added to the mixture and stirred at 25° C. for 0.5 h. The mixture was filtered, concentrated under reduced pressure, and purified by FCC (eluent: petroleum ether:ethyl acetate=1:0 to 1:9) to give intermediate 2 (52 g, yield: 56.6%) as colorless oil. ¹H NMR (400 MHz, Chloroform-d): 3.91-3.81 (m, 4H), 3.75-3.68 (m, 4H), 3.55-3.46 (m, 4H).

To a solution of 3 (45.9 g, 136 mmol) and K2CO3 (56.3 g, 408 mmol) in acetone (1 L) was added 2 (75.0 g, 272 mmol) under a nitrogen atmosphere. The mixture was stirred at 70° C. for 16 h. The mixture was filtered and evaporated, and the residue was purified by flash column chromatography FCC (eluent: petroleum ether:ethyl acetate=1:0 to 1:9) to give the intermediate 4 (45 g, yield: 61.6%) as a pale yellow oil. MS (ESI): mass calculated for C₂₄H₃₈BrNO₇, 532.47, m/z found 433.8 [M−100]⁺.

A solution of 4 (51 g, 96 mmol) in trimethylamine (239 mL, 2 M, in THF), was stirred at 50° C. for 16 h. The mixture was concentrated under reduced pressure to give the crude intermediate 5 (56 g, crude) as pale yellow oil, which was used in the next step without purification. MS (ESI): mass calculated for C27H47N2O7+, 511.67, m/z found 511.4 [M]+

A mixture of 5 (56.0 g, 94.7 mmol) in HCl/dioxane (592 mL, 4 M) was stirred at 25° C. for 16 h, after which it was concentrated under reduced pressure, dissolved in H₂O (200 mL), and quenched with an aqueous solution of Na₂CO₃ at 0° C. to adjust pH=7. Then Na₂CO₃ (15.0 g, 142 mmol) and Fmoc-OSu (31.9 g, 94.4 mmol) in acetone (150 mL) were added under a nitrogen atmosphere and stirred at 25° C. for 3 h. The mixture was acidified with 2 M HCl, adjusted to pH=4 and concentrated under reduced pressure. The mixture was extracted with ethyl acetate (300 mL×2). The aqueous phase was concentrated under reduced pressure to give crude product 6 (H₂O solution), which was purified by preparative HPLC using a Phenomenex Gemini Xtimate C18 150*40 mm*5 um, 100 Å (eluent: 53% to 83% (v/v) water (0.225% FA)-ACN) to afford the title compound 6 (APEG3F, 43 g, yield: 78.8%) as an off-white solid. MS (ESI): mass calculated for C₁₈H₃₁N₂O₅⁺, 355.45, m/z found 355.1 [M]⁺. ¹H NMR (400 MHz, DMSO-d₆) δ 8.40 (s, 1H), 7.88 (d, J=7.6 Hz, 2H), 7.66 (d, J=7.2 Hz, 2H), 7.44-7.36 (m, 2H), 7.31 (q, J=7.2 Hz, 2H), 7.18-7.04 (m, 3H), 6.77 (d, J=8.4 Hz, 2H), 4.24-4.13 (m, 3H), 4.00 (d, J=3.6 Hz, 3H), 3.81 (s, 2H), 3.73-3.67 (m, 2H), 3.58 (s, 4H), 3.54-3.48 (m, 2H), 3.07 (s, 9H), 3.05-2.98 (m, 1H), 2.85-2.76 (m, 1H).

### f. Synthesis of N2-(((9H-fluoren-9-yl)methoxy)carbonyl)-N4,N4-dimethyl-L-asparagine (N(N(Me)2)

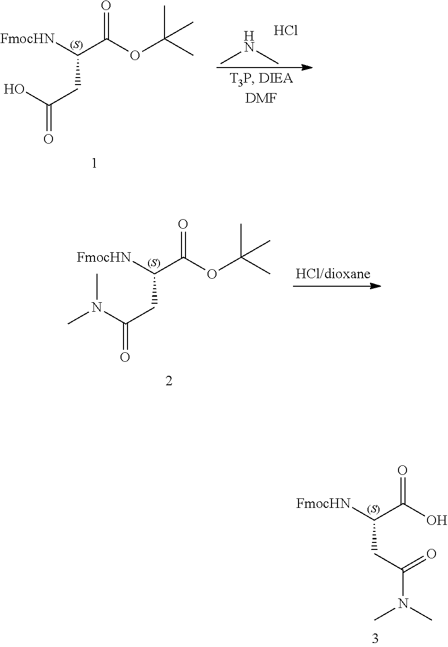

To a solution of starting material 1 (50 g, 122 mmol), dimethylamine (10.9 mg, 134 mmol), and diisopropyl ethyl amine (DIEA, 62.0 g, 365 mmol) in DMF (200 mL) at 0° C. was degassed with N₂ three times and propylphosphonic anhydride (T₃P®, 109 g, 182 mmol) was added via syringe. The mixture was stirred at 20° C. for 12 hours after which it was poured into ice water (500 mL) and extracted with ethyl acetate (500 mL×3). The combined organic extracts were washed with brine, dried over anhydrous Na₂SO₄, filtered, and concentrated under reduced pressure to afford the crude intermediate 2, which was purified by fast column chromatography (FCC, eluent: petroleum ether:ethyl acetate=1:0 to 1:2) to afford 2 (45 g, yield: 84.4%) as pale yellow solid. MS (ESI): mass calculated for C₂₅H₃₀N₂O₅, 438.52, m/z found 439.2 [M+H]⁺.

Intermediate 2 (45 g, 103 mmol) was stirred in HCl/dioxane (1 L, 4 M) at 20° C. for 16 h. The reaction mixture was filtered and concentrated. EtOAc (200 mL) was added to the concentrated material after which petroleum ether (200 mL) was added dropwise. The mixture was stirred at 20° C. for 3 h resulting in a solid that was filtered to afford 3 (N(N(Me)2), 25 g, yield: 62.3%) as white solid. MS (ESI): mass calculated for C₂₁H₂₂N₂O₅, 382.41, m/z found 383.1 [M+H]+. ¹H NMR (DMSO-d₆, 400 MHz): δ ppm 12.59 (s, 1H), 7.86 (d, J=7.6 Hz, 2H), 7.67 (d, J=7.2 Hz, 2H), 7.43-7.21 (m, 5H), 4.39-4.31 (m, 1H), 4.29-4.23 (m, 2H), 4.21-4.15 (m, 1H), 2.90 (s, 3H), 2.78 (s, 3H), 2.75-2.62 (m, 2H).

### 2. Synthesis of N2-(((9H-fluoren-9-yl)methoxy)carbonyl)-N6-acetyl-N6-methyl-L-lysine (Lysine N-(MeAc) or K(NMAcO)

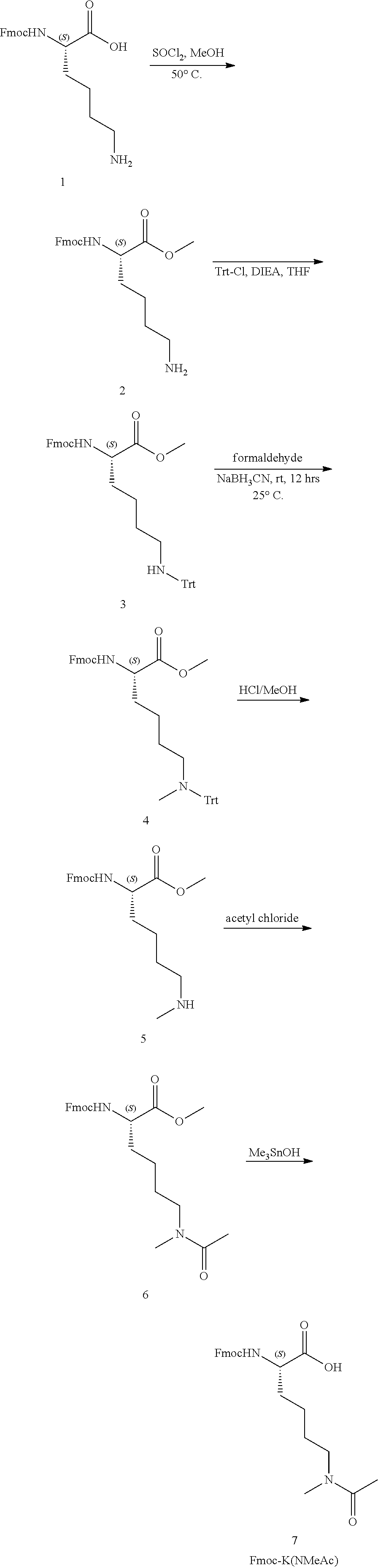

Starting material 1 (21 g, 57.0 mmol) and MeOH (300 mL) were combined in a flask under a N₂ atmosphere. Thionyl chloride (8.14 g, 68.4 mmol) was added to the flask dropwise over 15 minutes at a temperature of 25° C. resulting in a pale-yellow mixture. The mixture was heated at reflux for 4 h. The resulting yellow solution was concentrated in vacuo. Ethyl acetate (50 mL) was added to the concentrated material and the mixture was stirred at 25° C. for 1 h. The solid was filtered to afford crude intermediate 2 (23 g, crude) as white solid. MS (ESI): mass calculated for C₂₂H₂₆N₂O₄. 382.45, m/z found 383.5 [M+H]⁺.

To a solution of 2 (6.1 g. 14.6 mmol) and TEA (4.41, 43.7 mmol) in 100 mL of anhydrous CH₂Cl₂/THF (100 mL) was added trityl chloride (Trt-Cl. 4.47 g, 16.0 mmol). The reaction mixture was stirred at 20° C. for 2 h. The reaction mixture was diluted with water (80 mL, extracted with ethyl acetate (100 mL×2), washed with brine (20 mL) and dried over Na₂SO₄. The combined organic extracts were filtered and concentrated under reduced pressure to afford the crude intermediate 3, which was purified by FCC (eluent: petroleum ether:ethyl acetate=1:0 to 1:2) to afford 3 (7 g, yield: 76.7%) as pale yellow solid. MS (ESI): mass calculated for C₄₁H₄₀N₂O₄, 624.77, m/z found 647.3 [M+Na]+. ¹H NMR (DMSO-d₆, 400 MHz): δ ppm 7.84 (d, J=7.5 Hz, 2H), 7.71 (d, J=7.7 Hz, 1H), 7.66 (d, J=6.8 Hz, 2H), 7.36 (d, J=7.3 Hz, 9H), 7.29-7.20 (m, 8H), 7.17-7.08 (m, 3H), 4.29-4.22 (m, 2H), 4.21-4.11 (m, 1H), 3.97-3.91 (m, 1H), 3.56 (s, 3H), 2.56-2.50 (m, 1H), 1.91 (d, J=6.2 Hz, 2H), 1.55 (m, 2H), 1.46-1.31 (m, 2H), 1.26 (d, J=7.5 Hz, 2H).

A solution of 3 (5.20 g, 8.32 mmol), formaldehyde (20.3 g, 250 mmol) and NaBH₃CN (2.62 g, 41.6 mmol) in methanol (100 mL) was stirred at 25° C. for 16 hours. The mixture was quenched with water (100 mL), extracted with dichloromethane (200 mL×3), the organic layer was dried over anhydrous Na₂SO₄, filtered, and concentrated under reduced pressure. The residue was purified by flash chromatography (FCC, eluent: petroleum ether:ethyl acetate=1:0 to 1:9) to afford 4 (2.7 g, yield: 41.2%) as pale yellow solid. MS (ESI): mass calculated For C₄₂H₄₂N₂O₄, 638.79, m/z found 661.1[M+Na]⁺.

Intermediate 4 (80 g, 125 mmol) was dissolved in HCl/MeOH (800 mL) and stirred at 20° C. for 1 h. The reaction mixture was concentrated under reduced pressure to afford the crude product. Ethyl acetate (100 mL) and petroleum ether (200 mL) were added and the reaction mixture was stirred at 20° C. for 4 h. The solid was filtered to afford intermediate 5 (60 g, crude) as pale yellow solid. MS (ESI): mass calculated for C₂₃H₂₃N₂O₄, 396.48, m/z found 397.1 [M+H]⁺.

To a solution of 5 (120 g, 277 mmol) in CH₂Cl₂ (1200 mL) was added TEA (107 g, 832 mmol) at 0° C. Acetyl chloride (26.1 g, 333 mmol) was added and the reaction mixture was stirred at 20° C. for 2 h. The reaction mixture was diluted with water (300 mL), extracted with CH₂Cl₂ (500 mL×2), washed with brine, and dried over Na₂SO₄. The combined organic extracts were filtered and concentrated under reduced pressure to afford crude intermediate 6, which was purified by FCC (eluent: petroleum ether: ethyl acetate=1:0 to 1:2) to afford 6 (67 g, yield: 38.0%) as pale yellow oil. MS (ESI): mass calculated For C₂₅H₃₀N₂O₅, 438.52, m/z found 439.6 [M+H]⁺.

To a solution 6 (2.6 g, 5.93 mmol) in DCE (50 mL) was added Me3SnOH(1.61 g, 8.90 mmol) and stirred at 20° C. for 16 h. 1 M HCl (5 mL) was added dropwise at 0° C. The mixture was stirred at room temperature for 0.5 h, dried over Na₂SO₄, and filtered. The filtrate was concentrated and the residue was purified by FCC (eluent: CH₂Cl₂: MeOH=1:0 to 95:5) to afford 7 (K(NMeAc), 2.02 g, yield: 80.51%) as pale yellow solid. MS (ESI): mass calculated for C₂₄H₂₈N₂O₅, 424.49, m/z found 425.1 [M+H]+. ¹H NMR (DMSO-d₆, 400 MHz): δ 7.89 (d, J=7.6 Hz, 2H), 7.73 (d, J=7.2 Hz, 2H), 7.62 (m, 1H), 7.46-7.38 (m, 2H), 7.36-7.28 (m, 2H), 4.33-4.16 (m, 3H), 3.89 (s, 1H), 3.22 (m, 2H), 2.93-2.73 (m, 3H), 1.94 (d, J=7.2 Hz, 3H), 1.77-1.55 (m, 2H), 1.55-1.36 (m, 2H), 1.28 (m, 2H).

### h. Synthesis of (S)-2-amino-N-(2-(dimethylamino)-2-oxoethyl)-N-methyl-3-(pyridin-3-yl)propanamide (NH2-3Pva-Sar-CON(Me)2)

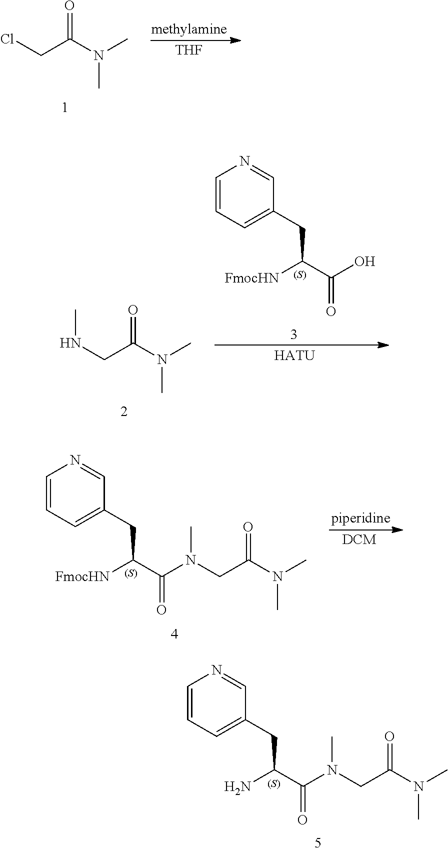

A 100-mL vial was charged with starting material 1 (10 g, 82.3 mmol) and a solution of methylamine (51.1 g, 494 mmol, 30% in ethanol) was added. The reaction mixture was stirred for 16 h at 25° C., after which the mixture was concentrated to give crude intermediate 2. To the crude intermediate, petroleum ether (30 mL) was added and the mixture was stirred at 25° C. for 0.5 h to yield a solid. The resulting solid was filtered to give 2 (10 g, crude) as a light yellow solid. ¹H NMR (DMSO-d₆, 400 MHz): δ ppm 9.09-8.02 (m, 2H), 3.97 (s, 2H), 2.92 (s, 3H), 2.87 (s, 3H), 2.52 (s, 3H).

To a stirred solution of compound 3 (9 g, 23.2 mmol), intermediate 2 (3.23 g, 27.81 mmol), and DIEA (7.03 g, 69.5 mmol) was added in DMF (90 mL) HATU (10.6 g, 27.8 mmol). The reaction mixture was stirred at 25° C. for 2 h then poured into ice water (100 mL), and extracted with ethyl acetate (200 mL×4). The combined organic extracts were washed with brine (100 mL), dried over anhydrous Na₂SO₄, filtered, and concentrated under reduced pressure to afford the crude intermediate 4, which was purified by FCC (eluent: CH₂Cl₂: MeOH=1:0 to 95:5) to afford 4 (11 g, yield: 96.5%) as pale yellow solid. MS (ESI): mass calculated for C₂₈H₃₀N₄O₄, 486.56, m/z found 487.2 [M+H]⁺.

To a solution of 4 (10.5 g, 21.6 mmol) in DCM (400 mL) was added piperidine (5 mL, 50.5 mmol). The reaction mixture was stirred at room temperature for 16 h under a nitrogen atmosphere, and then it was concentrated under vacuum. The residue was purified by FCC (eluent: CH₂Cl₂: MeOH=1:0 to 95:5) to afford crude product 5 (5.5 g, impure) as pale yellow solid. Then crude product was purified by preparative HPLC using a Phenomenex Genimi NX C18 (150*40 mm*5 um) (eluent: 1% to 25% (v/v) water (0.04% NH₃H₂O+10 mM NH₄HCO₃)—MeCN to afford pure product. The pure fractions were collected and lyophilized to dryness to give 5 (NH2-3Pya-Sar-CON(Me)₂, 3.6 g, yield: 62.7%) as a gummy liquid. MS (ESI): mass calculated for C₁₃H₂₀N₄O₂, 264.32, m/z found 265.1 [M+H]+. ¹H NMR (400 MHz, D₂O) δ ppm 8.44-8.22 (m, 2H), 7.76-7.54 (m, 1H), 7.34 (m, 1H), 4.31-4.19 (m, 1H), 4.18-3.96 (m, 2H), 2.95 (m, 3H), 2.92-2.85 (m, 6H), 2.77 (m, 2H).

### i. Synthesis of Substituted Tryptophans

Synthesis of 7-methyl tryptophan. 7-Methyl tryptophan was purchased from a commercial source. Additionally, the compound can be synthesized following one of the methods described below.

Synthesis of 7-ethyl tryptophan. 7-Ethyl tryptophan was synthesized following the method depicted in Scheme 1:

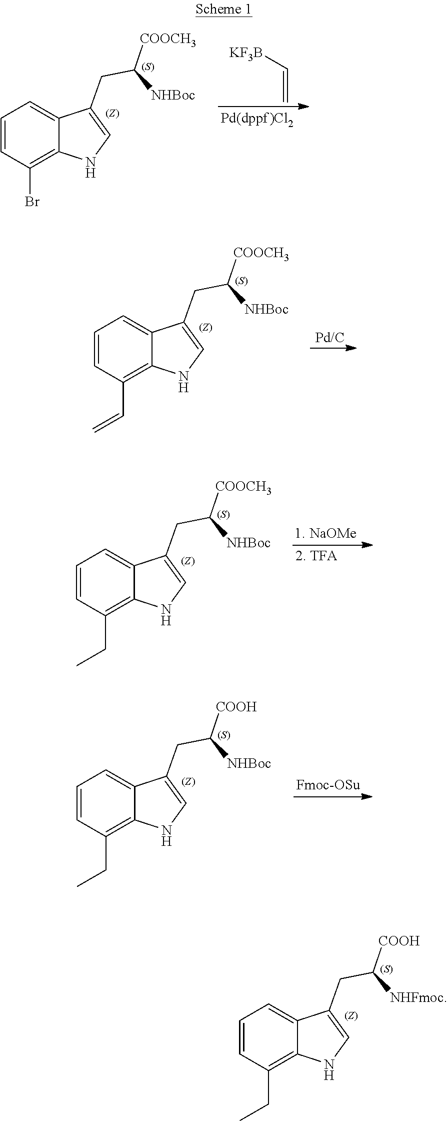

Synthesis of 7-ispropyl tryptophan. 7-Isopropyl tryptophan was synthesized following the method depicted in Scheme 2:

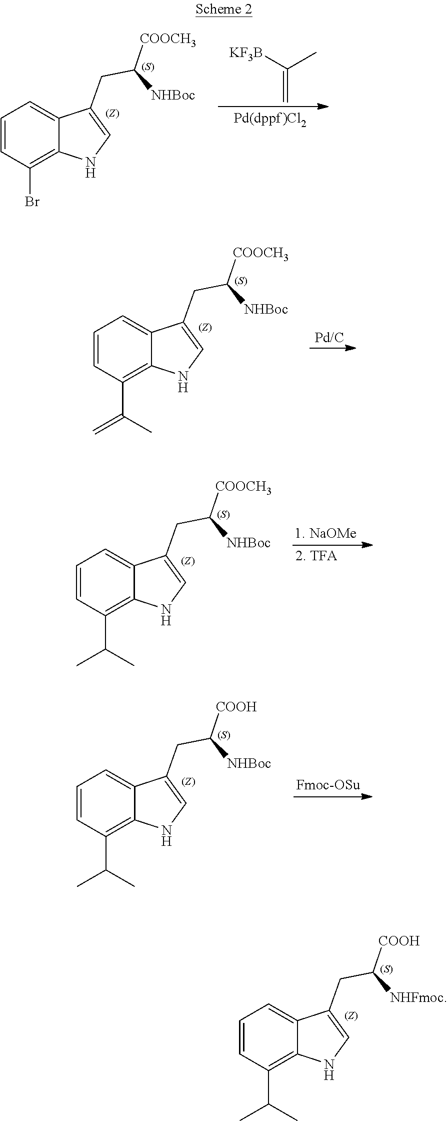

Synthesis of additional 7-substituted tryptophans. Additional 7-substituted tryptophan were or can be synthesized following the method depicted in Scheme 3A:

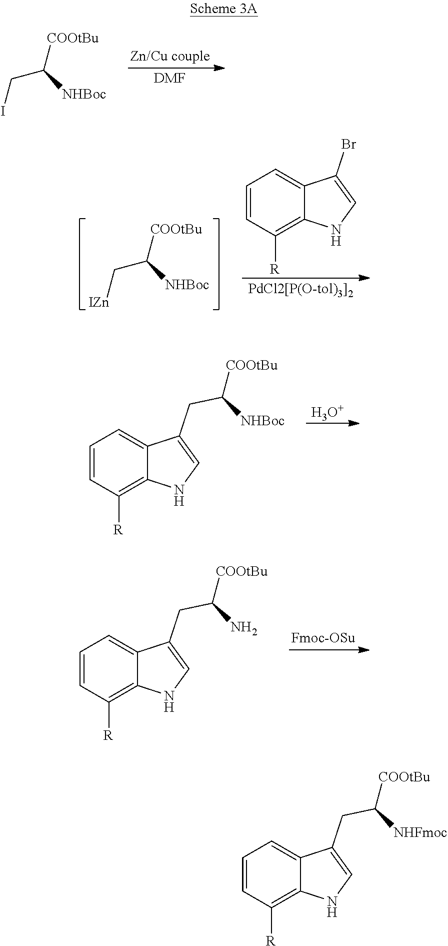

wherein R is cyano, halo, alkyl, haloalkyl, hydroxy, or alkoxy.
Synthesis of 7-aryl substituted tryptophans. 7-Aryl substituted tryptophan were or can be synthesized following the method depicted in Scheme 3B:

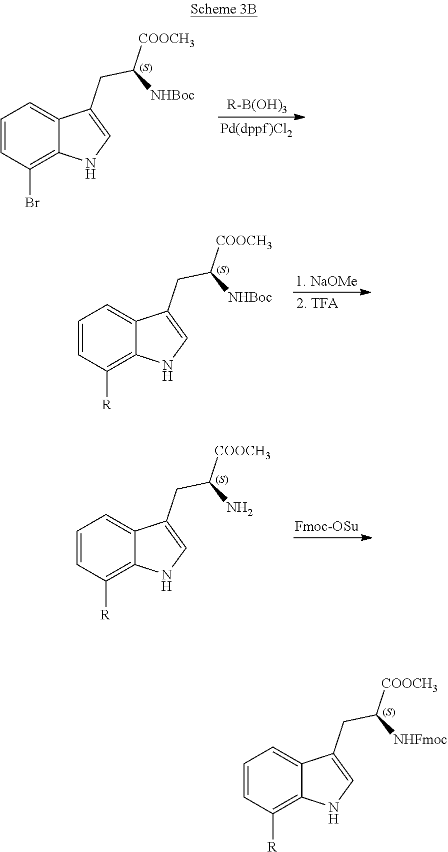

wherein R is aryl, unsubstituted or substituted with halo, alkyl, cyano, haloalkyl, hydroxy, or alkoxy.

Specific representative R groups are selected from phenyl, or 3-Me-phenyl.

Synthesis of 7-phenyl substituted tryptophans. 7-Phenyl substituted tryptophan were or can be synthesized following the method depicted in Scheme 4:

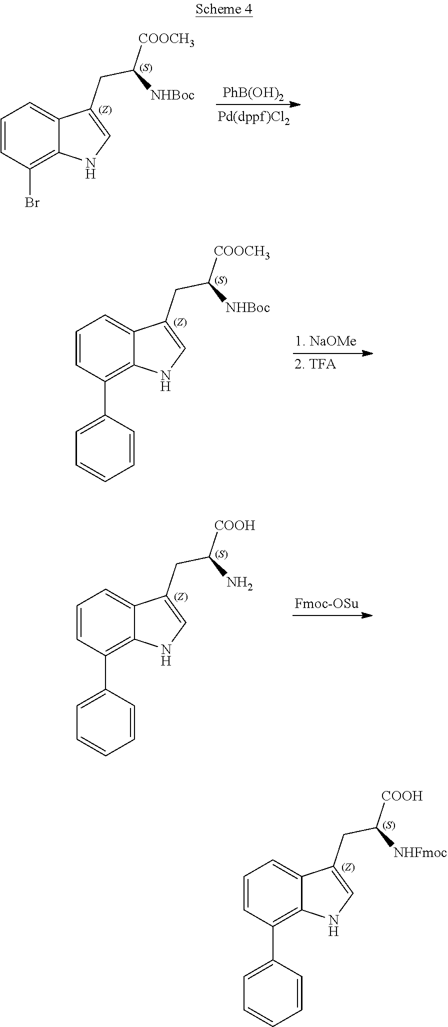

Suzuki Coupling with Aryl Boronic acid. (S)-methyl 3-(7-bromo-1H-indol-3-yl)-2-((tert-butoxycarbonyl)amino)propanoate (4.0 g, 10.0 mmol) in dry toluene (30 mL) was purged for 10 min with nitrogen. K₂CO₃ (2.0 g, 15.0 mmol) in 10 mL of water was added followed by Phenyl boronic acid (1.47 g, 12.0 mmol) and the reaction mixture was purged for 10 min with nitrogen. Pd(dppf)Cl₂.DCM (0.58 g, 0.71 mmol), ethanol (10 mL) and THF (20 mL) were added and the reaction mixture was heated to 100° C. with stirring for 8 hr. The reaction mixture was concentrated under vacuum and the residue was dissolved in DCM (200 mL). The organic layer was washed with water and brine, dried over sodium sulfate and concentrated. The crude product was purified by 60-120-mesh silica gel column chromatography to yield the product (3.6 g, 90%) as foamy solid.

Hydrolysis. To a solution of (S)-methyl 2-((tert-butoxycarbonyl)amino)-3-(7-phenyl-1H-indol-3-yl)propanoate (3.6 g, 9.1 mmol) in THF/MeOH/water (4:1:1) was added lithium hydroxide (1.15 g, 27.3 mmol) and the solution was stirred overnight. The solution was concentrated to remove solvents and diluted with enough water and was acidified with 10% citric acid. The water layer containing product was extracted with ethyl acetate (2×10 mL). The organic layer was washed with water and brine, dried over Na₂SO₄ and concentrated to the desired product (3.3 g, 95%).

Boc Deprotection. To an ice cooled solution of (S)-2-((tert-butoxycarbonyl)amino)-3-(7-phenyl-1H-indol-3-yl)propanoic acid (3.3 g, 8.6 mmol) in dichloromethane (13 mL) was added Trifluoroacetic acid (6.6 mL) and the solution was stirred for 6 h at room temperature. The solution was evaporated to dryness re-dissolved in dichloromethane (10 mL) was treated with HCl/ether to and concentrated. The crude hydrochloride salt was suspended in MTBE (25 mL), stirred for 30 minutes and filtered to get (S)-2-amino-3-(7-phenyl-1H-indol-3-yl)propanoic acid hydrochloride (1.8 g, 66%).

Fmoc protection. To a solution of (S)-2-amino-3-(7-phenyl-1H-indol-3-yl)propanoic acid hydrochloride (1.8 g, 5.7 mmol) in THF/water (45 mL: 13 mL) was added sodium bicarbonate (1.92 g, 22.8 mmol) and then N-(9-Fluorenylmethoxycarbonyloxy)succinimide (1.92 g, 5.7 mmol) in portions. The resulting mixture was stirred overnight and concentrated to remove THF. The residue was diluted with enough water and was acidified with 2N HCl and extracted with ethyl acetate (2×100 mL). The organic layer was washed with water and brine, dried over Na₂SO₄ and concentrated and residue was suspended in 20% MTBE/hexanes to yield the desired product (2.6 g, 92%).

Synthesis of 7-heteroaryl substituted tryptophans. 7-Heteroaryl substituted tryptophan were or can be synthesized following the method depicted in Scheme 5:

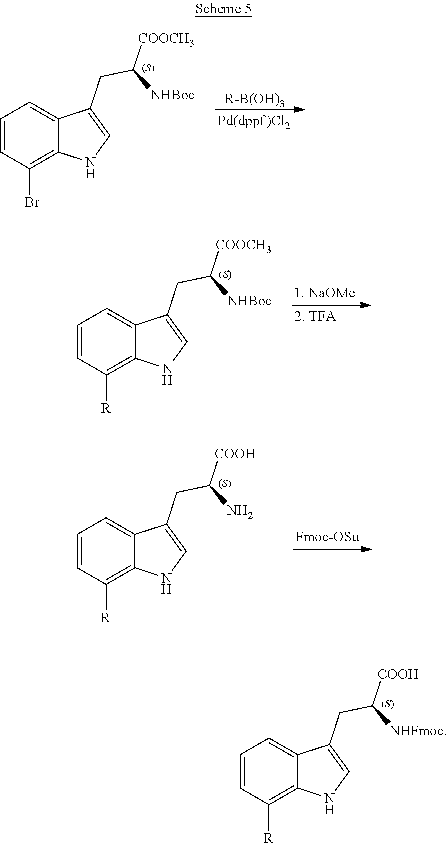

wherein R is heteroaryl, unsubstituted or substituted with halo, halo, alkyl, cyano, haloalkyl, hydroxy, or alkoxy.

Synthesis of 7-heterocycloalkyl substituted tryptophans. 7-Heterocycloalkyl substituted tryptophan were or can be synthesized following the method depicted in Scheme 6:

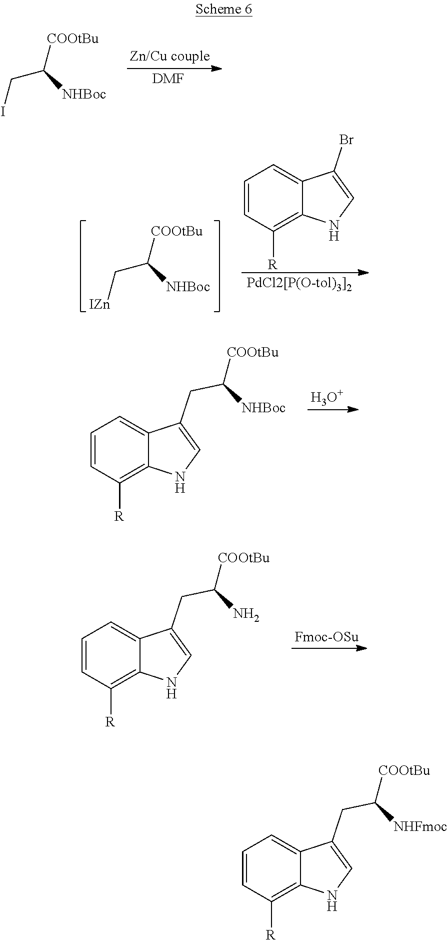

wherein R is heterocycloalkyl, unsubstituted or substituted with alkyl, or halo.

Specific representative R groups are selected from thienyl, pyridyl, piperidinyl, and morpholinyl.

Synthesis of 7-thienyl (thiophenyl) substituted tryptophans. 7-Thienyl (thiophenyl) substituted tryptophan were or can be synthesized following the method depicted in Scheme 7:

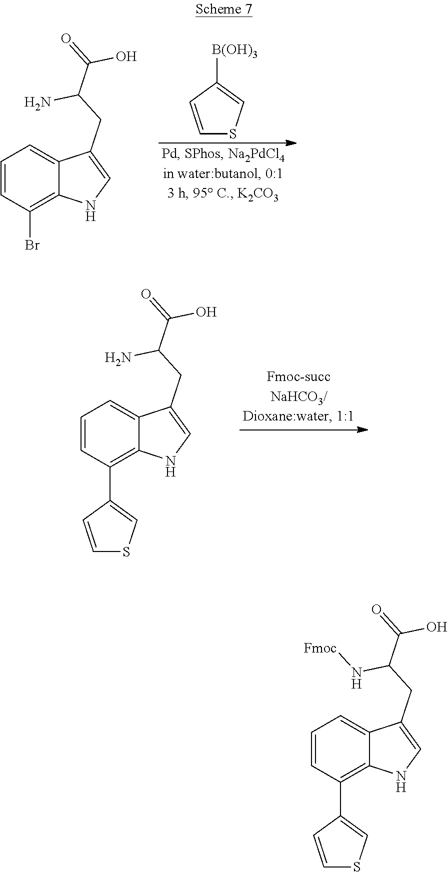

Suzuki-Miyaura cross-coupling reaction was performed using the modified approach described by Frese et al. (ChemCatChem 2016, 8, 1799-1803). Using the Na₂PdCl₄ as a Pd source in combination with the Buchwald ligand SPhos. This system is known to catalyze challenging substrate combinations with excellent results even at low temperatures. In our case the Suzuki-Miyaura cross-coupling reaction of 7 bromoTrp and the boronic acid afforded the wanted product which we subsequently protected using Fmoc-OSu.

L-7-(Thiophen-3-yl)-tryptophan: 7-Bromo-L-tryptophan (0.283 g, 1 mmol), Thiophene-3-boronic acid acid (0.383 g, 3.00 mmol, 3 equiv.) and K₂CO₃ (10 equiv.) were placed in a flask and purged with N₂. Degassed water: 1-butanol (9:1, 30 mL) was added via a syringe, and the reaction was stirred at 95° C. To initiate the reaction SPhos (6.2 mg, 15 mole %) and Na₂Cl₄Pd (15.2 mg, 5 mole %) were transferred to the mixture after previous warming of Pd salt and ligand for 10 min at 40° C.

Upon completion, the aqueous reaction was diluted with H₂O (20 mL) and the solution was acidified to pH 1.0 by dropwise addition of 1 M HCl. Precipitated palladium black was removed by filtration (Whatman, 20 m pore size) and the filtrate was lyophilized. Finally, the resulting crude product was purified by means of preparative reverse-phase high performance liquid chromatography (RP-HPLC) with a C18 column (5 μm, 250×50 mm) with a flow rate of 50 mL/min. Separation was achieved using linear gradients of buffer B in A (Buffer A: Aqueous 0.05% TFA; Buffer B: 0.043% TFA, 90% acetonitrile in water). Analysis was monitored performed using a C18 column (3 μm, 50×2 mm) with a flow rate of 1 mL/min. Fractions containing pure product were then freeze-dried on a lyophilizer. Yield 104 mg (36% yield). MS (ESI) m/z 287.08 [M+H]+(Calcd. For C15H1502NS 287.12).

Fmoc-L-7-(Thiophen-3-yl)-tryptophan: The amino acid, L-7-(Thiophen-3-yl)-tryptophan (31.5 mg, 0.11 mmol) was dissolved in water and sodium bicarbonate (2 eq) with stirring. The resulting solution was cooled to 5° C. and Fmoc-OSu (44.53 mg, 1.05 eq) added slowly as a solution in dioxane. The resulting mixture is stirred at 0° for 1 h and allowed to warm overnight to room temperature. Water was then added and the aqueous layer is extracted 2 times with EtOAc. The organic layer was back extracted twice with saturated sodium bicarbonate solution. The combined aqueous layers are acidified to a pH of 1.0 with 10% HCl, and then extracted 3 times with EtOAc. The combined organic layers are dried (sodium sulfate) and concentrated in vacuo. The resulting residue was be purified by flash chromatography (SiO2) using (toluene, ethyl acteate, (1:1), 1% acetic acid). Yield 50 mg (89% yield). MS (ESI) m/z 509.10 [M+H]+(Calcd. For C15H1502NS 508.59).

### Assembly

The peptides were assembled using standard Fmoc-based solid phase synthesis on various instruments. Generally, tThe peptide sequences were assembled as follows: Resin in each reaction vial was washed twice with DMF followed by treatment with 20% 4-methyl piperidine or 20% piperidine (Fmoc de-protection). The resin was then filtered and washed with DMF and re-treated with 4-methyl piperidine or piperidine. The resin was again washed with DMF followed by addition of amino acid and coupling reagents. After an indicated amount of time of frequent agitations, the resin was filtered and washed with DMF. For a typical peptide of the present invention, double couplings were performed for some amino acids. After completing the coupling reaction, the resin was washed with DMF before proceeding to the next amino acid coupling.

Ring Closing Metathesis to form Olefins

An an example of ring closing metathesis the resin (100 μmol) was washed with 2 ml of DCM (3×1 min) and then with 2 ml of DCE (3×1 min) before being treated with a solution of 2 ml of a 6 mM solution of Grubbs' first-generation catalyst in DCE (4.94 mg ml-1; 20 mol % with regard to the resin substitution). The solution was refluxed overnight (12 h) under nitrogen before being drained. The resin was washed three times with DMF (4 ml each); DCM (4 ml) before being dried and cleaved.

### Cleavage

Following completion of the peptide assembly, the peptide was cleaved from the resin by treatment with cleavage reagent, such as reagent K (82.5% trigluoroacetic acid, 5% water, 5% thioanisole, 5% phenol, 2.5% 1,2-ethanedithiol). The cleavage reagent was able to successfully cleave the peptide from the resin, as well as all remaining side chain protecting groups.

The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered. The quality of linear peptide was then verified using electrospray ionization mass spectrometry (ESI-MS) (Micromass/Waters ZQ) before being purified.

### Disulfide Bond Formation via Oxidation

The peptide containing the free thiol (for example diPen) was assembled on a Rink Amide-MBHA resin following general Fmoc-SPPS procedure. The peptide was cleaved from the resin by treatment with cleavage reagent 90% trifluoroacetic acid, 5% water, 2.5% 1,2-ethanedithiol, 2.5% tri-isopropylsilane). The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered giving the wanted unoxidized peptide crude peptide

Generally, the crude, cleaved peptide with X4 and X9 possessing either Cys, aMeCys, Pen, hCys, (D)Pen, (D)Cys or (D)hCys, was dissolved in 20 ml of water: acetonitrile. Saturated iodine in acetic acid was then added drop wise with stirring until yellow color persisted. The solution was stirred for 15 minutes, and the reaction was monitored with analytic HPLC and LCMS. When the reaction was completed, solid ascorbic acid was added until the solution became clear. The solvent mixture was then purified by first being diluted with water and then loaded onto a reverse phase HPLC machine (an example of conditions include Luna C18 support, 10u, 100 Å, Mobile phase A: water containing 0.1% TFA, mobile phase B: Acetonitrile (ACN) containing 0.1% TFA, gradient began with 5% B, and changed to 50% B over 60 minutes at a flow rate of 15 ml/min). Fractions containing pure product were then freeze-dried on a lyophilyzer.

### Thioether Bond Formation

The peptide containing the free thiol (e.g., Cys) and hSer(OTBDMS) was assembled on a Rink Amide-MBHA resin following general Fmoc-SPPS procedure. Chlorination was carried out by treating the resin with PPh₃ (10 equiv.) and Cl₃CCN (10 equiv.) in DCM for 2 h. The peptide was cleaved from the resin by treatment with cleavage reagent 90% trifluoroacetic acid, 5% water, 2.5% 1,2-ethanedithiol, 2.5% tri-isopropylsilane). The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered giving the wanted uncyclized crude peptide

The crude peptide possessing a free thiol (eg Cys, Pen, aMeCys, hCys, (D)Pen, (D)Cys or (D)hCys and the alkyl halide (hSer(C1)) at either the X4 and X9 position or X9 and X4 position was dissolved in 0.1 M TRIS buffer pH 8.5. Cyclization was allowed to take place overnight at RT. The solvent mixture was then purified by first being diluted two-fold with water and then loaded onto a reverse phase HPLC machine (Luna C18 support, 10u, 100A, Mobile phase A: water containing 0.1% TFA, mobile phase B: Acetonitrile (ACN) containing 0.1% TFA, gradient began with 5% B, and changed to 50% B over 60 minutes at a flow rate of 15 ml/min). Fractions containing pure product were then freeze-dried on a lyophilyzer.

### Purification

Analytical and purification columns and methods vary and are known in the art. For example, analytical reverse-phase, high performance liquid chromatography (HPLC) was performed on a Gemini C18 column (4.6 mm×250 mm) (Phenomenex). Semi-Preparative reverse phase HPLC was performed on a Gemini 10 m C18 column (22 mm×250 mm) (Phenomenex) or Jupiter® 10 m, 300 angstrom (A) C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative).

### Example 1B. ac-Pen(1:3)-E-T-Trp 7Me-Lys Ac-Pen(1:3)-Phe 4 2ae-Nal-THP-Lys Ac—N—H-Sar-am (Intermediate Peptide)

The TFA (Trifluoroacetic acid) salt of the Intermediate Peptide was synthesized on a 0.1 mmol scale. Upon completion, 60 mg of ˜95% pure intermediate peptide was isolated as a white powder, representing an overall yield of ˜30%.

The Intermediate Peptide was synthesized using the Merrifield solid phase synthesis techniques on Protein Technology's Symphony multiple channel synthesizer and constructed on Rink Amide MBHA (100-200 mesh, 0.8 mmol/g) resin using standard Fmoc protection synthesis conditions. The constructed peptide was isolated from the resin and protecting groups by cleavage with strong acid followed by precipitation. The crude leaner peptide was then cyclized and purified by reverse-phase, high performance liquid chromatography (RP-HPLC). Lyophilization of pure fractions gave the final product of intermediate peptide 2.

Swell Resin: 125 mg of Rink Amide MBHA resin (0.1 mmol, 0.8 mmol/g loading) was transferred to a 25 mL reaction vessel (for Symphony peptide synthesizer). The resin was swelled with 3.75 mL of DMF (3×10 min)

Step 1: Coupling of Fmoc-Sar-OH (Fmoc-N-methylglycine): Deprotection of the Fmoc group was accomplished by two treatments with 2.5 ml of 20% piperidine in DMF twice to the swollen Rink Amide resin for 5 and 10 min respectively. After deprotection the resin was washed with 3.75 mL of DMF (3×0.1 min) and followed by addition of 2.5 mL of amino acid Fmoc-Sar-OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 2: Coupling of Fmoc-His(Trt)-OH: The resin was washed with 3.75 mL of DMF (3×0.1 min) and the Fmoc group was removed from the N-terminus of the Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-His(Trt)-OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 3: Coupling of Fmoc-Asn(Trt)-OH: The resin was washed with 3.75 mL of DMF (3×0.1 min) and the Fmoc group was removed from the N-terminus of the His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-His(Trt)-OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 4: Coupling of Fmoc-Lys(Ac)—OH: The resin was washed with 3.75 mL of DMF (3×0.1 min) and the Fmoc group was removed from the N-terminus of the Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-Lys(Ac)—OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 5: Coupling of Fmoc-THP-OH (Fmoc-4-amino-tetrahydropyran-4-carboxylic acid): The resin was washed with 3.75 mL of DMF (3×0.1 min) and the Fmoc group was removed from the N-terminus of the Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-THP-OH in DMF (100 mM) and 1.25 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 6: Coupling of Fmoc-2Nal-OH (Fmoc-3-(2-naphthyl)-L-alanine): The resin was washed with 3.75 mL of DMF (3×0.1 min) and the Fmoc group was removed from the N-terminus of the THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-2Nal-OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 7: Coupling of Fmoc-Phe_4_2ae-OH (Fmoc-4-[2-(Boc-amino)ethoxy]-L-phenylalanine): The resin was washed with 3.75 mL of DMF (3×0.1 min) and the Fmoc group was removed from the N-terminus of the 2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-Phe_4_2ae-OH in DMF (100 mM) and 1.25 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 8: Coupling of Fmoc-L-Pen(Trt)-OH (Fmoc-S-trityl-L-penicillamine): The resin was washed with 3.75 mL of DMF (3×0.1 min) and the Fmoc group was removed from the N-terminus of the Phe_4_ae-2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-L-Pen(Trt)-OH in DMF (100 mM) and 1.25 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 9: Coupling of Fmoc-Lys(Ac)—OH: The resin was washed with 3.75 mL of DMF (3×0.1 min) and the Fmoc group was removed from the N-terminus of the Pen-Phe_4_ae-2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-Lys(Ac)—OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 10: Coupling of Fmoc-Trp_7Me-OH: The resin was washed with 3.75 mL of DMF (3×0.1 min) and the Fmoc group was removed from the N-terminus of the Lys(Ac)-Pen-Phe_4_ae-2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-Trp_7Me-OH in DMF (100 mM) and 1.25 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 11: Coupling of Fmoc-Thr(tBu)-OH: The resin was washed with 3.75 mL of DMF (3×0.1 min) and the Fmoc group was removed from the N-terminus of the Trp_7Me-Lys(Ac)-Pen-Phe_4_ae-2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-Thr(tBu)-OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 12: Coupling of Fmoc-Glu(OtBu)-OH: The resin was washed with 3.75 mL of DMF (3×0.1 min) and the Fmoc group was removed from the N-terminus of the Thr-Trp_7Me-Lys(Ac)-Pen-Phe_4_ae-2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-Glu(OtBu)-OH in DMF (200 mM) and 2.5 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 13: Coupling of Fmoc-L-Pen(Trt)-OH (Fmoc-S-trityl-L-penicillamine): The resin was washed with 3.75 mL of DMF (3×0.1 min) and the Fmoc group was removed from the N-terminus of the Glu-Thr-Trp_7Me-Lys(Ac)-Pen-Phe_4_ae-2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of amino acid Fmoc-L-Pen(Trt)-OH in DMF (100 mM) and 1.25 mL of coupling reagent HBTU-DIEA mixture in DMF (200 and 220 mM). The coupling reaction was mixed for 1 hr, filtered and repeated once (double couplings). After completing the coupling reaction, the resin was washed with 6.25 mL of DMF (3×0.1 min) prior to starting the next deprotection/coupling cycle.

Step 14: Acetyl Capping: The resin was washed with 3.75 mL of DMF (3×0.1 min) and the Fmoc group was removed from the N-terminus of the Pen-Glu-Thr-Trp_7Me-Lys(Ac)-Pen-Phe_4_ae-2Nal-THP-Lys(Ac)-Asn-His-Sar-Rink Amide resin by two treatments with 2.5 ml of 20% piperidine in DMF for 5 and 10 min respectively. After deprotection the resin was washed with 3.75 mL of DMF (3×0.1 min) and followed by addition of 2.5 mL of 20% acidic anhydride in DMF and 2.5 mL of 10% DIEA in DMF. The acetyl reaction was mixed for 1 hr, filtered and repeated once (double couplings). After completing the acetylation, the resin was washed with 6.25 mL of DMF (6×0.1 min) and 6.25 mL of DCM (6×0.1 min), followed by drying under the nitrogen for 20 min prior to cleavage with TFA.

Step 15: TFA Cleavage and Ether Precipitation: Following the completion of the peptide assembly, the dried resin was transferred into 20 mL glass vial. To this 10 mL of the TFA cleavage cocktail (90/5/2.5/2.5 of TFA/water/Tips/DODT) was added and stirred at room temperature for 2 hrs. The cleavage reagent was able to cleave the peptide from the resin, as well as all remaining side chain protecting groups. After that, the majority of TFA was blown off under the nitrogen and 20 mL of cold diethyl ether was then added to the rest of the peptide cleavage mixture forming a white precipitate. The ether mixture was centrifuged at 3000 rpm for 3 min at 4° C. and the ether layer (containing side chain protecting groups) was decanted to the waste and 2 more ether washes (20 mL each) of the precipitate (cleaved peptide) were performed. The crude linear peptide (pellet) was dissolved in 40 mL of acetonitrile:water (1:1) and filtered through 0.45 μm RC membrane to remove the resin.

Step 16: Disulfide Bond Formation via Oxidation: The crude linear peptide was oxidized without the purification. After the cleavage step, crude linear peptide in 40 mL of 50% acetonitrile in water was diluted to 100 mL with water to make a final organic solvent content of 20% acetonitrile in water. To this a saturated solution of iodine in methanol was added dropwise while stirring until the yellow color remained and did not fade away. The slightly colored solution was stirred for extra 5 min prior to quenching the excess iodine by adding a pinch of solid ascorbic acid until the solution became clear.

Step 17: RP-HPLC Purification of Monocyclic Peptide (Disulfide Bond): The purification was carried out using RP-HPLC. A semi-preparative column Gemini 5 μm C18 column (21.2 mm×250 mm) (Phenomenex©) was equilibrated at the flow rate of 20 mL/min with 100% mobile phase A (MPA=0.1% TFA in water). The 100 mL of quenched oxidized peptide was loaded onto the equilibrated column directly at 20 mL/min, and washed with 20% mobile phase B (MPB=0.1% TFA in acetonitrile) for 5 min. The Separation was achieved using linear gradient of 20-50% MPB over 30 min at 20 mL/min. The desired oxidized peptide eluted at ˜30% MPB. Pure fractions were combined and lyophilized to give 60 mg of purified oxidized peptide in the format of TFA salt, with yield of 30%.

Step 18: Characterization: After lyophilization gave a white powder with a purity of >95% by analytical HPLC. Low resolution Liquid chromatography-mass spectrometry (LC-MS) gave triply charged ion [M+3H]³⁺ of 648.7 and doubly charged ion [M+2H]²⁺ of 972.4. The experimental mass agrees with the theoretical molecular weight of 1943.27 Da.

### Example 1C. ac-Pen(1:3)-E(2:3)-T-Trp 7Me-Lys Ac-Pen(1:3)-Phe 4 2ae(2:3)-Nal-THP-Lys Ac—N—H-Sar-am

The TFA (Trifluoroacetic acid) salt of bicyclic title compound was synthesized on a 0.01 mmol scale using purified monocyclic peptide precursor (step 1-17) as described previously, followed by the lactam bond formation (between residue Glu and Phe_4_2ae) and purified by RP-HPLC. Upon completion, 10 mg of ˜95% pure title compound was isolated as a white powder, representing a yield of 50% for the step of lactam bond formation and total yield of 15%.

Step 18: Lactam Bond Formation: 20 mg of purified oxidized intermediate peptide (˜0.01 mmol) was dissolved in 10 mL of N N-Dimethylformamide (DMF). To this (benzotriazol-1-yl-oxytripyrrolidinophosphonium hexafluorophosphate (PyBOP) (0.04 mmol, 4 equivalents) was added followed by N,N-Diisopropylethylamine (DIEA) (0.05 mmol, 5 equivalents). The mixture was stirred at room temperature and the reaction was monitored by analytical HPLC. The reaction was completed within 30 min and the mixture was diluted with 20% acetonitrile in water to 100 mL with the final content of DMF <10% prior to loading onto the HPLC for purification.

Step 19: RP-HPLC Purification of Bicyclic Peptide (Disulfide Bond and Lactam Bond): The 2^{nd} purification was carried out using the same procedure as described previously in Step 17. The desired bicyclic peptide eluted later then monocyclic peptide at ˜35% MPB. Pure fractions were combined and lyophilized to give 10 mg of purified bicyclic peptide in the format of TFA salt, with yield of 50% for the step of lactam bond formation and total yield of 15%.

Step 20: Characterization: After lyophilization, the title compound gave a white powder with a purity of >95% by analytical HPLC. Low resolution Liquid chromatography-mass spectrometry (LC-MS) gave triply charged ion [M+3H]³⁺ of 642.5 and doubly charged ion [M+2H]²⁺ of 963.5. The experimental mass agrees with the theoretical molecular weight of 1925.26 Da.

### Example 1D. ac-Pen(1:3)-Dap(2:3)-T-Trp 7Me-Lvs Ac-Pen(1:3)-Phe 4 2ae(3:3)-Nal-THP-Lys Ac—N—H-Sar-am-PEG4DA(x,2:1,3:2)

The TFA (Trifluoroacetic acid) salt of bicyclic title compound was synthesized on a 0.01 mmol scale using its corresponding purified monocyclic (disulfide bond) peptide precursor and the 2^{nd} cyclization was carried out using pre-activated diacid linker conjugate onto primary amine on the side chain of residue Dap(2:3) and Phe_4_2ae, followed by the purification using RP-HPLC. Upon completion, 10 mg of ˜95% pure title compound was isolated as a white powder, representing a yield of 50% for the step of 2^{nd} cyclisation and total yield of 15%.

Preparation of Monocyclic Precursor: the purified monocyclic precursor (disulfide bond) was prepared similarly as intermediate as described previously (step 1-17), except for step 12, using amino acid of Fmoc-L-Dap(Boc)-OH (Na-Fmoc-N$-Boc-L-2,3-diaminopropionic acid) instead of Fmoc-Glu(OtBu)-OH.

Step 18: Diacid Linker Activation: Bis-PEG4-acid (PEG4DA) (294 mg, 1 mmol), N-Hydroxysuccinimide (NHS) (2.2 mmol, 2.2 equivalents) and N,N′-Dicyclohexylcarbodiimide (DCC) (2.2 mmol, 2.2 equivalents) were dissolved in 10 mL N-Methyl-2-pyrrolidone (NMP). The mixture was stirred at room temperature to completely dissolve the solid starting materials. Precipitation appeared within 10 min and the reaction mixture was further stirred at room temperature overnight and was then filtered to remove the precipitated dicyclohexylurea (DCU). The activated linker was kept in a closed glass vial at 4° C. prior to use for 2^{nd} cyclization. The nominal concentration of the pre-activated linker was approximately 0.1 M.

Step 19: Bicyclic Formation via Pre-activated Diacid Linker (PEG4DA-NHS): 20 mg of purified monocyclic precursor (˜0.01 mmol) was dissolved in 10 mL of N N-Dimethylformamide (DMF). To this pre-activated diacid linker (PEG4DA-NHS) (0.1 M in NMP, 0.01 mmol, 1 equivalent) and N,N-Diisopropylethylamine (DIEA) (0.1 mmol, 10 equivalents) were added stepwise over the 10 min. The mixture was stirred at room temperature and the reaction was monitored by analytical HPLC. Excess equivalent of PEG4DA-NHS may be required to drive the reaction to completion. The reaction was completed after 1 hr and the mixture was diluted with 20% acetonitrile in water to 100 mL with the final content of DMF <10% prior to loading onto the HPLC for purification.

Step 20: RP-HPLC Purification of Bicyclic Peptide: The 2^{nd} purification was carried out using the same procedure as described previously in Step 17. The desired bicyclic peptide eluted later then monocyclic peptide at ˜35% MPB. Pure fractions were combined and lyophilized to give 10 mg of purified bicyclic peptide in the format of TFA salt, with yield of 50% for the step of lactam bond formation and total yield of 15%.

Step 21: Characterization: After lyophilization, the title compound gave a white powder with a purity of >95% by analytical HPLC. Low resolution Liquid chromatography-mass spectrometry (LC-MS) gave triply charged ion [M+3H]³⁺ of 720.1 and doubly charged ion [M+2H]²⁺ of 1080.1. The experimental mass agrees with the theoretical molecular weight of 2158.52 Da.

### Example 1E. Ac-[Pen]*-E**-T-[W(7-Me)]-[Lys(Ac)]-[Pen]*-Phe[4-(2-aminoethoxy)]**-[2-Nal]-[THP]-E-N-[3-Pal]-Sarc-NH₂ (*Pen-Pen form disulfide bond) (**Side chain of Glu and Phe[4-(2-aminoethoxy) form lactam bond)

The synthesis of the title compound was prepared using FMOC solid phase peptide synthesis techniques.

The title compound was constructed on Rink Amide MBHA resin using standard FMOC protection synthesis conditions reported in the literature. The constructed peptide was isolated from the resin and protecting groups by cleavage with strong acid followed by precipitation. Oxidation to form the disulfide bond was performed followed by purification by RPHPLC and counterion exchange. Lyophilization of pure fractions gives the final product.

Swell Resin: 10 g of Rink Amide MBHA solid phase resin (0.66 mmol/g loading) is transferred to a 250 ml peptide vessel with filter frit, ground glass joint and vacuum side arm. The resin was washed 3× with DMF.

Step 1: Coupling of FMOC-Sarc-OH: Deprotection of the resin bound FMOC group was realized by adding 2 resin-bed volumes of 20% 4-methyl-piperidine in DMF to the swollen resin and shaking for 3-5 min prior to draining and adding a second, 2-resin-bed volume of the 4-methyl piperidine solution and shaking for an additional 20-30 min. After deprotection the resin was washed 3×DMF with shaking. FMOC-Sarc-OH (3 eq, 6.2 g) was dissolved in 100 ml DMF along with Oxyma (4.5 eq, 4.22 g). Preactivation of the acid was accomplished by addition of DIC (3.9 eq, 4 ml) with shaking for 15 min prior to addition to the deprotected resin. An additional aliquot of DIC (2.6 eq, 2.65 ml) was then added after ˜ 15 min of coupling. The progress of the coupling reaction was monitored by the colorimetric Kaiser test. Once the reaction was judged complete the resin was washed 3×DMF with shaking prior to starting the next deprotection/coupling cycle.

Step 2: Coupling of FMOC-3Pal-OH: FMOC deprotection was again accomplished by adding two sequential, 2-resin-bed volumes of 20% 4-methyl-piperidine in DMF, one times 3-5 minutes and one times 20-30 minutes, draining in between treatments. The resin was then washed 3 times prior to coupling with protected 3-pyridyl alanine (3Pal). FMOC-3Pal-OH (3 eq, 7.8 g) was dissolved in DMF along with Oxyma (4.5 eq, 4.22 g). Preactivation with DIC (3.9 eq, 4 ml) for 15 minutes was done prior to addition to the Sarc-Amide resin. After 15 minutes, an additional aliquot of DIC (2.6 eq, 2.65 ml) was added to the reaction. Once the reaction was complete as determined by the Kaiser test, the resin was again washed 3× with DMF prior to starting the next deprotection/coupling cycle.

Step 3: Coupling of FMOC-Asn(Trt)-OH: The FMOC was removed from the N-terminus of the resin bound 3Pal and washed as previously described. FMOC-Asn(Trt)-OH (2 eq, 8 g) was dissolved in 100 ml of DMF along with Oxyma (3 eq, 2.81 g). DIC (2.6 eq, 2.65 ml) was added for preactivation of the acid for ˜15 minutes prior to addition to the 3Pal-Sarc-Amide resin. After ˜15 minutes, an additional aliquot of DIC (1.4 eq, 1.43 ml) was added to the reaction. Once the reaction was complete as determined by the Kaiser test, the resin was washed 3× with DMF prior to starting the next deprotection/coupling cycle.

Step 4: Coupling of FMOC-Lys(Ac)—OH: The FMOC was removed from the N-terminus of the resin bound peptide and the resin washed as previously described. FMOC-Lys(Ac)—OH (2 eq, 5.4 g) was dissolved in 100 ml of DMF along with Oxyma (3 eq, 2.81 g). DIC (2.6 eq, 2.65 ml) was added for preactivation of the acid ˜15 minutes prior to addition to the Asn(Trt)-3Pal-Sarc-Amide resin. After ˜15 minutes, an additional aliquot of DIC (1.4 eq, 1.43 ml) was added to the reaction. Once the reaction was complete as determined by the Kaiser test, the resin was again washed 3× with DMF prior to starting the next deprotection/coupling cycle.

Step 5: Coupling of FMOC-THP-OH: The FMOC was removed from the N-terminus of the resin bound peptide and the resin was washed as previously described. FMOC-THP-OH (3 eq, 7.36 g) was dissolved in 100 ml of DMF along with Oxyma (4.5 eq, 4.22 g). DIC (3.9 eq, 4 ml) was added for preactivation of the acid ˜15 minutes prior to addition to the Lys(Ac)-Asn(Trt)-3Pal-Sarc-Amide resin. After ˜15 minutes, an additional aliquot of DIC (2.6 eq, 2.65 ml) was added to the reaction. Once the reaction is complete as determined by the Kaiser test the resin was washed 3× with DMF prior to starting the next deprotection/coupling cycle.

Step 6: Coupling of FMOC-L-Ala(2-Naphthyl)-OH (Nal): The FMOC was removed from the N-terminus of the resin bound peptide and the resin washed as previously described. FMOC-L-Ala(2-Naphthyl)-OH (3 eq, 8.66 g) was dissolved in 100 ml of DMF along with Oxyma (4.5 eq, 4.22 g). DIC (3.9 eq, 4 ml) was added for preactivation of the acid ˜15 minutes prior to addition to the THP-Lys(Ac)-Asn(Trt)-3Pal-Sarc-Amide resin. After ˜15 minutes, an additional aliquot of DIC (2.6 eq, 2.65 ml) was added. Once the reaction was complete as determined by the Kaiser test the resin was again washed 3× with DMF prior to starting the next deprotection/coupling cycle.

Step 7: Coupling of FMOC-4-[2-(Boc-amino-ethoxy)]-L-Phenylalanine (FMOC-AEF): The FMOC was removed from the N-terminus of the resin bound peptide and the resin washed as previously described. FMOC-4-[2-(Boc-amino-ethoxy)]-L-Phenylalanine (3 eq, 10.8 g) was dissolved in 100 ml of DMF along with Oxyma (4.5 eq, 4.22 g). DIC (3.9 eq, 4 ml) was added for preactivation of the acid ˜15 minutes prior to addition to the Nal-THP-Lys(Ac)-Asn(Trt)-3Pal-Sarc-Amide resin. After ˜15 minutes, an additional aliquot of DIC (2.6 eq, 2.65 ml) was added to the reaction. Once the reaction was complete as determined by the Kaiser test the resin is washed 3× with DMF prior to starting the next deprotection/coupling cycle.

Step 8: Coupling of FMOC-Pen(Trt)-OH: The FMOC was removed from the N-terminus of the resin bound peptide and the resin washed as previously described. FMOC-Pen(Trt)-OH (3 eq, 12.14 g) was dissolved in 100 ml of DMF along with Oxyma (4.5 eq, 4.22 g). DIC (3.9 eq, 4 ml) was added for preactivation of the acid ˜15 minutes prior to addition to the AEF-Nal-THP-Lys(Ac)-Asn(Trt)-3Pal-Sarc-Amide resin. After ˜15 minutes, an additional aliquot of DIC (2.6 eq, 2.65 ml) was added to the reaction. Once the reaction is complete as determined by the Kaiser test, the resin was again washed 3× with DMF prior to starting the next deprotection/coupling cycle.

Step 9: Coupling of FMOC-Lys(Ac)—OH: The FMOC was removed from the N-terminus of the resin bound peptide and the resin washed as previously described. FMOC-Lys(Ac)—OH (2 eq, 5.4 g) was dissolved in 100 ml of DMF along with Oxyma (3 eq, 2.81 g). DIC (2.6 eq, 2.65 ml) was added for preactivation of the acid ˜15 minutes prior to addition to the Pen(Trt)-AEF-Nal-THP-Lys(Ac)-Asn(Trt)-3Pal-Sarc-Amide resin. After ˜15 minutes, an additional aliquot of DIC (1.4 eq, 1.43 ml) was added to the reaction. Once the reaction was complete as determined by the Kaiser test, the resin was again washed 3× with DMF prior to starting the next deprotection/coupling cycle.

Step 10: Coupling of FMOC-7-Me-Trp-OH: The FMOC was removed from the N-terminus of the resin bound peptide and the resin washed as previously described. FMOC-7-Me-Trp-OH (2 eq, 5.81 g) was dissolved in 100 ml of DMF along with Oxyma (3 eq, 2.81 g). DIC (2.6 eq, 2.65 ml) was added for preactivation of the acid ˜15 minutes prior to addition to the Lys(Ac)-Pen(Trt)-AEF-Nal-THP-Lys(Ac)-Asn(Trt)-3Pal-Sarc-Amide resin. After ˜15 minutes, an additional aliquot of DIC (1.4 eq, 1.43 ml) was added to the reaction. Once the reaction was complete as determined by the Kaiser test, the resin was again washed 3× with DMF prior to starting the next deprotection/coupling cycle.

Step 11: Coupling of FMOC-Thr(tBu)-OH: The FMOC was removed from the N-terminus of the resin bound peptide and the resin washed as previously described. FMOC-Thr(tBu)-OH (4 eq, 10.5 g) was dissolved in 100 ml of DMF along with Oxyma (6 eq, 5.62 g). DIC (5.2 eq, 5.3 ml) was added for preactivation of the acid A15 minutes prior to addition to the 7MeTrp-Lys(Ac)-Pen(Trt)-AEF-Nal-THP-Lys(Ac)-Asn(Trt)-3Pal-Sarc-Amide resin. After ˜15 minutes, an additional aliquot of DIC (2.6 eq, 2.65 ml) was added to the reaction. Once the reaction was complete as determined by the Kaiser test, the resin was again washed 3× with DMF prior to starting the next deprotection/coupling cycle.

Step 12: Coupling of FMOC-Glu(OtBu)-OH: The FMOC was removed from the N-terminus of the resin bound Asparigine and the resin washed with DMF as previously described. FMOC-Glu(OtBu)-OH (2 eq, 5.91 g) was dissolved in 100 ml of DMF along with Oxyma (3 eq, 2.81 g). DIC (2.6 eq, 2.65 ml) was added for preactivation of the acid ˜15 minutes prior to addition to the Thr(tBu)-7MeTrp-Lys(Ac)-Pen(Trt)-AEF-Nal-THP-Lys(Ac)-Asn(Trt)-3Pal-Sarc-Amide resin. After ˜15 minutes, an additional aliquot of DIC (1.4 eq, 1.43 ml) was added to the reaction. Once the reaction was complete as determined by the Kaiser test the resin was washed 3× with DMF prior to starting the next deprotection/coupling cycle.

Step 13: Coupling of FMOC-Pen(Trt)-OH: The FMOC was removed from the N-terminus of the resin bound peptide and the resin washed as previously described. FMOC-Pen(Trt)-OH (2 eq, 8.1 g) was dissolved in 100 ml of DMF along with Oxyma (3 eq, 2.81 g). DIC (2.6 eq, 2.65 ml) was added for preactivation of the acid ˜15 minutes prior to addition to the Glu(OtBu)-Thr(tBu)-7MeTrp-Lys(Ac)-Pen(Trt)-AEF-Nal-THP-Lys(Ac)-Asn(Trt)-3Pal-Sarc-Amide resin. After ˜15 minutes, an additional aliquot of DIC (2.6 eq, 2.65 ml) was added to the reaction. Once the reaction was complete as determined by the Kaiser test, the resin was again washed 3× with DMF prior to the final deprotection and acetic acid capping of the constructed peptide.

Step 14: Acetyl Capping: The FMOC was removed from the N-terminus of the resin bound peptide and the resin washed as previously described. 150 ml of Capping Reagent A (THF/Acetic anhydride/Pyridine, 80:10:10) was added to the constructed Pen(Trt)-Glu(OtBu)-Thr(tBu)-7MeTrp-Lys(Ac)-Pen(Trt)-AEF-Nal-THP-Lys(Ac)-Asn(Trt)-3Pal-Sarc-Amide resin and shaken for 30 min. The resin was washed 3× with DMF followed by 5× with DCM. The resin was divided into 5-50 ml centrifuge tubes and placed under vacuum for 1.5 hrs prior to cleavage with TFA.

Step 15: TFA Cleavage and Ether precipitation: 200 ml of the TFA cleavage cocktail (90/5/2.5/2.5 TFA/water/Tips/DODT) was prepared. 40 ml of the cleavage cocktail was added to each of the 5 tubes containing the protected resin bound peptide and shaken for two hours. The spent resin was filtered away and the filtrate divided evenly into 18-50 ml centrifuge tubes for precipitation. Cold diethyl ether was added to each forming a white precipitate that was then centrifuged. The ether was decanted to waste and 2 more ether washes of the precipitate are performed. The resulting white precipitate cake was dried overnight in the hood to give the crude reduced peptide.

Step 16: Disulfide Oxidation: The crude peptide was oxidized and purified in four 1 L batches. ˜2.5 g of crude peptide was dissolved in 1 L 20% ACN/water. With stirring, a saturated solution of iodine in acetic acid/methanol was added dropwise to the 1 L peptide solution until the yellow/brown color of the I₂ remains and does not fade away. The light yellow solution was allowed to sit for 5 min prior to quenching the excess I₂ with a pinch of ascorbic acid.

Step 17: RP-HPLC purification: The RP-HPLC purification was performed immediately following each 12 oxidation. A preparative purification column (Phenomenex, Luna, C18(2), 100A, 250×50 mm) was equilibrated at 70 ml/min with 20% MPB in MPA (MPA=0.1% TFA/water, MPB=0.1% TFA in ACN). The 1 L of quenched oxidized peptide was loaded onto the equilibrated column at 70 ml/min. After the solvent front elutes, a gradient of 25-45% MPB at 70 ml/min was run over 60 min. The desired material was isolated in fractions and each are analyzed by analytical RPHPLC. Pure fractions are combined from all four purifications and lyophilized to give purified TFA salt ready for bicyclization via lactam formation.

Step 18: Lactam formation to give bicycle: The purified Pen-Pen disulfide monocyclic peptide (800 mg) was dissolved in 150 ml of 50/50 DMF/DCM (˜ 5 mg/ml). To the stirring peptide was added diisopropylethylamine (˜5 eq, 360 ul), followed by PyBop (˜4 eq, 864 mg). The reaction was monitored by RP-HPLC. Once all monocyclic starting material was converted to the bicyclic form, the solution was neutralized and diluted to 1 L with 10% Acetonitrile in water. The diluted solution was ready for RP-HPLC purification.

Step 19: RP-HPLC purification: The RP-HPLC purification was performed immediately following lactam formation and dilution. A preparative purification column (Phenomenex, Luna, C18(2), 100A, 250×50 mm) was equilibrated at 70 ml/min with 20% MPB in MPA (MPA=0.1% TFA/water, MPB=0.1% TFA in ACN). The 1 L of the neutralized bicyclic peptide was loaded onto the equilibrated column at 70 ml/min. After the solvent front elutes, a gradient of 25-45% MPB at 70 ml/min was run over 60 min. The desired material was isolated in fractions and each are analyzed by analytical RPHPLC. Pure fractions are combined from all four purifications and lyophilized to give purified TFA salt ready for counterion exchange.

Step 20: Counterion Exchange to Acetate: The same preparative RP-HPLC column was equilibrated with 5% MPB in MPA at 70 ml/min (MPA=0.3% AcOH in Water, MPB=0.3% AcOH in ACN, MPC=0.5M NH4OAc in Water.) The purified peptide TFA salt was dissolved in 50/50 ACN/water and diluted to 15% ACN. The solution was loaded onto the equilibrated column at 70 ml/min and the solvent front was eluted. The captured peptide was washed with 5% MPB in MPA for 5 min. The captured peptide was then washed with 5% MPB in MPC for 40 min at 70 ml/min to exchange the counterions to Acetate. The captured peptide was washed with 5% MPB in MPA at 70 ml/min for 10 min to clear all NH4OAc from the system. Finally, the peptide was eluted with a gradient of 5-70% MPB in MPA over 60 minutes and collected in fractions.

Step 21: Final Lyophilization and Analys was: The collected fractions were analyzed by analytical RP-HPLC, and all fractions >95% purity are combined. Lyophilization of the combined fractions gave the title compound as a white powder with a purity >95% as determined by RPHPLC. Peptide identity was confirmed with LC/MS of the purified title compound, giving 2 charged states of the peptide, M+2/2 of 969 amu and the molecular ion of 1936 amu.

### Example 1F. Ac—O2R-Pen-Q-T-W-Q-Pen-Phe[4-(2-aminoethoxy)]-[2-Nal]-[THP]-O2R—N-[bA]-NH2

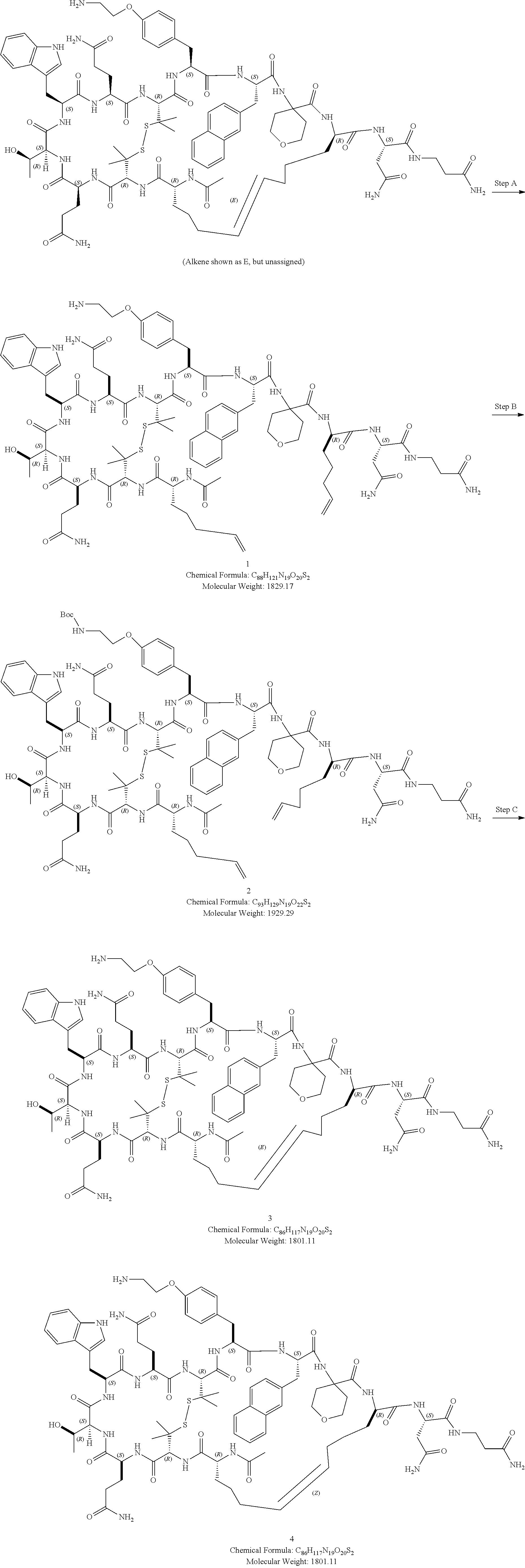

Synthesis of compound 1 was performed using Fmoc-protected amino acids on a solid-phase Rink Amide MBHA (NovaBiochem, 0.33 meq/g, 100-200 mesh) with a CEM Liberty Blue automated microwave peptide synthesizer. Peptide was synthesized on a 0.22 mmol scale. First residue (bAla) was incorporated manually using 3 eq of amino acid, 3 eq of HOAt and 3 eq of DIC in NMP, at RT overnight. Typical reaction conditions were as follows: Deprotection Conditions: 20% piperidine (v/v) in DMF (2 min at 90° C.); Residue Coupling Conditions: protected amino acid (2.5 mL of a 0.4 M amino acid stock solution in DMF) was delivered to the resin, followed by DIC activator (2 mL of a 0.5 M solution in DMF), and Oxyma Pure (1 mL of a 1 M solution in DMF) and allowed to react for 2 min at 90° C. For 2Nal a double coupling was performed. Capping of the free amino group was performed using 10 eq of acetic anhydride in DMF.

At the end of the assembly, the peptide resin was washed with DMF, MeOH, DCM, Et₂O. The peptide was cleaved from solid support using 87.5% TFA, 5% phenol, 2.5% triisopropylsilane and 5% water for 1.5 hours at room temperature. The resin was filtered and then added to cold methyl-t-butyl ether in order to precipitate the peptide. After centrifugation, the peptide pellets were washed with fresh cold diethyl-ether to remove the organic scavengers. The process was repeated twice. Final pellets were dried, re-suspended in H2O and acetonitrile 1:1+0.1% TFA and stirred overnight. Then lyophilized to afford the desired protected intermediate compound 1 (Yield: 80.4%). LCMS anal. calc. For C88H121N19O20S2: 1829.17; found: 916.4 (M+2)²⁺.

The precipitated solid crude intermediate 13-1 was dissolved in water: acetonitrile (1 mg/ml). Saturated iodine in acetic acid was then added drop wise with stirring until yellow color persisted. The solution was stirred for 30 minutes, and the reaction was monitored with UPLC-MS. When the reaction was completed, solid ascorbic acid was added until the solution became clear. DIPEA was added until the solution became basic. 1.5 eq. of Boc anhydride was added. The solution was stirred for 60 minutes, and the reaction was monitored with UPLC-MS. The reaction mixture was quenched with CH₃COOH. The solvent mixture was then lyophilized and the resulting material was then dissolved in 2.5 ml of DMSO and was purified by C4 reverse-phase HPLC (Waters Deltapak C4 (40×200 mm, 15 μm, 300 Å), using as eluents (A) 0.1% TFA in water and (B) 0.1% TFA, gradient began with 30% B, and changed to 45% B over 20 minutes at a flow rate of 80 ml/min). Fractions containing pure product were collected and then freeze-dried to afford intermediate compound 2. (Yield: 40%). LCMS anal. calcd. For C93H129N19O22S2: 1929.29; found: 965.5 (M+2)²⁺.

Intermediate 2 was dissolved in dry DCE (1 mg/mL) with 5% AcOH. Grubbs 2 catalyst (0.25 eq) (CAS: 246047-72-3) was added, stirred at 60° C. under N₂ atmosphere, and monitored by UPLC-MS. Reaction was almost complete after 30 min, whereupon 0.2 more eq of catalyst added. After 2 h, reaction mixture was allowed to cool to RT and SilaMet DMT scavenger resin was added (loading: 0.57 mmol/g, 8 eq respect to the catalyst). Stirred overnight. Then, reaction mixture was concentrated to dryness under reduced pressure to obtain a mixture of isomers 3 and 4. The mixture was treated with 10 mL of solution (v/v) (95% TFA, 5% H₂O) to remove the Boc protecting group for 10 min., and then concentrated to dryness. The reaction crude was re-dissolved in 2 ml of DMSO and purified by a reverse phase HPLC (Phenomenex Luna C18, 30×250 mm, 5 μm, 100 Å). Mobile phase A: +0.1% TFA, mobile phase B: Acetonitrile (ACN)+0.1% TFA, gradient began with 20% B, and changed to 30% B over 25 minutes at a flow rate of 45 mL/min. Collected fractions containing first eluted isomer were then lyophilized to afford the first isomer of the title compound. LCMS anal. calcd. For C86H117N19O20S2: 1801.12; found: 1801.6 (M+1)⁺. Collected fractions containing second eluted isomer were then lyophilized to afford the second isomer 4. LCMS anal. calcd. For C86H117N19O20S2: 1801.12; found: 901.1 (M+2)²⁺.

### Example 1G. 7Ahp(2)-Pen(3)-N-T-7MeW-K(Ac)-Pen(3)-AEF-2Nal-THP-E(2)-N-3Pya-Sar-CONH2

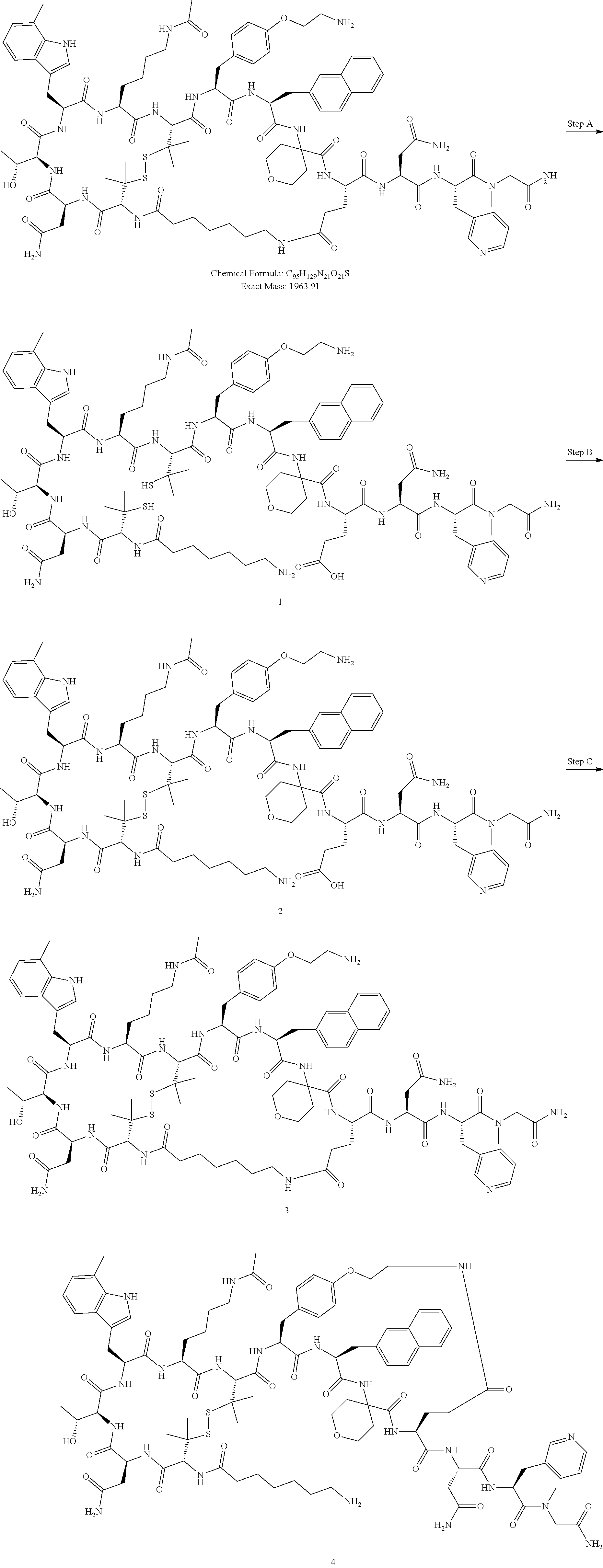

Linear peptides were synthesized using a CEM Liberty Blue automated microwave peptide synthesizer using standard Fmoc peptide synthesis with Rink Amide MBHA resin (NovaBiochem, 0.34 mmol/g, 100-200 mesh). Fmoc-protected amino acids (5 mL, 0.2 M, 1 mmol) were coupled using DIC (2 mL, 0.5 M, 1 mmol) and Oxyma Pure (1 mL, 1 M, 1 mmol) at 90° C. for 3.5 min. Double couplings were used for Sar, THP, and Thr and for residues incorporated after Sar, THP, and Thr. Fmoc deprotection was carried out using 20% piperidine (v/v) in DMF at 90° C. for 1 min. The peptide was deprotected and cleaved from solid support by treatment with 92.5% TFA, 2.5% triisopropylsilane, 2.5% 2,2′-(ethylenedioxy)diethanethiol (DODT), and 2.5% water for 30 min at 42° C. on a CEM Razor cleavage system. The resin was filtered and washed with TFA. The filtrate was concentrated and precipitated with cold methyl tert-butyl ether (MTBE). The mixture was centrifuged, and the pellet was washed with fresh cold MTBE. This was repeated twice. The peptide pellet was dried, re-dissolved in water/acetonitrile+0.1% TFA, and lyophilized overnight to afford the desired Intermediate 1 (yield: 97%). LCMS anal. calcd. for C₉₅H₁₃₃N₂₁O₂₂S₂: 1983.94; observed: 1984.9 (M+H)⁺, 993.0 (M+2H)²⁺.

The crude Intermediate 1 was dissolved in 30% acetonitrile/H₂O (2 mg/mL). Iodine in methanol (0.1 M) was added drop wise with stirring until the yellow color persisted. After stirring for ˜2 hours, the reaction was complete determined by HPLC. The reaction was quenched by addition of 1M ascorbic acid in water until the solution became clear. The solution was concentrated and lyophilized. The resulting pellet was dissolved in DMSO and purified by reverse phase on a Biotage Selekt (Biotage Sfar Bio C18 D—Duo 300 Å, 20 um, 25 g column) using eluents (A) 0.1% TFA in water and (B) 0.1% TFA in acetonitrile and a gradient of 20% B to 55% B over 10 CVs. Fractions containing pure product were collected and lyophilized to afford Intermediate 2. (Yield: 30.3%) LCMS anal. calc. for C₉₅H₁₃₁N₂₁O₂₂S2: 1983.35; found: 992.0 (M+2H)²⁺ and 1983.8 (M+H)⁺.

The purified Intermediate 2 was dissolved in DMF (0.005 M). To this solution was added PyBOP (2 eq) and DIEA (4 eq) and the reaction was stirred at room temperature. Once the reaction was complete as determined by LCMS, the reaction was concentrated and purified by reverse phase HPLC (Waters XSelect CSH Prep C18, 5 um OBD column, 19×150 mm, 25 mL/min) using eluents (A) 0.1% TFA in water and (B) 0.1% TFA in acetonitrile and a gradient of 26% B to 33% B over 10 min. Fractions containing pure product were collected and lyophilized to afford the title compound 3. (Yield: 13%) LCMS anal. calc. for C₉₅H₁₂₉N₂₁O₂₁S2: 1965.3; found: 1964.8 (M+H)⁺, 1986.8 (M+Na)⁺, and 983.0 (M+2H)²⁺.

### Example 1H. 6Ahx(2)-Abu(1)-N-T-W-Q-C(1)-AEF-2Nal-THP-E(2)-N-3Pya-Sar-CONH2

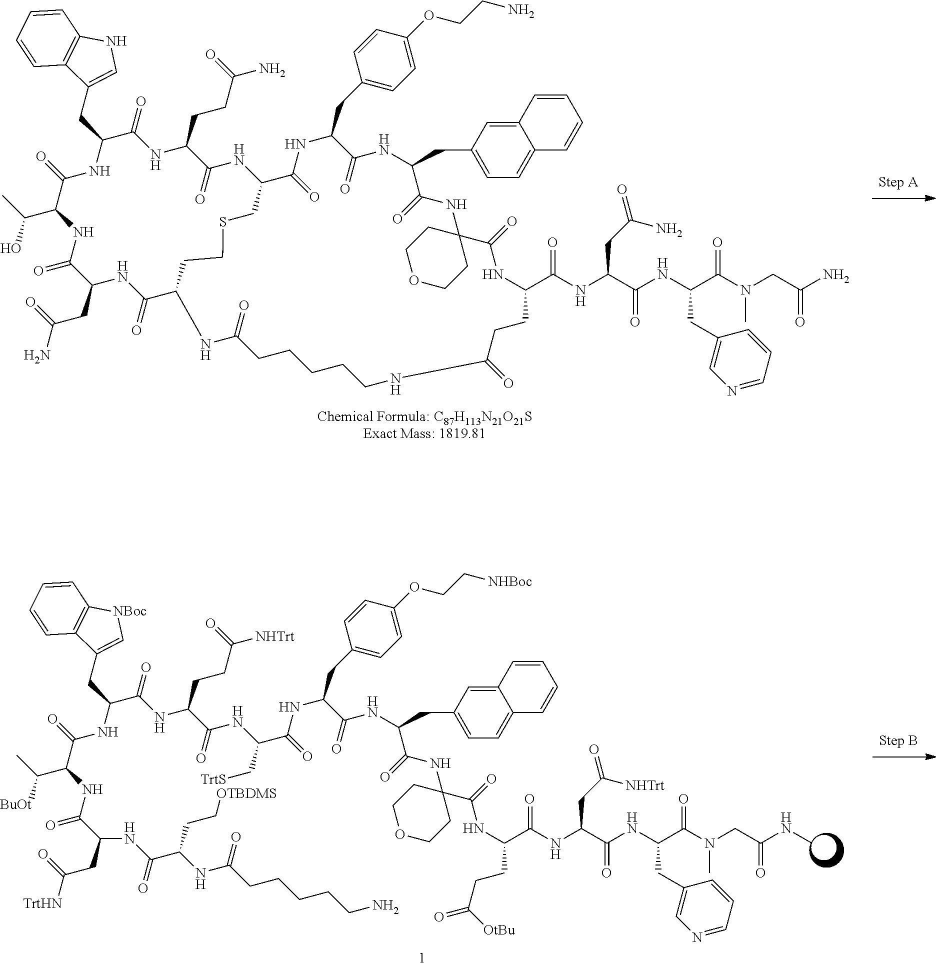

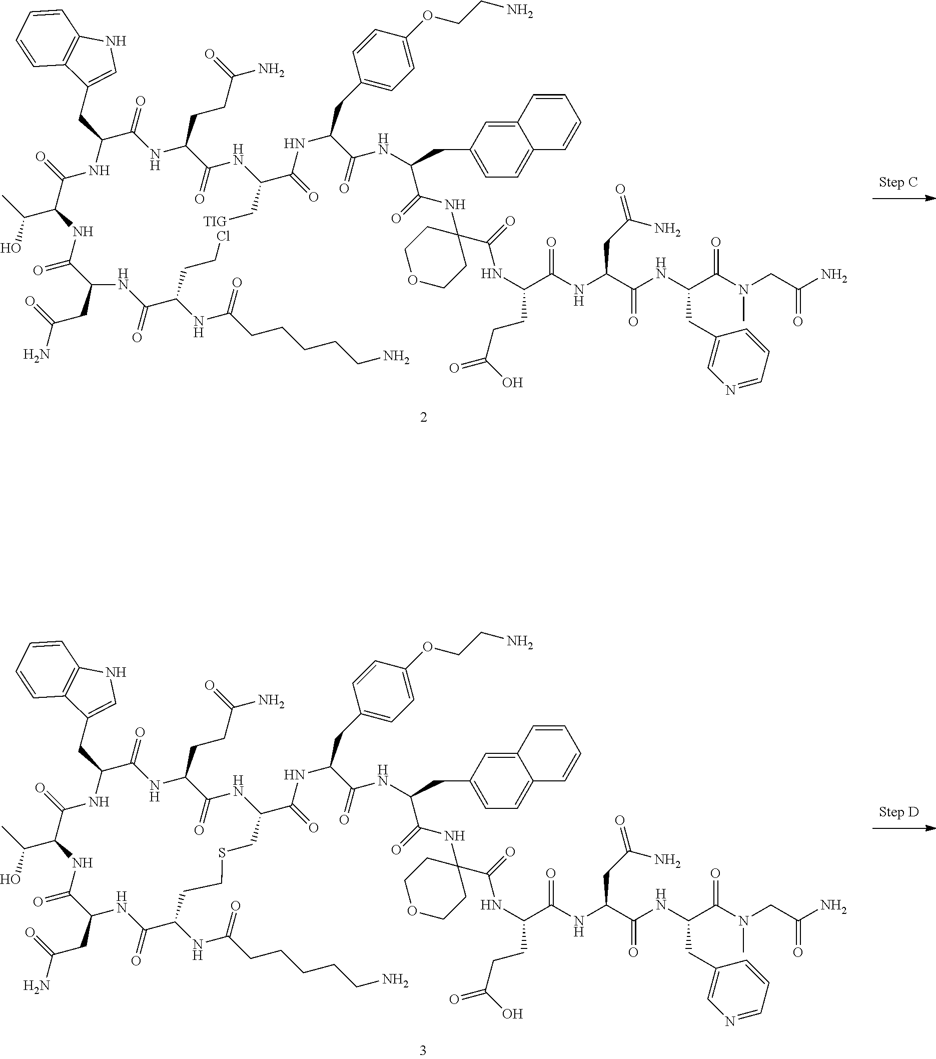

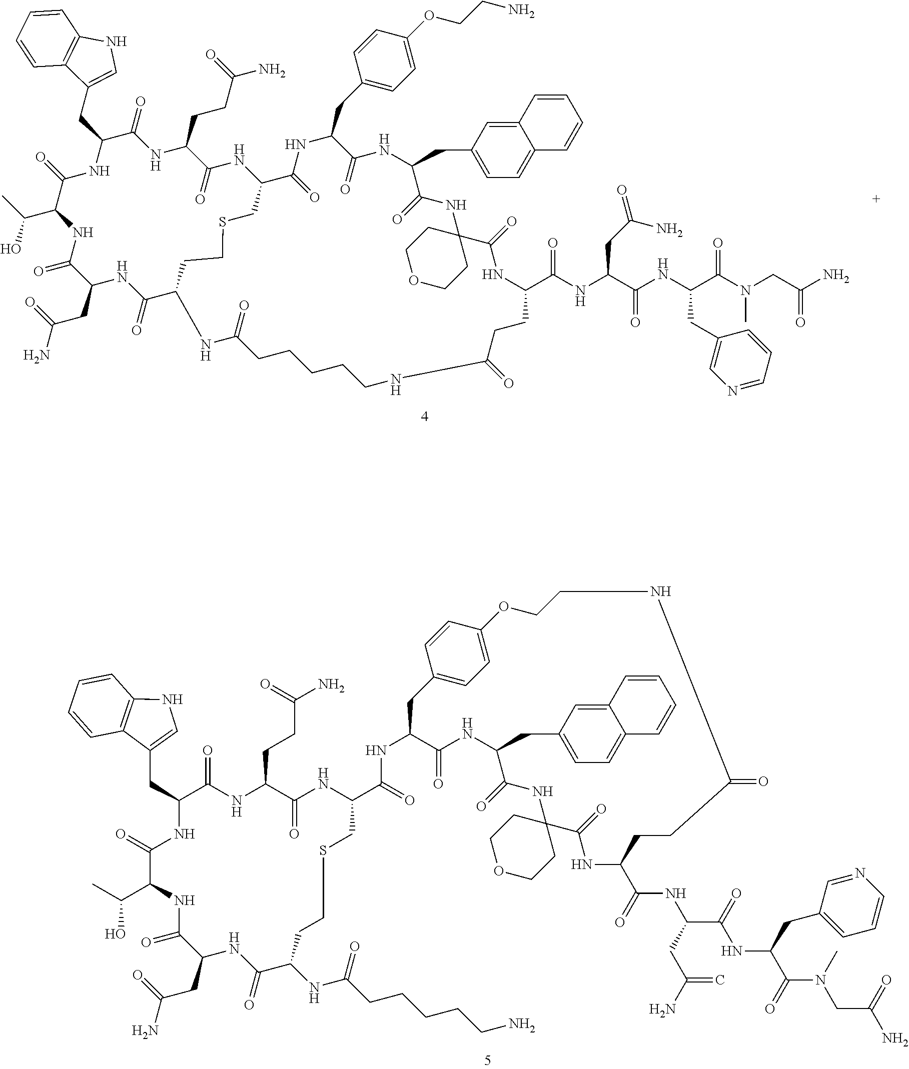

Linear peptide synthesis of 1 was conducted using a CEM Liberty Blue automated microwave peptide synthesizer using standard Fmoc peptide synthesis with Rink Amide MBHA resin (NovaBiochem, 0.34 mmol/g, 100-200 mesh). Fmoc-protected amino acids (5 mL, 0.2 M, 1 mmol) were coupled using DIC (2 mL, 0.5 M, 1 mmol) and Oxyma Pure (1 mL, 1 M, 1 mmol) at 90° C. for 3.5 min. Double couplings were used for Sar, THP, and Thr and for residues incorporated after Sar, THP, and Thr. Fmoc deprotection was carried out using 20% piperidine (v/v) in DMF at 90° C. for 1 min.

The resin-bound peptide Intermediate 1 was treated with a solution of dichlorotriphenylphosphorane (10 eq), α-pinene (15 eq), and thioanisole (15 eq) in dry DCM for 15 minutes. The resin was drained and washed with DCM. A fresh solution of dichlorotriphenylphosphorane (10 eq), α-pinene (15 eq), and thioanisole (15 eq) in dry DCM was added and the mixture was incubated on a rotary shaker for 3 hours. The resin was washed with DMF and DCM. The peptide was deprotected and cleaved from solid support by treatment with 92.5% TFA, 2.5% triisopropylsilane, 2.5% 2,2′-(ethylenedioxy)diethanethiol (DODT), and 2.5% water for 2 hours at room temperature. The resin was filtered and washed with TFA. The filtrate was concentrated and precipitated with cold methyl tert-butyl ether (MTBE). The mixture was centrifuged, and the pellet was washed with fresh cold MTBE. This was repeated twice. The peptide pellet was dried, re-dissolved in water/acetonitrile+0.1% TFA, and lyophilized overnight to afford Intermediate 2 (yield: 76%). LCMS anal. calcd. for Cs₇H₁₁₆ClN₂₁O₂₂S: 1873.80; observed: 938.5 (M+2H)²⁺.

The crude peptide was dissolved in DMF (2 mg/mL). NaI (1.5 eq) and EDTA (1.5 eq) were added to the peptide solution as 10 mg/mL solutions, followed by 0.1 M Na₂CO₃ (10 eq). The reaction was stirred overnight at room temperature, was quenched with TFA, concentrated, and purified by reverse phase on an ISCO (Biotage Sfar Bio C18 D—Duo 300 Å, 20 um, 25 g column) using eluents (A) 0.1% TFA in water and (B) 0.1% TFA in acetonitrile and a gradient of 20% B to 55% B over 10 CVs. Fractions containing pure product were collected and lyophilized to afford Intermediate 3. (yield: 21%) LCMS anal. calcd. for Cs₇Hn₅N₂₁O₂₂S: 1837.82; observed: 1838.8 (M+H)⁺, 1860.7 (M+Na)⁺, 920.0 (M+2H)²⁺.

The purified peptide was dissolved in DMF (0.005 M). To this solution was added PyBOP (2 eq) and DIEA (4 eq) and the reaction was stirred at room temperature. Once the reaction was complete as determined by LCMS, the reaction was concentrated and purified by reverse phase HPLC (Waters XSelect CSH Prep C18, 5 um OBD column, 19×150 mm, 25 mL/min) using eluents (A) 0.1% TFA in water and (B) 0.1% TFA in acetonitrile and a gradient of 20% B to 28% B over 10 min. Fractions containing pure product were collected and lyophilized to afford the title compound. (Yield: 5%) LCMS anal. calcd. for Cs₇H₁₁₃N₂₁O₂₁S: 1819.81; observed: 1820.8 (M+H)⁺, 1842.7 (M+Na)⁺, 911 (M+2H)²⁺.

### Example 1 I. McCO-Glu-Pen-N-T-7MeW-K(Ac)-Pen-AEF-2Nal-THP—K(Ac)—N-3Pva-Sar-CONH2

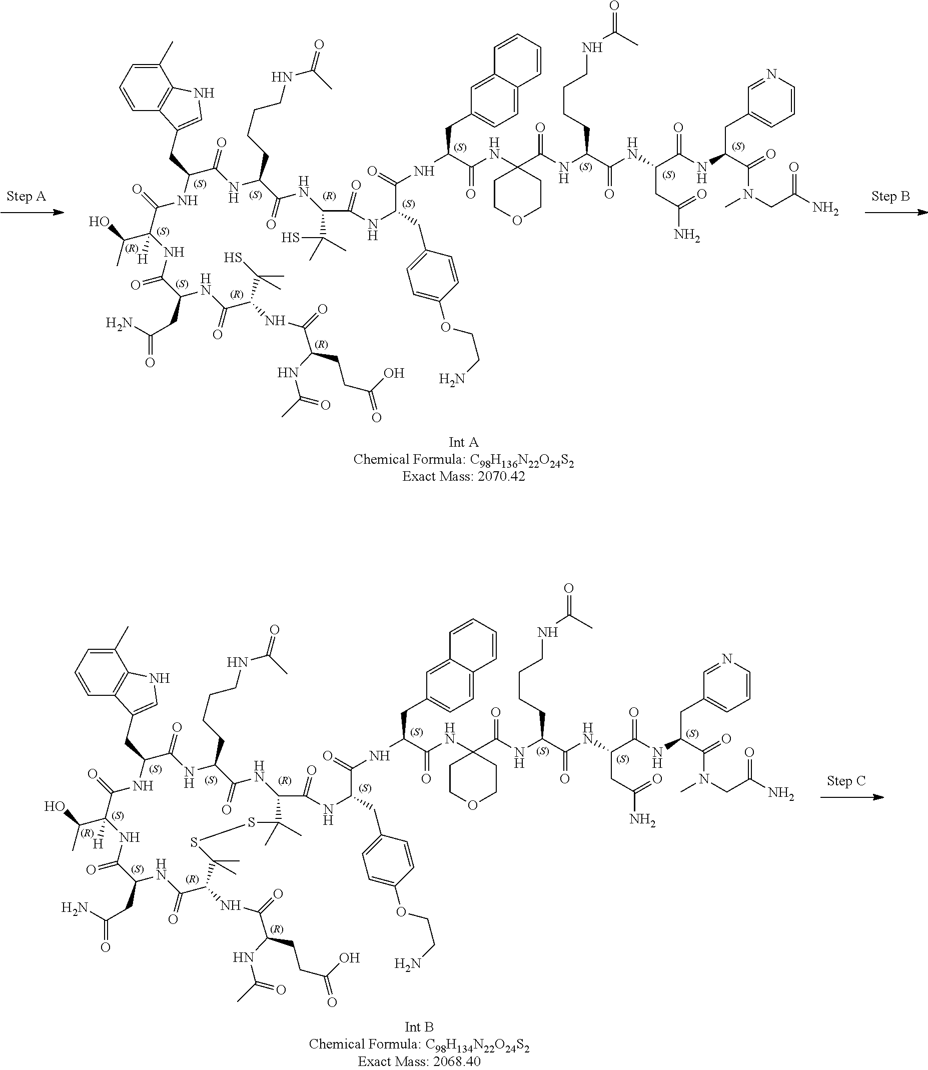

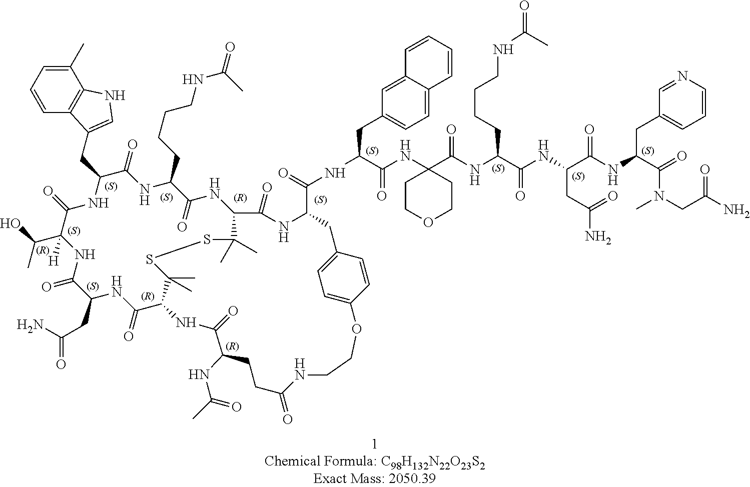

The peptide was synthesized by standard Solid-phase Peptide Synthesis (SPPS) using Fmoc/t-Bu chemistry. The assembly was performed on a Rink-amide AM resin (220 mol, 100-200 Mesh; loading 0.35 mmol/g) on the Cem Liberty Blue microwave peptide synthesizer (CEM Inc.). During peptide assembly on solid phase, the side chain protecting groups were: tert-butyl for Thr and Glu; trityl for Pen and Asn; tert-butoxy-carbonyl for AEF. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya15 & 2Nal10. Fmoc deprotections were performed using 20% (V/V) piperidine in DMF. Capping of the free amino group was performed manually using 10 eq of acetic anhydride in DMF.

At the end of the assembly the resin was washed with DMF, MeOH, DCM, Et₂O. The peptide was cleaved from solid support using 30 ml of TFA solution (v/v) (87.5% TFA, 5% H2O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H2O and acetonitrile 1:1+0.1% TFA and stirred overnight, then lyophilized to afford the desired linear Intermediate 166-1 (yield 83.4%). LCMS anal. calc. For C98H136N22O24S2: 2070.42 Da; found; 1036.6 (M+2)²⁺.

The crude peptide was dissolved in CAN/H₂O (5 mg/ml). Saturated iodine in acetic acid was then added dropwise under stirring until yellow color persisted. Rxn was completed in 20 min (monitored by UPLC-MS). Solid ascorbic acid was added until the solution became clear. After lyophilization, crude Intermediate 166-2 was used as such in the next step. LCMS anal. calc. For C98H134N22O24S2: 2068.40 Da; found; 1035.4 (M+2)²⁺.

Intermediate 166-2 was dissolved in DMF (0.5 mg/ml). HATU (2 Eq) and Dipea (4 Eq) were added. Reaction was left under stirring at room temperature for 60 min (monitored by UPLC-MS). Solvent was evaporated in vacuum. Purified by reverse-phase HPLC using preparative Waters DeltaPak C18 (200×40 mm, 100 Å, 15 μm). Mobile phase A: +0.1% TFA, mobile phase B: Acetonitrile (ACN)+0.1% TFA. The following gradient of eluent B was used: 20% B to 20% B over 5 min, to 35% B over 25 min, flow rate 80 mL/min, wavelength 214 nm. Collected fractions were lyophilized to afford the title compound 1 (yield 10%). LCMS anal. calc. For C98H132N22O23S2: 2050.39 Da; found; 1025.84 (M+2)²⁺.

### Example 1 J. McCO-k(2)-Pen(3)-O-T-W-Q-Pen(3)-AEF-2Nal-THP-E(2)-N-bAla-CONH2

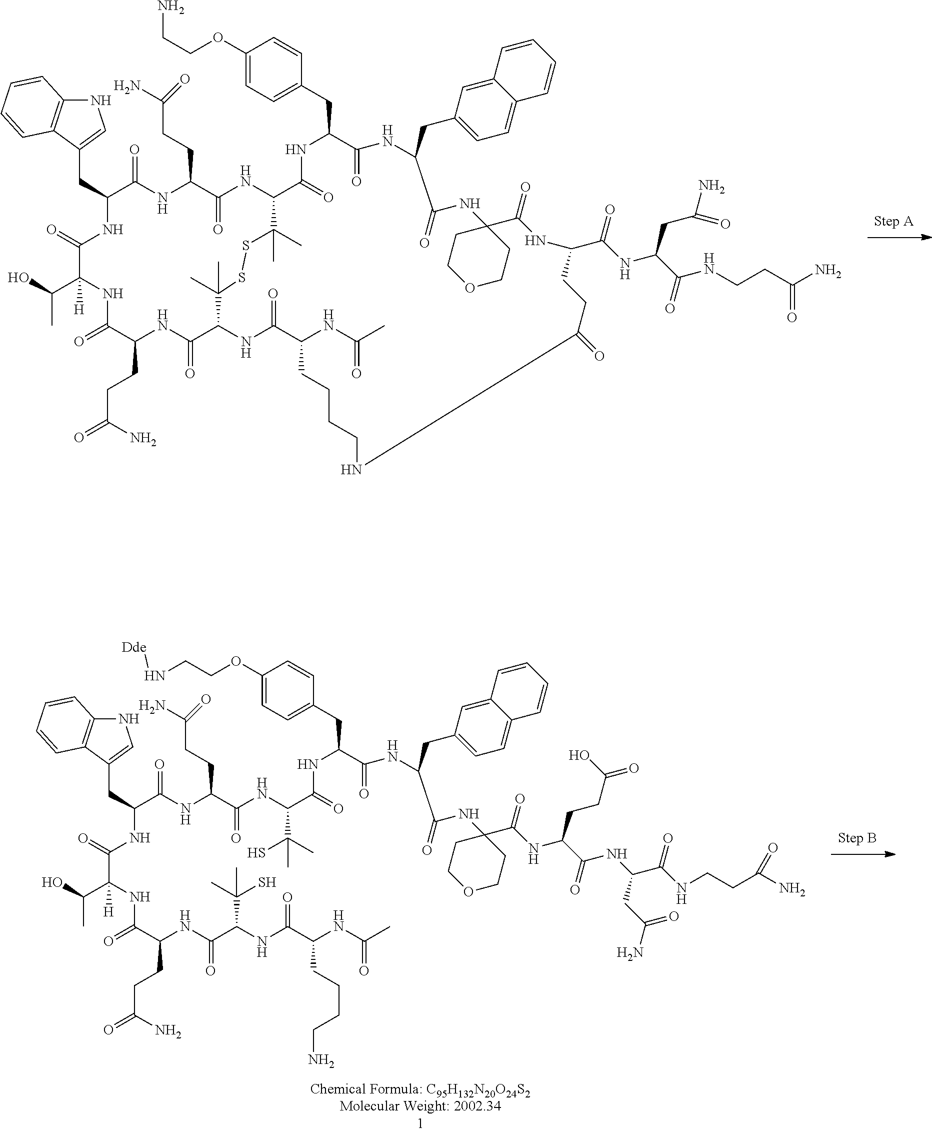

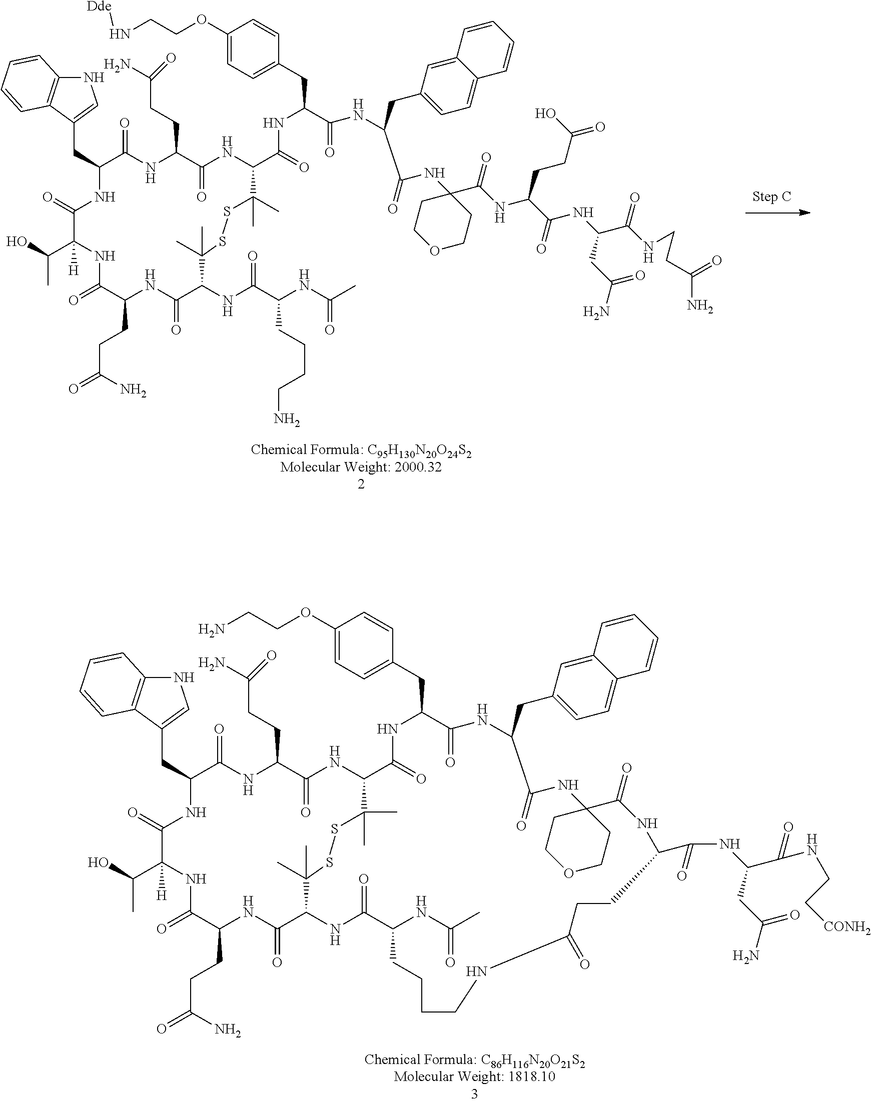

Step A—Synthesis of Intermediate Compound 1 Synthesis was performed using Fmoc-protected amino acids on a solid-phase Rink Amide MBHA (NovaBiochem, 0.33 meq/g, 100-200 mesh) with a CEM Liberty Blue automated microwave peptide synthesizer. Peptide was synthesized on a 0.22 mmol scale. First residue (bAla) was incorporated manually using 3 eq of amino acid, 3 eq of HOAt and 3 eq of DIC in NMP, at RT overnight. Typical reaction conditions were as follows: Deprotection Conditions: 20% piperidine (v/v) in DMF (2 min at 90° C.); Residue Coupling Conditions: protected amino acid (2.5 mL of a 0.4 M amino acid stock solution in DMF) was delivered to the resin, followed by DIC activator (2 mL of a 0.5 M solution in DMF), and Oxyma Pure (1 mL of a 1 M solution in DMF) and allowed to react for 2 min at 90° C. For 2Nal a double coupling was performed. Capping of the free amino group was performed using 10 eq of acetic anhydride in DMF.

At the end of the assembly, the peptide resin was washed with DMF, MeOH, DCM, Et2O. The peptide was cleaved from solid support using 87.58% TFA, 5% phenol, 2.5% triisopropylsilane and 5% water for 1.5 hours at room temperature. The resin was filtered and then added to cold methyl-t-butyl ether in order to precipitate the peptide. After centrifugation, the peptide pellets were washed with fresh cold diethyl-ether to remove the organic scavengers. The process was repeated twice. Final pellets were dried, re-suspended in H2O and acetonitrile 1:1+0.1% TFA and stirred overnight. Then lyophilized to afford the desired protected intermediate compound 1 (Yield: 86%). LCMS anal. calcd. For C95H132N20O24S2: 2002.34; found: 1001.9 (M+2)2+

Step B—Synthesis of Intermediate Compound 2 The precipitated solid crude peptide from Step A was dissolved in water/acetonitrile 1:1 (1 mg/mL). Saturated Iodine in acetic acid was then added drop wise with stirring until yellow color persisted. The solution was stirred for 15 minutes, and the reaction was monitored UPLC-MS. When the reaction was completed, solid ascorbic acid was added until the solution became clear. The solvent mixture was then lyophilized and the resulting material was then dissolved in DMSO and purified by a reverse phase HPLC (Deltapak C4, 40×200 mm, 15 μm, 300 Å). Mobile phase A: +0.1% TFA, mobile phase B: Acetonitrile (ACN)+0.1% TFA, gradient began with 20% B, and changed to 35% B over 25 minutes at a flow rate of 80 mL/min. Collected fractions containing pure product were then lyophilized to afford compound 2 (Yield: 48%). LCMS anal. calcd. For C95H130N20O24S2: 2000.32; found: 1001.1(M+2)2+

Step C—Synthesis of Intermediate Compound 3 Compound 2 was dissolved in DMF (0.5 mg/mL). HATU (1.1 eq) and DIPEA (3 eq) in DMF (5 mL) were added dropwise. The resulting solution was stirred for 5 min at room temperature (monitored by UPLC-MS). After completion of the cyclization, hydrazine monohydrate (20 eq) was added to remove Dde protecting group. Deprotection was complete after 30 min (monitored by UPLC-MS). The reaction mixture was quenched with TFA and concentrated to dryness. Reaction crude was re-dissolved in 4 ml of DMSO and purified in two runs by a reverse phase HPLC (Deltapak C18, 40×200 mm, 15 μm, 100 Å). Mobile phase A: +0.1% TFA, mobile phase B: Acetonitrile (ACN)+0.1% TFA, gradient began with 20% B, and changed to 35% B over 25 minutes at a flow rate of 80 mL/min. Collected fractions containing pure product were then lyophilized to afford Compound 3 (Yield: 49%). LCMS anal. calcd. For C85H116N20O21S2: 1818.10; found: 909.9 (M+2)2+

### Example 1 K. PEG2(2)-Pen(3)-N-T-7MeW-K(Ac)-Pen(3)-AEF-2Nal-THP-hE(2)-N-3Pya-Sar-CONH2

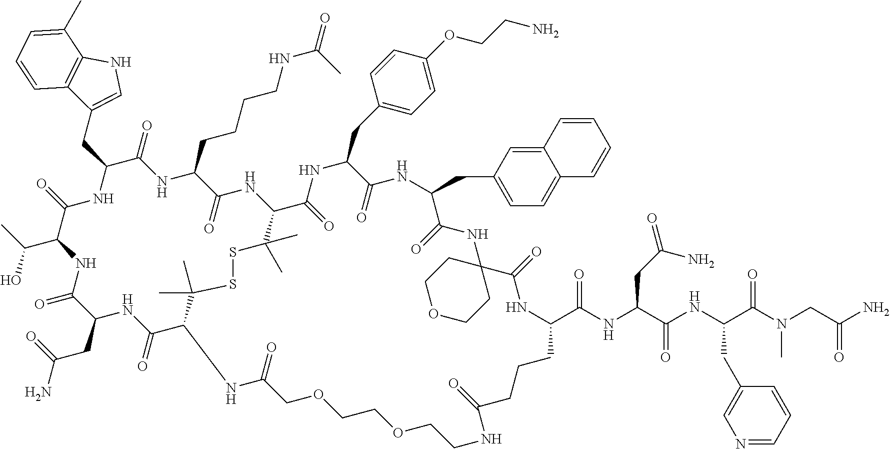 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)COCCOCCNC(=O)CCC[C@@H](C(=O)N[C@@H](CC(N)=O)C(=O)N[C@@H](Cc3cccnc3)C(=O)N(C)CC(N)=O)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C]

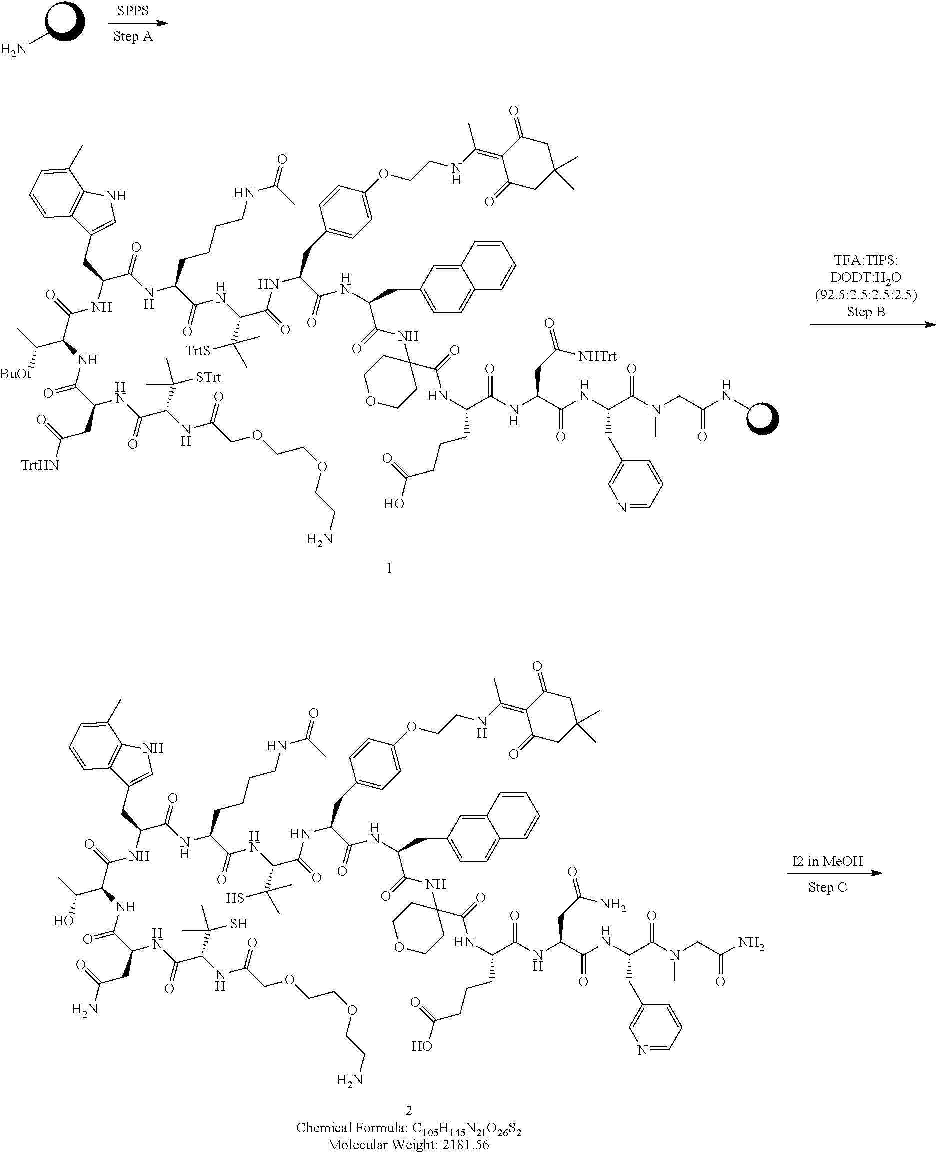

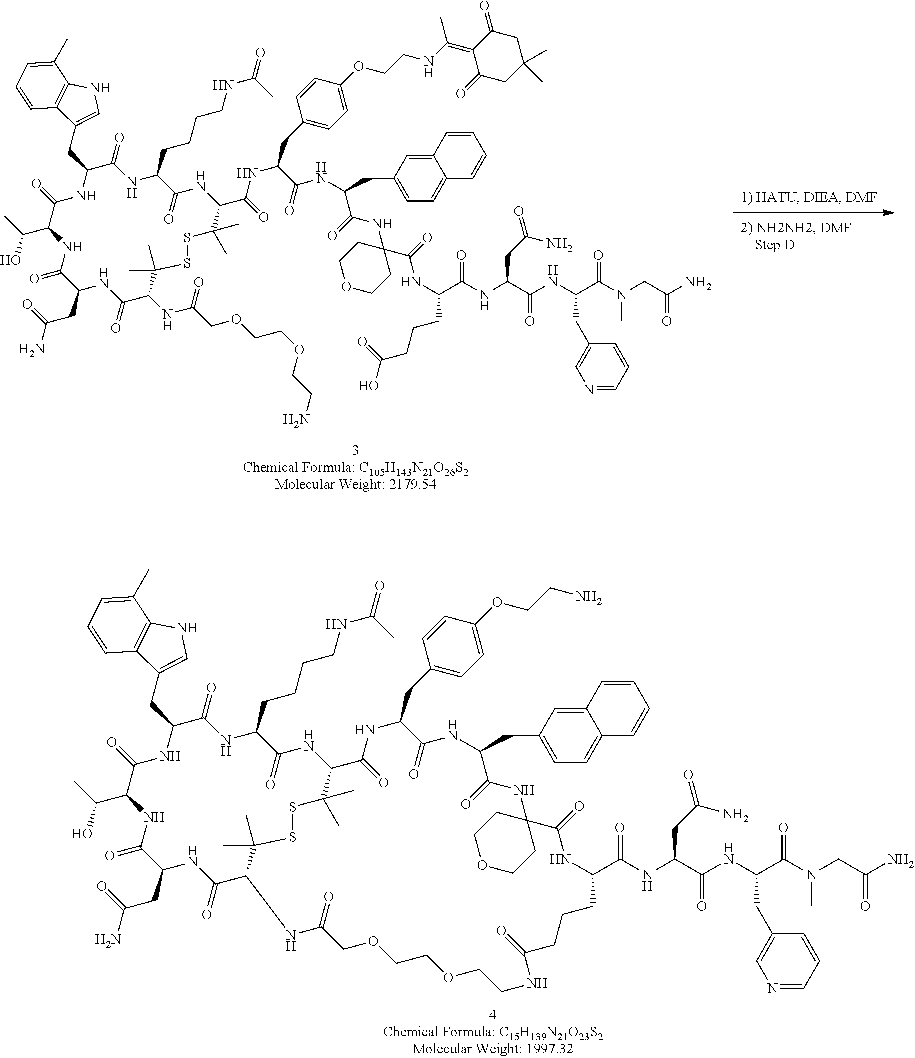

Step A—Synthesis of Intermediate Compound 1 Synthesis was performed using standard Fmoc solid phase peptide synthesis on Rink Amide MBHA LL resin (NovaBiochem, 0.34 mmol/g, 100-200 mesh). The resin-bound peptide was synthesized on a CEM Liberty Blue automated microwave peptide synthesizer at 1 mmol scale. Typical reaction conditions were as follows: Deprotection Conditions: 20% piperidine (v/v) in DMF (10 mL, 1.5 min at 90° C.); Residue Coupling Conditions: protected amino acid (5 mL of 0.4 M amino acid stock solution in DMF) was delivered to the resin, followed by DIC activator (2 mL of a 0.5 M solution in DMF), and Oxyma Pure (1 mL of a 1 M solution in DMF) and allowed to react for 3.5 min at 90° C. Double couples were performed for Sar, 3Pya, THP, and 2Nal. A manual coupling for AEF(Dde) was performed. Fmoc-AEF(Dde)-OH (1.5 eq) was activated with HOAt (1.5 eq) and DIC (1.5 eq) in DMF for 20 min, then added to the resin and mixed at room temperature for 16 hrs.

Step B—Synthesis of Intermediate Compound 2 After complete assembly, the peptide resin was washed with DMF, MeOH, DCM. The peptide was deprotected and cleaved from solid support by treating the resin with 92.5% TFA, 2.5% water, 2.5% triisopropylsilane (TIPS), and 2.5% 3,6-dioxa-1,8-octanedithiol (DODT) for 30 min at 42° C. on a CEM Razor cleavage station. The resin was filtered and washed with TFA. The mixture was concentrated and added to cold methyl-t-butyl ether to precipitate the peptide. After centrifugation, the peptide pellet was washed with fresh cold methyl-t-butyl ether. This was repeated once more. Final pellets were dried, re-suspended in water and acetonitrile (1:1)+0.1% TFA and lyophilized to afford desired intermediate 2 (Yield: 89.7%) LCMS anal. calc. for C₁₀₅H₁₄₅N₂₁O₂₆S₂: 2181.56; found: 1091.3 (M+2H)²⁺ and 727.8 (M+3H)³⁺.

Step C—Synthesis of Intermediate Compound 3 Intermediate 2 was dissolved in water:acetonitrile (1:1) (1 mg/mL). Iodine in methanol (0.1 M) was added dropwise with stirring until the yellow color persisted. The reaction was monitored by HPLC-MS. When the reaction was complete, ascorbic acid in water (1 M) was added until the solution became clear. The reaction was concentrated and lyophilized. The crude material was dissolved in DMSO and purified by reverse-phase on an ISCO (Biotage Sfar Bio C18 D—Duo 300 Å, 20 uM, 50 g column, 40 mL/min) using eluents (A) 0.1% TFA in water and (B) 0.1% TFA in acetonitrile and a gradient of 20% B to 55% B over 10 CVs. Fractions containing pure product were collected and lyophilized to afford intermediate 3. (Yield: 32%) LCMS anal. calc. for C₁₀₅H₁₄₃N₂₁O₂₆S₂: 2119.55; found: 1090.4 (M+2H)²⁺ and 727.1 (M+3H)³⁺.

Step D—Synthesis of Compound 4 Intermediate 3 (292.1 mg, 0.121 mmol) was dissolved in DMF (25 mL, 0.005 M). To this solution was added HATU (69.2 mg, 0.182 mmol) and N,N-Diisopropylethylamine (84.5 uL, 0.485 mmol) and stirred at room temperature. The reaction was monitored by HPLC-MS. Once the reaction was complete, hydrazine (38.9 uL, 1.21 mmol) was added and stirred at rt for 1 hr. The mixture was concentrated, dissolved in DMSO and purified by reverse-phase HPLC (Waters XSelect CSH Prep C18, 5 um OBD column, 19×150 mm, 25 mL/min) using eluents (A) 0.1% TFA in water and (B) 0.1% TFA in acetonitrile and a gradient of 22% B to 27% B over 10 min. Fractions containing pure product were collected and lyophilized to afford compound 3. (Yield: 14%) LCMS anal. calc. for C₉₅H₁₂₉N₂₁O₂₃S₂: 1997.33; found: 1996.6 (M+H)⁺ and 998.9 (M+2H)²⁺.

### Example 1 L. McCO-Pen(3)-K(NMe)(5)-T-7MeW-K(Ac)-Pen(3)-AEF(5)-2Nal-THP-E-N-3Pya-Sar-CONH2

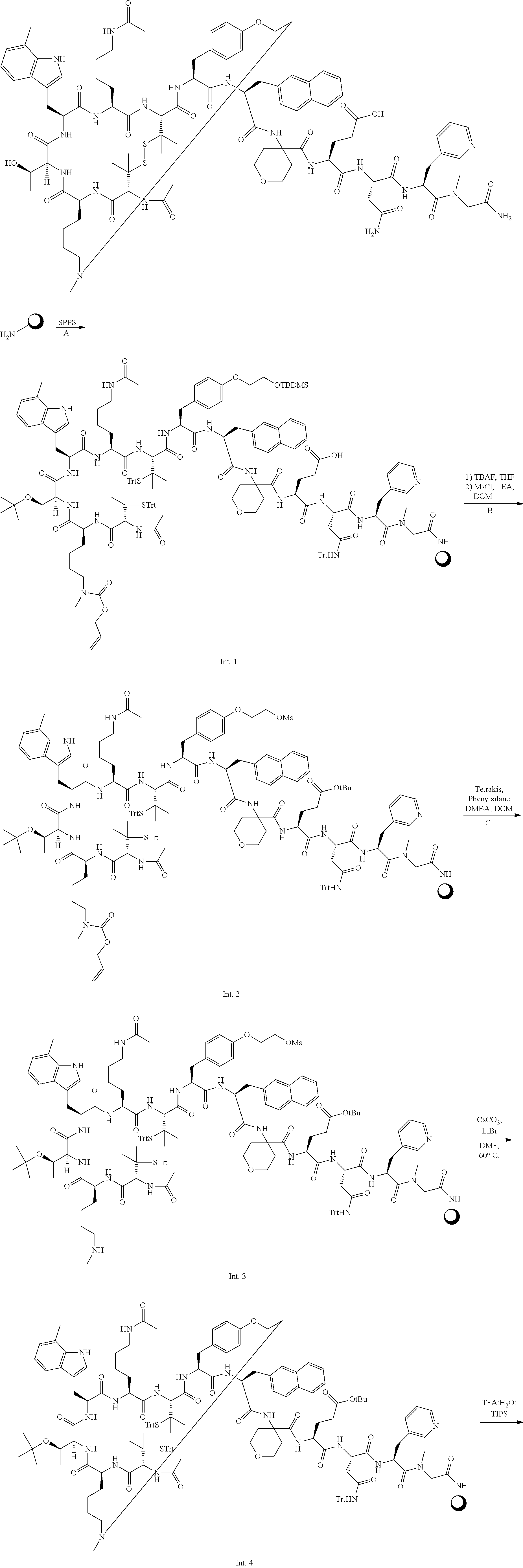

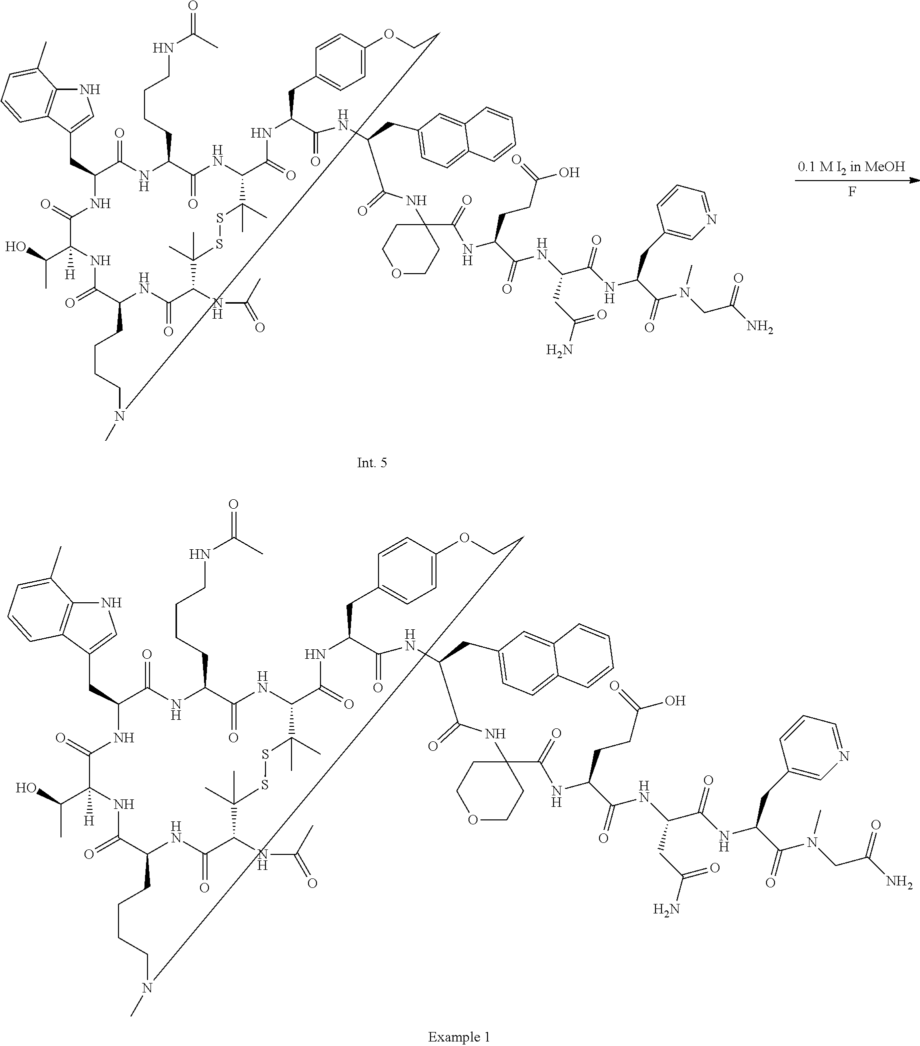

Step A—Synthesis of Intermediate Compound 1—Synthesis was performed using Fmoc-protected amino acids on a solid-phase Rink amide MBHA resin (Novabiochem, 0.42 mmol/g, 100-200 mesh) with a Biotage Syro II parallel peptide synthesizer. Peptide was synthesized on a 0.05 mmol scale. Typical reaction conditions were as follows: Deprotection Conditions: Fmoc deprotection was carried out in 2 stages using 40% piperidine in DMF (1 mL) under room temperature for 3 minutes, followed by 20% piperidine in DMF (1 mL) for 9 minutes. Residue Coupling Conditions: Fmoc-protected amino acid (0.5 mL of a 0.5 M amino acid stock solution in DMF, 0.25 mmol) was delivered to the rein, followed by HATU (0.52 mL of a 0.48 M stock solution in DMF, 0.25 mmol), and 4-methylmorpholine (0.25 mL, 2M, 0.5 mmol) and allowed to react for 1 hour at room temperature. Residue [Y(OEtOTBDMS)] was coupled using manual coupling conditions: the mixture of Fmoc-protected amino acid (0.125 mmol), HATU (0.125 mmol) and 4-methylmorpholine (0.35 mmol) in DMF (8 mL) was added to the resin (0.05 mmol) and then mixed for 2 hours at room temperature. The peptide was capped with Ac2O/NMM/DMF (1:1:3) (1 mL).

Step B—Synthesis of Intermediate Compound 2—Intermediate 1 (0.15 mmol) was swelled in THF (8 mL) for 15 min, then TBAF (1.5 mL, 1 M in THF, 1.5 mmol) was added. The reaction was mixed at room temperature for 1 hr. The resin is then drained, washed with DMF (8 mL, 3 times) and DCM (8 mL, 3 times). To the resulting resin in DCM (10 mL) was added TEA (0.834 mL, 0.728 g/mL, 6 mmol) in DCM (5 ml) and then followed by the slow addition of solution of METHANESULFONYL CHLORIDE (0.233 mL, 1.48 g/mL, 3 mmol) in DCM (5 mL). The reaction was mixed at room temperature for 1 hr, then drained, washed with DMF (3×) and DCM (3×). Microcleavage of the resin with TFA shows the desired product. LCMS anal. calc. for C₉₇H₁₃₃N₁₉O₂₄S₂ 2013.368; found: 1007.0 (M+2)²⁺

Step C—Synthesis of Intermediate Compound 3—To intermediate 2 (0.15 mmol) in DCM (5 mL) was added PHENYLSILANE (0.286 mL, 0.877 g/mL, 2.25 mmol) in DCM (2 mL) and 1,3-DIMETHYLBARBITURIC ACID (354.9 mg, 2.25 mmol) in DCM (2 mL) under N2 for 1-2 mins. TETRAKIS(TRIPHENYLPHOSPHINE)PALLADIUM(0) (87.5 mg, 0.075 mmol) in DCM (2 mL) was added and the reaction was mixed for 40 min at rt. The resin was drained and washed with DCM (8×). Microcleavage of the resin with TFA shows the desired product. LCMS anal. calc. for C₉₃H₁₂₉N₁₉O₂₂S₂ 1929.293; found: 965.0 (M+2)²⁺

Step D—Synthesis of Intermediate Compound 4—Intermediate 3 (0.15 mmol) was swelled in DMF (10 mL) for 15 min, then was added to a saturated Cs₂CO₃ solution of DMF (400 mL). Then lithium bromide (1302.6 mg, 15 mmol) was added and the reaction mixture was heated at 60° C. for 1 hr. The resin is then cooled to room temperature, drained, washed with water (3×), DMF (3×) and DCM (3×). Microcleavage of the resin with TFA shows the desired product. LCMS anal. calc. for C₉₃H₁₂₇N₁₉O₂₁S₂ 1911.278, found: 956.3 (M+2)²⁺

Step E—Synthesis of Compound 5—Intermediate compound 4 (0.15 mmol) was treated with a cocktail solution of TFA/H₂O/TIPS 92.5/5/2.5 for 30 mins at 42° C. on a CEM Razor cleavage station. The mixture was then concentrated and then added to cold methyl-t-butyl ether in order to precipitate the peptide. After centrifugation, the peptide pellets were washed with fresh cold methyl-t-butyl ether to remove the organic scavengers. The process was repeated twice. Final pellets were dried, re-suspended in H₂O and acetonitrile, then lyophilized to afford the desired protected intermediate Compound 5 as a light yellow solid. LCMS anal. calc. for C93H127N19O21S2: 1911.278 found: 956.3 (M+2)²⁺

Step F—Synthesis of Compound 1—The intermediate crude peptide 5 from Step E was dissolved in 40% ACN/water (50 mL). Iodine in methanol (0.1 M) was then added drop wise with stirring until yellow colour persisted. The reaction was monitored with UPLC-MS. When the reaction was complete, solid ascorbic acid was added until the solution became clear. The solvent mixture was then lyophilized, and the resulting material was then dissolved in DMSO and was purified by Prep HPLC. Fractions containing pure product were collected and then freeze-dried to afford the desired product as a white powder. LCMS anal. Calcd. for C₉₃H₁₂₅N₁₉O₂₁S₂ 1909.26; found: 955.0 (M+2)²⁺.

### Example 1 M. AEEP(5)-Pen(3)-N-T-7MeW-K(Ac)-Pen(3)-AEF-2Nal-THP-hSer(5)-N-3Pva-Sar-CONH2

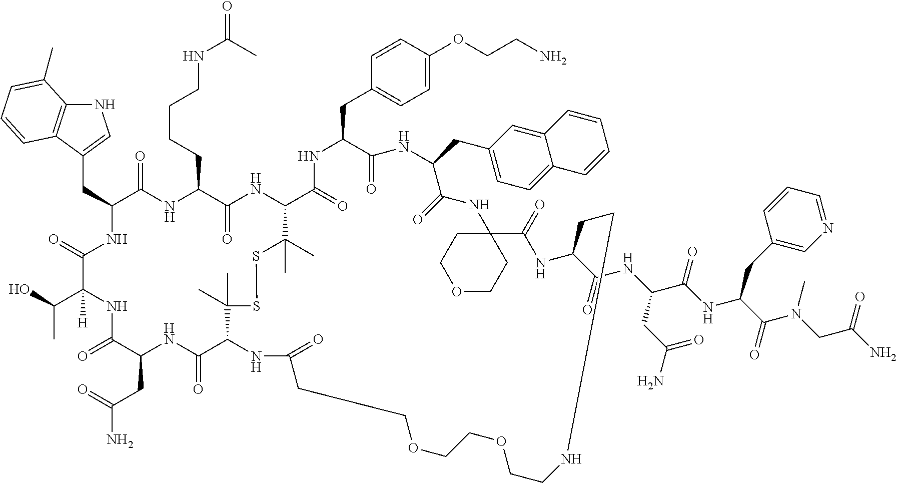 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)CCOCCOCCNCC[C@@H](C(=O)N[C@@H](CC(N)=O)C(=O)N[C@@H](Cc3cccnc3)C(=O)N(C)CC(N)=O)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C]

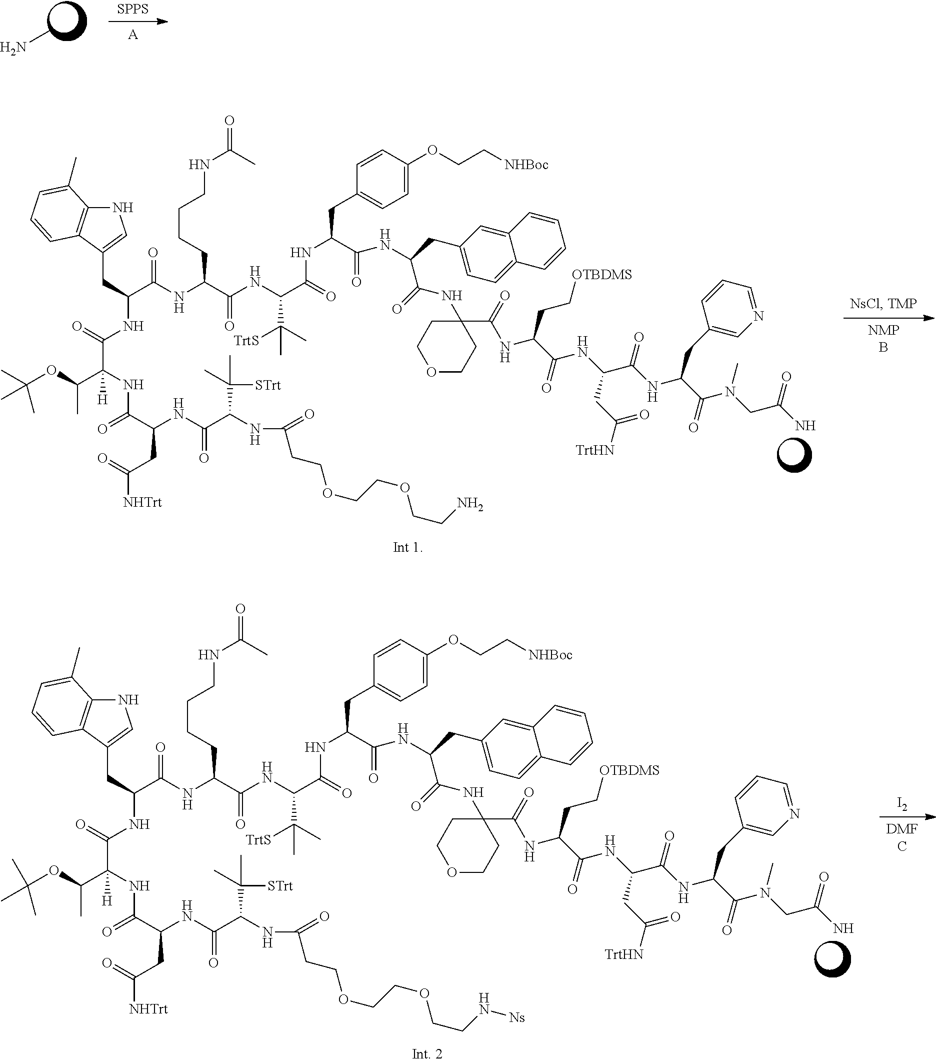

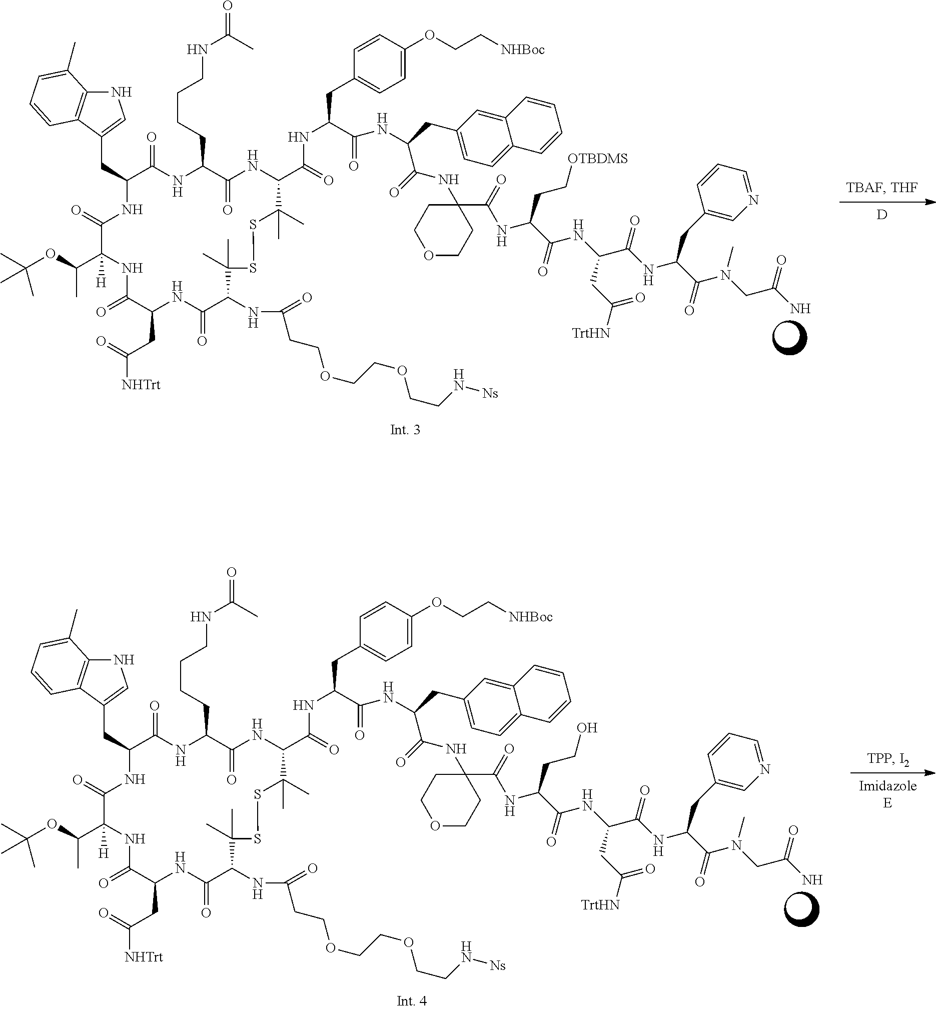

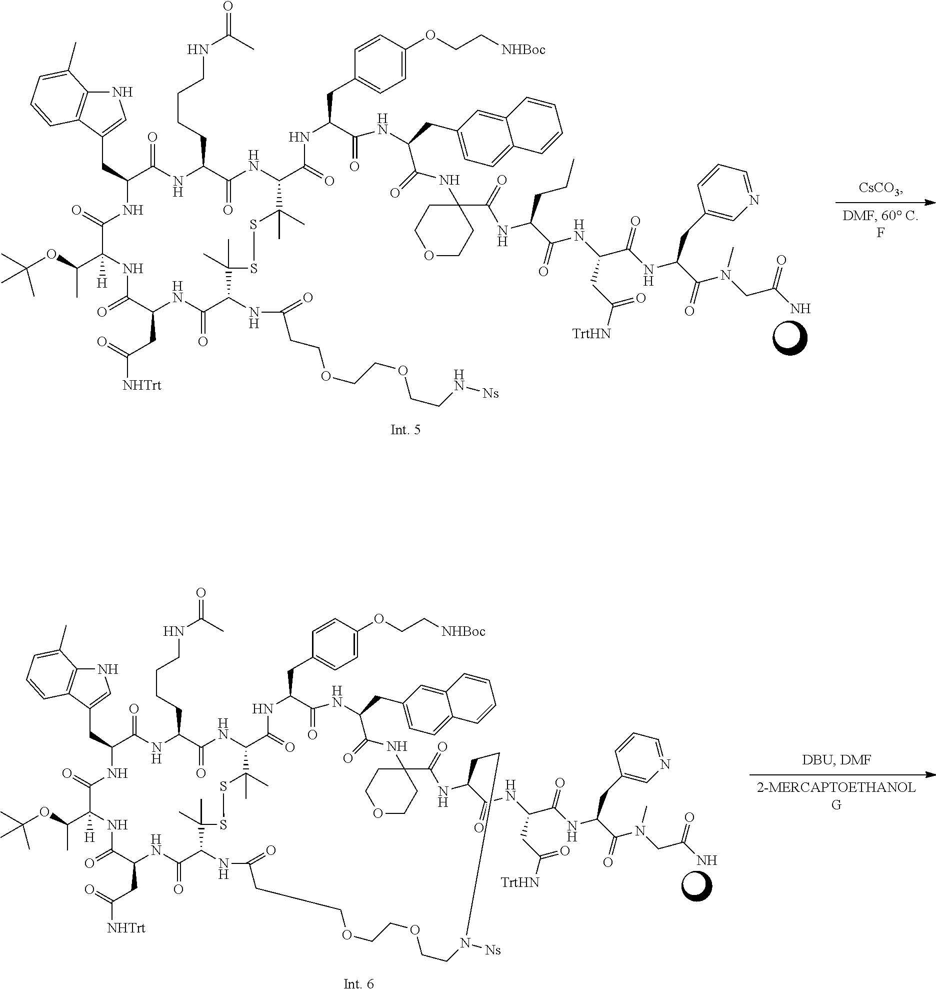

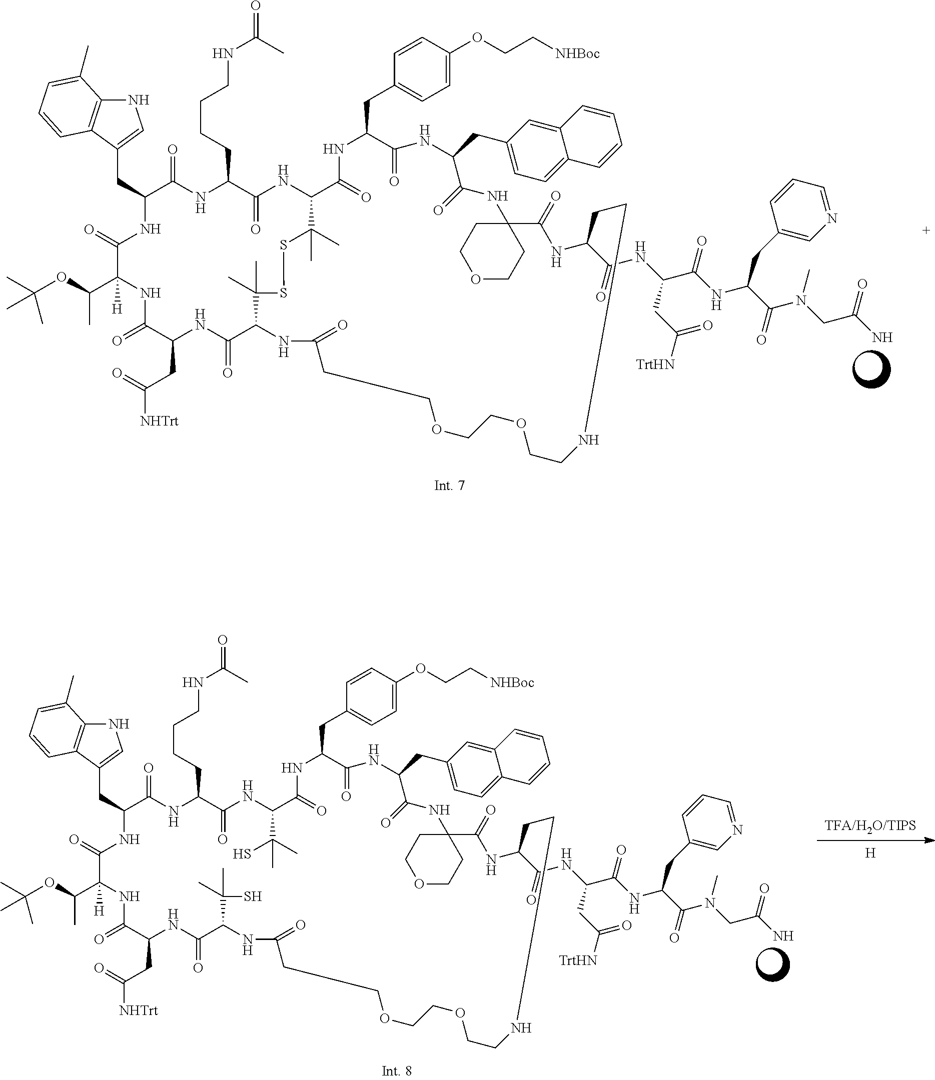

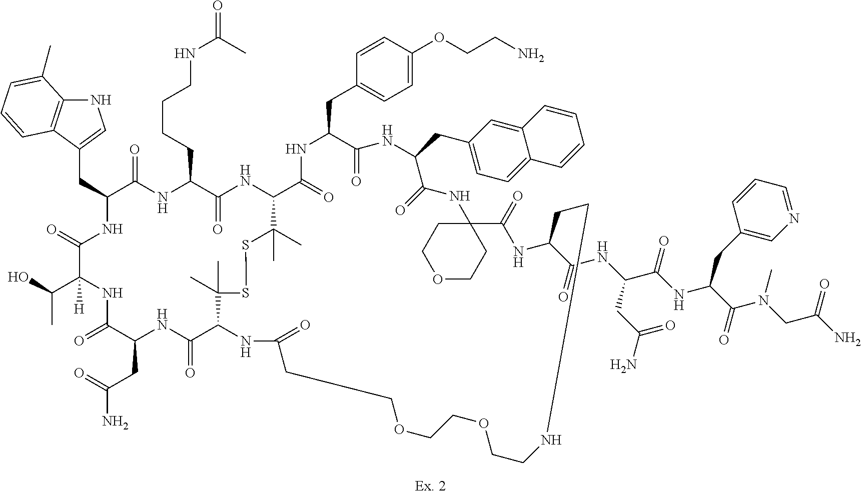 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H]2NC(=O)CCOCCOCCNCC[C@@H](C(=O)N[C@@H](CC(N)=O)C(=O)N[C@@H](Cc3cccnc3)C(=O)N(C)CC(N)=O)NC(=O)C3(CCOCC3)NC(=O)[C@H](Cc3ccc4ccccc4c3)NC(=O)[C@H](Cc3ccc(OCCN)cc3)NC(=O)[C@@H](NC1=O)C(C)(C)SSC2(C)C]

Step A—Syntheis of Intermediate 1—Synthesis was performed using Fmoc-protected amino acids on a solid-phase Rink amide MBHA resin (Novabiochem, 0.42 mmol/g, 100-200 mesh) with a CEM Liberty Blue automated microwave peptide synthesizer. Peptide was synthesized on a 0.25 mmol scale. Typical reaction conditions were as follows: Deprotection Conditions: Fmoc deprotection was carried out using 20% piperidine in DMF (10 mL) under microwave conditions (90° C., 1 min). Residue Coupling Conditions: Fmoc-protected amino acid (5 mL of a 0.2 M amino acid stock solution in DMF, 1 mmol) was delivered to the rein, followed by N,N′-DIISOPROPYLCARBODIIMIDE (2.041 mL, 0.5 M, 1 mmol) and ETHYL (HYDROXYIMINO)CYANOACETATE (1 mL, 1 M, 1 mmol) at 90° C. for 3.5 min. Double couplings were used for 3Pya, THP and Thr and for residues incorporated after THP and Thr (2Nal and N). Fmoc deprotection was carried out using 20% piperidine in DMF (10 mL) under microwave conditions at 90° C., 1 min. Microcleavage of the resin with TFA shows the desired product. LCMS anal. calc. for C₉₄H₁₃₃N₂₁O₂₃S₂ 1989.349; found: 995.5 (M+2)²⁺.

Step B—Synthesis of Intermediate 2—To the resin intermediate 1 (0.25 mmol) was added a solution of 2-NITROBENZENESULFONYL CHLORIDE (221.6 mg, 1 mmol) and 2,4,6-TRIMETHYLPYRIDINE (330.4 μL, 0.917 g/mL, 2.5 mmol) in NMP (20 mL). The resin was mixed at room temperature for 50 mins. The resin was drained, washed with DMF (3×) and DCM (3×). Microcleavage of the resin shows the formation of the desired product. LCMS anal. calc. for C₁₀₀H₁₃₆N₂₂O₂₇S₃ 2174.509; found: 1087.8 (M+2)²⁺

Step C—Synthesis of Intermediate 3—The resin intermediate 2 (0.5 mmol) was swelled in DMF (50 mL) for 10 min. To this mixture was added IODINE (636 mg, 2.5 mmol) in DMF (10 mL) slowly, another 2 mL of DMF was used to rinse the vial and added to the reaction vessel. The resin was mixed at room temperature for 0.5 hr. The resin was drained. The resin was then washed with DMF, saturated sodium ascorbate solution in DMF, DMF and DCM. The resin was dried to use for the next step. Microcleavage of the resin shows the formation of the desired product. LCMS anal. calc. for C₁₀₀H₁₃₄N₂₂O₂₇S₃ 2172.493; found: 1086.8 (M+2)²⁺

Step D—Synthesis of Intermediate 4—The resin intermediate 3 (0.5 mmol) was swelled in THF (40 mL) for 15 min, then TBAF (1M in THF) (2.5 mL, 1 M, 2.5 mmol) was added. The reaction was mixed at room temperature for 1 hr. The resin was drained, washed with DMF (3×) and DCM (3×).

Step E—Synthesis of Intermediate 5—The resin intermediate 4 (0.32 mmol) was swelled DMF (30 mL) for 15 min, then heated to 50° C. A premixed solution of Iodine (812 mg, 3.2 mmol), TPP (1678 mg, 6.4 mmol) and imidazole (217.85 mg, 3.2 mmol) in DMF (13 mL) was added. The reaction was mixed at 50° C. for 30 mins, then drained, washed with DMF (3×) and DCM (3×). Microcleavage of the resin shows the formation of the desired product. LCMS anal. calc. for C₁₀₀H₁₃₃₁N₂₂O₂₆S₃ 2282.39; found: 1141.4 (M+2)²⁺

Step F—Synthesis of Intermediate 6—The resin intermediate 5 (0.32 mmol) was swelled in DMF (10 mL) for 15 min, then was added to a saturated CsCO₃ solution of DMF (80 mL) and the reaction mixture was heated at 60° C. for 1 hr. The resin is cooled down to room temperature, drained, washed with water (3×), DMF(3×) and DCM (3×). Microcleavage of the resin shows the formation of the desired product. LCMS anal. calc. for C₁₀₀H₁₃₂N₂₂O₂₆S₃ 2154.478; found: 1077.8 (M+2)²⁺

Step G—Synthesis of Int. 7 & Int. 8—Intermediate 6 (0.3 mmol) was swelled in DMF (12 mL) for 15 mins, then a solution of 1,8-DIAZABICYCLO[5.4.0]UNDEC-7-ENE (224.1 μL, 1.019 g/mL, 1.5 mmol) in DMF (3 mL) was added, followed by a solution of 2-MERCAPTOETHANOL (210.4 μL, 1.114 g/mL, 3 mmol) in DMF (3 mL). The reaction mixture was mixed for 20 min. The resin was washed with DCM and DMF. A fresh solution of B and C was added to the resin and mixed for another 20 min. The resin was washed with DMF, MeOH, and DCM and used for the next step. Microcleavage of the resin shows the formation of a mixture of the desired product Int. 7 and a side product Int.8. LCMS anal. calc. for C94H129N21022S2 & C₉₄H₁₃₁N₂₁O₂₂S₂ 1969.318, 1971.334 found: 985.0 & 986.0 (M+2)²⁺

Step H—Synthesis of Ex. 02—The mixture of Intermediate compound 7 &8 was treated with a cocktail solution of TFA/H₂O/TIPS 92.5/5/2.5 for 30 mins at 42° C. on a CEM Razor cleavage station. The mixture was then concentrated and then added to cold methyl-t-butyl ether in order to precipitate the peptide. After centrifugation, the peptide pellets were washed with fresh cold methyl-t-butyl ether to remove the organic scavengers. The process was repeated twice. The crude was then dissolved in 50% ACN/water (0.005 M). To this stirred solution was added IODINE (0.1 M) in MeOH drop-wise until it remained yellow. The reaction was stirred for 10 min then quenched with 1M ascorbic acid in water. Reactions were lyophilized and submitted for purification by preparative HPLC. The fractions containing products were combined and dried to give a white solid as title compound. LCMS anal. calc. for C₉₄H₁₂₉N₂₁O₂₂S₂ 1969.318; found: 985.0 (M+2)²⁺

### Example 2. Peptide Inhibition of Binding of Interleukin-23 to the Interleukin-23 Receptor

Peptide optimization was performed to identify peptide inhibitors of IL-23 signaling that were active at low concentrations (e.g., IC50<10 nM). Peptides were tested to identify peptides that inhibit the binding of IL-23 to human IL-23R and inhibit IL-23/IL-23R functional activity, as described below.

Assays were performed to determine peptide activity as described below, and the results of these assays are provided in Tables 3A-3H. Human ELISA indicates the IL23-IL23R competitive binding assay described below, Rat ELISA indicates the rat IL-23R competitive binding ELISA assay described below, and pStat3HTRF indicates the DB cells IL-23R pSTAT3 cell assay described below. The peptides depicted in Tables 3A-3H are cyclized via a disulfide bridge formed between two Pen residues in these peptides. The peptides depicted in Tables 3A-3H are cyclized via a thioether bond between the indicated amino acid residues. For certain peptides, the residue Abu is present where indicated, whereas in other embodiments, e.g., those related to the non-cyclized form, the Abu may be referred to as a hSer(C1) or homoSer residue.

### IL23-IL23R Competitive Binding ELISA

An Immulon® 4HBX plate was coated with 50 ng/well of IL23R_huFC and incubated overnight at 4° C. The wells were washed four times with PBST, blocked with PBS containing 3% Skim Milk for 1 hour at room temperature, and washed again four times with PBST. Serial dilutions of test peptides and IL-23 at a final concentration of 2 nM diluted in Assay Buffer (PBS containing 1% Skim Milk) were added to each well, and incubated for 2 hours at room temperature. After the wells were washed, bound IL-23 was detected by incubation with 50 ng/well of goat anti-p40 polyclonal antibodies (R&D Systems #AF309) diluted in Assay Buffer for 1 hour at room temperature. The wells were again washed four times with PBST. The secondary antibodies, HRP conjugated donkey anti-goat IgG (Jackson ImmunoResearch Laboratories #705-035-147) diluted 1:5000 in Assay Buffer was then added, and incubated for 30 minutes at room temperature. The plate was finally washed as above. Signals were visualized with TMB One Component HRP Membrane Substrate, quenched with 2 M sulfuric acid and read spectrophotometrically at 450 nm.

### Rat IL-23R Competitive Binding ELISA

An assay plate was coated with 300 ng/well of Rat IL-23R_huFC and incubated overnight at 4° C. The wells were washed, blocked, and washed again. Serial dilutions of test peptides and IL-23 at a final concentration of 7 nM were added to each well, and incubated for 2 hours at room temperature. After the wells were washed, bound IL-23 was detected with goat anti-p40 polyclonal antibodies, followed by an HRP conjugated donkey anti-goat IgG. Signals were visualized with TMB One Component HRP Membrane Substrate and quenched with 2 M sulfuric acid. IC50 values for various test peptides determined from these data are shown in Tables 3A-3H.

DB Cells IL23R pSTAT3 Cell Assay

IL-23 plays a central role in supporting and maintaining Th17 differentiation in vivo. This process is thought to mediated primarily through the Signal Transducer and Activator of Transcription 3 (STAT3), with phosphorylation of STAT3 (to yield pSTAT3) leading to upregulation of RORC and pro-inflammatory IL-17. This cell assay examines the levels of pSTAT3 in IL-23R-expressing DB cells when stimulated with IL-23 in the presence of test compounds. DB cells (ATCC #CRL-2289), cultured in RPMI-1640 medium (ATCC #30-2001) supplemented with 10% FBS and 1% Glutamine, were seeded at 5×10E5 cells/well in a 96 well tissue culture plate. Serial dilutions of test peptides and IL-23 at a final concentration of 0.5 nM were added to each well, and incubated for 30 minutes at 37° C. in a 5% CO₂ humidified incubator. Changes in phospho-STAT3 levels in the cell lysates were detected using the Cisbio HTRF pSTAT3 Cellular Assay Kit, according to manufacturer's Two Plate Assay protocol. IC50 values determined from these data are shown in Tables 3A-3H. Where not shown, data was not yet determined.

### Example 3. NK Cell Based Assay

Natural killer (NK) cells, purified from human peripheral blood of healthy donors by negative selection (Miltenyi Biotech, Cat #130-092-657), were cultured in complete media (RPMI 1640 containing 10% FBS, L-glutamine and penicillin-streptomycin) in the presence of IL-2 (RnD, Cat #202-IL-010/CF) at 25 ng/mL. After 7 days, cells were centrifuged, and resuspended in complete media at 1E6 cells/mL. Recombinant IL-23 at predetermined EC₅₀ to EC₇₅ and IL-18 (RnD, Cat #B003-5) at 10 ng/mL were mixed with varying concentrations of peptides, and added to NK cells seeded at 1E5 cells per well. After 20 to 24 hours, IFNγ in the supernatant was quantified using Quantikine ELISA (RnD, Cat #DIF50). IC₅₀ values determined from these data are shown. Where not shown, data was not yet determined.

### Example 4. IL-23R Reporter Assay

Compounds were serially diluted in 100% (v/v) DMSO) and plated using an Echo acoustic dispenser (Labcyte) into 1536-well non-treated black assay plates (Corning #9146). 3 μL of HEK293 cells containing IL-23R, IL-12Rβ1 and a firefly luciferase reporter gene driven by a STAT-inducible promoter (Promega) were added to the plates (4000 cells/well), followed by 3 μL of 20 ng/mL IL-23 (equivalent to EC₉₀ concentration). After 5 h at 37° C., 5% CO₂, 95% relative humidity, cells were placed at 20° C. and treated with BioGlo reagent (Promega) according to the manufacturer's instructions. Luminescence was measured on a Pherastar FSX (BMG LabTech). Data were normalized to IL-23 treatment (0% inhibition) and 30 μM of control inhibitor (100% inhibition), and IC₅₀ values were determined using a 4-parameter Hill equation. The data are shown in the tables that follow. Where multiple measurements have been made the average is shown with the number of replicates indicated in parenthesis following the IC₅₀ values.

TABLE 3

| Cmpd. No. | IC₅₀ (1 × 10−⁶ M (μM)) | Cmpd. No. | IC₅₀ (μM) |
|---|---|---|---|
| 201 | | 210 | 0.0022 |
| 202 | 7.15 | 211 | 0.0015 |
| 203 | 0.0088 | 212 | |
| 204 | 0.015 | 213 | .0096 |
| 205 | 0.0076 | 214 | 0.12 |
| 206 | 0.2 | 215 | .019 |
| 207 | 0.0029 | 216 | 0.25 |
| 208 | 0.003 | 217 | .031 |
| 209 | .0027 | | |

TABLE 4

| Cmpd. No. | IC₅₀ (μM) |
|---|---|
| 201 | 0.0034 |
| 209 | 0.0037 |
| 213 | 0.009 |
| 4 | 0.33 |
| 25 | 0.83 |
| 137 | 0.43 |
| 139 | >16.61 |
| 218 | >16.61 |
| 219 | >16.61 |
| 220 | >16.61 |
| 221 | ~9.41 |
| 222 | >16.61 |
| 223 | ~7.27 |
| 224 | 8.32 |
| 225 | >16.61 |
| 226 | 2.66 |
| 227 | 3.52 |
| 228 | >16.61 |
| 229 | 0.29 |
| 230 | 2.32 |
| 231 | 0.17 |
| 232 | >16.61 |
| 233 | >16.61 |
| 234 | 0.25 |
| 235 | >16.61 |
| 236 | >16.61 |
| 237 | 0.29 |
| 238 | >16.61 |
| 239 | >16.61 |
| 240 | 7.72 |
| 241 | >16.61 |
| 242 | >16.61 |
| 243 | 0.0021 |
| 244 | 0.0026 |
| 245 | 1.35 |
| 246 | 0.0054 |
| 247 | 3.56 |
| 248 | >16.61 |
| 249 | >16.61 |
| 250 | 0.0032 |
| 251 | 0.034 |
| 252 | 0.0028 |
| 253 | 0.27 |
| 254 | 0.29 |
| 255 | 0.006 |
| 256 | |
| 257 | 7.15 |
| 258 | 0.0088 |
| 259 | 0.015 |
| 260 | 0.0076 |
| 261 | 0.2 |
| 262 | 0.0029 |
| 263 | 0.003 |
| 264 | 0.0027 |
| 265 | 0.0022 |
| 266 | 0.0015 |
| 267 | |
| 268 | 0.0096 |
| 269 | 0.12 |
| 270 | 0.19 |
| 271 | 0.25 |
| 272 | 0.031 |
| 273 | 0.3 |
| 274 | 0.07 |
| 275 | 0.069 |
| 276 | 0.031 |
| 277 | 0.0029 |
| 278 | 0.0042 |
| 279 | 0.0024 |
| 280 | 0.0039 |
| 281 | 0.0038 |
| 282 | 0.0047 |
| 283 | 0.0049 |
| 284 | 0.0067 |
| 285 | 0.0049 |
| 286 | 0.0071 |
| 287 | 0.02 |
| 288 | 0.0066 |
| 289 | 0.0059 |
| 290 | 0.0046 |
| 291 | 0.009 |
| 292 | 0.0029 |
| 293 | |
| 294 | 0.0041 |
| 295 | 0.0044 |
| 296 | 0.0037 |
| 297 | 0.0028 |
| 298 | 0.001 |
| 299 | 0.0045 |
| 300 | 0.0045 |
| 301 | 0.0018 |
| 302 | 0.0028 |
| 303 | 0.0031 |
| 304 | 0.011 |
| 305 | 0.0018 |
| 306 | 0.0095 |
| 307 | 0.0018 |
| 308 | |
| 309 | |
| 310 | |
| 311 | |
| 312 | 0.036 |
| 313 | 0.016 |
| 314 | 0.021 |
| 315 | 0.029 |
| 316 | 0.039 |
| 317 | 0.017 |
| 318 | 0.0025 |
| 319 | 0.0028 |
| 320 | 0.0021 |
| 321 | 5.23 |
| 322 | 0.0013 |
| 323 | 0.0018 |
| 324 | 0.35 |
| 325 | 0.0016 |
| 326 | 0.0038 |
| 327 | 0.077 |
| 328 | 0.14 |

### PBMC PSTAT3 Assay

Cryopreserved peripheral blood mononuclear cells (PBMCs) from healthy donors were thawed and washed twice in ImmunoCult-XF T cell expansion medium (XF-TCEM) supplemented with CTL anti-aggregate wash. The cells were counted, resuspended at 2×105 cells per mL XF-TCEM supplemented with penicillin/streptomycin and 100 ng/mL IL-1$ (BioLegend, 579404), and cultured in tissue culture flasks coated with anti-CD3 (eBioscience, 16-0037-85 or BD Pharmingen, 555329) at 37° C. in 5% C02. On day 4 of culture, PBMCs were collected, washed twice in RPMI-1640 supplemented with 0.1% BSA (RPMI-BSA), and incubated in RPMI-BSA in upright tissue culture flasks for 4 hours at 37° C. in 5% C02. Following this ‘starvation,’ a total of 6×10⁴ cells in 30 μL RPMI-BSA was transferred into each well of a 384-well plate pre-spotted with peptide or DMSO. The cells were incubated for 30 minutes prior to the addition of IL-23 at a final concentration of 5 ng/mL. The cells were stimulated with cytokine for 30 minutes at 37° C. in 5% C02, transferred onto ice for 10 minutes, and lysed. Cell lysates were stored at −80° C. until phosphorylated STAT3 was measured using the phospho-STAT panel kit (Meso Scale Discovery, K15202D). The results produced with PBMCs are provide in Table 5 below for several from Examples along with data from the IL23R Reporter Assays utilizing HEK293 cells described above. The results are reported for single assays or as the average of replicate assays as indicated by the number in parentheses following the IC50 value.

TABLE 5

| Compd | PBMC pSTAT3 IC50 (nM) | SEQ ID NO: |
|---|---|---|
| 221 | 1100 | 166 |
| 226 | 96 | 171 |
| 288 | 0.004 | 233 |
| 202 | 0.0155 | 2 |
| 250 | 0.0277 | 195 |
| 289 | 0.0056 | 234 |
| 265 | 0.0020 | 210 |
| 327 | 0.40 | 272 |
| 320 | 0.0036 | 265 |
| 319 | 0.0079 | 264 |

Although the foregoing invention has been described in some detail by way of illustration and Example for purposes of clarity of understanding, one of skill in the art will appreciate that certain changes and modifications may be practiced within the scope of the appended claims. The full scope of the invention should be determined by reference to the claims, along with their full scope of equivalents, and the specification, along with such variations. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A bicyclic peptide inhibitor of an interleukin-23 receptor of Formula (I), comprising an amino acid sequence:
(I)
R1-X4-X5-T-X7-X8-X9-AEF-X11-X12-X13-N-X15-meG-R
wherein:
R1 is 7Ahp, 6Ahx, 5Ava, Peg2, AEEP, or AEEP(Ns);
X4 is Pen, Abu, aMeC, hC, or C;
X5 is N or K(PEG2PEG2gEC18OH);
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
X8 is KAc, Q, K(NMeAc), K(PEG2PEG2gEC18OH), dKAc, dQ, dK(NMeAc), or dK(PEG2PEG2gEC180H);
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is THP or aMeK;
X13 is E, dE, hE, D, dD, or hSer;
X15 is absent or 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, NmedY, N, dH, dN, dL, Aibor L;
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9, and
a second amide bond or thioether bond between R1 and X13; and
each alkyl of R2 optionally is substituted with Cl, F, or cyano.

2. A bicyclic peptide inhibitor of an interleukin-23 receptor of Formula (II), comprising an amino acid sequence of:
(II)
R1-X3-X4-X5-T-X7-K(Ac)-X9-AEF-X11-THP-X13-N-X15-
X16-R2
wherein:
R1 is Gaba, pFS, bAla or HOC16gEPEG2PEG2orn (also dOrn(HOC16gEPEG2PEG2));
X3 is dR, G, K(PEG2PEG2gEC18OH), R, dG, or dK(PEG2PEG2gEC18OH);
X4 is Pen, Abu, aMeC, hC, or C;
X5 is Nor Q;
X7 is 7MeW, or W
X9 is Pen, Abu, aMeC, hC, or C;

3. X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, dE, D, dD, or Dap(pF(6));
X15 is absent, 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, NMedY, N, dH, dN, dL, Aib, or L;
X16 is absent, meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, B, or dD; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9, and
a second bond between R1 and X13; and
each alkyl of R2 optionally optionally is substituted with Cl, F, or cyano.

4. A bicyclic peptide inhibitor of an interleukin-23 receptor of Formula (III′), comprising an amino acid sequence of:
(III′)
R1-X3-X4-X5-T-X7-X8-X9-X10-X11-X12-X13-N-X15-X16-
R2
wherein:
R1 is C₁ to C₄ alkyl C(O)—, or C₁ to C₄ alkyl C(O)— substituted with Cl, F, or cyano, 5Cpa, or cPEG3aCO;
X3 is R5H, R6H, R7H, S5H, S6H, S7H, K, dK, Orn, d-Orn, Dap, Dab(COCH2), dHe, or hK;
X4 is Pen, Abu, aMeC, hC, or C;
X5 is N, Q or N(N(Me)2), dN, dQ or dN(N(Me)2);
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or 7MedW; X8 is K(Ac), Q, K(NMeAc), dK(Ac), dQ, or dK(NMeAc);
X8 is KAc, Q, K(NMeAc), dK, dQ, dKAc, or dK(NMeAc);
X9 is Pen, Abu, aMeC, hC, or C;
X10 is AEF or TMAPF;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is THP or aMeK;
X13 is R5H, R6H, R7H, S5H, S6H, S7H, C, E, hE, KNMe, dC, dE, dhE, or dKNMe;
X15 is 3Pya;
X16 is meG;
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide, thioether, or aliphatic bond between X3 and X13.
each alkyl pf R2 optionally is substituted with Cl, F, or cyano.

5. A bicyclic peptide inhibitor of an interleukin-23 receptor of Formula (IV), comprising an amino acid sequence of:
(IV)
R1-X3-X4-X5-T-X7-X8-X9-AEF-X11-THP-X13-N-X15-X16-
R2
wherein:
R1 is C₁ to C₄ alkyl C(O)—, or C₁ to C₄ alkly C(O)— substituted with Cl, F, or cyano, 7Ahp, 6Ahx, or 5Ava;
X3 is absent, dR, R, dOrn, or Orn;
X4 is Pen, Abu, aMeC, hC, or C;
X5 is Q, dQ, dN, or N;
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or 7MedW;
X8 is KAc, Q, dKAc, or dQ;
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, aMeE, Aad, hE, K, dE, dAad, dhE, or dK;
X15 is absent, 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, or L;
X16 is absent, meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, B, or dD; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9;
a second amide bond between AEF and X13; and
each alkyl of R2 optionally is substituted with Cl, F, or cyano.

6. A bicyclic peptide inhibitor of an interleukin-23 receptor of Formula V, comprising an amino acid sequence:
(V)
R1-X4-N-T-X7-X8-X9-F4CONH2-X11-THP-X13-N-3Pya-
meG-R2
wherein:
R1 is C₁ to C₄ alkyl C(O)—, or C₁ to C₄ alkyl C(O)— substituted with Cl, F or cyano,
X4 is Pen, Abu, aMeC, hC or C;
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT or D7MeW;
X8 is K or dK;
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, dE, D, or dD; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by:
forming a first disulfide or thioether bond between X4 and X9;
a second amide bond between X8 and X13; and
each alkyl of R2 optionally is substituted with Cl, F, or cyano.

7. A bicyclic peptide inhibitor of an interleukin-23 receptor of Formula (VI), comprising an amino acid sequence:
(VI)
R1-X3-A-X5-T-X7-X8-A-AEF-X11-THP-X13-N-X15-R2
wherein:
R1 is C₁ to C₄ alkyl C(O)—, or C₁ to C₄ alkyl C(O)— substituted with Cl, F or cyano,
X3 is E, dE, D, or dD;
X5 is E, dE, D, or dD;
X7 is W or 7MeW;
X8 is KAc or dK(Ac);
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is KAc or dK(Ac);
X15 is absent 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming
a first amide bond between X5 and X10; and
a second amide bond between X3 and X15; and
each alkyl of R2 optionally is substituted with Cl, F or cyano.

8. A bicyclic peptide inhibitor of an interleukin-23 receptor of Formula (VII), comprising an amino acid sequence:
(VII)
R1-X3-X4-N-T-X7-K(Ac)-X9-X10-X11-THP-X13-N-3Pya-
X16-R2
where:
R1 is C₁ to C₄ alkyl C(O)—, or C₁ to C₄ alkyl C(O)— substituted with Cl, F or cyano,
X3 is D, dK, E, dDap, dD, K, dE, or Dap;
X4 is Pen, Abu, aMeC, hC, or C;
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
X9 is Pen, Abu, aMeC, hC, or C;
X10 is AEF, F4CONH2, or F40me;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is KAc or dKAc;
X16 is absent, meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, B, or dD;
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide bond between X3 and X16; and
each alkyl of R2 optionally is substituted with Cl, F or cyano.

9. A bicyclic peptide inhibitor of an interleukin-23 receptor of Formula (VIII), comprising an amino acid sequence:
(VIII)
R1-X4-X5-X6-X7-X8-X9-X10-X11-X12-X13-X14-3Pya-
meG-R2
wherein:
R1 is CF3CO, 5cpaCO, cPeg3aCO, C₁ to C₄ alkyl C(O)—, or C₁ to C₄ alkyl C(O)— substituted with cyano, Cl, F, or MeCo;
X4 is Pen, Abu, aMeC, hC, or C;
X5 is E, Dap, or K(NMe), dE, D, dD, or dK(NMe);
X6 is T, L, dT, dL, I, or dI;
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
X8 is KAc, KPeg12, KAcMor, Q(N(Me)2), K(Me)3, hK(Me)3; K(NMeAc), K(mPEG12), A, or Q, dKAc, dKPeg12, dKacMor, dQ(N(Me)2), KAc; kPeg12, KPeg12, KacMor, Q(N(Me)2), K(Me)3, hK(Me)3, K(NMeAc); K(mPEG12), dA, dQ, dhK(Me)3, dK(NMeAc), dK(mPEG12), dA, or dQ;
X9 is Pen, Abu, aMeC, hC, or C;
X10 is AEF or AEF(NMe);
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is THP, aMeLeu, or A;
X13 is KAc, A, L, K(NMeAc), Q(N(Me)2)), K(Me)3, E, dKAc, dA; dL, dK(NMeAc), dQ(N(Me)2)), dK(Me)3, or dE;
X14 is L, N, or S; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide or aliphatic (RCM) bond between between X5 and X10; and
each alkyl of R2 optionally is substituted with Cl, F, or cyano.

10. A bicyclic peptide inhibitor of an interleukin-23 receptor of Formula (IX), comprising an amino acid sequence:
(IX)
R1-X3-X4-X5-T-X7-X8-X9-X10-X11-THP-X13-X14-3Pya-
meG-R2
wherein:
R1 is C₁ to C₄ alkyl C(O)—, or C₁ to C₄ alkyl C(O)— substituted with Cl; F, or cyano, or HOC18gEPEG2PEG2CO;
X3 is R, dR, K, dK, dK(Me)3, K(Me)3, dK(PEG2PEG2gEC180H), or K(PEG2PEG2gEC180H);
X4 is Pen, Abu, aMeC, hC, or C;
X5 is E or dE;
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
X8 is KAc or dK(Ac);
X9 is Pen, Abu, aMeC, hC, or C;
X10 is AEF or AEF(NMe);
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is KAc, E, dK(Ac), or dE;
X14 is L, N, or S;
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide or aliphatic (RCM) bond between between X5 and X10; and
each alkyl of R2 optionally is substituted with Cl, F, or cyano.

11. A bicyclic peptide inhibitor of an interleukin-23 receptor of Formula (X), comprising an amino acid sequence:
(X)
X5-T-X7-X8-A-AEF-X11-THP-X13-3Pya
wherein:
X5 is E, dE, D, or dD;
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
X8 is KAc or dK(Ac);
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy; and
X13 is absent, KAc, or dK(Ac); and
wherein:
the bicyclic peptide inhibitor of an interleukin-23 receptor is cyclized by forming:
a first disulfide or thioether bond between X5 and AEF; and
a second cyclization between the amino terminus of X5 and the carboxy terminus of 3Pya.

12. A bicyclic peptide inhibitor of an interleukin-23 receptor of Formula (XI), comprising an amino acid sequence:
(XI)
R1-X4-X5-T-X7-X8-X9-AEF-X11-THP-X13-N-X15-R2
wherein:
R1 is 7Ahp, 6Ahx, 5Ava AEEP, or dK(PEG2PEG2gEC180H);
X4 is Pen, Abu, aMeC, hC, or C;
X5 is N, Q, dN, or dQ,
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
X8 is KAc, Q, dKAc, or dQ;
X9 is Pen, Abu, aMeC, hCor C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, dE, D, or dD;
X15 is absent, 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, or L;
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide or aliphatic (RCM) bond between between R1 and X13; and
each alkyl of R2 optionally is substituted with Cl, F, or cyano.

13. A bicyclic peptide inhibitor of an interleukin-23 receptor of Formula (XII), comprising an amino acid sequence:
(XII)
R1-X4-N-X6-X7-X8-X9-AEF-2Nal-X12-X13-N-3Pya-X16-
R2
wherein:
R1 is C₁ to C₄ alkyl C(O)—, or C₁ to C₄ alkyl C(O)— substituted with Cl, F, or cyano,
X4 is Pen, Abu, aMeC, hC, or C;
X6 is 3Hyp, T, 30HPro, or dT;
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW;
X8 is, R5H, R6H, R7H, S5H, S6H, or S7H;
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is, R5H, R6H, R7H, S5H, S6H, or S7H;
X16 is absent, meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, B, or dD; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide or aliphatic (RCM) bond between X3 and one of X10, X13, or X16.
each alkyl of R2 optionally is substituted with Cl, F, or cyano.

14. A bicyclic peptide inhibitor of an interleukin-23 receptor of Formula (XIII), comprising an amino acid sequence:
(XIII)
R1-X4-X5-T-X7-X8-X9-AEF-2Nal-THP-X13-N-X15-X16-
X17-R2
wherein:
R1 is 7Ahp, 6Ahx, 5Ava, AEEP, ordK(PEG2PEG2gEC180H);
X4 is Pen, Abu, aMeC, hC, or C;
X5 is N, Q, dN, or dQ,
X7 is W, 7MeW, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(40MePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, BT, or D7MeW; X8 is KAc, Q, dKAc, or dQ;
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, dE, D, or dD;
X15 is absent, 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, or L;
X16 is absent, meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD, or NMeK(PEG2PEG2gEC180H);
X17 is absent, K(PEG2PEG2gECI80H), or dK(PEG2PEG2gECI80H), and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide bond between R1 and X13;
each alkyl of R2 optionally is substituted with Cl, F, or cyano.

15. A bicyclic peptide inhibitor of an interleukin-23 receptor of Formula (XIV), comprising an amino acid sequence:
wherein:
```
(XIV)
R1-X3-X4-XS-X6-X7-X8-X9-AEF-X11-X12-X13-N-X15-X16-R2
                                                  |
                                                  R3
```
R1 is —H, C₁ to C₄ alkyl C(O)—, or C₁ to C₄ alkyl C(O)— substituted with Cl, F, or cyano,
X3 is dK, or K;
X4 is Pen, Abu, aMeC, hC, or C;
X5 is N, Q, or Dap,
X6 is T dK, or K;
X7 is W, 7MeW, dW, or d7MeW;
X8 is K(Ac), Q, dK(Ac), or dQ;
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is THP, or aMeL;
X13 is E, K(Ac), dE, E, D, dD, or dK(Ac);
X15 is 3Pya, 3MeH, H, F, hF, Y, dY, Y(CHF2), PAF, oAMPhe, F(CF3), dPaf, 3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, L, or absent;
X16 is meG, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD or is absent;
R2 is —NH₂, N(H)(C₁-C₄ alkyl), —HN(C₁-C₄ alkyl), —N(C₁-C₄ alkyl)₂, each alkly optionally substituted with Cl, F, or cyano; and
R3 is PEG4 (—HN[(CH2)20]4(CH2)2CO—), PEG4DA (—OC[(CH2)20]4(CH2)2CO—), or C6-C20 saturated or unsaturated dicarboxylic acid (e.g., 1,10-decanedioic acid, 1,12-dodecanedioic acid, 1,14-tetradecanedioic acid, or 1,16-hexadecanedioic);
wherein the bicyclic peptide inhibitor of an interleukin-23 receptor is cyclized by a first disulfide or thioether bond between X4 and X9, and a second amide bond between the R3 group apended to the AEF residue at X10 and
(i) a Dpr residue at X5,
(ii) a K or dK at X6, or
(iii) a K, dK, or E at X13.

16. A bicyclic peptide inhibitor of an interleukin-23 receptor, comprising an amino acid sequence of Formula (XV)
(XV)
R1-X4-X5-T-X7-X8-X9-AEF-X11-THP-X13-N-X15-R2
wherein:
R1 is C₁ to C₄ alkyl C(O)—, or C₁ to C₄ alkyl C(O)— substituted with Cl, F, or cyano;
X4 is Pen, Abu, aMeC, hC, or C;
X5 is E, dE, D, or dD;
X7 is W, 7meW, dW, or d7MeW;
X8 is KAc, Q, dKAc, or dQ;
X9 is Pen, Abu, aMeC, hC, or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, KAc, dE, D, dD, or dKAc;
X15 is absent, 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, NmedY, N, dH, dN, dL, Aib, or L; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide bond between AEF and X5; and
each alkyl of R2 optionally is substituted with Cl, F, or cyano.

17. A bicyclic peptide inhibitor of an interleukin-23 receptor of Formula (XVI), comprising an amino acid sequence:
(Formula XVI)
R1-X4-X5-T-X7-X8-X9-AEF-X11-THP-X13-N-X15-R2
wherein:
R1 is C₁ to C₄ alkyl C(O)—, or C₁ to C₄ alkyl C(O)— substituted with Cl, F, or cyano;
X4 is Pen, Abu, aMeC, hC, or C;
X5 is N, L, dN, or dL;
X7 is W, 7meW, dW, or d7MeW;
X8 is KAc or dKAc;
X9 is Pen, Abu, aMeC, hC, or C;
X10 is F4CONH₂, 4AmF, or dF4CONH₂;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, dK, dDap, K, Dap, ordE;
X15 is absent, 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, or L; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide bond between X13 and X15, or X13 and X16;
each alkyl of R2 optionally is substituted with Cl, F, or cyano.

18. A bicyclic peptide inhibitor of an interleukin-23 receptor of Formula (XVII), comprising an amino acid sequence:
(XVII)
R1-X3-X4-X5-T-X7-X8-X9-X10-X11-THP-X13-X14-X15-
X16-R2
wherein:
R1 is C₁ to C₄ alkyl C(O)—, or C₁ to C₄ alkyl C(O)— substituted with Cl, F, or cyano;
X3 is Orn, E, dOrn, or dE;
X4 is Pen, Abu, aMeC, hC, or C;
X5 is N or dN;
X7 is W, 7meW, dW, or d7MeW;
X8 is KAc or dKAc;
X9 is Pen, Abu, aMeC, hC, or C;
X10 is F4CONH2 or AEF;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is E, KAc, dKAc, or dE;
X14 is absent or N;
X15 is absent, 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH, THP, bAla, NMedY, K, dK, NMeY, N, dH, dN, dL, Aib, or L;
X16 is absent, 4(R)OHPro, 4(S)AminoPro, 4diFPro, 5(R)diMePro, aMeP, N(3AmBenzyl)Gly, N(Cyclohexyl)Gly, N(Isobutyl)Gly, P, dP, K, dK, E, dE, R, dR, D, dD, dDap, meG, Dap, or dMeG; and
R2 is —NH₂, N(H)(C₁-C₄ alkyl), —HN(C₁-C₄ alkyl), or —N(C₁-C₄ alkyl)₂;
wherein:
the bicyclic peptide inhibitor of an interleukin-23 receptor is cyclized by forming:
a first disulfide or thioether bond between X4 and X9; and
a second amide bond between X3 and one of X10, X13, or X16;
each alkyl of R2 optionally is substituted with Cl, F, or cyano.

19. A tricyclic peptide inhibitor of an interleukin-23 receptor of Formula (XVIII), comprising an amino acid sequence:
(XVIII)
R1-X3-X4-X5-T-X7-X8-X9-AEF-X11-THP-X13-N-3Pya-
meG-X17-R2
wherein:
R1 is C₁ to C₄ alkyl C(O)—, or C₁ to C₄ alkyl C(O)— substituted with Cl, F, or cyano,
X3 is K, dK, E, or dE;
X4 is Pen, Abu, aMeC, hC, or C;
X5 is E, dE, D, or dD;
X7 is W or 7MeW;
X8 is KAc or dK(Ac);
X9 is Pen, Abu, aMeC, hC or C;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X13 is KAc or dK(Ac);
X17 is E, dE, K, dK, D, or dD; and
R2 is —NH₂, N(H)(C₁-C₄ alkyl), —HN(C₁-C₄ alkyl), or —N(C₁-C₄ alkyl)₂;
wherein:
the tricyclic peptide inhibitor of an interleukin-23 receptor is cyclized by forming:
a first disulfide or thioether bond between X4 and X9; and
a second bond between X3 and X17, and
a third bond between X5 and AEF;
each alkyl of R2 optionally is substituted with Cl, F, or cyano.

20. A bicyclic peptide inhibitor of an interleukin-23 receptor of Formula (XIX) comprising an amino acid sequence:
(XIX)
R1-X3-X4-X5-T-X7-X8-X9-X10-X11-X12-X13-X14-X15-
X16-X17-R2
wherein:
R1 is 7Ahp, 6Ahx, 5Ava, Peg2, PEGNMe, AEEP, AEEP(Ns), Gaba, pFS, bAla, C₁ to C₄ alkyl C(O)—, or C₁ to C₄ alkyl C(O)— substituted with Cl, F, or cyano, 5cpaCO, or cPEG3aCO;
X3 is absent, dR, R, G, R5H, R6H, R7H, S5H, S6H, S7H, K, dK, Orn, dOrn, Dap, dDap, Dab, dDab, Dab(COCH2), dDab(COCH2), hE, dhE, hK, dhK, dSer(MePEG2), or Ser(MePEG2);
X4 is Pen, Abu, or C;
X5 is N, dN, Q, dQ, N(N(Me)2), or dN(N(Me)2);
X7 is W, dW, 7MeW, or d7MeW;
X8 is K(Ac), dK(Ac), Q, dQ, K(NMeAc), or dK(NMeAc);
X9 is Pen, Abu, or C;
X10 is AEF, TMAPF, or AEF(NHPEG3a);
X11 is 2Nal;
X12 is THP, Acpx, or aMeK;
X13 is E, dE, hE, dhE, aMeE, d-aMeE, D, dD, Aad, dAad, K, dK, hSer, dhSer, Dap(pF), R5H, R6H, R7H, S5H, S6H, S7H, C, dC, K(NMe), or dK(NMe);
X14 is absent or N;
X15 is 3Pal, H, dH, 3MeH, 3MedH, F, dF, aMeF, aMedF, THP, bAla, NMeTyr, NMedY, K or dK;
X16 is absent or meG;
X17 is absent or K(PEG2PEG2gEC180H); and
R2 is —NH₂, N(H)(C₁-C₄ alkyl), —HN(C₁-C₄ alkyl), or —N(C₁-C₄ alkyl)₂, and
wherein:
the bicyclic peptide inhibitor of an interleukin-23 receptor is cyclized by forming:
a first disulfide or thioether bond between X4 and X9; and
a second amide, aliphatic (RCM), alkyl amine, or thioether linkage between R1 and X13 or between X3 and X13; and
each alkyl of R2 optionally is substituted with Cl, F, or cyano;

21. A bicyclic peptide inhibitor of an interleukin-23 receptor of Formula (XX′), comprising an amino acid sequence:
(XX′)
R1-X3-X4-X5-X6-X7-X8-X9-X10-X11-X12-X13-X14-X15-
X16-R2
wherein:
R1 is CF3CO, 5cpaCO, cPEG3aCO, C1 to C4 alkyl C(O)—, or C1 to C4 alkyl C(O)— substituted with cyano, Cl, or F;
X3 is absent, R, dR, K, dK, K(Me)3, dK(Me)3, hK(Me)3, dhK(Me)3, K(d), or dK(d);
X4 is Pen, Abu, or C;
X5 is E, dE, D, dD, K, dK, K(a), K(Ac), K(cPEG3aCO), K(d), K(G), Dap, or K(NMe), dK(NMe), K(NNs), or dK(NNs);
X6 is selected from T, L, dT, dL, I, or dI;
X7 is W, 7MeW, 7PhW, dW, d7MeW, d7PhW, or 7(3NAcPh)W;
X8 is K(Ac), dK(Ac), hK(Me)3, dhK(Me)3, K(Me)3, dK(Me)3, K(NMeAc), dK(NMeAc), K(NMecPEG3a), Q(N(Me)2), KPeg12, dKPeg12, KAcMor, A, Q, dKacMor, dQ(N(Me)2), K(mPEG12), dA, dQ, or dK(mPEG12);
X9 is Pen, Abu, or C;
X10 is AEF or AEF(NMe);
X11 is 2Nal;
X12 is THP, aMeLeu, or A;
X13 is E, dE, K(Ac), dK(Ac), K(Me)3, dK(Me)3, K(NMeAc), dK(NMeAc), K(NMecPEG3a), Q(N(Me)2), dQ(N(Me)2), A, dA, L, or dL;
X14 is L, N, or S;
X15 is 3Pal, L, dL, or Aib
X16 is meG; and
R2 is —NH₂, N(H)(C₁-C₄ alklyl, —HN(C₁-C₄ alkyl), or —N(C₁-C₄ alkyl)₂; and
wherein:
the bicyclic peptide inhibitor of an interleukin-23 receptor is cyclized by forming:
a first disulfide or thioether bond between X4 and X9; and
a second amide or alkyl amine linkage between X5 and X10; and
each alkyl of R2 optionally is substituted with Cl, F, or cyano.

22. A compound or a pharmaceutically acceptable salt thereof which is a compound selected from Table 1A, Table 1B, Table 1C, Table 1D, Table 1E, Table 1F, Table 1G or Table 1H, respectively.

23. A pharmaceutical composition comprising:
the compound or pharmaceutically acceptable salt thereof according to any one of claims 1 to 21; and
a pharmaceutically acceptable carrier, excipient, or diluent.

24. The pharmaceutical composition of claim 22, further comprising an enteric coating.

25. The pharmaceutical composition of claim 23, wherein the enteric coating protects and releases the pharmaceutical composition within a patient or subject's lower gastrointestinal system.

26. A method for treating automimmune, inflammatory diseases or related disorders, comprising administering a therapeutically effective amount of:
the compound or pharmaceutically acceptable salt thereof according to any one of claims 1 to 21; or
the pharmaceutical composition of any one of claims 22 to 24
to a subject or patient in need thereof.

27. Use of a therapeutically effective amount of:
[a] the compound or pharmaceutically acceptable salt thereof according to any one of claims 1 to 21; or
[b] the pharmaceutical composition of any one of claims 22 to 24, in manufacture of a medicament for treating an automimmune, inflammatory diseases or related disorders.

28. The method for treating automimmune, inflammatory diseases or related disorders according to claim 25 or the use according to claim 26, wherein the automimmune, inflammatory diseases or related disorders is selected from multiple sclerosis, asthma, rheumatoid arthritis, inflammation of the gut, inflammatory bowel diseases (IBDs), juvenile IBD, adolescent IBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

29. The method for treating automimmune, inflammatory diseases or related disorders or the use according to claim 27, wherein the automimmune, inflammatory diseases or related disorders is selected from Inflammatory Bowel Disease (IBD), Ulcerative colitis (UC), Crohn's Disease (CD), psoriasis (PsO) or psoriatic arthritis (PsA).

30. A method for treating inflammatory disease is inflammatory bowel disease (IBD), Crohn's disease, ulcerative colitis, psoriasis or psoriatic arthritis, comprising administering a therapeutically effective amount of:
the compound or pharmaceutically acceptable salt thereof according to any one of claims 1 to 21; or
the pharmaceutical composition of any one of claims 22 to 24
to a subject or patient in need thereof.


SEQUENCE LISTING (286 sequences)

SEQ ID NO: 1 - protein, 13 aa, synthetic construct
  REGION 1..13: note = Modified peptide: 201
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XNTWKXXXXXNXG

SEQ ID NO: 2 - protein, 13 aa, synthetic construct
  REGION 1..13: note = Modified peptide: 202
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XNTWKXXXXXNXG

SEQ ID NO: 3 - protein, 13 aa, synthetic construct
  REGION 1..13: note = Modified peptide: 203
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWQCXXXXNXG

SEQ ID NO: 4 - protein, 14 aa, synthetic construct
  REGION 1..14: note = Modified peptide: 204
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R6H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R6H (X13) to S5H (X3)14
XXNTWKXXXXXNXG

SEQ ID NO: 5 - protein, 13 aa, synthetic construct
  REGION 1..13: note = Modified peptide: 205
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: aMeK
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWQCXXKXNXG

SEQ ID NO: 6 - protein, 14 aa, synthetic construct
  REGION 1..14: note = Modified peptide: 206
  REGION 1..11: note = Disulfid - Disulfide bond
  REGION 3..7: note = Disulfid - Disulfide bond
  SITE 1: note = Mod_Res - C-terminal is: GABA (X13)
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond at R1: GABA (X13) to E (R1)14
RXNTWKXXXXENXG

SEQ ID NO: 7 - protein, 14 aa, synthetic construct
  REGION 1..14: note = Modified peptide: 207
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to S5H (X3)14
XXNTWKXXXXXNXG

SEQ ID NO: 8 - protein, 14 aa, synthetic construct
  REGION 1..14: note = Modified peptide: 208
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to S5H (X3)14
XXNTWKXXXXXNXG

SEQ ID NO: 9 - protein, 12 aa, synthetic construct
  REGION 1..12: note = Modified peptide: 209
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)12
XNTWKXXXXXNX

SEQ ID NO: 10 - protein, 14 aa, synthetic construct
  REGION 1..14: note = Modified peptide: 210
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R7H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R7H (X13) to S5H (X3)14
XXNTWKXXXXXNXG

SEQ ID NO: 11 - protein, 14 aa, synthetic construct
  REGION 1..14: note = Modified peptide: 211
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R7H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R7H (X13) to S5H (X3)14
XXNTWKXXXXXNXG

SEQ ID NO: 12 - protein, 13 aa, synthetic construct
  REGION 1..13: note = Modified peptide: 212
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)
XNTWQCXXXXNXG

SEQ ID NO: 13 - protein, 13 aa, synthetic construct
  REGION 1..13: note = Modified peptide: 213
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3MeH
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XNTWKXXXXXNHG

SEQ ID NO: 14 - protein, 13 aa, synthetic construct
  REGION 1..13: note = Modified peptide: 214
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 4: note = misc_feature - Modified Amino Acid: 7MeW
  SITE 5: note = misc_feature - Modified Amino Acid: KAc
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3MeH
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWKCXXXXNHG

SEQ ID NO: 15 - protein, 14 aa, synthetic construct
  REGION 1..14: note = Modified peptide: 215
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to E14
XXQTWQXXXXENXG

SEQ ID NO: 16 - protein, 14 aa, synthetic construct
  REGION 1..14: note = Modified peptide: 216
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dK
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to S5H (X3)14
KXQTWQXXXXXNXG

SEQ ID NO: 17 - protein, 14 aa, synthetic construct
  REGION 1..14: note = Modified peptide: 217
  REGION 1..11: note = Disulfid - Disulfide bond
  REGION 3..7: note = Disulfid - Disulfide bond
  SITE 1: note = Mod_Res - C-terminal is: pFS (X13)
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: Dpr(pF(6)) (R1)
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond at R1: pFS (X13) to Dpr(pF(6)) (R1)14
GXNTWKXXXXXNXG

SEQ ID NO: 18 - protein, 13 aa, synthetic construct
  REGION 1..13: note = Modified peptide: 1
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dK
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: bAla
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: K to E
KXQTWQXXXXENA

SEQ ID NO: 19 - protein, 12 aa, synthetic construct
  REGION 1..12: note = Modified peptide: 2
  REGION 5..12: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: nFEtOH
  SITE 8: note = misc_feature - Modified Amino Acid: 2Nal
  SITE 9: note = misc_feature - Modified Amino Acid: THP
  SITE 12: note = misc_feature - Modified Amino Acid: bAla
  SITE 12: note = Mod_Res - N-terminal is: am
  REGION 1..6: note = Disulfid - Cyclic bond: Abu (X9) to C12
XQTWQCXXXKNA

SEQ ID NO: 20 - protein, 14 aa, synthetic construct
  REGION 1..14: note = Modified peptide: 3
  REGION 5..14: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dR
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 3..8: note = Disulfid - Cyclic bond at R1: E (X10) to AEF (X5)14
RXETWKXXXXKNXG

SEQ ID NO: 21 - protein, 13 aa, synthetic construct
  REGION 1..13: note = Modified peptide: 4
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKSXG

SEQ ID NO: 22 - protein, 13 aa, synthetic construct
  REGION 1..13: note = Modified peptide: 5
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKAXG

SEQ ID NO: 23 - protein, 13 aa, synthetic construct
  REGION 1..13: note = Modified peptide: 6
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXASXG

SEQ ID NO: 24 - protein, 13 aa, synthetic construct
  REGION 1..13: note = Modified peptide: 7
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXAKSXG

SEQ ID NO: 25 - protein, 13 aa, synthetic construct
  REGION 1..13: note = Modified peptide: 8
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXAXKSXG

SEQ ID NO: 26 - protein, 13 aa, synthetic construct
  REGION 1..13: note = Modified peptide: 9
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to K (X5)
XETWKXKXXKSXG

SEQ ID NO: 27 - protein, 13 aa, synthetic construct
  REGION 1..13: note = Modified peptide: 10
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWAXXXXKSXG

SEQ ID NO: 28 - protein, 13 aa, synthetic construct
  REGION 1..13: note = Modified peptide: 11
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XEAWKXXXXKSXG

SEQ ID NO: 29 - protein, 13 aa, synthetic construct
  REGION 1..13: note = Modified peptide: 12
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 5: note = misc_feature - Modified Amino Acid: KAc
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 3..8: note = Disulfid - Cyclic bond at R1: E (X10) to AEF (X5)
XETWKCXXXKSXG

SEQ ID NO: 30 - protein, 13 aa, synthetic construct
  REGION 1..13: note = Modified peptide: 13
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 5: note = misc_feature - Modified Amino Acid: KAc
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 3..8: note = Disulfid - Cyclic bond at R1: E (X10) to K (X5)
XETWKCKXXKSXG

SEQ ID NO: 31 - protein, 13 aa, synthetic construct
  REGION 1..13: note = Modified peptide: 14
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: K (X10) to E (X5)
XKTWKXEXXKSXG

SEQ ID NO: 32 - protein, 13 aa, synthetic construct
  REGION 1..13: note = Modified peptide: 15
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 5: note = misc_feature - Modified Amino Acid: KAc
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 3..8: note = Disulfid - Cyclic bond at R1: K (X10) to E (X5)
XKTWKCEXXKSXG

SEQ ID NO: 33 - protein, 10 aa, synthetic construct
  REGION 1..10: note = Modified peptide: 16
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 10: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)10
XETWKXXXXK

SEQ ID NO: 34 - protein, 9 aa, synthetic construct
  REGION 1..9: note = Modified peptide: 17
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 9: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)9
XETWKXXXX

SEQ ID NO: 35 - protein, 13 aa, synthetic construct
  REGION 1..13: note = Modified peptide: 18
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dOrn
  SITE 2: note = misc_feature - Modified Amino Acid: Pen
  SITE 5: note = misc_feature - Modified Amino Acid: 7meW
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: dOrn (X13) to E
XXNTWQXXXXENX

SEQ ID NO: 36 - protein, 14 aa, synthetic construct
  REGION 1..14: note = Modified peptide: 19
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dOrn
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 13: note = misc_feature - Modified Amino Acid: dK
  SITE 14: note = misc_feature - Modified Amino Acid: (D)NMeTyr
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: dOrn (X13) to E14
XXNTWKXXXXENKY

SEQ ID NO: 37 - protein, 13 aa, synthetic construct
  REGION 1..13: note = Modified peptide: 20
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dOrn
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: dOrn (X13) to E
XXNTWKXXXXENX

SEQ ID NO: 38 - protein, 13 aa, synthetic construct
  REGION 1..13: note = Modified peptide: 21
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XNTWKXXXXXNXG

SEQ ID NO: 39 - protein, 13 aa, synthetic construct
  REGION 1..13: note = Modified peptide: 22
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XNTWKXXXXXNXG

SEQ ID NO: 40 - protein, 12 aa, synthetic construct
  REGION 1..12: note = Modified peptide: 23
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 6Ahx (X13) to E (R1)12
XNTWKXXXXXNX

SEQ ID NO: 41 - protein, 12 aa, synthetic construct
  REGION 1..12: note = Modified peptide: 24
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 6Ahx (X13) to E (R1)12
XNTWKXXXXXNX

SEQ ID NO: 42 - protein, 12 aa, synthetic construct
  REGION 1..12: note = Modified peptide: 25
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: dNMeY
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)12
XNTWKXXXXXNY

SEQ ID NO: 43 - protein, 13 aa, synthetic construct
  REGION 1..13: note = Modified peptide: 26
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWQCXXXXNXG

SEQ ID NO: 44 - protein, 13 aa, synthetic construct
  REGION 1..13: note = Modified peptide: 27
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Orn
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: aMePhe
  SITE 13: note = Mod_Res - N-terminal is: am
  REGION 2..7: note = Disulfid - Cyclic bond: Abu (X9) to C
  REGION 1..11: note = Disulfid - Cyclic bond: Orn (X13) to E
XXQTWQCXXXENF

SEQ ID NO: 45 - protein, 13 aa, synthetic construct
  REGION 1..13: note = Modified peptide: 28
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: aMeK
  SITE 10: note = misc_feature - Modified Amino Acid: E (R1)
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWQCXXKXNFG

SEQ ID NO: 46 - protein, 12 aa, synthetic construct
  REGION 1..12: note = Modified peptide: 29
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)12
XNTWQCXXXXNX

SEQ ID NO: 47 - protein, 12 aa, synthetic construct
  REGION 1..12: note = Modified peptide: 30
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)12
XNTWKXXXXXNX

SEQ ID NO: 48 - protein, 12 aa, synthetic construct
  REGION 1..12: note = Modified peptide: 31
  REGION 4..10: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 3: note = misc_feature - Modified Amino Acid: 3Hyp
  SITE 4: note = misc_feature - Modified Amino Acid: 7meW
  SITE 5: note = misc_feature - Modified Amino Acid: R5H
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: R5H
  SITE 10: note = misc_feature - Modified Amino Acid: KAc
  SITE 12: note = misc_feature - Modified Amino Acid: 3Pya
  SITE 12: note = Mod_Res - N-terminal is: am
  REGION 1..6: note = Disulfid - Cyclic bond: Pen (X9) to Pen (X4)12
XNXWXXXXXKNX

SEQ ID NO: 49 - protein, 13 aa, synthetic construct
  REGION 1..13: note = Modified peptide: 32
  REGION 4..10: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: R5H
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: R5H
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
XNTWXXXXXKNXG

SEQ ID NO: 50 - protein, 12 aa, synthetic construct
  REGION 1..12: note = Modified peptide: 33
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)12
XNTWKXXXXXNX

SEQ ID NO: 51 - protein, 13 aa, synthetic construct
  REGION 1..13: note = Modified peptide: 34
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF (R3)
  SITE 8: note = misc_feature - Modified Amino Acid: 3Quin
  SITE 9: note = misc_feature - Modified Amino Acid: THP
  SITE 13: note = misc_feature - Modified Amino Acid: MeGly
  SITE 13: note = Mod_Res - N-terminal is: amPEG4 (X10)
  REGION 1..6: note = Disulfid - Cyclic bond: Pen (X9) to Pen (X4)
XNTWKXXXXENHG

SEQ ID NO: 52 - protein, 14 aa, synthetic construct
  REGION 1..14: note = Modified peptide: 35
  REGION 8..11: note = Disulfid - Disulfide bond
  REGION 3..7: note = Disulfid - Disulfide bond
  SITE 1: note = Mod_Res - C-terminal is: Gaba
  SITE 1: note = misc_feature - Modified Amino Acid: dR
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 8..11: note = Disulfid - Cyclic bond: AEF to E14
RXNTWKXXXXENXG

SEQ ID NO: 53 - protein, 15 aa, synthetic construct
  REGION 1..15: note = Modified peptide: 36
  REGION 2..15: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dK
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: MeGly
  SITE 15: note = Mod_Res - N-terminal is: am
  REGION 2..7: note = Disulfid - Cyclic bond: Pen (X9) to Pen (X4)
  REGION 1..11: note = Disulfid - Cyclic bond: K to K15
KXETWKXXXXKNXGE

SEQ ID NO: 54 - protein, 15 aa, synthetic construct
  REGION 1..15: note = Modified peptide: 37
  REGION 2..15: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: MeGly
  SITE 15: note = misc_feature - Modified Amino Acid: dK
  SITE 15: note = Mod_Res - N-terminal is: am
  REGION 2..7: note = Disulfid - Cyclic bond: Pen (X9) to Pen (X4)
  REGION 1..11: note = Disulfid - Cyclic bond: E to K15
EXETWKXXXXKNXGK

SEQ ID NO: 55 - protein, 14 aa, synthetic construct
  REGION 1..14: note = Modified peptide: 38
  SITE 1: note = Mod_Res - C-terminal is: ac
  SITE 5: note = misc_feature - Modified Amino Acid: 7meW
  SITE 6: note = misc_feature - Modified Amino Acid: KAc
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 1..8: note = Disulfid - Cyclic bond: E to AEF
  REGION 1..14: note = Disulfid - Cyclic bond: k to K14
EAETWKAXXXKNXK

SEQ ID NO: 56 - protein, 10 aa, synthetic construct
  REGION 1..10: note = Modified peptide: 39
  REGION 4..10: note = Disulfid - Disulfide bond
  REGION 4..8: note = Disulfid - Disulfide bond
  SITE 1: note = Mod_Res - C-terminal is:
  SITE 3: note = misc_feature - Modified Amino Acid: 7meW
  SITE 4: note = misc_feature - Modified Amino Acid: KAc
  SITE 6: note = misc_feature - Modified Amino Acid: AEF
  SITE 7: note = misc_feature - Modified Amino Acid: 2Nal
  SITE 8: note = misc_feature - Modified Amino Acid: THP
  SITE 9: note = misc_feature - Modified Amino Acid: KAc
  SITE 10: note = misc_feature - Modified Amino Acid: 3Pal
  SITE 10: note = Mod_Res - N-terminal is:
  REGION 1..10: note = Disulfid - Cyclic bond: E to 3Pal
  REGION 1..10: note = Disulfid - Cyclic bond: E to AEF (X15)
  REGION 6..9: note = Disulfid - Cyclic bond at R1: AEF to 3PAL10
ETWKAXXXKX

SEQ ID NO: 57 - protein, 9 aa, synthetic construct
  REGION 1..9: note = Modified peptide: 40
  SITE 1: note = Mod_Res - C-terminal is:
  SITE 3: note = misc_feature - Modified Amino Acid: 7meW
  SITE 4: note = misc_feature - Modified Amino Acid: KAc
  SITE 6: note = misc_feature - Modified Amino Acid: AEF
  SITE 7: note = misc_feature - Modified Amino Acid: 2Nal
  SITE 8: note = misc_feature - Modified Amino Acid: THP
  SITE 9: note = misc_feature - Modified Amino Acid: 3Pal
  SITE 9: note = Mod_Res - N-terminal is:
  REGION 1..9: note = Disulfid - Cyclic bond: E to 3Pal
  REGION 1..9: note = Disulfid - Cyclic bond: E to AEF (X15)9
ETWKAXXXX

SEQ ID NO: 58 - protein, 11 aa, synthetic construct
  REGION 1..11: note = Modified peptide: 41
  SITE 1: note = Mod_Res - C-terminal is: ac
  SITE 1: note = misc_feature - Modified Amino Acid: Orn
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = Mod_Res - N-terminal is: am
  REGION 2..7: note = Disulfid - Cyclic bond: Pen (X9) to Pen (X4)
  REGION 1..11: note = Disulfid - Cyclic bond: Orn (X13) to E11
XXNTWKXXXXE

SEQ ID NO: 59 - protein, 13 aa, synthetic construct
  REGION 1..13: note = Modified peptide: 42
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
XDTWKXXXXKNHG

SEQ ID NO: 60 - protein, 13 aa, synthetic construct
  REGION 1..13: note = Modified peptide: 43
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
XETWKXXXXKNHG

SEQ ID NO: 61 - protein, 13 aa, synthetic construct
  REGION 1..13: note = Modified peptide: 44
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Dpr
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: bAla
  SITE 13: note = Mod_Res - N-terminal is: am
  REGION 2..7: note = Disulfid - Cyclic bond: Abu (X9) to C
  REGION 8..11: note = Disulfid - Cyclic bond: AEF to E (X10)
XXQTWQCXXXENA

SEQ ID NO: 62 - protein, 13 aa, synthetic construct
  REGION 1..13: note = Modified peptide: 45
  SITE 1: note = Mod_Res - C-terminal is: ac
  SITE 1: note = misc_feature - Modified Amino Acid: Pen
  SITE 2: note = misc_feature - Modified Amino Acid: Dpr
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF (R3)
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 13: note = misc_feature - Modified Amino Acid: MeGly
  SITE 13: note = Mod_Res - N-terminal is: amPEG4DA (X10)
  REGION 1..6: note = Disulfid - Cyclic bond: Pen (X9) to Pen (X4)
XXTWKXXXXKNHG

SEQ ID NO: 63 - protein, 13 aa, synthetic construct
  REGION 1..13: note = Modified peptide: 46
  SITE 1: note = Mod_Res - C-terminal is: ac
  SITE 1: note = misc_feature - Modified Amino Acid: Pen
  SITE 2: note = misc_feature - Modified Amino Acid: Dpr
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF (R3)
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 13: note = misc_feature - Modified Amino Acid: MeGly
  SITE 13: note = Mod_Res - N-terminal is: amPEG2DA (X10)
  REGION 1..6: note = Disulfid - Cyclic bond: Pen (X9) to Pen (X4)
XXTWKXXXXKNHG

SEQ ID NO: 64 - protein, 13 aa, synthetic construct
  REGION 1..13: note = Modified peptide: 47
  SITE 1: note = Mod_Res - C-terminal is: ac
  SITE 1: note = misc_feature - Modified Amino Acid: Pen
  SITE 2: note = misc_feature - Modified Amino Acid: Dpr
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF (R3)
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 13: note = misc_feature - Modified Amino Acid: MeGly
  SITE 13: note = Mod_Res - N-terminal is: amDecanedioic_Acid (X10)
  REGION 1..6: note = Disulfid - Cyclic bond: Pen (X9) to Pen (X4)
XXTWKXXXXKNHG

SEQ ID NO: 65 - protein, 12 aa, synthetic construct
  REGION 1..12: note = Modified peptide: 48
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = Mod_Res - N-terminal is: am
  REGION 1..6: note = Disulfid - Cyclic bond: Pen (X9) to Pen (X4)12
XNEWQXXXLKNN

SEQ ID NO: 66 - protein, 13 aa, synthetic construct
  REGION 1..13: note = Modified peptide: 49
  SITE 1: note = Mod_Res - C-terminal is: ac
  SITE 1: note = misc_feature - Modified Amino Acid: dK
  SITE 2: note = misc_feature - Modified Amino Acid: Pen
  SITE 7: note = misc_feature - Modified Amino Acid: Pen
  SITE 8: note = misc_feature - Modified Amino Acid: AEF (R3)
  SITE 9: note = misc_feature - Modified Amino Acid: 2Nal
  SITE 10: note = misc_feature - Modified Amino Acid: aMeLeu
  SITE 11: note = misc_feature - Modified Amino Acid: KAc
  SITE 13: note = Mod_Res - N-terminal is: amPEG4DA (X10)
  REGION 2..7: note = Disulfid - Cyclic bond: Pen (X9) to Pen (X4)
  REGION 1..11: note = Disulfid - Cyclic bond: K to K
KXNTWQXXXXKNN

SEQ ID NO: 67 - protein, 12 aa, synthetic construct
  REGION 1..12: note = Modified peptide: 50
  SITE 1: note = Mod_Res - C-terminal is: ac
  SITE 1: note = misc_feature - Modified Amino Acid: Pen
  SITE 6: note = misc_feature - Modified Amino Acid: Pen
  SITE 7: note = misc_feature - Modified Amino Acid: AEF (R3)
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = Mod_Res - N-terminal is: amPEG4DA (X10)
  REGION 1..6: note = Disulfid - Cyclic bond: Pen (X9) to Pen (X4)12
XNKWQXXXLKNN

SEQ ID NO: 68 - protein, 12 aa, synthetic construct
  REGION 1..12: note = Modified peptide: 51
  SITE 1: note = Mod_Res - C-terminal is: ac
  SITE 1: note = misc_feature - Modified Amino Acid: Pen
  SITE 6: note = misc_feature - Modified Amino Acid: Pen
  SITE 7: note = misc_feature - Modified Amino Acid: AEF (R3)
  SITE 8: note = misc_feature - Modified Amino Acid: 2Nal
  SITE 9: note = misc_feature - Modified Amino Acid: aMeLeu
  SITE 12: note = Mod_Res - N-terminal is: amPEG4DA (X10)
  REGION 1..6: note = Disulfid - Cyclic bond: Pen (X9) to Pen (X4)12
XNTWQXXXLKNN

SEQ ID NO: 69 - protein, 13 aa, synthetic construct
  REGION 1..13: note = Modified peptide: 52
  SITE 1: note = Mod_Res - C-terminal is: bAla (X13)
  SITE 1: note = misc_feature - Modified Amino Acid: dR
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond at R1: bAla (X13) to E (R1)
RXQTWQXXXXENX

SEQ ID NO: 70 - protein, 10 aa, synthetic construct
  REGION 1..10: note = Modified peptide: 53
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 10: note = Mod_Res - N-terminal is: am
  REGION 1..6: note = Disulfid - Cyclic bond: Abu (X9) to C
XQTWQCXXXX

SEQ ID NO: 71 - protein, 13 aa, synthetic construct
  REGION 1..13: note = Modified peptide: 54
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Orn
  SITE 2: note = misc_feature - Modified Amino Acid: Pen
  SITE 5: note = misc_feature - Modified Amino Acid: 7meW
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: Orn (X13) to E
XXNTWQXXXXENX

SEQ ID NO: 72 - protein, 13 aa, synthetic construct
  REGION 1..13: note = Modified peptide: 55
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 73 - protein, 13 aa, synthetic construct
  REGION 1..13: note = Modified peptide: 56
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: W-7Ph
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 74 - protein, 13 aa, synthetic construct
  REGION 1..13: note = Modified peptide: 57
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 2: note = misc_feature - Modified Amino Acid: Dpr
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
XXTWKXXXXKNXG

SEQ ID NO: 75 - protein, 12 aa, synthetic construct
  REGION 1..12: note = Modified peptide: 58
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: dL
  SITE 12: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)12
XETWKXXXXKNL

SEQ ID NO: 76 - protein, 12 aa, synthetic construct
  REGION 1..12: note = Modified peptide: 59
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: Aib
  SITE 12: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)12
XETWKXXXXKNX

SEQ ID NO: 77 - protein, 13 aa, synthetic construct
  REGION 1..13: note = Modified peptide: 60
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 4: note = misc_feature - Modified Amino Acid: W-7Ph
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 3..8: note = Disulfid - Cyclic bond at R1: E (X10) to AEF (X5)
XETWQCXXXKNXG

SEQ ID NO: 78 - protein, 13 aa, synthetic construct
  REGION 1..13: note = Modified peptide: 61
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 4: note = misc_feature - Modified Amino Acid: 7meW
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 3..8: note = Disulfid - Cyclic bond at R1: E (X10) to AEF (X5)
XETWQCXXXKNXG

SEQ ID NO: 79 - protein, 12 aa, synthetic construct
  REGION 1..12: note = Modified peptide: 62
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)12
XQTWQCXXXXNX

SEQ ID NO: 80 - protein, 13 aa, synthetic construct
  REGION 1..13: note = Modified peptide: 63
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKSXG

SEQ ID NO: 81 - protein, 12 aa, synthetic construct
  REGION 1..12: note = Modified peptide: 64
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Abuto C
  REGION 1..11: note = Disulfid - Cyclic bond at R1: 6Ahx to E (R1)12
XNTWQCXXXXNX

SEQ ID NO: 82 - protein, 12 aa, synthetic construct
  REGION 1..12: note = Modified peptide: 65
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: dL
  SITE 12: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)12
XETWKXXXXKNL

SEQ ID NO: 83 - protein, 11 aa, synthetic construct
  REGION 1..11: note = Modified peptide: 66
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 11: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)11
XETWKXXXXKN

SEQ ID NO: 84 - protein, 12 aa, synthetic construct
  REGION 1..12: note = Modified peptide: 67
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: dL
  SITE 12: note = Mod_Res - N-terminal is: am
  REGION 1..6: note = Disulfid - Cyclic bond: Abu (X9) to C
  REGION 3..8: note = Disulfid - Cyclic bond at R1: E (X10) to AEF (X5)12
XETWQCXXXKNL

SEQ ID NO: 85 - protein, 11 aa, synthetic construct
  REGION 1..11: note = Modified peptide: 68
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 4: note = misc_feature - Modified Amino Acid: 7meW
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 11: note = Mod_Res - N-terminal is: am
  REGION 1..6: note = Disulfid - Cyclic bond: Abu (X9) to C
  REGION 3..8: note = Disulfid - Cyclic bond at R1: E (X10) to AEF (X5)11
XETWQCXXXKN

SEQ ID NO: 86 - protein, 10 aa, synthetic construct
  REGION 1..10: note = Modified peptide: 69
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 4: note = misc_feature - Modified Amino Acid: 7meW
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 10: note = Mod_Res - N-terminal is: am
  REGION 1..6: note = Disulfid - Cyclic bond: Abu (X9) to C
  REGION 3..8: note = Disulfid - Cyclic bond at R1: E (X10) to AEF (X5)10
XETWQCXXXK

SEQ ID NO: 87 - protein, 9 aa, synthetic construct
  REGION 1..9: note = Modified peptide: 70
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 4: note = misc_feature - Modified Amino Acid: 7meW
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 9: note = Mod_Res - N-terminal is: am
  REGION 1..6: note = Disulfid - Cyclic bond: Abu (X9) to C
  REGION 3..8: note = Disulfid - Cyclic bond at R1: E (X10) to AEF (X5)9
XETWQCXXX

SEQ ID NO: 88 - protein, 12 aa, synthetic construct
  REGION 1..12: note = Modified peptide: 71
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: bAla
  SITE 12: note = Mod_Res - N-terminal is: am
  REGION 1..6: note = Disulfid - Cyclic bond: Abu (X9) to C
  REGION 8..11: note = Disulfid - Cyclic bond: AEF (X13) to E (X10)12
XQTWQCXXXXNA

SEQ ID NO: 89 - protein, 12 aa, synthetic construct
  REGION 1..12: note = Modified peptide: 72
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: aMeGlu
  SITE 12: note = misc_feature - Modified Amino Acid: bAla
  SITE 12: note = Mod_Res - N-terminal is: am
  REGION 1..6: note = Disulfid - Cyclic bond: Abu (X9) to C
  REGION 8..11: note = Disulfid - Cyclic bond: AEF (X13) to E (X10)12
XQTWQCXXXXNA

SEQ ID NO: 90 - protein, 12 aa, synthetic construct
  REGION 1..12: note = Modified peptide: 73
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: Aad
  SITE 12: note = misc_feature - Modified Amino Acid: bAla
  SITE 12: note = Mod_Res - N-terminal is: am
  REGION 1..6: note = Disulfid - Cyclic bond: Abu (X9) to C
  REGION 8..11: note = Disulfid - Cyclic bond: AEF (X13) to aMeGlu (X10)12
XQTWQCXXXXNA

SEQ ID NO: 91 - protein, 13 aa, synthetic construct
  REGION 1..13: note = Modified peptide: 74
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dR
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: bAla
  SITE 13: note = Mod_Res - N-terminal is: am
  REGION 2..7: note = Disulfid - Cyclic bond: Abu (X9) to C
  REGION 1..11: note = Disulfid - Cyclic bond: Dpr (X13) to E (X3)
RXQTWQCXXXENA

SEQ ID NO: 92 - protein, 12 aa, synthetic construct
  REGION 1..12: note = Modified peptide: 75
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: bAla
  SITE 12: note = Mod_Res - N-terminal is: am
  REGION 1..6: note = Disulfid - Cyclic bond: Pen (X9) to Pen (X4)
  REGION 8..11: note = Disulfid - Cyclic bond: AEF (X13) to Aad (X10)12
XQTWQXXXXXNA

SEQ ID NO: 93 - protein, 13 aa, synthetic construct
  REGION 1..13: note = Modified peptide: 76
  REGION 1..13: note = Disulfid - Disulfide bond
  REGION 4..8: note = Disulfid - Disulfide bond
  SITE 1: note = Mod_Res - C-terminal is: ac
  SITE 5: note = misc_feature - Modified Amino Acid: 7meW
  SITE 6: note = misc_feature - Modified Amino Acid: KAc
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: dK
  SITE 13: note = Mod_Res - N-terminal is: am
  REGION 1..8: note = Disulfid - Cyclic bond: E to AEF (X5)
  REGION 1..11: note = Disulfid - Cyclic bond: E to K
EAETWKAXXXKNK

SEQ ID NO: 94 - protein, 13 aa, synthetic construct
  REGION 1..13: note = Modified peptide: 77
  REGION 3..8: note = Disulfid - Disulfide bond
  REGION 3..7: note = Disulfid - Disulfide bond
  SITE 1: note = Mod_Res - C-terminal is: AcMorp
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 95 - protein, 13 aa, synthetic construct
  REGION 1..13: note = Modified peptide: 78
  REGION 3..8: note = Disulfid - Disulfide bond
  REGION 3..7: note = Disulfid - Disulfide bond
  SITE 1: note = Mod_Res - C-terminal is: AcMorp
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXLLNXG

SEQ ID NO: 96 - protein, 13 aa, synthetic construct
  REGION 1..13: note = Modified peptide: 79
  REGION 3..8: note = Disulfid - Disulfide bond
  REGION 3..7: note = Disulfid - Disulfide bond
  SITE 1: note = Mod_Res - C-terminal is: AcMorp
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKLXG

SEQ ID NO: 97 - protein, 14 aa, synthetic construct
  REGION 1..14: note = Modified peptide: 80
  REGION 1..14: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: D
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: D to K14
DXNTWKXXXXKNXK

SEQ ID NO: 98 - protein, 14 aa, synthetic construct
  REGION 1..14: note = Modified peptide: 81
  REGION 1..14: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dDpr
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: E to K14
EXNTWKXXXXKNXX

SEQ ID NO: 99 - protein, 14 aa, synthetic construct
  REGION 1..14: note = Modified peptide: 82
  REGION 1..8: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dDpr
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: E to K14
EXNTWKXXXXKNXX

SEQ ID NO: 100 - protein, 13 aa, synthetic construct
  REGION 1..13: note = Modified peptide: 83
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: dK
  SITE 13: note = Mod_Res - N-terminal is: am
  REGION 1..6: note = Disulfid - Cyclic bond: Pen (X9) to Pen (X4)
XNTWKXXXXKNXK

SEQ ID NO: 101 - protein, 13 aa, synthetic construct
  REGION 1..13: note = Modified peptide: 84
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: dD
  SITE 13: note = Mod_Res - N-terminal is: am
  REGION 1..6: note = Disulfid - Cyclic bond: Pen (X9) to Pen (X4)
XNTWKXXXXKNXD

SEQ ID NO: 102 - protein, 13 aa, synthetic construct
  REGION 1..13: note = Modified peptide: 85
  REGION 5..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: dE
  SITE 13: note = Mod_Res - N-terminal is: am
  REGION 1..6: note = Disulfid - Cyclic bond: Pen (X9) to Pen (X4)
XNTWKXXXXKNXE

SEQ ID NO: 103 - protein, 13 aa, synthetic construct
  REGION 1..13: note = Modified peptide: 86
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: dK
  SITE 13: note = Mod_Res - N-terminal is: am
  REGION 1..6: note = Disulfid - Cyclic bond: Pen to Pen
  REGION 11..13: note = Disulfid - Cyclic bond: E to dK
XNTWKXXXXXNXK

SEQ ID NO: 104 - protein, 13 aa, synthetic construct
  REGION 1..13: note = Modified peptide: 87
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: dD
  SITE 13: note = Mod_Res - N-terminal is: am
  REGION 1..6: note = Disulfid - Cyclic bond: Pen (X9) to Pen (X4)
  REGION 11..13: note = Disulfid - Cyclic bond: E (X16) to dD (X13)
XNTWKXXXXXNXD

SEQ ID NO: 105 - protein, 13 aa, synthetic construct
  REGION 1..13: note = Modified peptide: 88
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: dE
  SITE 13: note = Mod_Res - N-terminal is: am
  REGION 1..6: note = Disulfid - Cyclic bond: Pen (X9) to Pen (X4)
  REGION 11..13: note = Disulfid - Cyclic bond: E (X16) to dE(X13)
XNTWKXXXXXNXE

SEQ ID NO: 106 - protein, 13 aa, synthetic construct
  REGION 1..13: note = Modified peptide: 89
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 8..11: note = Disulfid - Cyclic bond:to E
XNTWKXXXXXNXG

SEQ ID NO: 107 - protein, 14 aa, synthetic construct
  REGION 1..14: note = Modified peptide: 90
  SITE 1: note = Mod_Res - C-terminal is: ac
  SITE 1: note = misc_feature - Modified Amino Acid: dK
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to K14
KXNTWKXXXXKNXK

SEQ ID NO: 108 - protein, 13 aa, synthetic construct
  REGION 1..13: note = Modified peptide: 91
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 8..11: note = Disulfid - Cyclic bond: K to E
XNTWKXXXXXNXG

SEQ ID NO: 109 - protein, 12 aa, synthetic construct
  REGION 1..12: note = Modified peptide: 92
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dK
  SITE 12: note = misc_feature - Modified Amino Acid: 3Pal
  REGION 1..6: note = Disulfid - Cyclic bond: Pen (X9) to Pen (X4)
  REGION 10..12: note = Disulfid - Cyclic bond: dK (X15) to 3Pal (X13)
XNTWKXXXXXNX

SEQ ID NO: 110 - protein, 12 aa, synthetic construct
  REGION 1..12: note = Modified peptide: 93
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dDpr
  SITE 12: note = misc_feature - Modified Amino Acid: 3Pal
  REGION 1..6: note = Disulfid - Cyclic bond: Pen (X9) to Pen (X4)12
XNTWKXXXXXNX

SEQ ID NO: 111 - protein, 13 aa, synthetic construct
  REGION 1..13: note = Modified peptide: 94
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dK
  SITE 12: note = misc_feature - Modified Amino Acid: 3Pya
  SITE 13: note = misc_feature - Modified Amino Acid: Aib
  REGION 1..6: note = Disulfid - Cyclic bond: Pen (X9) to Pen (X4)
  REGION 11..13: note = Disulfid - Cyclic bond: dK (X16) to Aib (X13)
XNTWKXXXXXNXX

SEQ ID NO: 112 - protein, 13 aa, synthetic construct
  REGION 1..13: note = Modified peptide: 95
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dDpr
  SITE 12: note = misc_feature - Modified Amino Acid: 3Pya
  SITE 13: note = misc_feature - Modified Amino Acid: Aib
  REGION 1..6: note = Disulfid - Cyclic bond: Pen (X9) to Pen (X4)
XNTWKXXXXXNXX

SEQ ID NO: 113 - protein, 13 aa, synthetic construct
  REGION 1..13: note = Modified peptide: 96
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dK
  SITE 12: note = misc_feature - Modified Amino Acid: 3Pya
  REGION 1..6: note = Disulfid - Cyclic bond: Pen (X9) to Pen (X4)
  REGION 8..11: note = Disulfid - Cyclic bond: dK (X16) to G (X13)
XNTWKXXXXXNXG

SEQ ID NO: 114 - protein, 13 aa, synthetic construct
  REGION 1..13: note = Modified peptide: 97
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dDpr
  SITE 12: note = misc_feature - Modified Amino Acid: 3Pya
  REGION 1..6: note = Disulfid - Cyclic bond: Pen (X9) to Pen (X4)
XNTWKXXXXXNXG

SEQ ID NO: 115 - protein, 13 aa, synthetic construct
  REGION 1..13: note = Modified peptide: 98
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dK
  SITE 12: note = misc_feature - Modified Amino Acid: 3Pya
  SITE 13: note = misc_feature - Modified Amino Acid: bAla
  REGION 1..6: note = Disulfid - Cyclic bond: Pen (X9) to Pen (X4)
  REGION 11..13: note = Disulfid - Cyclic bond: dK (X16) to bAla (X13)
XNTWKXXXXXNXA

SEQ ID NO: 116 - protein, 13 aa, synthetic construct
  REGION 1..13: note = Modified peptide: 99
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dDpr
  SITE 12: note = misc_feature - Modified Amino Acid: 3Pya
  SITE 13: note = misc_feature - Modified Amino Acid: bAla
  SITE 13: note = Mod_Res - N-terminal is:
  REGION 1..6: note = Disulfid - Cyclic bond: Pen (X9) to Pen (X4)
XNTWKXXXXXNXA

SEQ ID NO: 117 - protein, 13 aa, synthetic construct
  REGION 1..13: note = Modified peptide: 100
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4OMe
  SITE 8: note = misc_feature - Modified Amino Acid: 2Nal
  SITE 9: note = misc_feature - Modified Amino Acid: THP
  SITE 10: note = misc_feature - Modified Amino Acid: dK
  SITE 12: note = misc_feature - Modified Amino Acid: 3Pya
  SITE 13: note = misc_feature - Modified Amino Acid: meG
  REGION 1..6: note = Disulfid - Cyclic bond: Pen (X9) to Pen (X4)
  REGION 11..13: note = Disulfid - Cyclic bond: dK (X16) to meG (X13)
XLTWKXXXXXLXG

SEQ ID NO: 118 - protein, 14 aa, synthetic construct
  REGION 1..14: note = Modified peptide: 101
  SITE 1: note = Mod_Res - C-terminal is: ac
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: E to K14
EXNTWKXXXXKNXK

SEQ ID NO: 119 - protein, 13 aa, synthetic construct
  REGION 1..13: note = Modified peptide: 102
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: F4CONH2
  SITE 8: note = misc_feature - Modified Amino Acid: 2Nal
  SITE 9: note = misc_feature - Modified Amino Acid: THP
  SITE 10: note = misc_feature - Modified Amino Acid: dK
  SITE 12: note = misc_feature - Modified Amino Acid: 3Pya
  SITE 13: note = misc_feature - Modified Amino Acid: meG
  REGION 1..6: note = Disulfid - Cyclic bond: Pen (X9) to Pen (X4)
  REGION 11..13: note = Disulfid - Cyclic bond: dK (X13) to meG (X16)
XLTWKXXXXXNXG

SEQ ID NO: 120 - protein, 14 aa, synthetic construct
  REGION 1..14: note = Modified peptide: 103
  SITE 1: note = Mod_Res - C-terminal is: ac
  SITE 1: note = misc_feature - Modified Amino Acid: dK
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to K14
KXNTWKXXXXKNXK

SEQ ID NO: 121 - protein, 14 aa, synthetic construct
  REGION 1..14: note = Modified peptide: 104
  SITE 1: note = Mod_Res - C-terminal is: ac
  SITE 1: note = misc_feature - Modified Amino Acid: dK
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dE
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to K14
KXNTWKXXXXKNXE

SEQ ID NO: 122 - protein, 14 aa, synthetic construct
  REGION 1..14: note = Modified peptide: 105
  REGION 1..14: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dK
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dR
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to K14
KXNTWKXXXXKNXR

SEQ ID NO: 123 - protein, 14 aa, synthetic construct
  REGION 1..14: note = Modified peptide: 106
  REGION 1..14: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dDpr
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dE
  SITE 14: note = Mod_Res - N-terminal is: am
  REGION 2..7: note = Disulfid - Cyclic bond: Pen (X9) to Pen (X4)14
XXNTWKXXXXKNXE

SEQ ID NO: 124 - protein, 14 aa, synthetic construct
  REGION 1..14: note = Modified peptide: 107
  REGION 1..14: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dDpr
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dD
  SITE 14: note = Mod_Res - N-terminal is: am
  REGION 2..7: note = Disulfid - Cyclic bond: Pen (X9) to Pen (X4)14
XXNTWKXXXXKNXD

SEQ ID NO: 125 - protein, 14 aa, synthetic construct
  REGION 1..14: note = Modified peptide: 108
  REGION 1..14: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4CONH2
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: E to K14
EXNTWKXXXXKNXK

SEQ ID NO: 126 - protein, 13 aa, synthetic construct
  REGION 1..13: note = Modified peptide: 109
  REGION 3..8: note = Disulfid - Disulfide bond
  REGION 3..7: note = Disulfid - Disulfide bond
  SITE 1: note = Mod_Res - C-terminal is: PEG12_OMe
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWKXXXLLNXG

SEQ ID NO: 127 - protein, 13 aa, synthetic construct
  REGION 1..13: note = Modified peptide: 110
  REGION 3..8: note = Disulfid - Disulfide bond
  REGION 3..7: note = Disulfid - Disulfide bond
  SITE 1: note = Mod_Res - C-terminal is: PEG12_OMe
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXLKNXG

SEQ ID NO: 128 - protein, 13 aa, synthetic construct
  REGION 1..13: note = Modified peptide: 111
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXXKNXG

SEQ ID NO: 129 - protein, 13 aa, synthetic construct
  REGION 1..13: note = Modified peptide: 112
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXXKNXG

SEQ ID NO: 130 - protein, 13 aa, synthetic construct
  REGION 1..13: note = Modified peptide: 113
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWXXXXLLNXG

SEQ ID NO: 131 - protein, 13 aa, synthetic construct
  REGION 1..13: note = Modified peptide: 114
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWXXXXLLNXG

SEQ ID NO: 132 - protein, 13 aa, synthetic construct
  REGION 1..13: note = Modified peptide: 115
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWXXXXXLNXG

SEQ ID NO: 133 - protein, 13 aa, synthetic construct
  REGION 1..13: note = Modified peptide: 116
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWXXXXXLNXG

SEQ ID NO: 134 - protein, 13 aa, synthetic construct
  REGION 1..13: note = Modified peptide: 117
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLLNXG

SEQ ID NO: 135 - protein, 13 aa, synthetic construct
  REGION 1..13: note = Modified peptide: 118
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLKLXG

SEQ ID NO: 136 - protein, 13 aa, synthetic construct
  REGION 1..13: note = Modified peptide: 119
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXXKLXG

SEQ ID NO: 137 - protein, 13 aa, synthetic construct
  REGION 1..13: note = Modified peptide: 120
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLKNXG

SEQ ID NO: 138 - protein, 13 aa, synthetic construct
  REGION 1..13: note = Modified peptide: 121
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLLNXG

SEQ ID NO: 139 - protein, 13 aa, synthetic construct
  REGION 1..13: note = Modified peptide: 122
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLKLXG

SEQ ID NO: 140 - protein, 13 aa, synthetic construct
  REGION 1..13: note = Modified peptide: 123
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXXKLXG

SEQ ID NO: 141 - protein, 13 aa, synthetic construct
  REGION 1..13: note = Modified peptide: 124
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLKNXG

SEQ ID NO: 142 - protein, 13 aa, synthetic construct
  REGION 1..13: note = Modified peptide: 125
  REGION 3..8: note = Disulfid - Disulfide bond
  REGION 3..7: note = Disulfid - Disulfide bond
  SITE 1: note = Mod_Res - C-terminal is: PEG12_OMe
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 143 - protein, 13 aa, synthetic construct
  REGION 1..13: note = Modified peptide: 126
  REGION 3..8: note = Disulfid - Disulfide bond
  REGION 3..7: note = Disulfid - Disulfide bond
  SITE 1: note = Mod_Res - C-terminal is: PEG12_OMe
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWKXXXXLNXG

SEQ ID NO: 144 - protein, 13 aa, synthetic construct
  REGION 1..13: note = Modified peptide: 127
  REGION 3..8: note = Disulfid - Disulfide bond
  REGION 3..7: note = Disulfid - Disulfide bond
  SITE 1: note = Mod_Res - C-terminal is: PEG12_OMe
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXLLNXG

SEQ ID NO: 145 - protein, 13 aa, synthetic construct
  REGION 1..13: note = Modified peptide: 128
  REGION 3..8: note = Disulfid - Disulfide bond
  REGION 3..7: note = Disulfid - Disulfide bond
  SITE 1: note = Mod_Res - C-terminal is: PEG12_OMe
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXLKLXG

SEQ ID NO: 146 - protein, 13 aa, synthetic construct
  REGION 1..13: note = Modified peptide: 129
  REGION 3..8: note = Disulfid - Disulfide bond
  REGION 3..7: note = Disulfid - Disulfide bond
  SITE 1: note = Mod_Res - C-terminal is: PEG12_OMe
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKLXG

SEQ ID NO: 147 - protein, 13 aa, synthetic construct
  REGION 1..13: note = Modified peptide: 130
  REGION 3..8: note = Disulfid - Disulfide bond
  REGION 3..7: note = Disulfid - Disulfide bond
  SITE 1: note = Mod_Res - C-terminal is: AcMorp
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWKXXXLLNXG

SEQ ID NO: 148 - protein, 13 aa, synthetic construct
  REGION 1..13: note = Modified peptide: 131
  REGION 3..8: note = Disulfid - Disulfide bond
  REGION 3..7: note = Disulfid - Disulfide bond
  SITE 1: note = Mod_Res - C-terminal is: AcMorp
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XELWKXXXXLNXG

SEQ ID NO: 149 - protein, 13 aa, synthetic construct
  REGION 1..13: note = Modified peptide: 132
  REGION 3..8: note = Disulfid - Disulfide bond
  REGION 3..7: note = Disulfid - Disulfide bond
  SITE 1: note = Mod_Res - C-terminal is: AcMorp
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXLKLXG

SEQ ID NO: 150 - protein, 13 aa, synthetic construct
  REGION 1..13: note = Modified peptide: 133
  REGION 3..8: note = Disulfid - Disulfide bond
  REGION 3..7: note = Disulfid - Disulfide bond
  SITE 1: note = Mod_Res - C-terminal is: AcMorp
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXLKNXG

SEQ ID NO: 151 - protein, 14 aa, synthetic construct
  REGION 1..14: note = Modified peptide: 134
  REGION 1..8: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: E to K14
EXNTWKXXXXKNXG

SEQ ID NO: 152 - protein, 14 aa, synthetic construct
  REGION 1..14: note = Modified peptide: 135
  REGION 1..14: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7meW
  SITE 6: note = misc_feature - Modified Amino Acid: KAc
  SITE 7: note = misc_feature - Modified Amino Acid: Pen
  SITE 8: note = misc_feature - Modified Amino Acid: F4OMe
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: E to K14
EXNTWKXXXXKNXK

SEQ ID NO: 153 - protein, 14 aa, synthetic construct
  REGION 1..14: note = Modified peptide: 136
  REGION 1..8: note = Disulfid - Disulfide bond
  REGION 4..8: note = Disulfid - Disulfide bond
  SITE 1: note = Mod_Res - C-terminal is: ac
  SITE 1: note = misc_feature - Modified Amino Acid: dK
  SITE 5: note = misc_feature - Modified Amino Acid: 7meW
  SITE 6: note = misc_feature - Modified Amino Acid: KAc
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 1..8: note = Disulfid - Cyclic bond: K to AEF (X5)
  REGION 1..14: note = Disulfid - Cyclic bond: K to K14
KAETWKAXXXKNXK

SEQ ID NO: 154 - protein, 13 aa, synthetic construct
  REGION 1..13: note = Modified peptide: 137
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XNTWKXXXXXNXG

SEQ ID NO: 155 - protein, 12 aa, synthetic construct
  REGION 1..12: note = Modified peptide: 138
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: (D)NMeTyr
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)12
XNTWKXXXXXNY

SEQ ID NO: 156 - protein, 12 aa, synthetic construct
  REGION 1..12: note = Modified peptide: 139
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7meW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)12
XNTWKXXXXXNX

SEQ ID NO: 157 - protein, 13 aa, synthetic construct
  REGION 1..13: note = Modified peptide: 140
  REGION 1..13: note = Disulfid - Disulfide bond
  REGION 4..8: note = Disulfid - Disulfide bond
  SITE 1: note = Mod_Res - C-terminal is: ac
  SITE 5: note = misc_feature - Modified Amino Acid: 7meW
  SITE 6: note = misc_feature - Modified Amino Acid: KAc
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: dK
  SITE 13: note = Mod_Res - N-terminal is: am
  REGION 1..8: note = Disulfid - Cyclic bond: E to AEF (X5)
  REGION 1..11: note = Disulfid - Cyclic bond: E to K
EAETWKAXXXKNK

SEQ ID NO: 158 - protein, 13 aa, synthetic construct
  REGION 1..13: note = Modified peptide: 141
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXXKLXG

SEQ ID NO: 159 - protein, 13 aa, synthetic construct
  REGION 1..13: note = Modified peptide: 142
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: Kpeg12
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLKNXG

SEQ ID NO: 160 - protein, 13 aa, synthetic construct
  REGION 1..13: note = Modified peptide: 143
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLKLXG

SEQ ID NO: 161 - protein, 13 aa, synthetic construct
  REGION 1..13: note = Modified peptide: 144
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXXKLXG

SEQ ID NO: 162 - protein, 13 aa, synthetic construct
  REGION 1..13: note = Modified peptide: 145
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7meW
  SITE 5: note = misc_feature - Modified Amino Acid: KacMor
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: aMeLeu
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXLKNXG

SEQ ID NO: 163 - protein, 12 aa, synthetic construct
  REGION 1..12: note = Modified peptide: 51
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: bAla
  SITE 12: note = Mod_Res - N-terminal is: am
  REGION 1..6: note = Disulfid - Cyclic bond: Pen (X9) to Pen (X4)
  REGION 8..11: note = Disulfid - Cyclic bond: AEF (X13) to E (X10)12
XQTWQXXXXXNA

SEQ ID NO: 164 - protein, 12 aa, synthetic construct
  REGION 1..12: note = Modified peptide: 61
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: bAla
  SITE 12: note = Mod_Res - N-terminal is: am
  REGION 1..6: note = Disulfid - Cyclic bond: Abu (X9) to C
  REGION 8..11: note = Disulfid - Cyclic bond: AEF (X13) to E (X10)12
XQTWQCXXXXNA

SEQ ID NO: 165 - protein, 12 aa, synthetic construct
  REGION 1..12: note = Modified peptide: 77
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: aMeE
  SITE 12: note = misc_feature - Modified Amino Acid: bAla
  SITE 12: note = Mod_Res - N-terminal is: am
  REGION 1..6: note = Disulfid - Cyclic bond: Abu (X9) to C
  REGION 8..11: note = Disulfid - Cyclic bond: AEF (X13) to aMeE (X10)12
XQTWQCXXXXNA

SEQ ID NO: 166 - protein, 13 aa, synthetic construct
  REGION 1..13: note = Modified peptide: 78
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dK
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: bAla
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: K to E
KXQTWQXXXXENA

SEQ ID NO: 167 - protein, 12 aa, synthetic construct
  REGION 1..12: note = Modified peptide: 88
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: hE
  SITE 12: note = misc_feature - Modified Amino Acid: bAla
  SITE 12: note = Mod_Res - N-terminal is: am
  REGION 1..6: note = Disulfid - Cyclic bond: Abu (X9) to C
  REGION 8..11: note = Disulfid - Cyclic bond: AEF (X13) to hE (X10)12
XQTWQCXXXENA

SEQ ID NO: 168 - protein, 13 aa, synthetic construct
  REGION 1..13: note = Modified peptide: 100
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: R5H
  SITE 13: note = misc_feature - Modified Amino Acid: bAla
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to R5H (X3)
XXQTWQXXXXXNA

SEQ ID NO: 169 - protein, 13 aa, synthetic construct
  REGION 1..13: note = Modified peptide: 101
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: R5H
  SITE 13: note = misc_feature - Modified Amino Acid: bAla
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to R5H (X3)
XXQTWQXXXXXNA

SEQ ID NO: 170 - protein, 13 aa, synthetic construct
  REGION 1..13: note = Modified peptide: 116
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dR
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: bAla
  SITE 13: note = Mod_Res - N-terminal is: am
  REGION 2..7: note = Disulfid - Cyclic bond: Abu (X9) to C
  REGION 8..11: note = Disulfid - Cyclic bond: AEF (X13) to E (X10)
RXQTWQCXXXENA

SEQ ID NO: 171 - protein, 13 aa, synthetic construct
  REGION 1..13: note = Modified peptide: 117
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: S5H
  SITE 13: note = misc_feature - Modified Amino Acid: bAla
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to S5H (X3)
XXQTWQXXXXXNA

SEQ ID NO: 172 - protein, 13 aa, synthetic construct
  REGION 1..13: note = Modified peptide: 118
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Dpr
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: bAla
  SITE 13: note = Mod_Res - N-terminal is: am
  REGION 2..7: note = Disulfid - Cyclic bond: Abu (X9) to C
  REGION 8..11: note = Disulfid - Cyclic bond: AEF to E (X10)
XXQTWQCXXXENA

SEQ ID NO: 173 - protein, 12 aa, synthetic construct
  REGION 1..12: note = Modified peptide: 120
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: bAla
  SITE 12: note = Mod_Res - N-terminal is: am
  REGION 1..6: note = Disulfid - Cyclic bond: Abu (X9) to C
  REGION 8..11: note = Disulfid - Cyclic bond: AEF (X13) to K (X10)12
XQTWQCXXXKNA

SEQ ID NO: 174 - protein, 13 aa, synthetic construct
  REGION 1..13: note = Modified peptide: 127
  REGION 1..11: note = Disulfid - Disulfide bond
  REGION 3..7: note = Disulfid - Disulfide bond
  SITE 1: note = Mod_Res - C-terminal is: bAla (X13)
  SITE 1: note = misc_feature - Modified Amino Acid: dR
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: THP
  SITE 13: note = Mod_Res - N-terminal is: am
  REGION 2..7: note = Disulfid - Cyclic bond: Abu (X9) to C
  REGION 1..11: note = Disulfid - Cyclic bond at R1: bAla (X13) to E (R1)
RXQTWQCXXXENX

SEQ ID NO: 175 - protein, 10 aa, synthetic construct
  REGION 1..10: note = Modified peptide: 129
  REGION 1..8: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 10: note = Mod_Res - N-terminal is: am
  REGION 1..6: note = Disulfid - Cyclic bond: Abu (X9) to C
  REGION 1..10: note = Disulfid - Cyclic bond at R1: 6Ahx (X13) to E (R1)10
XQTWQCXXXX

SEQ ID NO: 176 - protein, 12 aa, synthetic construct
  REGION 1..12: note = Modified peptide: 140
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)12
XQTWQCXXXXNX

SEQ ID NO: 177 - protein, 9 aa, synthetic construct
  REGION 1..9: note = Modified peptide: 169
  REGION 1..9: note = Disulfid - Disulfide bond
  REGION 3..7: note = Disulfid - Disulfide bond
  SITE 1: note = Mod_Res - C-terminal is: 8Aoc (R2)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 9: note = Mod_Res - N-terminal is: COOH (R1)
  REGION 1..6: note = Disulfid - Cyclic bond: Abu (X9) to C9
XQTWQCXXX

SEQ ID NO: 178 - protein, 9 aa, synthetic construct
  REGION 1..9: note = Modified peptide: 170
  REGION 5..9: note = Disulfid - Disulfide bond
  REGION 3..7: note = Disulfid - Disulfide bond
  SITE 1: note = Mod_Res - C-terminal is: 8Aoc
  SITE 1: note = misc_feature - Modified Amino Acid: Abu
  SITE 7: note = misc_feature - Modified Amino Acid: AEF (R2)
  SITE 8: note = misc_feature - Modified Amino Acid: 2Nal
  SITE 9: note = misc_feature - Modified Amino Acid: THP
  SITE 9: note = Mod_Res - N-terminal is: COOH (X10)
  REGION 1..6: note = Disulfid - Cyclic bond: Abu (X9) to C9
XQTWQCXXX

SEQ ID NO: 179 - protein, 13 aa, synthetic construct
  REGION 1..13: note = Modified peptide: 205
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Orn
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: aMePhe
  SITE 13: note = Mod_Res - N-terminal is: am
  REGION 2..7: note = Disulfid - Cyclic bond: Abu (X9) to C
  REGION 1..11: note = Disulfid - Cyclic bond: Orn (X13) to E
XXQTWQCXXXENF

SEQ ID NO: 180 - protein, 13 aa, synthetic construct
  REGION 1..13: note = Modified peptide: 294
  REGION 1..11: note = Disulfid - Disulfide bond
  REGION 3..7: note = Disulfid - Disulfide bond
  SITE 1: note = Mod_Res - C-terminal is: bAla (X13)
  SITE 1: note = misc_feature - Modified Amino Acid: dR
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond at R1: bAla (X13) to E (R1)
RXQTWQXXXXENX

SEQ ID NO: 181 - protein, 13 aa, synthetic construct
  REGION 1..13: note = Modified peptide: 303
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Orn
  SITE 2: note = misc_feature - Modified Amino Acid: Pen
  SITE 5: note = misc_feature - Modified Amino Acid: 7MeW
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: Orn (X13) to E
XXNTWQXXXXENX

SEQ ID NO: 182 - protein, 12 aa, synthetic construct
  REGION 1..12: note = Modified peptide: 390
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)12
XNTWQCXXXXNX

SEQ ID NO: 183 - protein, 13 aa, synthetic construct
  REGION 1..13: note = Modified peptide: 429
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Orn
  SITE 2: note = misc_feature - Modified Amino Acid: Pen
  SITE 5: note = misc_feature - Modified Amino Acid: 7MeW
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: Orn (X13) to E
XXNTWQXXXXENX

SEQ ID NO: 184 - protein, 13 aa, synthetic construct
  REGION 1..13: note = Modified peptide: 430
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: d-Orn
  SITE 2: note = misc_feature - Modified Amino Acid: Pen
  SITE 5: note = misc_feature - Modified Amino Acid: 7MeW
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: AEF (X13) to E (X3)
XXNTWQXXXXENX

SEQ ID NO: 185 - protein, 14 aa, synthetic construct
  REGION 1..14: note = Modified peptide: 431
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Orn
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 13: note = misc_feature - Modified Amino Acid: dK
  SITE 14: note = misc_feature - Modified Amino Acid: NMeDTyr
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: Orn (X13) to E14
XXNTWKXXXXENKY

SEQ ID NO: 186 - protein, 13 aa, synthetic construct
  REGION 1..13: note = Modified peptide: 432
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Orn
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: Orn (X13) to E
XXNTWKXXXXENX

SEQ ID NO: 187 - protein, 13 aa, synthetic construct
  REGION 1..13: note = Modified peptide: 433
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: d-Orn
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 8..11: note = Disulfid - Cyclic bond: AEF (X13) to E (X10)
XXNTWKXXXXENX

SEQ ID NO: 188 - protein, 13 aa, synthetic construct
  REGION 1..13: note = Modified peptide: 448
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 189 - protein, 13 aa, synthetic construct
  REGION 1..13: note = Modified peptide: 451
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7PhW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 190 - protein, 13 aa, synthetic construct
  REGION 1..13: note = Modified peptide: 488
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: Acpx
  SITE 11: note = misc_feature - Modified Amino Acid: S5H
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to S5H (X3)
XXNTWKXXXXXNX

SEQ ID NO: 191 - protein, 13 aa, synthetic construct
  REGION 1..13: note = Modified peptide: 533
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XNTWKXXXXXNXG

SEQ ID NO: 192 - protein, 12 aa, synthetic construct
  REGION 1..12: note = Modified peptide: 539
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 6Ahx (X13) to E (R1)12
XNTWKXXXXXNX

SEQ ID NO: 193 - protein, 12 aa, synthetic construct
  REGION 1..12: note = Modified peptide: 540
  REGION 8..11: note = Disulfid - Disulfide bond
  REGION 3..7: note = Disulfid - Disulfide bond
  SITE 1: note = Mod_Res - C-terminal is: 6Ahx
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: THP
  SITE 12: note = Mod_Res - N-terminal is: am
  REGION 1..6: note = Disulfid - Cyclic bond: Pen (X9) to Pen (X4)
  REGION 8..11: note = Disulfid - Cyclic bond: AEF (X13) to E (X10)12
XNTWKXXXXXNX

SEQ ID NO: 194 - protein, 12 aa, synthetic construct
  REGION 1..12: note = Modified peptide: 542
  REGION 8..11: note = Disulfid - Disulfide bond
  REGION 3..7: note = Disulfid - Disulfide bond
  SITE 1: note = Mod_Res - C-terminal is: 7Ahp
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: NMeDTyr
  SITE 12: note = Mod_Res - N-terminal is: am
  REGION 1..6: note = Disulfid - Cyclic bond: Pen (X9) to Pen (X4)
  REGION 8..11: note = Disulfid - Cyclic bond: AEF (X13) to E (X10)12
XNTWKXXXXXNY

SEQ ID NO: 195 - protein, 13 aa, synthetic construct
  REGION 1..13: note = Modified peptide: 543
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWQCXXXXNXG

SEQ ID NO: 196 - protein, 14 aa, synthetic construct
  REGION 1..14: note = Modified peptide: 548
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R6H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R6H (X13) to S5H (X3)14
XXNTWKXXXXXNXG

SEQ ID NO: 197 - protein, 13 aa, synthetic construct
  REGION 1..13: note = Modified peptide: 562
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: aMeK
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWQCXXKXNXG

SEQ ID NO: 198 - protein, 12 aa, synthetic construct
  REGION 1..12: note = Modified peptide: 563
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)12
XNTWQCXXXXNX

SEQ ID NO: 199 - protein, 12 aa, synthetic construct
  REGION 1..12: note = Modified peptide: 564
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: THP
  SITE 12: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)12
XNTWKXXXXXNX

SEQ ID NO: 200 - protein, 14 aa, synthetic construct
  REGION 1..14: note = Modified peptide: 580
  REGION 1..11: note = Disulfid - Disulfide bond
  REGION 3..7: note = Disulfid - Disulfide bond
  SITE 1: note = Mod_Res - C-terminal is: GABA (X13)
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond at R1: GABA (X13) to E (R1)14
RXNTWKXXXXENXG

SEQ ID NO: 201 - protein, 12 aa, synthetic construct
  REGION 1..12: note = Modified peptide: 586
  REGION 4..10: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 3: note = misc_feature - Modified Amino Acid: 3Hyp
  SITE 4: note = misc_feature - Modified Amino Acid: 7MeW
  SITE 5: note = misc_feature - Modified Amino Acid: S5H
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: S5H
  SITE 10: note = misc_feature - Modified Amino Acid: KAc
  SITE 12: note = misc_feature - Modified Amino Acid: 3Pya
  SITE 12: note = Mod_Res - N-terminal is: am
  REGION 1..6: note = Disulfid - Cyclic bond: Pen (X9) to Pen (X4)12
XNXWXXXXXKNX

SEQ ID NO: 202 - protein, 12 aa, synthetic construct
  REGION 1..12: note = Modified peptide: 587
  REGION 4..10: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 3: note = misc_feature - Modified Amino Acid: 3Hyp
  SITE 4: note = misc_feature - Modified Amino Acid: 7MeW
  SITE 5: note = misc_feature - Modified Amino Acid: S5H
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: S5H
  SITE 10: note = misc_feature - Modified Amino Acid: KAc
  SITE 12: note = misc_feature - Modified Amino Acid: 3Pya
  SITE 12: note = Mod_Res - N-terminal is: am
  REGION 1..6: note = Disulfid - Cyclic bond: Pen (X9) to Pen (X4)12
XNXWXXXXXKNX

SEQ ID NO: 203 - protein, 14 aa, synthetic construct
  REGION 1..14: note = Modified peptide: 589
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to S5H (X3)14
XXNTWKXXXXXNXG

SEQ ID NO: 204 - protein, 14 aa, synthetic construct
  REGION 1..14: note = Modified peptide: 590
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: aMeK
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to S5H (X3)14
XXNTWKXXXXXNXG

SEQ ID NO: 205 - protein, 13 aa, synthetic construct
  REGION 1..13: note = Modified peptide: 591
  REGION 4..10: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: S5H
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: S5H
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
XNTWXXXXXKNXG

SEQ ID NO: 206 - protein, 14 aa, synthetic construct
  REGION 1..14: note = Modified peptide: 599
  REGION 8..11: note = Disulfid - Disulfide bond
  REGION 3..7: note = Disulfid - Disulfide bond
  SITE 1: note = Mod_Res - C-terminal is: GABA
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 8..11: note = Disulfid - Cyclic bond: AEF (X13) to E (X10)14
RXNTWKXXXXENXG

SEQ ID NO: 207 - protein, 14 aa, synthetic construct
  REGION 1..14: note = Modified peptide: 623
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 3..8: note = Disulfid - Cyclic bond at R1: E (X10) to AEF (X5)14
RXETWKXXXXKNXG

SEQ ID NO: 208 - protein, 14 aa, synthetic construct
  REGION 1..14: note = Modified peptide: 634
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R7H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R7H (X13) to S5H (X3)14
XXNTWKXXXXXNXG

SEQ ID NO: 209 - protein, 14 aa, synthetic construct
  REGION 1..14: note = Modified peptide: 640
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R7H
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R7H (X13) to S5H (X3)14
XXNTWKXXXXXNXG

SEQ ID NO: 210 - protein, 14 aa, synthetic construct
  REGION 1..14: note = Modified peptide: 653
  REGION 1..14: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Dbu(COCH2)
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Abu (X9) to C
  REGION 1..11: note = Disulfid - Cyclic bond: Dbu(COCH2) to C14
XXNTWQCXXXCNXG

SEQ ID NO: 211 - protein, 13 aa, synthetic construct
  REGION 1..13: note = Modified peptide: 654
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)
XNTWQCXXXXNXG

SEQ ID NO: 212 - protein, 13 aa, synthetic construct
  REGION 1..13: note = Modified peptide: 655
  REGION 8..11: note = Disulfid - Disulfide bond
  REGION 3..7: note = Disulfid - Disulfide bond
  SITE 1: note = Mod_Res - C-terminal is: 5Ava
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 8..11: note = Disulfid - Cyclic bond: AEF (X13) to E (X10)
XNTWQCXXXXNXG

SEQ ID NO: 213 - protein, 13 aa, synthetic construct
  REGION 1..13: note = Modified peptide: 686
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 4: note = misc_feature - Modified Amino Acid: 7MeW
  SITE 5: note = misc_feature - Modified Amino Acid: KAc
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3MeH
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWKCXXXXNHG

SEQ ID NO: 214 - protein, 14 aa, synthetic construct
  REGION 1..14: note = Modified peptide: 778
  REGION 1..11: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to E14
KXQTWQXXXXENXG

SEQ ID NO: 215 - protein, 13 aa, synthetic construct
  REGION 1..13: note = Modified peptide: 823
  REGION 1..11: note = Disulfid - Disulfide bond
  REGION 3..7: note = Disulfid - Disulfide bond
  SITE 1: note = Mod_Res - C-terminal is: 7Ahp (X13)
  SITE 1: note = misc_feature - Modified Amino Acid: Pen
  SITE 2: note = misc_feature - Modified Amino Acid: K(PEG2PEG2gEC18OH)
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: 7Ahp (X13) to E (R1)
XXTWKXXXXXNXG

SEQ ID NO: 216 - protein, 14 aa, synthetic construct
  REGION 1..14: note = Modified peptide: 824
  REGION 1..11: note = Disulfid - Disulfide bond
  REGION 3..7: note = Disulfid - Disulfide bond
  SITE 1: note = Mod_Res - C-terminal is: GABA (X13)
  SITE 1: note = misc_feature - Modified Amino Acid: dK(PEG2PEG2gEC18OH)
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond at R1: GABA (X13) to E (R1)14
XXNTWKXXXXENXG

SEQ ID NO: 217 - protein, 14 aa, synthetic construct
  REGION 1..14: note = Modified peptide: 826
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: R5H
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: S5H
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: R5H (X13) to S5H (X3)14
XXQTWQXXXXXNXG

SEQ ID NO: 218 - protein, 13 aa, synthetic construct
  REGION 1..13: note = Modified peptide: 850
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: NMeK(PEG2PEG2gEC18OH)
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XNTWQCXXXXNXX

SEQ ID NO: 219 - protein, 13 aa, synthetic construct
  REGION 1..13: note = Modified peptide: 851
  REGION 1..11: note = Disulfid - Disulfide bond
  REGION 3..7: note = Disulfid - Disulfide bond
  SITE 1: note = Mod_Res - C-terminal is: 6Ahx (X13)
  SITE 1: note = misc_feature - Modified Amino Acid: Abu
  SITE 2: note = misc_feature - Modified Amino Acid: K(PEG2PEG2gEC18OH)
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 6Ahx (X13) to E (R1)
XXTWQCXXXXNXG

SEQ ID NO: 220 - protein, 13 aa, synthetic construct
  REGION 1..13: note = Modified peptide: 852
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 2: note = misc_feature - Modified Amino Acid: K(PEG2PEG2gEC18OH)
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)
XXTWQCXXXXNXG

SEQ ID NO: 221 - protein, 14 aa, synthetic construct
  REGION 1..14: note = Modified peptide: 862
  REGION 1..11: note = Disulfid - Disulfide bond
  REGION 3..7: note = Disulfid - Disulfide bond
  SITE 1: note = Mod_Res - C-terminal is: HOC16gEPEG2PEG2orn (X13)
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond at R1: HOC16gEPEG2PEG2orn (X13) to E (R1)14
RXNTWKXXXXENXG

SEQ ID NO: 222 - protein, 13 aa, synthetic construct
  REGION 1..13: note = Modified peptide: 945
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: K(NMeAc)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: K(NMeAc)
  SITE 12: note = misc_feature - Modified Amino Acid: 3Pya
  SITE 13: note = misc_feature - Modified Amino Acid: MeGly
  SITE 13: note = Mod_Res - N-terminal is: CONHMe
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 223 - protein, 13 aa, synthetic construct
  REGION 1..13: note = Modified peptide: 946
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: K(NMeAc)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: K(NMeAc)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 224 - protein, 13 aa, synthetic construct
  REGION 1..13: note = Modified peptide: 950
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWKXXXXKNXG

SEQ ID NO: 225 - protein, 13 aa, synthetic construct
  REGION 1..13: note = Modified peptide: 956
  REGION 3..8: note = Disulfid - Disulfide bond
  REGION 3..7: note = Disulfid - Disulfide bond
  SITE 1: note = Mod_Res - C-terminal is: CF3CO
  SITE 1: note = misc_feature - Modified Amino Acid: Pen
  SITE 4: note = misc_feature - Modified Amino Acid: 7MeW
  SITE 5: note = misc_feature - Modified Amino Acid: Q(N(Me)2)
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 10: note = misc_feature - Modified Amino Acid: Q(N(Me)2)
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWQXXXXQNXG

SEQ ID NO: 226 - protein, 13 aa, synthetic construct
  REGION 1..13: note = Modified peptide: 957
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: Q(N(Me)2)
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 10: note = misc_feature - Modified Amino Acid: Q(N(Me)2)
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWQXXXXQNXG

SEQ ID NO: 227 - protein, 13 aa, synthetic construct
  REGION 1..13: note = Modified peptide: 980
  REGION 3..8: note = Disulfid - Disulfide bond
  REGION 3..7: note = Disulfid - Disulfide bond
  SITE 1: note = Mod_Res - C-terminal is: CF3CO
  SITE 1: note = misc_feature - Modified Amino Acid: Pen
  SITE 4: note = misc_feature - Modified Amino Acid: 7MeW
  SITE 5: note = misc_feature - Modified Amino Acid: K(NMeAc)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: K(NMeAc)
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 228 - protein, 13 aa, synthetic construct
  REGION 1..13: note = Modified peptide: 981
  REGION 3..8: note = Disulfid - Disulfide bond
  REGION 3..7: note = Disulfid - Disulfide bond
  SITE 1: note = Mod_Res - C-terminal is: CF3CO
  SITE 1: note = misc_feature - Modified Amino Acid: Pen
  SITE 4: note = misc_feature - Modified Amino Acid: 7MeW
  SITE 5: note = misc_feature - Modified Amino Acid: Q(N(Me)2)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: Q(N(Me)2)
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF (X5)
XETWQXXXXQNXG

SEQ ID NO: 229 - protein, 13 aa, synthetic construct
  REGION 1..13: note = Modified peptide: 1000
  REGION 3..8: note = Disulfid - Disulfide bond
  REGION 3..7: note = Disulfid - Disulfide bond
  SITE 1: note = Mod_Res - C-terminal is: CF3CO
  SITE 1: note = misc_feature - Modified Amino Acid: Pen
  SITE 4: note = misc_feature - Modified Amino Acid: 7MeW
  SITE 5: note = misc_feature - Modified Amino Acid: K(Me)3
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: K(NMeAc)
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 230 - protein, 13 aa, synthetic construct
  REGION 1..13: note = Modified peptide: 1001
  REGION 3..8: note = Disulfid - Disulfide bond
  REGION 3..7: note = Disulfid - Disulfide bond
  SITE 1: note = Mod_Res - C-terminal is: CF3CO
  SITE 1: note = misc_feature - Modified Amino Acid: Pen
  SITE 4: note = misc_feature - Modified Amino Acid: 7MeW
  SITE 5: note = misc_feature - Modified Amino Acid: K(Me)3
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: K(Me)3
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 231 - protein, 13 aa, synthetic construct
  REGION 1..13: note = Modified peptide: 1005
  REGION 3..8: note = Disulfid - Disulfide bond
  REGION 3..7: note = Disulfid - Disulfide bond
  SITE 1: note = Mod_Res - C-terminal is: CF3CO
  SITE 1: note = misc_feature - Modified Amino Acid: Pen
  SITE 4: note = misc_feature - Modified Amino Acid: 7MeW
  SITE 5: note = misc_feature - Modified Amino Acid: K(Me)3
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 10: note = misc_feature - Modified Amino Acid: K(Me)3
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWKXXXXKNXG

SEQ ID NO: 232 - protein, 14 aa, synthetic construct
  REGION 1..14: note = Modified peptide: 1043
  REGION 1..11: note = Disulfid - Disulfide bond
  REGION 3..7: note = Disulfid - Disulfide bond
  SITE 1: note = Mod_Res - C-terminal is: pFS (X13)
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: Dpr(pF(6)) (R1)
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond at R1: pFS (X13) to Dpr(pF(6)) (R1)14
GXNTWKXXXXXNXG

SEQ ID NO: 233 - protein, 13 aa, synthetic construct
  REGION 1..13: note = Modified peptide: 1044
  REGION 1..11: note = Disulfid - Disulfide bond
  REGION 3..7: note = Disulfid - Disulfide bond
  SITE 1: note = Mod_Res - C-terminal is: PEG2 (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: hE (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: PEG2 (X13) to hE (R1)
XNTWKXXXXXNXG

SEQ ID NO: 234 - protein, 14 aa, synthetic construct
  REGION 1..14: note = Modified peptide: 1055
  REGION 1..8: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: E to K14
EXNTWKXXXXKNXG

SEQ ID NO: 235 - protein, 13 aa, synthetic construct
  REGION 1..13: note = Modified peptide: 1067
  REGION 3..8: note = Disulfid - Disulfide bond
  REGION 3..7: note = Disulfid - Disulfide bond
  SITE 1: note = Mod_Res - C-terminal is: 5cpaCO
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXXNXG

SEQ ID NO: 236 - protein, 14 aa, synthetic construct
  REGION 1..14: note = Modified peptide: 1071
  REGION 1..11: note = Disulfid - Disulfide bond
  REGION 3..7: note = Disulfid - Disulfide bond
  SITE 1: note = Mod_Res - C-terminal is: 5cpa
  SITE 1: note = misc_feature - Modified Amino Acid: dK
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: hE
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to E14
KXNTWKXXXXENXG

SEQ ID NO: 237 - protein, 13 aa, synthetic construct
  REGION 1..13: note = Modified peptide: 1181
  REGION 3..8: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 238 - protein, 13 aa, synthetic construct
  REGION 1..13: note = Modified peptide: 1183
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKSXG

SEQ ID NO: 239 - protein, 14 aa, synthetic construct
  REGION 1..14: note = Modified peptide: 1184
  REGION 1..14: note = Disulfid - Disulfide bond
  REGION 3..7: note = Disulfid - Disulfide bond
  SITE 1: note = Mod_Res - C-terminal is: ac
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: KAc
  SITE 13: note = misc_feature - Modified Amino Acid: 3Pya
  SITE 14: note = misc_feature - Modified Amino Acid: dK
  SITE 14: note = Mod_Res - N-terminal is: am
  REGION 2..7: note = Disulfid - Cyclic bond: Pen (X9) to Pen (X4)
  REGION 1..14: note = Disulfid - Cyclic bond: k to K14
EXNTWKXXXXKNXK

SEQ ID NO: 240 - protein, 13 aa, synthetic construct
  REGION 1..13: note = Modified peptide: 1198
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: hK(Me)3
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWKXXXXENXG

SEQ ID NO: 241 - protein, 13 aa, synthetic construct
  REGION 1..13: note = Modified peptide: 1199
  REGION 3..8: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: Pen
  SITE 4: note = misc_feature - Modified Amino Acid: 7MeW
  SITE 5: note = misc_feature - Modified Amino Acid: K(NMeAc)
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWKXXXXENXG

SEQ ID NO: 242 - protein, 13 aa, synthetic construct
  REGION 1..13: note = Modified peptide: 1200
  REGION 3..8: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: Pen
  SITE 4: note = misc_feature - Modified Amino Acid: 7MeW
  SITE 5: note = misc_feature - Modified Amino Acid: K(NMeAc)
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 10: note = misc_feature - Modified Amino Acid: K(NMeAc)
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWKXXXXKNXG

SEQ ID NO: 243 - protein, 13 aa, synthetic construct
  REGION 1..13: note = Modified peptide: 1226
  REGION 3..8: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: Pen
  SITE 4: note = misc_feature - Modified Amino Acid: 7MeW
  SITE 5: note = misc_feature - Modified Amino Acid: K(NMeAc)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXKNXG

SEQ ID NO: 244 - protein, 13 aa, synthetic construct
  REGION 1..13: note = Modified peptide: 1227
  REGION 3..8: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: Pen
  SITE 4: note = misc_feature - Modified Amino Acid: 7MeW
  SITE 5: note = misc_feature - Modified Amino Acid: K(Me)3
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 10: note = misc_feature - Modified Amino Acid: K(Me)3
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWKXXXXKNXG

SEQ ID NO: 245 - protein, 13 aa, synthetic construct
  REGION 1..13: note = Modified peptide: 1228
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: K(Me)3
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 10: note = misc_feature - Modified Amino Acid: K(Me)3
  SITE 12: note = misc_feature - Modified Amino Acid: 3Pya
  SITE 13: note = misc_feature - Modified Amino Acid: MeGly
  SITE 13: note = Mod_Res - N-terminal is: CON(Me)2
  REGION 1..6: note = Disulfid - Cyclic bond: Pen (X9) to Pen (X4)
  REGION 3..8: note = Disulfid - Cyclic bond at R1: E (X10) to AEF(NMe) (X5)
XETWKXXXXKNXG

SEQ ID NO: 246 - protein, 14 aa, synthetic construct
  REGION 1..14: note = Modified peptide: 1263
  REGION 1..11: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: dK
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: hE
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 1..11: note = Disulfid - Cyclic bond: K to E14
KXNTWKXXXXENXG

SEQ ID NO: 247 - protein, 14 aa, synthetic construct
  REGION 1..14: note = Modified peptide: 1283
  REGION 1..11: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: dK
  SITE 2: note = misc_feature - Modified Amino Acid: Pen
  SITE 5: note = misc_feature - Modified Amino Acid: 7MeW
  SITE 6: note = misc_feature - Modified Amino Acid: K(NMeAc)
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: hE
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 1..11: note = Disulfid - Cyclic bond: K to E14
KXNTWKXXXXENXG

SEQ ID NO: 248 - protein, 13 aa, synthetic construct
  REGION 1..13: note = Modified peptide: 1284
  REGION 1..11: note = Disulfid - Disulfide bond
  REGION 3..7: note = Disulfid - Disulfide bond
  SITE 1: note = Mod_Res - C-terminal is: PEG2 (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: K(NMeAc)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: hE (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: PEG2 (X13) to hE (R1)
XNTWKXXXXENXG

SEQ ID NO: 249 - protein, 13 aa, synthetic construct
  REGION 1..13: note = Modified peptide: 1346
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: K(mPEG12)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: KAc
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWXXXXXKLXG

SEQ ID NO: 250 - protein, 14 aa, synthetic construct
  REGION 1..14: note = Modified peptide: 1384
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dK(Me)3
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF(NMe)
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: CON(Me)2
  REGION 2..7: note = Disulfid - Cyclic bond: Pen (X9) to Pen (X4)
  REGION 3..8: note = Disulfid - Cyclic bond at R1: E (X10) to AEF(NMe) (X5)14
KXETWKXXXXENXG

SEQ ID NO: 251 - protein, 13 aa, synthetic construct
  REGION 1..13: note = Modified peptide: 1400
  REGION 8..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 6MeQui
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
XNTWKXXXXXNXG

SEQ ID NO: 252 - protein, 13 aa, synthetic construct
  REGION 1..13: note = Modified peptide: 1643
  REGION 1..11: note = Disulfid - Disulfide bond
  REGION 3..7: note = Disulfid - Disulfide bond
  SITE 1: note = Mod_Res - C-terminal is: AEEP (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: AEEP (X13) to E (R1)
XNTWKXXXXXNXG

SEQ ID NO: 253 - protein, 13 aa, synthetic construct
  REGION 1..13: note = Modified peptide: 1789
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 5: note = misc_feature - Modified Amino Acid: K(PEG2PEG2gEC18OH)
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)
XNTWXCXXXXNXG

SEQ ID NO: 254 - protein, 14 aa, synthetic construct
  REGION 1..14: note = Modified peptide: 1794
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 14: note = misc_feature - Modified Amino Acid: K(PEG2PEG2gEC18OH)
  SITE 14: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)14
XNTWQCXXXXNXGX

SEQ ID NO: 255 - protein, 13 aa, synthetic construct
  REGION 1..13: note = Modified peptide: 1795
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)
XNTWQCXXXXNHG

SEQ ID NO: 256 - protein, 13 aa, synthetic construct
  REGION 1..13: note = Modified peptide: 1796
  REGION 1..11: note = Disulfid - Disulfide bond
  REGION 3..7: note = Disulfid - Disulfide bond
  SITE 1: note = Mod_Res - C-terminal is: 5Ava (X13)
  SITE 1: note = misc_feature - Modified Amino Acid: Abu
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3MeH
  SITE 13: note = misc_feature - Modified Amino Acid: MeGly
  SITE 13: note = Mod_Res - N-terminal is: am
  REGION 1..6: note = Disulfid - Cyclic bond: Abu (X9) to C
  REGION 1..11: note = Disulfid - Cyclic bond at R1: 5Ava (X13) to E (R1)
XNTWQCXXXXNHG

SEQ ID NO: 257 - protein, 14 aa, synthetic construct
  REGION 1..14: note = Modified peptide: 1832
  REGION 1..11: note = Disulfid - Disulfide bond
  REGION 3..7: note = Disulfid - Disulfide bond
  SITE 1: note = Mod_Res - C-terminal is: AEEP (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 14: note = misc_feature - Modified Amino Acid: K(PEG2PEG2gEC18OH)
  SITE 14: note = Mod_Res - N-terminal is: am
  REGION 1..6: note = Disulfid - Cyclic bond: Pen (X9) to Pen (X4)
  REGION 1..11: note = Disulfid - Cyclic bond at R1: AEEP (X13) to E (R1)14
XNTWKXXXXXNXGX

SEQ ID NO: 258 - protein, 14 aa, synthetic construct
  REGION 1..14: note = Modified peptide: 1839
  REGION 3..8: note = Disulfid - Disulfide bond
  REGION 3..7: note = Disulfid - Disulfide bond
  SITE 1: note = Mod_Res - C-terminal is: HOC18gEPEG2PEG2CO
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 3..8: note = Disulfid - Cyclic bond at R1: E (X10) to AEF (X5)14
RXETWKXXXXENXG

SEQ ID NO: 259 - protein, 14 aa, synthetic construct
  REGION 1..14: note = Modified peptide: 1840
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dK(PEG2PEG2gEC18OH)
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 3..8: note = Disulfid - Cyclic bond at R1: E (X10) to AEF (X5)14
XXETWKXXXXENXG

SEQ ID NO: 260 - protein, 15 aa, synthetic construct
  REGION 1..15: note = Modified peptide: 1844
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 15: note = misc_feature - Modified Amino Acid: K(PEG2PEG2gEC18OH)
  SITE 15: note = Mod_Res - N-terminal is: am
  REGION 2..7: note = Disulfid - Cyclic bond: Pen (X9) to Pen (X4)
  REGION 3..8: note = Disulfid - Cyclic bond at R1: E (X10) to AEF (X5)15
RXETWKXXXXENXGX

SEQ ID NO: 261 - protein, 13 aa, synthetic construct
  REGION 1..13: note = Modified peptide: 1846
  REGION 1..11: note = Disulfid - Disulfide bond
  REGION 3..7: note = Disulfid - Disulfide bond
  SITE 1: note = Mod_Res - C-terminal is: AEEP (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: K(PEG2PEG2gEC18OH)
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: AEEP (X13) to E (R1)
XNTWXXXXXXNXG

SEQ ID NO: 262 - protein, 13 aa, synthetic construct
  REGION 1..13: note = Modified peptide: 1847
  REGION 1..11: note = Disulfid - Disulfide bond
  REGION 3..7: note = Disulfid - Disulfide bond
  SITE 1: note = Mod_Res - C-terminal is: AEEP (X13)
  SITE 1: note = misc_feature - Modified Amino Acid: Pen
  SITE 2: note = misc_feature - Modified Amino Acid: K(PEG2PEG2gEC18OH)
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: E (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: AEEP (X13) to E (R1)
XXTWKXXXXXNXG

SEQ ID NO: 263 - protein, 14 aa, synthetic construct
  REGION 1..14: note = Modified peptide: 1853
  REGION 1..11: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: dhE
  SITE 2: note = misc_feature - Modified Amino Acid: Pen
  SITE 3: note = misc_feature - Modified Amino Acid: N(N(Me)2)
  SITE 5: note = misc_feature - Modified Amino Acid: 7MeW
  SITE 6: note = misc_feature - Modified Amino Acid: K(NMeAc)
  SITE 7: note = misc_feature - Modified Amino Acid: Pen
  SITE 8: note = misc_feature - Modified Amino Acid: TMAPF
  SITE 9: note = misc_feature - Modified Amino Acid: 2Nal
  SITE 10: note = misc_feature - Modified Amino Acid: THP
  SITE 11: note = misc_feature - Modified Amino Acid: KNMe
  SITE 13: note = misc_feature - Modified Amino Acid: 3Pya
  SITE 14: note = misc_feature - Modified Amino Acid: MeGly
  SITE 14: note = Mod_Res - N-terminal is: CON(Me)2
  REGION 2..7: note = Disulfid - Cyclic bond: Pen (X9) to Pen (X4)
  REGION 1..11: note = Disulfid - Cyclic bond: dhE (X13) to K14
XXNTWKXXXXKNXG

SEQ ID NO: 264 - protein, 14 aa, synthetic construct
  REGION 1..14: note = Modified peptide: 1854
  REGION 1..11: note = Disulfid - Disulfide bond
  REGION 3..7: note = Disulfid - Disulfide bond
  SITE 1: note = Mod_Res - C-terminal is: cPEG3aCO
  SITE 1: note = misc_feature - Modified Amino Acid: dK
  SITE 2: note = misc_feature - Modified Amino Acid: Pen
  SITE 3: note = misc_feature - Modified Amino Acid: N(N(Me)2)
  SITE 5: note = misc_feature - Modified Amino Acid: 7MeW
  SITE 6: note = misc_feature - Modified Amino Acid: K(NMeAc)
  SITE 7: note = misc_feature - Modified Amino Acid: Pen
  SITE 8: note = misc_feature - Modified Amino Acid: TMAPF
  SITE 9: note = misc_feature - Modified Amino Acid: 2Nal
  SITE 10: note = misc_feature - Modified Amino Acid: THP
  SITE 11: note = misc_feature - Modified Amino Acid: hE
  SITE 13: note = misc_feature - Modified Amino Acid: 3Pya
  SITE 14: note = misc_feature - Modified Amino Acid: MeGly
  SITE 14: note = Mod_Res - N-terminal is: CON(Me)2
  REGION 2..7: note = Disulfid - Cyclic bond: Pen (X9) to Pen (X4)
  REGION 1..11: note = Disulfid - Cyclic bond: K to E14
KXNTWKXXXXENXG

SEQ ID NO: 265 - protein, 13 aa, synthetic construct
  REGION 1..13: note = Modified peptide: 1907
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 2: note = misc_feature - Modified Amino Acid: K(NMe)
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF(NMe)
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: K(NMe) (X10) to AEF(NMe) (X5)
XKTWKXXXXENXG

SEQ ID NO: 266 - protein, 13 aa, synthetic construct
  REGION 1..13: note = Modified peptide: 2015
  REGION 1..11: note = Disulfid - Disulfide bond
  REGION 3..7: note = Disulfid - Disulfide bond
  SITE 1: note = Mod_Res - C-terminal is: GABA (X13)
  SITE 1: note = misc_feature - Modified Amino Acid: dR
  SITE 2: note = misc_feature - Modified Amino Acid: Pen
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 11: note = misc_feature - Modified Amino Acid: E (R1)
  SITE 13: note = misc_feature - Modified Amino Acid: THP
  SITE 13: note = Mod_Res - N-terminal is: am
  REGION 2..7: note = Disulfid - Cyclic bond: Pen (X9) to Pen (X4)
  REGION 1..11: note = Disulfid - Cyclic bond at R1: GABA (X13) to E (R1)
RXQTWQXXXXENX

SEQ ID NO: 267 - protein, 14 aa, synthetic construct
  REGION 1..14: note = Modified peptide: 2122
  REGION 4..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: dR
  SITE 2: note = misc_feature - Modified Amino Acid: Pen
  SITE 5: note = misc_feature - Modified Amino Acid: 7MeW
  SITE 6: note = misc_feature - Modified Amino Acid: KAc
  SITE 7: note = misc_feature - Modified Amino Acid: Pen
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Pen (X9) to Pen (X4)
  REGION 3..8: note = Disulfid - Cyclic bond at R1: E (X10) to AEF (X5)14
RXETWKXXXXENXG

SEQ ID NO: 268 - protein, 13 aa, synthetic construct
  REGION 1..13: note = Modified peptide: 2123
  REGION 3..8: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 3..8: note = Disulfid - Cyclic bond at R1: E (X10) to AEF (X5)
XETWKXXXXENXG

SEQ ID NO: 269 - protein, 13 aa, synthetic construct
  REGION 1..13: note = Modified peptide: 2150
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Dbu
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: bAla
  SITE 13: note = Mod_Res - N-terminal is: am
  REGION 2..7: note = Disulfid - Cyclic bond: Abu (X9) to C
  REGION 1..11: note = Disulfid - Cyclic bond: Dbu (X13) to E
XXQTWQCXXXENA

SEQ ID NO: 270 - protein, 14 aa, synthetic construct
  REGION 1..14: note = Modified peptide: 2151
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: hK
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Abu (X9) to C
  REGION 1..11: note = Disulfid - Cyclic bond: K to E14
KXQTWQCXXXENXG

SEQ ID NO: 271 - protein, 14 aa, synthetic construct
  REGION 1..14: note = Modified peptide: 2152
  REGION 1..11: note = Disulfid - Disulfide bond
  REGION 3..7: note = Disulfid - Disulfide bond
  SITE 1: note = Mod_Res - C-terminal is: ac
  SITE 1: note = misc_feature - Modified Amino Acid: Dbu
  SITE 2: note = misc_feature - Modified Amino Acid: Abu
  SITE 8: note = misc_feature - Modified Amino Acid: AEF
  SITE 9: note = misc_feature - Modified Amino Acid: 2Nal
  SITE 10: note = misc_feature - Modified Amino Acid: THP
  SITE 13: note = misc_feature - Modified Amino Acid: 3Pya
  SITE 14: note = misc_feature - Modified Amino Acid: MeGly
  SITE 14: note = Mod_Res - N-terminal is: am
  REGION 2..7: note = Disulfid - Cyclic bond: Abu (X9) to C
  REGION 1..11: note = Disulfid - Cyclic bond: Dbu (X13) to E14
XXQTWQCXXXENXG

SEQ ID NO: 272 - protein, 13 aa, synthetic construct
  REGION 1..13: note = Modified peptide: 2164
  REGION 1..11: note = Disulfid - Disulfide bond
  REGION 3..7: note = Disulfid - Disulfide bond
  SITE 1: note = Mod_Res - C-terminal is: AEEP (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: hSer (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: AEEP (X13) to hSer (R1)
XNTWKXXXXSNXG

SEQ ID NO: 273 - protein, 13 aa, synthetic construct
  REGION 1..13: note = Modified peptide: 2165
  REGION 1..11: note = Disulfid - Disulfide bond
  REGION 3..7: note = Disulfid - Disulfide bond
  SITE 1: note = Mod_Res - C-terminal is: AEEP(Ns) (X13)
  SITE 1: note = misc_feature - Modified Amino Acid: Pen
  SITE 4: note = misc_feature - Modified Amino Acid: 7MeW
  SITE 5: note = misc_feature - Modified Amino Acid: KAc
  SITE 6: note = misc_feature - Modified Amino Acid: Pen
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: hSer (R1)
  SITE 12: note = misc_feature - Modified Amino Acid: 3Pya
  SITE 13: note = misc_feature - Modified Amino Acid: MeGly
  SITE 13: note = Mod_Res - N-terminal is: am
  REGION 1..6: note = Disulfid - Cyclic bond: Pen (X9) to Pen (X4)
  REGION 1..11: note = Disulfid - Cyclic bond at R1: AEEP(Ns) (X13) to hSer (R1)
XNTWKXXXXSNXG

SEQ ID NO: 274 - protein, 13 aa, synthetic construct
  REGION 1..13: note = Modified peptide: Formula
  SITE 1: note = misc_feature - Modified Amino Acid: Any AA
  SITE 2: note = misc_feature - Modified Amino Acid: Any AA
  SITE 4: note = misc_feature - Modified Amino Acid: Any AA
  SITE 5: note = misc_feature - Modified Amino Acid: Any AA
  SITE 6: note = misc_feature - Modified Amino Acid: Any AA
  SITE 7: note = misc_feature - Modified Amino Acid: AEF
  SITE 8: note = misc_feature - Modified Amino Acid: Any AA
  SITE 9: note = misc_feature - Modified Amino Acid: Any AA
  SITE 10: note = misc_feature - Modified Amino Acid: Any AA
  SITE 12: note = misc_feature - Modified Amino Acid: Any AA
  SITE 13: note = misc_feature - Modified Amino Acid: meG
XXTXXXFXXXNXG

SEQ ID NO: 275 - protein, 14 aa, synthetic construct
  REGION 1..14: note = Modified peptide: Formula
  SITE 1: note = misc_feature - Modified Amino Acid: Any AA
  SITE 2: note = misc_feature - Modified Amino Acid: Any AA
  SITE 3: note = misc_feature - Modified Amino Acid: Any AA
  SITE 5: note = misc_feature - Modified Amino Acid: Any AA
  SITE 6: note = misc_feature - Modified Amino Acid: K(Ac)
  SITE 7: note = misc_feature - Modified Amino Acid: Any AA
  SITE 8: note = misc_feature - Modified Amino Acid: AEF
  SITE 9: note = misc_feature - Modified Amino Acid: Any AA
  SITE 10: note = misc_feature - Modified Amino Acid: THP
  SITE 11: note = misc_feature - Modified Amino Acid: Any AA
  SITE 13: note = misc_feature - Modified Amino Acid: 3Pya
  SITE 14: note = misc_feature - Modified Amino Acid: Any AA
XXXTXKXFXXXNAX

SEQ ID NO: 276-277 - skipped (2 entries)

SEQ ID NO: 278 - protein, 13 aa, synthetic construct
  REGION 1..13: note = Modified peptide: Formula
  SITE 1: note = misc_feature - Modified Amino Acid: Any AA
  SITE 4: note = misc_feature - Modified Amino Acid: Any AA
  SITE 5: note = misc_feature - Modified Amino Acid: Any AA
  SITE 6: note = misc_feature - Modified Amino Acid: Any AA
  SITE 7: note = misc_feature - Modified Amino Acid: F4CONH2
  SITE 8: note = misc_feature - Modified Amino Acid: Any AA
  SITE 9: note = misc_feature - Modified Amino Acid: THP
  SITE 10: note = misc_feature - Modified Amino Acid: Any AA
  SITE 12: note = misc_feature - Modified Amino Acid: 3Pya
  SITE 13: note = misc_feature - Modified Amino Acid: meG
XNTXXXXXXXNAG

SEQ ID NO: 279 - protein, 13 aa, synthetic construct
  REGION 1..13: note = Modified peptide: Formula
  SITE 1: note = misc_feature - Modified Amino Acid: Any AA
  SITE 3: note = misc_feature - Modified Amino Acid: Any AA
  SITE 5: note = misc_feature - Modified Amino Acid: Any AA
  SITE 6: note = misc_feature - Modified Amino Acid: Any AA
  SITE 8: note = misc_feature - Modified Amino Acid: AEF
  SITE 9: note = misc_feature - Modified Amino Acid: Any AA
  SITE 10: note = misc_feature - Modified Amino Acid: THP
  SITE 11: note = misc_feature - Modified Amino Acid: Any AA
  SITE 13: note = misc_feature - Modified Amino Acid: Any AA
XAXTXXAFXXXNX

SEQ ID NO: 280 - protein, 14 aa, synthetic construct
  REGION 1..14: note = Modified peptide: Formula
  SITE 1: note = misc_feature - Modified Amino Acid: Any AA
  SITE 2: note = misc_feature - Modified Amino Acid: Any AA
  SITE 5: note = misc_feature - Modified Amino Acid: Any AA
  SITE 6: note = misc_feature - Modified Amino Acid: K(Ac)
  SITE 7: note = misc_feature - Modified Amino Acid: Any AA
  SITE 8: note = misc_feature - Modified Amino Acid: Any AA
  SITE 9: note = misc_feature - Modified Amino Acid: Any AA
  SITE 10: note = misc_feature - Modified Amino Acid: THP
  SITE 11: note = misc_feature - Modified Amino Acid: Any AA
  SITE 13: note = misc_feature - Modified Amino Acid: 3Pya
  SITE 14: note = misc_feature - Modified Amino Acid: Any AA
XXNTXKXXXXXNAX

SEQ ID NO: 281 - protein, 10 aa, synthetic construct
  REGION 1..10: note = Modified peptide: Formula
  SITE 1: note = misc_feature - Modified Amino Acid: Any AA
  SITE 3: note = misc_feature - Modified Amino Acid: Any AA
  SITE 4: note = misc_feature - Modified Amino Acid: Any AA
  SITE 6: note = misc_feature - Modified Amino Acid: AEF
  SITE 7: note = misc_feature - Modified Amino Acid: Any AA
  SITE 8: note = misc_feature - Modified Amino Acid: THP
  SITE 9: note = misc_feature - Modified Amino Acid: Any AA
  SITE 10: note = misc_feature - Modified Amino Acid: 3Pya
XTXXAFXXXA

SEQ ID NO: 282 - protein, 13 aa, synthetic construct
  REGION 1..13: note = Modified peptide: Formula
  SITE 1: note = misc_feature - Modified Amino Acid: Any AA
  SITE 4: note = misc_feature - Modified Amino Acid: Any AA
  SITE 5: note = misc_feature - Modified Amino Acid: Any AA
  SITE 6: note = misc_feature - Modified Amino Acid: Any AA
  SITE 7: note = misc_feature - Modified Amino Acid: AEF
  SITE 8: note = misc_feature - Modified Amino Acid: 2Nal
  SITE 9: note = misc_feature - Modified Amino Acid: Any AA
  SITE 10: note = misc_feature - Modified Amino Acid: Any AA
  SITE 12: note = misc_feature - Modified Amino Acid: 3Pya
  SITE 13: note = misc_feature - Modified Amino Acid: Any AA
XNTXXXFAXXNAX

SEQ ID NO: 283 - skipped

SEQ ID NO: 284 - protein, 14 aa, synthetic construct
  REGION 1..14: note = Modified peptide: Formula
  SITE 1: note = misc_feature - Modified Amino Acid: Any AA
  SITE 2: note = misc_feature - Modified Amino Acid: Any AA
  SITE 4: note = misc_feature - Modified Amino Acid: Any AA
  SITE 5: note = misc_feature - Modified Amino Acid: Any AA
  SITE 6: note = misc_feature - Modified Amino Acid: Any AA
  SITE 7: note = misc_feature - Modified Amino Acid: AEF
  SITE 8: note = misc_feature - Modified Amino Acid: 2Nal
  SITE 9: note = misc_feature - Modified Amino Acid: THP
  SITE 10: note = misc_feature - Modified Amino Acid: Any AA
  SITE 12: note = misc_feature - Modified Amino Acid: Any AA
  SITE 13: note = misc_feature - Modified Amino Acid: Any AA
  SITE 14: note = misc_feature - Modified Amino Acid: Any AA
XXTXXXFAXXNXXX

SEQ ID NO: 285 - skipped

SEQ ID NO: 286 - protein, 15 aa, synthetic construct
  REGION 1..15: note = Modified peptide: Formula
  SITE 1: note = misc_feature - Modified Amino Acid: Any AA
  SITE 2: note = misc_feature - Modified Amino Acid: Any AA
  SITE 3: note = misc_feature - Modified Amino Acid: Any AA
  SITE 5: note = misc_feature - Modified Amino Acid: Any AA
  SITE 6: note = misc_feature - Modified Amino Acid: Any AA
  SITE 7: note = misc_feature - Modified Amino Acid: Any AA
  SITE 8: note = misc_feature - Modified Amino Acid: AEF
  SITE 9: note = misc_feature - Modified Amino Acid: Any AA
  SITE 10: note = misc_feature - Modified Amino Acid: THP
  SITE 11: note = misc_feature - Modified Amino Acid: Any AA
  SITE 13: note = misc_feature - Modified Amino Acid: 3Pya
  SITE 14: note = misc_feature - Modified Amino Acid: meG
  SITE 15: note = misc_feature - Modified Amino Acid: Any AA
XXXTXXXFXXXNAGX